(19) United States
(12) Patent Application Publication
(10) Pub. No.: US 2021/0102184 A1
(43) Pub. Date: Apr. 8, 2021

(54) DETERGENT COMPOSITION COMPRISING XANTHAN LYASE AND ENDOGLUCANASE VARIANTS

(71) Applicant: Henkel AG & Co. KGaA, Duesseldorf (DE)

(72) Inventors: Nina MUSSMANN, Willich (DE); Susanne WIELAND, Dormagen/Zons (DE); Christian DEGERING, Erkrath (DE)

(21) Appl. No.: 16/970,005

(22) PCT Filed: Jan. 16, 2019
(86) PCT No.: PCT/EP2019/051016

§ 371 Date: Aug. 14, 2020

(30) Foreign Application Priority Data
Feb. 23, 2018 (DE) 10 2018 104 165.9

(51) Int. Cl. C12N 9/42; C12N 9/88; C11D 3/386; C11D 11/00

(52) U.S. Cl. CPC C12N 9/2437; C12Y 302/01004; C12N 9/88; C11D 11/0017; C11D 3/38636; C11D 11/0023; C12Y 402/02012

(57) ABSTRACT

The present invention relates to a detergent composition comprising endoglucanase variants and xanthan lyase variants and methods for use of said compositions.

### REFERENCE TO A SEQUENCE LISTING This application contains a Sequence Listing in computer readable form, which is incorporated herein by reference.

### BACKGROUND OF THE INVENTION

### Field of the Invention

The present invention relates to a detergent composition, such as laundry compositions and dish wash compositions, including hand wash and automatic dish wash compositions, comprising xanthan lyase and endoglucanase variants exhibiting alterations relative to the respective parent xanthan lyase and endoglucanase, respectively, in one or more properties including: detergent stability (e.g. improved stability in a detergent composition, e.g. in the presence of a chelator, e.g. EDTA or citrate) and/or storage stability (e.g. improved storage stability in a detergent composition, e.g. in the presence of a chelator, e.g. EDTA or citrate). The present invention further relates to detergent compositions comprising xanthan lyase and endoglucanase variants having activity on xanthan gum. The invention also relates to methods for producing and using the compositions of the invention. The variants described herein are particularly suitable for use in cleaning processes and detergent compositions.

### Description of the Related Art

Xanthan gum is a polysaccharide derived from the bacterial coat of Xanthomonas campestris. It is produced by the fermentation of glucose, sucrose, or lactose by the Xanthomonas campestris bacterium. After a fermentation period, the polysaccharide is precipitated from a growth medium with isopropyl alcohol, dried, and ground into a fine powder. Later, it is added to a liquid medium to form the gum. Xanthan gum is a natural polysaccharide consisting of different sugars which are connected by several different bonds, such as □-D-mannosyl-□-D-1,4-glucuronosyl bonds and □-D-glucosyl-□-D-1,4-glucosyl bonds. Xanthan gum is at least partly soluble in water and forms highly viscous solutions or gels. Complete enzymatic degradation of xanthan gum requires several enzymatic activities including xanthan lyase activity and endo-□-1,4-glucanase activity. Xanthan lyases are enzymes that cleave the □-D-mannosyl-□-D-1,4-glucuronosyl bond of xanthan and have been described in the literature. Xanthan lyases are known in the art, e.g. two xanthan lyases have been isolated from Paenibacillus alginolyticus XL-1 (e.g. Ruijssenaars et al. (1999) ‘A pyruvated mannose-specific xanthan lyase involved in xanthan degradation by Paenibacillus alginolyticus XL-1’, Appl. Environ. Microbiol. 65(6): 2446-2452, and Ruijssenaars et al. (2000), ‘A novel gene encoding xanthan lyase of Paenibacillus alginolyticus strain XL-1’, Appl. Environ. Microbiol. 66(9): 3945-3950). Glycoside hydrolases are enzymes that catalyse the hydrolysis of the glycosyl bond to release smaller sugars. There are over 100 classes of glycoside hydrolases which have been classified, see Henrissat et al. (1991) ‘A classification of glycosyl hydrolases based on amino-acid sequence similarities’, J. Biochem. 280: 309-316 and the Uniprot website at www.cazy.org. The glycoside hydrolase family 9 (GH9) consists of over 70 different enzymes that are mostly endo-glucanases (EC 3.2.1.4), cellobiohydrolases (EC 3.2.1.91), β-glucosidases (EC 3.2.1.21) and exo-6-glucosaminidase (EC 3.2.1.165). In recent years, xanthan gum has been used as an ingredient in many consumer products including foods (e.g. as thickening agent in salad dressings and dairy products) and cosmetics (e.g. as stabilizer and thickener in toothpaste and make-up, creams and lotions to prevent ingredients from separating and to provide the right texture of the product). Further xanthan gum has found use in the oil industry as an additive to regulate the viscosity of drilling fluids etc. The widespread use of xanthan gum has led to a desire to degrade solutions, gels or mixtures containing xanthan gum thereby allowing easier removal of the byproducts. Endoglucanases and xanthan lyases for the degradation of xanthan gum and the use of such enzymes for cleaning purposes, such as the removal of xanthan gum containing stains, and in the drilling and oil industries are known in the art, e.g. WO2013/167581A1.

The known xanthan endoglucanase having SEQ ID NO:2 and the known xanthan lyase having SEQ ID NO:6 were both found to be sensitive to the presence of detergents with chelators. To improve the applicability and/or cost and/or the performance of such enzymes, there is an ongoing search for variants with altered properties, such as increased stability, e.g. improved stability in a detergent composition, e.g. in the presence of a chelator, e.g. EDTA or citrate, etc. However, mutagenesis of large enzymes followed by purification and functional analysis of mutant libraries can be very expensive and laborious.

### SUMMARY OF THE INVENTION

In some aspects, the present invention relates to a detergent composition comprising

(A) an endoglucanase variant, comprising an alteration (e.g. a substitution, deletion or insertion, preferably a substitution) at one or more positions in a (chelator-induced instability) region selected from the group consisting of: region 1 corresponding to amino acids 95 to 105 of SEQ ID NO:2, region 2 corresponding to amino acids 115 to 138 of SEQ ID NO:2, region 3 corresponding to amino acids 210 to 251 of SEQ ID NO:2, region 4 corresponding to amino acids 267 to 301 of SEQ ID NO:2, region 5 corresponding to amino acids 339 to 361 of SEQ ID NO:2, region 6 corresponding to amino acids 547 to 595 of SEQ ID NO:2, region 7 corresponding to amino acids 612 to 660 of SEQ ID NO:2, region 8 corresponding to amino acids 806 to 828 of SEQ ID NO:2, region 9 corresponding to amino acids 839 to 1042 of SEQ ID NO:2, and/or region 10 corresponding to amino acids 1 to 94 of SEQ ID NO:2, region 11 corresponding to amino acids 106 to 114 of SEQ ID NO:2, region 12 corresponding to amino acids 139 to 209 of SEQ ID NO:2, region 13 corresponding to amino acids 252 to 266 of SEQ ID NO:2, region 14 corresponding to amino acids 302 to 338 of SEQ ID NO:2, region 15 corresponding to amino acids 362 to 546 of SEQ ID NO:2, region 16 corresponding to amino acids 596 to 611 of SEQ ID NO:2, region 17 corresponding to amino acids 661 to 805 of SEQ ID NO:2, region 18 corresponding to amino acids 829 to 838 of SEQ ID NO:2, and region 19 corresponding to amino acids 1043 to 1055 of SEQ ID NO:2; and

(B) a xanthan lyase variant, comprising an alteration (e.g. a substitution, deletion or insertion, preferably a substitution) at one or more positions in a (chelator-induced instability) region selected from the group consisting of: region 1 corresponding to amino acids 154 to 176 of SEQ ID NO:6, region 2 corresponding to amino acids 614 to 658 of SEQ ID NO:6, region 3 corresponding to amino acids 731 to 803 of SEQ ID NO:6, region 4 corresponding to amino acids 807 to 846 of SEQ ID NO:6, region 5 corresponding to amino acids 872 to 885 of SEQ ID NO:6, region 6 corresponding to amino acids 903 to 1004 of SEQ ID NO:6, and/or region 7 corresponding to amino acids 1 to 153 of SEQ ID NO:6, region 8 corresponding to amino acids 177 to 613 of SEQ ID NO:6, region 9 corresponding to amino acids 659 to 730 of SEQ ID NO:6, region 10 corresponding to amino acids 804 to 806 of SEQ ID NO:6, region 11 corresponding to amino acids 847 to 871 of SEQ ID NO:6, region 12 corresponding to amino acids 886 to 902 of SEQ ID NO:6, and region 13 corresponding to amino acids 1005 to 1037 of SEQ ID NO:6.

In various aspects, the endoglucanase variant (A) has at least 60% and less than 100% sequence identity to SEQ ID NO:2; preferably said endoglucanase variant has activity on xanthan gum pre-treated with xanthan lyase; and/or the xanthan lyase variant (B) has at least 60% and less than 100% sequence identity to SEQ ID NO:6 preferably said xanthan lyase variant having an activity on xanthan gum.

In some aspects, the present invention defines a chelator-induced instability region of a parent endoglucanase (e.g. SEQ ID NO:2) or a parent xanthan lyase (e.g. SEQ ID NO:6) having one or more of the following features: in the presence of a chelator it is relatively less conformationally stable than one or more or all of its adjacent regions; and/or in the presence of a chelator it is relatively more exposed to said chelator than one or more or all of its adjacent regions; and/or in the presence of a chelator it is relatively more accessible to said chelator than one or more or all of its adjacent regions; and/or in the presence of a chelator it is relatively more conformationally dynamic than one or more or all of its adjacent regions; and/or in the presence of a chelator it is relatively more receptive to deuterium incorporation than one or more or all of its adjacent regions. In the present invention, when referring to “relatively” it means that the features of a given region as indicated above are based on observed differences between its features and/or properties in the presence of a chelator and its features and/or properties in the absence of a chelator (i.e. in order to determine the impact of a chelator, each region is compared to itself in the absence of said chelator and any changes are determined relative to its native features/properties in the absence of a chelator).

In some aspects, the present invention relates to a detergent composition, as defined herein, comprising an endoglucanase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:2.

In some aspects, the present invention relates to a detergent composition, as defined herein, comprising a xanthan lyase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:6.

In some aspects, the detergent composition comprises an endoglucanase variant, comprising an alteration (e.g. a substitution, deletion or insertion) at one or more positions in a region selected from the group consisting of:

i) region 1 corresponding to amino acids 95 to 105 of SEQ ID NO:2, e.g. said alteration at one or more positions selected from the group consisting of positions: 95, 96, 97, 98, 99, 100, 101, 102, 103, 104, 105, wherein said positions correspond to amino acid positions of SEQ ID NO:2 (e.g. using the numbering of SEQ ID NO:2),

ii) region 2 corresponding to amino acids 115 to 138 of SEQ ID NO:2, e.g. said alteration at one or more positions selected from the group consisting of positions: 115, 116, 117, 118, 119, 120, 121, 122, 123, 124, 125, 126, 127, 128, 129, 130, 131, 132, 133, 134, 135, 136, 137, 138, wherein said positions correspond to amino acid positions of SEQ ID NO:2 (e.g. using the numbering of SEQ ID NO:2),

iii) region 3 corresponding to amino acids 210 to 251 of SEQ ID NO:2, e.g. said alteration at one or more positions selected from the group consisting of positions: 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, wherein said positions correspond to amino acid positions of SEQ ID NO:2 (e.g. using the numbering of SEQ ID NO:2),

iv) region 4 corresponding to amino acids 267 to 301 of SEQ ID NO:2, e.g. said alteration at one or more positions selected from the group consisting of positions: 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, wherein said positions correspond to amino acid positions of SEQ ID NO:2 (e.g. using the numbering of SEQ ID NO:2),

v) region 5 corresponding to amino acids 339 to 361 of SEQ ID NO:2, e.g. said alteration at one or more positions selected from the group consisting of positions: 339, 340, 341, 342, 343, 344, 345, 346, 347, 348, 349, 350, 351, 352, 353, 354, 355, 356, 357, 358, 359, 360, 361, wherein said positions correspond to amino acid positions of SEQ ID NO:2 (e.g. using the numbering of SEQ ID NO:2),

vi) region 6 corresponding to amino acids 547 to 595 of SEQ ID NO:2, e.g. said alteration at one or more positions selected from the group consisting of positions: 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, wherein said positions correspond to amino acid positions of SEQ ID NO:2 (e.g. using the numbering of SEQ ID NO:2),

vii) region 7 corresponding to amino acids 612 to 660 of SEQ ID NO:2, e.g. said alteration at one or more positions selected from the group consisting of positions: 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, wherein said positions correspond to amino acid positions of SEQ ID NO:2 (e.g. using the numbering of SEQ ID NO:2),

viii) region 8 corresponding to amino acids 806 to 828 of SEQ ID NO:2, e.g. said alteration at one or more positions selected from the group consisting of positions: 806, 807, 808, 809, 810, 811, 812, 813, 814, 815, 816, 817, 818, 819, 820, 821, 822, 823, 824, 825, 826, 827, 828, wherein said positions correspond to amino acid positions of SEQ ID NO:2 (e.g. using the numbering of SEQ ID NO:2),

ix) region 9 corresponding to amino acids 839 to 1042 of SEQ ID NO:2, e.g. said alteration at one or more positions selected from the group consisting of positions: 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, wherein said positions correspond to amino acid positions of SEQ ID NO:2 (e.g. using the numbering of SEQ ID NO:2),

x) region 10 corresponding to amino acids 1 to 94 of SEQ ID NO:2, e.g. said alteration at one or more positions selected from the group consisting of positions: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, wherein said positions correspond to amino acid positions of SEQ ID NO:2 (e.g. using the numbering of SEQ ID NO:2),

xi) region 11 corresponding to amino acids 106 to 114 of SEQ ID NO:2, e.g. said alteration at one or more positions selected from the group consisting of positions: 107, 108, 109, 110, 111, 112, 113, 114, wherein said positions correspond to amino acid positions of SEQ ID NO:2 (e.g. using the numbering of SEQ ID NO:2),

xii) region 12 corresponding to amino acids 139 to 209 of SEQ ID NO:2, e.g. said alteration at one or more positions selected from the group consisting of positions: 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, wherein said positions correspond to amino acid positions of SEQ ID NO:2 (e.g. using the numbering of SEQ ID NO:2),

xiii) region 13 corresponding to amino acids 252 to 266 of SEQ ID NO:2, e.g. said alteration at one or more positions selected from the group consisting of positions: 252, 253, 254, 255, 256, 257, 258, 259, 260, 261, 262, 263, 264, 265, 266, wherein said positions correspond to amino acid positions of SEQ ID NO:2 (e.g. using the numbering of SEQ ID NO:2),

xiv) region 14 corresponding to amino acids 302 to 338 of SEQ ID NO:2, e.g. said alteration at one or more positions selected from the group consisting of positions: 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, wherein said positions correspond to amino acid positions of SEQ ID NO:2 (e.g. using the numbering of SEQ ID NO:2),

xv) region 15 corresponding to amino acids 362 to 546 of SEQ ID NO:2, e.g. said alteration at one or more positions selected from the group consisting of positions: 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, wherein said positions correspond to amino acid positions of SEQ ID NO:2 (e.g. using the numbering of SEQ ID NO:2),

xvi) region 16 corresponding to amino acids 596 to 611 of SEQ ID NO:2, e.g. said alteration at one or more positions selected from the group consisting of positions: 596, 597, 598, 599, 600, 601, 602, 603, 604, 605, 606, 607, 608, 609, 610, 611, wherein said positions correspond to amino acid positions of SEQ ID NO:2 (e.g. using the numbering of SEQ ID NO:2),

xvii) region 17 corresponding to amino acids 661 to 805 of SEQ ID NO:2, e.g. said alteration at one or more positions selected from the group consisting of positions: 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, , 781, 782, 783, 784, 785, 786, 787, 788, 789, 790, 791, 792, 793, 794, 795, 796, 797, 798, 799, 800, 801, 802, 803, 804, 805, wherein said positions correspond to amino acid positions of SEQ ID NO:2 (e.g. using the numbering of SEQ ID NO:2),

xviii) region 18 corresponding to amino acids 829 to 838 to 1042 of SEQ ID NO:2, e.g. said alteration at one or more positions selected from the group consisting of positions: 829, 830, 831, 832, 833, 834, 835, 836, 837, 838, wherein said positions correspond to amino acid positions of SEQ ID NO:2 (e.g. using the numbering of SEQ ID NO:2), and xix) region 19 corresponding to amino acids 1043 to 1055 of SEQ ID NO:2, e.g. said alteration at one or more positions selected from the group consisting of positions 1043, 1044, 1045, 1046, 1047, 1048, 1049, 1050, 1051, 1052, 1053, 1054, 1055, wherein said positions correspond to amino acid positions of SEQ ID NO:2 (e.g. using the numbering of SEQ ID NO:2).

In some aspects, the afore-mentioned variants have at least 60% and less than 100% sequence identity to SEQ ID NO:2, preferably said endoglucanase variant having activity on xanthan gum pre-treated with xanthan lyase.

In some aspects, the detergent composition comprises a xanthan lyase variant comprising an alteration (e.g. a substitution, deletion or insertion, preferably a substitution) at one or more positions in a region selected from the group consisting of:

i) region 1 corresponding to amino acids 154 to 176 of SEQ ID NO:6, e.g. said alteration at one or more positions selected from the group consisting of positions: 154, 155, 156, 157, 158, 159, 160, 161, 162, 163, 164, 165, 166, 167, 168, 169, 170, 171, 172, 173, 174, 175, 176, wherein said positions correspond to amino acid positions of SEQ ID NO:6 (e.g. using the numbering of SEQ ID NO:6),

ii) region 2 corresponding to amino acids 614 to 658 of SEQ ID NO:6, e.g. said alteration at one or more positions selected from the group consisting of positions: 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, wherein said positions correspond to amino acid positions of SEQ ID NO:6 (e.g. using the numbering of SEQ ID NO:6),

iii) region 3 corresponding to amino acids 731 to 803 of SEQ ID NO:6, e.g. said alteration at one or more positions selected from the group consisting of positions: 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, , 781, 782, 783, 784, 785, 786, 787, 788, 789, 790, 791, 792, 793, 794, 795, 796, 797, 798, 799, 800, 801, 802, 803, wherein said positions correspond to amino acid positions of SEQ ID NO:6 (e.g. using the numbering of SEQ ID NO:6),

iv) region 4 corresponding to amino acids 807 to 846 of SEQ ID NO:6, e.g. said alteration at one or more positions selected from the group consisting of positions: 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, wherein said positions correspond to amino acid positions of SEQ ID NO:6 (e.g. using the numbering of SEQ ID NO:6),

v) region 5 corresponding to amino acids 872 to 885 of SEQ ID NO:6, e.g. said alteration at one or more positions selected from the group consisting of positions: 872, 873, 874, 875, 876, 877, 878, 879, 880, 881, 882, 883, 884, 885, wherein said positions correspond to amino acid positions of SEQ ID NO:6 (e.g. using the numbering of SEQ ID NO:6),

vi) region 6 corresponding to amino acids 903 to 1004 of SEQ ID NO:6, e.g. said alteration at one or more positions selected from the group consisting of positions: 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, wherein said positions correspond to amino acid positions of SEQ ID NO:6 (e.g. using the numbering of SEQ ID NO:6), 6p vii) region 7 corresponding to amino acids 1 to 153 of SEQ ID NO:6, e.g. said alteration at one or more positions selected from the group consisting of positions: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152 and 153, wherein said positions correspond to amino acid positions of SEQ ID NO:6,

viii) region 8 corresponding to amino acids 177 to 613 of SEQ ID NO:6, e.g. said alteration at one or more positions selected from the group consisting of positions: 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612 and 613, wherein said positions correspond to amino acid positions of SEQ ID NO:6,

ix) region 9 corresponding to amino acids 659 to 730 of SEQ ID NO:6, e.g. said alteration at one or more positions selected from the group consisting of positions: 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729 and 730, wherein said positions correspond to amino acid positions of SEQ ID NO:6,

x) region 10 corresponding to amino acids 804 to 806 of SEQ ID NO:6, e.g. said alteration at one or more positions selected from the group consisting of positions: 804, 805 and 806, wherein said positions correspond to amino acid positions of SEQ ID NO:6,

xi) region 11 corresponding to amino acids 847 to 871 of SEQ ID NO:6, e.g. said alteration at one or more positions selected from the group consisting of positions: 847, 848, 849, 850, 851, 852, 853, 854, 855, 856, 857, 858, 859, 860, 861, 862, 863, 864, 865, 866, 867, 868, 869, 870 and 871, wherein said positions correspond to amino acid positions of SEQ ID NO:6,

xii) region 12 corresponding to amino acids 886 to 902 of SEQ ID NO:6, e.g. said alteration at one or more positions selected from the group consisting of positions: 886, 887, 888, 889, 890, 891, 892, 893, 894, 895, 896, 897, 898, 899, 900, 901 and 902, wherein said positions correspond to amino acid positions of SEQ ID NO:6, and

xiii) region 13 corresponding to amino acids 1005 to 1037 of SEQ ID NO:6, e.g. said alteration at one or more positions selected from the group consisting of positions: 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036 and 1037, wherein said positions correspond to amino acid positions of SEQ ID NO:6.

In some aspects, the afore-mentioned variants have at least 60% and less than 100% sequence identity to SEQ ID NO:6, preferably said xanthan lyase variant having an activity on xanthan gum.

In some aspects, the detergent composition comprises an endoglucanase variant, comprising an alteration (e.g. a substitution, deletion or insertion, preferably a substitution) at one or more positions in:

a) one or more regions selected from the group consisting of:

i) region 1 corresponding to amino acids 95 to 105 of SEQ ID NO:2, e.g. said alteration at one or more positions selected from the group consisting of positions: 95, 96, 97, 98, 99, 100, 101, 102, 103, 104, 105, wherein said positions correspond to amino acid positions of SEQ ID NO:2 (e.g. using the numbering of SEQ ID NO:2),

ii) region 2 corresponding to amino acids 115 to 138 of SEQ ID NO:2, e.g. said alteration at one or more positions selected from the group consisting of positions: 115, 116, 117, 118, 119, 120, 121, 122, 123, 124, 125, 126, 127, 128, 129, 130, 131, 132, 133, 134, 135, 136, 137, 138, wherein said positions correspond to amino acid positions of SEQ ID NO:2 (e.g. using the numbering of SEQ ID NO:2),

iii) region 3 corresponding to amino acids 210 to 251 of SEQ ID NO:2, e.g. said alteration at one or more positions selected from the group consisting of positions: 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, wherein said positions correspond to amino acid positions of SEQ ID NO:2 (e.g. using the numbering of SEQ ID NO:2),

iv) region 4 corresponding to amino acids 267 to 301 of SEQ ID NO:2, e.g. said alteration at one or more positions selected from the group consisting of positions: 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, wherein said positions correspond to amino acid positions of SEQ ID NO:2 (e.g. using the numbering of SEQ ID NO:2),

v) region 5 corresponding to amino acids 339 to 361 of SEQ ID NO:2, e.g. said alteration at one or more positions selected from the group consisting of positions: 339, 340, 341, 342, 343, 344, 345, 346, 347, 348, 349, 350, 351, 352, 353, 354, 355, 356, 357, 358, 359, 360, 361, wherein said positions correspond to amino acid positions of SEQ ID NO:2 (e.g. using the numbering of SEQ ID NO:2),

vi) region 6 corresponding to amino acids 547 to 595 of SEQ ID NO:2, e.g. said alteration at one or more positions selected from the group consisting of positions: 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, wherein said positions correspond to amino acid positions of SEQ ID NO:2 (e.g. using the numbering of SEQ ID NO:2),

vii) region 7 corresponding to amino acids 612 to 660 of SEQ ID NO:2, e.g. said alteration at one or more positions selected from the group consisting of positions: 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, wherein said positions correspond to amino acid positions of SEQ ID NO:2 (e.g. using the numbering of SEQ ID NO:2),

viii) region 8 corresponding to amino acids 806 to 828 of SEQ ID NO:2, e.g. said alteration at one or more positions selected from the group consisting of positions: 806, 807, 808, 809, 810, 811, 812, 813, 814, 815, 816, 817, 818, 819, 820, 821, 822, 823, 824, 825, 826, 827, 828, wherein said positions correspond to amino acid positions of SEQ ID NO:2 (e.g. using the numbering of SEQ ID NO:2), and

ix) region 9 corresponding to amino acids 839 to 1042 of SEQ ID NO:2, e.g. said alteration at one or more positions selected from the group consisting of positions: 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, wherein said positions correspond to amino acid positions of SEQ ID NO:2 (e.g. using the numbering of SEQ ID NO:2); and/or

b) one or more adjacent regions, said adjacent region being selected from the group consisting of: (i′) region 10 corresponding to amino acids 1 to 94 of SEQ ID NO:2; (ii') region 11 corresponding to amino acids 106 to 114 of SEQ ID NO:2; (iii′) region 12 corresponding to amino acids 139 to 209 of SEQ ID NO:2; (iv′) region 13 corresponding to amino acids 252 to 266 of SEQ ID NO:2; (v′) region 14 corresponding to amino acids 302 to 338 of SEQ ID NO:2; (vi′) region 15 corresponding to amino acids 362 to 546 of SEQ ID NO:2; (vii') region 16 corresponding to amino acids 596 to 611 of SEQ ID NO:2; (viii′) region 17 corresponding to amino acids 661 to 805 of SEQ ID NO:2; (ix′) region 18 corresponding to amino acids 829 to 838 of SEQ ID NO:2; and (x′) region 19 corresponding to amino acids 1043 to 1055 of SEQ ID NO:2.

In some aspects, the present invention relates to a detergent composition comprising an endoglucanase variant, comprising an alteration (e.g. a substitution, deletion or insertion, preferably a substitution) at one or more positions in:

a) one or more regions selected from the group consisting of:

i) region 10 corresponding to amino acids 1 to 94 of SEQ ID NO:2, e.g. said alteration at one or more positions selected from the group consisting of positions: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, wherein said positions correspond to amino acid positions of SEQ ID NO:2 (e.g. using the numbering of SEQ ID NO:2),

ii) region 11 corresponding to amino acids 106 to 114 of SEQ ID NO:2, e.g. said alteration at one or more positions selected from the group consisting of positions: 107, 108, 109, 110, 111, 112, 113, 114, wherein said positions correspond to amino acid positions of SEQ ID NO:2 (e.g. using the numbering of SEQ ID NO:2),

iii) region 12 corresponding to amino acids 139 to 209 of SEQ ID NO:2, e.g. said alteration at one or more positions selected from the group consisting of positions: 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, wherein said positions correspond to amino acid positions of SEQ ID NO:2 (e.g. using the numbering of SEQ ID NO:2),

iv) region 13 corresponding to amino acids 252 to 266 of SEQ ID NO:2, e.g. said alteration at one or more positions selected from the group consisting of positions: 252, 253, 254, 255, 256, 257, 258, 259, 260, 261, 262, 263, 264, 265, 266, wherein said positions correspond to amino acid positions of SEQ ID NO:2 (e.g. using the numbering of SEQ ID NO:2),

v) region 14 corresponding to amino acids 302 to 338 of SEQ ID NO:2, e.g. said alteration at one or more positions selected from the group consisting of positions: 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, wherein said positions correspond to amino acid positions of SEQ ID NO:2 (e.g. using the numbering of SEQ ID NO:2),

vi) region 15 corresponding to amino acids 362 to 546 of SEQ ID NO:2, e.g. said alteration at one or more positions selected from the group consisting of positions: 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, wherein said positions correspond to amino acid positions of SEQ ID NO:2 (e.g. using the numbering of SEQ ID NO:2),

vii) region 16 corresponding to amino acids 596 to 611 of SEQ ID NO:2, e.g. said alteration at one or more positions selected from the group consisting of positions: 596, 597, 598, 599, 600, 601, 602, 603, 604, 605, 606, 607, 608, 609, 610, 611, wherein said positions correspond to amino acid positions of SEQ ID NO:2 (e.g. using the numbering of SEQ ID NO:2),

viii) region 17 corresponding to amino acids 661 to 805 of SEQ ID NO:2, e.g. said alteration at one or more positions selected from the group consisting of positions: 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, , 781, 782, 783, 784, 785, 786, 787, 788, 789, 790, 791, 792, 793, 794, 795, 796, 797, 798, 799, 800, 801, 802, 803, 804, 805, wherein said positions correspond to amino acid positions of SEQ ID NO:2 (e.g. using the numbering of SEQ ID NO:2),

ix) region 18 corresponding to amino acids 829 to 838 to 1042 of SEQ ID NO:2, e.g. said alteration at one or more positions selected from the group consisting of positions: 829, 830, 831, 832, 833, 834, 835, 836, 837, 838, wherein said positions correspond to amino acid positions of SEQ ID NO:2 (e.g. using the numbering of SEQ ID NO:2)

x) region 19 corresponding to amino acids 1043 to 1055 of SEQ ID NO:2, e.g. said alteration at one or more positions selected from the group consisting of positions 1043, 1044, 1045, 1046, 1047, 1048, 1049, 1050, 1051, 1052, 1053, 1054, 1055, wherein said positions correspond to amino acid positions of SEQ ID NO:2 (e.g. using the numbering of SEQ ID NO:2); and/or

b) one or more adjacent regions, said adjacent region being selected from the group consisting of: (i′) region 1 corresponding to amino acids 95 to 105 of SEQ ID NO:2; (ii′) region 2 corresponding to amino acids 115 to 138 of SEQ ID NO:2; (iii′) region 3 corresponding to amino acids 210 to 251 of SEQ ID NO:2; (iv′) region 4 corresponding to amino acids 267 to 301 of SEQ ID NO:2; (v′) region 5 corresponding to amino acids 339 to 361 of SEQ ID NO:2; (vi′) region 6 corresponding to amino acids 547 to 595 of SEQ ID NO:2; (vii') region 7 corresponding to amino acids 612 to 660 of SEQ ID NO:2; (viii′) region 8 corresponding to amino acids 806 to 828 of SEQ ID NO:2; and (ix′) region 9 corresponding to amino acids 839 to 1042 of SEQ ID NO:2.

In a particular aspect, the endoglucanase variant as described herein is one that does not comprise any amino acid alteration at a position outside of regions 10, 11, 12, 13, 14, 15, 16, 17, 18, and 19. In this aspect, the endoglucanase variant thus does not comprise any alteration (e.g. a substitution, deletion or insertion, preferably a substitution) in a region selected from the group consisting of: region 1 corresponding to amino acids 95 to 105 of SEQ ID NO:2, region 2 corresponding to amino acids 115 to 138 of SEQ ID NO:2, region 3 corresponding to amino acids 210 to 251 of SEQ ID NO:2, region 4 corresponding to amino acids 267 to 301 of SEQ ID NO:2, region 5 corresponding to amino acids 339 to 361 of SEQ ID NO:2, region 6 corresponding to amino acids 547 to 595 of SEQ ID NO:2, region 7 corresponding to amino aicds 612 to 660 of SEQ ID NO:2, region 8 corresponding to amino acids 806 to 828 of SEQ ID NO:2, and region 9 corresponding to amino acids 839 to 1045 of SEQ ID NO:2. Alternatively, the endoglucanase variant as described herein is one that does not comprise any amino acid alteration at a position outside of regions 1, 2, 3, 4, 5, 6, 7, 8, and 9. In this aspect, the endoglucanase variant thus does not comprise any alteration (e.g. a substitution, deletion or insertion, preferably a substitution) in a region selected from the group consisting of: region 10, region 11, region 12, region 13, region 14, region 15, region 16, region 17, region 18, and region 19. It is however preferred that the endoglucanase variant comprises at least one alteration in any of regions 1-9 and at least one alteration in any one regions 10-19.

In some aspects, the detergent composition comprises an endoglucanase variant having an alteration (e.g. a substitution, deletion or insertion, preferably a substitution) at one or more positions in: (i) regions 6 and 17; (ii) regions 6, 15 and 17; (iii) regions 10, 12 and 15; (iv) regions 6, 7, 16, and 17; (v) region 6, 9, 10, 12, 15, and 17; (vi) region 14 and 15; (vii) region 9; (viii) 6, 7, 9, 14, 15, 16, and 17; or (ix) 3, 6, 7, 9, 14, 15, 16, and 17; wherein said variant preferably has no alternation in the other regions besides those mentioned.

In some aspects, the detergent composition comprises an endoglucanase variant having an alteration (e.g. a substitution, deletion or insertion, preferably a substitution) at one or more positions selected from the group consisting of positions: 4, 17, 18, 20, 51, 53, 55, 56, 60, 63, 71, 79, 87, 92, 99, 120, 125, 126, 130, 137, 182, 186, 189, 192, 213, 216, 221, 226, 228, 230, 231, 232, 233, 235, 240, 243, 247, 249, 278, 279, 281, 283, 285, 289, 292, 294, 298, 302, 311, 313, 333, 346, 353, 358, 386, 387, 388, 390, 403, 408, 410, 416, 441, 448, 451, 471, 472, 476, 489, 507, 512, 515, 538, 555, 556, 557, 558, 559, 560, 561, 562, 563, 564, 567, 568, 570, 575, 578, 579, 580, 581, 583, 589, 590, 591, 592, 593, 595, 598, 599, 602, 603, 605, 607, 609, 616, 627, 630, 631, 635, 636, 638, 639, 640, 641, 642, 643, 644, 651, 676, 683, 688, 690, 694, 698, 699, 706, 711, 713, 1719, 720, 744, 749, 754, 756, 760, , 781, 786, 797, 810, 811, 812, 815, 823, 824, 825, 827, 828, 833, 834, 835, 837, 843, 848, 868, 869, 870, 871, 872, 873, 874, 880, 881, 883, 884, 885, 887, 888, 890, 892, 894, 898, 905, 906, 912, 920, 921, 924, 926, 927, 928, 932, 933, 934, 935, 937, 938, 939, 940, 941, 942, 943, 946, 948, 950, 952, 953, 954, 956, 957, 960, 966, 971, 972, 980, 989, 991, 994, 995, 998, 999, 1006, 1009, 1010, 1011, 1029, 1030, 1031, 1032, 1035, 1037, 1038, 1040, 1041, 1042, 1044, 1045, and 1048, wherein numbering is according to SEQ ID NO:2.

In one alternative embodiment, the endoglucanase variant comprises an alteration in one or more positions selected from the group of: 285, 333, 353, 558, 633, 635, 638, 639, 994, 281, 563, 575, 921, 558+559+560+561+562, 558, 559, 560, 561, 562 125, 126, 130, 213, 221, 228, 230, 231, 232, 235, 240, 243, 249, 278, 292, 297, 346, 556, 564, 565, 567, 568, 569, 570, 576, 578, 579, 580, 583, 589, 590, 591, 592, 593, 616, 627, 630, 636, 641, 642, 643, 644, 651, 810, 811, 812, 815, 823, 824, 825, 827, 843, 870, 871, 872, 873, 874, 881, 883, 884, 885, 887, 894, 920, 932, 933, 934, 935, 937, 938, 939, 940, 941, 942, 943, 950, 952, 953, 954, 960, 964, 966, 971, 974, 989, 991, 995, 998, 1006, 1010, 1011, 1029, 1030, 1031, 1032, 1035, 1037, 1038, 1040, 1041, 1044, 1045, 559+579, 564+579, 562+579, 559+579+99, 99, 559+579+281, 281+559+579, 559+579+616, 559+579+636, 559+579+651, 559+579+948, 948, 559+579+1009, 1009, 559+579+627, 579+921, 559+579+921, 99+579, 579+651, 579+948, 579+1009, 559+579+934, 934, 559+579+921+934, 559+579+627, 559+579+627+616, 559+579+627, 559+579+921+651, 559+579+921+627, 559+579+921+636, 559+579+921+616, 559+579+921+636, 559+579+921+627+636, 559+579+636+651, 559+579+616+651, 559+579+616+636, 559+579+616+921+934, 559+579+651+627, 559+579+651+636, 559+579+651+627+636, 559+579+651+616, 559+579+651+921+934, 636+934, 636+921, 636+627, 636+579, 638+934, 638+921, 638+627, 638+579,627+51, 51,627+451, 451, 627+559,627+579, 579+934, 651+638, 570+651, 570+921, 570+627, 570+559, 570+579, 570+638, 570+579,570+638,570+651,570+636,570+934,570+638,570+921,570+627,570+559,570+885, 885+934,885+627, 559+579+636, 559+579+638, 559+579+870, 559+579+560, 559+579+564, 559+579+570, 559+579+570, 559+579+570, 559+579+570, 559+579+570, 559+579+570, 559+579+570, 559+579+570, 559+570+579, 559+570+579, 559+570+579, 559+570+579, 559+570+579, 559+570+579, 559+570+579, 559+570+579, 559+560+579, 559+579+651, 559+579+651+934, 559+579+638, 559+579+921, 559+579+616+921, 559+579+636, 559+579, 559+579, 559+579+921, 559+579+616, 638+934,627+636,627+934,570+579, 416+559+579+636, 128+559+579+627, 128+559+579+636, and 579+636 of SEQ ID NO:2.

In some aspects, the detergent composition comprises an endoglucanase variant having an alteration (e.g. a substitution, deletion or insertion, preferably a substitution) at one or more positions selected from the group consisting of: N285G, W333L, T353D, N558NP, N558F, T633V, D635L, D635M, D635T, F638Y, T639D, G994N, and K281T, G563E, 1575M, 1575A, K921D, N558K+A559K+S560F+T561P+G562W, N558K, A559K, S560F, T561P, G562W and 1125V, A126R, K130R, K213R, A221R, K228E, K2281, G230F, G230L, G230A, G230H, G230N, G230W, G230T, F231Y, F231N, V232R, V232G, H235D, N240Q, G243K, G243R, A249N, A278S, K281F, K281V, K281Y, K281H, K281Q, K281N, K281W, N285L, N285M, N285S, N285P, N285T, N285Y, N285H, N285K, N285D, N285W, N285R, T292F, T292L, T2921, T292V, T292S, T292P, T292Y, T292Q, T292N, T292K, T292D, T292G, F297L, A346H, G556S, N558D, N558M, N558Q, N5581, N558Y, N558H, A559N, A559F, A559M, A559P, A559Y, A559H, A559Q, A559D, A559R, A559G, A5591, A559S, S560P, S560K, S560G, S560D, T561P, T561E, T561Q, T561S, T561D, A5641, A564Y, A564H, A564Q, A564K, A564E, E565M, V567F, K568R, L569F, L569Y, L569D, L569E, P570F, P570L, P5701, P570M, P570V, P570S, P570T, P570A, P570Y, P570H, P570Q, P570N, P570K, P570E, P570W, P570R, P570G, 1575D, 1575E, 1576F, 1576M, 1576P, D578R, Y579F, Y579W, V580L, D583M, Q589G, P590S, P590T, P590E, E591L, G592D, S593P, S593H, S593Q, S593N, S593K, S593D, S593E, S593R, S616D, K627L, K627M, K627V, K627S, K627T, K627Q, K627R, 1630F, 1630V, 1630Y, D635A, D635P, D635N, D635K, D635E, D635G, D635W, S636L, S636M, S636A, S636H, S636Q, S636N, S636K, S636R, F6381, F638V, F638T, F638L, F638H, T639V, T639S, T639L, T6391, T639M, T639A, T639E, T639W, T639G, Y641E, S642T, S642N, N643D, N643H, N643T, T644F, A651P, S810R, A811S, V812F, V8121, V812M, V812W, V812R, N815V, N815Y, N815E, N815W, N815R, S823Q, A824T, T825N, T825W, T825A, T825D, V8271, V827M, V827S, T843V, D870F, D870L, D8701, D870M, D870V, D870S, D870T, D870Y, D870H, D870Q, D870N, D870K, D870E, D870W, D870R, D870G, P871F, P871L, P8711, P871M, P871V, P871S, P871T, P871A, P871Y, P871H, P871Q, T872S, T872F, T872A, T872Y, T872H, T872Q, T872N, T872K, T872D, T872E, T872W, T872R, T872G, D873K, D873E, T874V, T874S, T874P, T874A, T874H, T874Q, T874N, T874K, V881Q, T883K, Y884H, A885F, A885Q, A885N, T887L, T8871, T887S, T887H, T887R, K894E, N920D, K921R, K921E, T932A, N933V, N933S, Y934G, Y934M, Y934S, Y934A, Y934Q, Y934N, Y934E, Y934W, Y934R, T935W, A937F, A937V, A937S, A937T, A937Q, A937D, A937E, V9381, K9391, K939V, D940E, N941S, N941H, N941D, A942P, A942E, D943Y, D943H, R950V, R950H, R950N, F952S, F952W, N953Y, G954L, Y960F, A964N, A964C, N966P, N966C, G971A, Q974K, Q974C, Q9891, Q991L, Q9911, Q991M, Q991V, Q991T, Q991K, Q991C, S9951, S995V, S995Q, S995R, S995C, G998V, G998A, 51006T, 51006A, S1006K, 51006R, Y1010W, L1011M, L1011S, L1011A, L1011Q, L1011N, L1011D, L1011E, R1029N, F1030M, K10311, K1031S, K1031T, K1031H, V1032G, K1035A, A1037E, A1037W, 51038L, S10381, L1040N, L1040E, G1041F, L1044F, L1044S, L1044N, L1044W, P1045Q, P1045W, and A559N+Y579F, A559N, Y579F, A564E+Y579F, A564E, Y579F, A559N+Y579W, A559N, Y579W, G562P+Y579W, G562P, Y579W, A564D+Y579W, A564D, Y579W, A559N+Y579W+K99R, A559N, Y579W, K99R, A559N+Y579W+K281R, A559N, Y579W, K281R, K281R+A559N+Y579W, K281R, A559N, Y579W, A559N+Y579W+S616D, A559N, Y579W, S616D, A559N+Y579W+S636N, A559N, Y579W, S636N, A559N+Y579W+A651P, A559N, Y579W, A651P, A559N+Y579W+K948E, A559N, Y579W, K948E, A559N+Y579W+K1009E, A559N, Y579W, K1009E, A559N+Y579W+K627R, A559N, Y579W, K627R, Y579W+K921R, Y579W, K921R, A559N+Y579W+K921R, A559N, Y579W, K921R, K99R+Y579W, K99R, Y579W, Y579W+A651P, Y579W, A651P, Y579W+K948E, Y579W, K948E, Y579W+K1009E, Y579W, K1009E, A559N+Y579W+Y934G, A559N, Y579W, Y934G, A559N+Y579W+K921R+Y934G, A559N, Y579W, K921R, Y934G, A559N+Y579W+K627M, A559N, Y579W, K627M, A559N+Y579W+K627R+S616D, A559N, Y579W, K627R, S616D, A559N+Y579F+K627R, A559N, Y579F, K627R, A559N+Y579W+K921R+A651P, A559N, Y579W, K921R, A651P, A559N+Y579W+K921R+K627R, A559N, Y579W, K921R, K627R, A559N+Y579W+K921R+S636K, A559N, Y579W, K921R, S636K, A559N+Y579W+K921R+S616D, A559N, Y579W, K921R, S616D, A559N+Y579W+K921R+S636N, A559N, Y579W, K921R, S636N, A559N+Y579W+K921R+K627R+S636N, A559N, Y579W, K921R, K627R, S636N, A559N+Y579W+S636N+A651P, A559N, Y579W, S636N, A651P, A559N+Y579W+S616D+A651P, A559N, Y579W, S616D, A651P, A559N+Y579W+S616D+S636K, A559N, Y579W, S616D, S636K, A559N+Y579W+S616D+K921R+Y934G, A559N, Y579W, S616D, K921R, Y934G, A559N+Y579W+A651P+K627M, A559N, Y579W, A651P, K627M, A559N+Y579W+A651P+S636K, A559N, Y579W, A651P, S636K, A559N+Y579W+A651P+K627R+S636N, A559N, Y579W, A651P, K627R, S636N, A559N+Y579W+A651P+S616D, A559N, Y579W, A651P, S616D, A559N+Y579W+A651P+K921R+Y934G, A559N, Y579W, A651P, K921R, Y934G, S636N+Y934G, S636N, Y934G, S636N+K921R, S636N, K921R, S636N+K627R, S636N, K627R, S636N+Y579W, S636N, Y579W, F6381+Y934G, F6381, Y934G, F638I+K921R, F6381, K921R, F6381+K627R, F6381, K627R, F6381+Y579W, F6381, Y579W, K627R+K51Q, K627R, K51Q, K627R+K451S, K627R, K451S, K627R+A559N, K627R, A559N, K627R+Y579W, K627R, Y579W, Y579W+Y934G, Y579W, Y934G, A651P+F638I, A651P, F6381, P570Q+A651P, P570Q, A651P, P570Q+K921R, P570Q, K921R, P570Q+K627R, P570Q, K627R, P570Q+A559N, P570Q, A559N, P570Q+Y579W, P570Q, Y579W, P570Q+F6381, P570Q, F6381, P570K+Y579W, P570K, Y579W, P570K+F6381, P570K, F6381, P570T+A651P, P570T, A651P, P570T+S636N, P570T, S636N, P570T+Y934G, P570T, Y934G, P570T+F6381, P570T, F6381, P570T+K921R, P570T, K921R, P570T+K627R, P570T, K627R, P570T+A559N, P570T, A559N, P570T+A885F, P570T, A885F, A885F+Y934G, A885F, Y934G, A885F+K627R, A885F, K627R, A559N+Y579W+S636L, A559N, Y579W, S636L, A559N+Y579W+F6381, A559N, Y579W, F6381, A559N+Y579W+D870M, D870M, A559N+Y579W+S560P, S560P, A559N+Y579W+A5641, A5641, A559N+Y579W+P570N, P570N, A559N+Y579W+P570K, P570K, A559N+Y579W+P570R, P570R, A559N+Y579W+P570A, P570A, A559N+Y579W+P570T, P570T, A559N+Y579W+P570S, P570S, A559N+Y579W+P570Q, P570Q, A559N+Y579W+P570H, P570H, and N558E, A559P, A559N, A559H, T561P, A564E, P570A, P570Q, P570R, P570S, P570K, P570T, P570N, Y579W, Y579F, T581M, S616D, K627R, K627M, K627Q, S636N, S636Q, S636R, S636K, S636M, S636H, F6381, F638L, N643D, A651P, A651S, A885F, A885Q, K921R, Y934R, Y934G, N966C, L1011A, K10311, and A559N+P570A+Y579W, A559N+P570H+Y579W, A559N+P570K+Y579W, A559N+P570N+Y579W, A559N+P570Q+Y579W, A559N+P570R+Y579W, A559N+P570T+Y579W, A559N+P570T+Y579W, A559N+S560P+Y579W, A559N+Y579W+A651 P, A559N+Y579W+A651P+Y934G, A559N+Y579W+F6381, A559N+Y579W+K921R, A559N+Y579W+S616D+K921R, A559N+Y579W+S636N, A559N+Y579F, A559N+Y579W, A559N+Y579W+K921R, A559N+Y579W+S616D, F6381+Y934G, K627R+S636N, K627R+Y934G, P570K+Y579W, Q416D+A559N+Y579W+S636N, Q416D, S128X+A559N+Y579W+K627R, S128X, S128X+A559N+Y579W+S636N, Y579W+S636N, V4T, S17A, N18G, F20P, F2ON, F20G, F20Y, K51Q, K51H, E53Y, E53P, E53G, Y55M, Y55D, V56M, Y60F, S63F, A71E, 579W, T87R, T92S, A120P, N129D, F137L, H182Y, A186P, N189K, K192N, N216D, N216Q, N216R, L226K, G230H, L233H, D247N, G279E, K281R, A283D, N285D, N285G, Q289E, T292A, T292F, T292Y, A294V, Q298E, 1302D, 1302H, 1302V, 1302M, H311N, S313D, A346D, A386P, 1387T, K388R, K390Q, 1403Y, E408D, E408N, E4085, E408P, E408A, E408G, P410G, Q4165, Q416D, N441G, A448E, A448W, A4485, K451S, K451Q, G471S, S472Y, D476R, Q489P, K507R, K512P, S515V, S538C, L555Q, G557R, N558E, A559N, A559P, A559H, A559D, S560P, 5560G, T561P, A564E, A5641, V567P, K568R, P570R, P570Q, P570K, P570A, P570T, P570G, P570S, P570H, P570N, 1575V, Y579W, Y579F, T581M, S593N, S593E, S595L, S598Q, A599S, 1602T, 1602D, V603P, 5605T, S607C, G609E, S616G, S616D, K627R, K627M, K627Q, K631R, K631A, D635A, D635E, D635M, D635N, D635L, D635W, S636N, S636K, S636L, S636Q, S636R, S636M, 5636H, F638N, F6381, F638L, F638V, F638H, F638M, T639G, T6391, T639M, T639Y, T639W, T639P, T639E, T640S, S642N, S642T, N643D, N643H, A651P, A651S, D676H, Q683E, A688G, Y690F, T694A, T697G, R698W, T699A, T706Q, T7115, T711V, T711Y, K713R, W719R, K720H, K744H, K744Q, A749T, K754R, V756Y, V756H, 5760G, T, 781M, N786K, T797S, S810Q, A824D, T825G, N828D, N833D, Q834E, S835A, S835D, V8371, N848D, A868E, A869V, D870V, T872G, T872H, T872W, T872Q, R880K, V881Q, V881T, T883R, T883V, T883C, T883K, Y884H, A885N, A885Q, A885F, T887K, T887S, L888M, V890R, T892P, T892V, R898Q, N905D, F906A, Q912V, N920P, K921R, A924D, V926F, V926P, K927R, S928D, T932A, N933S, N933V, Y934G, Y934R, Y934Q, A937E, V9381, K939V, N941S, A942P, G946R, K948R, Q956Y, Q956S, A957L, A957P, N966C, T972K, M9801, G994D, T999R, L1011A, K10311, A1037E, S1038G, G1041R, Y1042N, and F1048W.

In some aspects, the present invention relates to a detergent composition comprising an endoglucanase variant having an alteration (e.g. a substitution, deletion or insertion, preferably a substitution) at one or more positions selected from the group consisting of positions: 17, 20, 51, 53, 55, 56, 60, 63, 79, 87, 192, 302, 387, 388, 390, 403, 408, 410, 416, 448, 451, 471, 472, 507, 512, 515, 538, 598, 602, 605, 609, 676, 694, 698, 699, 711, 754, 760, , 781, 786, 797, 834, and 835 of SEQ ID NO:2.

In some aspects, the present invention relates to a detergent composition comprising an endoglucanase variant having an alteration (e.g. a substitution, deletion or insertion, preferably a substitution) at one or more positions selected from the group consisting of: S17A, F20P, F20N, F20G, F20Y, K51Q, K51H, E53P, E53G, Y55M, V56M, Y60F, S63F, T87R, K192N, I302H, I302V, I302M, I387T, K388R, K390Q, I403Y, E408D, E408S, E408P, E408A, E408G, E408N, P410G, Q416S, Q416D, A448E, A448W, A448S, K451S, G471S, S472Y, K507R, K512P, S515V, S538C, Y579W, S598Q, I1602T, I602D, S605T, G609E, D676H, T694A, R698W, T699A, T711V, T711Y, K754R, S760G, T, 781M, N786K, T797S, Q834E, and S835D of SEQ ID NO:2.

In some aspects, the present invention relates to a detergent composition comprising an endoglucanase variant having an alteration (e.g. a substitution, deletion or insertion, preferably a substitution) in the positions selected from the group consisting of positions: 559+579+697; 512+559+579+697; 18+71+186+408+579+602+651+688+756; 18+189+408+559+579+688+697+756+921+934; 313+488; 880+905+921+934; 302+313+408+579+602+651+697+880+921+934; or 216+313+408+476+579+602+638+651+697+719+880+887+921+934 of SEQ ID NO:2.

In some aspects, the present invention relates to a detergent composition comprising an endoglucanase variant having an alteration (e.g. a substitution, deletion or insertion, preferably a substitution) selected from the group consisting of the following alterations: A559N+Y579W+T697G; K512P+A559N+Y579W+T697G; N18G+A71E+A186P+E408D+Y579W+1602T+A651P+A688G+V756Y; N18G+N189K+E408D+A559N+Y579W+A688G+T697G+V756Y+K921R+Y934G; S313D+E408D; R880K+N905D+K921R+Y934G; I302D+S313D+E408D+Y579W+I602T+A651P+T697G+R880K+K921R+Y934G; and N216Q+S313D+E408D+D476R+Y579W+I602T+F638N+A651P+T697G+W719R+R880K+T887K+K9 21R+Y934G of SEQ ID NO:2.

In some aspects, the present invention relates to a detergent composition comprising an endoglucanase variant having activity on xanthan gum pre-treated with xanthan lyase; preferably said activity comprises endoglucanase EC 3.2.1.4 activity, further preferably said activity is endoglucanase EC 3.2.1.4 activity.

In some aspects, the present invention relates to a detergent composition comprising an endoglucanase variant having an improved stability in a detergent composition compared to a parent endoglucanase (e.g. with SEQ ID NO:2).

In some aspects, the present invention relates to a detergent composition comprising an endoglucanase variant having a half-life which is improved over the parent endoglucanase or a wild-type endoglucanase.

In one embodiment, the endoglucanase variant has a half-life of at least 1.5 h when measured at a temperature of 25° C. and in a detergent concentration of 90%. In a particular embodiment, the half-life is measured as described in Examples 3 and 7.

In some aspects, the present invention relates to a detergent composition comprising an endoglucanase variant having a half-life improvement factor (HIF) of >1.0 relative to a parent endoglucanase, e.g. an endoglucanase of SEQ ID NO:2.

In some aspects, the invention relates to a detergent composition comprising an isolated GH9 endoglucanase variant having activity on xanthan gum pretreated with xanthan lyase.

In some aspects, the detergent composition comprises a xanthan lyase variant comprising an alteration (e.g. a substitution, deletion or insertion, preferably a substitution) at one or more positions in two or more, three or more, four or more, five or all six regions selected from the group consisting of: (i) region 1 corresponding to amino acids 154 to 176 of SEQ ID NO:6; (ii) region 2 corresponding to amino acids 614 to 658 of SEQ ID NO:6; (iii) region 3 corresponding to amino acids 731 to 803 of SEQ ID NO:6; (iv) region 4 corresponding to amino acids 807 to 846 of SEQ ID NO:6; (v) region 5 corresponding to amino acids 872 to 885 of SEQ ID NO:6; and (vi) region 6 corresponding to amino acids 903 to 1004 of SEQ ID NO:6.

In some aspects, the detergent compositions comprise a xanthan lyase variant comprising an alteration (e.g. a substitution, deletion or insertion, preferably a substitution) at one or more positions in two or more regions selected from the group consisting of: (i) region 7 corresponding to amino acids 1 to 153 of SEQ ID NO:6; (ii) region 8 corresponding to amino acids 177 to 613 of SEQ ID NO:6; (iii) region 9 corresponding to amino acids 659 to 730 of SEQ ID NO:6; (iv) region 10 corresponding to amino acids 804 to 806 of SEQ ID NO:6; (v) region 11 corresponding to amino acids 847 to 871 of SEQ ID NO:6; (vi) region 12 corresponding to amino acids 886 to 902 of SEQ ID NO:6; and (vii) region 13 corresponding to amino acids 1005 to 1037 of SEQ ID NO:6.

In some aspects, the detergent composition comprises a xanthan lyase variant comprising an alteration (e.g. a substitution, deletion or insertion, preferably a substitution) at one or more positions in two or more, three or more, four or more, five or more, six or more, seven or more, eight or more, nine or more, ten or more, eleven or more, twelve or all thirteen regions selected from the group consisting of: (i) region 1 corresponding to amino acids 154 to 176 of SEQ ID NO:6; (ii) region 2 corresponding to amino acids 614 to 658 of SEQ ID NO:6; (iii) region 3 corresponding to amino acids 731 to 803 of SEQ ID NO:6; (iv) region 4 corresponding to amino acids 807 to 846 of SEQ ID NO:6; (v) region 5 corresponding to amino acids 872 to 885 of SEQ ID NO:6; (vi) region 6 corresponding to amino acids 903 to 1004 of SEQ ID NO:6; (vii) region 7 corresponding to amino acids 1 to 153 of SEQ ID NO:6; (iii) region 8 corresponding to amino acids 177 to 613 of SEQ ID NO:6; (xi) region 9 corresponding to amino acids 659 to 730 of SEQ ID NO:6; (x) region 10 corresponding to amino acids 804 to 806 of SEQ ID NO:6; (xi) region 11 corresponding to amino acids 847 to 871 of SEQ ID NO:6; (xii) region 12 corresponding to amino acids 886 to 902 of SEQ ID NO:6; and (xiii) region 13 corresponding to amino acids 1005 to 1037 of SEQ ID NO:6.

In some aspects of the detergent compoitions of the invention, the xanthan lyase variant comprises an alteration at one or more positions in at least one chelator-induced instability region as well as an alteration at one or more positions in at least one adjacent region. Thus, in some aspects the xanthan lyase variant, in addition to an alteration in one or more positions in at least one region selected from the group consisting of regions 1, 2, 3, 4, 5 and 6 as set forth above and elsewhere herein, further comprises an alteration (e.g. a substitution, deletion or insertion, preferably a substitution) at one or more positions in at least one region selected from the group consisting of: (vii) region 7 corresponding to amino acids 1 to 153 of SEQ ID NO:6; (viii) region 8 corresponding to amino acids 177 to 613 of SEQ ID NO:6; (ix) region 9 corresponding to amino acids 659 to 730 of SEQ ID NO:6; (x) region 10 corresponding to amino acids 804 to 806 of SEQ ID NO:6; (xi) region 11 corresponding to amino acids 847 to 871 of SEQ ID NO:6; (xii) region 12 corresponding to amino acids 886 to 902 of SEQ ID NO:6; and (xiii) region 13 corresponding to amino acids 1005 to 1037 of SEQ ID NO:6.

The xanthan lyase variant may e.g. comprise an alteration at one or more positions in each of one or more, two or more, three or more, four or more, five or more, six or all seven regions selected from the group consisting of regions 7, 8, 9, 10, 11, 12 and 13.

In a particular aspect, the xanthan lyase variant as described herein is one that does not comprise any amino acid alteration at a position outside of regions 7, 8, 9, 10, 11, 12 and 13. In this aspect, the xanthan lyase variant thus does not comprise any alteration (e.g. a substitution, deletion or insertion, preferably a substitution) in a region selected from the group consisting of: region 1 corresponding to amino acids 154 to 176 of SEQ ID NO:6, region 2 corresponding to amino acids 614 to 658 of SEQ ID NO:6, region 3 corresponding to amino acids 731 to 803 of SEQ ID NO:6, region 4 corresponding to amino acids 807 to 846 of SEQ ID NO:6, region 5 corresponding to amino acids 872 to 885 of SEQ ID NO:6, and region 6 corresponding to amino acids 903 to 1004 of SEQ ID NO:6. Alternatively, the xanthan lyase variant as described herein is one that does not comprise any amino acid alteration at a position outside of regions 1, 2, 3, 4, 5 and 6. In this aspect, the xanthan lyase variant thus does not comprise any alteration (e.g. a substitution, deletion or insertion, preferably a substitution) in a region selected from the group consisting of: region 7, region 8, region 9, region 10, region 11, region 12, and region 13. It is however preferred that the endoglucanase variant comprises at least one alteration in any of regions 1-6 and at least one alteration in any one regions 7-13.

In some aspects, the xanthan lyase variant as described herein is one that comprises an alteration (e.g. a substitution, deletion or insertion, preferably a substitution) at one or more positions in: (i) regions 3 and 5; (ii) regions 3, 5 and 12; (iii) regions 8, and 9; (iv) regions 2, 3, and 5; (v) regions 2, 3, 5, and 12; (vi) regions 3, 5, 8, 9, and 12; (vii) regions 2, 3, 5, 8, and 9; (viii) 3, 5, 8, 9, and 12; (ix) 2, 3, 5, 8, 9, and 12; (x) region 3; (xi) regions 3, 4 and 5; (xii) regions 7, 8 and 9; (xiii) regions 12 and 13; (xiv) regions 3, 4, 5, 8, 9, and 12; (xv) regions 8, 9, 12, and 13; (xvi) regions 7, 8, 9, 12, and 13; (xvii) regions 3, 4, 5, 7, 8, 9, and 12; and (xviii) regions 3, 4, 5, 7, 8, 9, 12, and 13, wherein said variant preferably has no alteration in the other regions besides those mentioned.

In some aspects, the detergent composition comprises a xanthan lyase variant having an alteration (e.g. a substitution, deletion or insertion, preferably a substitution) at one or more positions selected from the group consisting of positions: 155, 159, 620, 624, 626, 631, 635, 645, 649, 650, 656, 738, 745, 746, 748, 752, 753, 754, 757, 764, 769, 774, 775, 777, 779, 782, 785, 786, 789, 792, 796, 799, 800, 801, 819, 824, 843, 845, 875, 903, 911, 912, 915, 919, 921, 923, 925, 927, 928, 930, 932, 933, 941, 966, 967, 991 and 998 of SEQ ID NO:6.

In some aspects, the present invention relates to a detergent composition comprising a xanthan lyase variant having one or more substitutions selected from the group consisting of: Y155E, A159P, K620R, A624E, A626G, T631N, T631E, S635E, S635T, S635Q, A645S, T649V, T649K, T649R, Q650G, 1656V, G738L, K745R, F746L, L748T, P752R, P752K, G753E, G753Q, G753S, S754E, S754L, S754Q, S754R, S757D, S757P, S757E, P764V, P764K, A769D, A769T, A769R, A769S, A769E, A769Q, A769*, A774V, L775M, L775Y, L775A, L7751, L775S, L775F, L775Q, D777K, D777R, P779V, Y7821, A785T, N786K, G789R, K792W, K792Y, K792V, K792A, N796Q, A799H, V800P, D801G, K819R, K819T, K824R, A843P, D845E, 875T, K875E, T903A, T903Q, A911V, A911M, A911S, A912T, A9121, A912Y, T915Q, T915S, T915V, T915A, T919F, T919G, T919D, T921R, T921S, T923H, T923D, T925Q, T925D, T925R, T927K, D928W, Y930H, Y930L, Y930F, A932P, D933M, G941E, G941D, A966P, A967D, N991D and V998K.

In some aspects, the present invention relates to a detergent composition comprising a xanthan lyase variant having an alteration (e.g. a substitution, deletion or insertion, preferably a substitution) at one or more positions selected from the group consisting of: 624, 635, 649, 656, 738, 753, 754, 757, 769, 775, 777, 801, 843 and 875.

In some aspects, the present invention relates to a detergent composition comprising a xanthan lyase variant having one or more substitutions selected from the group consisting of: A624E, S635E, T649K, I656V, G738L, G753E, S754E, S754R, S757D, A769D, L775A, D777R, D801G, A843P and K875T.

In some aspects, the alteration at one or more positions in at least one region selected from the group consisting of regions 7, 8, 9, 10, 11, 12 and 13 is an alteration at one or more positions selected from the group consisting of: 9, 15, 18, 46, 58, 66, 89, 95, 100, 106, 109, 183, 188, 190, 203, 204, 221, 229, 234, 238, 240, 242, 243, 257, 258, 284, 291, 293, 316, 317, 320, 324, 329, 333, 339, 341, 352, 354, 360, 372, 377, 399, 400, 419, 440, 450, 451, 454, 458, 481, 492, 505, 533, 567, 568, 576, 578, 579, 582, 664, 672, 703, 722, 726, 727, 728, 851, 855, 856, 867, 887, 892, 899, 900, 901, 902, 915, 1008 and 1016 of SEQ ID NO:6. The xanthan lyase variant may e.g. comprise an alteration at two or more of these positions, e.g. at three, four, five, six, seven, eight, nine or ten of these positions.

In some aspects, the alteration at one or more positions in at least one region selected from the group consisting of regions 7, 8, 9, 10, 11, 12 and 13 comprises one or more substitutions selected from the group consisting of: K9R, N15T, T18D, L46D, A58L, S66H, Q89Y, K95E, S100D, N106Y, Q109R, Q109D, Q109F, Q109K, Q109A, K183Q, K183R, V188I, A190Q, A203P, K204R, A221P, E229N, E229S, E229V, I234V, I238W, I238L, I238M, I240W, N242S, G243V, Y257W, R258E, R284G, K291R, A293G, A293P, K316R, R317K, K320R, L324Q, K329R, K333R, L339M, I341P, V3521, S354P, K360G, K360R, Q372H, F377Y, N399K, K400R, F419Y, N440K, D450P, K451E, K451R, A454V, D458S, K481R, A492H, A492L, T505I, L5331, K567R, G568A, S578K, S578N, S578R, S579R, S579K, S582K, T664K, N672D, I703L, I722F, P726Q, T727P, M728V, S851F, K855R, E856D, P867S, K887R, N892Y, N892W, N892F, G899S, I900G, D901A, T902F, N1008D and K1016T of SEQ ID NO:6. The xanthan lyase variant may e.g. comprise two or more of these substitutions, e.g. three, four, five, six, seven, eight, nine or ten of said substititions.

In some aspects of the detergent compositions of the invention, the xanthan lyase variant comprises an alteration at one or more positions in at least one region selected from the group consisting of regions 1, 2, 3, 4, 5 and 6, and an alteration at one or more positions in at least one region selected from the group consisting of regions 7, 8, 9, 10, 11, 12 and 13. In one aspect, the variant comprises an alteration at one or more positions selected from the group consisting of positions 624, 631, 635, 649, 656, 738, 752, 753, 754, 757, 769, 775, 777, 800, 801, 843, 875, 911 and 915, and an alteration at one or more positions selected from the group consisting of positions 89, 100, 190, 229, 234, 352, 360, 399, 440, 458, 492, 567, 582, 664, 672, 703, 728, 892, 1008 and 1016 of SEQ ID NO:6.

The variant may, for example, comprise an alteration at two or more positions, e.g. three, four, five or more positions, selected from the group consisting of positions 624, 631, 635, 649, 656, 738, 752, 753, 754, 757, 769, 775, 777, 800, 801, 843, 875, 911 and 915, and an alteration at two or more positions, e.g. two, three, four, five or more positions, selected from the group consisting of positions 89, 100, 190, 229, 234, 352, 360, 399, 440, 458, 492, 567, 582, 664, 672, 703, 728, 892, 1008 and 1016 of SEQ ID NO:6.

Preferred positions for alteration in this aspect include one or more positions selected from the group consisting of positions 624, 635, 649, 656, 738, 753, 754, 757, 769, 775, 777, 801, 843 and 875, and one or more positions selected from the group consisting of positions 100, 190, 229, 234, 360, 399, 440, 458, 492, 567, 582, 672, 892 and 1008 of SEQ ID NO:6.

In one embodment of this aspect, the xanthan lyase variant comprises one or more substitutions selected from the group consisting of Q89Y, S100D, A190Q, E229S, I234V, V352I, K360G, N399K, N440K, D458S, A492H, A492L, K567R, S582K, T664K, N672D, I703L, M728V, N892Y N1008D and K1016T, and one or more substitutions selected from the group consisting of A624E, T631N, S635E, T649K, 1656V, G738L, P752K, P752R, G753E, S754E, S754R, S757D, A769D, L775A, D777R, V800P, D801G, A843P, K875T, A911V and T915A. The variant may, for example, comprise two or more substitutions, e.g. three, four, five or more substitutions, selected from the group consisting of Q89Y, S100D, A190Q, E229S, 1234V, V3521, K360G, N399K, N440K, D458S, A492H, A492L, K567R, S582K, T664K, N672D, 1703L, M728V, N892Y N1008D and K1016T, and two or more substitutions, e.g. three, four, five or more substiutitions, selected from the group consisting of A624E, T631N, S635E, T649K, 1656V, G738L, P752K, P752R, G753E, S754E, S754R, S757D, A769D, L775A, D777R, V800P, D801G, A843P, K875T, A911V and T915A. Preferred substitutions in this embodiment include one or more substitutions selected from the group consisting of S100D, A190Q, E229S, 1234V, K360G, N399K, N440K, D458S, A492H, K567R, S582K, N672D, N892Y and N1008D, and one or more substitutions selected from the group consisting of A624E, S635E, T649K, 1656V, G738L, G753E, S754E, S754R, S757D, A769D, L775A, D777R, D801G, A843P and K875T.

Non-limiting examples of such variants include:
A190Q, E229S, S635E, T649K, 1656V, N672D, I703L, G753E, S754E, S757D, A769D, L775A, D801G, K875T, N892Y, N1008D
E229S, S635E, T649K, I656V, N672D, I703L, G753E, S754E, A769D, L775A, D801G, K875T, N892Y, N1008D
E229S, V3521, S635E, T649K, 1656V, N672D, G753E, S754E, A769D, L775A, V800P,
E229S, K360G, D458S, S582K, N672D, G753E, S754E, A769D, L775A, D801G, K875T, N892Y, N1008D
S100D, E229S, K360G, D458S, S582K, T664K, N672D, G753E, S754E, S757D, A769D, L775A, D801G, A843P, K875T, N892Y, A911V, N1008D, K1016T
E229S, I234V, S582K, N672D, G753E, S754E, A769D, L775A, V800P, D801G, K875T, N892Y
Q89Y, E229S, N440K, S582K, A624E, N672D, G753E, S754E, A769D, L775A, D801G, K875T, N892Y
E229S, S635E, T649K, I656V, N672D, P752K, G753E, A769D, L775A, D801G, A843P, K875T, N892Y
E229S, S635E, T649K, I656V, N672D, G753E, S754E, S757D, A769D, L775A, D801G, K875T, N892Y
E229S, N440K, S582K, N672D, G753E, S754E, A769D, L775A, D801G, A843P, K875T, N892Y, N1008D
E229S, N440K, S582K, A624E, N672D, G753E, S754E, A769D, L775A, V800P, D801G, K875T, N892Y
A190Q, E229S, S635E, T649K, I656V, N672D, P752K, G753E, A769D, L775A, D801G, A843P, K875T, N892Y
A190Q, E229S, S582K, N672D, G753E, S754E, A769D, L775A, D801G, K875T, N892Y
E229S, N440K, S582K, N672D, P752R, G753E, S754E, S757D, A769D, L775A, D801G, K875T, N892Y, N1008D
E229S, S582K, S635E, N672D, P752R, G753E, S754E, A769D, L775A, D801G, K875T, N892Y, N1008D
A190Q, E229S, N440K, S582K, A624E, S635E, N672D, G753E, S754E, A769D, L775A, D801G, K875T, N892Y
E229S, I234V, A492L, S582K, N672D, G753E, S754E, A769D, L775A, D801G, K875T, N892Y
A190Q, E229S, K360G, D458S, S582K, T664K, N672D, G753E, S754E, A769D, L775A, D801G, K875T, N892Y, N1008D
S100D, E229S, K360G, D458S, S582K, N672D, G753E, S754E, S757D, A769D, L775A, D801G, A843P, K875T, N892Y, T915A, N1008D

E229S, N440K, S582K, A624E, S635E, N672D, G738L, G753E, S754E, S757D, A769D, L775A, D801G, K875T, N892Y
S100D, E229S, K360G, D458S, S582K, N672D, G753E, S754E, A769D, L775A, D801G, K875T, N892Y, N1008D
A190Q, E229S, D458S, T631N, N672D, G753E, S754E, A769D, L775A, D801G, A843P, K875T, N892Y
A190Q, E229S, K360G, D458S, S582K, N672D, G753E, S754E, A769D, L775A, D801G, K875T, N892Y, N1008D
E229S, S635E, T649K, 1656V, N672D, G753E, S754R, S757D, A769D, L775A, D801G, A843P, K875T, N892Y
E229S, D458S, S582K, T631N, S635E, N672D, M728V, G753E, S754E, S757D, A769D, L775A, D801G, K875T, N892Y
A190Q, E229S, K360G, D458S, S582K, N672D, G753E, S754E, S757D, A769D, L775A, D801G, K875T, N892Y, N1008D
E229S, A492L, S635E, T649K, 1656V, N672D, G753E, S757D, A769D, L775A, D801G, K875T, N892Y
S100D, A190Q, E229S, K360G, D458S, S582K, N672D, G753E, S754E, A769D, L775A, D801G, K875T, N892Y, N1008D
A190Q, E229S, I234V, S582K, N672D, G753E, S754E, S757D, A769D, L775A, D801G, K875T, N892Y
E229S, N399K, D458S, A492H, K567R, S582K, S635E, T649K, N672D, G753E, S754E, A769D, L775A, D777R, D801G, K875T, N892Y
E229S, D458S, A492L, T631N, N672D, G753E, S754E, S757D, A769D, L775A, D801G, K875T, N892Y
E229S, D458S, A492H, K567R, S582K, S635E, N672D, G753E, S754E, A769D, L775A, D777R, D801G, K875T, N892Y
S100D, E229S, K360G, D458S, S582K, N672D, G753E, S754E, S757D, A769D, L775A, D801G, A843P, K875T, N892Y, N1008D
E229S, N399K, D458S, K567R, S582K, S635E, N672D, G753E, S754E, A769D, L775A, D777R, D801G, K875T, N892Y.

In some aspects, the present invention relates to detergent compositions comprising a xanthan lyase variant having an alteration (e.g. a substitution, deletion or insertion, preferably a substitution) at one or more positions selected from the group consisting of positions: 9, 15, 46, 58, 66, 89, 95, 100, 106, 109, 183, 188, 190, 203, 204, 221, 229, 234, 238, 240, 242, 243, 257, 258, 291, 293, 316, 320, 324, 329, 333, 339, 341, 352, 354, 360, 377, 400, 419, 450, 451, 454, 481, 492, 567, 568, 578, 579, 664, 672, 855, 887 and 892 of SEQ ID NO:6.

In some aspects, the present invention relates to detergent compositions comprising a xanthan lyase variant having one or more substitutions selected from the group consisting of: K9R, N15T, L46D, A58L, S66H, Q89Y, K95E, S100D, N106Y, Q109R, Q109D, Q109F, Q109K, Q109A, K183Q, K183R, V1881, A190Q, A203P, K204R, A221P, E229N, E229S, I234V, I238W, I238L, I238M, I240W, N242S, G243V, Y257W, R258E, K291R, A293G, A293P, K316R, K320R, L324Q, K329R, K333R, L339M, I341P, V3521, S354P, K360R, F377Y, K400R, F419Y, D450P, K451E, K451R, A454V, K481R, A492L, K567R, G568A, S578K, S578R, S579R, S579K, T664K, N672D, K855R, K887R, N892Y, N892W and N892F.

In some aspects, the present invention relates to a detergent composition comprising a xanthan lyase variant having an alteration (e.g. a substitution, deletion or insertion, preferably a substitution) in the positions selected from the group consisting of positions: 190, 229, 234, 440, 582, 624, 631, 635, 672, 703, 738, 752, 753, 754, 757, 769, 775, 801, 875, 892, and any combination thereof, preferably 229+672+752+753+769+775+801+875+892; 229+672+753+754+769+775+801+875+892;229+672+752+753+754+769+775+801+875+892; 190+229+234+624+672+753+754+769+775+801+875; 190+229+631+672+703+752+753+769+775+801+875; 190+229+234+582+672+753+754+757+769+775+801+875+892; 229+440+582+624+635+672+738+753+754+757+769+775+801+875+892; or 100+229+360+458+582+672+753+754+757+769+775+801+843+875+892+1008 of SEQ ID NO:6.

In some aspects, the present invention relates to a detergent composition comprising a xanthan lyase variant having an alteration (e.g. a substitution, deletion or insertion, preferably a substitution) selected from the group consisting of the following alterations: E229N+N672D+P752K+G753E+A769D+L775A+D801G+K875T+N892Y; E2295+N672D+G753E+5754E+A769D+L775A+D801G+K875T+N892Y; E2295+N672D+P752R+G753E+5754E+A769D+L775A+D801G+K875T+N892Y; A190Q+E229S+1234V+A624E+N672D+G753E+S754E+A769D+L775A+D801G+K875T; A190Q+E229S+T631N+N672D+1703L+P752K+G753E+A769D+L775A+D801G+K875T; A190Q+E229S+1234V+S582K+N672D+G753E+S754E+S757D+A769D+L775A+D801G+K875T+N89 2Y; E229S+N440K+S582K+A624E+S635E+N672D+G738L+G753E+S754E+S757D+A769D+L775A+D80 1G+K875T+N892Y; or S100D+E229S+K360G+D458S+S582K+N672D+G753E+S754E+S757D+A769D+L775A+D801G+A8 43P+K875T+N892Y+N1008D of SEQ ID NO:6.

In some aspects, the present invention relates to a detergent composition comprising a xanthan lyase variant having activity on xanthan gum; preferably said activity comprises xanthan lyase EC 4.2.2.12 activity, further preferably said activity is xanthan lyase EC 4.2.2.12 activity.

In some aspects, the present invention relates to a detergent composition comprising a xanthan lyase variant having an improved stability in said detergent composition compared to a parent xanthan lyase (e.g. with SEQ ID NO:6).

In some aspects, the present invention relates to a detergent composition comprising a xanthan lyase variant having a half-life improvement factor (HIF) of >1.0 relative to a parent xanthan lyase.

In some aspects, the invention relates to a detergent composition comprising an isolated xanthan lyase variant having activity on xanthan gum according to the invention.

In some aspects, the present invention relates to a detergent composition, as defined herein, comprising an endoglucanase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% sequence identity to SEQ ID NO:2 and having an alteration (e.g. a substitution, deletion or insertion, preferably a substitution) in the positions selected from the group consisting of positions: 559+579+697; 512+559+579+697; 18+71+186+408+579+602+651+688+756; 18+189+408+559+579+688+697+756+921+934; 313+488; 880+905+921+934; 302+313+408+579+602+651+697+880+921+934; or 216+313+408+476+579+602+638+651+697+719+880+887+921+934 of SEQ ID NO:2. Preferably, the endoglucanase variant has an alteration selected from the group consisting of the following alterations: A559N+Y579W+T697G; K512P+A559N+Y579W+T697G; N18G+A71E+A186P+E408D+Y579W+1602T+A651P+A688G+V756Y; N18G+N189K+E408D+A559N+Y579W+A688G+T697G+V756Y+K921R+Y934G; S313D+E408D; R880K+N905D+K921R+Y934G; I302D+S313D+E408D+Y579W+1602T+A651P+T697G+R880K+K921R+Y934G; and N216Q+S313D+E408D+D476R+Y579W+I602T+F638N+A651P+T697G+W719R+R880K+T887K+K9 21R+Y934G of SEQ ID NO:2. In a particular aspect, the endoglucanase variant has besides the aforementioned alterations no further alterations relative to the parent enzyme of SEQ ID NO:2, i.e. the remaining sequence is identical to SEQ ID NO:2.

In some aspects, the present invention relates to a detergent composition, as defined herein, comprising a xanthan lyase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% sequence identity to SEQ ID NO:6 and having an alteration (e.g. a substitution, deletion or insertion, preferably a substitution) in the positions selected from the group consisting of positions: 229+672+752+753+769+775+801+875+892; 229+672+753+754+769+775+801+875+892;229+672+752+753+754+769+775+801+875+892; 190+229+234+624+672+753+754+769+775+801+875; 190+229+631+672+703+752+753+769+775+801+875; 190+229+234+582+672+753+754+757+769+775+801+875+892; 229+440+582+624+635+672+738+753+754+757+769+775+801+875+892; or 100+229+360+458+582+672+753+754+757+769+775+801+843+875+892+1008 of SEQ ID NO:6. Preferably, the xanthan lyase variant has an alteration selected from the group consisting of the following alterations: E229N+N672D+P752K+G753E+A769D+L775A+D801G+K875T+N892Y; E2295+N672D+G753E+5754E+A769D+L775A+D801G+K875T+N892Y; E2295+N672D+P752R+G753E+5754E+A769D+L775A+D801G+K875T+N892Y; A190Q+E229S+1234V+A624E+N672D+G753E+S754E+A769D+L775A+D801G+K875T; A190Q+E229S+T631N+N672D+I703L+P752K+G753E+A769D+L775A+D801G+K875T; A190Q+E229S+1234V+S582K+N672D+G753E+S754E+S757D+A769D+L775A+D801G+K875T+N89 2Y;

E229S+N440K+S582K+A624E+S635E+N672D+G738L+G753E+S754E+S757D+A769D+L775A+D80 1G+K875T+N892Y; or S100D+E229S+K360G+D458S+S582K+N672D+G753E+S754E+S757D+A769D+L775A+D801G+A8 43P+K875T+N892Y+N1008D of SEQ ID NO:6. In a particular aspect, the xanthan lyase variant has besides the afore-mentioned alterations no further alterations relative to the parent enzyme of SEQ ID NO:6, i.e. the remaining sequence is identical to SEQ ID NO:6.

In various embodiments, the preferred endoglucanase variants are combined with the preferred xanthan lyase variants. In some aspects, the detergent composition thus comprises

(A) an endoglucanase variant selected from those having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% sequence identity to SEQ ID NO:2 and having an alteration (e.g. a substitution, deletion or insertion, preferably a substitution) in the positions selected from the group consisting of positions: 559+579+697; 512+559+579+697; 18+71+186+408+579+602+651+688+756; 18+189+408+559+579+688+697+756+921+934; 313+488; 880+905+921+934; 302+313+408+579+602+651+697+880+921+934; or 216+313+408+476+579+602+638+651+697+719+880+887+921+934 of SEQ ID NO:2, preferably having an alteration selected from the group consisting of the following alterations: (A1) A559N+Y579W+T697G; (A2) K512P+A559N+Y579W+T697G; (A3) N18G+A71E+A186P+E408D+Y579W+I602T+A651P+A688G+V756Y; (A4) N18G+N189K+E408D+A559N+Y579W+A688G+T697G+V756Y+K921R+Y934G; (A5) S313D+E408D; (A6) R880K+N905D+K921R+Y934G; (A7) I302D+S313D+E408D+Y579W+I602T+A651P+T697G+R880K+K921R+Y934G; and (A8) N216Q+S313D+E408D+D476R+Y579W+I602T+F638N+A651P+T697G+W719R+R880K+T887K+K9 21R+Y934G of SEQ ID NO:2; and

(B) a xanthan lyase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% sequence identity to SEQ ID NO:6 and having an alteration (e.g. a substitution, deletion or insertion, preferably a substitution) in the positions selected from the group consisting of positions: 229+672+752+753+769+775+801+875+892; 229+672+753+754+769+775+801+875+892;229+672+752+753+754+769+775+801+875+892; 190+229+234+624+672+753+754+769+775+801+875; 190+229+631+672+703+752+753+769+775+801+875; 190+229+234+582+672+753+754+757+769+775+801+875+892; 229+440+582+624+635+672+738+753+754+757+769+775+801+875+892; or 100+229+360+458+582+672+753+754+757+769+775+801+843+875+892+1008 of SEQ ID NO:6, preferably having an alteration selected from the group consisting of the following alterations: (B1) E229N+N672D+P752K+G753E+A769D+L775A+D801G+K875T+N892Y; (B2) E2295+N672D+G753E+5754E+A769D+L775A+D801G+K875T+N892Y; (B3) E229S+N672D+P752R+G753E+S754E+A769D+L775A+D801G+K875T+N892Y; (B4) A190Q+E229S+1234V+A624E+N672D+G753E+S754E+A769D+L775A+D801G+K875T; (B5) A190Q+E229S+T631N+N672D+1703L+P752K+G753E+A769D+L775A+D801G+K875T; (B6) A190Q+E229S+1234V+S582K+N672D+G753E+S754E+S757D+A769D+L775A+D801G+K875T+N89 2Y; (B7) E229S+N440K+S582K+A624E+S635E+N672D+G738L+G753E+S754E+S757D+A769D+L775A+D80 1G+K875T+N892Y; and (B8)

S100D+E229S+K360G+D458S+S582K+N672D+G753E+S754E+S757D+A769D+L775A+D801G+A8 43P+K875T+N892Y+N1008D of SEQ ID NO:6.

In some aspects, the endoglucanase and/or the xanthan lyase variant do not comprise any further substitution besides those explicitly mentioned above, i.e. the remainder of the sequence is identical to that of the parent enzyme as set forth in SEQ ID NO:2 and SEQ ID NO:6, respectively.

In some aspects, in the detergent compositions of the invention the endoglucanase variant A1, as defined above, can be combined with any one of the xanthan lyase variants B1, B2, B3, B4, B5, B6, B7 and B8, as defined above, wherein the respective enzymes preferably have at least 90%, preferably at least 91%, 92%, 93%, 94%, 95%, 96% or 97% sequence identity to SEQ ID NO:2 and SEQ ID NO:6, respectively, preferably are identical to their respective parent sequence with the exception of the substitutions explicitly mentioned herein.

In some aspects, in the detergent compositions of the invention the endoglucanase variant A2, as defined above, can be combined with any one of the xanthan lyase variants B1, B2, B3, B4, B5, B6, B7 and B8, as defined above, wherein the respective enzymes preferably have at least 90%, preferably at least 91%, 92%, 93%, 94%, 95%, 96% or 97% sequence identity to SEQ ID NO:2 and SEQ ID NO:6, respectively, preferably are identical to their respective parent sequence with the exception of the substitutions explicitly mentioned herein.

In some aspects, in the detergent compositions of the invention the endoglucanase variant A3, as defined above, can be combined with any one of the xanthan lyase variants B1, B2, B3, B4, B5, B6, B7 and B8, as defined above, wherein the respective enzymes preferably have at least 90%, preferably at least 91%, 92%, 93%, 94%, 95%, 96% or 97% sequence identity to SEQ ID NO:2 and SEQ ID NO:6, respectively, preferably are identical to their respective parent sequence with the exception of the substitutions explicitly mentioned herein.

In some aspects, in the detergent compositions of the invention the endoglucanase variant A4, as defined above, can be combined with any one of the xanthan lyase variants B1, B2, B3, B4, B5, B6, B7 and B8, as defined above, wherein the respective enzymes preferably have at least 90%, preferably at least 91%, 92%, 93%, 94%, 95%, 96% or 97% sequence identity to SEQ ID NO:2 and SEQ ID NO:6, respectively, preferably are identical to their respective parent sequence with the exception of the substitutions explicitly mentioned herein.

In some aspects, in the detergent compositions of the invention the endoglucanase variant A5, as defined above, can be combined with any one of the xanthan lyase variants B1, B2, B3, B4, B5, B6, B7 and B8, as defined above, wherein the respective enzymes preferably have at least 90%, preferably at least 91%, 92%, 93%, 94%, 95%, 96% or 97% sequence identity to SEQ ID NO:2 and SEQ ID NO:6, respectively, preferably are identical to their respective parent sequence with the exception of the substitutions explicitly mentioned herein.

In some aspects, in the detergent compositions of the invention the endoglucanase variant A6, as defined above, can be combined with any one of the xanthan lyase variants B1, B2, B3, B4, B5, B6, B7 and B8, as defined above, wherein the respective enzymes preferably have at least 90%, preferably at least 91%, 92%, 93%, 94%, 95%, 96% or 97% sequence identity to SEQ ID NO:2 and SEQ ID NO:6, respectively, preferably are identical to their respective parent sequence with the exception of the substitutions explicitly mentioned herein.

In some aspects, in the detergent compositions of the invention the endoglucanase variant A7, as defined above, can be combined with any one of the xanthan lyase variants B1, B2, B3, B4, B5, B6, B7 and B8, as defined above, wherein the respective enzymes preferably have at least 90%, preferably at least 91%, 92%, 93%, 94%, 95%, 96% or 97% sequence identity to SEQ ID NO:2 and SEQ ID NO:6, respectively, preferably are identical to their respective parent sequence with the exception of the substitutions explicitly mentioned herein.

In some aspects, in the detergent compositions of the invention the endoglucanase variant A8, as defined above, can be combined with any one of the xanthan lyase variants B1, B2, B3, B4, B5, B6, B7 and B8, as defined above, wherein the respective enzymes preferably have at least 90%, preferably at least 91%, 92%, 93%, 94%, 95%, 96% or 97% sequence identity to SEQ ID NO:2 and SEQ ID NO:6, respectively, preferably are identical to their respective parent sequence with the exception of the substitutions explicitly mentioned herein.

In some aspects, preferred combinations of endoglucanase variants and xanthan lyase variants of the invention, are the combinations disclosed in Tables 34-36.

In some aspects, the present invention relates to a detergent composition additionally comprising one or more further detergent components, preferably a surfactant.

In some aspects, the present invention relates to use of a composition of the present invention, wherein said use is selected from the group consisting of: use for degrading xanthan gum and use in a cleaning process, such as laundry or hard surface cleaning such as dish wash.

In some aspects, the present invention further relates to the use of a detergent composition of the invention for degrading xanthan gum, for washing or cleaning textiles and/or hard surfaces, such as dish wash, wherein the composition has an enzyme detergency benefit.

In some aspects, the present invention also relates to methods of degrading xanthan gum using detergent compositions of the present invention, wherein xanthan gum is on the surface of a hard surface or textile.

### OVERVIEW OF SEQUENCE LISTING

SEQ ID NO:1 is the DNA sequence of the parent mature endoglucanase from a strain of a Paenibacillus sp.

SEQ ID NO:2 is the amino acid sequence of mature polypeptide encoded by SEQ ID NO:1.

SEQ ID NO:3 is the DNA sequence of the alpha-amylase secretion signal from Bacillus licheniformis.

SEQ ID NO:4 is the amino acid sequence of the alpha-amylase secretion signal from Bacillus licheniformis.

SEQ ID NO:5 is the DNA sequence of the parent mature xanthan lyase from a strain of a Paenibacillus sp.

SEQ ID NO:6 is the amino acid sequence of the mature polypeptide encoded by SEQ ID NO:5.

Definitions

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Cleaning or Detergent Application: the term “cleaning or detergent application” means applying the endoglucanase of the application in any composition for the purpose of cleaning or washing, by hand, machine or automated, a hard surface or a textile.

Cleaning Composition: the term “cleaning composition” refers to compositions that find use in the removal of undesired compounds from items to be cleaned, such as textiles, dishes, and hard surfaces. The terms encompass any materials/compounds selected for the particular type of cleaning composition desired and the form of the product (e.g. liquid, gel, powder, granulate, paste, or spray compositions) and includes, but is not limited to, detergent compositions (e.g. liquid and/or solid laundry detergents and fine fabric detergents; hard surface cleaning formulations, such as for glass, wood, ceramic and metal counter tops and windows; carpet cleaners; oven cleaners; fabric fresheners; fabric softeners; and textile and laundry pre-spotters, as well as dish wash detergents). In addition to the endoglucanase and xanthan lyase, the detergent formulation may contain one or more additional enzymes (such as xanthan lyases, proteases, amylases, lipases, cutinases, cellulases, endoglucanases, xyloglucanases, pectinases, pectin lyases, xanthanases, peroxidaes, haloperoxygenases, catalases and mannanases, or any mixture thereof), and/or components such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti corrosion agents, enzyme inhibitors or stabilizers, enzyme activators, transferase(s), hydrolytic enzymes, oxido reductases, bluing agents and fluorescent dyes, antioxidants, and solubilizers.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Colour clarification: During washing and wearing loose or broken fibers can accumulate on the surface of the fabrics. One consequence can be that the colours of the fabric appear less bright or less intense because of the surface contaminations. Removal of the loose or broken fibers from the textile will partly restore the original colours and looks of the textile. By the term “colour clarification”, as used herein, is meant the partial restoration of the initial colours of textile.

Control sequences: The term “control sequences” means nucleic acid sequences necessary for expression of a polynucleotide encoding a mature polypeptide of the present invention. Each control sequence may be native (i.e. from the same gene) or foreign (i.e. from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Corresponding to: The term “corresponding to” as used herein, refers to a way of determining the specific amino acid of a sequence wherein reference is made to a specific amino acid sequence. E.g. for the purposes of the present invention, when references are made to specific amino acid positions, the skilled person would be able to align another amino acid sequence to said amino acid sequence that reference has been made to, in order to determine which specific amino acid may be of interest in said another amino acid sequence. Alignment of another amino acid sequence with e.g. the sequence as set forth in SEQ ID NO:2, or any other amino acid sequence listed herein, has been described elsewhere herein. Alternative alignment methods may be used, and are well-known for the skilled person.

Degrading xanthan gum and xanthan gum degrading activity: The terms “degrading xanthan gum” and “xanthan gum degrading activity” are used interchangebly and are defined as the depolymerization, degradation or breaking down of xanthan gum into smaller components. The degradation of xanthan gum can either be the removal of one or more side chain saccharides, the cutting of the backbone of xanthan gum into smaller components or the removal of one or more side chain saccharides and the cutting of the backbone of xanthan gum into smaller components. A preferred assay for measuring degradation of xanthan gum is the reducing sugar assay as described in Examples 3 and 7 herein. Non-limiting examples of the xanthan gum degrading activity include endoglucanase EC 3.2.1.4 activity and/or xanthan lyase EC 4.2.2.12 activity.

Detergent component: the term “detergent component” is defined herein to mean the types of chemicals which can be used in detergent compositions. Examples of detergent components are surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors or stabilizers, enzyme activators, antioxidants, and solubilizers. The detergent composition may comprise of one or more of any type of detergent component.

Detergent composition: the term “detergent composition” refers to compositions that find use in the removal of undesired compounds from items to be cleaned, such as textiles, dishes, and hard surfaces. The detergent composition may be used to e.g. clean textiles, dishes and hard surfaces for both household cleaning and industrial cleaning. The terms encompass any materials/compounds selected for the particular type of cleaning composition desired and the form of the product (e.g. liquid, gel, powder, granulate, paste, or spray compositions) and includes, but is not limited to, detergent compositions (e.g. liquid and/or solid laundry detergents and fine fabric detergents; hard surface cleaning formulations, such as for glass, wood, ceramic and metal counter tops and windows; carpet cleaners; oven cleaners; fabric fresheners; fabric softeners; and textile and laundry pre-spotters, as well as dish wash detergents). In addition to containing a GH9 endoglucanase as described herein and/or xanthan lyase, the detergent formulation may contain one or more additional enzymes (such as xanthan lyases, proteases, amylases, lipases, cutinases, cellulases, endoglucanases, xyloglucanases, pectinases, pectin lyases, xanthanases, peroxidaes, haloperoxygenases, catalases and mannanases, or any mixture thereof), and/or components such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti corrosion agents, enzyme inhibitors or stabilizers, enzyme activators, transferase(s), hydrolytic enzymes, oxido reductases, bluing agents and fluorescent dyes, antioxidants, and solubilizers.

Dish wash: The term “dish wash” refers to all forms of washing dishes, e.g. by hand or automatic dish wash. Washing dishes includes, but is not limited to, the cleaning of all forms of crockery such as plates, cups, glasses, bowls, all forms of cutlery such as spoons, knives, forks and serving utensils as well as ceramics, plastics, metals, china, glass and acrylics.

Dish washing composition: The term “dish washing composition” refers to all forms of compositions for cleaning hard surfaces. The present invention is not restricted to any particular type of dish wash composition or any particular detergent.

Endoglucanase: The term “endoglucanase” or “EG” means an endo-1,4-(1,3;1,4)-beta-D-glucan 4-glucanohydrolase (EC 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans, xyloglucans, xanthans and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). A preferred assay for measuring endoglucanase activity is the reducing sugar assay as described in Examples 3 and 7 herein. Non-limiting examples of endoglucanases include the mature parent endoglucanase having SEQ ID NO:2.

Enzyme detergency benefit: The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and or cleaning, prevention or reduction of redeposition of soils released in the washing process an effect that also is termed anti-redeposition, restoring fully or partly the whiteness of textiles, which originally were white but after repeated use and wash have obtained a greyish or yellowish appearance an effect that also is termed whitening. Textile care benefits, which are not directly related to catalytic stain removal or prevention of redeposition of soils are also important for enzyme detergency benefits. Examples of such textile care benefits are prevention or reduction of dye transfer from one fabric to another fabric or another part of the same fabric an effect that is also termed dye transfer inhibition or anti-backstaining, removal of protruding or broken fibers from a fabric surface to decrease pilling tendencies or remove already existing pills or fuzz an effect that also is termed anti-pilling, improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching component such as hydrogen peroxide or other peroxides.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide having one or more (e.g. several) amino acids absent from the amino and/or carboxyl terminus of a mature polypeptide; wherein the fragment has endoglucanase activity. In one aspect, a fragment contains at least 85%, 86%, 87%, 88%, 89%, 90%, 91%, 92%, 93%, 94% or 95% of the number of amino acids of the mature polypeptide.

Endoglucanase variant having activity on xanthan gum pretreated with xanthan lyase: The term “Endoglucanase variant having activity on xanthan gum pretreated with xanthan lyase” or an “endoglucanase having activity on xanthan gum pretreated with xanthan lyase and belonging to the GH9 class of glycosyl hydrioases” is defined as a polypeptide comprising a domain belonging to the GH9 class of glycosyl hydrolases, and having activity (e.g. enzymatic activity, xanthan degrading activity, endoglucanase EC 3.2.1.4 activity) on xanthan gum pretreated with xanthan lyase. A preferred assay for measuring activity on xanthan gum pretreated with xanthan lyase is disclosed in Example 3 herein.

Xanthan lyase variant having activity on xanthan gum: The term “Xanthan lyase variant having activity on xanthan gum” is defined as a polypeptide that cleaves the □-D-mannosyl-□-D-1,4-glucuronosyl bond of xanthan (e.g. xanthan lyase EC 4.2.2.12 activity). A preferred assay for measuring activity on xanthan gum is disclosed in Example 7 herein. Examples of the xanthan lyase variants having activity on xanthan gum, are xanthan lyase polypeptides as such. Thus, polypeptides that that cleaves the □-D-mannosyl-□-D-1,4-glucuronosyl bond of xanthan.

Half-life: the term “half-life” is the time it takes for an enzyme to lose half of its enzymatic activity under a given set of conditions. It is denoted as T_{1/2} and is measured in hours (h). Half-lifes can be calculated at a given detegent concentration and storage temperature for a Wild-type control and/or variants, as the degradation follows an exponential decay and the incubation time (hours) is known, i.e. according to the following formulas:

T½ (variant)=(Ln (0.5)/Ln (RA-variant/100))*Time

T½ (wild-type)=(Ln (0.5)/Ln (RA-wild-type/100))*Time

Wherein ‘RA’ is the residual activity in percent and ‘Time’ is the incubation time in hours.

Half-life improvement factor: the term “Half-life improvement factor” or “HIF” is the improvement of half-life of a variant compared to the parent polypeptide, such as the parent endoglucanase. A half-life improvement factor (HIF) under a given set of storage conditions (detergent concentration and temperature) can be calculated as: HIF=T½ (variant)/T½ (wild-type) where the wild-type (wt) is incubated under the same storage condition (detergent concentration and incubation temperature) as the variant. In the cases where the difference in stability between wild-type and variant is too big to accurately assess half-life for both wild-type and variant using the same incubation time, the incubation time for wild-type and variant is different e.g. 1 h for wild-type and >100 h for more stable variants. The half-life improvement factor may also be calculated based on the half-life of a parent xanthan lyase (see the definition of “parent” below) that is not necessarily a wild-type. Preferred ways of calculating HIF are also described in Examples 3 and 7 herein.

Hard surface cleaning: The term “Hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, and cutlery such as spoons, knives, forks, serving utensils, ceramics, plastics, metals, china, glass and acrylics.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Improved property: The term “improved property” means a characteristic associated with a variant that is improved compared to the parent. Such improved properties include, but are not limited to, catalytic efficiency, catalytic rate, chemical stability, oxidation stability, pH activity, pH stability, specific activity, stability under storage conditions, chelator stability, substrate binding, substrate cleavage, substrate specificity, substrate stability, surface properties, thermal activity, and thermostability.

Improved wash performance: The term “improved wash performance” is defined herein as a (variant) enzyme (also a blend of enzymes, not necessarily only variants but also backbones, and in combination with certain cleaning composition etc.) displaying an alteration of the wash performance of a protease variant relative to the wash performance of the parent protease variant e.g. by increased stain removal. The term “wash performance” includes wash performance in laundry but also e.g. in dish wash.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g. multiple copies of a gene encoding the substance; use of a stronger promoter than the promoter naturally associated with the gene encoding the substance). An isolated substance may be present in a fermentation broth sample.

Laundering: The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 1 to 1055 of SEQ ID NO:2 or amino acids 1 to 1037 of SEQ ID NO:6.

It is known in the art that a host cell may produce a mixture of two of more different mature polypeptides (i.e. with a different C-terminal and/or N-terminal amino acid) expressed by the same polynucleotide. It is also known in the art that different host cells process polypeptides differently, and thus, one host cell expressing a polynucleotide may produce a different mature polypeptide (e.g. having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having enzymatic activity such as activity on xanthan gum pretreated with xanthan lyase or xanthan lyase activity. In one aspect, the mature polypeptide coding sequence is nucleotides 1 to 3165 of SEQ ID NO:1 or nucleotides 1 to 3111 of SEQ ID NO:5.

Mutant: The term “mutant” means a polynucleotide encoding a variant.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent: The term “parent” or “parent endoglucanase” means any polypeptide with endoglucanase activity to which an alteration is made to produce the enzyme variants of the present invention. In one aspect, the parent is an endoglucanase having the identical amino acid sequence of the variant, but not having the alterations at one or more of the specified positions. It will be understood, that the expression “having identical amino acid sequence” relates to 100% sequence identity. Non-limiting examples of parent endoglucanases include the mature parent endoglucanase having SEQ ID NO:2.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment—Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides x 100)/(Length of Alignment−Total Number of Gaps in Alignment)

Stringency conditions: The different strigency conditions are defined as follows.

The term “very low stringency conditions” means for probes of at least 100 nucleotides in length, prehybridization and hybridization at 42° C. in 5× SSPE, 0.3% SDS, 200 mg/mL sheared and denatured salmon sperm DNA, and 25% formamide, following standard Southern blotting procedures for 12 to 24 h. The carrier material is finally washed three times each for 15 minutes using 2× SSC, 0.2% SDS at 45° C.

The term “low stringency conditions” means for probes of at least 100 nucleotides in length, prehybridization and hybridization at 42° C. in 5× SSPE, 0.3% SDS, 200 mg/mL sheared and denatured salmon sperm DNA, and 25% formamide, following standard Southern blotting procedures for 12 to 24 h. The carrier material is finally washed three times each for 15 minutes using 2× SSC, 0.2% SDS at 50° C.

The term “medium stringency conditions” means for probes of at least 100 nucleotides in length, prehybridization and hybridization at 42° C. in 5× SSPE, 0.3% SDS, 200 mg/mL sheared and denatured salmon sperm DNA, and 35% formamide, following standard Southern blotting procedures for 12 to 24 h. The carrier material is finally washed three times each for 15 minutes using 2× SSC, 0.2% SDS at 55° C.

The term “medium-high stringency conditions” means for probes of at least 100 nucleotides in length, prehybridization and hybridization at 42° C. in 5× SSPE, 0.3% SDS, 200 mg/mL sheared and denatured salmon sperm DNA, and 35% formamide, following standard Southern blotting procedures for 12 to 24 h. The carrier material is finally washed three times each for 15 minutes using 2× SSC, 0.2% SDS at 60° C.

The term “high stringency conditions” means for probes of at least 100 nucleotides in length, prehybridization and hybridization at 42° C. in 5× SSPE, 0.3% SDS, 200 mg/mL sheared and denatured salmon sperm DNA, and 50% formamide, following standard Southern blotting procedures for 12 to 24 h. The carrier material is finally washed three times each for 15 minutes using 2× SSC, 0.2% SDS at 65° C.

The term “very high stringency conditions” means for probes of at least 100 nucleotides in length, prehybridization and hybridization at 42° C. in 5× SSPE, 0.3% SDS, 200 mg/mL sheared and denatured salmon sperm DNA, and 50% formamide, following standard Southern blotting procedures for 12 to 24 h. The carrier material is finally washed three times each for 15 minutes using 2× SSC, 0.2% SDS at 70° C.

Subsequence: The term “subsequence” means a polynucleotide having one or more (e.g. several) nucleotides absent from the 5′ and/or 3′ end of a mature polypeptide coding sequence; wherein the subsequence encodes a fragment having enzymatic activity, such as activity on xanthan gum pretreated with xanthan lyase or xanthan lyase activity.

Textile: The term “textile” means any textile material including yarns, yarn intermediates, fibers, non-woven materials, natural materials, synthetic materials, and any other textile material, fabrics made of these materials and products made from fabrics (e.g. garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, ramie, cellulose acetate fibers (tricell), lyocell or blends thereof. The textile or fabric may also be non-cellulose based such as natural polyamides including wool, camel, cashmere, mohair, rabit and silk or synthetic polymer such as nylon, aramid, polyester, acrylic, polypropylen and spandex/elastane, or blends thereof as well as blend of cellulose based and non-cellulose based fibers. Examples of blends are blends of cotton and/or rayon/viscose with one or more companion material such as wool, synthetic fibers (e.g. polyamide fibers, acrylic fibers, polyester fibers, polyvinyl alcohol fibers, polyvinyl chloride fibers, polyurethane fibers, polyurea fibers, aramid fibers), and cellulose-containing fibers (e.g. rayon/viscose, ramie, flax/linen, jute, cellulose acetate fibers, lyocell). Fabric may be conventional washable laundry, for example stained household laundry. When the term fabric or garment is used it is intended to include the broader term textiles as well.

Textile care benefit: “Textile care benefits”, which are not directly related to catalytic stain removal or prevention of redeposition of soils, are also important for enzyme detergency benefits. Examples of such textile care benefits are prevention or reduction of dye transfer from one textile to another textile or another part of the same textile an effect that is also termed dye transfer inhibition or anti-backstaining, removal of protruding or broken fibers from a textile surface to decrease pilling tendencies or remove already existing pills or fuzz an effect that also is termed anti-pilling, improvement of the textile-softness, colour clarification of the textile and removal of particulate soils which are trapped in the fibers of the textile. Enzymatic bleaching is a further enzyme detergency benefit where the catalytic activity generally is used to catalyze the formation of bleaching component such as hydrogen peroxide or other peroxides or other bleaching species.

Variant: The term “variant” means a polypeptide (e.g. a GH9 endoglucanase polypeptide) comprising an alteration i.e. a substitution, insertion, and/or deletion, at one or more (e.g. several) positions. A substitution means replacement of the amino acid occupying a position with a different amino acid; a deletion means removal of the amino acid occupying a position; and an insertion means adding one or more (e.g. several) amino acids e.g. 1-5 amino acids adjacent to and immediately following the amino acid occupying a position. Non-limiting examples of endoglucanase/xanthan lyase variants described herein include endoglucanase/xanthan lyase variants having an activity on xanthan gum (for xanthan lyase) and xanthan gum pretreated with xanthan lyase (for endoglucanase). Non-limiting examples of variants escribed herein further include variants having at least 20%, e.g. at least 40%, 50%, 60%, 70%, 80%, 90%, 95%, or 100% endoglucanase activity of the mature parent having SEQ ID NO:2 or SEQ ID NO:6. A preferred assay for measuring activity on xanthan gum (optionally pretreated with xanthan lyase) is disclosed in Examples 3 and 7 herein.

Stability: The term “stability” means resistance or the degree of resistance to change, unfolding, disintegration, denaturation or activity loss. Non-limiting examples of stability include conformational stability, storage stability and stability during use, e.g. during a wash process and reflects the stability of a polypeptide (e.g. an endoglucanase or xanthan lyase variant according to the invention) as a function of time, e.g. how much activity is retained when said polypeptide (e.g. said endoglucanase or xanthan lyase variant) is kept in solution, in particular in a detergent solution. The stability is influenced by many factors, e.g. presence of chelator(s), pH, temperature, detergent composition, e.g. amount of builders, surfactants, chelators etc. The endoglucanase or xanthan lyase stability may be measured using a half-life improvement factor (HIF) as described in Examples 3 and 7 herein, e.g. relative to the parent enzyme having SEQ ID NO:2 or 6. The endoglucanase stability may also be measured using a reducing sugar assay as described in Example 3 herein.

Improved stability: The term “improved stability” or “increased stability” is defined herein as increased stability in a detergent composition (e.g. in solutions, e.g. in the presence of a chelator, e.g. EDTA or citrate), relative to the stability of the parent endoglucanase/xanthan lyase, relative to an endoglucanase/xanthan lyase having the identical amino acid sequence of the variant, but not having the alterations at one or more of the specified positions, or relative to SEQ ID NO:2 and SEQ ID NO:6, respectively. The terms “improved stability” and “increased stability” includes “improved chemical stability”, “detergent stability” and “improved detergent stability.

Improved chemical stability: The term “improved chemical stability” is defined herein as a variant enzyme displaying retention of enzymatic activity after a period of incubation in the presence of a chemical or chemicals, either naturally occurring or synthetic, which reduces the enzymatic activity of the parent enzyme. Improved chemical stability may also result in variants being more able (e.g. better that the parent) to catalyze a reaction in the presence of such chemicals. In a particular aspect of the invention the improved chemical stability is an improved stability in a detergent, in particular in a liquid detergent. The term “detergent stability” or “improved detergent stability is in particular an improved stability of the endoglucanase/xanthan lyase compared to the parent endoglucanase/xanthan lyase, when an endoglucanase variant/xanthan lyase variant of the present invention is mixed into a liquid detergent formulation, especially into a liquid detergent formulation comprising a chelator (e.g. EDTA or citrate).

Conformational stability: The term “conformational stability” means resistance or a degree of resistance to conformational change, unfolding or disintegration. Accordingly, the term “less conformationally stable” means less resistant or having lesser degree of resistance to conformational change, unfolding or disintegration.

Instability: The term “instability” means lack of stability. Non-limiting examples of instability include conformational instability, unfolding, denaturation, desintegration, activity loss.

Chelator-induced instability region: The term “chelator-induced instability region” means any region of a polypeptide contibuting to instability of said polypeptide in the presence of a chelator. Non-limiting examples of chelators include EDTA (Ethylenediaminetetraacetic acid) and citrate. Non-limiting examples of chelator-induced instability regions include any region of a polypeptide having one or more of the following features: in the presence of a chelator it is less conformationally stable than one or more or all of its adjacent regions; and/or in the presence of a chelator it is more exposed to said chelator than one or more or all of its adjacent regions; and/or in the presence of a chelator it is more accessible to said chelator than one or more or all of its adjacent regions; and/or in the presence of a chelator it is more conformationally dynamic than one or more or all of its adjacent regions; and/or in the presence of a chelator it is more receptive to deuterium incorporation than one or more or all of its adjacent regions. Non-limiting examples of chelator-induced instability regions further include any region of a polypeptide responsible for chelator-induced instability. Non-limiting examples of chelator-induced instability regions of a mature endoglucanase (e.g. having SEQ ID NO:2) or mature xanthan lyase (e.g. having SEQ ID NO:6) include the regions described above.

Adjacent region: The term “adjacent region” means any region of a polypeptide that is not a chelator-induced instability region. Non-limiting examples of adjacent regions of a mature endoglucanase (e.g. having SEQ ID NO:2) or mature xanthan lyase (e.g. having SEQ ID NO:6) hae been disclosed above.

Chelator exposure: The term “chelator exposure” means concentration or amount of a chelator that reaches a polypeptide. Accordingly, in the context of the present invention the term “more exposed to a chelator” means that chelator exposure of a particular region (e.g. a chelator-induced instability region) is greater than a chelator exposure of a different region (e.g. an adjacent region). In one aspect, chelator exposure can be expressed in numerical terms of concentration, duration, and frequency (e.g. for chemical agents, e.g. chelators) or intensity.

Chelator accessibility: The term “chelator accessibility” encompases openness to the influence by a chelator and easiness of approach by chelator. Accordingly, in the context of the present invention the term “more accessible to a chelator” means that chelator accessibility of a particular region (e.g. a chelator-induced instability region) is greater than a chelator accessibility of a different region (e.g. an adjacent region).

Conformational dynamics: The term “conformational dynamics” encompasses vibrations, structural rearrangements and transitions of a polypeptide (e.g. in solution). Accordingly, in the context of the present invention the term “more conformationally dynamic” means that conformational dynamics of a particular region (e.g. a chelator-induced instability region) is greater than conformational dynamics of a different region (e.g. an adjacent region).

Receptiveness to deuterium incorporation: The term “receptiveness to deuterium incorporation” means amount of hydrogen atoms replaced by a deuterium atoms during hydrogen-deuterium exchange. Said amount can be measured in relative (e.g. compared to another amount) or absolute (e.g. expressed numerically) terms. Accordingly, in the context of the present invention the term “more receptive to deuterium incorporation” means that receptiveness to deuterium incorporation of a particular region (e.g. a chelator-induced instability region) is greater than receptiveness to deuterium incorporation of a different region (e.g. an adjacent region).

Wash performance: The term “wash performance” is used as an enzyme's ability to remove stains present on the object to be cleaned during e.g. wash or hard surface cleaning. The improvement in the wash performance may be quantified by calculating the so-called intensity value (Int) in ‘Automatic Mechanical Stress Assay (AMSA) for laundry’ or the remission value (Rem).

Conventions for Designation of Variants

For purposes of the present invention, the mature polypeptide disclosed in SEQ ID NO:2 is used to determine the corresponding amino acid residue in another endoglucanase and the mature polypeptide disclosed in SEQ ID NO:6 is used to determine the corresponding amino acid residue in another xanthan lyase. The amino acid sequence of another endoglucanase/xanthan lyase is aligned with the mature polypeptide disclosed in SEQ ID NO:2 or SEQ ID NO:6, and based on the alignment, the amino acid position number corresponding to any amino acid residue in the mature polypeptide disclosed in SEQ ID NO:2 or 6 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another endoglucanase/xanthan lyase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537:39-64; Katoh and Toh, 2010, Bioinformatics 26:_1899-1900), and EMBOSS EMMA employing ClustalW (1.83 or later; Thompson et al., 1994, Nucleic Acids Research 22: 4673-4680), using their respective default parameters.

When the other enzyme has diverged from the mature polypeptide of SEQ ID NO:2 or 6 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example, the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g. Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g. “Gly205Arg+Ser411Phe” or “G205R+S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “Glyl95*” or “G195*”. Multiple deletions are separated by addition marks (“+”), e.g. “Glyl95*+Ser411*” or “G195*+S411*”.

Insertions. For an amino acid insertion, the following nomenclature is used: Original amino acid, position, original amino acid, inserted amino acid. Accordingly, the insertion of lysine after glycine at position 195 is designated “Glyl95GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Glyl95GlyLysAla” or “G195GKA”. An indication of an insertion ata particular position is understood as being an insertion after the original amino acid residue. For example, an “insertion at position 195” is understood to be an insertion after the original residue in position 195.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G-K-A |

Multiple alterations. Variants comprising multiple alterations are separated by addition marks (“+”), e.g. “Arg170Tyr+Glyl95Glu” or “R170Y+G195E” representing a substitution of arginine and glycine at positions 170 and 195 with tyrosine and glutamic acid, respectively.

Different alterations. Where different alterations can be introduced at a position, the different alterations are separated by a comma, e.g. “Arg170Tyr,Glu” represents a substitution of arginine at position 170 with tyrosine or glutamic acid. Thus, “Tyr167Gly,Ala+Arg170Gly,Ala” designates the following variants: “Tyr167Gly+Arg170Gly”, “Tyr167Gly+Arg170Ala”, “Tyr167Ala+Arg170Gly”, and “Tyr167Ala+Arg170Ala”. Alternatively, different alterations or may be indicated using brackets, e.g. Arg170[Tyr, Gly] or in one-letter code R170[Y,G].

### DETAILED DESCRIPTION OF THE INVENTION

The known xanthan endoglucanase having SEQ ID NO:2 and the xanthan lyase having SEQ ID NO:6 are both large enzymes (>1000 residues). It is therefore extremely laborious and expensive to target its properties for improvement of, e.g. stability in a detergent composition, e.g. in the presence of a chelator. In some aspects, the present invention narrows down the number of residues to target when trying to stabilize endoglucanase molecules using protein engineering to a region selected from the group consisting of: region 1 corresponding to amino acids 95 to 105 of SEQ ID NO:2, region 2 corresponding to amino acids 115 to 138 of SEQ ID NO:2, region 3 corresponding to amino acids 210 to 251 of SEQ ID NO:2, region 4 corresponding to amino acids 267 to 301 of SEQ ID NO:2, region 5 corresponding to amino acids 339 to 361 of SEQ ID NO:2, region 6 corresponding to amino acids 547 to 595 of SEQ ID NO:2, region 7 corresponding to amino acids 612 to 660 of SEQ ID NO:2, region 8 corresponding to amino acids 806 to 828 of SEQ ID NO:2, and region 9 corresponding to amino acids 839 to 1042 of SEQ ID NO:2, and/or region 10 corresponding to amino acids 1 to 94 of SEQ ID NO:2, region 11 corresponding to amino acids 106 to 114 of SEQ ID NO:2, region 12 corresponding to amino acids 139 to 209 of SEQ ID NO:2, region 13 corresponding to amino acids 252 to 266 of SEQ ID NO:2, region 14 corresponding to amino acids 302 to 338 of SEQ ID NO:2, region 15 corresponding to amino acids 362 to 546 of SEQ ID NO:2, region 16 corresponding to amino acids 596 to 611 of SEQ ID NO:2, region 17 corresponding to amino acids 661 to 805 of SEQ ID NO:2, region 18 corresponding to amino acids 829 to 838 of SEQ ID NO:2, and region 19 corresponding to amino acids 1043 to 1055 of SEQ ID NO:2.

In some aspects, the present invention narrows down the number of residues to target when trying to stabilize xanthan lyase molecules using protein engineering to a region selected from the group consisting of: region 1 corresponding to amino acids 154 to 176 of SEQ ID NO:6, region 2 corresponding to amino acids 614 to 658 of SEQ ID NO:6, region 3 corresponding to amino acids 731 to 803 of SEQ ID NO:6, region 4 corresponding to amino acids 807 to 846 of SEQ ID NO:6, region 5 corresponding to amino acids 872 to 885 of SEQ ID NO:6, and region 6 corresponding to amino acids 903 to 1004 of SEQ ID NO:6, and/or region 7 corresponding to amino acids 1 to 153 of SEQ ID NO:6, region 8 corresponding to amino acids 177 to 613 of SEQ ID NO:6, region 9 corresponding to amino acids 659 to 730 of SEQ ID NO:6, region 10 corresponding to amino acids 804 to 806 of SEQ ID NO:6, region 11 corresponding to amino acids 847 to 871 of SEQ ID NO:6, region 12 corresponding to amino acids 886 to 902 of SEQ ID NO:6, and region 13 corresponding to amino acids 1005 to 1037 of SEQ ID NO:6.

In one embodiment, the present invention dramatically narrows down the number of residues to target when trying to stabilize endoglucanase/xanthan lyase molecules using protein engineering.

In another embodiment, the present invention provides detergent compositions comprising variants of an endoglucanase and of a xanthan lyase, as described herein, both of which have significantly improved stability as compared to the parent enzyme, such as the wild-type endoglucanase/xanthan lyase. Such improved stability may be measured as improved half-life of the variant compared to the parent endoglucanase/xanthan lyase, such as a wild-type endoglucanase/xanthan lyase. Furthermore, the stability of the variant is also proven to be improved in the presence of a protease, which normally would cleave proteins. The present invention discloses variants that have been modified so that they have an improved stability towards protease cleavage.

Variants

In one embodiment, chelator-induced instability regions in the protein sequence of the known xanthan endoglucanase having SEQ ID NO:2 that are affected when the molecule is incubated in a buffer with EDTA, are the following: region 1 corresponding to amino acids 95 to 105 of SEQ ID NO:2, region 2 corresponding to amino acids 115 to 138 of SEQ ID NO:2, region 3 corresponding to amino acids 210 to 251 of SEQ ID NO:2, region 4 corresponding to amino acids 267 to 301 of SEQ ID NO:2, region 5 corresponding to amino acids 339 to 361 of SEQ ID NO:2, region 6 corresponding to amino acids 547 to 595 of SEQ ID NO:2, region 7 corresponding to amino acids 612 to 660 of SEQ ID NO:2, region 8 corresponding to amino acids 806 to 828 of SEQ ID NO:2, and region 9 corresponding to amino acids 839 to 1042 of SEQ ID NO:2. This embodiment relates to an important guidance on where to mutate an endoglucanase in order to stabilize its molecule in a detergent, e.g. detergent composition comprising a chelator, e.g. EDTA or citrate.

Accordingly, in one embodiment the present invention relates to a detergent composition comprising an endoglucanase variant, comprising an alteration (e.g. a substitution, deletion or insertion, preferably a substitution) at one or more positions in a region selected from the group consisting of: region 1 corresponding to amino acids 95 to 105 of SEQ ID NO:2, region 2 corresponding to amino acids 115 to 138 of SEQ ID NO:2, region 3 corresponding to amino acids 210 to 251 of SEQ ID NO:2, region 4 corresponding to amino acids 267 to 301 of SEQ ID NO:2, region 5 corresponding to amino acids 339 to 361 of SEQ ID NO:2, region 6 corresponding to amino acids 547 to 595 of SEQ ID NO:2, region 7 corresponding to amino acids 612 to 660 of SEQ ID NO:2, region 8 corresponding to amino acids 806 to 828 of SEQ ID NO:2, and region 9 corresponding to amino acids 839 to 1042 of SEQ ID NO:2, wherein said variant has at least 60% and less than 100% sequence identity to SEQ ID NO:2; preferably said endoglucanase variant has activity on xanthan gum pretreated with xanthan lyase, further preferably said activity is a xanthan gum degrading activity.

In one embodiment, regions in the protein sequence of the known xanthan endoglucanase having SEQ ID NO:2 that are affected when the molecule is incubated in a detergent, are the following: region 10 corresponding to amino acids 1 to 94 of SEQ ID NO:2, region 11 corresponding to amino acids 106 to 114 of SEQ ID NO:2, region 12 corresponding to amino acids 139 to 209 of SEQ ID NO:2, region 13 corresponding to amino acids 252 to 266 of SEQ ID NO:2, region 14 corresponding to amino acids 302 to 338 of SEQ ID NO:2, region 15 corresponding to amino acids 362 to 546 of SEQ ID NO:2, region 16 corresponding to amino acids 596 to 611 of SEQ ID NO:2, region 17 corresponding to amino acids 661 to 805 of SEQ ID NO:2, region 18 corresponding to amino acids 829 to 838 of SEQ ID NO:2, and region 19 corresponding to amino acids 1043 to 1055 of SEQ ID NO:2. This embodiment relates to an important guidance on where to mutate an endoglucanase in order to stabilize its molecule in a detergent.

In one embodiment the present invention relates to a detergent composition comprising an endoglucanase variant, comprising comprising an alteration (e.g. a substitution, deletion or insertion, preferably a substitution) at one or more positions in a region selected from the group consisting of: region 10 corresponding to amino acids 1 to 94 of SEQ ID NO:2, region 11 corresponding to amino acids 106 to 114 of SEQ ID NO:2, region 12 corresponding to amino acids 139 to 209 of SEQ ID NO:2, region 13 corresponding to amino acids 252 to 266 of SEQ ID NO:2, region 14 corresponding to amino acids 302 to 338 of SEQ ID NO:2, region 15 corresponding to amino acids 362 to 546 of SEQ ID NO:2, region 16 corresponding to amino acids 596 to 611 of SEQ ID NO:2, region 17 corresponding to amino acids 661 to 805 of SEQ ID NO:2, region 18 corresponding to amino acids 829 to 838 of SEQ ID NO:2, and region 19 corresponding to amino acids 1043 to 1055 of SEQ ID NO:2; preferably said endoglucanase variant has activity on xanthan gum pretreated with xanthan lyase, further preferably said activity is a xanthan gum degrading activity.

In one embodiment the present invention relates to a detergent composition comprising an endoglucanase variant, comprising an alteration (e.g. a substitution, deletion or insertion, preferably a substitution) at one or more positions in a region selected from the group consisting of:

i) region 1 corresponding to amino acids 95 to 105 of SEQ ID NO:2, e.g. said alteration at one or more positions selected from the group consisting of positions: 95, 96, 97, 98, 99, 100, 101, 102, 103, 104, and 105, wherein said positions correspond to amino acid positions of SEQ ID NO:2 (e.g. using the numbering of SEQ ID NO:2),

ii) region 2 corresponding to amino acids 115 to 138 of SEQ ID NO:2, e.g. said alteration at one or more positions selected from the group consisting of positions: 115, 116, 117, 118, 119, 120, 121, 122, 123, 124, 125, 126, 127, 128, 129, 130, 131, 132, 133, 134, 135, 136, 137, and 138, wherein said positions correspond to amino acid positions of SEQ ID NO:2 (e.g. using the numbering of SEQ ID NO:2),

iii) region 3 corresponding to amino acids 210 to 251 of SEQ ID NO:2, e.g. said alteration at one or more positions selected from the group consisting of positions: 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, and 251, wherein said positions correspond to amino acid positions of SEQ ID NO:2 (e.g. using the numbering of SEQ ID NO:2),

iv) region 4 corresponding to amino acids 267 to 301 of SEQ ID NO:2, e.g. said alteration at one or more positions selected from the group consisting of positions: 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, and 301, wherein said positions correspond to amino acid positions of SEQ ID NO:2 (e.g. using the numbering of SEQ ID NO:2),

v) region 5 corresponding to amino acids 339 to 361 of SEQ ID NO:2, e.g. said alteration at one or more positions selected from the group consisting of positions: 339, 340, 341, 342, 343, 344, 345, 346, 347, 348, 349, 350, 351, 352, 353, 354, 355, 356, 357, 358, 359, 360, 361, wherein said positions correspond to amino acid positions of SEQ ID NO:2 (e.g. using the numbering of SEQ ID NO:2),

vi) region 6 corresponding to amino acids 547 to 595 of SEQ ID NO:2, e.g. said alteration at one or more positions selected from the group consisting of positions: 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, and 595, wherein said positions correspond to amino acid positions of SEQ ID NO:2 (e.g. using the numbering of SEQ ID NO:2),

vii) region 7 corresponding to amino acids 612 to 660 of SEQ ID NO:2, e.g. said alteration at one or more positions selected from the group consisting of positions: 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, and 660, wherein said positions correspond to amino acid positions of SEQ ID NO:2 (e.g. using the numbering of SEQ ID NO:2),

viii) region 8 corresponding to amino acids 806 to 828 of SEQ ID NO:2, e.g. said alteration at one or more positions selected from the group consisting of positions: 806, 807, 808, 809, 810, 811, 812, 813, 814, 815, 816, 817, 818, 819, 820, 821, 822, 823, 824, 825, 826, 827, and 828, wherein said positions correspond to amino acid positions of SEQ ID NO:2 (e.g. using the numbering of SEQ ID NO:2), and

ix) region 9 corresponding to amino acids 839 to 1042 of SEQ ID NO:2, e.g. said alteration at one or more positions selected from the group consisting of positions: 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, and 1042, wherein said positions correspond to amino acid positions of SEQ ID NO:2 (e.g. using the numbering of SEQ ID NO:2).

In one embodiment, the present invention relates to a detergent composition comprising an endoglucanase variant, comprising an alteration (e.g. a substitution, deletion or insertion, preferably a substitution) at one or more positions in a region selected from the group consisting of:

i) region 10 corresponding to amino acids 1 to 94 of SEQ ID NO:2, e.g. said alteration at one or more positions selected from the group consisting of positions: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, wherein said positions correspond to amino acid positions of SEQ ID NO:2 (e.g. using the numbering of SEQ ID NO:2),

ii) region 11 corresponding to amino acids 106 to 114 of SEQ ID NO:2, e.g. said alteration at one or more positions selected from the group consisting of positions: 107, 108, 109, 110, 111, 112, 113, 114, wherein said positions correspond to amino acid positions of SEQ ID NO:2 (e.g. using the numbering of SEQ ID NO:2),

iii) region 12 corresponding to amino acids 139 to 209of SEQ ID NO:2, e.g. said alteration at one or more positions selected from the group consisting of positions: 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, wherein said positions correspond to amino acid positions of SEQ ID NO:2 (e.g. using the numbering of SEQ ID NO:2),

iv) region 13 corresponding to amino acids 252 to 266 of SEQ ID NO:2, e.g. said alteration at one or more positions selected from the group consisting of positions: 252, 253, 254, 255, 256, 257, 258, 259, 260, 261, 262, 263, 264, 265, 266, wherein said positions correspond to amino acid positions of SEQ ID NO:2 (e.g. using the numbering of SEQ ID NO:2),

v) region 14 corresponding to amino acids 302 to 338 of SEQ ID NO:2, e.g. said alteration at one or more positions selected from the group consisting of positions: 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, wherein said positions correspond to amino acid positions of SEQ ID NO:2 (e.g. using the numbering of SEQ ID NO:2),

vi) region 15 corresponding to amino acids 362 to 546 of SEQ ID NO:2, e.g. said alteration at one or more positions selected from the group consisting of positions: 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, wherein said positions correspond to amino acid positions of SEQ ID NO:2 (e.g. using the numbering of SEQ ID NO:2),

vii) region 16 corresponding to amino acids 596 to 611 of SEQ ID NO:2, e.g. said alteration at one or more positions selected from the group consisting of positions: 596, 597, 598, 599, 600, 601, 602, 603, 604, 605, 606, 607, 608, 609, 610, 611, wherein said positions correspond to amino acid positions of SEQ ID NO:2 (e.g. using the numbering of SEQ ID NO:2),

viii) region 17 corresponding to amino acids 661 to 805 of SEQ ID NO:2, e.g. said alteration at one or more positions selected from the group consisting of positions: 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, , 781, 782, 783, 784, 785, 786, 787, 788, 789, 790, 791, 792, 793, 794, 795, 796, 797, 798, 799, 800, 801, 802, 803, 804, 805, wherein said positions correspond to amino acid positions of SEQ ID NO:2 (e.g. using the numbering of SEQ ID NO:2),

ix) region 18 corresponding to amino acids 829 to 838 of SEQ ID NO:2, e.g. said alteration at one or more positions selected from the group consisting of positions: 829, 830, 831, 832, 833, 834, 835, 836, 837, 838, wherein said positions correspond to amino acid positions of SEQ ID NO:2 (e.g. using the numbering of SEQ ID NO:2) x) region 19 corresponding to amino acids 1043 to 1055 of SEQ ID NO:2, e.g. said alteration at one or more positions selected from the group consisting of positions 1043, 1044, 1045, 1046, 1047, 1048, 1049, 1050, 1051, 1052, 1053, 1054, 1055, wherein said positions correspond to amino acid positions of SEQ ID NO:2 (e.g. using the numbering of SEQ ID NO:2).

In one embodiment the present invention relates to a detergent composition comprising an endoglucanase variant, comprising an alteration (e.g. a substitution, deletion or insertion, preferably a substitution) at one or more positions in one or more regions or multiple alterations (such as 2, 3, 4, 5, 6, 7, 8, 9 or 10) in one region or multiple alterations (e.g. 2, 3, 4, 5, 6, 7, 8, 9 or 10) in multiple regions (e.g. 2, 3, 4, 5, 6, 7, 8, or 9) selected from the group consisting of: region 1 corresponding to amino acids 95 to 105 of SEQ ID NO:2, region 2 corresponding to amino acids 115 to 138 of SEQ ID NO:2, region 3 corresponding to amino acids 210 to 251 of SEQ ID NO:2, region 4 corresponding to amino acids 267 to 301 of SEQ ID NO:2, region 5 corresponding to amino acids 339 to 361 of SEQ ID NO:2, region 6 corresponding to amino acids 547 to 595 of SEQ ID NO:2, region 7 corresponding to amino acids 612 to 660 of SEQ ID NO:2, region 8 corresponding to amino acids 806 to 828 of SEQ ID NO:2, and region 9 corresponding to amino acids 839 to 1042 of SEQ ID NO:2, wherein said variant has at least 60% and less than 100% sequence identity to SEQ ID NO:2; preferably said endoglucanase variant has activity on xanthan gum pretreated with xanthan lyase, further preferably said activity is a xanthan gum degrading activity.

In one embodiment the present invention relates to a detergent composition comprising an endoglucanase variant, comprising an alteration (e.g. a substitution, deletion or insertion, preferably a substitution) at one or more positions in one or more regions selected from the group consisting of: region 10 corresponding to amino acids 1 to 94 of SEQ ID NO:2, region 11 corresponding to amino acids 106 to 114 of SEQ ID NO:2, region 12 corresponding to amino acids 139 to 209 of SEQ ID NO:2, region 13 corresponding to amino acids 252 to 266 of SEQ ID NO:2, region 14 corresponding to amino acids 302 to 338 of SEQ ID NO:2, region 15 corresponding to amino acids 362 to 546 of SEQ ID NO:2, region 16 corresponding to amino acids 596 to 611 of SEQ ID NO:2, region 17 corresponding to amino acids 661 to 805 of SEQ ID NO:2, region 18 corresponding to amino acids 829 to 838 of SEQ ID NO:2, and region 19 corresponding to amino acids 1043 to 1055 of SEQ ID NO:2, wherein said variant has at least 60% and less than 100% sequence identity to SEQ ID NO:2; preferably said endoglucanase variant has activity on xanthan gum pretreated with xanthan lyase, further preferably said activity is a xanthan gum degrading activity.

In one embodiment the present invention relates to a detergent composition comprising an endoglucanase variant as described herein having multiple alterations (such as 2, 3, 4, 5, 6, 7, 8, 9 or 10) in one region (e.g. of SEQ ID NO:2 or another parent endoglucanase) selected from the group consisting of: region 10 corresponding to amino acids 1 to 94 of SEQ ID NO:2, region 11 corresponding to amino acids 106 to 114 of SEQ ID NO:2, region 12 corresponding to amino acids 139 to 209 of SEQ ID NO:2, region 13 corresponding to amino acids 252 to 266 of SEQ ID NO:2, region 14 corresponding to amino acids 302 to 338 of SEQ ID NO:2, region 15 corresponding to amino acids 362 to 546 of SEQ ID NO:2, region 16 corresponding to amino acids 596 to 611 of SEQ ID NO:2, region 17 corresponding to amino acids 661 to 805 of SEQ ID NO:2, region 18 corresponding to amino acids 829 to 838 of SEQ ID NO:2, and region 19 corresponding to amino acids 1043 to 1055 of SEQ ID NO:2, wherein said variant has at least 60% and less than 100% sequence identity to SEQ ID NO:2, preferably said endoglucanase variant has activity on xanthan gum pretreated with xanthan lyase, further preferably said activity is a xanthan gum degrading activity.

In one embodiment the present invention relates to a detergent composition comprising an endoglucanase variant as described herein having multiple alterations (e.g. 2, 3, 4, 5, 6, 7, 8, 9 or 10) in multiple regions (e.g. 2, 3, 4, 5, 6, 7, 8, 9 or 10) (e.g. of SEQ ID NO:2 or another parent endoglucanase) selected from the group consisting of: region 10 corresponding to amino acids 1 to 94 of SEQ ID NO:2, region 11 corresponding to amino acids 106 to 114 of SEQ ID NO:2, region 12 corresponding to amino acids 139 to 209 of SEQ ID NO:2, region 13 corresponding to amino acids 252 to 266 of SEQ ID NO:2, region 14 corresponding to amino acids 302 to 338 of SEQ ID NO:2, region 15 corresponding to amino acids 362 to 546 of SEQ ID NO:2, region 16 corresponding to amino acids 596 to 611 of SEQ ID NO:2, region 17 corresponding to amino acids 661 to 805 of SEQ ID NO:2, region 18 corresponding to amino acids 829 to 838 of SEQ ID NO:2, and region 19 corresponding to amino acids 1043 to 1055 of SEQ ID NO:2, preferably said endoglucanase variant has activity on xanthan gum pretreated with xanthan lyase, further preferably said activity is a xanthan gum degrading activity. In one embodiment the present invention relates to a detergent composition comprising a parent endoglucanase of the invention (e.g. SEQ ID NO:2) having an alteration (e.g. a substitution, deletion or insertion, preferably a substitution) at one or more positions in a region selected from the group consisting of regions 1-9, wherein said region is a chelator-induced instability region, preferably said chelator-induced instability region has one or more of the following features: in the presence of a chelator it is less conformationally stable than one or more or all of its adjacent regions; and/or in the presence of a chelator it is more exposed to said chelator than one or more or all of its adjacent regions; and/or in the presence of a chelator it is more accessible to said chelator than one or more or all of its adjacent regions; and/or in the presence of a chelator it is more conformationally dynamic than one or more or all of its adjacent regions; and/or in the presence of a chelator it is more receptive to deuterium incorporation than one or more or all of its adjacent regions; further preferably said adjacent region is selected from the group consisting of: region 10 corresponding to amino acids 1 to 94 of SEQ ID NO:2, region 11 corresponding to amino acids 106 to 114 of SEQ ID NO:2, region 12 corresponding to amino acids 139 to 209 of SEQ ID NO:2, region 13 corresponding to amino acids 252 to 266 of SEQ ID NO:2, region 14 corresponding to amino acids 302 to 338 of SEQ ID NO:2, region 15 corresponding to amino acids 362 to 546 of SEQ ID NO:2, region 16 corresponding to amino acids 596 to 611 of SEQ ID NO:2, region 17 corresponding to amino acids 661 to 805 of SEQ ID NO:2, region 18 corresponding to amino acids 829 to 838 of SEQ ID NO:2, and region 19 corresponding to amino acids 1043 to 1055 of SEQ ID NO:2; further most preferably said chelator is EDTA or citrate.

In one embodiment the adjacent regions referred to herein can be one or more or all of the following: region 10 corresponding to amino acids 1 to 94 of SEQ ID NO:2, region 11 corresponding to amino acids 106 to 114 of SEQ ID NO:2, region 12 corresponding to amino acids 139 to 209 of SEQ ID NO:2, region 13 corresponding to amino acids 252 to 266 of SEQ ID NO:2, region 14 corresponding to amino acids 302 to 338 of SEQ ID NO:2, region 15 corresponding to amino acids 362 to 546 of SEQ ID NO:2, region 16 corresponding to amino acids 596 to 611 of SEQ ID NO:2, region 17 corresponding to amino acids 661 to 805 of SEQ ID NO:2, region 18 corresponding to amino acids 829 to 838 of SEQ ID NO:2, and region 19 corresponding to amino acids 1043 to 1055 of SEQ ID NO:2.

In one embodiment the present invention relates to a detergent composition comprising an endoglucanase variant of the invention having an alteration (e.g. a substitution, deletion or insertion, preferably a substitution) at one or more positions in a region selected from the group consisting of regions 1-9 (e.g. of SEQ ID NO:2 or another parent endoglucanase), wherein in an aqueous solution comprising a detergent component said region (e.g. of SEQ ID NO:2 or another parent endoglucanase) is relatively more accessible to said detergent component than one or more or all of its adjacent regions.

In one embodiment the present invention relates to a detergent composition comprising an endoglucanase variant of the invention having an alteration (e.g. a substitution, deletion or insertion, preferably a substitution) at one or more positions in a region selected from the group consisting of regions 1-9 (e.g. of SEQ ID NO:2 or another parent endoglucanase), wherein in an aqueous solution comprising a detergent component said region (e.g. of SEQ ID NO:2 or another parent endoglucanase) is relatively more exposed to said detergent component than one or more or all of its adjacent regions.

In one embodiment the present invention relates to a detergent composition comprising an endoglucanase variant of the invention having an alteration (e.g. a substitution, deletion or insertion, preferably a substitution) at one or more positions in a region selected from the group consisting of regions 1-9 (e.g. of SEQ ID NO:2 or another parent endoglucanase), wherein in an aqueous solution comprising a detergent component said region (e.g. of SEQ ID NO:2 or another parent endoglucanase) is relatively more accessible to said detergent component than one or more or all of its adjacent regions.

In one embodiment the present invention relates to a detergent composition comprising an endoglucanase variant of the invention having an alteration (e.g. a substitution, deletion or insertion, preferably a substitution) at one or more positions in a region selected from the group consisting of regions 1-9 (e.g. of SEQ ID NO:2 or another parent endoglucanase), wherein in an aqueous solution comprising a detergent component said region (e.g. of SEQ ID NO:2 or another parent endoglucanase) is relatively more conformationally dynamic than one or more or all of its adjacent regions.

In one embodiment the present invention relates to a detergent composition comprising an endoglucanase variant of the invention having an alteration (e.g. a substitution, deletion or insertion, preferably a substitution) at one or more positions in a region selected from the group consisting of regions 1-9 (e.g. of SEQ ID NO:2 or another parent endoglucanase), wherein in an aqueous solution comprising a detergent component said region (e.g. of SEQ ID NO:2 or another parent endoglucanase) is relatively more receptive to deuterium incorporation than one or more or all of its adjacent regions.

In one embodiment the present invention relates to a detergent composition comprising an endoglucanase variant of the invention, further comprising an alteration (e.g. a substitution, deletion or insertion, preferably a substitution) at one or more positions in one or more regions selected from the group consisting of: region 1 corresponding to amino acids 95 to 105 of SEQ ID NO:2, region 2 corresponding to amino acids 115 to 138 of SEQ ID NO:2, region 3 corresponding to amino acids 210 to 251 of SEQ ID NO:2, region 4 corresponding to amino acids 267 to 301 of SEQ ID NO:2, region 5 corresponding to amino acids 339 to 361 of SEQ ID NO:2, region 6 corresponding to amino acids 547 to 595 of SEQ ID NO:2, region 7 corresponding to amino acids 612 to 660 of SEQ ID NO:2, region 8 corresponding to amino acids 806 to 828 of SEQ ID NO:2, and region 9 corresponding to amino acids 839 to 1042 of SEQ ID NO:2, wherein said variant has at least 60% and less than 100% sequence identity to SEQ ID NO:2, preferably said endoglucanase variant has activity on xanthan gum pretreated with xanthan lyase, further preferably said activity is a xanthan gum degrading activity.

In one embodiment the present invention relates to a detergent composition comprising an endoglucanase variant of the invention having multiple alterations (such as 2, 3, 4, 5, 6, 7, 8, 9 or 10) in one region (e.g. of SEQ ID NO:2 or another parent endoglucanase) selected from the group consisting of: region 1 corresponding to amino acids 95 to 105 of SEQ ID NO:2, region 2 corresponding to amino acids 115 to 138 of SEQ ID NO:2, region 3 corresponding to amino acids 210 to 251 of SEQ ID NO:2, region 4 corresponding to amino acids 267 to 301 of SEQ ID NO:2, region 5 corresponding to amino acids 339 to 361 of SEQ ID NO:2, region 6 corresponding to amino acids 547 to 595 of SEQ ID NO:2, region 7 corresponding to amino acids 612 to 660 of SEQ ID NO:2, region 8 corresponding to amino acids 806 to 828 of SEQ ID NO:2, and region 9 corresponding to amino acids 839 to 1042 of SEQ ID NO:2, wherein said variant has at least 60% and less than 100% sequence identity to SEQ ID NO:2, preferably said endoglucanase variant has activity on xanthan gum pretreated with xanthan lyase, further preferably said activity is a xanthan gum degrading activity.

In one embodiment the present invention relates to a detergent composition comprising an endoglucanase variant of the invention having multiple alterations (e.g. 2, 3, 4, 5, 6, 7, 8, 9 or 10) in multiple regions (e.g. 2, 3, 4, 5, 6, 7, 8, or 9) (e.g. of SEQ ID NO:2 or another parent endoglucanase) selected from the group consisting of: region 1 corresponding to amino acids 95 to 105 of SEQ ID NO:2, region 2 corresponding to amino acids 115 to 138 of SEQ ID NO:2, region 3 corresponding to amino acids 210 to 251 of SEQ ID NO:2, region 4 corresponding to amino acids 267 to 301 of SEQ ID NO:2, region 5 corresponding to amino acids 339 to 361 of SEQ ID NO:2, region 6 corresponding to amino acids 547 to 595 of SEQ ID NO:2, region 7 corresponding to amino acids 612 to 660 of SEQ ID NO:2, region 8 corresponding to amino acids 806 to 828 of SEQ ID NO:2, and region 9 corresponding to amino acids 839 to 1042 of SEQ ID NO:2, wherein said variant has at least 60% and less than 100% sequence identity to SEQ ID NO:2, preferably said endoglucanase variant has activity on xanthan gum pretreated with xanthan lyase, further preferably said activity is a xanthan gum degrading activity.

In a further embodiment, the present invention relates to a detergent composition comprising an endoglucanase variant of the invention having multiple alterations (e.g. 2, 3, 4, 5, 6, 7, 8, 9 or 10) in multiple regions (e.g. 2, 3, 4, 5, 6, 7, 8, or 9) (e.g. of SEQ ID NO:2 or another parent endoglucanase) selected from the group consisting of: region 1 corresponding to amino acids 95 to 105 of SEQ ID NO:2, region 2 corresponding to amino acids 115 to 138 of SEQ ID NO:2, region 3 corresponding to amino acids 210 to 251 of SEQ ID NO:2, region 4 corresponding to amino acids 267 to 301 of SEQ ID NO:2, region 5 corresponding to amino acids 339 to 361 of SEQ ID NO:2, region 6 corresponding to amino acids 547 to 595 of SEQ ID NO:2, region 7 corresponding to amino acids 612 to 660 of SEQ ID NO:2, region 8 corresponding to amino acids 806 to 828 of SEQ ID NO:2, and region 9 corresponding to amino acids 839 to 1042 of SEQ ID NO:2, and multiple alterations (e.g. 2, 3, 4, 5, 6, 7, 8, 9, or 10) in multiple adjacent regions (e.g. 2, 3, 4, 5, 6, 7, 8, 9, or 10) selected from the group consisting of: region 10 corresponding to amino acids 1 to 94 of SEQ ID NO:2, region 11 corresponding to amino acids 106 to 114 of SEQ ID NO:2, region 12 corresponding to amino acids 139 to 209 of SEQ ID NO:2, region 13 corresponding to amino acids 252 to 266 of SEQ ID NO:2, region 14 corresponding to amino acids 302 to 338 of SEQ ID NO:2, region 15 corresponding to amino acids 362 to 546 of SEQ ID NO:2, region 16 corresponding to amino acids 596 to 611 of SEQ ID NO:2, region 17 corresponding to amino acids 661 to 805 of SEQ ID NO:2, region 18 corresponding to amino acids 829 to 838 of SEQ ID NO:2, and region 19 corresponding to amino acids 1043 to 1055 of SEQ ID NO:2; wherein said variant has at least 60% and less than 100% sequence identity to SEQ ID NO:2, preferably said endoglucanase variant has activity on xanthan gum pretreated with xanthan lyase, further preferably said activity is a xanthan gum degrading activity.

In one embodiment, the present invention relates to a detergent composition comprising endoglucanase variants, comprising an alteration (e.g. a substitution, deletion or insertion, preferably a substitution) at one or more (e.g. several) positions of the mature parent polypeptide (e.g. SEQ ID NO:2), wherein each alteration is independently a substitution, insertion or deletion, wherein the variant has endoglucanase activity.

In an embodiment, the variant has sequence identity of at least 60%, e.g. at least 65%, 70%, 75%, 80%, 85%, 90%, 91%, 92%, 93%, 94%, 95%, 96%, 97%, 98%, or 99%, but less than 100%, to the amino acid sequence of the parent endoglucanase.

In one embodiment, the variant has at least 60%, e.g. at least 65%, 70%, 75%, 80%, 85%, 90%, 91%, 92%, 93%, 94%, 95%, 96%, 97%, 98%, or 99%, but less than 100%, sequence identity to the mature polypeptide of SEQ ID NO:2.

In one embodiment the present invention relates to a detergent composition comprising an endoglucanase variant of the invention, wherein said variant has at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:2.

In another aspect, a variant comprises an alteration at one or more (e.g. several) positions corresponding to positions 4, 17, 18, 20, 51, 53, 55, 56, 60, 63, 71, 79, 87, 92, 99, 120, 125, 126, 130, 137, 182, 186, 189, 192, 213, 216, 221, 226, 228, 230, 231, 232, 233, 235, 240, 243, 247, 249, 278, 279, 281, 283, 285, 289, 292, 294, 298, 302, 311, 313, 333, 346, 353, 358, 386, 387, 388, 390, 403, 408, 410, 416, 441, 448, 451, 471, 472, 476, 489, 507, 512, 515, 538, 555, 556, 557, 558, 559, 560, 561, 562, 563, 564, 567, 568, 570, 575, 578, 579, 580, 581, 583, 589, 590, 591, 592, 593, 595, 598, 599, 602, 603, 605, 607, 609, 616, 627, 630, 631, 635, 636, 638, 639, 640, 641, 642, 643, 644, 651, 676, 683, 688, 690, 694, 698, 699, 706, 711, 713, 1719, 720, 744, 749, 754, 756, 760, , 781, 786, 797, 810, 811, 812, 815, 823, 824, 825, 827, 828, 833, 834, 835, 837, 843, 848, 868, 869, 870, 871, 872, 873, 874, 880, 881, 883, 884, 885, 887, 888, 890, 892, 894, 898, 905, 906, 912, 920, 921, 924, 926, 927, 928, 932, 933, 934, 935, 937, 938, 939, 940, 941, 942, 943, 946, 948, 950, 952, 953, 954, 956, 957, 960, 966, 971, 972, 980, 989, 991, 994, 995, 998, 999, 1006, 1009, 1010, 1011, 1029, 1030, 1031, 1032, 1035, 1037, 1038, 1040, 1041, 1042, 1044, 1045, 1048, wherein numbering is according to SEQ ID NO:2.

In another aspect, a variant comprises an alteration at two positions corresponding to any of positions 4, 17, 18, 20, 51, 53, 55, 56, 60, 63, 71, 79, 87, 92, 99, 120, 125, 126, 130, 137, 182, 186, 189, 192, 213, 216, 221, 226, 228, 230, 231, 232, 233, 235, 240, 243, 247, 249, 278, 279, 281, 283, 285, 289, 292, 294, 298, 302, 311, 313, 333, 346, 353, 358, 386, 387, 388, 390, 403, 408, 410, 416, 441, 448, 451, 471, 472, 476, 489, 507, 512, 515, 538, 555, 556, 557, 558, 559, 560, 561, 562, 563, 564, 567, 568, 570, 575, 578, 579, 580, 581, 583, 589, 590, 591, 592, 593, 595, 598, 599, 602, 603, 605, 607, 609, 616, 627, 630, 631, 635, 636, 638, 639, 640, 641, 642, 643, 644, 651, 676, 683, 688, 690, 694, 698, 699, 706, 711, 713, 1719, 720, 744, 749, 754, 756, 760, , 781, 786, 797, 810, 811, 812, 815, 823, 824, 825, 827, 828, 833, 834, 835, 837, 843, 848, 868, 869, 870, 871, 872, 873, 874, 880, 881, 883, 884, 885, 887, 888, 890, 892, 894, 898, 905, 906, 912, 920, 921, 924, 926, 927, 928, 932, 933, 934, 935, 937, 938, 939, 940, 941, 942, 943, 946, 948, 950, 952, 953, 954, 956, 957, 960, 966, 971, 972, 980, 989, 991, 994, 995, 998, 999, 1006, 1009, 1010, 1011, 1029, 1030, 1031, 1032, 1035, 1037, 1038, 1040, 1041, 1042, 1044, 1045, 1048, wherein numbering is according to SEQ ID NO:2.

In another aspect, a variant comprises an alteration at three positions corresponding to any of positions 4, 17, 18, 20, 51, 53, 55, 56, 60, 63, 71, 79, 87, 92, 99, 120, 125, 126, 130, 137, 182, 186, 189, 192, 213, 216, 221, 226, 228, 230, 231, 232, 233, 235, 240, 243, 247, 249, 278, 279, 281, 283, 285, 289, 292, 294, 298, 302, 311, 313, 333, 346, 353, 358, 386, 387, 388, 390, 403, 408, 410, 416, 441, 448, 451, 471, 472, 476, 489, 507, 512, 515, 538, 555, 556, 557, 558, 559, 560, 561, 562, 563, 564, 567, 568, 570, 575, 578, 579, 580, 581, 583, 589, 590, 591, 592, 593, 595, 598, 599, 602, 603, 605, 607, 609, 616, 627, 630, 631, 635, 636, 638, 639, 640, 641, 642, 643, 644, 651, 676, 683, 688, 690, 694, 698, 699, 706, 711, 713, 1719, 720, 744, 749, 754, 756, 760, , 781, 786, 797, 810, 811, 812, 815, 823, 824, 825, 827, 828, 833, 834, 835, 837, 843, 848, 868, 869, 870, 871, 872, 873, 874, 880, 881, 883, 884, 885, 887, 888, 890, 892, 894, 898, 905, 906, 912, 920, 921, 924, 926, 927, 928, 932, 933, 934, 935, 937, 938, 939, 940, 941, 942, 943, 946, 948, 950, 952, 953, 954, 956, 957, 960, 966, 971, 972, 980, 989, 991, 994, 995, 998, 999, 1006, 1009, 1010, 1011, 1029, 1030, 1031, 1032, 1035, 1037, 1038, 1040, 1041, 1042, 1044, 1045, 1048, wherein numbering is according to SEQ ID NO:2.

In another aspect, a variant comprises an alteration at each position (or at least four positions) corresponding to positions 4, 17, 18, 20, 51, 53, 55, 56, 60, 63, 71, 79, 87, 92, 99, 120, 125, 126, 130, 137, 182, 186, 189, 192, 213, 216, 221, 226, 228, 230, 231, 232, 233, 235, 240, 243, 247, 249, 278, 279, 281, 283, 285, 289, 292, 294, 298, 302, 311, 313, 333, 346, 353, 358, 386, 387, 388, 390, 403, 408, 410, 416, 441, 448, 451, 471, 472, 476, 489, 507, 512, 515, 538, 555, 556, 557, 558, 559, 560, 561, 562, 563, 564, 567, 568, 570, 575, 578, 579, 580, 581, 583, 589, 590, 591, 592, 593, 595, 598, 599, 602, 603, 605, 607, 609, 616, 627, 630, 631, 635, 636, 638, 639, 640, 641, 642, 643, 644, 651, 676, 683, 688, 690, 694, 698, 699, 706, 711, 713, 1719, 720, 744, 749, 754, 756, 760, , 781, 786, 797, 810, 811, 812, 815, 823, 824, 825, 827, 828, 833, 834, 835, 837, 843, 848, 868, 869, 870, 871, 872, 873, 874, 880, 881, 883, 884, 885, 887, 888, 890, 892, 894, 898, 905, 906, 912, 920, 921, 924, 926, 927, 928, 932, 933, 934, 935, 937, 938, 939, 940, 941, 942, 943, 946, 948, 950, 952, 953, 954, 956, 957, 960, 966, 971, 972, 980, 989, 991, 994, 995, 998, 999, 1006, 1009, 1010, 1011, 1029, 1030, 1031, 1032, 1035, 1037, 1038, 1040, 1041, 1042, 1044, 1045, 1048, wherein numbering is according to SEQ ID NO:2.

In another aspect, a variant comprises an alteration at each position (or at least four positions) corresponding to positions 17, 20, 51, 53, 55, 56, 60, 63, 79, 87, 192, 302, 387, 388, 390, 403, 408, 410, 416, 448, 451, 471, 472, 507, 512, 515, 538, 598, 602, 605, 609, 676, 694, 698, 699, 711, 754, 760, , 781, 786, 797, 834, and 835 of SEQ ID NO:2.

In another aspect, the variant comprises or consists of an alteration at a position corresponding to position 4. In another aspect, the amino acid at a position corresponding to position 4 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution V4T of the mature polypeptide of SEQ ID NO:2.

In another aspect, the variant comprises or consists of an alteration at a position corresponding to position 17. In another aspect, the amino acid at a position corresponding to position 17 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution S17A of the mature polypeptide of SEQ ID NO:2.

In another aspect, the variant comprises or consists of an alteration at a position corresponding to position 18. In another aspect, the amino acid at a position corresponding to position 18 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution N18G of the mature polypeptide of SEQ ID NO:2.

In another aspect, the variant comprises or consists of an alteration at a position corresponding to position 20. In another aspect, the amino acid at a position corresponding to position 20 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution F20P, F20N, F20G, or F20Y, preferably F20P, of the mature polypeptide of SEQ ID NO:2.

In another aspect, the variant comprises or consists of an alteration at a position corresponding to position 51. In another aspect, the amino acid at a position corresponding to position 51 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K51Q or K51H, preferably K51Q, of the mature polypeptide of SEQ ID NO:2.

In another aspect, the variant comprises or consists of an alteration at a position corresponding to position 53. In another aspect, the amino acid at a position corresponding to position 53 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution E53Y, E53P, or E53G, preferably E53Y, of the mature polypeptide of SEQ ID NO:2.

In another aspect, the variant comprises or consists of an alteration at a position corresponding to position 55. In another aspect, the amino acid at a position corresponding to position 55 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution Y55M or Y55D, preferably Y55M, of the mature polypeptide of SEQ ID NO:2.

In another aspect, the variant comprises or consists of an alteration at a position corresponding to position 56. In another aspect, the amino acid at a position corresponding to position 56 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution V56M of the mature polypeptide of SEQ ID NO:2.

In another aspect, the variant comprises or consists of an alteration at a position corresponding to position 60. In another aspect, the amino acid at a position corresponding to position 60 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution Y6OF of the mature polypeptide of SEQ ID NO:2.

In another aspect, the variant comprises or consists of an alteration at a position corresponding to position 63. In another aspect, the amino acid at a position corresponding to position 63 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution S63F of the mature polypeptide of SEQ ID NO:2.

In another aspect, the variant comprises or consists of an alteration at a position corresponding to position 71. In another aspect, the amino acid at a position corresponding to position 71 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A71E of the mature polypeptide of SEQ ID NO:2.

In another aspect, the variant comprises or consists of an alteration at a position corresponding to position 79. In another aspect, the amino acid at a position corresponding to position 79 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution S79W of the mature polypeptide of SEQ ID NO:2.

In another aspect, the variant comprises or consists of an alteration at a position corresponding to position 87. In another aspect, the amino acid at a position corresponding to position 87 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T87R of the mature polypeptide of SEQ ID NO:2.

In another aspect, the variant comprises or consists of an alteration at a position corresponding to position 92. In another aspect, the amino acid at a position corresponding to position 92 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution T92S of the mature polypeptide of SEQ ID NO:2.

In another aspect, the variant comprises or consists of an alteration at a position corresponding to position 120. In another aspect, the amino acid at a position corresponding to position 120 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A120P of the mature polypeptide of SEQ ID NO:2.

In another aspect, the variant comprises or consists of an alteration at a position corresponding to position 129. In another aspect, the amino acid at a position corresponding to position 129 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution N129D of the mature polypeptide of SEQ ID NO:2.

In another aspect, the variant comprises or consists of an alteration at a position corresponding to position 137. In another aspect, the amino acid at a position corresponding to position 137 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution F137L of the mature polypeptide of SEQ ID NO:2.

In another aspect, the variant comprises or consists of an alteration at a position corresponding to position 182. In another aspect, the amino acid at a position corresponding to position 182 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution H182Y of the mature polypeptide of SEQ ID NO:2.

In another aspect, the variant comprises or consists of an alteration at a position corresponding to position 186. In another aspect, the amino acid at a position corresponding to position 186 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A186P of the mature polypeptide of SEQ ID NO:2.

In another aspect, the variant comprises or consists of an alteration at a position corresponding to position 189. In another aspect, the amino acid at a position corresponding to position 189 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution N189K of the mature polypeptide of SEQ ID NO:2.

In another aspect, the variant comprises or consists of an alteration at a position corresponding to position 192. In another aspect, the amino acid at a position corresponding to position 192 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K192N of the mature polypeptide of SEQ ID NO:2.

In another aspect, the variant comprises or consists of an alteration at a position corresponding to position 216. In another aspect, the amino acid at a position corresponding to position 216 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution N216D, N216Q, r N216R, preferably N216D, of the mature polypeptide of SEQ ID NO:2.

In another aspect, the variant comprises or consists of an alteration at a position corresponding to position 226. In another aspect, the amino acid at a position corresponding to position 226 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution L226K of the mature polypeptide of SEQ ID NO:2.

In another aspect, the variant comprises or consists of an alteration at a position corresponding to position 228. In another aspect, the amino acid at a position corresponding to position 228 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K228E of the mature polypeptide of SEQ ID NO:2.

In another aspect, the variant comprises or consists of an alteration at a position corresponding to position 230. In another aspect, the amino acid at a position corresponding to position 230 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution G230H of the mature polypeptide of SEQ ID NO:2.

In another aspect, the variant comprises or consists of an alteration at a position corresponding to position 233. In another aspect, the amino acid at a position corresponding to position 233 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution L233H of the mature polypeptide of SEQ ID NO:2.

In another aspect, the variant comprises or consists of an alteration at a position corresponding to position 247. In another aspect, the amino acid at a position corresponding to position 247 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution D247N of the mature polypeptide of SEQ ID NO:2.

In another aspect, the variant comprises or consists of an alteration at a position corresponding to position 278. In another aspect, the amino acid at a position corresponding to position 278 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A278S of the mature polypeptide of SEQ ID NO:2.

In another aspect, the variant comprises or consists of an alteration at a position corresponding to position 279. In another aspect, the amino acid at a position corresponding to position 279 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution G279E of the mature polypeptide of SEQ ID NO:2.

In another aspect, the variant comprises or consists of an alteration at a position corresponding to position 281. In another aspect, the amino acid at a position corresponding to position 281 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution K281R of the mature polypeptide of SEQ ID NO:2.

In another aspect, the variant comprises or consists of an alteration at a position corresponding to position 283. In another aspect, the amino acid at a position corresponding to position 283 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A283D of the mature polypeptide of SEQ ID NO:2.

In another aspect, the variant comprises or consists of an alteration at a position corresponding to position 285. In another aspect, the amino acid at a position corresponding to position 285 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution N285L, N285M, N285S, N285P, N285T, N285Y, N285H, N285K, N285D, N285W, N285R, or N285G, preferably N285G, of the mature polypeptide of SEQ ID NO:2.

In another aspect, the variant comprises or consists of an alteration at a position corresponding to position 289. In another aspect, the amino acid at a position corresponding to position 289 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution Q289E of the mature polypeptide of SEQ ID NO:2.

In another aspect, the variant comprises or consists of an alteration at a position corresponding to position 292. In another aspect, the amino acid at a position corresponding to position 292 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T292F, T292L, T2921, T292V, T292S, T292P, T292Y, T292Q, T292N, T292K, T292D, T292A, or T292G, preferably T292A, of the mature polypeptide of SEQ ID NO:2.

In another aspect, the variant comprises or consists of an alteration at a position corresponding to position 294. In another aspect, the amino acid at a position corresponding to position 294 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A294V of the mature polypeptide of SEQ ID NO:2.

In another aspect, the variant comprises or consists of an alteration at a position corresponding to position 297. In another aspect, the amino acid at a position corresponding to position 297 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution F297L of the mature polypeptide of SEQ ID NO:2.

In another aspect, the variant comprises or consists of an alteration at a position corresponding to position 298. In another aspect, the amino acid at a position corresponding to position 298 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution Q298E of the mature polypeptide of SEQ ID NO:2.

In another aspect, the variant comprises or consists of an alteration at a position corresponding to position 302. In another aspect, the amino acid at a position corresponding to position 302 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution I302D, I302H, I302V, or I302M, preferably I302D, of the mature polypeptide of SEQ ID NO:2.

In another aspect, the variant comprises or consists of an alteration at a position corresponding to position 311. In another aspect, the amino acid at a position corresponding to position 311 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution H311N of the mature polypeptide of SEQ ID NO:2.

In another aspect, the variant comprises or consists of an alteration at a position corresponding to position 313. In another aspect, the amino acid at a position corresponding to position 313 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution S313D of the mature polypeptide of SEQ ID NO:2.

In another aspect, the variant comprises or consists of an alteration at a position corresponding to position 333. In another aspect, the amino acid at a position corresponding to position 333 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution W333L of the mature polypeptide of SEQ ID NO:2.

In another aspect, the variant comprises or consists of an alteration at a position corresponding to position 346. In another aspect, the amino acid at a position corresponding to position 346 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A346H or A246D of the mature polypeptide of SEQ ID NO:2.

In another aspect, the variant comprises or consists of an alteration at a position corresponding to position 353. In another aspect, the amino acid at a position corresponding to position 353 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution T353D of the mature polypeptide of SEQ ID NO:2.

In another aspect, the variant comprises or consists of an alteration at a position corresponding to position 386. In another aspect, the amino acid at a position corresponding to position 386 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A386P of the mature polypeptide of SEQ ID NO:2.

In another aspect, the variant comprises or consists of an alteration at a position corresponding to position 387. In another aspect, the amino acid at a position corresponding to position 387 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution I387T of the mature polypeptide of SEQ ID NO:2.

In another aspect, the variant comprises or consists of an alteration at a position corresponding to position 388. In another aspect, the amino acid at a position corresponding to position 388 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K388R of the mature polypeptide of SEQ ID NO:2.

In another aspect, the variant comprises or consists of an alteration at a position corresponding to position 390. In another aspect, the amino acid at a position corresponding to position 390 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K390Q of the mature polypeptide of SEQ ID NO:2.

In another aspect, the variant comprises or consists of an alteration at a position corresponding to position 403. In another aspect, the amino acid at a position corresponding to position 403 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution I403Y of the mature polypeptide of SEQ ID NO:2.

In another aspect, the variant comprises or consists of an alteration at a position corresponding to position 408 In another aspect, the amino acid at a position corresponding to position 408 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution E408D, E408N, E4085, E408P, E408A, E408G, or E408G, preferably E408D, of the mature polypeptide of SEQ ID NO:2.

In another aspect, the variant comprises or consists of an alteration at a position corresponding to position 410. In another aspect, the amino acid at a position corresponding to position 410 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution P410G of the mature polypeptide of SEQ ID NO:2.

In another aspect, the variant comprises or consists of an alteration at a position corresponding to position 416. In another aspect, the amino acid at a position corresponding to position 416 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution Q416S or Q416D, preferably Q416S, of the mature polypeptide of SEQ ID NO:2.

In another aspect, the variant comprises or consists of an alteration at a position corresponding to position 441. In another aspect, the amino acid at a position corresponding to position 441 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution N441G of the mature polypeptide of SEQ ID NO:2.

In another aspect, the variant comprises or consists of an alteration at a position corresponding to position 448. In another aspect, the amino acid at a position corresponding to position 448 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A448E, A448W, or A448S, preferably A448E, of the mature polypeptide of SEQ ID NO:2.

In another aspect, the variant comprises or consists of an alteration at a position corresponding to position 451. In another aspect, the amino acid at a position corresponding to position 451 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution K451S or K451Q, preferably K451S or preferably K451Q, of the mature polypeptide of SEQ ID NO:2.

In another aspect, the variant comprises or consists of an alteration at a position corresponding to position 471. In another aspect, the amino acid at a position corresponding to position 471 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution G471S of the mature polypeptide of SEQ ID NO:2.

In another aspect, the variant comprises or consists of an alteration at a position corresponding to position 472. In another aspect, the amino acid at a position corresponding to position 472 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution S472Y of the mature polypeptide of SEQ ID NO:2.

In another aspect, the variant comprises or consists of an alteration at a position corresponding to position 476. In another aspect, the amino acid at a position corresponding to position 476 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution D476R of the mature polypeptide of SEQ ID NO:2.

In another aspect, the variant comprises or consists of an alteration at a position corresponding to position 489. In another aspect, the amino acid at a position corresponding to position 489 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution Q489P of the mature polypeptide of SEQ ID NO:2.

In another aspect, the variant comprises or consists of an alteration at a position corresponding to position 507. In another aspect, the amino acid at a position corresponding to position 507 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K507R of the mature polypeptide of SEQ ID NO:2.

In another aspect, the variant comprises or consists of an alteration at a position corresponding to position 512. In another aspect, the amino acid at a position corresponding to position 512 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K512P of the mature polypeptide of SEQ ID NO:2.

In another aspect, the variant comprises or consists of an alteration at a position corresponding to position 515. In another aspect, the amino acid at a position corresponding to position 515 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution S515V of the mature polypeptide of SEQ ID NO:2.

In another aspect, the variant comprises or consists of an alteration at a position corresponding to position 538. In another aspect, the amino acid at a position corresponding to position 538 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution S538C of the mature polypeptide of SEQ ID NO:2.

In another aspect, the variant comprises or consists of an alteration at a position corresponding to position 555. In another aspect, the amino acid at a position corresponding to position 555 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution L555Q of the mature polypeptide of SEQ ID NO:2.

In another aspect, the variant comprises or consists of an alteration at a position corresponding to position 557. In another aspect, the amino acid at a position corresponding to position 557 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution G557R of the mature polypeptide of SEQ ID NO:2.

In another aspect, the variant comprises or consists of an alteration at a position corresponding to position 558. In another aspect, the amino acid at a position corresponding to position 558 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the alteration N558D, N558NP, N558F, N5581, N558E, or N558M of the mature polypeptide of SEQ ID NO:2.

In another aspect, the variant comprises or consists of an alteration at a position corresponding to position 559. In another aspect, the amino acid at a position corresponding to position 559 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A559S, A559N, A559F, A559M, A559P, A559Y, A559H, A559Q, A559D, or A559G, preferably A559N, of the mature polypeptide of SEQ ID NO:2.

In another aspect, the variant comprises or consists of an alteration at a position corresponding to position 560. In another aspect, the amino acid at a position corresponding to position 560 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution S560P and S560G, preferably S560P, of the mature polypeptide of SEQ ID NO:2.

In another aspect, the variant comprises or consists of an alteration at a position corresponding to position 561. In another aspect, the amino acid ata position corresponding to position 561 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T561P of the mature polypeptide of SEQ ID NO:2.

In another aspect, the variant comprises or consists of an alteration at a position corresponding to position 564. In another aspect, the amino acid at a position corresponding to position 564 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A564I, A564Y, A564Q, A564E, or A564K, preferably A564I, of the mature polypeptide of SEQ ID NO:2.

In another aspect, the variant comprises or consists of an alteration at a position corresponding to position 567. In another aspect, the amino acid at a position corresponding to position 567 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution V567F or V567P of the mature polypeptide of SEQ ID NO:2.

In another aspect, the variant comprises or consists of an alteration at a position corresponding to position 568. In another aspect, the amino acid at a position corresponding to position 568 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution K568R of the mature polypeptide of SEQ ID NO:2.

In another aspect, the variant comprises or consists of an alteration at a position corresponding to position 570. In another aspect, the amino acid at a position corresponding to position 570 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution P570K, P570Q, P570R, P570T, P570S, P570A, P570H, P570G, and P570N, preferably P570K or P570R, of the mature polypeptide of SEQ ID NO:2.

In another aspect, the variant comprises or consists of an alteration at a position corresponding to position 575. In another aspect, the amino acid at a position corresponding to position 575 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution I575V of the mature polypeptide of SEQ ID NO:2.

In another aspect, the variant comprises or consists of an alteration at a position corresponding to position 579. In another aspect, the amino acid at a position corresponding to position 579 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution Y579W or Y579F, preferably Y579W, of the mature polypeptide of SEQ ID NO:2.

In another aspect, the variant comprises or consists of an alteration at a position corresponding to position 581. In another aspect, the amino acid at a position corresponding to position 581 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution T581M of the mature polypeptide of SEQ ID NO:2.

In another aspect, the variant comprises or consists of an alteration at a position corresponding to position 592. In another aspect, the amino acid at a position corresponding to position 592 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution G592D of the mature polypeptide of SEQ ID NO:2.

In another aspect, the variant comprises or consists of an alteration at a position corresponding to position 593. In another aspect, the amino acid at a position corresponding to position 593 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution S593N and S593E, preferably S593N, of the mature polypeptide of SEQ ID NO:2.

In another aspect, the variant comprises or consists of an alteration at a position corresponding to position 595. In another aspect, the amino acid at a position corresponding to position 595 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution S595L of the mature polypeptide of SEQ ID NO:2.

In another aspect, the variant comprises or consists of an alteration at a position corresponding to position 598. In another aspect, the amino acid at a position corresponding to position 598 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution S598Q of the mature polypeptide of SEQ ID NO:2.

In another aspect, the variant comprises or consists of an alteration at a position corresponding to position 599. In another aspect, the amino acid at a position corresponding to position 599 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A599S of the mature polypeptide of SEQ ID NO:2.

In another aspect, the variant comprises or consists of an alteration at a position corresponding to position 602. In another aspect, the amino acid at a position corresponding to position 602 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution I602T or I602D, preferably I602T, of the mature polypeptide of SEQ ID NO:2.

In another aspect, the variant comprises or consists of an alteration at a position corresponding to position 603. In another aspect, the amino acid at a position corresponding to position 603 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution V603P of the mature polypeptide of SEQ ID NO:2.

In another aspect, the variant comprises or consists of an alteration at a position corresponding to position 605. In another aspect, the amino acid at a position corresponding to position 605 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution S605T of the mature polypeptide of SEQ ID NO:2.

In another aspect, the variant comprises or consists of an alteration at a position corresponding to position 607. In another aspect, the amino acid at a position corresponding to position 607 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution S607C of the mature polypeptide of SEQ ID NO:2.

In another aspect, the variant comprises or consists of an alteration at a position corresponding to position 609. In another aspect, the amino acid at a position corresponding to position 609 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution G609E of the mature polypeptide of SEQ ID NO:2.

In another aspect, the variant comprises or consists of an alteration at a position corresponding to position 616. In another aspect, the amino acid at a position corresponding to position 616 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution S616D or S616G, preferably S616D, of the mature polypeptide of SEQ ID NO:2.

In another aspect, the variant comprises or consists of an alteration at a position corresponding to position 627. In another aspect, the amino acid at a position corresponding to position 627 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution K627L, K627M, K627V, K627S, K627T, K627Q, or K627R, preferably K627R, of the mature polypeptide of SEQ ID NO:2.

In another aspect, the variant comprises or consists of an alteration at a position corresponding to position 630. In another aspect, the amino acid at a position corresponding to position 630 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution I630F, I630V, or I630Y of the mature polypeptide of SEQ ID NO:2.

In another aspect, the variant comprises or consists of an alteration at a position corresponding to position 631. In another aspect, the amino acid at a position corresponding to position 631 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K631A or K631R, preferably K631R, of the mature polypeptide of SEQ ID NO:2.

In another aspect, the variant comprises or consists of an alteration at a position corresponding to position 633. In another aspect, the amino acid at a position corresponding to position 633 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution T633V of the mature polypeptide of SEQ ID NO:2.

In another aspect, the variant comprises or consists of an alteration at a position corresponding to position 635. In another aspect, the amino acid at a position corresponding to position 635 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution D635P, D635N, D635K, D635E, D635W, D635L, D635M, D635T, D635A, or D635G, preferably D635A, of the mature polypeptide of SEQ ID NO:2.

In another aspect, the variant comprises or consists of an alteration at a position corresponding to position 636. In another aspect, the amino acid at a position corresponding to position 636 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution S636M, S636A, S636H, S636Q, S636N, S636R, S636L, S636H, or S636K, preferably S636N, of the mature polypeptide of SEQ ID NO:2.

In another aspect, the variant comprises or consists of an alteration at a position corresponding to position 638. In another aspect, the amino acid at a position corresponding to position 638 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution F638N, F6381, F638V, F638T, F638L, F638Y, F638M or F638H, preferably F638N, of the mature polypeptide of SEQ ID NO:2.

In another aspect, the variant comprises or consists of an alteration at a position corresponding to position 639. In another aspect, the amino acid at a position corresponding to position 639 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T639S, T6391, T639M, T639V, T639A, T639D, T639E, T639Y, T639W, T639P, or T639G, preferably T639G or T6391, of the mature polypeptide of SEQ ID NO:2.

In another aspect, the variant comprises or consists of an alteration at a position corresponding to position 640. In another aspect, the amino acid at a position corresponding to position 640 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T640S of the mature polypeptide of SEQ ID NO:2.

In another aspect, the variant comprises or consists of an alteration at a position corresponding to position 642. In another aspect, the amino acid at a position corresponding to position 642 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution S642T or S642N, preferably S642N, of the mature polypeptide of SEQ ID NO:2.

In another aspect, the variant comprises or consists of an alteration at a position corresponding to position 643. In another aspect, the amino acid at a position corresponding to position 643 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution N643D or N643H, preferably N643D, of the mature polypeptide of SEQ ID NO:2.

In another aspect, the variant comprises or consists of an alteration at a position corresponding to position 651. In another aspect, the amino acid at a position corresponding to position 651 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A651P or A651S, preferably A651P, of the mature polypeptide of SEQ ID NO:2.

In another aspect, the variant comprises or consists of an alteration at a position corresponding to position 676. In another aspect, the amino acid at a position corresponding to position 676 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution D676H of the mature polypeptide of SEQ ID NO:2.

In another aspect, the variant comprises or consists of an alteration at a position corresponding to position 683. In another aspect, the amino acid at a position corresponding to position 683 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution Q683E of the mature polypeptide of SEQ ID NO:2.

In another aspect, the variant comprises or consists of an alteration at a position corresponding to position 688. In another aspect, the amino acid at a position corresponding to position 688 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A688G of the mature polypeptide of SEQ ID NO:2.

In another aspect, the variant comprises or consists of an alteration at a position corresponding to position 690. In another aspect, the amino acid at a position corresponding to position 690 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution Y690F of the mature polypeptide of SEQ ID NO:2.

In another aspect, the variant comprises or consists of an alteration at a position corresponding to position 694. In another aspect, the amino acid at a position corresponding to position 694 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T694A of the mature polypeptide of SEQ ID NO:2.

In another aspect, the variant comprises or consists of an alteration at a position corresponding to position 697. In another aspect, the amino acid at a position corresponding to position 697 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T697G of the mature polypeptide of SEQ ID NO:2.

In another aspect, the variant comprises or consists of an alteration at a position corresponding to position 698. In another aspect, the amino acid at a position corresponding to position 698 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution R698W of the mature polypeptide of SEQ ID NO:2.

In another aspect, the variant comprises or consists of an alteration at a position corresponding to position 699. In another aspect, the amino acid at a position corresponding to position 699 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T699A of the mature polypeptide of SEQ ID NO:2.

In another aspect, the variant comprises or consists of an alteration at a position corresponding to position 706. In another aspect, the amino acid at a position corresponding to position 706 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T706Q of the mature polypeptide of SEQ ID NO:2.

In another aspect, the variant comprises or consists of an alteration at a position corresponding to position 711. In another aspect, the amino acid at a position corresponding to position 711 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T711S, T711V, or T711Y, preferably T711V, of the mature polypeptide of SEQ ID NO:2.

In another aspect, the variant comprises or consists of an alteration at a position corresponding to position 713. In another aspect, the amino acid at a position corresponding to position 713 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution K713R of the mature polypeptide of SEQ ID NO:2.

In another aspect, the variant comprises or consists of an alteration at a position corresponding to position 719. In another aspect, the amino acid at a position corresponding to position 719 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution W719R of the mature polypeptide of SEQ ID NO:2.

In another aspect, the variant comprises or consists of an alteration at a position corresponding to position 720. In another aspect, the amino acid at a position corresponding to position 720 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution K720H of the mature polypeptide of SEQ ID NO:2.

In another aspect, the variant comprises or consists of an alteration at a position corresponding to position 744. In another aspect, the amino acid at a position corresponding to position 744 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K744H or K744Q, preferably K744H, of the mature polypeptide of SEQ ID NO:2.

In another aspect, the variant comprises or consists of an alteration at a position corresponding to position 749. In another aspect, the amino acid at a position corresponding to position 749 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A749T of the mature polypeptide of SEQ ID NO:2.

In another aspect, the variant comprises or consists of an alteration at a position corresponding to position 754. In another aspect, the amino acid at a position corresponding to position 754 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K754R of the mature polypeptide of SEQ ID NO:2.

In another aspect, the variant comprises or consists of an alteration at a position corresponding to position 756. In another aspect, the amino acid at a position corresponding to position 756 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution V756Y or V756H, preferably V756Y, of the mature polypeptide of SEQ ID NO:2.

In another aspect, the variant comprises or consists of an alteration at a position corresponding to position 760. In another aspect, the amino acid at a position corresponding to position 760 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution S760G of the mature polypeptide of SEQ ID NO:2.

In another aspect, the variant comprises or consists of an alteration at a position corresponding to position , 781. In another aspect, the amino acid at a position corresponding to position , 781 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution T, 781M of the mature polypeptide of SEQ ID NO:2.

In another aspect, the variant comprises or consists of an alteration at a position corresponding to position 786. In another aspect, the amino acid at a position corresponding to position 786 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution N786K of the mature polypeptide of SEQ ID NO:2.

In another aspect, the variant comprises or consists of an alteration at a position corresponding to position 797. In another aspect, the amino acid at a position corresponding to position 797 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution T797S of the mature polypeptide of SEQ ID NO:2.

In another aspect, the variant comprises or consists of an alteration at a position corresponding to position 810. In another aspect, the amino acid at a position corresponding to position 810 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution S810Q of the mature polypeptide of SEQ ID NO:2.

In another aspect, the variant comprises or consists of an alteration at a position corresponding to position 824. In another aspect, the amino acid at a position corresponding to position 824 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A824D of the mature polypeptide of SEQ ID NO:2.

In another aspect, the variant comprises or consists of an alteration at a position corresponding to position 825. In another aspect, the amino acid at a position corresponding to position 825 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T825G of the mature polypeptide of SEQ ID NO:2.

In another aspect, the variant comprises or consists of an alteration at a position corresponding to position 828. In another aspect, the amino acid at a position corresponding to position 828 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution N828D of the mature polypeptide of SEQ ID NO:2.

In another aspect, the variant comprises or consists of an alteration at a position corresponding to position 833. In another aspect, the amino acid at a position corresponding to position 833 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution N833D of the mature polypeptide of SEQ ID NO:2.

In another aspect, the variant comprises or consists of an alteration at a position corresponding to position 834. In another aspect, the amino acid at a position corresponding to position 834 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution Q834E of the mature polypeptide of SEQ ID NO:2.

In another aspect, the variant comprises or consists of an alteration at a position corresponding to position 835. In another aspect, the amino acid at a position corresponding to position 835 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution S835A or S835D, preferably S835A, of the mature polypeptide of SEQ ID NO:2.

In another aspect, the variant comprises or consists of an alteration at a position corresponding to position 837. In another aspect, the amino acid at a position corresponding to position 837 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution V837I of the mature polypeptide of SEQ ID NO:2.

In another aspect, the variant comprises or consists of an alteration at a position corresponding to position 848. In another aspect, the amino acid at a position corresponding to position 848 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution N848D of the mature polypeptide of SEQ ID NO:2.

In another aspect, the variant comprises or consists of an alteration at a position corresponding to position 868. In another aspect, the amino acid at a position corresponding to position 868 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A868E of the mature polypeptide of SEQ ID NO:2.

In another aspect, the variant comprises or consists of an alteration at a position corresponding to position 869. In another aspect, the amino acid at a position corresponding to position 869 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A869V of the mature polypeptide of SEQ ID NO:2.

In another aspect, the variant comprises or consists of an alteration at a position corresponding to position 870. In another aspect, the amino acid at a position corresponding to position 870 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution D870V of the mature polypeptide of SEQ ID NO:2.

In another aspect, the variant comprises or consists of an alteration at a position corresponding to position 872. In another aspect, the amino acid at a position corresponding to position 872 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution T872G, T872H, T872W, or T872Q, preferably T872G, of the mature polypeptide of SEQ ID NO:2.

In another aspect, the variant comprises or consists of an alteration at a position corresponding to position 880. In another aspect, the amino acid at a position corresponding to position 880 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution R880K of the mature polypeptide of SEQ ID NO:2.

In another aspect, the variant comprises or consists of an alteration at a position corresponding to position 881. In another aspect, the amino acid at a position corresponding to position 881 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution V881Q or V881T, preferably V881Q, of the mature polypeptide of SEQ ID NO:2.

In another aspect, the variant comprises or consists of an alteration at a position corresponding to position 883. In another aspect, the amino acid at a position corresponding to position 883 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution T883R, T883V, T883C, or T883K, preferably T883R, of the mature polypeptide of SEQ ID NO:2.

In another aspect, the variant comprises or consists of an alteration at a position corresponding to position 884. In another aspect, the amino acid at a position corresponding to position 884 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution Y884H of the mature polypeptide of SEQ ID NO:2.

In another aspect, the variant comprises or consists of an alteration at a position corresponding to position 885. In another aspect, the amino acid at a position corresponding to position 885 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A885Q, A885N, or A885F, preferably A885F, of the mature polypeptide of SEQ ID NO:2.

In another aspect, the variant comprises or consists of an alteration at a position corresponding to position 887. In another aspect, the amino acid at a position corresponding to position 887 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T887S or T887K, preferably T887K, of the mature polypeptide of SEQ ID NO:2.

In another aspect, the variant comprises or consists of an alteration at a position corresponding to position 888. In another aspect, the amino acid at a position corresponding to position 888 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution L888M of the mature polypeptide of SEQ ID NO:2.

In another aspect, the variant comprises or consists of an alteration at a position corresponding to position 890. In another aspect, the amino acid at a position corresponding to position 890 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution V890R of the mature polypeptide of SEQ ID NO:2.

In another aspect, the variant comprises or consists of an alteration at a position corresponding to position 892. In another aspect, the amino acid at a position corresponding to position 892 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T892V or T892P, preferably T892P, of the mature polypeptide of SEQ ID NO:2.

In another aspect, the variant comprises or consists of an alteration at a position corresponding to position 898. In another aspec, the amino acid at a position corresponding to position 898 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution R898Q of the mature polypeptide of SEQ ID NO:2.

In another aspect, the variant comprises or consists of an alteration at a position corresponding to position 905. In another aspect, the amino acid at a position corresponding to position 905 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution N905D of the mature polypeptide of SEQ ID NO:2.

In another aspect, the variant comprises or consists of an alteration at a position corresponding to position 906. In another aspect, the amino acid at a position corresponding to position 906 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution F906A of the mature polypeptide of SEQ ID NO:2.

In another aspect, the variant comprises or consists of an alteration at a position corresponding to position 912. In another aspect, the amino acid at a position corresponding to position 912 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution Q912V of the mature polypeptide of SEQ ID NO:2.

In another aspect, the variant comprises or consists of an alteration at a position corresponding to position 920. In another aspect, the amino acid at a position corresponding to position 920 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution N920D or N920P of the mature polypeptide of SEQ ID NO:2.

In another aspect, the variant comprises or consists of an alteration at a position corresponding to position 921. In another aspect, the amino acid at a position corresponding to position 921 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution K921R or K921E of the mature polypeptide of SEQ ID NO:2.

In another aspect, the variant comprises or consists of an alteration at a position corresponding to position 924. In another aspect, the amino acid at a position corresponding to position 924 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A924D of the mature polypeptide of SEQ ID NO:2.

In another aspect, the variant comprises or consists of an alteration at a position corresponding to position 926. In another aspect, the amino acid at a position corresponding to position 926 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution V926F or V926P, preferably V926P, of the mature polypeptide of SEQ ID NO:2.

In another aspect, the variant comprises or consists of an alteration at a position corresponding to position 927. In another aspect, the amino acid at a position corresponding to position 927 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution K927R of the mature polypeptide of SEQ ID NO:2.

In another aspect, the variant comprises or consists of an alteration at a position corresponding to position 928. In another aspect, the amino acid at a position corresponding to position 928 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution S928D of the mature polypeptide of SEQ ID NO:2.

In another aspect, the variant comprises or consists of an alteration at a position corresponding to position 932. In another aspect, the amino acid at a position corresponding to position 932 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution T932A of the mature polypeptide of SEQ ID NO:2.

In another aspect, the variant comprises or consists of an alteration at a position corresponding to position 933. In another aspect, the amino acid at a position corresponding to position 933 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution N933S or N933V, preferably N933S, of the mature polypeptide of SEQ ID NO:2.

In another aspect, the variant comprises or consists of an alteration at a position corresponding to position 934. In another aspect, the amino acid at a position corresponding to position 934 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution Y934G, Y034R, or Y934Q, preferably Y934G, of the mature polypeptide of SEQ ID NO:2.

In another aspect, the variant comprises or consists of an alteration at a position corresponding to position 937. In another aspect, the amino acid at a position corresponding to position 937 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A937E of the mature polypeptide of SEQ ID NO:2.

In another aspect, the variant comprises or consists of an alteration at a position corresponding to position 938. In another aspect, the amino acid at a position corresponding to position 938 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution V938I of the mature polypeptide of SEQ ID NO:2.

In another aspect, the variant comprises or consists of an alteration at a position corresponding to position 939. In another aspect, the amino acid at a position corresponding to position 939 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution K939V of the mature polypeptide of SEQ ID NO:2.

In another aspect, the variant comprises or consists of an alteration at a position corresponding to position 941. In another aspect, the amino acid at a position corresponding to position 941 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution N941S of the mature polypeptide of SEQ ID NO:2.

In another aspect, the variant comprises or consists of an alteration at a position corresponding to position 942. In another aspect, the amino acid at a position corresponding to position 942 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A942P of the mature polypeptide of SEQ ID NO:2.

In another aspect, the variant comprises or consists of an alteration at a position corresponding to position 946. In another aspect, the amino acid at a position corresponding to position 946 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution G946R of the mature polypeptide of SEQ ID NO:2.

In another aspect, the variant comprises or consists of an alteration at a position corresponding to position 948. In another aspect, the amino acid at a position corresponding to position 948 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution K948R of the mature polypeptide of SEQ ID NO:2.

In another aspect, the variant comprises or consists of an alteration at a position corresponding to position 956. In another aspect, the amino acid at a position corresponding to position 956 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution Q956Y or A956S, preferably Q956Y, of the mature polypeptide of SEQ ID NO:2.

In another aspect, the variant comprises or consists of an alteration at a position corresponding to position 957. In another aspect, the amino acid at a position corresponding to position 957 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A957L or A957P, preferably A957L, of the mature polypeptide of SEQ ID NO:2.

In another aspect, the variant comprises or consists of an alteration at a position corresponding to position 966. In another aspect, the amino acid at a position corresponding to position 966 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution N966C of the mature polypeptide of SEQ ID NO:2.

In another aspect, the variant comprises or consists of an alteration at a position corresponding to position 972. In another aspect, the amino acid at a position corresponding to position 972 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T972K of the mature polypeptide of SEQ ID NO:2.

In another aspect, the variant comprises or consists of an alteration at a position corresponding to position 980. In another aspect, the amino acid at a position corresponding to position 980 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution M980I of the mature polypeptide of SEQ ID NO:2.

In another aspect, the variant comprises or consists of an alteration at a position corresponding to position 994. In another aspect, the amino acid at a position corresponding to position 994 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution G994N or G994D of the mature polypeptide of SEQ ID NO:2.

In another aspect, the variant comprises or consists of an alteration at a position corresponding to position 999. In another aspect, the amino acid at a position corresponding to position 999 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution T999R of the mature polypeptide of SEQ ID NO:2.

In another aspect, the variant comprises or consists of an alteration at a position corresponding to position 1011. In another aspect, the amino acid at a position corresponding to position 1011 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution L1011A of the mature polypeptide of SEQ ID NO:2.

In another aspect, the variant comprises or consists of an alteration at a position corresponding to position 1031. In another aspect, the amino acid at a position corresponding to position 1031 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K1031I of the mature polypeptide of SEQ ID NO:2.

In another aspect, the variant comprises or consists of an alteration at a position corresponding to position 1037. In another aspect, the amino acid at a position corresponding to position 1037 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A1037E of the mature polypeptide of SEQ ID NO:2.

In another aspect, the variant comprises or consists of an alteration at a position corresponding to position 1038. In another aspect, the amino acid at a position corresponding to position 1038 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution S1038G of the mature polypeptide of SEQ ID NO:2.

In another aspect, the variant comprises or consists of an alteration at a position corresponding to position 1041. In another aspect, the amino acid at a position corresponding to position 1041 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution G1041R of the mature polypeptide of SEQ ID NO:2.

In another aspect, the variant comprises or consists of an alteration at a position corresponding to position 1042. In another aspect, the amino acid at a position corresponding to position 1042 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution Y1042N of the mature polypeptide of SEQ ID NO:2.

In another aspect, the variant comprises or consists of an alteration at a position corresponding to position 1048. In another aspect, the amino acid at a position corresponding to position 1048 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution F1048W of the mature polypeptide of SEQ ID NO:2.

In another aspect, the variant comprises or consists of alterations at positions corresponding to positions 559+579. In another aspect, the amino acids at positions corresponding to positions 559+579 are idependently substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitutions A559N+Y579W or A559N+Y579F of the mature polypeptide of SEQ ID NO:2.

In another aspect, the variant comprises or consists of an alteration ata position corresponding to position selected from the group consisting of alterations in positions: 17, 20, 51, 53, 55, 56, 60, 63, 79, 87, 192, 302, 387, 388, 390, 403, 408, 410, 416, 448, 451, 471, 472, 507, 512, 515, 538, 598, 602, 605, 609, 676, 694, 698, 699, 711, 754, 760, , 781, 786, 797, 834, and 835 of SEQ ID NO:2. In another aspect, the amino acid at a position corresponding to any of positions as described above is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution selected from the group consisting of: S17A, F20P, F20N, F20G, F20Y, K51Q, K51H, E53P, E53G, Y55M, V56M, Y60F, S63F, T87R, K192N, I302H, I302V, I302M, I387T, K388R, K390Q, I403Y, E408D, E4085, E408P, E408A, E408G, E408N, P410G, Q4165, Q416D, A448E, A448W, A4485, K451S, G471S, S472Y, K507R, K512P, S515V, S538C, Y579W, S598Q, I602T, I602D, S605T, G609E, D676H, T694A, R698W, T699A, T711V, T711Y, K754R, S760G, T, 781M, N786K, T797S, Q834E, and S835D, wherein numbering is according to SEQ ID NO:2.

In another aspect, the variant comprises or consists of an alteration at a position corresponding to position selected from the group consisting of alterations in positions: 4, 17, 18, 20, 51, 53, 55, 56, 60, 63, 71, 79, 87, 92, 99, 120, 125, 126, 130, 137, 182, 186, 189, 192, 213, 216, 221, 226, 228, 230, 231, 232, 233, 235, 240, 243, 247, 249, 278, 279, 281, 283, 285, 289, 292, 294, 298, 302, 311, 313, 333, 346, 353, 358, 386, 387, 388, 390, 403, 408, 410, 416, 441, 448, 451, 471, 472, 476, 489, 507, 512, 515, 538, 555, 556, 557, 558, 559, 560, 561, 562, 563, 564, 567, 568, 570, 575, 578, 579, 580, 581, 583, 589, 590, 591, 592, 593, 595, 598, 599, 602, 603, 605, 607, 609, 616, 627, 630, 631, 635, 636, 638, 639, 640, 641, 642, 643, 644, 651, 676, 683, 688, 690, 694, 698, 699, 706, 711, 713, 1719, 720, 744, 749, 754, 756, 760, , 781, 786, 797, 810, 811, 812, 815, 823, 824, 825, 827, 828, 833, 834, 835, 837, 843, 848, 868, 869, 870, 871, 872, 873, 874, 880, 881, 883, 884, 885, 887, 888, 890, 892, 894, 898, 905, 906, 912, 920, 921, 924, 926, 927, 928, 932, 933, 934, 935, 937, 938, 939, 940, 941, 942, 943, 946, 948, 950, 952, 953, 954, 956, 957, 960, 966, 971, 972, 980, 989, 991, 994, 995, 998, 999, 1006, 1009, 1010, 1011, 1029, 1030, 1031, 1032, 1035, 1037, 1038, 1040, 1041, 1042, 1044, 1045, 1048 of SEQ ID NO:2.

In another aspect, the amino acid at a position corresponding to any of positions as described above is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution selected from the group consisting of: N285G, W333L, T353D, N558NP, N558F, T633V, D635L, D635M, D635T, F638Y, T639D, G994N, and K281T, G563E, 1575M, 1575A, K921D, N558K+A559K+S560F+T561P+G562W, N558K, A559K, S560F, T561P, G562W and I125V, A126R, K130R, K213R, A221R, K228E, K2281, G230F, G230L, G230A, G230H, G230N, G230W, G230T, F231Y, F231N, V232R, V232G, H235D, N240Q, G243K, G243R, A249N, A278S, K281F, K281V, K281Y, K281H, K281Q, K281N, K281W, N285L, N285M, N285S, N285P, N285T, N285Y, N285H, N285K, N285D, N285W, N285R, T292F, T292L, T2921, T292V, T292S, T292P, T292Y, T292Q, T292N, T292K, T292D, T292G, F297L, A346H, G556S, N558D, N558M, N558Q, N5581, N558Y, N558H, A559N, A559F, A559M, A559P, A559Y, A559H, A559Q, A559D, A559R, A559G, A559I, A559S, S560P, S560K, S560G, S560D, T561P, T561E, T561Q, T561S, T561D, A5641, A564Y, A564H, A564Q, A564K, A564E, E565M, V567F, K568R, L569F, L569Y, L569D, L569E, P570F, P570L, P5701, P570M, P570V, P570S, P570T, P570A, P570Y, P570H, P570Q, P570N, P570K, P570E, P570W, P570R, P570G, I575D, I575E, I576F, I576M, I576P, D578R, Y579F, Y579W, V580L, D583M, Q589G, P590S, P590T, P590E, E591L, G592D, S593P, S593H, S593Q, S593N, S593K, S593D, S593E, S593R, S616D, K627L, K627M, K627V, K627S, K627T, K627Q, K627R, I630F, I630V, I630Y, D635A, D635P, D635N, D635K, D635E, D635G, D635W, S636L, S636M, S636A, S636H, S636Q, S636N, S636K, S636R, F638I, F638V, F638T, F638L, F638H, T639V, T639S, T639L, T639I, T639M, T639A, T639E, T639W, T639G, Y641E, S642T, S642N, N643D, N643H, N643T, T644F, A651P, S810R, A811S, V812F, V812I, V812M, V812W, V812R, N815V, N815Y, N815E, N815W, N815R, S823Q, A824T, T825N, T825W, T825A, T825D, V827I, V827M, V827S, T843V, D870F, D870L, D870I, D870M, D870V, D870S, D870T, D870Y, D870H, D870Q, D870N, D870K, D870E, D870W, D870R, D870G, P871F, P871L, P871I, P871M, P871V, P871S, P871T, P871A, P871Y, P871H, P871Q, T872S, T872F, T872A, T872Y, T872H, T872Q, T872N, T872K, T872D, T872E, T872W, T872R, T872G, D873K, D873E, T874V, T874S, T874P, T874A, T874H, T874Q, T874N, T874K, V881Q, T883K, Y884H, A885F, A885Q, A885N, T887L, T8871, T887S, T887H, T887R, K894E, N920D, K921R, K921E, T932A, N933V, N933S, Y934G, Y934M, Y934S, Y934A, Y934Q, Y934N, Y934E, Y934W, Y934R, T935W, A937F, A937V, A937S, A937T, A937Q, A937D, A937E, V938I, K939I, K939V, D940E, N941S, N941H, N941D, A942P, A942E, D943Y, D943H, R950V, R950H, R950N, F952S, F952W, N953Y, G954L, Y960F, A964N, A964C, N966P, N966C, G971A, Q974K, Q974C, Q9891, Q991L, Q991I, Q991M, Q991V, Q991T, Q991K, Q991C, S995I, S995V, S995Q, S995R, S995C, G998V, G998A, 51006T, 51006A, S1006K, 51006R, Y1010W, L1011M, L1011S, L1011A, L1011Q, L1011N, L1011D, L1011E, R1029N, F1030M, K1031I, K1031S, K1031T, K1031H, V1032G, K1035A, A1037E, A1037W, 51038L, S10381, L1040N, L1040E, G1041F, L1044F, L1044S, L1044N, L1044W, P1045Q, P1045W, and A559N+Y579F, A559N, Y579F, A564E+Y579F, A564E, Y579F, A559N+Y579W, A559N, Y579W, G562P+Y579W, G562P, Y579W, A564D+Y579W, A564D, Y579W, A559N+Y579W+K99R, A559N, Y579W, K99R, A559N+Y579W+K281R, A559N, Y579W, K281R, K281R+A559N+Y579W, K281R, A559N, Y579W, A559N+Y579W+S616D, A559N, Y579W, S616D, A559N+Y579W+S636N, A559N, Y579W, S636N, A559N+Y579W+A651P, A559N, Y579W, A651P, A559N+Y579W+K948E, A559N, Y579W, K948E, A559N+Y579W+K1009E, A559N, Y579W, K1009E, A559N+Y579W+K627R, A559N, Y579W, K627R, Y579W+K921R, Y579W, K921R, A559N+Y579W+K921R, A559N, Y579W, K921R, K99R+Y579W, K99R, Y579W, Y579W+A651P, Y579W, A651P, Y579W+K948E, Y579W, K948E, Y579W+K1009E, Y579W, K1009E, A559N+Y579W+Y934G, A559N, Y579W, Y934G, A559N+Y579W+K921R+Y934G, A559N, Y579W, K921R, Y934G, A559N+Y579W+K627M, A559N, Y579W, K627M, A559N+Y579W+K627R+S616D, A559N, Y579W, K627R, S616D, A559N+Y579F+K627R, A559N, Y579F, K627R, A559N+Y579W+K921R+A651P, A559N, Y579W, K921R, A651P, A559N+Y579W+K921R+K627R, A559N, Y579W, K921R, K627R, A559N+Y579W+K921R+S636K, A559N, Y579W, K921R, S636K, A559N+Y579W+K921R+S616D, A559N, Y579W, K921R, S616D, A559N+Y579W+K921R+S636N, A559N, Y579W, K921R, S636N, A559N+Y579W+K921R+K627R+S636N, A559N, Y579W, K921R, K627R, S636N, A559N+Y579W+S636N+A651P, A559N, Y579W, S636N, A651P, A559N+Y579W+S616D+A651P, A559N, Y579W, S616D, A651P, A559N+Y579W+S616D+S636K, A559N, Y579W, S616D, S636K, A559N+Y579W+S616D+K921R+Y934G, A559N, Y579W, S616D, K921R, Y934G, A559N+Y579W+A651P+K627M, A559N, Y579W, A651P, K627M, A559N+Y579W+A651P+S636K, A559N, Y579W, A651P, S636K, A559N+Y579W+A651P+K627R+S636N, A559N, Y579W, A651P, K627R, S636N, A559N+Y579W+A651P+S616D, A559N, Y579W, A651P, S616D, A559N+Y579W+A651P+K921R+Y934G, A559N, Y579W, A651P, K921R, Y934G, S636N+Y934G, S636N, Y934G, S636N+K921R, S636N, K921R, S636N+K627R, S636N, K627R, S636N+Y579W, S636N, Y579W, F6381+Y934G, F6381, Y934G, F638I+K921R, F638I, K921R, F638I+K627R, F638I, K627R, F638I+Y579W, F638I, Y579W, K627R+K51Q, K627R, K51Q, K627R+K451S, K627R, K451S, K627R+A559N, K627R, A559N, K627R+Y579W, K627R, Y579W, Y579W+Y934G, Y579W, Y934G, A651P+F638I, A651P, F638I, P570Q+A651P, P570Q, A651P, P570Q+K921R, P570Q, K921R, P570Q+K627R, P570Q, K627R, P570Q+A559N, P570Q, A559N, P570Q+Y579W, P570Q, Y579W, P570Q+F638I, P570Q, F638I, P570K+Y579W, P570K, Y579W, P570K+F638I, P570K, F638I, P570T+A651P, P570T, A651P, P570T+S636N, P570T, S636N, P570T+Y934G, P570T, Y934G, P570T+F638I, P570T, F638I, P570T+K921R, P570T, K921R, P570T+K627R, P570T, K627R, P570T+A559N, P570T, A559N, P570T+A885F, P570T, A885F, A885F+Y934G, A885F, Y934G, A885F+K627R, A885F, K627R, A559N+Y579W+S636L, A559N, Y579W, S636L, A559N+Y579W+F638I, A559N, Y579W, F6381I, A559N+Y579W+D870M, D870M, A559N+Y579W+S560P, S560P, A559N+Y579W+A564I, A564I, A559N+Y579W+P570N, P570N, A559N+Y579W+P570K, P570K, A559N+Y579W+P570R, P570R, A559N+Y579W+P570A, P570A, A559N+Y579W+P570T, P570T, A559N+Y579W+P570S, P570S, A559N+Y579W+P570Q, P570Q, A559N+Y579W+P570H, P570H, and N558E, A559P, A559N, A559H, T561P, A564E, P570A, P570Q, P570R, P570S, P570K, P570T, P570N, Y579W, Y579F, T581M, S616D, K627R, K627M, K627Q, S636N, S636Q, S636R, S636K, S636M, 5636H, F6381, F638L, N643D, A651P, A651S, A885F, A885Q, K921R, Y934R, Y934G, N966C, L1011A, K1031I, and A559N+P570A+Y579W, A559N+P570H+Y579W, A559N+P570K+Y579W, A559N+P570N+Y579W, A559N+P570Q+Y579W, A559N+P570R+Y579W, A559N+P570S+Y579W, A559N+P570T+Y579W, A559N+S560P+Y579W, A559N+Y579W+A651P, A559N+Y579W+A651P+Y934G, A559N+Y579W+F6381, A559N+Y579W+K921R, A559N+Y579W+S616D+K921R, A559N+Y579W+S636N, A559N+Y579F, A559N+Y579W, A559N+Y579W+K921R, A559N+Y579W+S616D, F638I+Y934G, K627R+S636N, K627R+Y934G, P570K+Y579W, Q416D+A559N+Y579W+S636N, Q416D, S128X+A559N+Y579W+K627R, S128X, S128X+A559N+Y579W+S636N, Y579W+S636N, V4T, S17A, N18G, F20P, F20N, F20G, F20Y, K51Q, K51H, E53Y, E53P, E53G, Y55M, Y55D, V56M, Y60F, S63F, A71E, 579W, T87R, T92S, A120P, N129D, F137L, H182Y, A186P, N189K, K192N, N216D, N216Q, N216R, L226K, G230H, L233H, D247N, G279E, K281R, A283D, N285D, N285G, Q289E, T292A, T292F, T292Y, A294V, Q298E, I302D, I302H, I302V, I302M, H311N, S313D, A346D, A386P, I387T, K388R, K390Q, I403Y, E408D, E408N, E4085, E408P, E408A, E408G, P410G, Q4165, Q416D, N441G, A448E, A448W, A4485, K451S, K451Q, G471S, S472Y, D476R, Q489P, K507R, K512P, S515V, S538C, L555Q, G557R, N558E, A559N, A559P, A559H, A559D, S560P, S560G, T561P, A564E, A564I, V567P, K568R, P570R, P570Q, P570K, P570A, P570T, P570G, P570S, P570H, P570N, I575V, Y579W, Y579F, T581M, S593N, S593E, S595L, S598Q, A599S, I602T, I602D, V603P, S605T, S607C, G609E, S616G, S616D, K627R, K627M, K627Q, K631R, K631A, D635A, D635E, D635M, D635N, D635L, D635W, S636N, S636K, S636L, S636Q, S636R, S636M, 5636H, F638N, F638I, F638L, F638V, F638H, F638M, T639G, T639I, T639M, T639Y, T639W, T639P, T639E, T640S, S642N, S642T, N643D, N643H, A651P, A651S, D676H, Q683E, A688G, Y690F, T694A, T697G, R698W, T699A, T706Q, T711S, T711V, T711Y, K713R, W719R, K720H, K744H, K744Q, A749T, K754R, V756Y, V756H, 5760G, T, 781M, N786K, T797S, S810Q, A824D, T825G, N828D, N833D, Q834E, S835A, S835D, V8371, N848D, A868E, A869V, D870V, T872G, T872H, T872W, T872Q, R880K, V881Q, V881T, T883R, T883V, T883C, T883K, Y884H, A885N, A885Q, A885F, T887K, T887S, L888M, V890R, T892P, T892V, R898Q, N905D, F906A, Q912V, N920P, K921R, A924D, V926F, V926P, K927R, S928D, T932A, N933S, N933V, Y934G, Y934R, Y934Q, A937E, V9381, K939V, N941S, A942P, G946R, K948R, Q956Y, Q956S, A957L, A957P, N966C, T972K, M9801, G994D, T999R, L1011A, K1031I, A1037E, S1038G, G1041R, Y1042N, and F1048W, wherein numbering is according to SEQ ID NO:2.

In another aspect, a variant comprises an alteration at one or more (e.g. several) positions corresponding to positions 17, 20, 51, 53, 55, 56, 60, 63, 79, 87, 192, 302, 387, 388, 390, 403, 408, 410, 416, 448, 451, 471, 472, 507, 512, 515, 538, 598, 602, 605, 609, 676, 694, 698, 699, 711, 754, 760, , 781, 786, 797, 834, and 835 of SEQ ID NO:2.

In another aspect, a variant comprises an alteration at two positions corresponding to any of positions positions 17, 20, 51, 53, 55, 56, 60, 63, 79, 87, 192, 302, 387, 388, 390, 403, 408, 410, 416, 448, 451, 471, 472, 507, 512, 515, 538, 598, 602, 605, 609, 676, 694, 698, 699, 711, 754, 760, , 781, 786, 797, 834, and 835 of SEQ ID NO:2.

In another aspect, a variant comprises an alteration at three positions corresponding to any of positions positions 17, 20, 51, 53, 55, 56, 60, 63, 79, 87, 192, 302, 387, 388, 390, 403, 408, 410, 416, 448, 451, 471, 472, 507, 512, 515, 538, 598, 602, 605, 609, 676, 694, 698, 699, 711, 754, 760, , 781, 786, 797, 834, and 835 of SEQ ID NO:2. Thus, in one embodiment, the variant comprises an alteration in the positions corresponding to: 17+20, 17+51, 17+53, 17+55, 17+56, 17+60, 17+63, 17+79, 17+87, 17+192, 17+302, 7+387, 17+388, 17+390, 17+403, 17+408, 17+410, 17+416, 17+448, 17+451, 17+471, 17+472, 17+507, 17+512, 17+515, 17+538, 17+598, 17+602, 17+605, 17+609, 17+676, 17+694, 17+698, 17+699, 17+711, 17+754, 17+760, 17+781, 17+786, 17+797, 17+834, 17+835, 20+51, 20+53, 20+55, 20+56, 20+60, 20+63, 20+79, 20+87, 20+192, 20+302, 20+387, 20+388, 20+390, 20+403, 20+408, 20+410, 20+416, 20+448, 20+451, 20+471, 20+472, 20+507, 20+512, 20+515, 20+538, 20+598, 20+602, 20+605, 20+609, 20+676, 20+694, 20+698, 20+699, 20+711, 20+754, 20+760, 20+781, 20+786, 20+797, 20+834, 20+835, 51+53, 51+55, 51+56, 51+60, 51+63, 51+79, 51+87, 51+192, 51+302, 51+387, 51+388, 51+390, 51+403, 51+408, 51+410, 51+416, 51+448, 51+451, 51+471, 51+472, 51+507, 51+512, 51+515, 51+538, 51+598, 51+602, 51+605, 51+609, 51+676, 51+694, 51+698, 51+699, 51+711, 51+754, 51+760, 51+781, 51+786, 51+797, 51+834, 51+835, 53+55, 53+56, 53+60, 53+63, 53+79, 53+87, 53+192, 53+302, 53+387, 53+388, 53+390, 53+403, 53+408, 53+410, 53+416, 53+448, 53+451, 53+471, 53+472, 53+507, 53+512, 53+515, 53+538, 53+598, 53+602, 53+605, 53+609,3+676, 53+694, 53+698, 53+699, 53+711, 53+754, 53+760, 53+781, 53+786, 53+797, 53+834, 53+835, 55+56, 55+60, 55+63, 55+79, 55+87, 55+192, 55+302, 55+387, 55+388, 55+390, 55+403, 55+408, 55+410, 55+416, 55+448, 55+451, 55+471, 55+472, 55+507, 55+512, 55+515, 55+538, 55+598, 55+602, 55+605, 55+609, 55+676, 55+694, 55+698, 55+699, 55+711, 55+754, 55+760, 55+781, 55+786, 55+797, 55+834, 55+835, 56+60, 56+63, 56+79, 56+87, 56+192, 56+302, 56+387, 56+388, 56+390, 56+403, 56+408, 56+410, 56+416, 56+448, 56+451, 56+471, 56+472, 56+507, 56+512, 56+515, 56+538, 56+598, 56+602, 56+605, 56+609, 56+676, 56+694, 56+698, 56+699, 56+711, 56+754, 56+760, 56+781, 56+786, 56+797, 56+834, 56+835, 60+63, 60+79, 60+87, 60+192, 60+302, 60+387, 60+388, 60+390, 60+403, 60+408, 60+410, 60+416, 60+448, 60+451, 60+471, 60+472, 60+507, 60+512, 60+515, 60+538, 60+598, 60+602, 60+605, 60+609, 60+676, 60+694, 60+698, 60+699, 60+711, 60+754, 60+760, 60+781, 60+786, 60+797, 60+834, 60+835, 63+79, 63+87, 63+192, 63+302, 63+387, 63+388, 63+390, 63+403, 63+408, 63+410, 63+416, 63+448, 63+451, 63+471, 63+472, 63+507, 63+512, 63+515, 63+538, 63+598, 63+602, 63+605, 63+609, 63+676, 63+694, 63+698, 63+699, 63+711, 63+754, 63+760, 63+781, 63+786, 63+797, 63+834, 63+835, 79+87, 79+192, 79+302, 79+387, 79+388, 79+390, 79+403, 79+408, 79+410, 79+416, 79+448, 79+451, 79+471, 79+472, 79+507, 79+512, 79+515, 79+538, 79+598, 79+602, 79+605, 79+609, 79+676, 79+694, 79+698, 79+699, 79+711, 79+754, 79+760, 79+781, 79+786, 79+797, 79+834, 79+835, 87+192, 87+302, 87+387, 87+388, 87+390, 87+403, 87+408, 87+410, 87+416, 87+448, 87+451, 87+471, 87+472, 87+507, 87+512, 87+515, 87+538, 87+598, 87+602, 87+605, 87+609, 87+676, 87+694, 87+698, 87+699, 87+711, 87+754, 87+760, 87+781, 87+786, 87+797, 87+834, 87+835, 192+302, 192+387, 192+388, 192+390, 192+403, 192+408, 192+410, 192+416, 192+448, 192+451, 192+47t192+472, 192+507, 192+512, 192+515, 192+538, 192+598, 192+602, 192+605, 192+609, 192+676, 192+694, 192+698, 192+699, 192+711, 192+754, 192+760, 192+78t192+786, 192+797, 192+834, 192+835, 302+387, 302+388, 302+390, 302+403, 302+408, 302+410, 302+416, 302+448, 302+451, 302+471, 302+472, 302+507, 302+512, 302+515, 302+538, 302+598, 302+602, 302+605, 302+609, 302+676, 302+694, 302+698, 302+699, 302+711, 302+754, 302+760, 302+781, 302+786, 302+797, 302+834, 302+835, 387+388, 387+390, 387+403, 387+408, 387+410, 387+416, 387+448, 387+451, 387+471, 387+472, 387+507, 387+512, 387+515, 387+538, 387+598, 387+602, 387+605, 87+609, 387+676, 387+694, 387+698, 387+699, 387+711387+754, 387+760, 387+781, 387+786, 387+797, 387+834, 387+835, 388+390, 388+403, 388+408, 388+410, 388+416, 388+448, 388+451, 388+471, 388+472, 388+507, 388+512, 388+515, 388+538, 388+598, 388+602, 388+605, 388+609, 388+676, 388+694, 388+698, 388+699, 388+711, 388+754, 388+760, 388+781, 388+786, 388+797, 388+834, 388+835, 390+403, 390+408, 390+410, 390+416, 390+448, 390+451, 390+471, 390+472, 390+507, 390+512, 390+515, 390+538, 390+598, 390+602, 390+605, 390+609, 390+676, 390+694, 390+698, 390+699, 390+711, 390+754, 390+760, 390+781, 390+786, 390+797, 390+834, 390+835, 403+408, 403+410, 403+416, 403+448, 403+451, 403+471, 403+472, 403+507, 403+512, 403+515, 403+538, 403+598, 403+602, 403+605, 403+609, 403+676, 403+694, 403+698, 403+699, 403+711, 403+754, 403+760, 403+781, 403+786, 403+797, 403+834, 403+835, 408+410, 408+416, 408+448, 408+451, 408+471, 408+472, 408+507, 408+512, 408+515, 408+538, 408+598, 408+602, 408+605, 408+609, 408+676, 408+694, 408+698, 408+699, 408+711, 408+754, 408+760, 408+781, 408+786, 408+797, 408+834, 408+835, 410+416, 410+448, 410+451, 410+471, 410+472, 410+507, 410+512, 410+515, 410+538, 410+598, 410+602, 410+605, 410+609, 410+676, 410+694, 410+698, 410+699, 410+711, 410+754, 410+760, 410+781, 410+786, 410+797, 410+834, 410+835, 416+448, 416+451, 416+471, 416+472, 416+507, 416+512, 416+515, 416+538, 416+598, 416+602, 416+605, 416+609, 416+676, 416+694, 416+698, 416+699, 416+711, 416+754, 416+760, 416+781, 416+786, 416+797, 416+834, 416+835, 448+451, 448+471, 448+472, 448+507, 448+512, 448+515, 448+538, 448+598, 448+602, 448+605, 448+609, 448+676, 448+694, 448+698, 448+699, 448+711, 448+754, 448+760, 448+781, 448+786, 448+797, 448+834, 448+835, 451+471, 451+472, 451+507, 451+512, 451+515, 451+538, 451+598, 451+602, 451+605, 451+609, 451+676, 451+694, 451+698, 451+699, 451+711, 451+754, 451+760, 451+781, 451+786, 451+797, 451+834, 451+835, 471+472, 471+507, 471+512, 471+515, 471+538, 471+598, 471+602, 471+605, 471+609, 471+676, 471+694, 471+698, 471+699, 471+711, 471+754, 471+760, 471+781, 471+786, 471+797, 471+834, 471+835, 472+507, 472+512, 472+515, 472+538, 472+598, 472+602, 472+605, 472+609, 472+676, 472+694, 472+698, 472+699, 472+711, 472+754, 472+760, 472+781, 472+786, 472+797, 472+834, 472+835, 507+512, 507+515, 507+538, 507+598, 507+602, 507+605, 507+609, 507+676, 507+694, 507+698, 507+699, 507+711, 507+754, 507+760, 507+781, 507+786, 507+797, 507+834, 507+835, 512+515, 512+538, 512+598, 512+602, 512+605, 512+609, 512+676, 512+694, 512+698, 512+699, 512+711, 512+754, 512+760, 512+781, 512+786, 512+797, 512+834, 512+835, 515+538, 515+598, 515+602, 515+605, 515+609, 515+676, 515+694, 515+698, 515+699, 515+711, 515+754, 515+760, 515+781, 515+786, 515+797, 515+834, 515+835, 538+598, 538+602, 538+605, 538+609, 538+676, 538+694, 538+698, 538+699, 538+711, 538+754, 538+760, 538+781, 538+786, 538+797, 538+834, 538+835, 598+602, 598+605, 598+609, 598+676, 598+694, 598+698, 598+699, 598+711, 598+754, 598+760, 598+781, 598+786, 598+797, 598+834, 598+835, 602+605, 602+609, 602+676, 602+694, 602+698, 602+699, 602+711, 602+754, 602+760, 602+781, 602+786, 602+797, 602+834, 602+835, 605+609, 605+676, 605+694, 605+698, 605+699, 605+711, 605+754, 605+760, 605+781, 605+786, 605+797, 605+834, 605+835, 609+676, 609+694, 609+698, 609+699, 609+711, 609+754, 609+760, 609+781, 609+786, 609+797, 609+834, 609+835, 676+694, 676+698, 676+699, 676+711, 676+754, 676+760, 676+781, 676+786, 676+797, 676+834, 676+835, 694+698, 694+699, 694+711, 694+754, 694+760, 694+781, 694+786, 694+797, 694+834, 694+835, 698+699, 698+711, 698+754, 698+760, 698+781, 698+786, 698+797, 698+834, 698+835, 699+711, 699+754, 699+760, 699+781, 699+786, 699+797, 699+834, 699+835, 711+754, 711+760, 711+781, 711+786, 711+797, 711+834, 711+835, 754+760, 754+781, 754+786, 754+797, 754+834, 754+835 760+781, 760+786, 760+797, 760+834, 760+835, , 781+786, , 781+797, , 781+834, , 781+835, 786+797, 786+834, 786+835, 797+834, 797+835, 834+835, 17+20+51, 17+20+53, 17+20+55, 17+20+56, 17+20+60, 17+20+63, 17+20+79, 17+20+87, 17+20+192, 17+20+302, 17+20+387, 17+20+388, 17+20+390, 17+20+403, 17+20+408, 17+20+410, 17+20+416, 17+20+448, 17+20+451, 17+20+471, 7+20+472, 17+20+507, 17+20+512, 17+20+515, 17+20+538, 17+20+598, 17+20+602, 17+20+605, 17+20+609, 17+20+676, 17+20+694, 17+20+698, 17+20+699, 17+20+711, 17+20+754, 17+20+760, 17+20+781, 17+20+786, 17+20+797, 17+20+834, 17+20+835, 17+51+53, 17+51+55, 17+51+56, 17+51+60, 17+51+63, 17+51+79, 17+51+87, 17+51+192, 17+51+302, 17+51+387, 17+51+388, 17+51+390, 17+51+403, 17+51+408, 17+51+410, 17+51+416, 17+51+448, 17+51+451, 17+51+471, 17+51+472, 17+51+507, 17+51+512, 17+51+515, 17+51+538, 17+51+598, 17+51+602, 17+51+605, 17+51+609, 17+51+676, 17+51+694, 17+51+698, 17+51+699, 17+51+711, 17+51+754, 17+51+760, 17+51+781, 17+51+786, 17+51+797, 17+51+834, 17+51+835, 17+53+55, 17+53+56, 17+53+60, 17+53+63, 17+53+79, 17+53+87, 17+53+192, 17+53+302, 17+53+387, 17+53+388, 17+53+390, 17+53+403, 17+53+408, 17+53+410, 17+53+416, 17+53+448, 17+53+451, 17+53+471, 17+53+472, 17+53+507, 17+53+512, 17+53+515, 17+53+538, 17+53+598, 17+53+602, 17+53+605, 17+53+609, 17+53+676, 17+53+694, 17+53+698, 17+53+699, 17+53+711, 17+53+754, 17+53+760, 17+53+781, 17+53+786, 17+53+797, 17+53+834, 17+53+835, 17+55+56, 17+55+60, 17+55+63, 17+55+79, 17+55+87, 17+55+192, 17+55+302, 17+55+387, 17+55+388, 17+55+390, 17+55+403, 17+55+408, 17+55+410, 17+55+416, 17+55+448, 17+55+451, 17+55+471, 17+55+472, 17+55+507, 17+55+512, 17+55+515, 17+55+538, 17+55+598, 17+55+602, 17+55+605, 17+55+609, 17+55+676, 17+55+694, 17+55+698, 17+55+699, 17+55+711, 17+55+754, 17+55+760, 17+55+781, 17+55+786, 17+55+797, 17+55+834, 17+55+835, 17+56+60, 17+56+63, 17+56+79, 17+56+87, 17+56+192, 17+56+302, 17+56+387, 17+56+388, 17+56+390, 17+56+403, 17+56+408, 17+56+410, 17+56+416, 17+56+448, 17+56+451, 17+56+471, 17+56+472, 17+56+507, 17+56+512, 17+56+515, 17+56+538, 17+56+598, 17+56+602, 17+56+605, 17+56+609, 17+56+676, 17+56+694, 17+56+698, 17+56+699, 17+56+711, 17+56+754, 17+56+760, 17+56+781, 17+56+786, 17+56+797, 17+56+834, 17+56+835, 17+60+63, 17+60+79, 17+60+87, 17+60+192, 17+60+302, 17+60+387, 17+60+388, 17+60+390, 17+60+403, 17+60+408, 17+60+410, 17+60+416, 17+60+448, 17+60+451, 17+60+471, 17+60+472, 17+60+507, 17+60+512, 17+60+515, 17+60+538, 17+60+598, 17+60+602, 17+60+605, 17+60+609, 17+60+676, 17+60+694, 17+60+698, 17+60+699, 17+60+711, 17+60+754, 17+60+760, 17+60+781, 17+60+786, 17+60+797, 17+60+834, 17+60+835, 17+63+79, 17+63+87, 17+63+192, 17+63+302, 17+63+387, 17+63+388, 17+63+390, 17+63+403, 17+63+408, 17+63+410, 17+63+416, 17+63+448, 17+63+451, 17+63+471, 17+63+472, 17+63+507, 17+63+512, 17+63+515, 17+63+538, 17+63+598, 17+63+602, 17+63+605, 17+63+609, 17+63+676, 17+63+694, 17+63+698, 17+63+699, 17+63+711, 17+63+754, 17+63+760, 17+63+781, 17+63+786, 17+63+797, 17+63+834, 17+63+835, 17+79+87, 17+79+192, 17+79+302, 17+79+387, 17+79+388, 17+79+390, 17+79+403, 17+79+408, 17+79+410, 17+79+416, 17+79+448, 17+79+451, 17+79+471, 17+79+472, 17+79+507, 17+79+512, 17+79+515, 17+79+538, 17+79+598, 17+79+602, 17+79+605, 17+79+609, 17+79+676, 17+79+694, 17+79+698, 17+79+699, 17+79+711, 17+79+754, 17+79+760, 17+79+781, 17+79+786, 17+79+797, 17+79+834, 17+79+835, 17+87+192, 17+87+302, 17+87+387, 17+87+388, 17+87+390, 17+87+403, 17+87+408, 17+87+410, 17+87+416, 17+87+448, 17+87+451, 17+87+471, 17+87+472, 17+87+507, 17+87+512, 17+87+515, 17+87+538, 17+87+598, 17+87+602, 17+87+605, 17+87+609, 17+87+676, 17+87+694, 17+87+698, 17+87+699, 17+87+711, 17+87+754, 17+87+760, 17+87+781, 17+87+786, 17+87+797, 17+87+834, 17+87+835, 17+192+302, 17+192+387, 17+192+388, 17+192+390, 17+192+403, 17+192+408, 17+192+410, 17+192+416, 17+192+448, 17+192+451, 17+192+471, 17+192+472, 17+192+507, 17+192+512, 17+192+515, 17+192+538, 17+192+598, 17+192+602, 17+192+605, 17+192+609, 17+192+676, 17+192+694, 17+192+698, 17+192+699, 17+192+711, 17+192+754, 17+192+760, 17+192+781, 17+192+786, 17+192+797, 17+192+834, 17+192+835, 17+302+387, 17+302+388, 17+302+390, 17+302+403, 17+302+408, 17+302+410, 17+302+416, 17+302+448, 17+302+451, 17+302+471, 17+302+472, 17+302+507, 17+302+512, 17+302+515, 17+302+538, 17+302+598, 17+302+602, 17+302+605, 17+302+609, 17+302+676, 17+302+694, 17+302+698, 17+302+699, 17+302+711, 17+302+754, 17+302+760, 17+302+781, 17+302+786, 17+302+797, 17+302+834, 17+302+835, 17+387+388, 17+387+390, 17+387+403, 17+387+408, 17+387+410, 17+387+416, 17+387+448, 17+387+451, 17+387+471, 17+387+472, 17+387+507, 17+387+512, 17+387+515, 17+387+538, 17+387+598, 17+387+602, 17+387+605, 17+387+609, 17+387+676, 17+387+694, 17+387+698, 17+387+699, 17+387+711, 17+387+754, 17+387+760, 17+387+781, 17+387+786, 17+387+797, 17+387+834, 17+387+835, 17+388+390, 17+388+403, 17+388+408, 17+388+410, 17+388+416, 17+388+448, 17+388+451, 17+388+471, 17+388+472, 17+388+507, 17+388+512, 17+388+515, 17+388+538, 17+388+598, 17+388+602, 17+388+605, 17+388+609, 17+388+676, 17+388+694, 17+388+698, 17+388+699, 17+388+711, 17+388+754, 17+388+760, 17+388+781, 17+388+786, 17+388+797, 17+388+834, 17+388+835, 17+390+403, 17+390+408, 17+390+410, 17+390+416, 17+390+448, 17+390+451, 17+390+471, 17+390+472, 17+390+507, 17+390+512, 17+390+515, 17+390+538, 17+390+598, 17+390+602, 17+390+605, 17+390+609, 17+390+676, 17+390+694, 17+390+698, 17+390+699, 17+390+711, 17+390+754, 17+390+760, 17+390+781, 17+390+786, 17+390+797, 17+390+834, 17+390+835, 17+403+408, 17+403+410, 17+403+416, 17+403+448, 17+403+451, 17+403+471, 17+403+472, 17+403+507, 17+403+512, 17+403+515, 17+403+538, 17+403+598, 17+403+602, 17+403+605, 17+403+609, 17+403+676, 17+403+694, 17+403+698, 17+403+699, 17+403+711, 17+403+754, 17+403+760, 17+403+781, 17+403+786, 17+403+797, 17+403+834, 17+403+835, 17+408+410, 17+408+416, 17+408+448, 17+408+451, 17+408+471, 17+408+472, 17+408+507, 17+408+512, 17+408+515, 17+408+538, 17+408+598, 17+408+602, 17+408+605, 17+408+609, 17+408+676, 17+408+694, 17+408+698, 17+408+699, 17+408+711, 17+408+754, 17+408+760, 17+408+781, 17+408+786, 17+408+797, 17+408+834, 17+408+835, 17+410+416, 17+410+448, 17+410+451, 17+410+471, 17+410+472, 17+410+507, 17+410+512, 17+410+515, 17+410+538, 17+410+598, 17+410+602, 17+410+605, 17+410+609, 17+410+676, 17+410+694, 17+410+698, 17+410+699, 17+410+711, 17+410+754, 17+410+760, 17+410+781, 17+410+786, 17+410+797, 17+410+834, 17+410+835, 17+416+448, 17+416+451, 17+416+471, 17+416+472, 17+416+507, 17+416+512, 17+416+515, 17+416+538, 17+416+598, 17+416+602, 17+416+605, 17+416+609, 17+416+676, 17+416+694, 17+416+698, 17+416+699, 7+416+711, 17+416+754, 17+416+760, 17+416+781, 17+416+786, 17+416+797, 17+416+834, 17+416+835, 17+448+451, 17+448+471, 17+448+472, 17+448+507, 17+448+512, 17+448+515, 17+448+538, 17+448+598, 17+448+602, 17+448+605, 17+448+609, 17+448+676, 17+448+694, 17+448+698, 17+448+699, 17+448+711, 17+448+754, 17+448+760, 17+448+781, 17+448+786, 17+448+797, 17+448+834, 17+448+835, 17+451+471, 17+451+472, 17+451+507, 17+451+512, 17+451+515, 17+451+538, 17+451+598, 17+451+602, 17+451+605, 17+451+609, 17+451+676, 17+451+694, 17+451+698, 17+451+699, 17+451+711, 17+451+754, 17+451+760, 17+451+781, 17+451+786, 17+451+797, 17+451+834, 17+451+835, 17+471+472, 17+471+507, 17+471+512, 17+471+515, 17+471+538, 17+471+598, 17+471+602, 17+471+605, 17+471+609, 17+471+676, 17+471+694, 17+471+698, 17+471+699, 17+471+711, 17+471+754, 17+471+760, 17+471+781, 17+471+786, 17+471+797, 17+471+834, 17+471+835, 17+472+507, 17+472+512, 17+472+515, 17+472+538, 17+472+598, 17+472+602, 17+472+605, 17+472+609, 17+472+676, 17+472+694, 17+472+698, 17+472+699, 17+472+711, 17+472+754, 17+472+760, 17+472+781, 17+472+786, 17+472+797, 17+472+834, 17+472+835, 17+507+512, 17+507+515, 17+507+538, 17+507+598, 17+507+602, 17+507+605, 17+507+609, 17+507+676, 17+507+694, 17+507+698, 17+507+699, 17+507+711, 17+507+754, 17+507+760, 17+507+781, 17+507+786, 17+507+797, 17+507+834, 17+507+835, 17+512+515, 17+512+538, 17+512+598, 17+512+602, 17+512+605, 17+512+609, 17+512+676, 17+512+694, 17+512+698, 17+512+699, 17+512+711, 17+512+754, 17+512+760, 17+512+781, 17+512+786, 17+512+797, 17+512+834, 17+512+835, 17+515+538, 17+515+598, 17+515+602, 17+515+605, 17+515+609, 17+515+676, 17+515+694, 17+515+698, 17+515+699, 17+515+711, 17+515+754, 17+515+760, 17+515+781, 17+515+786, 17+515+797, 17+515+834, 17+515+835, 17+538+598, 17+538+602, 17+538+605, 17+538+609, 17+538+676, 17+538+694, 17+538+698, 17+538+699, 17+538+711, 17+538+754, 17+538+760, 17+538+781, 17+538+786, 17+538+797, 17+538+834, 17+538+835, 17+598+602, 17+598+605, 17+598+609, 17+598+676, 17+598+694, 17+598+698, 17+598+699, 17+598+711, 17+598+754, 17+598+760, 17+598+781, 17+598+786, 17+598+797, 17+598+834, 17+598+835, 17+602+605, 17+602+609, 17+602+676, 17+602+694, 17+602+698, 17+602+699, 17+602+711, 17+602+754, 17+602+760, 17+602+781, 17+602+786, 17+602+797, 17+602+834, 17+602+835, 17+605+609, 17+605+676, 17+605+694, 17+605+698, 17+605+699, 17+605+711, 17+605+754, 17+605+760, 17+605+78t17+605+786, 17+605+797, 17+605+834, 17+605+835, 17+609+676, 17+609+694, 17+609+698, 17+609+699, 17+609+711, 17+609+754, 17+609+760, 17+609+781, 17+609+786, 17+609+797, 17+609+834, 17+609+835, 17+676+694, 17+676+698, 17+676+699, 17+676+711, 17+676+754, 17+676+760, 17+676+781, 17+676+786, 17+676+797, 17+676+834, 17+676+835, 17+694+698, 17+694+699, 17+694+711, 17+694+754, 17+694+760, 17+694+781, 17+694+786, 17+694+797, 17+694+834, 17+694+835, 17+698+699, 17+698+711, 17+698+754, 17+698+760, 17+698+78t17+698+786, 17+698+797, 17+698+834, 17+698+835, 17+699+711, 17+699+754, 17+699+760, 17+699+781, 17+699+786, 17+699+797, 17+699+834, 17+699+835, 17+711+754, 17+711+760, 17+711+781, 17+711+786, 17+711+797, 17+711+834, 17+711+835, 17+754+760, 17+754+78t17+754+786, 17+754+797, 17+754+834, 17+754+835, 17+760+781, 17+760+786, 17+760+797, 17+760+834, 17+760+835, 17+781+786, 17+781+797, 17+781+834, 17+781+835, 17+786+797, 17+786+834, 17+786+835, 17+797+834, 17+797+835, 17+834+835, 20+51+53, 20+51+55, 20+51+56, 20+51+60, 20+51+63, 20+51+79, 20+51+87, 20+51+192, 20+51+302, 20+51+387, 20+51+388, 20+51+390, 20+51+403, 20+51+408, 20+51+410, 20+51+416, 20+51+448, 20+51+451, 20+51+471, 20+51+472, 20+51+507, 20+51+51220+51+515, 20+51+538, 20+51+598, 20+51+602, 20+51+605, 20+51+609, 20+51+676, 20+51+694, 20+51+698, 20+51+699, 20+51+711, 20+51+754, 20+51+760, 20+51+781, 20+51+786, 20+51+797, 20+51+834, 20+51+835, 20+53+55, 20+53+56, 20+53+60, 20+53+63, 20+53+79, 20+53+87, 20+53+192, 20+53+302, 20+53+387, 20+53+388, 20+53+390, 20+53+403, 20+53+408, 20+53+410, 20+53+416, 20+53+448, 20+53+451, 20+53+471, 20+53+472, 20+53+507, 20+53+512, 20+53+515, 20+53+538, 20+53+598, 20+53+602, 20+53+605, 20+53+609, 20+53+676, 20+53+694, 20+53+698, 20+53+699, 20+53+711, 20+53+754, 20+53+760, 20+53+781, 20+53+786, 20+53+797, 20+53+834, 20+53+835, 20+55+56, 20+55+60, 20+55+63, 20+55+79, 20+55+87, 20+55+192, 20+55+302, 20+55+387, 20+55+388, 20+55+390, 20+55+403, 20+55+408, 20+55+410, 20+55+416, 20+55+448, 20+55+45120+55+471, 20+55+472, 20+55+507, 20+55+512, 20+55+515, 20+55+538, 20+55+598, 20+55+602, 20+55+605, 20+55+609, 20+55+676, 20+55+694, 20+55+698, 20+55+699, 20+55+711, 20+55+754, 20+55+760, 20+55+781, 20+55+786, 20+55+797, 20+55+834, 20+55+835, 20+56+60, 20+56+63, 20+56+79, 20+56+87, 20+56+192, 20+56+302, 20+56+387, 20+56+388, 20+56+390, 20+56+403, 20+56+408, 20+56+410, 20+56+416, 20+56+448, 20+56+451, 20+56+471, 20+56+472, 20+56+507, 20+56+512, 20+56+515, 20+56+538, 20+56+598, 20+56+602, 20+56+605, 20+56+609, 20+56+676, 20+56+694, 20+56+698, 20+56+699, 20+56+711, 20+56+754, 20+56+760, 20+56+781, 20+56+786, 20+56+797, 20+56+834, 20+56+835, 20+60+63, 20+60+79, 20+60+87, 20+60+192, 20+60+302, 20+60+387, 20+60+388, 20+60+390, 20+60+403, 20+60+408, 20+60+410, 20+60+416, 20+60+448, 20+60+451, 20+60+471, 20+60+472, 20+60+507, 20+60+512, 20+60+515, 20+60+538, 20+60+598, 20+60+602, 20+60+605, 20+60+609, 20+60+676, 20+60+694, 20+60+698, 20+60+699, 20+60+711, 20+60+754, 20+60+760, 20+60+781, 20+60+786, 20+60+797, 20+60+834, 20+60+835, 20+63+79, 20+63+87, 20+63+192, 20+63+302, 20+63+387, 20+63+388, 20+63+390, 20+63+403, 20+63+408, 20+63+410, 20+63+416, 20+63+448, 20+63+451, 20+63+471, 20+63+472, 20+63+507, 20+63+512, 20+63+515, 20+63+538, 20+63+598, 20+63+602, 20+63+605, 20+63+609, 20+63+676, 20+63+694, 20+63+698, 20+63+699, 20+63+711, 20+63+754, 20+63+760, 20+63+781, 20+63+786, 20+63+797, 20+63+834, 20+63+835, 20+79+87, 20+79+192, 20+79+302, 20+79+387, 20+79+388, 20+79+390, 20+79+403, 20+79+408, 20+79+410, 20+79+416, 20+79+448, 20+79+451, 20+79+471, 20+79+472, 20+79+507, 20+79+512, 20+79+515, 20+79+538, 20+79+598, 20+79+602, 20+79+605, 20+79+609, 20+79+676, 20+79+694, 20+79+698, 20+79+699, 20+79+711, 20+79+754, 20+79+760, 20+79+781, 20+79+786, 20+79+797, 20+79+834, 20+79+835, 20+87+192, 20+87+302, 20+87+387, 20+87+388, 20+87+390, 20+87+403, 20+87+408, 20+87+410, 20+87+416, 20+87+448, 20+87+451, 20+87+471, 20+87+472, 20+87+507, 20+87+512, 20+87+515, 20+87+538, 20+87+598, 20+87+602, 20+87+605, 20+87+609, 20+87+676, 20+87+694, 20+87+698, 20+87+699, 20+87+711, 20+87+754, 20+87+760, 20+87+781, 20+87+786, 20+87+797, 20+87+834, 20+87+835, 20+192+302, 20+192+387, 20+192+388, 20+192+390, 20+192+403, 20+192+408, 20+192+410, 20+192+416, 20+192+448, 20+192+451, 20+192+471, 20+192+472, 20+192+507, 20+192+512, 20+192+515, 20+192+538, 20+192+598, 20+192+602, 20+192+605, 20+192+609, 20+192+676, 20+192+694, 20+192+698, 20+192+699 20+192+711, 20+192+754, 20+192+760, 20+192+781, 20+192+786, 20+192+797, 20+192+834, 20+192+835, 20+302+387, 20+302+388, 20+302+390, 20+302+403, 20+302+408, 20+302+410, 20+302+416, 20+302+448, 20+302+451, 20+302+471, 20+302+472, 20+302+507, 20+302+512, 20+302+515, 20+302+538, 20+302+598, 20+302+602, 20+302+605, 20+302+609, 20+302+676, 20+302+694, 20+302+698, 20+302+699, 20+302+711, 20+302+754, 20+302+760, 20+302+781, 20+302+786, 20+302+797, 20+302+834, 20+302+835, 20+387+388, 20+387+390, 20+387+403, 20+387+408, 20+387+410, 20+387+416, 20+387+448, 20+387+451, 20+387+471, 20+387+472, 20+387+507, 20+387+512, 20+387+515, 20+387+538, 20+387+598, 20+387+602, 20+387+605, 20+387+609, 20+387+676, 20+387+694, 20+387+698, 20+387+699, 20+387+711, 20+387+754, 20+387+760, 20+387+781, 20+387+786, 20+387+797, 20+387+834, 20+387+835, 20+388+390, 20+388+403, 20+388+408, 20+388+410, 20+388+416, 20+388+448, 20+388+451, 20+388+471, 20+388+472, 20+388+507, 20+388+512, 20+388+515, 20+388+538, 20+388+598, 20+388+602, 20+388+605, 20+388+609, 20+388+676, 20+388+694, 20+388+698, 20+388+699, 20+388+711, 20+388+754, 20+388+760, 20+388+781, 20+388+786, 20+388+797, 20+388+834, 20+388+835, 20+390+403, 20+390+408, 20+390+410, 20+390+416, 20+390+448, 20+390+451, 20+390+471, 20+390+472, 20+390+507, 20+390+512, 20+390+515, 20+390+538, 20+390+598, 20+390+602, 20+390+605, 20+390+609, 20+390+676, 20+390+694, 20+390+698, 20+390+699, 20+390+711, 20+390+754, 20+390+760, 20+390+781, 20+390+786, 20+390+797, 20+390+834, 20+390+835, 20+403+408, 20+403+410, 20+403+416, 20+403+448, 20+403+451, 20+403+471, 20+403+472, 20+403+507, 20+403+512, 20+403+515, 20+403+538, 20+403+598, 20+403+602, 20+403+605, 20+403+609, 20+403+676, 20+403+694, 20+403+698, 20+403+699, 20+403+711, 20+403+754, 20+403+760, 20+403+781, 20+403+786, 20+403+797, 20+403+834, 20+403+835, 20+408+410, 20+408+416, 20+408+448, 20+408+451, 20+408+471, 20+408+472, 20+408+507, 20+408+512, 20+408+515, 20+408+538, 20+408+598, 20+408+602, 20+408+605, 20+408+609, 20+408+676, 20+408+694, 20+408+698, 20+408+699, 20+408+711, 20+408+754, 20+408+760, 20+408+781, 20+408+786, 20+408+797, 20+408+834, 20+408+835, 20+410+416, 20+410+448, 20+410+451, 20+410+471, 20+410+472, 20+410+507, 20+410+512, 20+410+515, 20+410+538, 20+410+598, 20+410+602, 20+410+605, 20+410+609, 20+410+676, 20+410+694, 20+410+698, 20+410+699, 20+410+711, 20+410+754, 20+410+760, 20+410+781, 20+410+786, 20+410+797, 20+410+834, 20+410+835, 20+416+448, 20+416+451, 20+416+471, 20+416+472, 20+416+507, 20+416+512, 20+416+515, 20+416+538, 20+416+598, 20+416+602, 20+416+605, 20+416+609, 20+416+676, 20+416+694, 20+416+698, 20+416+699, 20+416+711, 20+416+754, 20+416+760, 20+416+781, 20+416+786, 20+416+797, 20+416+834, 20+416+835, 20+448+451, 20+448+471, 20+448+472, 20+448+507, 20+448+512, 20+448+515, 20+448+538, 20+448+598, 20+448+602, 20+448+605, 20+448+609, 20+448+676, 20+448+694, 20+448+698, 20+448+699, 20+448+711, 20+448+754, 20+448+760, 20+448+781, 20+448+786, 20+448+797, 20+448+834, 20+448+835, 20+451+471, 20+451+472, 20+451+507, 20+451+512, 20+451+515, 20+451+538, 20+451+598, 20+451+602, 20+451+605, 20+451+609, 20+451+676, 20+451+694, 20+451+698, 20+451+699, 20+451+711, 20+451+754, 20+451+760, 20+451+781, 20+451+786, 20+451+797, 20+451+834, 20+451+835, 20+471+472, 20+471+507, 20+471+512, 20+471+515, 20+471+538, 20+471+598, 20+471+602, 20+471+605, 20+471+609, 20+471+676, 20+471+694, 20+471+698, 20+471+699, 20+471+711, 20+471+754, 20+471+760, 20+471+781, 20+471+786, 20+471+797, 20+471+834, 20+471+835, 20+472+507, 20+472+512, 20+472+515, 20+472+538, 20+472+598, 20+472+602, 20+472+605, 20+472+609, 20+472+676, 20+472+694, 20+472+698, 20+472+699, 20+472+711, 20+472+754, 20+472+760, 20+472+781, 20+472+786, 20+472+797, 20+472+834, 20+472+835, 20+507+512, 20+507+515, 20+507+538, 20+507+598, 20+507+602, 20+507+605, 20+507+609, 20+507+676, 20+507+694, 20+507+698, 20+507+699, 20+507+711, 20+507+754, 20+507+760, 20+507+781, 20+507+786, 20+507+797, 20+507+834, 20+507+835, 20+512+515, 20+512+538, 20+512+598, 20+512+602, 20+512+605, 20+512+609, 20+512+676, 20+512+694, 20+512+698, 20+512+699, 20+512+711, 20+512+754, 20+512+760, 20+512+781, 20+512+786, 20+512+797, 20+512+834, 20+512+835, 20+515+538, 20+515+598, 20+515+602, 20+515+605, 20+515+609, 20+515+676, 20+515+694, 20+515+698, 20+515+699, 20+515+711, 20+515+754, 20+515+760, 20+515+781, 20+515+786, 20+515+797, 20+515+834, 20+515+835, 20+538+598, 20+538+602, 20+538+605, 20+538+609, 20+538+676, 20+538+694, 20+538+698, 20+538+699, 20+538+711, 20+538+754, 20+538+760, 20+538+781, 20+538+786, 20+538+797, 20+538+834, 20+538+835, 20+598+602, 20+598+605, 20+598+609, 20+598+676, 20+598+694, 20+598+698, 20+598+699, 20+598+711, 20+598+754, 20+598+760, 20+598+781, 20+598+786, 20+598+797, 20+598+834, 20+598+835, 20+602+605, 20+602+609, 20+602+676, 20+602+694, 20+602+698, 20+602+699, 20+602+711, 20+602+754, 20+602+760, 20+602+781, 20+602+786, 20+602+797, 20+602+834, 20+602+835, 20+605+609, 20+605+676, 20+605+694, 20+605+698, 20+605+699, 20+605+711, 20+605+754, 20+605+760, 20+605+781, 20+605+786, 20+605+797, 20+605+834, 20+605+835, 20+609+676, 20+609+694, 20+609+698, 20+609+699, 20+609+711, 20+609+754, 20+609+760, 20+609+781, 20+609+786, 20+609+797, 20+609+834, 20+609+835, 20+676+694, 20+676+698, 20+676+699, 20+676+711, 20+676+754, 20+676+760, 20+676+781, 20+676+786, 20+676+797, 20+676+834, 20+676+835, 20+694+698, 20+694+699, 20+694+711, 20+694+754, 20+694+760, 20+694+781, 20+694+786, 20+694+797, 20+694+834, 20+694+835, 20+698+699, 20+698+711, 20+698+754, 20+698+760, 20+698+781, 20+698+786, 20+698+797, 20+698+834, 20+698+835, 20+699+711, 20+699+754, 20+699+760, 20+699+781, 20+699+786, 20+699+797, 20+699+834, 20+699+835, 20+711+754, 20+711+760, 20+711+781, 20+711+786, 20+711+797, 20+711+834, 20+711+835, 20+754+760, 20+754+781, 20+754+786, 20+754+797, 20+754+834, 20+754+835, 20+760+781, 20+760+786, 20+760+797, 20+760+834, 20+760+835, 20+781+786, 20+781+797, 20+781+834, 20+781+835, 20+786+797, 20+786+834, 20+786+835, 20+797+834, 20+797+835, 20+834+835, 51+53+55, 51+53+56, 51+53+60, 51+53+63, 51+53+79 51+53+87, 51+53+192, 51+53+302, 51+53+387, 51+53+388, 51+53+390, 51+53+403, 51+53+408, 51+53+410, 51+53+416, 51+53+448, 51+53+451, 51+53+471, 51+53+472, 51+53+507, 51+53+512, 51+53+515, 51+53+538, 51+53+598, 51+53+602, 51+53+605, 51+53+609, 51+53+676, 51+53+694, 51+53+698, 51+53+699, 51+53+711, 51+53+754, 51+53+760, 51+53+781, 51+53+786, 51+53+797, 51+53+834, 51+53+835, 51+55+56, 51+55+60, 51+55+63, 51+55+79, 51+55+87, 51+55+192, 51+55+302, 51+55+387, 51+55+388, 51+55+390, 51+55+403, 51+55+408, 51+55+410, 51+55+416, 51+55+448, 51+55+451, 51+55+471, 51+55+472, 51+55+507, 51+55+512, 51+55+515, 51+55+538, 51+55+598, 51+55+602, 51+55+605, 51+55+609, 51+55+676, 51+55+694, 51+55+698, 51+55+699, 51+55+711, 51+55+754, 51+55+760, 51+55+781, 51+55+786, 51+55+797, 51+55+834, 51+55+835, 51+56+60, 51+56+63, 51+56+79, 51+56+87, 51+56+192, 51+56+302, 51+56+387, 51+56+388, 51+56+390, 51+56+403, 51+56+408, 51+56+410, 51+56+416, 51+56+448, 51+56+451, 51+56+471, 51+56+472, 51+56+507, 51+56+512, 51+56+515, 51+56+538, 51+56+598, 51+56+602, 51+56+605, 51+56+609, 51+56+676, 51+56+694, 51+56+698, 51+56+699, 51+56+711, 51+56+754, 51+56+760, 51+56+781, 51+56+786, 51+56+797, 51+56+834, 51+56+835, 51+60+63, 51+60+79, 51+60+87, 51+60+192, 51+60+302, 51+60+387, 51+60+388, 51+60+390, 51+60+403, 51+60+408, 51+60+410, 51+60+416, 51+60+448, 51+60+451, 51+60+471, 51+60+472, 51+60+507, 51+60+512, 51+60+515, 51+60+538, 51+60+598, 51+60+602, 51+60+605, 51+60+609, 51+60+676, 51+60+694, 51+60+698, 51+60+699, 51+60+711, 51+60+754, 51+60+760, 51+60+781, 51+60+786, 51+60+797, 51+60+834, 51+60+835, 51+63+79, 51+63+87, 51+63+192, 51+63+302, 51+63+387, 51+63+388, 51+63+390, 51+63+403, 51+63+408, 51+63+410, 51+63+416, 51+63+448, 51+63+451, 51+63+471, 51+63+472, 51+63+507, 51+63+512, 51+63+515, 51+63+538, 51+63+598, 51+63+602, 51+63+605, 51+63+609, 51+63+676, 51+63+694, 51+63+698, 51+63+699, 51+63+711, 51+63+754, 51+63+760, 51+63+781, 51+63+786, 51+63+797, 51+63+834, 51+63+835, 51+79+87, 51+79+192, 51+79+302, 51+79+387, 51+79+388, 51+79+390, 51+79+403, 51+79+408, 51+79+410, 51+79+416, 51+79+448, 51+79+451, 51+79+471, 51+79+472, 51+79+507, 51+79+512, 51+79+515, 51+79+538, 51+79+598, 51+79+602, 51+79+605, 51+79+609, 51+79+676, 51+79+694, 51+79+698, 51+79+699, 51+79+711, 51+79+754, 51+79+760, 51+79+781, 51+79+786, 51+79+797, 51+79+834, 51+79+835, 51+87+192, 51+87+302, 51+87+387, 51+87+388, 51+87+390, 51+87+403, 51+87+408, 51+87+410, 51+87+416, 51+87+448, 51+87+451, 51+87+471, 51+87+472, 51+87+507, 51+87+512, 51+87+515, 51+87+538, 51+87+598, 51+87+602, 51+87+605, 51+87+609, 51+87+676, 51+87+694, 51+87+698, 51+87+699, 51+87+711, 51+87+754, 51+87+760, 51+87+781, 51+87+786, 51+87+797, 51+87+834, 51+87+835, 51+192+302, 51+192+387, 51+192+388, 51+192+390, 51+192+403, 51+192+408, 51+192+410, 51+192+416, 51+192+448, 51+192+451, 51+192+471, 51+192+472, 51+192+507, 51+192+512, 51+192+515, 51+192+538, 51+192+598, 51+192+602, 51+192+605, 51+192+609, 51+192+676, 51+192+694, 51+192+698, 51+192+699, 51+192+711, 51+192+754, 51+192+760, 51+192+781, 51+192+786, 51+192+797, 51+192+834, 51+192+835, 51+302+387, 51+302+388, 51+302+390, 51+302+403, 51+302+408, 51+302+410, 51+302+416, 51+302+448, 51+302+451, 51+302+471, 51+302+472, 51+302+507, 51+302+512, 51+302+515, 51+302+538, 51+302+598, 51+302+602, 51+302+605, 51+302+609, 51+302+676, 51+302+694, 51+302+698, 51+302+699, 51+302+711, 51+302+754, 51+302+760, 51+302+781, 51+302+786, 51+302+797, 51+302+834, 51+302+835, 51+387+388, 51+387+390, 51+387+403, 51+387+408, 51+387+410, 51+387+416, 51+387+448, 51+387+451, 51+387+471, 51+387+472, 51+387+507, 51+387+512, 51+387+515, 51+387+538, 51+387+598, 51+387+602, 51+387+605, 51+387+609, 51+387+676, 51+387+694, 51+387+698, 51+387+699, 51+387+711, 51+387+754, 51+387+760, 51+387+781, 51+387+786, 51+387+797, 51+387+834, 51+387+835, 51+388+390, 51+388+403, 51+388+408, 51+388+410, 51+388+416, 51+388+448, 51+388+451, 51+388+471, 51+388+472, 51+388+507, 51+388+512, 51+388+515, 51+388+538, 51+388+598, 51+388+602, 51+388+605, 51+388+609, 51+388+676, 51+388+694, 51+388+698, 51+388+699, 51+388+711, 51+388+754, 51+388+760, 51+388+781, 51+388+786, 51+388+797, 51+388+834, 51+388+835, 51+390+403, 51+390+408, 51+390+410, 51+390+416, 51+390+448, 51+390+451, 51+390+471, 51+390+472, 51+390+507, 51+390+512, 51+390+515, 51+390+538, 51+390+598, 51+390+602, 51+390+605, 51+390+609, 51+390+676, 51+390+694, 51+390+698, 51+390+699, 51+390+711, 51+390+754, 51+390+760, 51+390+781, 51+390+786, 51+390+797, 51+390+834, 51+390+835, 51+403+408, 51+403+410, 51+403+416, 51+403+448, 51+403+451, 51+403+471, 51+403+472, 51+403+507, 51+403+512, 51+403+515, 51+403+538, 51+403+598, 51+403+602, 51+403+605, 51+403+609, 51+403+676, 51+403+694, 51+403+698, 51+403+699, 51+403+711, 51+403+754, 51+403+760, 51+403+781, 51+403+786, 51+403+797, 51+403+834, 51+403+835, 51+408+410, 51+408+416, 51+408+448, 51+408+451, 51+408+471, 51+408+472, 51+408+507, 51+408+512, 51+408+515, 51+408+538, 51+408+598, 51+408+602, 51+408+605, 51+408+609, 51+408+676, 51+408+694, 51+408+698, 51+408+699, 51+408+711, 51+408+754, 51+408+760, 51+408+781, 51+408+786, 51+408+797, 51+408+834, 51+408+835, 51+410+416, 51+410+448, 51+410+451, 51+410+471, 51+410+472, 51+410+507, 51+410+512, 51+410+515, 51+410+538, 51+410+598, 51+410+602, 51+410+605, 51+410+609, 51+410+676, 51+410+694, 51+410+698, 51+410+699, 51+410+711, 51+410+754, 51+410+760, 51+410+781, 51+410+786, 51+410+797, 51+410+834, 51+410+835, 51+416+448, 51+416+451, 51+416+471, 51+416+472, 51+416+507, 51+416+512, 51+416+515, 51+416+538, 51+416+598, 51+416+602, 51+416+605, 51+416+609, 51+416+676, 51+416+694, 51+416+698, 51+416+699, 51+416+711, 51+416+754, 51+416+760, 51+416+781, 51+416+786, 51+416+797, 51+416+834, 51+416+835, 51+448+451, 51+448+471, 51+448+472, 51+448+507, 51+448+512, 51+448+515, 51+448+538, 51+448+598, 51+448+602, 51+448+605, 51+448+609, 51+448+676, 51+448+694, 51+448+698, 51+448+699, 51+448+711, 51+448+754, 51+448+760, 51+448+781, 51+448+786, 51+448+797, 51+448+834, 51+448+835, 51+451+471, 51+451+472, 51+451+507, 51+451+512, 51+451+515, 51+451+538, 51+451+598, 51+451+602, 51+451+605, 51+451+609, 51+451+676, 51+451+694, 51+451+698, 51+451+699, 51+451+711, 51+451+754, 51+451+760, 51+451+781, 51+451+786, 51+451+797, 51+451+834, 51+451+835, 51+471+472, 51+471+507, 51+471+512, 51+471+515, 51+471+538, 51+471+598, 51+471+602, 51+471+605, 51+471+609, 51+471+676, 51+471+694, 51+471+698, 51+471+699, 51+471+711, 51+471+754, 51+471+760, 51+471+781, 51+471+786, 51+471+797, 51+471+834, 51+471+835, 51+472+507, 51+472+512, 51+472+515, 51+472+538, 51+472+598, 51+472+602, 51+472+605, 51+472+609, 51+472+676, 51+472+694, 51+472+698, 51+472+699, 51+472+711, 51+472+754, 51+472+760, 51+472+781, 51+472+786, 51+472+797, 51+472+834, 51+472+835, 51+507+512, 51+507+515, 51+507+538, 51+507+598, 51+507+602, 51+507+605, 51+507+609, 51+507+676, 51+507+694, 51+507+698, 51+507+699, 51+507+711, 51+507+754, 51+507+760, 51+507+781, 51+507+786, 51+507+797, 51+507+834, 51+507+835, 51+512+515, 51+512+538, 51+512+598, 51+512+602, 51+512+605, 51+512+609, 51+512+676, 51+512+694, 51+512+698, 51+512+699, 51+512+711, 51+512+754, 51+512+760, 51+512+781, 51+512+786, 51+512+797, 51+512+834, 51+512+835, 51+515+538, 51+515+598, 51+515+602, 51+515+605, 51+515+609, 51+515+676, 51+515+694, 51+515+698, 51+515+699, 51+515+711, 51+515+754, 51+515+760, 51+515+781, 51+515+786, 51+515+797, 51+515+834, 51+515+835, 51+538+598, 51+538+602, 51+538+605, 51+538+609, 51+538+676, 51+538+694, 51+538+698, 51+538+699, 51+538+711, 51+538+754, 51+538+760, 51+538+781, 51+538+786, 51+538+797, 51+538+834, 51+538+835, 51+598+602, 51+598+60551+598+609, 51+598+676, 51+598+694, 51+598+698, 51+598+699, 51+598+711, 51+598+754, 51+598+760, 51+598+781 51+598+786, 51+598+797, 51+598+834, 51+598+835, 51+602+605, 51+602+609, 51+602+676, 51+602+694, 51+602+698, 51+602+699, 51+602+711, 51+602+754, 51+602+760, 51+602+781, 51+602+786, 51+602+797, 51+602+834, 51+602+835, 51+605+609, 51+605+676, 51+605+694, 51+605+698, 51+605+699, 51+605+711, 51+605+754, 51+605+760, 51+605+781, 51+605+786, 51+605+797, 51+605+834, 51+605+835, 51+609+676, 51+609+694, 51+609+698, 51+609+699, 51+609+711, 51+609+754, 51+609+760, 51+609+781, 51+609+786, 51+609+797, 51+609+834, 51+609+835, 51+676+694, 51+676+698, 51+676+699, 51+676+711, 51+676+754, 51+676+760, 51+676+781, 51+676+786, 51+676+797, 51+676+834, 51+676+835, 51+694+698, 51+694+699, 51+694+711, 51+694+754, 51+694+760, 51+694+781, 51+694+786, 51+694+797, 51+694+834, 51+694+835, 51+698+699, 51+698+711, 51+698+754, 51+698+760, 51+698+781, 51+698+786, 51+698+797, 51+698+834, 51+698+835, 51+699+711, 51+699+754, 51+699+760, 51+699+781, 51+699+786, 51+699+797, 51+699+834, 51+699+835, 51+711+754, 51+711+760, 51+711+781, 51+711+786, 51+711+797, 51+711+834, 51+711+835, 51+754+760, 51+754+781, 51+754+786, 51+754+797, 51+754+834, 51+754+835, 51+760+781, 51+760+786, 51+760+797, 51+760+834, 51+760+835, 51+781+786, 51+781+797, 51+781+834, 51+781+835, 51+786+797, 51+786+834, 51+786+835, 51+797+834, 51+797+835, 51+834+835, 53+55+56, 53+55+60, 53+55+63, 53+55+79, 53+55+87, 53+55+192, 53+55+302, 53+55+387, 53+55+388, 53+55+390, 53+55+403, 53+55+408, 53+55+410, 53+55+416, 53+55+448, 53+55+451, 53+55+471, 53+55+472, 53+55+507, 53+55+512, 53+55+515, 53+55+538, 53+55+598, 53+55+602, 53+55+605, 53+55+609, 53+55+676, 53+55+694, 53+55+698, 53+55+699, 53+55+711, 53+55+754, 53+55+760, 53+55+781, 53+55+786, 53+55+797, 53+55+834, 53+55+835, 53+56+60, 53+56+63, 53+56+79, 53+56+87, 53+56+192, 53+56+302, 53+56+387, 53+56+388, 53+56+390, 53+56+403, 53+56+408, 53+56+410, 53+56+416, 53+56+448, 53+56+451, 53+56+471, 53+56+472, 53+56+507, 53+56+512, 53+56+515, 53+56+538, 53+56+598, 53+56+602, 53+56+605, 53+56+609, 53+56+676, 53+56+694, 53+56+698, 53+56+699, 53+56+711, 53+56+754, 53+56+760, 53+56+781, 53+56+786, 53+56+797, 53+56+834, 53+56+835, 53+60+63, 53+60+79, 53+60+87, 53+60+192, 53+60+302, 53+60+387, 53+60+388, 53+60+390, 53+60+403, 53+60+408, 53+60+410, 53+60+416, 53+60+448, 53+60+451, 53+60+471, 53+60+472, 53+60+507, 53+60+512, 53+60+515, 53+60+538, 53+60+598, 53+60+602, 53+60+605, 53+60+609, 53+60+676, 53+60+694, 53+60+698, 53+60+699, 53+60+711, 53+60+754, 53+60+760, 53+60+781, 53+60+786, 53+60+797, 53+60+834, 53+60+835, 53+63+79, 53+63+87, 53+63+192, 53+63+302, 53+63+387, 53+63+388, 53+63+390, 53+63+403, 53+63+408, 53+63+410, 53+63+416, 53+63+448, 53+63+451, 53+63+471, 53+63+472, 53+63+507, 53+63+512, 53+63+515, 53+63+538, 53+63+598, 53+63+602, 53+63+605, 53+63+609, 53+63+676, 53+63+694, 53+63+698, 53+63+699, 53+63+711, 53+63+754, 53+63+760, 53+63+781, 53+63+786, 53+63+797, 53+63+834, 53+63+835, 53+79+87, 53+79+192, 53+79+302, 53+79+387, 53+79+388, 53+79+390, 53+79+403, 53+79+408, 53+79+410, 53+79+416, 53+79+448, 53+79+451, 53+79+471, 53+79+472, 53+79+507, 53+79+512, 53+79+515, 53+79+538, 53+79+598, 53+79+602, 53+79+605, 53+79+609, 53+79+676, 53+79+694, 53+79+698, 53+79+699, 53+79+711, 53+79+754, 53+79+760, 53+79+781, 53+79+786, 53+79+797, 53+79+834, 53+79+835, 53+87+192, 53+87+302, 53+87+387, 53+87+388, 53+87+390, 53+87+403, 53+87+408, 53+87+410, 53+87+416, 53+87+448, 53+87+451, 53+87+471, 53+87+472, 53+87+507, 53+87+512, 53+87+515, 53+87+538, 53+87+598, 53+87+602, 53+87+605, 53+87+609, 53+87+676, 53+87+694, 53+87+698, 53+87+699, 53+87+711, 53+87+754, 53+87+760, 53+87+781, 53+87+786, 53+87+797, 53+87+834, 53+87+835, 53+192+302, 53+192+387, 53+192+388, 53+192+390, 53+192+403, 53+192+408, 53+192+410, 53+192+416, 53+192+448, 53+192+451, 53+192+471, 53+192+472, 53+192+507, 53+192+512, 53+192+515, 53+192+538, 53+192+598, 53+192+602, 53+192+605, 53+192+609, 53+192+676, 53+192+694, 53+192+698, 53+192+699, 53+192+711, 53+192+754, 53+192+760, 53+192+781, 53+192+786, 53+192+797, 53+192+834, 53+192+835, 53+302+387, 53+302+388, 53+302+390, 53+302+403, 53+302+408, 53+302+410, 53+302+416, 53+302+448, 53+302+451, 53+302+471, 53+302+472, 53+302+507, 53+302+512, 53+302+515, 53+302+538, 53+302+598, 53+302+602, 53+302+605, 53+302+609, 53+302+676, 53+302+694, 53+302+698, 53+302+699, 53+302+711, 53+302+754, 53+302+760, 53+302+781, 53+302+786, 53+302+797, 53+302+834, 53+302+835, 53+387+388, 53+387+390, 53+387+403, 53+387+408, 53+387+410, 53+387+416, 53+387+448, 53+387+451, 53+387+471, 53+387+472, 53+387+507, 53+387+512, 53+387+515, 53+387+538, 53+387+598, 53+387+602, 53+387+605, 53+387+609, 53+387+676, 53+387+694, 53+387+698, 53+387+699, 53+387+711, 53+387+754, 53+387+760, 53+387+781, 53+387+786, 53+387+797, 53+387+834, 53+387+835, 53+388+390, 53+388+403, 53+388+408, 53+388+410, 53+388+416, 53+388+448, 53+388+451, 53+388+471, 53+388+472, 53+388+507, 53+388+512, 53+388+515, 53+388+538, 53+388+598, 53+388+602, 53+388+605, 53+388+609, 53+388+676, 53+388+694, 53+388+698, 53+388+699, 53+388+711, 53+388+754, 53+388+760, 53+388+781, 53+388+786, 53+388+797, 53+388+834, 53+388+835, 53+390+403, 53+390+408, 53+390+410, 53+390+416, 53+390+448, 53+390+451, 53+390+471, 53+390+472, 53+390+507, 53+390+512, 53+390+515, 53+390+538, 53+390+598, 53+390+602, 53+390+605, 53+390+609, 53+390+676, 53+390+694, 53+390+698, 53+390+699, 53+390+711, 53+390+754, 53+390+760, 53+390+781, 53+390+786, 53+390+797, 53+390+834, 53+390+835, 53+403+408, 53+403+410, 53+403+416, 53+403+448, 53+403+451, 53+403+471, 53+403+472, 53+403+507, 53+403+512, 53+403+515, 53+403+538, 53+403+598, 53+403+602, 53+403+605, 53+403+609, 53+403+676, 53+403+694, 53+403+698, 53+403+699, 53+403+711, 53+403+754, 53+403+760, 53+403+781, 53+403+786, 53+403+797, 53+403+834, 53+403+835, 53+408+410, 53+408+416, 53+408+448, 53+408+451, 53+408+471, 53+408+472, 53+408+507, 53+408+512, 53+408+515, 53+408+538, 53+408+598, 53+408+602, 53+408+605, 53+408+609, 53+408+676, 53+408+694, 53+408+69853+408+699, 53+408+711, 53+408+754, 53+408+760, 53+408+781, 53+408+786, 53+408+797, 53+408+834, 53+408+835, 53+410+416, 53+410+448, 53+410+451, 53+410+471, 53+410+472, 53+410+507, 53+410+512, 53+410+515, 53+410+538, 53+410+598, 53+410+602, 53+410+605, 53+410+609, 53+410+676, 53+410+694, 53+410+698, 53+410+699, 53+410+711, 53+410+754, 53+410+760, 53+410+781, 53+410+786, 53+410+797, 53+410+834, 53+410+835, 53+416+448, 53+416+451, 53+416+471, 53+416+472, 53+416+507, 53+416+512, 53+416+515, 53+416+538, 53+416+598, 53+416+602, 53+416+605, 53+416+609, 53+416+676, 53+416+694, 53+416+698, 53+416+699, 53+416+711, 53+416+754, 53+416+760, 53+416+781, 53+416+786, 53+416+797, 53+416+834, 53+416+835, 53+448+451, 53+448+471, 53+448+472, 53+448+507, 53+448+512, 53+448+515, 53+448+538, 53+448+598, 53+448+602, 53+448+605, 53+448+609, 53+448+676, 53+448+694, 53+448+698, 53+448+699, 53+448+711, 53+448+754, 53+448+760, 53+448+781, 53+448+786, 53+448+797, 53+448+834, 53+448+835, 53+451+471, 53+451+472, 53+451+507, 53+451+512, 53+451+515, 53+451+538, 53+451+598, 53+451+602, 53+451+605, 53+451+609, 53+451+676, 53+451+694, 53+451+698, 53+451+699, 53+451+711, 53+451+754, 53+451+760, 53+451+781, 53+451+786, 53+451+797, 53+451+834, 53+451+835, 53+471+472, 53+471+507, 53+471+512, 53+471+515, 53+471+538, 53+471+598, 53+471+602, 53+471+605, 53+471+609, 53+471+676, 53+471+694, 53+471+698, 53+471+699, 53+471+711, 53+471+754, 53+471+760, 53+471+781, 53+471+786, 53+471+797, 53+471+834, 53+471+835, 53+472+507, 53+472+512, 53+472+515, 53+472+538, 53+472+598, 53+472+602, 53+472+605, 53+472+609, 53+472+676, 53+472+694, 53+472+698, 53+472+699, 53+472+711, 53+472+754, 53+472+760, 53+472+781, 53+472+786, 53+472+797, 53+472+834, 53+472+835, 53+507+512, 53+507+515, 53+507+538, 53+507+598, 53+507+602, 53+507+605, 53+507+609, 53+507+676, 53+507+694, 53+507+698, 53+507+699, 53+507+711, 53+507+754, 53+507+760, 53+507+781, 53+507+786, 53+507+797, 53+507+834, 53+507+835, 53+512+515, 53+512+538, 53+512+598, 53+512+602, 53+512+605, 53+512+609, 53+512+676, 53+512+694, 53+512+698, 53+512+699, 53+512+711, 53+512+754, 53+512+760, 53+512+781, 53+512+786, 53+512+797, 53+512+834, 53+512+835, 53+515+538, 53+515+598, 53+515+602, 53+515+605, 53+515+609, 53+515+676, 53+515+694, 53+515+698, 53+515+699, 53+515+711, 53+515+754, 53+515+760, 53+515+781, 53+515+786, 53+515+797, 53+515+834, 53+515+835, 53+538+598, 53+538+602, 53+538+605, 53+538+609, 53+538+676, 53+538+694, 53+538+698, 53+538+699, 53+538+711, 53+538+754, 53+538+760, 53+538+781, 53+538+786, 53+538+797, 53+538+834, 53+538+835, 53+598+602, 53+598+605, 53+598+609, 53+598+676, 53+598+694, 53+598+698, 53+598+699, 53+598+711, 53+598+754, 53+598+760, 53+598+781, 53+598+786, 53+598+797, 53+598+834, 53+598+835, 53+602+605, 53+602+609, 53+602+676, 53+602+694, 53+602+698, 53+602+699, 53+602+711, 53+602+754, 53+602+760, 53+602+781, 53+602+786, 53+602+797, 53+602+834, 53+602+835, 53+605+609, 53+605+676, 53+605+694, 53+605+698, 53+605+699, 53+605+711, 53+605+754, 53+605+760, 53+605+781, 53+605+786, 53+605+797, 53+605+834, 53+605+835, 53+609+676, 53+609+694, 53+609+698, 53+609+699, 53+609+711, 53+609+754, 53+609+760, 53+609+781, 53+609+786, 53+609+797, 53+609+834, 53+609+835, 53+676+694, 53+676+698, 53+676+699, 53+676+711, 53+676+754, 53+676+760, 53+676+781, 53+676+786, 53+676+797, 53+676+834, 53+676+835, 53+694+698, 53+694+699, 53+694+711, 53+694+754, 53+694+760, 53+694+781, 53+694+786, 53+694+797, 53+694+834, 53+694+835, 53+698+699, 53+698+711, 53+698+754, 53+698+760, 53+698+781, 53+698+786, 53+698+797, 53+698+834, 53+698+835, 53+699+711, 53+699+754, 53+699+760, 53+699+781, 53+699+786, 53+699+797, 53+699+834, 53+699+835, 53+711+754, 53+711+760, 53+711+781, 53+711+786, 53+711+797, 53+711+834, 53+711+835, 53+754+760, 53+754+781, 53+754+786, 53+754+797, 53+754+834, 53+754+835, 53+760+781, 53+760+786, 53+760+797, 53+760+834, 53+760+835, 53+781+786, 53+781+797, 53+781+834, 53+781+835, 53+786+797, 53+786+834, 53+786+835, 53+797+834, 53+797+835, 53+834+835, 55+56+60, 55+56+63, 55+56+79, 55+56+87, 55+56+192, 55+56+302, 55+56+387, 55+56+388, 55+56+390, 55+56+403, 55+56+408, 55+56+410, 55+56+416, 55+56+448, 55+56+451, 55+56+471, 55+56+472, 55+56+507, 55+56+512, 55+56+515, 55+56+538, 55+56+598, 55+56+602, 55+56+605, 55+56+609, 55+56+676, 55+56+694, 55+56+698, 55+56+699, 55+56+711, 55+56+754, 55+56+760, 55+56+781, 55+56+786, 55+56+797, 55+56+834, 55+56+835, 55+60+63, 55+60+79, 55+60+87, 55+60+192, 55+60+302, 55+60+387, 55+60+388, 55+60+390, 55+60+403, 55+60+408, 55+60+410, 55+60+416, 55+60+448, 55+60+451, 55+60+471, 55+60+472, 55+60+507, 55+60+512, 55+60+515, 55+60+538, 55+60+598, 55+60+602, 55+60+605, 55+60+609,5+60+676, 55+60+694, 55+60+698, 55+60+699, 55+60+711, 55+60+754, 55+60+760, 55+60+781, 55+60+786, 55+60+797, 55+60+834, 55+60+835, 55+63+79, 55+63+87, 55+63+192, 55+63+302, 55+63+387, 55+63+388, 55+63+390, 55+63+403, 55+63+408, 55+63+410, 55+63+416, 55+63+448, 55+63+451, 55+63+471, 55+63+472, 55+63+507, 55+63+512, 55+63+515, 55+63+538, 55+63+598, 55+63+602, 55+63+605, 55+63+609, 55+63+676, 55+63+694, 55+63+698, 55+63+699, 55+63+711, 55+63+754, 55+63+760, 55+63+781, 55+63+786, 55+63+797, 55+63+834, 55+63+835, 55+79+87, 55+79+192, 55+79+302, 55+79+387, 55+79+388, 55+79+390, 55+79+403, 55+79+408, 55+79+410, 55+79+416, 55+79+448, 55+79+451, 55+79+471, 55+79+472, 55+79+507, 55+79+512, 55+79+515, 55+79+538, 55+79+598, 55+79+602, 55+79+605, 55+79+609, 55+79+676, 55+79+694, 55+79+698, 55+79+699, 55+79+711, 55+79+754, 55+79+760, 55+79+781, 55+79+786, 55+79+797, 55+79+834, 55+79+835, 55+87+192, 55+87+302, 55+87+387, 55+87+388, 55+87+390, 55+87+403, 55+87+408, 55+87+410, 55+87+416, 55+87+448, 55+87+451, 55+87+471, 55+87+472, 55+87+507, 55+87+512, 55+87+515, 55+87+538, 55+87+598, 55+87+602, 55+87+605, 55+87+609, 55+87+676, 55+87+694, 55+87+698, 55+87+699, 55+87+711, 55+87+754, 55+87+760, 55+87+781, 55+87+786, 55+87+797, 55+87+834, 55+87+835, 55+192+302, 55+192+387, 55+192+388, 55+192+390, 55+192+403, 55+192+408, 55+192+410, 55+192+416, 55+192+448, 55+192+451, 55+192+471, 55+192+472, 55+192+507, 55+192+512, 55+192+515, 55+192+538, 55+192+598, 55+192+602, 55+192+605, 55+192+609, 55+192+676, 55+192+694, 55+192+698, 55+192+699, 55+192+711, 55+192+754, 55+192+760, 55+192+781, 55+192+786, 55+192+797, 55+192+834, 55+192+835, 55+302+387, 55+302+388, 55+302+390, 55+302+403, 55+302+408, 55+302+410, 55+302+416, 55+302+448, 55+302+451, 55+302+471, 55+302+472, 55+302+507, 55+302+512, 55+302+515, 55+302+538, 55+302+598, 55+302+602, 55+302+605, 55+302+609, 55+302+676, 55+302+694, 55+302+698, 55+302+699, 55+302+711, 55+302+754, 55+302+760, 55+302+781, 55+302+786, 55+302+797, 55+302+834, 55+302+835, 55+387+388, 55+387+390, 55+387+403, 55+387+408, 55+387+410, 55+387+416, 55+387+448, 55+387+451, 55+387+471, 55+387+472, 55+387+507, 55+387+512, 55+387+515, 55+387+538, 55+387+598, 55+387+602, 55+387+605, 55+387+609, 55+387+676, 55+387+694, 55+387+698, 55+387+699, 55+387+711, 55+387+754, 55+387+760, 55+387+781, 55+387+786, 55+387+797, 55+387+834, 55+387+835, 55+388+390, 55+388+403, 55+388+408, 55+388+410, 55+388+416, 55+388+448, 55+388+451, 55+388+471, 55+388+472, 55+388+507, 55+388+512, 55+388+515, 55+388+538, 55+388+598, 55+388+602, 55+388+605, 55+388+609, 55+388+676, 55+388+694, 55+388+698, 55+388+699, 55+388+711, 55+388+754, 55+388+760, 55+388+781, 55+388+786, 55+388+797, 55+388+834, 55+388+835, 55+390+403, 55+390+408, 55+390+410, 55+390+416, 55+390+448, 55+390+451, 55+390+471, 55+390+472, 55+390+507, 55+390+512, 55+390+515, 55+390+538, 55+390+598, 55+390+602, 55+390+605, 55+390+609, 55+390+676, 55+390+694, 55+390+698, 55+390+699, 55+390+711, 55+390+754, 55+390+760, 55+390+781, 55+390+786, 55+390+797, 55+390+834, 55+390+835, 55+403+408, 55+403+410, 55+403+416, 55+403+448, 55+403+451, 55+403+471, 55+403+472, 55+403+507, 55+403+512, 55+403+515, 55+403+538, 55+403+598, 55+403+602, 55+403+605, 55+403+609, 55+403+676, 55+403+694, 55+403+698, 55+403+699, 55+403+711, 55+403+754, 55+403+760, 55+403+781, 55+403+786, 55+403+797, 55+403+834, 55+403+835, 55+408+410, 55+408+416, 55+408+448, 55+408+451, 55+408+471, 55+408+472, 55+408+507, 55+408+512, 55+408+515, 55+408+538, 55+408+598, 55+408+602, 55+408+605, 55+408+609, 55+408+676, 55+408+694, 55+408+698, 55+408+699, 55+408+711, 55+408+754, 55+408+760, 55+408+781, 55+408+786, 55+408+797, 55+408+834, 55+408+835, 55+410+416, 55+410+448, 55+410+451, 55+410+471, 55+410+472, 55+410+507, 55+410+512, 55+410+515, 55+410+538, 55+410+598, 55+410+602, 55+410+605, 55+410+609, 55+410+676, 55+410+694, 55+410+698, 55+410+699, 55+410+711, 55+410+754, 55+410+760, 55+410+781, 55+410+786, 55+410+797, 55+410+834, 55+410+835, 55+416+448, 55+416+451, 55+416+471, 55+416+472, 55+416+507, 55+416+512, 55+416+515, 55+416+538, 55+416+598, 55+416+602, 55+416+605, 55+416+609, 55+416+676, 55+416+694, 55+416+698, 55+416+699, 55+416+711, 55+416+754, 55+416+760, 55+416+781, 55+416+786, 55+416+797, 55+416+834, 55+416+835, 55+448+451, 55+448+471, 55+448+472, 55+448+507, 55+448+512, 55+448+515, 55+448+538, 55+448+598, 55+448+602, 55+448+605, 55+448+609, 55+448+676, 55+448+694, 55+448+698, 55+448+699, 55+448+711, 55+448+754, 55+448+760, 55+448+781, 55+448+786, 55+448+797, 55+448+834, 55+448+835, 55+451+471, 55+451+472, 55+451+507, 55+451+512, 55+451+515, 55+451+538, 55+451+598, 55+451+602, 55+451+605, 55+451+609, 55+451+676, 55+451+694, 55+451+698, 55+451+699, 55+451+711, 55+451+754, 55+451+760, 55+451+781, 55+451+786, 55+451+797, 55+451+834, 55+451+835, 55+471+472, 55+471+507, 55+471+512, 55+471+515, 55+471+538, 55+471+598, 55+471+602, 55+471+605, 55+471+609, 55+471+676, 55+471+694, 55+471+698, 55+471+699, 55+471+711, 55+471+754, 55+471+760, 55+471+781, 55+471+786, 55+471+797, 55+471+834, 55+471+835, 55+472+507, 55+472+512, 55+472+515, 55+472+538, 55+472+598, 55+472+602, 55+472+605, 55+472+609, 55+472+676, 55+472+694, 55+472+698, 55+472+699, 55+472+711, 55+472+754, 55+472+760, 55+472+781, 55+472+786, 55+472+797, 55+472+834, 55+472+835, 55+507+512, 55+507+515, 55+507+538, 55+507+598, 55+507+602, 55+507+605, 55+507+609, 55+507+676, 55+507+694, 55+507+698, 55+507+699, 55+507+711, 55+507+754, 55+507+760, 55+507+781, 55+507+786, 55+507+797, 55+507+834, 55+507+835, 55+512+515, 55+512+538, 55+512+598, 55+512+602, 55+512+605, 55+512+609, 55+512+676, 55+512+694, 55+512+698, 55+512+699, 55+512+711, 55+512+754, 55+512+760, 55+512+781, 55+512+786, 55+512+797, 55+512+834, 55+512+835, 55+515+538, 55+515+598, 55+515+602, 55+515+605, 55+515+609, 55+515+676, 55+515+694, 55+515+698, 55+515+699, 55+515+711, 55+515+754, 55+515+760, 55+515+781, 55+515+786, 55+515+797, 55+515+834, 55+515+835, 55+538+598, 55+538+602, 55+538+605, 55+538+609, 55+538+676, 55+538+694, 55+538+698, 55+538+699, 55+538+711, 55+538+754, 55+538+760, 55+538+781, 55+538+786, 55+538+797, 55+538+834, 55+538+835, 55+598+602, 55+598+605, 55+598+609, 55+598+676, 55+598+694, 55+598+698, 55+598+699, 55+598+711, 55+598+754, 55+598+760, 55+598+781, 55+598+786, 55+598+797, 55+598+834, 55+598+835, 55+602+605, 55+602+609, 55+602+676, 55+602+694, 55+602+698, 55+602+699, 55+602+711, 55+602+754, 55+602+760, 55+602+781, 55+602+786, 55+602+797, 55+602+834, 55+602+835, 55+605+609, 55+605+676, 55+605+694, 55+605+698, 55+605+699, 55+605+711, 55+605+754, 55+605+760, 55+605+781, 55+605+786, 55+605+797, 55+605+834, 55+605+835, 55+609+676, 55+609+694, 55+609+698, 55+609+699, 55+609+711, 55+609+754, 55+609+760, 55+609+781, 55+609+786, 55+609+797, 55+609+834, 55+609+835, 55+676+694, 55+676+698, 55+676+699, 55+676+711, 55+676+754, 55+676+760, 55+676+781, 55+676+786, 55+676+797, 55+676+834, 55+676+835, 55+694+698, 55+694+699, 55+694+711, 55+694+754, 55+694+760, 55+694+781, 55+694+786, 55+694+797, 55+694+834, 55+694+835, 55+698+699, 55+698+711, 55+698+754, 55+698+760, 55+698+781, 55+698+786, 55+698+797, 55+698+834, 55+698+835, 55+699+711, 55+699+754, 55+699+760, 55+699+781, 55+699+786, 55+699+797, 55+699+834, 55+699+835, 55+711+754, 55+711+760, 55+711+781, 55+711+786, 55+711+797, 55+711+834, 55+711+835, 55+754+760, 55+754+781, 55+754+786, 55+754+797, 55+754+834, 55+754+835, 55+760+781, 55+760+786, 55+760+797, 55+760+834, 55+760+835, 55+781+786, 55+781+797, 55+781+834, 55+781+835, 55+786+797, 55+786+834, 55+786+835, 55+797+834, 55+797+835, 55+834+835, 56+60+63, 56+60+79, 56+60+87, 56+60+192, 56+60+302, 56+60+387, 56+60+388, 56+60+390, 56+60+403, 56+60+408, 56+60+410, 56+60+416, 56+60+448, 56+60+451, 56+60+471, 56+60+472, 56+60+507, 56+60+512, 56+60+515, 56+60+538, 56+60+598, 56+60+602, 56+60+605, 56+60+609, 56+60+676, 56+60+694, 56+60+698, 56+60+699, 56+60+711, 56+60+754, 56+60+760, 56+60+781, 56+60+786, 56+60+797, 56+60+834, 56+60+835, 56+63+79, 56+63+87, 56+63+192, 56+63+302, 56+63+387, 56+63+388, 56+63+390, 56+63+403, 56+63+408, 56+63+410, 56+63+416, 56+63+448, 56+63+451, 56+63+471, 56+63+472, 56+63+507, 56+63+512, 56+63+515, 56+63+538, 56+63+598, 56+63+602, 56+63+605, 56+63+609, 56+63+676, 56+63+694, 56+63+698, 56+63+699, 56+63+711, 56+63+754, 56+63+760, 56+63+781, 56+63+786, 56+63+797, 56+63+834, 56+63+835, 56+79+87, 56+79+192, 56+79+302, 56+79+387, 56+79+388, 56+79+390, 56+79+403, 56+79+408, 56+79+410, 56+79+416, 56+79+448, 56+79+451, 56+79+471, 56+79+472, 56+79+507, 56+79+512, 56+79+515, 56+79+538, 56+79+598, 56+79+602, 56+79+605, 56+79+609, 56+79+676, 56+79+694, 56+79+698, 56+79+699, 56+79+711, 56+79+754, 56+79+760, 56+79+781, 56+79+786, 56+79+797, 56+79+834, 56+79+835, 56+87+192, 56+87+302, 56+87+387, 56+87+388, 56+87+390, 56+87+403, 56+87+408, 56+87+410, 56+87+416, 56+87+448, 56+87+451, 56+87+471, 56+87+472, 56+87+507, 56+87+512, 56+87+515, 56+87+538, 56+87+598, 56+87+602, 56+87+605, 56+87+609, 56+87+676, 56+87+694, 56+87+698, 56+87+699, 56+87+711, 56+87+754, 56+87+760, 56+87+781, 56+87+786, 56+87+797, 56+87+834, 56+87+835, 56+192+302, 56+192+387, 56+192+388, 56+192+390, 56+192+403, 56+192+408, 56+192+410, 56+192+416, 56+192+448, 56+192+451, 56+192+471, 56+192+472, 56+192+507, 56+192+512, 56+192+515, 56+192+538, 56+192+598, 56+192+602, 56+192+605, 56+192+609, 56+192+676, 56+192+694, 56+192+698, 56+192+699, 56+192+711, 56+192+754, 56+192+760, 56+192+781, 56+192+786, 56+192+797, 56+192+834, 56+192+835, 56+302+387, 56+302+388, 56+302+390, 56+302+403, 56+302+408, 56+302+410, 56+302+416, 56+302+448, 56+302+451, 56+302+471, 56+302+472, 56+302+507, 56+302+512, 56+302+515, 56+302+538, 56+302+598, 56+302+602, 56+302+605, 56+302+609, 56+302+676, 56+302+694, 56+302+698, 56+302+699, 56+302+711, 56+302+754, 56+302+760, 56+302+781, 56+302+786, 56+302+797, 56+302+834, 56+302+835, 56+387+388, 56+387+390, 56+387+403, 56+387+408, 56+387+410, 56+387+416, 56+387+448, 56+387+451, 56+387+471, 56+387+472, 56+387+507, 56+387+512, 56+387+515, 56+387+538, 56+387+598, 56+387+602, 56+387+605, 56+387+609, 56+387+676, 56+387+694, 56+387+698, 56+387+699, 56+387+711, 56+387+754, 56+387+760, 56+387+781, 56+387+786, 56+387+797, 56+387+834, 56+387+835, 56+388+390, 56+388+403, 56+388+408, 56+388+410, 56+388+416, 56+388+448, 56+388+451, 56+388+471, 56+388+472, 56+388+507, 56+388+512, 56+388+515, 56+388+538, 56+388+598, 56+388+602, 56+388+605, 56+388+609, 56+388+676, 56+388+694, 56+388+698, 56+388+699, 56+388+711, 56+388+754, 56+388+760, 56+388+781, 56+388+786, 56+388+797, 56+388+834, 56+388+835, 56+390+403, 56+390+408, 56+390+410, 56+390+416, 56+390+448, 56+390+451, 56+390+471, 56+390+472, 56+390+507, 56+390+512, 56+390+515, 56+390+538, 56+390+598, 56+390+602, 56+390+605, 56+390+609, 56+390+676, 56+390+694, 56+390+698, 56+390+699, 56+390+711, 56+390+754, 56+390+760, 56+390+781, 56+390+786, 56+390+797, 56+390+834, 56+390+835, 56+403+408, 56+403+410, 56+403+416, 56+403+448, 56+403+451, 56+403+471, 56+403+472, 56+403+507, 56+403+512, 56+403+515, 56+403+538, 56+403+598, 56+403+602, 56+403+605, 56+403+609, 56+403+676, 56+403+694, 56+403+698, 56+403+699, 56+403+711, 56+403+754, 56+403+760, 56+403+781, 56+403+786, 56+403+797, 56+403+834, 56+403+835, 56+408+410, 56+408+416, 56+408+448, 56+408+451, 56+408+471, 56+408+472, 56+408+507, 56+408+512, 56+408+515, 56+408+538, 56+408+598, 56+408+602, 56+408+605, 56+408+609, 56+408+676, 56+408+694, 56+408+698, 56+408+699, 56+408+711, 56+408+754, 56+408+760, 56+408+781, 56+408+786, 56+408+797, 56+408+834, 56+408+835, 56+410+416, 56+410+448, 56+410+451, 56+410+471, 56+410+472, 56+410+507, 56+410+512, 56+410+515, 56+410+538, 56+410+598, 56+410+602, 56+410+605, 56+410+609, 56+410+676, 56+410+694, 56+410+698, 56+410+699, 56+410+711, 56+410+754, 56+410+760, 56+410+781, 56+410+786, 56+410+797, 56+410+834, 56+410+835, 56+416+448, 56+416+451, 56+416+471, 56+416+472, 56+416+507, 56+416+512, 56+416+515, 56+416+538, 56+416+598, 56+416+602, 56+416+605, 56+416+609, 56+416+676, 56+416+694, 56+416+698, 56+416+699, 56+416+711, 56+416+754, 56+416+760, 56+416+781, 56+416+786, 56+416+797, 56+416+834, 56+416+835, 56+448+451, 56+448+471, 56+448+472, 56+448+507, 56+448+512, 56+448+515, 56+448+538, 56+448+598, 56+448+602, 56+448+605, 56+448+609, 56+448+676, 56+448+694, 56+448+698, 56+448+699, 56+448+711, 56+448+754, 56+448+760, 56+448+781, 56+448+786, 56+448+797, 56+448+834, 56+448+835, 56+451+471, 56+451+472, 56+451+507, 56+451+512, 56+451+515, 56+451+538, 56+451+598, 56+451+602, 56+451+605, 56+451+609, 56+451+676, 56+451+694, 56+451+698, 56+451+699, 56+451+711, 56+451+754, 56+451+760, 56+451+781, 56+451+786, 56+451+797, 56+451+834, 56+451+835, 56+471+472, 56+471+507, 56+471+512, 56+471+515, 56+471+538, 56+471+598, 56+471+602, 56+471+605, 56+471+609, 56+471+676, 56+471+694, 56+471+698, 56+471+699,6+471+711, 56+471+754, 56+471+760, 56+471+781, 56+471+786, 56+471+797, 56+471+834, 56+471+835, 56+472+507, 56+472+512, 56+472+515, 56+472+538, 56+472+598, 56+472+602, 56+472+605, 56+472+609, 56+472+676, 56+472+694, 56+472+698, 56+472+699, 56+472+711, 56+472+754, 56+472+760, 56+472+781, 56+472+786, 56+472+797, 56+472+834, 56+472+835, 56+507+512, 56+507+515, 56+507+538, 56+507+598, 56+507+602, 56+507+605, 56+507+609, 56+507+676, 56+507+694, 56+507+698, 56+507+699, 56+507+711, 56+507+754, 56+507+760, 56+507+781, 56+507+786, 56+507+797, 56+507+834, 56+507+835, 56+512+515, 56+512+538, 56+512+598, 56+512+602, 56+512+605, 56+512+609, 56+512+676, 56+512+694, 56+512+698, 56+512+699, 56+512+711, 56+512+754, 56+512+760, 56+512+781, 56+512+786, 56+512+797, 56+512+834, 56+512+835, 56+515+538, 56+515+598, 56+515+602, 56+515+605, 56+515+609, 56+515+676, 56+515+694, 56+515+698, 56+515+699, 56+515+711, 56+515+754, 56+515+760, 56+515+781, 56+515+786, 56+515+797, 56+515+834, 56+515+835, 56+538+598, 56+538+602, 56+538+605, 56+538+609, 56+538+676, 56+538+694, 56+538+698, 56+538+699, 56+538+711, 56+538+754, 56+538+760, 56+538+781, 56+538+786, 56+538+797, 56+538+834, 56+538+835, 56+598+602, 56+598+605, 56+598+609, 56+598+676, 56+598+694, 56+598+698, 56+598+699, 56+598+711, 56+598+754, 56+598+760, 56+598+781, 56+598+786, 56+598+797, 56+598+834, 56+598+835, 56+602+605, 56+602+609, 56+602+676, 56+602+694, 56+602+698, 56+602+699, 56+602+711, 56+602+754, 56+602+760, 56+602+781, 56+602+786, 56+602+797, 56+602+834, 56+602+835, 56+605+609, 56+605+676, 56+605+694, 56+605+698, 56+605+699, 56+605+711, 56+605+754, 56+605+760, 56+605+781, 56+605+786, 56+605+797, 56+605+834, 56+605+835, 56+609+676, 56+609+694, 56+609+698, 56+609+699, 56+609+711, 56+609+754, 56+609+760, 56+609+781, 56+609+786, 56+609+797, 56+609+834, 56+609+835, 56+676+694, 56+676+698, 56+676+699, 56+676+711, 56+676+754, 56+676+760, 56+676+781, 56+676+786, 56+676+797, 56+676+834, 56+676+835, 56+694+698, 56+694+699, 56+694+711, 56+694+754, 56+694+760, 56+694+781, 56+694+786, 56+694+797, 56+694+834, 56+694+835, 56+698+699, 56+698+711, 56+698+754, 56+698+760, 56+698+781, 56+698+786, 56+698+797, 56+698+834, 56+698+835, 56+699+711, 56+699+754, 56+699+760, 56+699+781, 56+699+786, 56+699+797, 56+699+834, 56+699+835, 56+711+754, 56+711+760, 56+711+781, 56+711+786, 56+711+797, 56+711+834, 56+711+835, 56+754+760, 56+754+781, 56+754+786, 56+754+797, 56+754+834, 56+754+835, 56+760+781, 56+760+786, 56+760+797, 56+760+834, 56+760+835, 56+781+786, 56+781+797, 56+781+834, 56+781+835, 56+786+797, 56+786+834, 56+786+835, 56+797+834, 56+797+835, 56+834+835, 60+63+79, 60+63+87, 60+63+192, 60+63+302, 60+63+387, 60+63+388, 60+63+390, 60+63+403, 60+63+408, 60+63+410, 60+63+416, 60+63+448, 60+63+451, 60+63+471, 60+63+472, 60+63+507, 60+63+512, 60+63+515, 60+63+538, 60+63+598, 60+63+602, 60+63+605, 60+63+609, 60+63+676, 60+63+694, 60+63+698, 60+63+699, 60+63+711, 60+63+754, 60+63+760, 60+63+781, 60+63+786, 60+63+797, 60+63+834, 60+63+835, 60+79+87, 60+79+192, 60+79+302, 60+79+387, 60+79+388, 60+79+390, 60+79+403, 60+79+408, 60+79+410, 60+79+416, 60+79+448, 60+79+451, 60+79+471, 60+79+472, 60+79+507, 60+79+512, 60+79+515, 60+79+538, 60+79+598, 60+79+602, 60+79+605, 60+79+609, 60+79+676, 60+79+694, 60+79+698, 60+79+699, 60+79+711, 60+79+754, 60+79+760, 60+79+781, 60+79+786, 60+79+797, 60+79+834, 60+79+835, 60+87+192, 60+87+302, 60+87+387, 60+87+388, 60+87+390, 60+87+403, 60+87+408, 60+87+410, 60+87+416, 60+87+448, 60+87+451, 60+87+471, 60+87+472, 60+87+507, 60+87+512, 60+87+515, 60+87+538, 60+87+598, 60+87+602, 60+87+605, 60+87+609, 60+87+676, 60+87+694, 60+87+698, 60+87+699, 60+87+711, 60+87+754, 60+87+760, 60+87+781, 60+87+786, 60+87+797, 60+87+834, 60+87+835, 60+192+302, 60+192+387, 60+192+388, 60+192+390, 60+192+403, 60+192+408, 60+192+410, 60+192+416, 60+192+448, 60+192+451, 60+192+471, 60+192+472, 60+192+507, 60+192+512, 60+192+515, 60+192+538, 60+192+598, 60+192+602, 60+192+605, 60+192+609, 60+192+676, 60+192+694, 60+192+698, 60+192+699, 60+192+711, 60+192+754, 60+192+760, 60+192+781, 60+192+786, 60+192+797, 60+192+834, 60+192+835, 60+302+387, 60+302+388, 60+302+390, 60+302+403, 60+302+408, 60+302+410, 60+302+416, 60+302+448, 60+302+451, 60+302+471, 60+302+472, 60+302+507, 60+302+512, 60+302+515, 60+302+538, 60+302+598, 60+302+602, 60+302+605, 60+302+609, 60+302+676, 60+302+694, 60+302+698, 60+302+699, 60+302+711, 60+302+754, 60+302+760, 60+302+781, 60+302+786, 60+302+797, 60+302+834, 60+302+835, 60+387+388, 60+387+390, 60+387+403, 60+387+408, 60+387+410, 60+387+416, 60+387+448, 60+387+451, 60+387+471, 60+387+472, 60+387+507, 60+387+512, 60+387+515, 60+387+538, 60+387+598, 60+387+602, 60+387+605, 60+387+609, 60+387+676, 60+387+694, 60+387+698, 60+387+699, 60+387+711, 60+387+754, 60+387+760, 60+387+781, 60+387+786, 60+387+797, 60+387+834, 60+387+835, 60+388+390, 60+388+403, 60+388+408, 60+388+410, 60+388+416, 60+388+448, 60+388+451, 60+388+471, 60+388+472, 60+388+507, 60+388+512, 60+388+515, 60+388+538, 60+388+598, 60+388+602, 60+388+605, 60+388+609, 60+388+676, 60+388+694, 60+388+698, 60+388+699, 60+388+711, 60+388+754, 60+388+760, 60+388+781, 60+388+786, 60+388+797, 60+388+834, 60+388+835, 60+390+403, 60+390+408, 60+390+410, 60+390+416, 60+390+448, 60+390+451, 60+390+471, 60+390+472, 60+390+507, 60+390+512, 60+390+515, 60+390+538, 60+390+598, 60+390+602, 60+390+605, 60+390+609, 60+390+676, 60+390+694, 60+390+698, 60+390+699, 60+390+711, 60+390+754, 60+390+760, 60+390+781, 60+390+786, 60+390+797, 60+390+834, 60+390+835, 60+403+408, 60+403+410, 60+403+416, 60+403+448, 60+403+451, 60+403+471, 60+403+472, 60+403+507, 60+403+512, 60+403+515, 60+403+538, 60+403+598, 60+403+602, 60+403+605, 60+403+609, 60+403+676, 60+403+694, 60+403+698, 60+403+699, 60+403+711, 60+403+754, 60+403+760, 60+403+781, 60+403+786, 60+403+797, 60+403+834, 60+403+835, 60+408+410, 60+408+416, 60+408+448, 60+408+451, 60+408+471, 60+408+472, 60+408+507, 60+408+512, 60+408+515, 60+408+538, 60+408+598, 60+408+602, 60+408+605, 60+408+609, 60+408+676, 60+408+694, 60+408+698, 60+408+699, 60+408+711, 60+408+754, 60+408+760, 60+408+781, 60+408+786, 60+408+797, 60+408+834, 60+408+835, 60+410+416, 60+410+448, 60+410+451, 60+410+471, 60+410+472, 60+410+507, 60+410+512, 60+410+515, 60+410+538, 60+410+598, 60+410+602, 60+410+605, 60+410+609, 60+410+676, 60+410+694, 60+410+698, 60+410+699, 60+410+711, 60+410+754, 60+410+760, 60+410+781, 60+410+786, 60+410+797, 60+410+834, 60+410+835, 60+416+448, 60+416+451, 60+416+471, 60+416+472, 60+416+507, 60+416+512, 60+416+515, 60+416+538, 60+416+598, 60+416+602, 60+416+605, 60+416+609, 60+416+676, 60+416+694, 60+416+698, 60+416+699, 60+416+711, 60+416+754, 60+416+760, 60+416+781, 60+416+786, 60+416+797, 60+416+834, 60+416+835, 60+448+451, 60+448+471, 60+448+472, 60+448+507, 60+448+512, 60+448+515, 60+448+538, 60+448+598, 60+448+602, 60+448+605, 60+448+609, 60+448+676, 60+448+694, 60+448+698, 60+448+699, 60+448+711, 60+448+754, 60+448+760, 60+448+781, 60+448+786, 60+448+797, 60+448+834, 60+448+835, 60+451+471, 60+451+472, 60+451+507, 60+451+512, 60+451+515, 60+451+538, 60+451+598, 60+451+602, 60+451+605, 60+451+609, 60+451+676, 60+451+694, 60+451+698, 60+451+699, 60+451+711, 60+451+754, 60+451+760, 60+451+781, 60+451+786, 60+451+797, 60+451+834, 60+451+835, 60+471+472, 60+471+507, 60+471+512, 60+471+515, 60+471+538, 60+471+598, 60+471+602, 60+471+605, 60+471+609, 60+471+676, 60+471+694, 60+471+698, 60+471+699, 60+471+711, 60+471+754, 60+471+760, 60+471+781, 60+471+786, 60+471+797, 60+471+834, 60+471+835, 60+472+507, 60+472+512, 60+472+515, 60+472+538, 60+472+598, 60+472+602, 60+472+605, 60+472+609, 60+472+676, 60+472+694, 60+472+698, 60+472+699, 60+472+711, 60+472+754, 60+472+760, 60+472+781, 60+472+786, 60+472+797, 60+472+834, 60+472+835, 60+507+512, 60+507+515, 60+507+538, 60+507+598, 60+507+602, 60+507+605, 60+507+609, 60+507+676, 60+507+694, 60+507+698, 60+507+699, 60+507+711, 60+507+754, 60+507+760, 60+507+781, 60+507+786, 60+507+797, 60+507+834, 60+507+835, 60+512+515, 60+512+538, 60+512+598, 60+512+602, 60+512+605, 60+512+609, 60+512+676, 60+512+694, 60+512+698, 60+512+699, 60+512+711, 60+512+754, 60+512+760, 60+512+781, 60+512+786, 60+512+797, 60+512+834, 60+512+835, 60+515+538, 60+515+598, 60+515+602, 60+515+605, 60+515+609, 60+515+676, 60+515+694, 60+515+698, 60+515+699, 60+515+711, 60+515+754, 60+515+760, 60+515+781, 60+515+786, 60+515+797, 60+515+834, 60+515+835, 60+538+598, 60+538+602, 60+538+605, 60+538+609, 60+538+676, 60+538+694, 60+538+698, 60+538+699, 60+538+711, 60+538+754, 60+538+760, 60+538+781, 60+538+786, 60+538+797, 60+538+834, 60+538+835, 60+598+602, 60+598+605, 60+598+609, 60+598+676, 60+598+694, 60+598+698, 60+598+699, 60+598+711, 60+598+754, 60+598+760, 60+598+781, 60+598+786, 60+598+797, 60+598+834, 60+598+835, 60+602+605, 60+602+609, 60+602+676, 60+602+694, 60+602+698, 60+602+699, 60+602+711, 60+602+754, 60+602+760, 60+602+781, 60+602+786, 60+602+797, 60+602+834, 60+602+835, 60+605+609, 60+605+676, 60+605+694, 60+605+698, 60+605+699, 60+605+711, 60+605+754, 60+605+760, 60+605+781, 60+605+786, 60+605+797, 60+605+834, 60+605+835, 60+609+676, 60+609+694, 60+609+698, 60+609+699, 60+609+711, 60+609+754, 60+609+760, 60+609+781, 60+609+786, 60+609+797, 60+609+834, 60+609+835, 60+676+694, 60+676+698, 60+676+699, 60+676+711, 60+676+754, 60+676+760, 60+676+781, 60+676+786, 60+676+797, 60+676+834, 60+676+835, 60+694+698, 60+694+699, 60+694+711, 60+694+754, 60+694+760, 60+694+781, 60+694+786, 60+694+797, 60+694+834, 60+694+835, 60+698+699, 60+698+711, 60+698+754, 60+698+760, 60+698+781, 60+698+786, 60+698+797, 60+698+834, 60+698+835, 60+699+711, 60+699+754, 60+699+760, 60+699+781, 60+699+786, 60+699+797, 60+699+834, 60+699+835, 60+711+754, 60+711+760, 60+711+781, 60+711+786, 60+711+797, 60+711+834, 60+711+835, 60+754+760, 60+754+781, 60+754+786, 60+754+797, 60+754+834, 60+754+835, 60+760+781, 60+760+786, 60+760+797, 60+760+834, 60+760+835, 60+781+786, 60+781+797, 60+781+834, 60+781+835, 60+786+797, 60+786+834, 60+786+835, 60+797+834, 60+797+835, 60+834+835, 63+79+87, 63+79+192, 63+79+302, 63+79+387, 63+79+388, 63+79+390, 63+79+403, 63+79+408, 63+79+410, 63+79+416, 63+79+448, 63+79+451, 63+79+471, 63+79+472, 63+79+507, 63+79+512, 63+79+515, 63+79+538, 63+79+598, 63+79+602, 63+79+605, 63+79+609, 63+79+676, 63+79+694, 63+79+698, 63+79+699, 63+79+711, 63+79+754, 63+79+760, 63+79+781, 63+79+786, 63+79+797, 63+79+834, 63+79+835, 63+87+192, 63+87+302, 63+87+387, 63+87+388, 63+87+390, 63+87+403, 63+87+408, 63+87+410, 63+87+416, 63+87+448, 63+87+451, 63+87+471, 63+87+472, 63+87+507, 63+87+512, 63+87+515, 63+87+538, 63+87+598, 63+87+602, 63+87+605, 63+87+609, 63+87+676, 63+87+694, 63+87+698, 63+87+699, 63+87+711, 63+87+754, 63+87+760, 63+87+781, 63+87+786, 63+87+797, 63+87+834, 63+87+835, 63+192+302, 63+192+387, 63+192+388, 63+192+390, 63+192+403, 63+192+408, 63+192+410, 63+192+416, 63+192+448, 63+192+451, 63+192+471, 63+192+472, 63+192+507, 63+192+512, 63+192+515, 63+192+538, 63+192+598, 63+192+602, 63+192+605, 63+192+609, 63+192+676, 63+192+694, 63+192+698, 63+192+699, 63+192+711, 63+192+754, 63+192+760, 63+192+781, 63+192+786, 63+192+797, 63+192+834, 63+192+835, 63+302+387, 63+302+388, 63+302+390, 63+302+403, 63+302+408, 63+302+410, 63+302+416, 63+302+448, 63+302+451, 63+302+471, 63+302+472, 63+302+507, 63+302+512, 63+302+515, 63+302+538, 63+302+598, 63+302+602, 63+302+605 63+302+609, 63+302+676, 63+302+694, 63+302+698, 63+302+699, 63+302+711, 63+302+754, 63+302+760, 63+302+781, 63+302+786, 63+302+797, 63+302+834, 63+302+835, 63+387+388, 63+387+390, 63+387+403, 63+387+408, 63+387+410, 63+387+416, 63+387+448, 63+387+451, 63+387+471, 63+387+472, 63+387+507, 63+387+512, 63+387+515, 63+387+538, 63+387+598, 63+387+602, 63+387+605, 63+387+609, 63+387+676, 63+387+694, 63+387+698, 63+387+699, 63+387+711, 63+387+754, 63+387+760, 63+387+781, 63+387+786, 63+387+797, 63+387+834, 63+387+835, 63+388+390, 63+388+403, 63+388+408, 63+388+410, 63+388+416, 63+388+448, 63+388+451, 63+388+471, 63+388+472, 63+388+507, 63+388+512, 63+388+515, 63+388+538, 63+388+598, 63+388+602, 63+388+605, 63+388+609, 63+388+676, 63+388+694, 63+388+698, 63+388+699, 63+388+711, 63+388+754, 63+388+760, 63+388+781, 63+388+786, 63+388+797, 63+388+834, 63+388+835, 63+390+403, 63+390+408, 63+390+410, 63+390+416, 63+390+448, 63+390+451, 63+390+471, 63+390+472, 63+390+507, 63+390+512, 63+390+515, 63+390+538, 63+390+598, 63+390+602, 63+390+605, 63+390+609, 63+390+676, 63+390+694, 63+390+698, 63+390+699, 63+390+711, 63+390+754, 63+390+760, 63+390+781, 63+390+786, 63+390+797, 63+390+834, 63+390+835, 63+403+408, 63+403+410, 63+403+416, 63+403+448, 63+403+451, 63+403+471, 63+403+472, 63+403+507, 63+403+512, 63+403+515, 63+403+538, 63+403+598, 63+403+602, 63+403+605, 63+403+609, 63+403+676, 63+403+694, 63+403+698, 63+403+699, 63+403+711, 63+403+754, 63+403+760, 63+403+781, 63+403+786, 63+403+797, 63+403+834, 63+403+835, 63+408+410, 63+408+416, 63+408+448, 63+408+451, 63+408+471, 63+408+472, 63+408+507, 63+408+512, 63+408+515, 63+408+538, 63+408+598, 63+408+602, 63+408+605, 63+408+609, 63+408+676, 63+408+694, 63+408+698, 63+408+699, 63+408+711, 63+408+754, 63+408+760, 63+408+781, 63+408+786, 63+408+797, 63+408+834, 63+408+835, 63+410+416, 63+410+448, 63+410+451, 63+410+471, 63+410+472, 63+410+507, 63+410+512, 63+410+515, 63+410+538, 63+410+598, 63+410+602, 63+410+605, 63+410+609, 63+410+676, 63+410+694, 63+410+698, 63+410+699, 63+410+711, 63+410+754, 63+410+760, 63+410+781, 63+410+786, 63+410+797, 63+410+834, 63+410+835, 63+416+448, 63+416+451, 63+416+471, 63+416+472, 63+416+507, 63+416+512, 63+416+515, 63+416+538, 63+416+598, 63+416+602, 63+416+605, 63+416+609, 63+416+676, 63+416+694, 63+416+698, 63+416+699, 63+416+711, 63+416+754, 63+416+760, 63+416+781, 63+416+786, 63+416+797, 63+416+834, 63+416+835, 63+448+451, 63+448+471, 63+448+472, 63+448+507, 63+448+512, 63+448+515, 63+448+538, 63+448+598, 63+448+602, 63+448+605, 63+448+609, 63+448+676, 63+448+694, 63+448+698, 63+448+699, 63+448+711, 63+448+754, 63+448+760, 63+448+781, 63+448+786, 63+448+797, 63+448+834, 63+448+835, 63+451+471, 63+451+472, 63+451+507, 63+451+512, 63+451+515, 63+451+538, 63+451+598, 63+451+602, 63+451+605, 63+451+609, 63+451+676, 63+451+694, 63+451+698, 63+451+699, 63+451+711, 63+451+754, 63+451+760, 63+451+781, 63+451+786, 63+451+797, 63+451+834, 63+451+835, 63+471+472, 63+471+507, 63+471+512, 63+471+515, 63+471+538, 63+471+598, 63+471+602, 63+471+605, 63+471+609, 63+471+676, 63+471+694, 63+471+698, 63+471+699, 63+471+711, 63+471+754, 63+471+760, 63+471+781, 63+471+786, 63+471+797, 63+471+834, 63+471+835, 63+472+507, 63+472+512, 63+472+515, 63+472+538, 63+472+598, 63+472+602, 63+472+605, 63+472+609, 63+472+676, 63+472+694, 63+472+698, 63+472+699, 63+472+711, 63+472+754, 63+472+760, 63+472+781, 63+472+786, 63+472+797, 63+472+834, 63+472+835, 63+507+512, 63+507+515, 63+507+538, 63+507+598, 63+507+602, 63+507+605, 63+507+609, 63+507+676, 63+507+694, 63+507+698, 63+507+699, 63+507+711, 63+507+754, 63+507+760, 63+507+781, 63+507+786, 63+507+797, 63+507+834, 63+507+835, 63+512+515, 63+512+538, 63+512+598, 63+512+602, 63+512+605, 63+512+609, 63+512+676, 63+512+694, 63+512+698, 63+512+699, 63+512+711, 63+512+754, 63+512+760, 63+512+781, 63+512+786, 63+512+797, 63+512+834, 63+512+835, 63+515+538, 63+515+598, 63+515+602, 63+515+605, 63+515+609, 63+515+676, 63+515+694, 63+515+698, 63+515+699, 63+515+711, 63+515+754, 63+515+760, 63+515+781, 63+515+786, 63+515+797, 63+515+834, 63+515+835, 63+538+598, 63+538+602, 63+538+605, 63+538+609, 63+538+676, 63+538+694, 63+538+698, 63+538+699, 63+538+711, 63+538+754, 63+538+760, 63+538+781, 63+538+786, 63+538+797, 63+538+834, 63+538+835, 63+598+602, 63+598+605, 63+598+609, 63+598+676, 63+598+694, 63+598+698, 63+598+699, 63+598+711, 63+598+754, 63+598+760, 63+598+781, 63+598+786, 63+598+797, 63+598+834, 63+598+835, 63+602+605, 63+602+609, 63+602+676, 63+602+694, 63+602+698, 63+602+699, 63+602+711, 63+602+754, 63+602+760, 63+602+781, 63+602+786, 63+602+797, 63+602+834, 63+602+835, 63+605+609, 63+605+676, 63+605+694, 63+605+698, 63+605+699, 63+605+711, 63+605+754, 63+605+760, 63+605+781, 63+605+786, 63+605+797, 63+605+834, 63+605+835, 63+609+676, 63+609+694, 63+609+698, 63+609+699, 63+609+711, 63+609+754, 63+609+760, 63+609+781, 63+609+786, 63+609+797, 63+609+834, 63+609+835, 63+676+694, 63+676+698, 63+676+699, 63+676+711, 63+676+754, 63+676+760, 63+676+781, 63+676+786, 63+676+797, 63+676+834, 63+676+835, 63+694+698, 63+694+699, 63+694+711, 63+694+754, 63+694+760, 63+694+781, 63+694+786, 63+694+797, 63+694+834, 63+694+835, 63+698+699, 63+698+711, 63+698+754, 63+698+760, 63+698+781, 63+698+786, 63+698+797, 63+698+834, 63+698+835, 63+699+711, 63+699+754, 63+699+760, 63+699+781, 63+699+786, 63+699+797, 63+699+834, 63+699+835, 63+711+754, 63+711+760, 63+711+781, 63+711+786, 63+711+797, 63+711+834, 63+711+835, 63+754+760, 63+754+781, 63+754+786, 63+754+797, 63+754+834, 63+754+835, 63+760+781, 63+760+786, 63+760+797, 63+760+834, 63+760+835, 63+781+786, 63+781+797, 63+781+834, 63+781+835, 63+786+797, 63+786+834, 63+786+835, 63+797+834, 63+797+835, 63+834+835, 79+87+192, 79+87+302, 79+87+387, 79+87+388, 79+87+390, 79+87+403, 79+87+408, 79+87+410, 79+87+416, 79+87+448, 79+87+451, 79+87+471, 79+87+472, 79+87+507, 79+87+512, 79+87+515, 79+87+538, 79+87+598, 79+87+602, 79+87+605, 79+87+609, 79+87+676, 79+87+694, 79+87+698, 79+87+699, 79+87+711, 79+87+754, 79+87+760, 79+87+781, 79+87+786, 79+87+797, 79+87+834, 79+87+835, 79+192+302, 79+192+387, 79+192+388, 79+192+390, 79+192+403, 79+192+408, 79+192+410, 79+192+416, 79+192+448, 79+192+451, 79+192+471, 79+192+472, 79+192+507, 79+192+512, 79+192+515, 79+192+538, 79+192+598, 79+192+602, 79+192+605, 79+192+609, 79+192+676, 79+192+694, 79+192+698, 79+192+699, 79+192+711, 79+192+754, 79+192+760, 79+192+781, 79+192+786, 79+192+797, 79+192+834, 79+192+835, 79+302+387, 79+302+388, 79+302+390, 79+302+403, 79+302+408, 79+302+410, 79+302+416, 79+302+448, 79+302+451, 79+302+471, 79+302+472, 79+302+507, 79+302+512, 79+302+515, 79+302+538, 79+302+598, 79+302+602, 79+302+605, 79+302+609, 79+302+676, 79+302+694, 79+302+698, 79+302+699, 79+302+711, 79+302+754, 79+302+760, 79+302+781, 79+302+786, 79+302+797, 79+302+834, 79+302+835, 79+387+388, 79+387+390, 79+387+403, 79+387+408, 79+387+410, 79+387+416, 79+387+448, 79+387+451, 79+387+471, 79+387+472, 79+387+507, 79+387+512, 79+387+515, 79+387+538, 79+387+598, 79+387+602, 79+387+605, 79+387+609, 79+387+676, 79+387+694, 79+387+698, 79+387+699, 79+387+711, 79+387+754, 79+387+760, 79+387+781, 79+387+786, 79+387+797, 79+387+834, 79+387+835, 79+388+390, 79+388+403, 79+388+408, 79+388+410, 79+388+416, 79+388+448, 79+388+451, 79+388+471, 79+388+472, 79+388+507, 79+388+512, 79+388+515, 79+388+538, 79+388+598, 79+388+602, 79+388+605, 79+388+609, 79+388+676, 79+388+694, 79+388+698, 79+388+699, 79+388+711, 79+388+754, 79+388+760, 79+388+781, 79+388+786, 79+388+797, 79+388+834, 79+388+835, 79+390+403, 79+390+408, 79+390+410, 79+390+416, 79+390+448, 79+390+451, 79+390+471, 79+390+472, 79+390+507, 79+390+512, 79+390+515, 79+390+538, 79+390+598, 79+390+602, 79+390+605, 79+390+609, 79+390+676, 79+390+694, 79+390+698, 79+390+699, 79+390+711, 79+390+754, 79+390+760, 79+390+781, 79+390+786, 79+390+797, 79+390+834, 79+390+835, 79+403+408, 79+403+410, 79+403+416, 79+403+448, 79+403+451, 79+403+471, 79+403+472, 79+403+507, 79+403+512, 79+403+515, 79+403+538, 79+403+598, 79+403+602, 79+403+605, 79+403+609, 79+403+676, 79+403+694, 79+403+698, 79+403+699, 79+403+711, 79+403+754, 79+403+760, 79+403+781, 79+403+786, 79+403+797, 79+403+834, 79+403+835, 79+408+410, 79+408+416, 79+408+448, 79+408+451, 79+408+471, 79+408+472, 79+408+507, 79+408+512, 79+408+515, 79+408+538, 79+408+598, 79+408+602, 79+408+605, 79+408+609, 79+408+676, 79+408+694, 79+408+698, 79+408+699, 79+408+711, 79+408+754, 79+408+760, 79+408+781, 79+408+786, 79+408+797, 79+408+834, 79+408+835, 79+410+416, 79+410+448, 79+410+451, 79+410+471, 79+410+472, 79+410+507, 79+410+512, 79+410+515, 79+410+538, 79+410+598, 79+410+602, 79+410+605, 79+410+609, 79+410+676, 79+410+694, 79+410+698, 79+410+699, 79+410+711, 79+410+754, 79+410+760, 79+410+781, 79+410+786, 79+410+797, 79+410+834, 79+410+835,9+416+448, 79+416+451, 79+416+471, 79+416+472, 79+416+507, 79+416+512, 79+416+515, 79+416+538, 79+416+598, 79+416+602, 79+416+605, 79+416+609, 79+416+676, 79+416+694, 79+416+698, 79+416+699, 79+416+711, 79+416+754, 79+416+760, 79+416+781, 79+416+786, 79+416+797, 79+416+834, 79+416+835, 79+448+451, 79+448+471, 79+448+472, 79+448+507, 79+448+512, 79+448+515, 79+448+538, 79+448+598, 79+448+602, 79+448+605, 79+448+609, 79+448+676, 79+448+694, 79+448+698, 79+448+699, 79+448+711, 79+448+754, 79+448+760, 79+448+781, 79+448+786, 79+448+797, 79+448+834, 79+448+835, 79+451+471, 79+451+472, 79+451+507, 79+451+512, 79+451+515, 79+451+538, 79+451+598, 79+451+602, 79+451+605, 79+451+609, 79+451+676, 79+451+694, 79+451+698, 79+451+699, 79+451+711, 79+451+754, 79+451+760, 79+451+781, 79+451+786, 79+451+797, 79+451+834, 79+451+835, 79+471+472, 79+471+507, 79+471+512, 79+471+515, 79+471+538, 79+471+598, 79+471+602, 79+471+605, 79+471+609, 79+471+676, 79+471+694, 79+471+698, 79+471+699, 79+471+711, 79+471+754, 79+471+760, 79+471+781, 79+471+786, 79+471+797, 79+471+834, 79+471+835, 79+472+507, 79+472+512, 79+472+515, 79+472+538, 79+472+598, 79+472+602, 79+472+605, 79+472+609, 79+472+676, 79+472+694, 79+472+698, 79+472+699, 79+472+711, 79+472+754, 79+472+760, 79+472+781, 79+472+786, 79+472+797, 79+472+834, 79+472+835, 79+507+512, 79+507+515, 79+507+538, 79+507+598, 79+507+602, 79+507+605, 79+507+609, 79+507+676, 79+507+694, 79+507+698, 79+507+699, 79+507+711, 79+507+754, 79+507+760, 79+507+781, 79+507+786, 79+507+797, 79+507+834, 79+507+835, 79+512+515, 79+512+538, 79+512+598, 79+512+602, 79+512+605, 79+512+609, 79+512+676, 79+512+694, 79+512+698, 79+512+699, 79+512+711, 79+512+754, 79+512+760, 79+512+781, 79+512+786, 79+512+797, 79+512+834, 79+512+835, 79+515+538, 79+515+598, 79+515+602, 79+515+605, 79+515+609, 79+515+676, 79+515+694, 79+515+698, 79+515+699, 79+515+711, 79+515+754, 79+515+760, 79+515+781, 79+515+786, 79+515+797, 79+515+834, 79+515+835, 79+538+598, 79+538+602, 79+538+605, 79+538+609, 79+538+676, 79+538+694, 79+538+698, 79+538+699, 79+538+711, 79+538+754, 79+538+760, 79+538+781, 79+538+786, 79+538+797, 79+538+834, 79+538+835, 79+598+602, 79+598+605, 79+598+609, 79+598+676, 79+598+694, 79+598+698, 79+598+699, 79+598+711, 79+598+754, 79+598+760, 79+598+781, 79+598+786, 79+598+797, 79+598+834, 79+598+835, 79+602+605, 79+602+609, 79+602+676, 79+602+694, 79+602+698, 79+602+699, 79+602+711, 79+602+754, 79+602+760, 79+602+781, 79+602+786, 79+602+797, 79+602+834, 79+602+835, 79+605+609, 79+605+676, 79+605+694, 79+605+698, 79+605+699, 79+605+711, 79+605+754, 79+605+760, 79+605+781, 79+605+786, 79+605+797, 79+605+834, 79+605+835, 79+609+676, 79+609+694, 79+609+698, 79+609+699,9+609+711, 79+609+754, 79+609+760, 79+609+781, 79+609+786, 79+609+797, 79+609+834, 79+609+835, 79+676+694, 79+676+698, 79+676+699, 79+676+711, 79+676+754, 79+676+760, 79+676+781, 79+676+786, 79+676+797, 79+676+834, 79+676+835, 79+694+698, 79+694+699, 79+694+711, 79+694+754, 79+694+760, 79+694+781, 79+694+786, 79+694+797, 79+694+834, 79+694+835, 79+698+699, 79+698+711, 79+698+754, 79+698+760, 79+698+781, 79+698+786, 79+698+797, 79+698+834, 79+698+835, 79+699+711, 79+699+754, 79+699+760, 79+699+781, 79+699+786, 79+699+797, 79+699+834, 79+699+835, 79+711+754, 79+711+760, 79+711+781, 79+711+786, 79+711+797, 79+711+834, 79+711+835, 79+754+760, 79+754+781, 79+754+786, 79+754+797, 79+754+834, 79+754+835, 79+760+781, 79+760+786, 79+760+797, 79+760+834, 79+760+835, 79+781+786, 79+781+797, 79+781+834, 79+781+835, 79+786+797, 79+786+834, 79+786+835, 79+797+834, 79+797+835, 79+834+835, 87+192+302, 87+192+387, 87+192+388, 87+192+390, 87+192+403, 87+192+408, 87+192+410, 87+192+416, 87+192+448, 87+192+451, 87+192+471, 87+192+472, 87+192+507, 87+192+512, 87+192+515, 87+192+538, 87+192+598, 87+192+602, 87+192+605, 87+192+609, 87+192+676, 87+192+694, 87+192+698, 87+192+699, 87+192+711, 87+192+754, 87+192+760, 87+192+781, 87+192+786, 87+192+797, 87+192+834, 87+192+835, 87+302+387, 87+302+388, 87+302+390, 87+302+403, 87+302+408, 87+302+410, 87+302+416, 87+302+448, 87+302+451, 87+302+471, 87+302+472, 87+302+507, 87+302+512, 87+302+515, 87+302+538, 87+302+598, 87+302+602, 87+302+605, 87+302+609, 87+302+676, 87+302+694, 87+302+698, 87+302+699, 87+302+711, 87+302+754, 87+302+760,7+302+781, 87+302+786, 87+302+797, 87+302+834, 87+302+835, 87+387+388, 87+387+390, 87+387+403, 87+387+408, 87+387+410, 87+387+416, 87+387+448, 87+387+451, 87+387+471, 87+387+472, 87+387+507, 87+387+512, 87+387+515, 87+387+538, 87+387+598, 87+387+602, 87+387+605, 87+387+609, 87+387+676, 87+387+694, 87+387+698, 87+387+699, 87+387+711, 87+387+754, 87+387+760, 87+387+781, 87+387+786, 87+387+797, 87+387+834, 87+387+835, 87+388+390, 87+388+403, 87+388+408, 87+388+410, 87+388+416, 87+388+448, 87+388+451, 87+388+471, 87+388+472, 87+388+507, 87+388+512, 87+388+515, 87+388+538, 87+388+598, 87+388+602, 87+388+605, 87+388+609, 87+388+676, 87+388+694, 87+388+698, 87+388+699, 87+388+711, 87+388+754, 87+388+760, 87+388+781, 87+388+786, 87+388+797, 87+388+834, 87+388+835, 87+390+403, 87+390+408, 87+390+410, 87+390+416, 87+390+448, 87+390+451, 87+390+471, 87+390+472, 87+390+507, 87+390+512, 87+390+515, 87+390+538, 87+390+598, 87+390+602, 87+390+605, 87+390+609, 87+390+676, 87+390+694, 87+390+698, 87+390+699, 87+390+711, 87+390+754, 87+390+760, 87+390+781, 87+390+786, 87+390+797, 87+390+834, 87+390+835, 87+403+408, 87+403+410, 87+403+416, 87+403+448, 87+403+451, 87+403+471, 87+403+472, 87+403+507, 87+403+512, 87+403+515, 87+403+538, 87+403+598, 87+403+602, 87+403+605, 87+403+609, 87+403+676, 87+403+694, 87+403+698, 87+403+699, 87+403+711, 87+403+754, 87+403+760, 87+403+781, 87+403+786, 87+403+797, 87+403+834, 87+403+835, 87+408+410, 87+408+416, 87+408+448, 87+408+451, 87+408+471, 87+408+472, 87+408+507, 87+408+512, 87+408+515, 87+408+538, 87+408+598, 87+408+602, 87+408+605, 87+408+609, 87+408+676, 87+408+694, 87+408+698, 87+408+699, 87+408+711, 87+408+754, 87+408+760, 87+408+781, 87+408+786, 87+408+797, 87+408+834, 87+408+835, 87+410+416, 87+410+448, 87+410+451, 87+410+471, 87+410+472, 87+410+507, 87+410+512, 87+410+515, 87+410+538, 87+410+598, 87+410+602, 87+410+605, 87+410+609, 87+410+676, 87+410+694, 87+410+698, 87+410+699, 87+410+711, 87+410+754, 87+410+760, 87+410+781, 87+410+786, 87+410+797, 87+410+834, 87+410+835, 87+416+448, 87+416+451, 87+416+471, 87+416+472, 87+416+507, 87+416+512, 87+416+515, 87+416+538, 87+416+598, 87+416+602, 87+416+605, 87+416+609, 87+416+676, 87+416+694, 87+416+698, 87+416+699, 87+416+711, 87+416+754, 87+416+760, 87+416+781, 87+416+786, 87+416+797, 87+416+834, 87+416+835, 87+448+451, 87+448+471, 87+448+472, 87+448+507, 87+448+512, 87+448+515, 87+448+538, 87+448+598, 87+448+602, 87+448+605, 87+448+609, 87+448+676, 87+448+694, 87+448+698, 87+448+699, 87+448+711, 87+448+754, 87+448+760, 87+448+781, 87+448+786, 87+448+797, 87+448+834, 87+448+835, 87+451+471, 87+451+472, 87+451+507, 87+451+512, 87+451+515, 87+451+538, 87+451+598, 87+451+602, 87+451+605, 87+451+609, 87+451+676, 87+451+694, 87+451+698, 87+451+699, 87+451+711, 87+451+754, 87+451+760, 87+451+781, 87+451+786, 87+451+797, 87+451+834, 87+451+835, 87+471+472, 87+471+507, 87+471+512, 87+471+515, 87+471+538, 87+471+598, 87+471+602, 87+471+605, 87+471+609, 87+471+676, 87+471+694, 87+471+698, 87+471+699, 87+471+711, 87+471+754, 87+471+760, 87+471+781, 87+471+786, 87+471+797, 87+471+834, 87+471+835, 87+472+507, 87+472+512, 87+472+515, 87+472+538, 87+472+598, 87+472+602, 87+472+605, 87+472+609, 87+472+676, 87+472+694, 87+472+698, 87+472+699, 87+472+711, 87+472+754, 87+472+760, 87+472+781, 87+472+786, 87+472+797, 87+472+834, 87+472+835, 87+507+512, 87+507+515, 87+507+538, 87+507+598, 87+507+602, 87+507+605, 87+507+609, 87+507+676, 87+507+694, 87+507+698, 87+507+699, 87+507+711, 87+507+754, 87+507+760, 87+507+781, 87+507+786, 87+507+797, 87+507+834, 87+507+835, 87+512+515, 87+512+538, 87+512+598, 87+512+602, 87+512+605, 87+512+609, 87+512+676, 87+512+694, 87+512+698, 87+512+699, 87+512+711, 87+512+754, 87+512+760, 87+512+781, 87+512+786, 87+512+797, 87+512+834, 87+512+835, 87+515+538, 87+515+598, 87+515+602, 87+515+605, 87+515+609, 87+515+676, 87+515+694, 87+515+698, 87+515+699, 87+515+711, 87+515+754, 87+515+760, 87+515+781, 87+515+786, 87+515+797, 87+515+834, 87+515+835, 87+538+598, 87+538+602, 87+538+605, 87+538+609, 87+538+676, 87+538+694, 87+538+698, 87+538+699, 87+538+711, 87+538+754, 87+538+760, 87+538+781, 87+538+786, 87+538+797, 87+538+834, 87+538+835, 87+598+602, 87+598+605, 87+598+609, 87+598+676, 87+598+694, 87+598+698, 87+598+699, 87+598+711, 87+598+754, 87+598+760, 87+598+781, 87+598+786, 87+598+797, 87+598+834, 87+598+835, 87+602+605, 87+602+609, 87+602+676, 87+602+694, 87+602+698, 87+602+699, 87+602+711, 87+602+754, 87+602+760, 87+602+781, 87+602+786, 87+602+797, 87+602+834, 87+602+835, 87+605+609, 87+605+676, 87+605+694, 87+605+698, 87+605+699, 87+605+711, 87+605+754, 87+605+760, 87+605+781, 87+605+786, 87+605+797, 87+605+834, 87+605+835, 87+609+676, 87+609+694, 87+609+698, 87+609+699, 87+609+711, 87+609+754, 87+609+760, 87+609+781, 87+609+786, 87+609+797, 87+609+834, 87+609+835, 87+676+694, 87+676+698, 87+676+699, 87+676+711, 87+676+754, 87+676+760, 87+676+781, 87+676+786, 87+676+797, 87+676+834, 87+676+835, 87+694+698, 87+694+699, 87+694+711, 87+694+754, 87+694+760, 87+694+781, 87+694+786, 87+694+797, 87+694+834, 87+694+835, 87+698+699, 87+698+711, 87+698+754, 87+698+760, 87+698+781, 87+698+786, 87+698+797, 87+698+834, 87+698+835, 87+699+711, 87+699+754, 87+699+760, 87+699+781, 87+699+786, 87+699+797, 87+699+834, 87+699+835, 87+711+754, 87+711+760, 87+711+781, 87+711+786, 87+711+797, 87+711+834, 87+711+835, 87+754+760, 87+754+781, 87+754+786, 87+754+797, 87+754+834, 87+754+835, 87+760+781, 87+760+786, 87+760+797, 87+760+834, 87+760+835, 87+781+786, 87+781+797, 87+781+834, 87+781+835, 87+786+797, 87+786+834, 87+786+835, 87+797+834, 87+797+835, 87+834+835, 192+302+387, 192+302+388, 192+302+390, 192+302+403, 192+302+408, 192+302+410, 192+302+416, 192+302+448, 192+302+45t192+302+471, 192+302+472, 192+302+507, 192+302+512, 192+302+515, 192+302+538, 192+302+598, 192+302+602, 192+302+605, 192+302+609, 192+302+676, 192+302+694, 192+302+698, 192+302+699, 192+302+711, 192+302+754, 192+302+760, 192+302+78t192+302+786, 192+302+797, 192+302+834, 192+302+835, 192+387+388, 192+387+390, 192+387+403, 192+387+408, 192+387+410, 192+387+416, 192+387+448, 192+387+45t192+387+471, 192+387+472, 192+387+507, 192+387+512, 192+387+515, 192+387+538, 192+387+598, 192+387+602, 192+387+605, 192+387+609, 192+387+676, 192+387+694, 192+387+698, 192+387+699, 192+387+711, 192+387+754, 192+387+760, 192+387+781, 192+387+786, 192+387+797, 192+387+834, 192+387+835, 192+388+390, 192+388+403, 192+388+408, 192+388+410, 192+388+416, 192+388+448, 192+388+451, 192+388+471, 192+388+472, 192+388+507, 192+388+512, 192+388+515, 192+388+538, 192+388+598, 192+388+602, 192+388+605, 192+388+609, 192+388+676, 192+388+694, 192+388+698, 192+388+699, 192+388+711, 192+388+754, 192+388+760, 192+388+781, 192+388+786, 192+388+797, 192+388+834, 192+388+835, 192+390+403, 192+390+408, 192+390+410, 192+390+416, 192+390+448, 192+390+451, 192+390+471, 192+390+472, 192+390+507, 192+390+512, 192+390+515, 192+390+538, 192+390+598, 192+390+602, 192+390+605, 192+390+609, 192+390+676, 192+390+694, 192+390+698, 192+390+699, 192+390+711, 192+390+754, 192+390+760, 192+390+781, 192+390+786, 192+390+797, 192+390+834, 192+390+835, 192+403+408, 192+403+410, 192+403+416, 192+403+448, 192+403+451, 192+403+471, 192+403+472, 192+403+507, 192+403+512, 192+403+515, 192+403+538, 192+403+598, 192+403+602, 192+403+605, 192+403+609, 192+403+676, 192+403+694, 192+403+698, 192+403+699, 192+403+711, 192+403+754, 192+403+760, 192+403+781, 192+403+786, 192+403+797, 192+403+834, 192+403+835, 192+408+410, 192+408+416, 192+408+448, 192+408+451, 192+408+471, 192+408+472, 192+408+507, 192+408+512, 192+408+515, 192+408+538, 192+408+598, 192+408+602, 192+408+605, 192+408+609, 192+408+676, 192+408+694, 192+408+698, 192+408+699, 192+408+711, 192+408+754, 192+408+760, 192+408+781, 192+408+786, 192+408+797, 192+408+834, 192+408+835, 192+410+416, 192+410+448, 192+410+451, 192+410+471, 192+410+472, 192+410+507, 192+410+512, 192+410+515, 192+410+538, 192+410+598, 192+410+602, 192+410+605, 192+410+609, 192+410+676, 192+410+694, 192+410+698, 192+410+699, 192+410+711, 192+410+754, 192+410+760, 192+410+781, 192+410+786, 192+410+797, 192+410+834, 192+410+835, 192+416+448, 192+416+451, 192+416+471, 192+416+472, 192+416+507, 192+416+512, 192+416+515, 192+416+538, 192+416+598, 192+416+602, 192+416+605, 192+416+609, 192+416+676, 192+416+694, 192+416+698, 192+416+699, 192+416+711, 192+416+754, 192+416+760, 192+416+781, 192+416+786, 192+416+797, 192+416+834, 192+416+835, 192+448+451, 192+448+471, 192+448+472, 192+448+507, 192+448+512, 192+448+515, 192+448+538, 192+448+598, 192+448+602, 192+448+605, 192+448+609, 192+448+676, 192+448+694, 192+448+698, 192+448+699, 192+448+711, 192+448+754, 192+448+760, 192+448+781, 192+448+786, 192+448+797, 192+448+834, 192+448+835, 192+451+471, 192+451+472, 192+451+507, 192+451+512, 192+451+515, 192+451+538, 192+451+598, 192+451+602, 192+451+605, 192+451+609, 192+451+676, 192+451+694, 192+451+698, 192+451+699, 192+451+711, 192+451+754, 192+451+760, 192+451+781, 192+451+786, 192+451+797, 192+451+834, 192+451+835, 192+471+472, 192+471+507, 192+471+512, 192+471+515, 192+471+538, 192+471+598, 192+471+602, 192+471+605, 192+471+609, 192+471+676, 192+471+694, 192+471+698, 192+471+699, 192+471+711, 192+471+754, 192+471+760, 192+471+781, 192+471+786, 192+471+797, 192+471+834, 192+471+835, 192+472+507, 192+472+512, 192+472+515, 192+472+538, 192+472+598, 192+472+602, 192+472+605, 192+472+609, 192+472+676, 192+472+694, 192+472+698, 192+472+699, 192+472+711, 192+472+754, 192+472+760, 192+472+781, 192+472+786, 192+472+797, 192+472+834, 192+472+835, 192+507+512, 192+507+515, 192+507+538, 192+507+598, 192+507+602, 192+507+605, 192+507+609, 192+507+676, 192+507+694, 192+507+698, 192+507+699, 192+507+711, 192+507+754, 192+507+760, 192+507+781, 192+507+786, 192+507+797, 192+507+834, 192+507+835, 192+512+515, 192+512+538, 192+512+598, 192+512+602, 192+512+605, 192+512+609, 192+512+676, 192+512+694, 192+512+698, 192+512+699, 192+512+711, 192+512+754, 192+512+760, 192+512+781, 192+512+786, 192+512+797, 192+512+834, 192+512+835, 192+515+538, 192+515+598 192+515+602, 192+515+605, 192+515+609, 192+515+676, 192+515+694, 192+515+698, 192+515+699, 192+515+711, 192+515+754, 192+515+760, 192+515+781, 192+515+786, 192+515+797, 192+515+834, 192+515+835, 192+538+598, 192+538+602, 192+538+605, 192+538+609, 192+538+676, 192+538+694, 192+538+698, 192+538+699, 192+538+711, 192+538+754, 192+538+760, 192+538+781, 192+538+786, 192+538+797, 192+538+834, 192+538+835, 192+598+602, 192+598+605, 192+598+609, 192+598+676, 192+598+694, 192+598+698, 192+598+699, 192+598+711, 192+598+754, 192+598+760, 192+598+781, 192+598+786, 192+598+797, 192+598+834, 192+598+835, 192+602+605, 192+602+609, 192+602+676, 192+602+694, 192+602+698, 192+602+699, 192+602+711, 192+602+754, 192+602+760, 192+602+781, 192+602+786, 192+602+797, 192+602+834, 192+602+835, 192+605+609, 192+605+676, 192+605+694, 192+605+698, 192+605+699, 192+605+711, 192+605+754, 192+605+760, 192+605+781, 192+605+786, 192+605+797, 192+605+834, 192+605+835, 192+609+676, 192+609+694, 192+609+698, 192+609+699, 192+609+711, 192+609+754, 192+609+760, 192+609+781, 192+609+786, 192+609+797, 192+609+834, 192+609+835, 192+676+694, 192+676+698, 192+676+699, 192+676+711, 192+676+754, 192+676+760, 192+676+781, 192+676+786, 192+676+797, 192+676+834, 192+676+835, 192+694+698, 192+694+699, 192+694+711, 192+694+754, 192+694+760, 192+694+781, 192+694+786, 192+694+797, 192+694+834, 192+694+835, 192+698+699, 192+698+711, 192+698+754, 192+698+760, 192+698+781, 192+698+786, 192+698+797, 192+698+834, 192+698+835, 192+699+711, 192+699+754, 192+699+760, 192+699+781, 192+699+786, 192+699+797, 192+699+834, 192+699+835, 192+711+754, 192+711+760, 192+711+781, 192+711+786, 192+711+797, 192+711+834, 192+711+835, 192+754+760, 192+754+781, 192+754+786, 192+754+797, 192+754+834, 192+754+835, 192+760+781, 192+760+786, 192+760+797, 192+760+834, 192+760+835, 192+781+786, 192+781+797, 192+781+834, 192+781+835, 192+786+797, 192+786+834, 192+786+835, 192+797+834, 192+797+835, 192+834+835, 302+387+388, 302+387+390, 302+387+403, 302+387+408, 302+387+410, 302+387+416, 302+387+448, 302+387+451, 302+387+471, 302+387+472, 302+387+507, 302+387+512, 302+387+515, 302+387+538, 302+387+598, 302+387+602, 302+387+605, 302+387+609, 302+387+676, 302+387+694, 302+387+698, 302+387+699, 302+387+711, 302+387+754, 302+387+760, 302+387+781, 302+387+786, 302+387+797, 302+387+834, 302+387+835, 302+388+390, 302+388+403, 302+388+408, 302+388+410, 302+388+416, 302+388+448, 302+388+451, 302+388+471, 302+388+472, 302+388+507, 302+388+512, 302+388+515, 302+388+538, 302+388+598, 302+388+602, 302+388+605, 302+388+609, 302+388+676, 302+388+694, 302+388+698, 302+388+699, 302+388+711, 302+388+754, 302+388+760, 302+388+781, 302+388+786, 302+388+797, 302+388+834, 302+388+835, 302+390+403, 302+390+408, 302+390+410, 302+390+416, 302+390+448, 302+390+451, 302+390+471, 302+390+472, 302+390+507, 302+390+512, 302+390+515, 302+390+538, 302+390+598, 302+390+602, 302+390+605, 302+390+609, 302+390+676, 302+390+694, 302+390+698, 302+390+699, 302+390+711, 302+390+754, 302+390+760, 302+390+781, 302+390+786, 302+390+797, 302+390+834, 302+390+835, 302+403+408, 302+403+410, 302+403+416, 302+403+448, 302+403+451, 302+403+471, 302+403+472, 302+403+507, 302+403+512, 302+403+515, 302+403+538, 302+403+598, 302+403+602, 302+403+605, 302+403+609, 302+403+676, 302+403+694, 302+403+698, 302+403+699, 302+403+711, 302+403+754, 302+403+760, 302+403+781, 302+403+786, 302+403+797, 302+403+834, 302+403+835, 302+408+410, 302+408+416, 302+408+448, 302+408+451, 302+408+471, 302+408+472, 302+408+507, 302+408+512, 302+408+515, 302+408+538, 302+408+598, 302+408+602, 302+408+605, 302+408+609, 302+408+676, 302+408+694, 302+408+698, 302+408+699, 302+408+711, 302+408+754, 302+408+760, 302+408+781, 302+408+786, 302+408+797, 302+408+834, 302+408+835, 302+410+416, 302+410+448, 302+410+451, 302+410+471, 302+410+472, 302+410+507, 302+410+512, 302+410+515, 302+410+538, 302+410+598, 302+410+602, 302+410+605, 302+410+609, 302+410+676, 302+410+694, 302+410+698, 302+410+699, 302+410+711, 302+410+754, 302+410+760, 302+410+781, 302+410+786, 302+410+797, 302+410+834, 302+410+835, 302+416+448, 302+416+451, 302+416+471, 302+416+472, 302+416+507, 302+416+512, 302+416+515, 302+416+538, 302+416+598, 302+416+602, 302+416+605, 302+416+609, 302+416+676, 302+416+694, 302+416+698, 302+416+699, 302+416+711, 302+416+754, 302+416+760, 302+416+781, 302+416+786, 302+416+797, 302+416+834, 302+416+835, 302+448+451, 302+448+471, 302+448+472, 302+448+507, 302+448+512, 302+448+515, 302+448+538, 302+448+598, 302+448+602, 302+448+605, 302+448+609, 302+448+676, 302+448+694, 302+448+698, 302+448+699, 302+448+711, 302+448+754, 302+448+760, 302+448+781, 302+448+786, 302+448+797, 302+448+834, 302+448+835, 302+451+471, 302+451+472, 302+451+507, 302+451+512, 302+451+515, 302+451+538, 302+451+598, 302+451+602, 302+451+605, 302+451+609, 302+451+676, 302+451+694, 302+451+698, 302+451+699, 302+451+711, 302+451+754, 302+451+760, 302+451+781, 302+451+786, 302+451+797, 302+451+834, 302+451+835 302+471+472, 302+471+507, 302+471+512, 302+471+515, 302+471+538, 302+471+598, 302+471+602, 302+471+605, 302+471+609, 302+471+676, 302+471+694, 302+471+698, 302+471+699, 302+471+711, 302+471+754, 302+471+760, 302+471+781, 302+471+786, 302+471+797, 302+471+834, 302+471+835, 302+472+507, 302+472+512, 302+472+515, 302+472+538, 302+472+598, 302+472+602, 302+472+605, 302+472+609, 302+472+676, 302+472+694, 302+472+698, 302+472+699, 302+472+711, 302+472+754, 302+472+760, 302+472+781, 302+472+786, 302+472+797, 302+472+834, 302+472+835, 302+507+512, 302+507+515, 302+507+538, 302+507+598, 302+507+602, 302+507+605, 302+507+609, 302+507+676, 302+507+694, 302+507+698, 302+507+699, 302+507+711, 302+507+754, 302+507+760, 302+507+781, 302+507+786, 302+507+797, 302+507+834, 302+507+835, 302+512+515, 302+512+538, 302+512+598, 302+512+602, 302+512+605, 302+512+609, 302+512+676, 302+512+694, 302+512+698, 302+512+699, 302+512+711, 302+512+754, 302+512+760, 302+512+781, 302+512+786, 302+512+797, 302+512+834, 302+512+835, 302+515+538, 302+515+598, 302+515+602, 302+515+605, 302+515+609, 302+515+676, 302+515+694, 302+515+698, 302+515+699, 302+515+711, 302+515+754, 302+515+760, 302+515+781, 302+515+786, 302+515+797, 302+515+834, 302+515+835, 302+538+598, 302+538+602, 302+538+605, 302+538+609, 302+538+676, 302+538+694, 302+538+698, 302+538+699, 302+538+711, 302+538+754, 302+538+760, 302+538+781, 302+538+786, 302+538+797, 302+538+834, 302+538+835, 302+598+602, 302+598+605, 302+598+609, 302+598+676, 302+598+694, 302+598+698, 302+598+699, 302+598+711, 302+598+754, 302+598+760, 302+598+781, 302+598+786, 302+598+797, 302+598+834, 302+598+835, 302+602+605, 302+602+609, 302+602+676, 302+602+694, 302+602+698, 302+602+699, 302+602+711, 302+602+754, 302+602+760, 302+602+781, 302+602+786, 302+602+797, 302+602+834, 302+602+835, 302+605+609, 302+605+676, 202+605+694, 302+605+698, 302+605+699, 302+605+711, 302+605+754, 302+605+760, 302+605+781, 302+605+786, 302+605+797, 302+605+834, 302+605+835, 302+609+676, 302+609+694, 302+609+698, 302+609+699, 302+609+711, 302+609+754, 302+609+760, 302+609+781, 302+609+786, 302+609+797, 302+609+834, 302+609+835, 302+676+694, 302+676+698, 302+676+699, 302+676+711, 302+676+754, 302+676+760, 302+676+781, 302+676+786, 302+676+797, 302+676+834, 302+676+835, 302+694+698, 302+694+699, 302+694+711, 302+694+754, 302+694+760, 302+694+781, 302+694+786, 302+694+797, 302+694+834, 302+694+835, 302+698+699, 302+698+711, 302+698+754, 302+698+760, 302+698+781, 302+698+786, 302+698+797, 302+698+834, 302+698+835, 302+699+711, 302+699+754, 302+699+760, 302+699+781, 302+699+786, 302+699+797, 302+699+834, 302+699+835, 302+711+754, 302+711+760, 302+711+781, 302+711+786, 302+711+797, 302+711+834, 302+711+835, 302+754+760, 302+754+781, 302+754+786, 302+754+797, 302+754+834, 302+754+835, 302+760+781, 302+760+786, 302+760+797, 302+760+834, 302+760+835, 302+781+786, 302+781+797, 302+781+834, 302+781+835, 302+786+797, 302+786+834, 302+786+835, 302+797+834, 302+797+835, 302+834+835, 387+388+390, 387+388+403, 387+388+408, 387+388+410, 387+388+416, 387+388+448, 387+388+451, 387+388+471, 387+388+472, 387+388+507, 387+388+512, 387+388+515, 387+388+538, 387+388+598, 387+388+602, 387+388+605, 387+388+609, 387+388+676, 387+388+694, 387+388+698, 387+388+699, 387+388+711, 387+388+754, 387+388+760, 387+388+781, 387+388+786, 387+388+797, 387+388+834, 387+388+835, 387+390+403, 387+390+408, 387+390+410, 387+390+416, 387+390+448, 387+390+451, 387+390+471, 387+390+472, 387+390+507, 387+390+512, 387+390+515, 387+390+538, 387+390+598, 387+390+602, 387+390+605, 387+390+609, 387+390+676, 387+390+694, 387+390+698, 387+390+699, 387+390+711, 387+390+754, 87+390+760, 387+390+781, 387+390+786, 387+390+797, 387+390+834, 387+390+835, 387+403+408, 387+403+410, 387+403+416, 387+403+448, 387+403+451, 387+403+471, 387+403+472, 387+403+507, 387+403+512, 387+403+515, 387+403+538, 387+403+598, 387+403+602, 387+403+605, 387+403+609, 387+403+676, 387+403+694, 387+403+698, 387+403+699, 387+403+711, 387+403+754, 387+403+760, 387+403+781, 387+403+786, 387+403+797, 387+403+834, 387+403+835, 387+408+410, 387+408+416, 387+408+448, 387+408+451, 387+408+471, 387+408+472, 387+408+507, 387+408+512, 387+408+515, 387+408+538, 387+408+598, 387+408+602, 387+408+605, 387+408+609, 387+408+676, 387+408+694, 387+408+698, 387+408+699, 387+408+711, 387+408+754, 387+408+760, 387+408+781, 387+408+786, 387+408+797, 387+408+834, 387+408+835, 387+410+416, 387+410+448, 387+410+451, 387+410+471, 87+410+472, 387+410+507, 387+410+512, 387+410+515, 387+410+538, 387+410+598, 387+410+602, 387+410+605, 387+410+609, 387+410+676, 387+410+694, 387+410+698, 387+410+699, 387+410+711, 387+410+754, 387+410+760, 387+410+781, 387+410+786, 387+410+797, 387+410+834, 387+410+835, 387+416+448, 387+416+451, 387+416+471, 387+416+472, 387+416+507, 387+416+512, 387+416+515, 387+416+538, 387+416+598, 387+416+602, 387+416+605, 387+416+609, 387+416+676, 387+416+694, 387+416+698, 387+416+699, 387+416+711, 387+416+754, 387+416+760, 387+416+781, 387+416+786, 387+416+797, 387+416+834, 387+416+835, 387+448+451, 387+448+471, 387+448+472, 387+448+507, 387+448+512, 387+448+515, 387+448+538, 387+448+598, 387+448+602, 387+448+605, 387+448+609, 387+448+676, 387+448+694, 387+448+698, 387+448+699, 387+448+711, 387+448+754, 387+448+760, 387+448+781, 387+448+786, 387+448+797, 387+448+834, 387+448+835, 387+451+471, 387+451+472, 387+451+507, 387+451+512, 387+451+515, 387+451+538, 387+451+598, 387+451+602, 387+451+605, 387+451+609, 387+451+676, 387+451+694, 387+451+698, 387+451+699, 387+451+711, 387+451+754, 387+451+760, 387+451+781, 387+451+786, 387+451+797, 387+451+834, 387+451+835, 387+471+472, 387+471+507, 387+471+512, 387+471+515, 387+471+538, 387+471+598, 387+471+602, 387+471+605, 387+471+609, 387+471+676, 387+471+694, 387+471+698, 387+471+699, 387+471+711, 387+471+754, 387+471+760, 387+471+781, 387+471+786, 387+471+797, 387+471+834, 387+471+835, 387+472+507, 387+472+512, 387+472+515, 87+472+538, 387+472+598, 387+472+602, 387+472+605, 387+472+609, 387+472+676, 387+472+694, 387+472+698, 387+472+699, 387+472+711, 387+472+754, 387+472+760, 387+472+781, 387+472+786, 387+472+797, 387+472+834, 387+472+835, 387+507+512, 387+507+515, 387+507+538, 387+507+598, 387+507+602, 387+507+605, 387+507+609, 387+507+676, 387+507+694, 387+507+698, 387+507+699, 387+507+711, 387+507+754, 387+507+760, 387+507+781, 387+507+786, 387+507+797, 387+507+834, 387+507+835, 387+512+515, 387+512+538, 387+512+598, 387+512+602, 387+512+605, 387+512+609, 387+512+676, 387+512+694, 387+512+698, 387+512+699, 387+512+711, 387+512+754, 387+512+760, 387+512+781, 387+512+786, 387+512+797, 387+512+834, 387+512+835, 387+515+538, 387+515+598, 387+515+602, 387+515+605, 387+515+609, 387+515+676, 387+515+694, 387+515+698, 387+515+699, 387+515+711, 387+515+754, 387+515+760, 387+515+781, 387+515+786, 387+515+797, 387+515+834, 387+515+835, 387+538+598, 387+538+602, 387+538+605, 387+538+609, 387+538+676, 387+538+694, 387+538+698, 387+538+699, 387+538+711, 387+538+754, 387+538+760, 387+538+781, 387+538+786, 387+538+797, 387+538+834, 387+538+835, 387+598+602, 387+598+605, 387+598+609, 387+598+676, 387+598+694, 387+598+698, 387+598+699, 387+598+711, 387+598+754, 387+598+760, 387+598+781, 387+598+786, 387+598+797, 387+598+834, 387+598+835, 387+602+605, 387+602+609, 387+602+676, 387+602+694, 387+602+698, 387+602+699, 387+602+711, 387+602+754, 387+602+760, 387+602+781, 387+602+786, 387+602+797, 387+602+834, 387+602+835, 387+605+609, 387+605+676, 387+605+694, 387+605+698, 387+605+699, 387+605+711, 387+605+754, 387+605+760, 387+605+781, 387+605+786, 387+605+797, 387+605+834, 387+605+835, 387+609+676, 387+609+694, 387+609+698, 387+609+699, 387+609+711, 387+609+754, 387+609+760, 387+609+781, 387+609+786, 387+609+797, 387+609+834, 387+609+835, 387+676+694, 387+676+698, 387+676+699, 387+676+711, 387+676+754, 387+676+760, 387+676+781, 387+676+786, 387+676+797, 387+676+834, 387+676+835, 387+694+698, 387+694+699, 387+694+711, 387+694+754, 387+694+760, 387+694+781, 387+694+786, 387+694+797, 387+694+834, 387+694+835, 387+698+699, 387+698+711, 387+698+754, 387+698+760, 387+698+781, 387+698+786, 387+698+797, 387+698+834, 387+698+835, 387+699+711, 387+699+754, 387+699+760, 387+699+781, 387+699+786, 387+699+797, 387+699+834, 387+699+835, 387+711+754, 387+711+760, 387+711+781, 387+711+786, 387+711+797, 387+711+834, 387+711+835, 387+754+760, 387+754+781, 387+754+786, 387+754+797, 387+754+834, 387+754+835, 387+760+781, 387+760+786, 387+760+797, 387+760+834, 387+760+835, 387+781+786, 387+781+797, 387+781+834, 387+781+835, 387+786+797, 387+786+834, 387+786+835, 387+797+834, 387+797+835, 387+834+835, 388+390+403, 388+390+408, 388+390+410, 388+390+416, 388+390+448, 388+390+451, 388+390+471, 388+390+472, 388+390+507, 388+390+512, 388+390+515, 388+390+538, 388+390+598, 388+390+602, 388+390+605, 388+390+609, 388+390+676, 388+390+694, 388+390+698, 388+390+699, 388+390+711, 388+390+754, 388+390+760, 388+390+781, 388+390+786, 388+390+797, 388+390+834, 388+390+835, 388+403+408, 388+403+410, 388+403+416, 388+403+448, 388+403+451, 388+403+471, 388+403+472, 388+403+507, 388+403+512, 388+403+515, 388+403+538, 388+403+598, 388+403+602, 388+403+605, 388+403+609, 388+403+676, 388+403+694, 388+403+698, 388+403+699, 388+403+711, 388+403+754, 388+403+760, 388+403+781, 388+403+786, 388+403+797, 388+403+834, 388+403+835, 388+408+410, 388+408+416, 388+408+448, 388+408+451, 388+408+471, 388+408+472, 388+408+507, 388+408+512, 388+408+515, 388+408+538, 388+408+598, 388+408+602, 388+408+605, 388+408+609, 388+408+676, 388+408+694, 388+408+698, 388+408+699, 388+408+711, 388+408+754, 388+408+760, 388+408+781, 388+408+786, 388+408+797, 388+408+834, 388+408+835, 388+410+416, 388+410+448, 388+410+471, 388+410+471, 388+410+472, 388+410+507, 388+410+512, 388+410+515, 388+410+538, 388+410+598, 388+410+602, 388+410+605, 388+410+609, 388+410+676, 388+410+694, 388+410+698, 388+410+699, 388+410+711, 388+410+754, 388+410+760, 388+410+781, 388+410+786, 388+410+797, 388+410+834, 388+410+835, 388+416+448, 388+416+451, 388+416+471, 388+416+472, 388+416+507, 388+416+512, 388+416+515, 388+416+538, 388+416+598, 388+416+602, 388+416+605, 388+416+609, 388+416+676, 388+416+694, 388+416+698, 388+416+699, 388+416+711, 388+416+754, 388+416+760, 388+416+781, 388+416+786, 388+416+797, 388+416+834, 388+416+835, 388+448+451, 388+448+471, 388+448+472, 388+448+507, 388+448+512, 388+448+515, 388+448+538, 388+448+598, 388+448+602, 388+448+605, 388+448+609, 388+448+676, 388+448+694, 388+448+698, 388+448+699, 388+448+711, 388+448+754, 388+448+760, 388+448+781, 388+448+786, 388+448+797, 388+448+834, 388+448+835, 388+451+471, 388+451+472, 388+451+507, 388+451+512, 388+451+515, 388+451+538, 388+451+598, 388+451+602, 388+451+605, 388+451+609, 388+451+676, 388+451+694, 388+451+698, 388+451+699, 388+451+711, 388+451+754, 388+451+760, 388+451+781, 388+451+786, 388+451+797, 388+451+834, 388+451+835, 388+471+472, 388+471+507, 388+471+512, 388+471+515, 388+471+538, 388+471+598, 388+471+602, 388+471+605, 388+471+609, 388+471+676, 388+471+694, 388+471+698, 388+471+699, 388+471+711, 388+471+754, 388+471+760, 388+471+781, 388+471+786, 388+471+797, 388+471+834, 388+471+835, 388+472+507, 388+472+512, 388+472+515, 388+472+538, 388+472+598, 388+472+602, 388+472+605, 388+472+609, 388+472+676, 388+472+694, 388+472+698, 388+472+699, 388+472+711, 388+472+754, 388+472+760, 388+472+781, 388+472+786, 388+472+797, 388+472+834, 388+472+835, 388+507+512, 388+507+515, 388+507+538, 388+507+598, 388+507+602, 388+507+605, 388+507+609, 388+507+676, 388+507+694, 388+507+698, 388+507+699, 388+507+711, 388+507+754, 388+507+760, 388+507+781, 388+507+786, 388+507+797, 388+507+834, 388+507+835, 388+512+515, 388+512+538, 388+512+598, 388+512+602, 388+512+605, 388+512+609, 388+512+676, 388+512+694, 388+512+698, 388+512+699, 388+512+711, 388+512+754, 388+512+760, 388+512+781, 388+512+786, 388+512+797, 388+512+834, 388+512+835, 388+515+538, 388+515+598, 388+515+602, 388+515+605, 388+515+609, 388+515+676, 388+515+694, 388+515+698, 388+515+699, 388+515+711, 388+515+754, 388+515+760, 388+515+781, 388+515+786, 388+515+797, 388+515+834, 388+515+835, 388+538+598, 388+538+602, 388+538+605, 388+538+609, 388+538+676, 388+538+694, 388+538+698, 388+538+699, 388+538+711, 388+538+754, 388+538+760, 388+538+781, 388+538+786, 388+538+797, 388+538+834, 388+538+835, 388+598+602, 388+598+605, 388+598+609, 388+598+676, 388+598+694, 388+598+698, 388+598+699, 388+598+711, 388+598+754, 388+598+760, 388+598+781, 388+598+786, 388+598+797, 388+598+834, 388+598+835, 388+602+605, 388+602+609, 388+602+676, 388+602+694, 388+602+698, 388+602+699, 388+602+711, 388+602+754, 388+602+760, 388+602+781, 388+602+786, 388+602+797, 388+602+834, 388+602+835, 388+605+609, 388+605+676, 388+605+694, 388+605+698, 388+605+699, 388+605+711, 388+605+754, 388+605+760, 388+605+781, 388+605+786, 388+605+797, 388+605+834, 388+605+835, 388+609+676, 388+609+694, 388+609+698, 388+609+699, 388+609+711, 388+609+754, 388+609+760, 388+609+781, 388+609+786, 388+609+797, 388+609+834, 388+609+835, 388+676+694, 388+676+698, 388+676+699, 388+676+711, 388+676+754, 388+676+760, 388+676+781, 388+676+786, 388+676+797, 388+676+834, 388+676+835, 388+694+698, 388+694+699, 388+694+711, 388+694+754, 388+694+760, 388+694+781, 388+694+786, 388+694+797, 388+694+834, 388+694+835, 388+698+699, 388+698+711, 388+698+754, 388+698+760, 388+698+781, 388+698+786, 388+698+797, 388+698+834, 388+698+835, 388+699+711, 388+699+754, 388+699+760, 388+699+781, 388+699+786, 388+699+797, 388+699+834, 388+699+835, 388+711+754, 388+711+760, 388+711+781, 388+711+786, 388+711+797, 388+711+834, 388+711+835, 388+754+760, 388+754+781, 388+754+786, 388+754+797, 388+754+834, 388+754+835, 388+760+781, 388+760+786, 388+760+797, 388+760+834, 388+760+835, 388+781+786, 388+781+797, 388+781+834, 388+781+835, 388+786+797, 388+786+834, 388+786+835, 388+797+834, 388+797+835, 388+834+835, 390+403+408, 390+403+410, 390+403+416, 390+403+448, 390+403+451, 390+403+471, 390+403+472, 390+403+507, 390+403+512, 390+403+515, 390+403+538, 390+403+598, 390+403+602, 390+403+605, 390+403+609, 390+403+676, 390+403+694, 390+403+698, 390+403+699, 390+403+711, 390+403+754, 390+403+760, 390+403+781, 390+403+786, 390+403+797, 390+403+834, 390+403+835, 390+408+410, 390+408+416, 390+408+448, 390+408+451, 390+408+471, 390+408+472, 390+408+507, 390+408+512, 390+408+515, 390+408+538, 390+408+598, 390+408+602, 390+408+605, 390+408+609, 390+408+676, 390+408+694, 390+408+698, 390+408+699, 390+408+711, 390+408+754, 390+408+760, 390+408+781, 390+408+786, 390+408+797, 390+408+834, 390+408+835, 390+410+416, 390+410+448, 390+410+451, 390+410+471, 390+410+472, 390+410+507, 390+410+512, 390+410+515, 390+410+538, 390+410+598, 390+410+602, 390+410+605, 390+410+609, 390+410+676, 390+410+694, 390+410+698, 390+410+699, 390+410+711, 390+410+754, 390+410+760, 390+410+781, 390+410+786, 390+410+797, 390+410+834, 390+410+835, 390+416+448, 390+416+451, 390+416+471, 390+416+472, 390+416+507, 390+416+512, 390+416+515, 390+416+538, 390+416+598, 390+416+602, 390+416+605, 390+416+609, 390+416+676, 390+416+694, 390+416+698, 390+416+699, 390+416+711, 390+416+754, 390+416+760, 390+416+781, 390+416+786, 390+416+797, 390+416+834, 390+416+835, 390+448+451, 390+448+471, 390+448+472, 390+448+507, 390+448+512, 390+448+515, 390+448+538, 390+448+598, 390+448+602, 390+448+605, 390+448+609, 390+448+676, 390+448+694, 390+448+698, 390+448+699, 390+448+711, 390+448+754, 390+448+760, 390+448+781, 390+448+786, 390+448+797, 390+448+834, 390+448+835, 390+451+471, 390+451+472, 390+451+507, 390+451+512, 390+451+515, 390+451+538, 390+451+598, 390+451+602, 390+451+605, 390+451+609, 390+451+676, 390+451+694, 390+451+698, 390+451+699, 390+451+711, 390+451+754, 390+451+760, 390+451+781, 390+451+786, 390+451+797, 390+451+834, 390+451+835, 390+471+472, 390+471+507, 390+471+512, 390+471+515, 390+471+538, 390+471+598, 390+471+602, 390+471+605, 390+471+609, 390+471+676, 390+471+694, 390+471+698, 390+471+699, 390+471+711, 390+471+754, 390+471+760, 390+471+781, 390+471+786, 390+471+797, 390+471+834, 390+471+835, 390+472+507, 390+472+512, 390+472+515, 390+472+538, 390+472+598, 390+472+602, 390+472+605, 390+472+609, 390+472+676, 390+472+694, 390+472+698, 390+472+699, 390+472+711, 390+472+754, 390+472+760, 390+472+781, 390+472+786, 390+472+797, 390+472+834, 390+472+835, 390+507+512, 390+507+515, 390+507+538, 390+507+598, 390+507+602, 390+507+605, 390+507+609, 390+507+676, 390+507+694, 390+507+698, 390+507+699, 390+507+711, 390+507+754, 390+507+760, 390+507+781, 390+507+786, 390+507+797, 390+507+834, 390+507+835, 390+512+515, 390+512+538, 390+512+598, 390+512+602, 390+512+605, 390+512+609, 390+512+676, 390+512+694, 390+512+698, 390+512+699, 390+512+711, 390+512+754, 390+512+760, 390+512+781, 390+512+786, 390+512+797, 390+512+834, 390+512+835, 390+515+538, 390+515+598, 390+515+602, 390+515+605, 390+515+609, 390+515+676, 390+515+694, 390+515+698, 390+515+699, 390+515+711, 390+515+754, 90+515+760, 390+515+781, 390+515+786, 390+515+797, 390+515+834, 390+515+835, 390+538+598, 390+538+602, 390+538+605, 390+538+609, 390+538+676, 390+538+694, 390+538+698, 390+538+699, 390+538+711, 390+538+754, 390+538+760, 390+538+781, 390+538+786, 390+538+797, 390+538+834, 390+538+835, 390+598+602, 390+598+605, 390+598+609, 390+598+676, 390+598+694, 390+598+698, 390+598+699, 390+598+711, 390+598+754, 390+598+760, 390+598+781, 390+598+786, 390+598+797, 390+598+834, 390+598+835, 390+602+605, 390+602+609, 390+602+676, 390+602+694, 390+602+698, 390+602+699, 390+602+711, 390+602+754, 390+602+760, 390+602+781, 390+602+786, 390+602+797, 390+602+834, 390+602+835, 390+605+609, 390+605+676, 390+605+694, 390+605+698, 390+605+699, 390+605+711, 390+605+754, 390+605+760, 390+605+781, 390+605+786, 390+605+797, 390+605+834, 390+605+835, 390+609+676, 390+609+694, 390+609+698, 390+609+699, 390+609+711, 390+609+754, 390+609+760, 390+609+781, 390+609+786, 390+609+797, 390+609+834, 390+609+835, 390+676+694, 390+676+698, 390+676+699.390+676+711, 390+676+754, 390+676+760, 390+676+781, 390+676+786, 390+676+797, 390+676+834, 390+676+835, 390+694+698, 390+694+699, 390+694+711, 390+694+754, 390+694+760, 390+694+781, 390+694+786, 390+694+797, 390+694+834, 390+694+835, 390+698+699, 390+698+711, 390+698+754, 390+698+760, 390+698+781, 390+698+786, 390+698+797, 390+698+834, 390+698+835, 390+699+711, 390+699+754, 390+699+760, 390+699+781, 390+699+786, 390+699+797, 390+699+834, 390+699+835, 390+711+754, 390+711+760, 390+711+781, 390+711+786, 390+711+797, 390+711+834, 390+711+835, 390+754+760, 390+754+781, 390+754+786, 390+754+797, 390+754+834, 390+754+835, 390+760+781, 390+760+786, 390+760+797, 390+760+834, 390+760+835, 390+781+786, 390+781+797, 390+781+834, 390+781+835, 390+786+797, 390+786+834, 390+786+835, 390+797+834, 390+797+835, 390+834+835, 403+408+410, 403+408+416, 403+408+448, 403+408+451, 403+408+471, 403+408+472, 403+408+507, 403+408+512, 403+408+515, 403+408+538, 403+408+598, 403+408+602, 403+408+605, 403+408+609, 403+408+676, 403+408+694, 403+408+698, 403+408+699, 403+408+711, 403+408+754, 403+408+760, 403+408+781, 403+408+786, 403+408+797, 403+408+834, 403+408+835, 403+410+416, 403+410+448, 403+410+451, 403+410+471, 403+410+472, 403+410+507, 403+410+512, 403+410+515, 403+410+538, 403+410+598, 403+410+602, 403+410+605, 403+410+609, 403+410+676, 403+410+694, 403+410+698, 403+410+699, 403+410+711, 403+410+754, 403+410+760, 403+410+781, 403+410+786, 403+410+797, 403+410+834, 403+410+835, 403+416+448, 403+416+451, 403+416+471, 403+416+472, 403+416+507, 403+416+512, 403+416+515, 403+416+538, 403+416+598, 403+416+602, 403+416+605, 403+416+609, 403+416+676, 403+416+694, 403+416+698, 403+416+699, 403+416+711, 403+416+754, 403+416+760, 403+416+781, 403+416+786, 403+416+797, 403+416+834, 403+416+835, 403+448+451, 403+448+471, 403+448+472, 403+448+507, 403+448+512, 403+448+515, 403+448+538, 403+448+598, 403+448+602, 403+448+605, 403+448+609, 403+448+676, 403+448+694, 403+448+698, 403+448+699, 403+448+711, 403+448+754, 403+448+760, 403+448+781, 403+448+786, 403+448+797, 403+448+834, 403+448+835, 403+451+471, 403+451+472, 403+451+507, 403+451+512, 403+451+515.403+451+538, 403+451+598, 403+451+602, 403+451+605, 403+451+609, 403+451+676, 403+451+694, 403+451+698, 403+451+699, 403+451+711, 403+451+754, 403+451+760, 403+451+781, 403+451+786, 403+451+797, 403+451+834, 403+451+835, 403+471+472, 403+471+507, 403+471+512, 403+471+515, 403+471+538, 403+471+598, 403+471+602, 403+471+605, 403+471+609, 403+471+676, 403+471+694, 403+471+698, 403+471+699, 403+471+711, 403+471+754, 403+471+760, 403+471+781, 403+471+786, 403+471+797, 403+471+834, 403+471+835, 403+472+507, 403+472+512, 403+472+515, 403+472+538, 403+472+598, 403+472+602, 403+472+605, 403+472+609, 403+472+676, 403+472+694, 403+472+698, 403+472+699, 403+472+711, 403+472+754, 403+472+760, 403+472+781, 403+472+786, 403+472+797, 403+472+834, 403+472+835, 403+507+512, 403+507+515, 403+507+538, 403+507+598, 403+507+602, 403+507+605, 403+507+609, 403+507+676, 403+507+694, 403+507+698, 403+507+699, 403+507+711, 403+507+754, 403+507+760, 403+507+781, 403+507+786, 403+507+797, 403+507+834, 403+507+835, 403+512+515, 403+512+538, 403+512+598, 403+512+602, 403+512+605, 403+512+609, 403+512+676, 403+512+694, 403+512+698, 403+512+699, 403+512+711, 403+512+754, 403+512+760, 403+512+781, 403+512+786, 403+512+797, 403+512+834, 403+512+835, 403+515+538, 403+515+598, 403+515+602, 403+515+605, 403+515+609, 403+515+676, 403+515+694, 403+515+698, 403+515+699, 403+515+711, 403+515+754, 403+515+760, 403+515+781, 403+515+786, 403+515+797, 403+515+834, 403+515+835, 403+538+598, 403+538+602, 403+538+605, 403+538+609, 403+538+676, 403+538+694, 403+538+698, 403+538+699, 403+538+711, 403+538+754, 403+538+760, 403+538+781, 403+538+786, 403+538+797, 403+538+834, 403+538+835, 403+598+602, 403+598+605, 403+598+609, 403+598+676, 403+598+694, 403+598+698, 403+598+699, 403+598+711, 403+598+754, 403+598+760, 403+598+781, 403+598+786, 403+598+797, 403+598+834, 403+598+835, 403+602+605, 403+602+609, 403+602+676, 403+602+694, 403+602+698, 403+602+699, 403+602+711, 403+602+754, 403+602+760, 403+602+781, 403+602+786, 403+602+797, 403+602+834, 403+602+835, 403+605+609, 403+605+676, 403+605+694, 403+605+698, 403+605+699, 403+605+711, 403+605+754, 403+605+760, 403+605+781, 403+605+786, 403+605+797, 403+605+834, 403+605+835, 403+609+676, 403+609+694, 403+609+698, 403+609+699, 403+609+711, 403+609+754, 403+609+760, 403+609+781, 403+609+786, 403+609+797, 403+609+834, 403+609+835, 403+676+694, 403+676+698, 403+676+699, 403+676+711, 403+676+754, 403+676+760, 403+676+781, 403+676+786, 403+676+797, 403+676+834, 403+676+835, 403+694+698, 403+694+699, 403+694+711, 403+694+754, 403+694+760, 403+694+781, 403+694+786, 403+694+797, 403+694+834, 403+694+835, 403+698+699, 403+698+711, 403+698+754, 403+698+760, 403+698+781, 403+698+786, 403+698+797, 403+698+834, 403+698+835, 403+699+711, 403+699+754, 403+699+760, 403+699+781, 403+699+786, 403+699+797, 403+699+834, 403+699+835, 403+711+754, 403+711+760, 403+711+781, 403+711+786, 403+711+797, 403+711+834, 403+711+835, 403+754+760, 403+754+781, 403+754+786, 403+754+797, 403+754+834, 403+754+835, 403+760+781, 403+760+786, 403+760+797, 403+760+834, 403+760+835, 403+781+786, 403+781+797, 403+781+834, 403+781+835, 403+786+797, 403+786+834, 403+786+835, 403+797+834, 403+797+835, 403+834+835, 408+410+416, 408+410+448, 408+410+451, 408+410+471, 408+410+472, 408+410+507, 408+410+512, 408+410+515, 408+410+538, 408+410+598, 408+410+602, 408+410+605, 408+410+609, 408+410+676, 408+410+694, 408+410+698, 408+410+699, 408+410+711, 408+410+754, 408+410+760, 408+410+781, 408+410+786, 408+410+797, 408+410+834, 408+410+835, 408+416+448, 408+416+451, 408+416+471, 408+416+472, 408+416+507, 408+416+512, 408+416+515, 408+416+538, 408+416+598, 408+416+602, 408+416+605, 408+416+609, 408+416+676, 408+416+694, 408+416+698, 408+416+699, 408+416+711, 408+416+754, 408+416+760, 408+416+781, 408+416+786, 408+416+797, 408+416+834, 408+416+835, 408+448+451, 408+448+471, 408+448+472, 408+448+507, 408+448+512, 408+448+515, 408+448+538, 408+448+598, 408+448+602, 408+448+605, 408+448+609, 408+448+676, 408+448+694, 408+448+698, 408+448+699, 408+448+711, 408+448+754, 408+448+760, 408+448+781, 408+448+786, 408+448+797, 408+448+834, 408+448+835, 408+451+471, 408+451+472, 408+451+507, 408+451+512, 408+451+515, 408+451+538, 408+451+598, 408+451+602, 408+451+605, 408+451+609, 408+451+676, 408+451+694, 408+451+698, 408+451+699, 408+451+711, 408+451+754, 408+451+760, 408+451+781, 408+451+786, 408+451+797, 408+451+834, 408+451+835, 408+471+472, 408+471+507, 408+471+512, 408+471+515, 408+471+538, 408+471+598, 408+471+602, 408+471+605, 408+471+609, 408+471+676, 408+471+694, 408+471+698, 408+471+699, 408+471+711, 408+471+754, 408+471+760, 408+471+781, 408+471+786, 408+471+797, 408+471+834, 408+471+835, 408+472+507, 408+472+512, 408+472+515, 408+472+538, 408+472+598, 408+472+602, 408+472+605, 408+472+609, 408+472+676, 408+472+694, 408+472+698, 408+472+699, 408+472+711, 408+472+754, 408+472+760, 408+472+781, 408+472+786, 408+472+797, 408+472+834, 408+472+835, 408+507+512, 408+507+515, 408+507+538, 408+507+598, 408+507+602, 408+507+605, 408+507+609, 408+507+676, 408+507+694, 408+507+698, 408+507+699, 408+507+711, 408+507+754, 408+507+760, 408+507+781, 408+507+786, 408+507+797, 408+507+834, 408+507+835, 408+512+515, 408+512+538, 408+512+598, 408+512+602, 408+512+605, 408+512+609, 408+512+676, 408+512+694, 408+512+698, 408+512+699, 408+512+711, 408+512+754, 408+512+760, 408+512+781, 408+512+786, 408+512+797, 408+512+834, 408+512+835, 408+515+538, 408+515+598, 408+515+602, 408+515+605, 408+515+609, 408+515+676, 408+515+694, 408+515+698, 408+515+699, 408+515+711, 408+515+754, 408+515+760, 408+515+781, 408+515+786, 408+515+797, 408+515+834, 408+515+835, 408+538+598, 408+538+602, 408+538+605, 408+538+609, 408+538+676, 408+538+694, 408+538+698, 408+538+699, 408+538+711, 408+538+754, 408+538+760, 408+538+781, 408+538+786, 408+538+797, 408+538+834, 408+538+835, 408+598+602, 408+598+605, 408+598+609, 408+598+676, 408+598+694, 408+598+698, 408+598+699, 408+598+711, 408+598+754, 408+598+760, 408+598+781, 408+598+786, 408+598+797, 408+598+834, 408+598+835, 408+602+605, 408+602+609, 408+602+676, 408+602+694, 408+602+698, 408+602+699, 408+602+711, 408+602+754, 408+602+760, 408+602+781, 408+602+786, 408+602+797, 408+602+834, 408+602+835, 408+605+609, 408+605+676, 408+605+694, 408+605+698, 408+605+699, 408+605+711, 408+605+754, 408+605+760, 408+605+781, 408+605+786, 408+605+797, 408+605+834, 408+605+835, 408+609+676, 408+609+694, 408+609+698, 408+609+699, 408+609+711, 408+609+754, 408+609+760, 408+609+781, 408+609+786, 408+609+797, 408+609+834, 408+609+835, 408+676+694, 408+676+698, 408+676+699, 408+676+711, 408+676+754, 408+676+760, 408+676+781, 408+676+786, 408+676+797, 408+676+834, 408+676+835, 408+694+698, 408+694+699, 408+694+711, 408+694+754, 408+694+760, 408+694+781, 408+694+786, 408+694+797, 408+694+834, 408+694+835, 408+698+699, 408+698+711, 408+698+754, 408+698+760, 408+698+781, 408+698+786, 408+698+797, 408+698+834, 408+698+835, 408+699+711, 408+699+754, 408+699+760, 408+699+781, 408+699+786, 408+699+797, 408+699+834, 408+699+835, 408+711+754, 408+711+760, 408+711+781, 408+711+786, 408+711+797, 408+711+834, 408+711+835, 408+754+760, 408+754+781, 408+754+786, 408+754+797, 408+754+834, 408+754+835, 408+760+781, 408+760+786, 408+760+797, 408+760+834, 408+760+835, 408+781+786, 408+781+797, 408+781+834, 408+781+835, 408+786+797, 408+786+834, 408+786+835, 408+797+834, 408+797+835, 408+834+835, 410+416+448, 410+416+451, 410+416+471, 410+416+472, 410+416+507, 410+416+512, 410+416+515, 410+416+538, 410+416+598, 410+416+602, 410+416+605, 410+416+609, 410+416+676, 410+416+694, 410+416+698, 410+416+699, 410+416+711, 410+416+754, 410+416+760, 410+416+781, 410+416+786, 410+416+797, 410+416+834, 410+416+835, 410+448+451, 410+448+471, 410+448+472, 410+448+507, 410+448+512, 410+448+515, 410+448+538, 410+448+598, 410+448+602, 410+448+605, 410+448+609, 410+448+676, 410+448+694, 410+448+698, 410+448+699, 410+448+711, 410+448+754, 410+448+760, 410+448+781, 410+448+786, 410+448+797, 410+448+834, 410+448+835, 410+451+471, 410+451+472, 410+451+507, 410+451+512, 410+451+515, 410+451+538, 410+451+598, 410+451+602, 410+451+605, 410+451+609, 410+451+676, 410+451+694, 410+451+698, 410+451+699, 410+451+711, 410+451+754, 410+451+760, 410+451+781, 410+451+786, 410+451+797, 410+451+834, 410+451+835, 410+471+472, 410+471+507, 410+471+512, 410+471+515, 410+471+538, 410+471+598, 410+471+602, 410+471+605, 410+471+609, 410+471+676, 410+471+694, 410+471+698, 410+471+699, 410+471+711, 410+471+754, 410+471+760, 410+471+781, 410+471+786, 410+471+797, 410+471+834, 410+471+835, 410+472+507, 410+472+512, 410+472+515, 410+472+538, 410+472+598, 410+472+602, 410+472+605, 410+472+609, 410+472+676, 410+472+694, 410+472+698, 410+472+699, 410+472+711, 410+472+754.410+472+760, 410+472+781, 410+472+786, 410+472+797, 410+472+834, 410+472+835, 410+507+512, 410+507+515, 410+507+538, 410+507+598, 410+507+602, 410+507+605, 410+507+609, 410+507+676, 410+507+694, 410+507+698, 410+507+699, 410+507+711, 410+507+754, 410+507+760, 410+507+781, 410+507+786, 410+507+797, 410+507+834, 410+507+835, 410+512+515, 410+512+538, 410+512+598, 410+512+602, 410+512+605, 410+512+609, 410+512+676, 410+512+694, 410+512+698, 410+512+699, 410+512+711, 410+512+754, 410+512+760, 410+512+781, 410+512+786, 410+512+797, 410+512+834, 410+512+835, 410+515+538, 410+515+598, 410+515+602, 410+515+605, 410+515+609, 410+515+676, 410+515+694, 410+515+698, 410+515+699, 410+515+711, 410+515+754, 410+515+760, 410+515+781, 410+515+786, 410+515+797, 410+515+834, 410+515+835, 410+538+598, 410+538+602, 410+538+605, 410+538+609, 410+538+676, 410+538+694, 410+538+698, 410+538+699, 410+538+711, 410+538+754, 410+538+760, 410+538+781, 410+538+786, 410+538+797, 410+538+834, 410+538+835, 410+598+602, 410+598+605, 410+598+609, 410+598+676, 410+598+694, 410+598+698, 410+598+699, 410+598+711, 410+598+754, 410+598+760, 410+598+781, 410+598+786, 410+598+797, 410+598+834, 410+598+835, 410+602+605, 410+602+609, 410+602+676, 410+602+694, 410+602+698, 410+602+699, 410+602+711, 410+602+754, 410+602+760, 410+602+781, 410+602+786, 410+602+797, 410+602+834, 410+602+835, 410+605+609, 410+605+676, 410+605+694, 410+605+698, 410+605+699, 410+605+711, 410+605+754, 410+605+760, 410+605+781, 410+605+786, 410+605+797, 410+605+834, 410+605+835, 410+609+676, 410+609+694, 410+609+698, 410+609+699, 410+609+711, 410+609+754, 410+609+760, 410+609+781, 410+609+786, 410+609+797, 410+609+834, 410+609+835, 410+676+694, 410+676+698, 410+676+699, 410+676+711, 410+676+754, 410+676+760, 410+676+781, 410+676+786, 410+676+797, 410+676+834, 410+676+835, 410+694+698, 410+694+699, 410+694+711, 410+694+754, 410+694+760, 410+694+781, 410+694+786, 410+694+797, 410+694+834, 410+694+835, 410+698+699, 410+698+711, 410+698+754, 410+698+760, 410+698+781, 410+698+786, 410+698+797, 410+698+834, 410+698+835, 410+699+711, 410+699+754, 410+699+760, 410+699+781, 410+699+786, 410+699+797, 410+699+834, 410+699+835, 410+711+754, 410+711+760, 410+711+781, 410+711+786, 410+711+797, 410+711+834, 410+711+835, 410+754+760, 410+754+781, 410+754+786, 410+754+797, 410+754+834, 410+754+835, 410+760+781, 410+760+786, 410+760+797, 410+760+834, 410+760+835, 410+781+786, 410+781+797, 410+781+834, 410+781+835, 410+786+797, 410+786+834, 410+786+835, 410+797+834, 410+797+835, 410+834+835, 416+448+451, 416+448+471, 416+448+472, 416+448+507, 416+448+512, 416+448+515, 416+448+538, 416+448+598, 416+448+602, 416+448+605, 416+448+609, 416+448+676, 416+448+694, 416+448+698, 416+448+699, 416+448+711, 416+448+754, 416+448+760, 416+448+781, 416+448+786, 416+448+797, 416+448+834, 416+448+835, 416+451+471, 416+451+472, 416+451+507, 416+451+512, 416+451+515, 416+451+538, 416+451+598, 416+451+602, 416+451+605, 416+451+609, 416+451+676, 416+451+694, 416+451+698, 416+451+699, 416+451+711, 416+451+754, 416+451+760, 416+451+781, 416+451+786, 416+451+797, 416+451+834, 416+451+835, 416+471+472, 416+471+507, 416+471+512, 416+471+515, 416+471+538, 416+471+598, 416+471+602, 416+471+605, 416+471+609, 416+471+676, 416+471+694, 416+471+698, 416+471+699, 416+471+711, 416+471+754, 416+471+760, 416+471+781, 416+471+786, 416+471+797, 416+471+834, 416+471+835, 416+472+507, 416+472+512, 416+472+515, 416+472+538, 416+472+598, 416+472+602, 416+472+605, 416+472+609, 416+472+676, 416+472+694, 416+472+698, 416+472+699, 416+472+711, 416+472+754, 416+472+760, 416+472+781, 416+472+786, 416+472+797, 416+472+834, 416+472+835, 416+507+512, 416+507+515, 416+507+538, 416+507+598, 416+507+602, 416+507+605, 416+507+609, 416+507+676, 416+507+694, 416+507+698, 416+507+699, 416+507+711, 416+507+754, 416+507+760, 416+507+781, 416+507+786, 416+507+797, 416+507+834, 416+507+835, 416+512+515, 416+512+538, 416+512+598, 416+512+602, 416+512+605, 416+512+609, 416+512+676, 416+512+694, 416+512+698, 416+512+699, 416+512+711, 416+512+754, 416+512+760, 416+512+781, 416+512+786, 416+512+797, 416+512+834, 416+512+835, 416+515+538, 416+515+598, 416+515+602, 416+515+605, 416+515+609, 416+515+676, 416+515+694, 416+515+698, 416+515+699, 416+515+711, 416+515+754, 416+515+760, 416+515+781, 416+515+786, 416+515+797, 416+515+834, 416+515+835, 416+538+598, 416+538+602, 416+538+605, 416+538+609, 416+538+676, 416+538+694, 416+538+698, 416+538+699, 416+538+711, 416+538+754, 416+538+760, 416+538+781, 416+538+786, 416+538+797, 416+538+834, 416+538+835, 416+598+602, 416+598+605, 416+598+609, 416+598+676, 416+598+694, 416+598+698, 416+598+699, 416+598+711, 416+598+754, 416+598+760, 416+598+781, 416+598+786, 416+598+797, 416+598+834, 416+598+835, 416+602+605, 416+602+609, 416+602+676, 416+602+694, 416+602+698, 416+602+699, 416+602+711, 416+602+754, 416+602+760, 416+602+781, 416+602+786, 416+602+797, 416+602+834, 416+602+835, 416+605+609, 416+605+676, 416+605+694, 416+605+698, 416+605+699, 416+605+711, 416+605+754, 416+605+760, 416+605+781, 416+605+786, 416+605+797, 416+605+834, 416+605+835, 416+609+676, 416+609+694, 416+609+698, 416+609+699, 416+609+711, 416+609+754, 416+609+760, 416+609+781, 416+609+786, 416+609+797, 416+609+834, 416+609+835, 416+676+694, 416+676+698, 416+676+699, 416+676+711, 416+676+754, 416+676+760, 416+676+781, 416+676+786, 416+676+797, 416+676+834, 416+676+835, 416+694+698, 416+694+699, 416+694+711, 416+694+754, 416+694+760, 416+694+781, 416+694+786, 416+694+797, 416+694+834, 416+694+835, 416+698+699, 416+698+711, 416+698+754, 416+698+760, 416+698+781, 416+698+786, 416+698+797, 416+698+834, 416+698+835, 416+699+711, 416+699+754, 416+699+760, 416+699+781, 416+699+786, 416+699+797, 416+699+834, 416+699+835, 416+711+754, 416+711+760, 416+711+781, 416+711+786, 416+711+797, 416+711+834, 416+711+835, 416+754+760, 416+754+781, 416+754+786, 416+754+797, 416+754+834, 416+754+835, 416+760+781, 416+760+786, 416+760+797, 416+760+834, 416+760+835, 416+781+786, 416+781+797, 416+781+834, 416+781+835, 416+786+797, 416+786+834, 416+786+835, 416+797+834, 416+797+835, 416+834+835, 448+451+471, 448+451+472, 448+451+507, 448+451+512, 448+451+515, 448+451+538, 448+451+598, 448+451+602, 448+451+605, 448+451+609, 448+451+676, 448+451+694, 448+451+698 448+451+699, 448+451+711, 448+451+754, 448+451+760, 448+451+781, 448+451+786, 448+451+797, 448+451+834, 448+451+835, 448+471+472, 448+471+507, 448+471+512, 448+471+515, 448+471+538, 448+471+598, 448+471+602, 448+471+605, 448+471+609, 448+471+676, 448+471+694, 448+471+698, 448+471+699, 448+471+711, 448+471+754, 448+471+760, 448+471+781, 448+471+786, 448+471+797, 448+471+834, 448+471+835, 448+472+507, 448+472+512, 448+472+515, 448+472+538, 448+472+598, 448+472+602, 448+472+605, 448+472+609, 448+472+676, 448+472+694, 448+472+698, 448+472+699, 448+472+711, 448+472+754, 448+472+760, 448+472+781, 448+472+786, 448+472+797, 448+472+834, 448+472+835, 448+507+512, 448+507+515, 448+507+538, 448+507+598, 448+507+602, 448+507+605, 448+507+609, 448+507+676, 448+507+694, 448+507+698, 448+507+699, 448+507+711, 448+507+754, 448+507+760, 448+507+781, 448+507+786, 448+507+797, 448+507+834, 448+507+835, 448+512+515, 448+512+538, 448+512+598, 448+512+602, 448+512+605, 448+512+609, 448+512+676, 448+512+694, 448+512+698, 448+512+699, 448+512+711, 448+512+754, 448+512+760, 448+512+781, 448+512+786, 448+512+797, 448+512+834, 448+512+835, 448+515+538, 448+515+598, 448+515+602, 448+515+605, 448+515+609, 448+515+676, 448+515+694, 448+515+698, 448+515+699, 448+515+711, 448+515+754, 448+515+760, 448+515+781, 448+515+786, 448+515+797, 448+515+834, 448+515+835, 448+538+598, 448+538+602, 448+538+605, 448+538+609, 448+538+676, 448+538+694, 448+538+698, 448+538+699, 448+538+711, 448+538+754, 448+538+760, 448+538+781, 448+538+786, 448+538+797, 448+538+834, 448+538+835, 448+598+602, 448+598+605, 448+598+609, 448+598+676, 448+598+694, 448+598+698, 448+598+699, 448+598+711, 448+598+754, 448+598+760, 448+598+781, 448+598+786, 448+598+797, 448+598+834, 448+598+835, 448+602+605, 448+602+609, 448+602+676, 448+602+694, 448+602+698, 448+602+699, 448+602+711, 448+602+754, 448+602+760, 448+602+781, 448+602+786, 448+602+797, 448+602+834, 448+602+835, 448+605+609, 448+605+676, 448+605+694, 448+605+698, 448+605+699, 448+605+711, 448+605+754, 448+605+760, 448+605+781, 448+605+786, 448+605+797, 448+605+834, 448+605+835, 448+609+676, 448+609+694, 448+609+698, 448+609+699, 448+609+711, 448+609+754, 448+609+760, 448+609+781, 448+609+786, 448+609+797, 448+609+834, 448+609+835, 448+676+694, 448+676+698, 448+676+699, 448+676+711, 448+676+754, 448+676+760, 448+676+781, 448+676+786, 448+676+797, 448+676+834, 448+676+835, 448+694+698, 448+694+699, 448+694+711, 448+694+754, 448+694+760, 448+694+781, 448+694+786, 448+694+797, 448+694+834, 448+694+835, 448+698+699, 448+698+711, 448+698+754, 448+698+760, 448+698+781, 448+698+786, 448+698+797, 448+698+834, 448+698+835, 448+699+711, 448+699+754, 448+699+760, 448+699+781, 448+699+786, 448+699+797, 448+699+834, 448+699+835, 448+711+754, 448+711+760, 448+711+781, 448+711+786, 448+711+797, 448+711+834, 448+711+835, 448+754+760, 448+754+781, 448+754+786, 448+754+797, 448+754+834, 448+754+835, 448+760+781, 448+760+786, 448+760+797, 448+760+834, 448+760+835, 448+781+786, 448+781+797, 448+781+834, 448+781+835, 448+786+797, 448+786+834, 448+786+835, 448+797+834, 448+797+835, 448+834+835, 451+471+472, 451+471+507, 451+471+512, 451+471+515, 451+471+538, 451+471+598, 451+471+602, 451+471+605, 451+471+609, 451+471+676, 451+471+694, 451+471+698, 451+471+699, 451+471+711, 451+471+754, 451+471+760, 451+471+781, 451+471+786, 451+471+797, 451+471+834, 451+471+835, 451+472+507, 451+472+512, 451+472+515, 451+472+538, 451+472+598, 451+472+602, 451+472+605, 451+472+609, 451+472+676, 451+472+694, 451+472+698, 451+472+699, 451+472+711, 451+472+754, 451+472+760, 451+472+781, 451+472+786, 451+472+797, 451+472+834, 451+472+835, 451+507+512, 451+507+515, 451+507+538, 451+507+598, 451+507+602, 451+507+605, 451+507+609, 451+507+676, 451+507+694, 451+507+698, 451+507+699, 451+507+711, 451+507+754, 451+507+760, 451+507+781, 451+507+786, 451+507+797, 451+507+834, 451+507+835, 451+512+515, 451+512+538, 451+512+598, 451+512+602, 451+512+605, 451+512+609, 451+512+676, 451+512+694, 451+512+698, 451+512+699, 451+512+711, 451+512+754, 451+512+760, 451+512+781, 451+512+786, 451+512+797, 451+512+834, 451+512+835, 451+515+538, 451+515+598, 451+515+602, 451+515+605, 451+515+609, 451+515+676, 451+515+694, 451+515+698, 451+515+699, 451+515+711, 451+515+754, 451+515+760, 451+515+781, 451+515+786, 451+515+797, 451+515+834, 451+515+835, 451+538+598, 451+538+602, 451+538+605, 451+538+609, 451+538+676, 451+538+694, 451+538+698, 451+538+699, 451+538+711, 451+538+754, 451+538+760, 451+538+781, 451+538+786, 451+538+797, 451+538+834, 451+538+835, 451+598+602, 451+598+605, 451+598+609, 451+598+676, 451+598+694, 451+598+698, 451+598+699, 451+598+711, 451+598+754, 451+598+760, 451+598+781, 451+598+786, 451+598+797, 451+598+834, 451+598+835, 451+602+605, 451+602+609, 451+602+676, 451+602+694, 451+602+698, 451+602+699, 451+602+711, 451+602+754, 451+602+760, 451+602+781, 451+602+786, 451+602+797, 451+602+834, 451+602+835, 451+605+609, 451+605+676, 451+605+694, 451+605+698, 451+605+699, 451+605+711, 451+605+754, 451+605+760, 451+605+781, 451+605+786, 451+605+797, 451+605+834, 451+605+835, 451+609+676, 451+609+694, 451+609+698, 451+609+699, 451+609+711, 451+609+754, 451+609+760, 451+609+781, 451+609+786, 451+609+797, 451+609+834, 451+609+835, 451+676+694, 451+676+698, 451+676+699, 451+676+711, 451+676+754, 451+676+760, 451+676+781, 451+676+786, 451+676+797, 451+676+834, 451+676+835, 451+694+698, 451+694+699, 451+694+711, 451+694+754, 451+694+760, 451+694+781, 451+694+786, 451+694+797, 451+694+834, 451+694+835, 451+698+699, 451+698+711, 451+698+754, 451+698+760, 451+698+781, 451+698+786, 451+698+797, 451+698+834, 451+698+835, 451+699+711, 451+699+754, 451+699+760, 451+699+781, 451+699+786, 451+699+797, 451+699+834, 451+699+835, 451+711+754, 451+711+760, 451+711+781, 451+711+786, 451+711+797, 451+711+834, 451+711+835, 451+754+760, 451+754+781, 451+754+786, 451+754+797, 451+754+834, 451+754+835, 451+760+781, 451+760+786, 451+760+797, 451+760+834, 451+760+835, 451+781+786, 451+781+797, 451+781+834, 451+781+835, 451+786+797, 451+786+834, 451+786+835, 451+797+834, 451+797+835, 451+834+835, 471+472+507, 471+472+512, 471+472+515, 471+472+538, 471+472+598, 471+472+602, 471+472+605, 471+472+609, 471+472+676, 471+472+694, 471+472+698, 471+472+699, 471+472+711, 471+472+754, 471+472+760, 471+472+781, 471+472+786, 471+472+797, 471+472+834, 471+472+835, 471+507+512, 471+507+515, 471+507+538, 471+507+598, 471+507+602, 471+507+605, 471+507+609, 471+507+676, 471+507+694, 471+507+698, 471+507+699, 471+507+711, 471+507+754, 471+507+760, 471+507+781, 471+507+786, 471+507+797, 471+507+834, 471+507+835, 471+512+515, 471+512+538, 471+512+598, 471+512+602, 471+512+605, 471+512+609, 471+512+676, 471+512+694, 471+512+698, 471+512+699, 471+512+711, 471+512+754, 471+512+760, 471+512+781, 471+512+786, 471+512+797, 471+512+834, 471+512+835, 471+515+538, 471+515+598, 471+515+602, 471+515+605, 471+515+609, 471+515+676, 471+515+694, 471+515+698, 471+515+699, 471+515+711, 471+515+754, 471+515+760, 471+515+781, 471+515+786, 471+515+797, 471+515+834, 471+515+835, 471+538+598, 471+538+602, 471+538+605, 471+538+609, 471+538+676, 471+538+694, 471+538+698, 471+538+699, 471+538+711, 471+538+754, 471+538+760, 471+538+781, 471+538+786, 471+538+797, 471+538+834, 471+538+835, 471+598+602, 471+598+605, 471+598+609, 471+598+676, 471+598+694, 471+598+698, 471+598+699, 471+598+711, 471+598+754, 471+598+760, 471+598+781, 471+598+786, 471+598+797, 471+598+834, 471+598+835, 471+602+605, 471+602+609, 471+602+676, 471+602+694, 471+602+698, 471+602+699, 471+602+711, 471+602+754, 471+602+760, 471+602+781, 471+602+786, 471+602+797, 471+602+834, 471+602+835, 471+605+609, 471+605+676, 471+605+694, 471+605+698, 471+605+699, 471+605+711, 471+605+754, 471+605+760, 471+605+781, 471+605+786, 471+605+797, 471+605+834, 471+605+835, 471+609+676, 471+609+694, 471+609+698, 471+609+699, 471+609+711, 471+609+754, 471+609+760, 471+609+781, 471+609+786, 471+609+797, 471+609+834, 471+609+835, 471+676+694, 471+676+698, 471+676+699, 471+676+711, 471+676+754, 471+676+760, 471+676+781, 471+676+786, 471+676+797, 471+676+834, 471+676+835, 471+694+698, 471+694+699, 471+694+711, 471+694+754, 471+694+760, 471+694+781, 471+694+786, 471+694+797, 471+694+834, 471+694+835, 471+698+699, 471+698+711, 471+698+754, 471+698+760, 471+698+781, 471+698+786, 471+698+797, 471+698+834, 471+698+835, 471+699+711, 471+699+754, 471+699+760, 471+699+781, 471+699+786, 471+699+797, 471+699+834, 471+699+835, 471+711+754, 471+711+760, 471+711+781, 471+711+786, 471+711+797, 471+711+834, 471+711+835, 471+754+760, 471+754+781, 471+754+786, 471+754+797, 471+754+834, 471+754+835, 471+760+781, 471+760+786, 471+760+797, 471+760+834, 471+760+835, 471+781+786, 471+781+797, 471+781+834, 471+781+835, 471+786+797, 471+786+834, 471+786+835, 471+797+834, 471+797+835, 471+834+835, 472+507+512, 472+507+515, 472+507+538, 472+507+598, 472+507+602, 472+507+605, 472+507+609, 472+507+676, 472+507+694, 472+507+698, 472+507+699, 472+507+711, 472+507+754, 472+507+760, 472+507+781, 472+507+786, 472+507+797, 472+507+834, 472+507+835, 472+512+515, 472+512+538, 472+512+598, 472+512+602, 472+512+605, 472+512+609, 472+512+676, 472+512+694, 472+512+698, 472+512+699, 472+512+711, 472+512+754, 472+512+760, 472+512+781, 472+512+786, 472+512+797, 472+512+834, 472+512+835, 472+515+538, 472+515+598, 472+515+602, 472+515+605, 472+515+609, 472+515+676, 472+515+694, 472+515+698, 472+515+699, 472+515+711, 472+515+754, 472+515+760, 472+515+781, 472+515+786, 472+515+797, 472+515+834, 472+515+835, 472+538+598, 472+538+602, 472+538+605, 472+538+609, 472+538+676, 472+538+694, 472+538+698, 472+538+699, 472+538+711, 472+538+754, 472+538+760, 472+538+781, 472+538+786, 472+538+797, 472+538+834, 472+538+835, 472+598+602, 472+598+605, 472+598+609, 472+598+676, 472+598+694, 472+598+698, 472+598+699, 472+598+711, 472+598+754, 472+598+760, 472+598+781, 472+598+786, 472+598+797, 472+598+834, 472+598+835, 472+602+605, 472+602+609, 472+602+676, 472+602+694, 472+602+698, 472+602+699, 472+602+711, 472+602+754, 472+602+760, 472+602+781, 472+602+786, 472+602+797, 472+602+834, 472+602+835, 472+605+609, 472+605+676, 472+605+694, 472+605+698, 472+605+699, 472+605+711, 472+605+754, 472+605+760, 472+605+781, 472+605+786, 472+605+797, 472+605+834, 472+605+835, 472+609+676, 472+609+694, 472+609+698, 472+609+699, 472+609+711, 472+609+754, 472+609+760, 472+609+781, 472+609+786, 472+609+797, 472+609+834, 472+609+835, 472+676+694, 472+676+698, 472+676+699, 472+676+711, 472+676+754, 472+676+760, 472+676+781, 472+676+786, 472+676+797, 472+676+834, 472+676+835, 472+694+698, 472+694+699, 472+694+711, 472+694+754, 472+694+760, 472+694+781, 472+694+786, 472+694+797, 472+694+834, 472+694+835, 472+698+699, 472+698+711, 472+698+754, 472+698+760, 472+698+781, 472+698+786, 472+698+797, 472+698+834, 472+698+835, 472+699+711, 472+699+754, 472+699+760, 472+699+781, 472+699+786, 472+699+797, 472+699+834, 472+699+835, 472+711+754, 472+711+760, 472+711+781, 472+711+786, 472+711+797, 472+711+834, 472+711+835, 472+754+760, 472+754+781, 472+754+786, 472+754+797, 472+754+834, 472+754+835, 472+760+781, 472+760+786, 472+760+797, 472+760+834, 472+760+835, 472+781+786, 472+781+797, 472+781+834, 472+781+835, 472+786+797, 472+786+834, 472+786+835, 472+797+834, 472+797+835, 472+834+835, 507+512+515, 507+512+538, 507+512+598, 507+512+602, 507+512+605, 507+512+609, 507+512+676, 507+512+694, 507+512+698, 507+512+699, 507+512+711, 507+512+754, 507+512+760, 507+512+781, 507+512+786, 507+512+797, 507+512+834, 507+512+835, 507+515+538, 507+515+598, 507+515+602, 507+515+605, 507+515+609, 507+515+676, 507+515+694, 507+515+698, 507+515+699, 507+515+711, 507+515+754, 507+515+760, 507+515+781, 507+515+786, 507+515+797, 507+515+834, 507+515+835, 507+538+598, 507+538+602, 507+538+605, 507+538+609, 507+538+676, 507+538+694, 507+538+698, 507+538+699, 507+538+711, 507+538+754, 507+538+760, 507+538+781, 507+538+786, 507+538+797, 507+538+834, 507+538+835, 507+598+602, 507+598+605, 507+598+609, 507+598+676, 507+598+694, 507+598+698, 507+598+699, 507+598+711, 507+598+754, 507+598+760, 507+598+781, 507+598+786, 507+598+797, 507+598+834, 507+598+835, 507+602+605, 507+602+609, 507+602+676, 507+602+694, 507+602+698, 507+602+699, 507+602+711, 507+602+754, 507+602+760, 507+602+781, 507+602+786, 507+602+797, 507+602+834, 507+602+835, 507+605+609, 507+605+676, 507+605+694, 507+605+698, 507+605+699, 507+605+711, 507+605+754, 507+605+760, 507+605+781, 507+605+786, 507+605+797, 507+605+834, 507+605+835, 507+609+676, 507+609+694, 507+609+698, 507+609+699, 507+609+711, 507+609+754, 507+609+760, 507+609+781, 507+609+786, 507+609+797, 507+609+834, 507+609+835, 507+676+694, 507+676+698, 507+676+699, 507+676+711, 507+676+754, 507+676+760, 507+676+781, 507+676+786, 507+676+797, 507+676+834, 507+676+835, 507+694+698, 507+694+699, 507+694+711, 507+694+754, 507+694+760, 507+694+781, 507+694+786, 507+694+797, 507+694+834, 507+694+835, 507+698+699, 507+698+711, 507+698+754, 507+698+760, 507+698+781, 507+698+786, 507+698+797, 507+698+834, 507+698+835, 507+699+711, 507+699+754, 507+699+760, 507+699+781, 507+699+786, 507+699+797, 507+699+834, 507+699+835, 507+711+754, 507+711+760, 507+711+781, 507+711+786, 507+711+797, 507+711+834, 507+711+835, 507+754+760, 507+754+781, 507+754+786, 507+754+797, 507+754+834, 507+754+835, 507+760+781, 507+760+786, 507+760+797, 507+760+834, 507+760+835, 507+781+786, 507+781+797, 507+781+834, 507+781+835, 507+786+797, 507+786+834, 507+786+835, 507+797+834, 507+797+835, 507+834+835, 512+515+538, 512+515+598, 512+515+602, 512+515+605, 512+515+609, 512+515+676, 512+515+694, 512+515+698, 512+515+699, 512+515+711, 512+515+754, 512+515+760, 512+515+781, 512+515+786, 512+515+797, 512+515+834, 512+515+835, 512+538+598, 512+538+602, 512+538+605, 512+538+609, 512+538+676, 512+538+694, 512+538+698, 512+538+699, 512+538+711, 512+538+754, 512+538+760, 512+538+781, 512+538+786, 512+538+797, 512+538+834, 512+538+835, 512+598+602, 512+598+605, 512+598+609, 512+598+676, 512+598+694, 512+598+698, 512+598+699, 512+598+711, 512+598+754, 512+598+760, 512+598+781, 512+598+786, 512+598+797, 512+598+834, 512+598+835, 512+602+605, 512+602+609, 512+602+676, 512+602+694, 512+602+698, 512+602+699, 512+602+711, 512+602+754, 512+602+760, 512+602+781, 512+602+786, 512+602+797, 512+602+834, 512+602+835, 512+605+609, 512+605+676, 512+605+694, 512+605+698, 512+605+699, 512+605+711, 512+605+754, 512+605+760, 512+605+781, 512+605+786, 512+605+797, 512+605+834, 512+605+835, 512+609+676, 512+609+694, 512+609+698, 512+609+699, 512+609+711, 512+609+754, 512+609+760, 512+609+781, 512+609+786, 512+609+797, 512+609+834, 512+609+835, 512+676+694, 512+676+698, 512+676+699, 512+676+711, 512+676+754, 512+676+760, 512+676+781, 512+676+786, 512+676+797, 512+676+834, 512+676+835, 512+694+698, 512+694+699, 512+694+711, 512+694+754, 512+694+760, 512+694+781, 512+694+786, 512+694+797, 512+694+834, 512+694+835, 512+698+699, 512+698+711, 512+698+754, 512+698+760, 512+698+781, 512+698+786, 512+698+797, 512+698+834, 512+698+835, 512+699+711, 512+699+754, 512+699+760, 512+699+781, 512+699+786, 512+699+797, 512+699+834, 512+699+835, 512+711+754, 512+711+760, 512+711+781, 512+711+786, 512+711+797, 512+711+834, 512+711+835, 512+754+760, 512+754+781, 512+754+786, 512+754+797, 512+754+834, 512+754+835, 512+760+781, 512+760+786, 512+760+797, 512+760+834, 512+760+835, 512+781+786, 512+781+797, 512+781+834, 512+781+835, 512+786+797, 512+786+834, 512+786+835, 512+797+834, 512+797+835, 512+834+835, 515+538+598, 515+538+602, 515+538+605, 515+538+609, 515+538+676, 515+538+694, 515+538+698, 515+538+699, 515+538+711, 515+538+754, 515+538+760, 515+538+781, 515+538+786, 515+538+797, 515+538+834, 515+538+835, 515+598+602, 515+598+605, 515+598+609, 515+598+676, 515+598+694, 515+598+698, 515+598+699, 515+598+711, 515+598+754, 515+598+760, 515+598+781, 515+598+786, 515+598+797, 515+598+834, 515+598+835, 515+602+605, 515+602+609, 515+602+676, 515+602+694, 515+602+698, 515+602+699, 515+602+711, 515+602+754, 515+602+760, 515+602+781, 515+602+786, 515+602+797, 515+602+834, 515+602+835, 515+605+609, 515+605+676, 515+605+694, 515+605+698, 515+605+699, 515+605+711, 515+605+754, 515+605+760, 515+605+781, 515+605+786, 515+605+797, 515+605+834, 515+605+835, 515+609+676, 515+609+694, 515+609+698, 515+609+699, 515+609+711, 515+609+754, 515+609+760, 515+609+781, 515+609+786, 515+609+797, 515+609+834, 515+609+835, 515+676+694, 515+676+698, 515+676+699, 515+676+711, 515+676+754, 515+676+760, 515+676+781, 515+676+786, 515+676+797, 515+676+834, 515+676+835, 515+694+698, 515+694+699, 515+694+711, 515+694+754, 515+694+760, 515+694+781, 515+694+786, 515+694+797, 515+694+834, 515+694+835, 515+698+699, 515+698+711, 515+698+754, 515+698+760, 515+698+781, 515+698+786, 515+698+797, 515+698+834, 515+698+835, 515+699+711, 515+699+754, 515+699+760, 515+699+781, 515+699+786, 515+699+797, 515+699+834, 515+699+835, 515+711+754, 515+711+760, 515+711+781, 515+711+786, 515+711+797, 515+711+834, 515+711+835, 515+754+760, 515+754+781, 515+754+786, 515+754+797, 515+754+834, 515+754+835, 515+760+781, 515+760+786, 515+760+797, 515+760+834, 515+760+835, 515+781+786, 515+781+797, 515+781+834, 515+781+835, 515+786+797, 515+786+834, 515+786+835, 515+797+834, 515+797+835, 515+834+835, 538+598+602, 538+598+605, 538+598+609, 538+598+676, 538+598+694, 538+598+698, 538+598+699, 538+598+711, 538+598+754, 538+598+760, 538+598+781, 538+598+786, 538+598+797, 538+598+834, 538+598+835, 538+602+605, 538+602+609, 538+602+676, 538+602+694, 538+602+698, 538+602+699, 538+602+711, 538+602+754, 538+602+760, 538+602+781, 538+602+786, 538+602+797, 538+602+834, 538+602+835, 538+605+609, 538+605+676, 538+605+694, 538+605+698, 538+605+699, 538+605+711, 538+605+754, 538+605+760, 538+605+781, 538+605+786, 538+605+797, 538+605+834, 538+605+835, 538+609+676, 538+609+694, 538+609+698, 538+609+699, 538+609+711, 538+609+754, 538+609+760, 538+609+781, 538+609+786, 538+609+797, 538+609+834, 538+609+835, 538+676+694, 538+676+698, 538+676+699, 538+676+711, 538+676+754, 538+676+760, 538+676+781, 538+676+786, 538+676+797, 538+676+834, 538+676+835, 538+694+698, 538+694+699, 538+694+711, 538+694+754, 538+694+760, 538+694+781, 538+694+786, 538+694+797, 538+694+834, 538+694+835, 538+698+699, 538+698+711, 538+698+754, 538+698+760, 538+698+781, 538+698+786, 538+698+797, 538+698+834, 538+698+835, 538+699+711, 538+699+754, 538+699+760, 538+699+781, 538+699+786, 538+699+797, 538+699+834, 538+699+835, 538+711+754, 538+711+760, 538+711+781, 538+711+786, 538+711+797, 538+711+834, 538+711+835, 538+754+760, 538+754+781, 538+754+786, 538+754+797, 538+754+834, 538+754+835, 538+760+781, 538+760+786, 538+760+797, 538+760+834, 538+760+835, 538+781+786, 538+781+797, 538+781+834, 538+781+835, 538+786+797, 538+786+834, 538+786+835, 538+797+834, 538+797+835, 538+834+835, 58+602+605, 58+602+609, 58+602+676, 58+602+694, 598+602+698, 58+602+699, 58+602+711, 58+602+754, 58+602+760, 58+602+781, 598+602+786, 58+602+797, 58+602+834, 58+602+835, 58+605+609, 58+605+676, 598+605+694, 58+605+698, 58+605+699, 58+605+711, 58+605+754, 58+605+760, 598+605+781, 58+605+786, 58+605+797, 58+605+834, 58+605+835, 58+609+676, 598+609+694, 58+609+698, 58+609+699, 58+609+711, 58+609+754, 58+609+760, 598+609+781, 58+609+786, 58+609+797, 58+609+834, 58+609+835, 58+676+694, 598+676+698, 58+676+699, 58+676+711, 58+676+754, 58+676+760, 58+676+781, 598+676+786, 58+676+797, 58+676+834, 58+676+835, 58+694+698, 58+694+699, 598+694+711, 58+694+754, 58+694+760, 58+694+781, 58+694+786, 58+694+797, 598+694+834, 58+694+835, 58+698+699, 58+698+711, 58+698+754, 58+698+760, 598+698+781, 58+698+786, 58+698+797, 58+698+834, 58+698+835, 58+699+711, 598+699+754, 58+699+760, 58+699+781, 58+699+786, 58+699+797, 58+699+834, 598+699+835, 58+711+754, 58+711+760, 58+711+781, 58+711+786, 58+711+797, 598+711+834, 58+711+835, 58+754+760, 58+754+781, 58+754+786, 58+754+797, 598+754+834, 58+754+835, 58+760+781, 58+760+786, 58+760+797, 58+760+834, 598+760+835, 58+781+786, 58+781+797, 58+781+834, 58+781+835, 58+786+797, 598+786+834, 58+786+835, 58+797+834, 58+797+835, 58+834+835, 602+605+609, 602+605+676, 602+605+694, 602+605+698, 602+605+699, 602+605+711, 602+605+754, 602+605+760, 602+605+781, 602+605+786, 602+605+797, 602+605+834, 602+605+835, 602+609+676, 602+609+694, 602+609+698, 602+609+699, 602+609+711, 602+609+754, 602+609+760, 602+609+781, 602+609+786, 602+609+797, 602+609+834, 602+609+835, 602+676+694, 602+676+698, 602+676+699, 602+676+711, 602+676+754, 602+676+760, 602+676+781, 602+676+786, 602+676+797, 602+676+834, 602+676+835, 602+694+698, 602+694+699, 602+694+711, 602+694+754, 602+694+760, 602+694+781, 602+694+786, 602+694+797, 602+694+834, 602+694+835, 602+698+699, 602+698+711, 602+698+754, 602+698+760, 602+698+781, 602+698+786, 602+698+797, 602+698+834, 602+698+835, 602+699+711, 602+699+754, 602+699+760, 602+699+781, 602+699+786, 602+699+797, 602+699+834, 602+699+835, 602+711+754, 602+711+760, 602+711+781, 602+711+786, 602+711+797, 602+711+834, 602+711+835, 602+754+760, 602+754+781, 602+754+786, 602+754+797, 602+754+834, 602+754+835, 602+760+781, 602+760+786, 602+760+797, 602+760+834, 602+760+835, 602+781+786, 602+781+797, 602+781+834, 602+781+835, 602+786+797, 602+786+834, 602+786+835, 602+797+834, 602+797+835, 602+834+835, 605+609+676, 605+609+694, 605+609+698, 605+609+699, 605+609+711, 605+609+754, 605+609+760, 605+609+781, 605+609+786, 605+609+797, 605+609+834, 605+609+835, 605+676+694, 605+676+698, 605+676+699, 605+676+711, 605+676+754, 605+676+760, 605+676+781, 605+676+786, 605+676+797, 605+676+834, 605+676+835, 605+694+698, 605+694+699, 605+694+711, 605+694+754, 605+694+760, 605+694+781, 605+694+786, 605+694+797, 605+694+834, 605+694+835, 605+698+699, 605+698+711, 605+698+754, 605+698+760, 605+698+781, 605+698+786, 605+698+797, 605+698+834, 605+698+835, 605+699+711, 605+699+754, 605+699+760, 605+699+781, 605+699+786, 605+699+797, 605+699+834, 605+699+835, 605+711+754, 605+711+760, 605+711+781, 605+711+786, 605+711+797, 605+711+834, 605+711+835, 605+754+760, 605+754+781, 605+754+786, 605+754+797, 605+754+834, 605+754+835, 605+760+781, 605+760+786, 605+760+797, 605+760+834, 605+760+835, 605+781+786, 605+781+797, 605+781+834, 605+781+835, 605+786+797, 605+786+834, 605+786+835, 605+797+834, 605+797+835, 605+834+835, 609+676+694, 609+676+698, 609+676+699, 609+676+711, 609+676+754, 609+676+760, 609+676+781, 609+676+786, 609+676+797, 609+676+834, 609+676+835, 609+694+698, 609+694+699, 609+694+711, 609+694+754, 609+694+760, 609+694+781, 609+694+786, 609+694+797, 609+694+834, 609+694+835, 609+698+699, 609+698+711, 609+698+754, 609+698+760, 609+698+781, 609+698+786, 609+698+797, 609+698+834, 609+698+835, 609+699+711, 609+699+754, 609+699+760, 609+699+781, 609+699+786, 609+699+797, 609+699+834, 609+699+835, 609+711+754, 609+711+760, 609+711+781, 609+711+786, 609+711+797, 609+711+834, 609+711+835, 609+754+760, 609+754+781, 609+754+786, 609+754+797, 609+754+834, 609+754+835, 609+760+781, 609+760+786, 609+760+797, 609+760+834, 609+760+835, 609+781+786, 609+781+797, 609+781+834, 609+781+835, 609+786+797, 609+786+834, 609+786+835, 609+797+834, 609+797+835, 609+834+835, 676+694+698, 676+694+699, 676+694+711, 676+694+754, 676+694+760, 676+694+781, 676+694+786, 676+694+797, 676+694+834, 676+694+835, 676+698+699, 676+698+711, 676+698+754, 676+698+760, 676+698+781, 676+698+786, 676+698+797, 676+698+834, 676+698+835, 676+699+711, 676+699+754, 676+699+760, 676+699+781, 676+699+786, 676+699+797, 676+699+834, 676+699+835, 676+711+754, 676+711+760, 676+711+781, 676+711+786, 676+711+797, 676+711+834, 676+711+835, 676+754+760, 676+754+781, 676+754+786, 676+754+797, 676+754+834, 676+754+835, 676+760+781, 676+760+786, 676+760+797, 676+760+834, 676+760+835, 676+781+786, 676+781+797, 676+781+834, 676+781+835, 676+786+797, 676+786+834, 676+786+835, 676+797+834, 676+797+835, 676+834+835, 694+698+699, 694+698+711, 694+698+754, 694+698+760, 694+698+781, 694+698+786, 694+698+797, 694+698+834, 694+698+835, 694+699+711, 694+699+754, 694+699+760, 694+699+781, 694+699+786, 694+699+797, 694+699+834, 694+699+835, 694+711+754, 694+711+760, 694+711+781, 694+711+786, 694+711+797, 694+711+834, 694+711+835, 694+754+760, 694+754+781, 694+754+786, 694+754+797, 694+754+834, 694+754+835, 694+760+781, 694+760+786, 694+760+797, 694+760+834, 694+760+835, 694+781+786, 694+781+797, 694+781+834, 694+781+835, 694+786+797, 694+786+834, 694+786+835, 694+797+834, 694+797+835, 694+834+835, 698+699+711, 698+699+754, 698+699+760, 698+699+781, 698+699+786, 698+699+797, 698+699+834, 698+699+835, 968+711+754, 698+711+760, 698+711+781, 698+711+786, 698+711+797, 698+711+834, 698+711+835, 698+754+760, 698+754+781, 698+754+786, 698+754+797, 698+754+834, 698+754+835, 698+760+781, 698+760+786, 698+760+797, 698+760+834, 698+760+835, 698+781+786, 698+781+797, 698+781+834, 698+781+835, 698+786+797, 698+786+834, 698+786+835, 698+797+834, 698+797+835, 698+834+835, 699+711+754, 699+711+760, 699+711+781, 699+711+786, 699+711+797, 699+711+834, 699+711+835, 699+754+760, 699+754+781, 699+754+786, 699+754+797, 699+754+834, 699+754+835, 699+760+781, 699+760+786, 699+760+797, 699+760+834, 699+760+835, 699+781+786, 699+781+797, 699+781+834, 699+781+835, 699+786+797, 699+786+834, 699+786+835, 699+797+834, 699+797+835, 699+834+835, 711+754+760, 711+754+781, 711+754+786, 711+754+797, 711+754+834, 711+754+835, 711+760+781, 711+760+786, 711+760+797, 711+760+834, 711+760+835, 711+781+786, 711+781+797, 711+781+834, 711+781+835, 711+786+797, 711+786+834, 711+786+835, 711+797+834, 711+797+835, 711+834+835, 754+760+781, 754+760+786, 754+760+797, 754+760+834, 754+760+835, 754+781+786, 754+781+797, 754+781+834, 754+781+835, 754+786+797, 754+786+834, 754+786+835, 754+797+834, 754+797+835, 754+834+835, 760+781+786, 60+781+797, 760+781+834, 760+781+835, 760+786+797, 760+786+834, 760+786+835, 760+797+834, 760+797+835 760+834+835, 781+786+797,, 781+786+834,, 781+786+835,, 781+797+834,, 781+797+835,, 781+834+835, 786+797+834, 786+797+835, 786+834+835, and 797+834+835 of SEQ ID NO:2.

The concrete substitutions in the positions indicated in the above list are preferably those described above in relation to the individual positions.

Preferred are endoglucanase variants having an alteration (e.g. a substitution, deletion or insertion, preferably a substitution) in the positions selected from the group consisting of positions: 559+579+697; 512+559+579+697; 18+71+186+408+579+602+651+688+756; 18+189+408+559+579+688+697+756+921+934; 313+488; 880+905+921+934; 302+313+408+579+602+651+697+880+921+934; or 216+313+408+476+579+602+638+651+697+719+880+887+921+934 of SEQ ID NO:2.

Particularly preferred are endoglucanase variants having an alteration (e.g. a substitution, deletion or insertion, preferably a substitution) selected from the group consisting of the following alterations: A559N+Y579W+T697G; K512P+A559N+Y579W+T697G; N18G+A71E+A186P+E408D+Y579W+I602T+A651P+A688G+V756Y; N18G+N189K+E408D+A559N+Y579W+A688G+T697G+V756Y+K921R+Y934G; S313D+E408D; R880K+N905D+K921R+Y934G; I302D+S313D+E408D+Y579W+I602T+A651P+T697G+R880K+K921R+Y934G; and N216Q+S313D+E408D+D476R+Y579W+I602T+F638N+A651P+T697G+W719R+R880K+T887K+K9 21R+Y934G.

All of the above alterations are relative to SEQ ID NO:2.

In a particular embodiment, the invention relates to a detergent composition comprising an endoglucanase variant selected from the group consisting of the endoglucananase variants set forth in Tables 2-12 or 13 herein.

In a particular embodiment, the invention relates to a detergent composition comprising an endoglucananase variant selected from the group consisting of the endoglucananase variants set forth in Table 14 herein.

In a particular embodiment, the invention relates to a detergent composition comprising an endoglucananase variant selected from the group consisting of the endoglucananase variants set forth in Table 15 herein.

In a particular embodiment, the invention relates to a detergent composition comprising an endoglucananase variant selected from the group consisting of the endoglucananase variants set forth in Table 16 herein.

In one embodiment, the chelator-induced instability regions in the protein sequence of the known xanthan lyase having SEQ ID NO:6 that are affected when the molecule is incubated in a buffer with EDTA, are the following: region 1 corresponding to amino acids 154 to 176 of SEQ ID NO:6, region 2 corresponding to amino acids 614 to 658 of SEQ ID NO:6, region 3 corresponding to amino acids 731 to 803 of SEQ ID NO:6, region 4 corresponding to amino acids 807 to 846 of SEQ ID NO:6, region 5 corresponding to amino acids 872 to 885 of SEQ ID NO:6, and region 6 corresponding to amino acids 903 to 1004 of SEQ ID NO:6. This relates to an important guidance on where to mutate a xanthan lyase in order to stabilize the molecule in a detergent, e.g. detergent composition comprising a chelator, e.g. EDTA or citrate.

In one embodiment, the detergent composition comprises a xanthan lyase variant, comprising an alteration (e.g. a substitution, deletion or insertion, preferably a substitution) at one or more positions in a region selected from the group consisting of: region 1 corresponding to amino acids 154 to 176 of SEQ ID NO:6, region 2 corresponding to amino acids 614 to 658 of SEQ ID NO:6, region 3 corresponding to amino acids 731 to 803 of SEQ ID NO:6, region 4 corresponding to amino acids 807 to 846 of SEQ ID NO:6, region 5 corresponding to amino acids 872 to 885 of SEQ ID NO:6, and region 6 corresponding to amino acids 903 to 1004 of SEQ ID NO:6, wherein said variant has at least 60% and less than 100% sequence identity to SEQ ID NO:6; preferably said xanthan lyase variant has activity on xanthan gum, further preferably said activity is a xanthan gum degrading activity.

In one embodiment, the detergent composition comprises a xanthan lyase variant, comprising an alteration (e.g. a substitution, deletion or insertion, preferably a substitution) at one or more positions in two or more regions selected from the group consisting of: region 1 corresponding to amino acids 154 to 176 of SEQ ID NO:6, region 2 corresponding to amino acids 614 to 658 of SEQ ID NO:6, region 3 corresponding to amino acids 731 to 803 of SEQ ID NO:6, region 4 corresponding to amino acids 807 to 846 of SEQ ID NO:6, region 5 corresponding to amino acids 872 to 885 of SEQ ID NO:6, and region 6 corresponding to amino acids 903 to 1004 of SEQ ID NO:6, wherein said variant has at least 60% and less than 100% sequence identity to SEQ ID NO:6; preferably said xanthan lyase variant has activity on xanthan gum, further preferably said activity is a xanthan gum degrading activity.

In one embodiment, the detergent composition comprises a parent xanthan lyase as described herein (e.g. SEQ ID NO:6) having an alteration (e.g. a substitution, deletion or insertion, preferably a substitution) at one or more positions in a region selected from the group consisting of regions 1-6, wherein said region is a chelator-induced instability region, preferably said chelator-induced instability region has one or more of the following features: in the presence of a chelator it is less conformationally stable than one or more or all of its adjacent regions; and/or in the presence of a chelator it is more exposed to said chelator than one or more or all of its adjacent regions; and/or in the presence of a chelator it is more accessible to said chelator than one or more or all of its adjacent regions; and/or in the presence of a chelator it is more conformationally dynamic than one or more or all of its adjacent regions; and/or in the presence of a chelator it is more receptive to deuterium incorporation than one or more or all of its adjacent regions; further preferably said adjacent region is selected from the group consisting of: region 7 corresponding to amino acids 1 to 153 of SEQ ID NO:6, region 8 corresponding to amino acids 177 to 613 of SEQ ID NO:6, region 9 corresponding to amino acids 659 to 730 of SEQ ID NO:6, region 10 corresponding to amino acids 804 to 806 of SEQ ID NO:6, region 11 corresponding to amino acids 847 to 871 of SEQ ID NO:6, region 12 corresponding to amino acids 886 to 902 of SEQ ID NO:6, and region 13 corresponding to amino acids 1005 to 1037 of SEQ ID NO:6, further most preferably said chelator is EDTA or citrate.

In one embodiment the adjacent regions can be one or more or all of the following: region 7 corresponding to amino acids 1 to 153 of SEQ ID NO:6, region 8 corresponding to amino acids 177 to 613 of SEQ ID NO:6, region 9 corresponding to amino acids 659 to 730 of SEQ ID NO:6, region 10 corresponding to amino acids 804 to 806 of SEQ ID NO:6, region 11 corresponding to amino acids 847 to 871 of SEQ ID NO:6, region 12 corresponding to amino acids 886 to 902 of SEQ ID NO:6, and region 13 corresponding to amino acids 1005 to 1037 of SEQ ID NO:6.

In one embodiment, the detergent composition comprises a xanthan lyase variant having an alteration (e.g. a substitution, deletion or insertion, preferably a substitution) at one or more positions in a region selected from the group consisting of regions 1-6 (e.g. of SEQ ID NO:6 or another parent xanthan lyase), wherein in an aqueous solution comprising a detergent component said region (e.g. of SEQ ID NO:6 or another parent xanthan lyase) is relatively more accessible to said detergent component than one or more or all of its adjacent regions.

In one embodiment, the detergent composition comprises a xanthan lyase variant having an alteration (e.g. a substitution, deletion or insertion, preferably a substitution) at one or more positions in a region selected from the group consisting of regions 1-6 (e.g. of SEQ ID NO:6 or another parent xanthan lyase), wherein in an aqueous solution comprising a detergent component said region (e.g. of SEQ ID NO:6 or another parent xanthan lyase) is relatively more exposed to said detergent component than one or more or all of its adjacent regions.

In one embodiment, the detergent composition comprises a xanthan lyase variant having an alteration (e.g. a substitution, deletion or insertion, preferably a substitution) at one or more positions in a region selected from the group consisting of regions 1-6 (e.g. of SEQ ID NO:6 or another parent xanthan lyase), wherein in an aqueous solution comprising a detergent component said region (e.g. of SEQ ID NO:6 or another parent xanthan lyase) is relatively more conformationally dynamic than one or more or all of its adjacent regions.

In one embodiment, the detergent composition comprises a xanthan lyase variant having an alteration (e.g. a substitution, deletion or insertion, preferably a substitution) at one or more positions in a region selected from the group consisting of regions 1-6 (e.g. of SEQ ID NO:6 or another parent xanthan lyase), wherein in an aqueous solution comprising a detergent component said region (e.g. of SEQ ID NO:6 or another parent xanthan lyase) is relatively more receptive to deuterium incorporation than one or more or all of its adjacent regions.

In one embodiment, the detergent composition comprises a xanthan lyase variant of the invention, comprising an alteration (e.g. a substitution, deletion or insertion, preferably a substitution) at one or more positions in two or more regions selected from the group consisting of: region 1 corresponding to amino acids 154 to 176 of SEQ ID NO:6, region 2 corresponding to amino acids 614 to 658 of SEQ ID NO:6, region 3 corresponding to amino acids 731 to 803 of SEQ ID NO:6, region 4 corresponding to amino acids 807 to 846 of SEQ ID NO:6, region 5 corresponding to amino acids 872 to 885 of SEQ ID NO:6, and region 6 corresponding to amino acids 903 to 1004 of SEQ ID NO:6, wherein said variant has at least 60% and less than 100% sequence identity to SEQ ID NO:6, preferably said variant has activity on xanthan gum, further preferably said activity is a xanthan gum degrading activity.

In one embodiment, the detergent composition comprises a xanthan lyase variant having multiple alterations (such as 2, 3, 4, 5, 6, 7, 8, 9 or 10) in one region (e.g. of SEQ ID NO:6 or another parent xanthan lyase) selected from the group consisting of: region 1 corresponding to amino acids 154 to 176 of SEQ ID NO:6, region 2 corresponding to amino acids 614 to 658 of SEQ ID NO:6, region 3 corresponding to amino acids 731 to 803 of SEQ ID NO:6, region 4 corresponding to amino acids 807 to 846 of SEQ ID NO:6, region 5 corresponding to amino acids 872 to 885 of SEQ ID NO:6, and region 6 corresponding to amino acids 903 to 1004 of SEQ ID NO:6, wherein said variant has at least 60% and less than 100% sequence identity to SEQ ID NO:6, preferably said variant has activity on xanthan gum, further preferably said activity is a xanthan gum degrading activity.

In one embodiment, the detergent composition comprises a xanthan lyase variant having multiple alterations (e.g. 2, 3, 4, 5, 6, 7, 8, 9 or 10) in multiple regions (e.g. 2, 3, 4, 5, 6, 7, 8, 9 or 10) (e.g. of SEQ ID NO:6 or another parent xanthan lyase) selected from the group consisting of: region 1 corresponding to amino acids 154 to 176 of SEQ ID NO:6, region 2 corresponding to amino acids 614 to 658 of SEQ ID NO:6, region 3 corresponding to amino acids 731 to 803 of SEQ ID NO:6, region 4 corresponding to amino acids 807 to 846 of SEQ ID NO:6, region 5 corresponding to amino acids 872 to 885 of SEQ ID NO:6, and region 6 corresponding to amino acids 903 to 1004 of SEQ ID NO:6, wherein said variant has at least 60% and less than 100% sequence identity to SEQ ID NO:6, preferably said variant has activity on xanthan gum, further preferably said activity is a xanthan gum degrading activity.

In one embodiment, the regions in the protein sequence of the known xanthan lyase having SEQ ID NO:6 that have an impact on stability of the molecule, e.g. during storage in a liquid detergent composition, are the following: region 7 corresponding to amino acids 1 to 153 of SEQ ID NO:6, region 8 corresponding to amino acids 177 to 613 of SEQ ID NO:6, region 9 corresponding to amino acids 659 to 730 of SEQ ID NO:6, region 10 corresponding to amino acids 804 to 806 of SEQ ID NO:6, region 11 corresponding to amino acids 847 to 871 of SEQ ID NO:6, region 12 corresponding to amino acids 886 to 902 of SEQ ID NO:6, and region 13 corresponding to amino acids 1005 to 1037 of SEQ ID NO:6. This embodiment relates to an important guidance on where to mutate a xanthan lyase in order to stabilize the molecule in a detergent.

In one embodiment, the detergent compositions comprises a xanthan lyase variant, comprising an alteration (e.g. a substitution, deletion or insertion, preferably a substitution) at one or more positions in a region selected from the group consisting of: region 7 corresponding to amino acids 1 to 153 of SEQ ID NO:6, region 8 corresponding to amino acids 177 to 613 of SEQ ID NO:6, region 9 corresponding to amino acids 659 to 730 of SEQ ID NO:6, region 10 corresponding to amino acids 804 to 806 of SEQ ID NO:6, region 11 corresponding to amino acids 847 to 871 of SEQ ID NO:6, region 12 corresponding to amino acids 886 to 902 of SEQ ID NO:6, and region 13 corresponding to amino acids 1005 to 1037 of SEQ ID NO:6, wherein said variant has at least 60% and less than 100% sequence identity to SEQ ID NO:6; preferably said xanthan lyase variant has activity on xanthan gum, further preferably said activity is a xanthan gum degrading activity.

In one embodiment, the detergent compositions comprise a xanthan lyase variant, comprising an alteration (e.g. a substitution, deletion or insertion, preferably a substitution) at one or more positions in a region selected from the group consisting of: (i) region 7 corresponding to amino acids 1 to 153 of SEQ ID NO:6; (ii) region 8 corresponding to amino acids 177 to 613 of SEQ ID NO:6; (iii) region 9 corresponding to amino acids 659 to 730 of SEQ ID NO:6; (iv) region 10 corresponding to amino acids 804 to 806 of SEQ ID NO:6; (v) region 11 corresponding to amino acids 847 to 871 of SEQ ID NO:6; (vi) region 12 corresponding to amino acids 886 to 902 of SEQ ID NO:6; (vii) region 13 corresponding to amino acids 1005 to 1037 of SEQ ID NO:6.

In one embodiment, the detergent compositions comprise a xanthan lyase variant, comprising an alteration (e.g. a substitution, deletion or insertion, preferably a substitution) at one or more positions in two or more regions selected from the group consisting of: region 7 corresponding to amino acids 1 to 153 of SEQ ID NO:6, region 8 corresponding to amino acids 177 to 613 of SEQ ID NO:6, region 9 corresponding to amino acids 659 to 730 of SEQ ID NO:6, region 10 corresponding to amino acids 804 to 806 of SEQ ID NO:6, region 11 corresponding to amino acids 847 to 871 of SEQ ID NO:6, region 12 corresponding to amino acids 886 to 902 of SEQ ID NO:6, and region 13 corresponding to amino acids 1005 to 1037 of SEQ ID NO:6, wherein said variant has at least 60% and less than 100% sequence identity to SEQ ID NO:6; preferably said xanthan lyase variant has activity on xanthan gum, further preferably said activity is a xanthan gum degrading activity.

In one embodiment, the detergent compositions comprise a xanthan lyase variant as described herein having multiple alterations (such as 2, 3, 4, 5, 6, 7, 8, 9 or 10) in one region (e.g. of SEQ ID NO:6 or another parent xanthan lyase) selected from the group consisting of: region 7 corresponding to amino acids 1 to 153 of SEQ ID NO:6, region 8 corresponding to amino acids 177 to 613 of SEQ ID NO:6, region 9 corresponding to amino acids 659 to 730 of SEQ ID NO:6, region 10 corresponding to amino acids 804 to 806 of SEQ ID NO:6, region 11 corresponding to amino acids 847 to 871 of SEQ ID NO:6, region 12 corresponding to amino acids 886 to 902 of SEQ ID NO:6, and region 13 corresponding to amino acids 1005 to 1037 of SEQ ID NO:6, wherein said variant has at least 60% and less than 100% sequence identity to SEQ ID NO:6, preferably said variant has activity on xanthan gum, further preferably said activity is a xanthan gum degrading activity.

In one embodiment, the detergent compositions comprise a xanthan lyase variant as described herein having multiple alterations (e.g. 2, 3, 4, 5, 6, 7, 8, 9 or 10) in multiple regions (e.g. 2, 3, 4, 5, 6 or 7) (e.g. of SEQ ID NO:6 or another parent xanthan lyase) selected from the group consisting of: region 7 corresponding to amino acids 1 to 153 of SEQ ID NO:6, region 8 corresponding to amino acids 177 to 613 of SEQ ID NO:6, region 9 corresponding to amino acids 659 to 730 of SEQ ID NO:6, region 10 corresponding to amino acids 804 to 806 of SEQ ID NO:6, region 11 corresponding to amino acids 847 to 871 of SEQ ID NO:6, region 12 corresponding to amino acids 886 to 902 of SEQ ID NO:6, and region 13 corresponding to amino acids 1005 to 1037 of SEQ ID NO:6, wherein said variant has at least 60% and less than 100% sequence identity to SEQ ID NO:6, preferably said variant has activity on xanthan gum, further preferably said activity is a xanthan gum degrading activity.

In one embodiment, the detergent composition comprises xanthan lyase variants, comprising an alteration (e.g. a substitution, deletion or insertion, preferably a substitution) at one or more positions of the mature parent polypeptide (e.g. SEQ ID NO:6), wherein each alteration is independently a substitution, insertion or deletion, wherein the variant has xanthan lyase activity.

In an embodiment, the variant has sequence identity of at least 60%, e.g. at least 65%, 70%, 75%, 80%, 85%, 90%, 91%, 92%, 93%, 94%, 95%, 96%, 97%, 98%, or 99%, but less than 100%, to the amino acid sequence of the parent xanthan lyase.

In one embodiment, the variant has at least 60%, e.g. at least 65%, 70%, 75%, 80%, 85%, 90%, 91%, 92%, 93%, 94%, 95%, 96%, 97%, 98%, or 99%, but less than 100%, sequence identity to the mature polypeptide of SEQ ID NO:6.

In one embodiment, the detergent composition comprises a xanthan lyase variant as described herein, having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:6.

In another aspect, a variant comprises an alteration at one or more positions corresponding to positions 155, 159, 620, 624, 626, 631, 635, 645, 649, 650, 656, 738, 745, 746, 748, 752, 753, 754, 757, 764, 769, 774, 775, 777, 779, 782, 785, 786, 789, 792, 796, 799, 800, 801, 819, 824, 843, 845, 875, 903, 911, 912, 915, 919, 921, 923, 925, 927, 928, 930, 932, 933, 941, 966, 967, 991 and 998. In another aspect, a variant comprises an alteration at two positions corresponding to any of positions 155, 159, 620, 624, 626, 631, 635, 645, 649, 650, 656, 738, 745, 746, 748, 752, 753, 754, 757, 764, 769, 774, 775, 777, 779, 782, 785, 786, 789, 792, 796, 799, 800, 801, 819, 824, 843, 845, 875, 903, 911, 912, 915, 919, 921, 923, 925, 927, 928, 930, 932, 933, 941, 966, 967, 991 and 998. In another aspect, a variant comprises an alteration at three positions corresponding to any of positions 155, 159, 620, 624, 626, 631, 635, 645, 649, 650, 656, 738, 745, 746, 748, 752, 753, 754, 757, 764, 769, 774, 775, 777, 779, 782, 785, 786, 789, 792, 796, 799, 800, 801, 819, 824, 843, 845, 875, 903, 911, 912, 915, 919, 921, 923, 925, 927, 928, 930, 932, 933, 941, 966, 967, 991 and 998. In another aspect, a variant comprises an alteration at four or more positions, e.g. five, six, seven, eight, nine, ten or more positions, corresponding to positions 155, 159, 620, 624, 626, 631, 635, 645, 649, 650, 656, 738, 745, 746, 748, 752, 753, 754, 757, 764, 769, 774, 775, 777, 779, 782, 785, 786, 789, 792, 796, 799, 800, 801, 819, 824, 843, 845, 875, 903, 911, 912, 915, 919, 921, 923, 925, 927, 928, 930, 932, 933, 941, 966, 967, 991 and 998.

In one embodiment, the detergent composition comprises a xanthan lyase variant, having an alteration at one or more positions selected from the group consisting of positions: 155, 159, 620, 624, 626, 631, 635, 645, 649, 650, 656, 738, 745, 746, 748, 752, 753, 754, 757, 764, 769, 774, 775, 777, 779, 782, 785, 786, 789, 792, 796, 799, 800, 801, 819, 824, 843, 845, 875, 903, 911, 912, 915, 919, 921, 923, 925, 927, 928, 930, 932, 933, 941, 966, 967, 991 and 998 of SEQ ID NO:6, wherein each position corresponds to the positions of SEQ ID NO:6.

In another aspect, a variant comprises an alteration at one or more positions corresponding to positions 9, 15, 46, 58, 66, 89, 95, 100, 106, 109, 183, 188, 190, 203, 204, 221, 229, 234, 238, 240, 242, 243, 257, 258, 291, 293, 316, 320, 324, 329, 333, 339, 341, 352, 354, 360, 377, 400, 419, 450, 451, 454, 481, 492, 567, 568, 578, 579, 664, 672, 855, 887 and 892. In another aspect, a variant comprises an alteration at two positions corresponding to any of positions 9, 15, 46, 58, 66, 89, 95, 100, 106, 109, 183, 188, 190, 203, 204, 221, 229, 234, 238, 240, 242, 243, 257, 258, 291, 293, 316, 320, 324, 329, 333, 339, 341, 352, 354, 360, 377, 400, 419, 450, 451, 454, 481, 492, 567, 568, 578, 579, 664, 672, 855, 887 and 892. In another aspect, a variant comprises an alteration at three positions corresponding to any of positions 9, 15, 46, 58, 66, 89, 95, 100, 106, 109, 183, 188, 190, 203, 204, 221, 229, 234, 238, 240, 242, 243, 257, 258, 291, 293, 316, 320, 324, 329, 333, 339, 341, 352, 354, 360, 377, 400, 419, 450, 451, 454, 481, 492, 567, 568, 578, 579, 664, 672, 855, 887 and 892. In another aspect, a variant comprises an alteration at four or more positions, e.g. five, six, seven, eight, nine, ten or more positions, corresponding to positions 9, 15, 46, 58, 66, 89, 95, 100, 106, 109, 183, 188, 190, 203, 204, 221, 229, 234, 238, 240, 242, 243, 257, 258, 291, 293, 316, 320, 324, 329, 333, 339, 341, 352, 354, 360, 377, 400, 419, 450, 451, 454, 481, 492, 567, 568, 578, 579, 664, 672, 855, 887 and 892.

In one embodiment, the detergent composition comprises a xanthan lyase variant, having an alteration at one or more positions selected from the group consisting of: Y155E, A159P, K620R, A624E, A626G, T631N, T631E, S635E, S635T, S635Q, A645S, T649V, T649K, T649R, Q650G, I656V, G738L, K745R, F746L, L748T, P752R, P752K, G753E, G753Q, G753S, S754E, S754L, S754Q, S754R, S757D, S757P, S757E, P764V, P764K, A769D, A769T, A769R, A769S, A769E, A769Q, A769*, A774V, L775M, L775Y, L775A, L7751, L7755, L775F, L775Q, D777K, D777R, P779V, Y7821, A785T, N786K, G789R, K792W, K792Y, K792V, K792A, N796Q, A799H, V800P, D801G, K819R, K819T, K824R, A843P, D845E, K875T, K875E, T903A, T903Q, A911V, A911M, A911S, A912T, A9121, A912Y, T915Q, T915S, T915V, T915A, T919F, T919G, T919D, T921R, T921S, T923H, T923D, T925Q, T925D, T925R, T927K, D928W, Y930H, Y930L, Y930F, A932P, D933M, G941E, G941D, A966P, A967D, N991D and V998K., wherein numbering is according to SEQ ID NO:6.

In another aspect, the variant comprises or consists of an alteration at a position corresponding to position 155. In another aspect, the amino acid at a position corresponding to position 155 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution Y155E of the mature polypeptide of SEQ ID NO:6.

In another aspect, the variant comprises or consists of an alteration at a position corresponding to position 159. In another aspect, the amino acid at a position corresponding to position 159 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A159P.

In another aspect, the variant comprises or consists of an alteration at a position corresponding to position 620. In another aspect, the amino acid at a position corresponding to position 620 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K620R of the mature polypeptide of SEQ ID NO:6.

In another aspect, the variant comprises or consists of an alteration at a position corresponding to position 624. In another aspect, the amino acid at a position corresponding to position 624 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A624E of the mature polypeptide of SEQ ID NO:6

In another aspect, the variant comprises or consists of an alteration at a position corresponding to position 626. In another aspect, the amino acid at a position corresponding to position 626 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A626Q of the mature polypeptide of SEQ ID NO:6.

In another aspect, the variant comprises or consists of an alteration at a position corresponding to position 631. In another aspect, the amino acid at a position corresponding to position 631 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T631N or T631E of the mature polypeptide of SEQ ID NO:6. A preferred substitution at a position corresponding to position 631 is T631N.

In another aspect, the variant comprises or consists of an alteration at a position corresponding to position 635. In another aspect, the amino acid at a position corresponding to position 635 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution S635E, S635T or S635Q. A preferred substitution at a position corresponding to position 635 is S635E.

In another aspect, the variant comprises or consists of an alteration at a position corresponding to position 649. In another aspect, the amino acid at a position corresponding to position 649 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution T649V, T649K or T649R of the mature polypeptide of SEQ ID NO:6. A preferred substitution at a position corresponding to position 649 is T649K.

In another aspect, the variant comprises or consists of an alteration at a position corresponding to position 650. In another aspect, the amino acid at a position corresponding to position 650 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution Q650G of the mature polypeptide of SEQ ID NO:6.

In another aspect, the variant comprises or consists of an alteration at a position corresponding to position 656. In another aspect, the amino acid at a position corresponding to position 656 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution I656V of the mature polypeptide of SEQ ID NO:6.

In another aspect, the variant comprises or consists of an alteration at a position corresponding to position 738. In another aspect, the amino acid at a position corresponding to position 738 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution G738L of the mature polypeptide of SEQ ID NO:6.

In another aspect, the variant comprises or consists of an alteration at a position corresponding to position 745. In another aspect, the amino acid at a position corresponding to position 745 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K745R of the mature polypeptide of SEQ ID NO:6.

In another aspect, the variant comprises or consists of an alteration at a position corresponding to position 746. In another aspect, the amino acid at a position corresponding to position 746 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution F746L of the mature polypeptide of SEQ ID NO:6.

In another aspect, the variant comprises or consists of an alteration at a position corresponding to position 748. In another aspect, the amino acid at a position corresponding to position 748 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution L748T of the mature polypeptide of SEQ ID NO:6.

In another aspect, the variant comprises or consists of an alteration at a position corresponding to position 752. In another aspect, the amino acid at a position corresponding to position 752 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution P752R or P752K of the mature polypeptide of SEQ ID NO:6.

In another aspect, the variant comprises or consists of an alteration at a position corresponding to position 753. In another aspect, the amino acid at a position corresponding to position 753 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution G753E, G753Q or G753S of the mature polypeptide of SEQ ID NO:6. A preferred substitution at a position corresponding to position 753 is G753E.

In another aspect, the variant comprises or consists of an alteration at a position corresponding to position 754. In another aspect, the amino acid at a position corresponding to position 754 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution S754E, S754L, S754Q or S754R of the mature polypeptide of SEQ ID NO:6. A preferred substitution at a position corresponding to position 754 is S754E or S754R.

In another aspect, the variant comprises or consists of an alteration at a position corresponding to position 757. In another aspect, the amino acid at a position corresponding to position 757 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution S757D, S757P or S757E of the mature polypeptide of SEQ ID NO:6. A preferred substitution at a position corresponding to position 757 is S757D.

In another aspect, the variant comprises or consists of an alteration at a position corresponding to position 764. In another aspect, the amino acid at a position corresponding to position 764 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution P764V or P764K of the mature polypeptide of SEQ ID NO:6. A preferred substitution at a position corresponding to position 764 is P764V.

In another aspect, the variant comprises or consists of an alteration at a position corresponding to position 769. In another aspect, the amino acid at a position corresponding to position 769 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the alteration A769D, A769T, A769R, A769S, A769E, A769Q or A769* of the mature polypeptide of SEQ ID NO:6. A preferred substitution at a position corresponding to position 769 is A769D.

In another aspect, the variant comprises or consists of an alteration at a position corresponding to position 774. In another aspect, the amino acid at a position corresponding to position 774 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A774V of the mature polypeptide of SEQ ID NO:6.

In another aspect, the variant comprises or consists of an alteration at a position corresponding to position 775. In another aspect, the amino acid at a position corresponding to position 775 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution L775A or L775F or L775I or L775M or L775Q or L7755 or L775Y of the mature polypeptide of SEQ ID NO:6. A preferred substitution at a position corresponding to position 775 is L775M, L775Y or L775A.

In another aspect, the variant comprises or consists of an alteration at a position corresponding to position 779. In another aspect, the amino acid at a position corresponding to position 779 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution P779V of the mature polypeptide of SEQ ID NO:6.

In another aspect, the variant comprises or consists of an alteration at a position corresponding to position 782. In another aspect, the amino acid at a position corresponding to position 782 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution Y782I.

In another aspect, the variant comprises or consists of an alteration at a position corresponding to position 786. In another aspect, the amino acid at a position corresponding to position 786 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution N786K.

In another aspect, the variant comprises or consists of an alteration at a position corresponding to position 789. In another aspect, the amino acid at a position corresponding to position 789 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution G789R.

In another aspect, the variant comprises or consists of an alteration at a position corresponding to position 792. In another aspect, the amino acid at a position corresponding to position 792 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K792W, K792Y, K792V or K792A of the mature polypeptide of SEQ ID NO:6. A preferred substitution at a position corresponding to position 792 is K792W or K792Y.

In another aspect, the variant comprises or consists of an alteration at a position corresponding to position 796. In another aspect, the amino acid at a position corresponding to position 796 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution N796Q of the mature polypeptide of SEQ ID NO:6.

In another aspect, the variant comprises or consists of an alteration at a position corresponding to position 799. In another aspect, the amino acid at a position corresponding to position 799 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A799H of the mature polypeptide of SEQ ID NO:6.

In another aspect, the variant comprises or consists of an alteration at a position corresponding to position 800. In another aspect, the amino acid at a position corresponding to position 800 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution V800P of the mature polypeptide of SEQ ID NO:6.

In another aspect, the variant comprises or consists of an alteration at a position corresponding to position 801. In another aspect, the amino acid at a position corresponding to position 801 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution D801G of the mature polypeptide of SEQ ID NO:6.

In another aspect, the variant comprises or consists of an alteration at a position corresponding to position 819. In another aspect, the amino acid at a position corresponding to position 819 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K819R or K819T of the mature polypeptide of SEQ ID NO:6. A preferred substitution at a position corresponding to position 819 is K819R or K819T.

In another aspect, the variant comprises or consists of an alteration at a position corresponding to position 824. In another aspect, the amino acid at a position corresponding to position 824 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K824R of the mature polypeptide of SEQ ID NO:6.

In another aspect, the variant comprises or consists of an alteration at a position corresponding to position 843. In another aspect, the amino acid at a position corresponding to position 843 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A843P.

In another aspect, the variant comprises or consists of an alteration at a position corresponding to position 845. In another aspect, the amino acid at a position corresponding to position 845 is substituted with Ala, Arg, Asn, Asp, Cys, Gln, Glu, Gly, His, Ile, Leu, Lys, Met, Phe, Pro, Ser, Thr, Trp, Tyr, or Val. A preferred substitution at a position corresponding to position 845 is D845E.

In another aspect, the variant comprises or consists of an alteration at a position corresponding to position 875. In another aspect, the amino acid at a position corresponding to position 875 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution K875T or K875E of the mature polypeptide of SEQ ID NO:6. A preferred substitution at a position corresponding to position 875 is K875T.

In another aspect, the variant comprises or consists of an alteration at a position corresponding to position 903. In another aspect, the amino acid at a position corresponding to position 903 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution T903A or T903Q of the mature polypeptide of SEQ ID NO:6.

In another aspect, the variant comprises or consists of an alteration at a position corresponding to position 911. In another aspect, the amino acid at a position corresponding to position 911 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A911M, A911V or A911S of the mature polypeptide of SEQ ID NO:6. A preferred substitution at a position corresponding to position 911 is A911V.

In another aspect, the variant comprises or consists of an alteration at a position corresponding to position 912. In another aspect, the amino acid at a position corresponding to position 912 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A912I or A912T or A912Y of the mature polypeptide of SEQ ID NO:6. A preferred substitution at a position corresponding to position 912 is A912T.

In another aspect, the variant comprises or consists of an alteration at a position corresponding to position 915. In another aspect, the amino acid at a position corresponding to position 915 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T915S, T915Q, T915A or T915V of the mature polypeptide of SEQ ID NO:6.

In another aspect, the variant comprises or consists of an alteration at a position corresponding to position 919. In another aspect, the amino acid at a position corresponding to position 919 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution T919D, T919F or T919G of the mature polypeptide of SEQ ID NO:6.

In another aspect, the variant comprises or consists of an alteration at a position corresponding to position 921. In another aspect, the amino acid ata position corresponding to position 921 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T921R or T921S of the mature polypeptide of SEQ ID NO:6.

In another aspect, the variant comprises or consists of an alteration at a position corresponding to position 923. In another aspect, the amino acid at a position corresponding to position 923 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T923D or T923H of the mature polypeptide of SEQ ID NO:6.

In another aspect, the variant comprises or consists of an alteration at a position corresponding to position 925. In another aspect, the amino acid at a position corresponding to position 925 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T925D or T925Q or T925R of the mature polypeptide of SEQ ID NO:6.

In another aspect, the variant comprises or consists of an alteration at a position corresponding to position 927. In another aspect, the amino acid at a position corresponding to position 927 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution T927K of the mature polypeptide of SEQ ID NO:6.

In another aspect, the variant comprises or consists of an alteration at a position corresponding to position 928. In another aspect, the amino acid at a position corresponding to position 928 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution D928W of the mature polypeptide of SEQ ID NO:6.

In another aspect, the variant comprises or consists of an alteration at a position corresponding to position 930. In another aspect, the amino acid at a position corresponding to position 930 is substituted with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution Y930F or Y930H or Y930L of the mature polypeptide of SEQ ID NO:6.

In another aspect, the variant comprises or consists of an alteration at a position corresponding to position 933. In another aspect, the amino acid at a position corresponding to position 933 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution D933M of the mature polypeptide of SEQ ID NO:6.

In another aspect, the variant comprises or consists of an alteration at a position corresponding to position 941. In another aspect, the amino acid at a position corresponding to position 941 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution G941D or G941E of the mature polypeptide of SEQ ID NO:6.

In another aspect, the variant comprises or consists of an alteration at a position corresponding to position 966. In another aspect, the amino acid at a position corresponding to position 966 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A966P of the mature polypeptide of SEQ ID NO:6.

In another aspect, the variant comprises or consists of an alteration at a position corresponding to position 991. In another aspect, the amino acid at a position corresponding to position 991 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution N991D of the mature polypeptide of SEQ ID NO:6.

In another aspect, the variant comprises or consists of an alteration at a position corresponding to position 998. In another aspect, the amino acid at a position corresponding to position 998 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution V998K of the mature polypeptide of SEQ ID NO:6.

In one aspect, the variant comprises or consists of an alteration at a position corresponding to position 9. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution K9R of the mature polypeptide of SEQ ID NO:6.

In one aspect, the variant comprises or consists of an alteration at a position corresponding to position 15. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution N15T of the mature polypeptide of SEQ ID NO:6.

In one aspect, the variant comprises or consists of an alteration at a position corresponding to position 46. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution L46D of the mature polypeptide of SEQ ID NO:6.

In one aspect, the variant comprises or consists of an alteration at a position corresponding to position 58. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution A58L of the mature polypeptide of SEQ ID NO:6.

In one aspect, the variant comprises or consists of an alteration at a position corresponding to position 66. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution S66H of the mature polypeptide of SEQ ID NO:6.

In one aspect, the variant comprises or consists of an alteration at a position corresponding to position 89. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution Q89Y of the mature polypeptide of SEQ ID NO:6.

In one aspect, the variant comprises or consists of an alteration at a position corresponding to position 95. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution K95E of the mature polypeptide of SEQ ID NO:6.

In one aspect, the variant comprises or consists of an alteration at a position corresponding to position 100. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution S100D of the mature polypeptide of SEQ ID NO:6.

In one aspect, the variant comprises or consists of an alteration at a position corresponding to position 106. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution N106Y of the mature polypeptide of SEQ ID NO:6.

In one aspect, the variant comprises or consists of an alteration at a position corresponding to position 109. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution Q109R, Q109D, Q109F, Q109K or Q109A of the mature polypeptide of SEQ ID NO:6. A preferred substitution at a position corresponding to position 109 is Q109R.

In one aspect, the variant comprises or consists of an alteration at a position corresponding to position 183. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution K183Q or K183R of the mature polypeptide of SEQ ID NO:6.

In one aspect, the variant comprises or consists of an alteration at a position corresponding to position 188. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution V1881 of the mature polypeptide of SEQ ID NO:6.

In one aspect, the variant comprises or consists of an alteration at a position corresponding to position 190. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution Al 90Q of the mature polypeptide of SEQ ID NO:6.

In one aspect, the variant comprises or consists of an alteration at a position corresponding to position 203. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution A203P of the mature polypeptide of SEQ ID NO:6.

In one aspect, the variant comprises or consists of an alteration at a position corresponding to position 204. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution K204R of the mature polypeptide of SEQ ID NO:6.

In one aspect, the variant comprises or consists of an alteration at a position corresponding to position 221. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution A221P of the mature polypeptide of SEQ ID NO:6.

In one aspect, the variant comprises or consists of an alteration at a position corresponding to position 229. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution E229N or E229S of the mature polypeptide of SEQ ID NO:6.

In one aspect, the variant comprises or consists of an alteration at a position corresponding to position 234. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution I234V of the mature polypeptide of SEQ ID NO:6.

In one aspect, the variant comprises or consists of an alteration at a position corresponding to position 238. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution I238W, I238L or I238M of the mature polypeptide of SEQ ID NO:6. Preferred substitutions at a position corresponding to position 238 are I238W and I238L.

In one aspect, the variant comprises or consists of an alteration at a position corresponding to position 240. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution 1240W of the mature polypeptide of SEQ ID NO:6.

In one aspect, the variant comprises or consists of an alteration at a position corresponding to position 242. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution N242S of the mature polypeptide of SEQ ID NO:6.

In one aspect, the variant comprises or consists of an alteration at a position corresponding to position 243. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution G243V of the mature polypeptide of SEQ ID NO:6.

In one aspect, the variant comprises or consists of an alteration at a position corresponding to position 257. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution Y257W of the mature polypeptide of SEQ ID NO:6.

In one aspect, the variant comprises or consists of an alteration at a position corresponding to position 258. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution R258E of the mature polypeptide of SEQ ID NO:6.

In one aspect, the variant comprises or consists of an alteration at a position corresponding to position 291. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution K291R of the mature polypeptide of SEQ ID NO:6.

In one aspect, the variant comprises or consists of an alteration at a position corresponding to position 293. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution A293G or A293P of the mature polypeptide of SEQ ID NO:6.

In one aspect, the variant comprises or consists of an alteration at a position corresponding to position 316. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution K316R of the mature polypeptide of SEQ ID NO:6.

In one aspect, the variant comprises or consists of an alteration at a position corresponding to position 320. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution K320R of the mature polypeptide of SEQ ID NO:6.

In one aspect, the variant comprises or consists of an alteration at a position corresponding to position 324. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution L324Q of the mature polypeptide of SEQ ID NO:6.

In one aspect, the variant comprises or consists of an alteration at a position corresponding to position 329. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution K329R of the mature polypeptide of SEQ ID NO:6.

In one aspect, the variant comprises or consists of an alteration at a position corresponding to position 333. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution K333R of the mature polypeptide of SEQ ID NO:6.

In one aspect, the variant comprises or consists of an alteration at a position corresponding to position 339. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution L339M of the mature polypeptide of SEQ ID NO:6.

In one aspect, the variant comprises or consists of an alteration at a position corresponding to position 341. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution 1341P of the mature polypeptide of SEQ ID NO:6.

In one aspect, the variant comprises or consists of an alteration at a position corresponding to position 352. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution V3521 of the mature polypeptide of SEQ ID NO:6.

In one aspect, the variant comprises or consists of an alteration at a position corresponding to position 354. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution S354P of the mature polypeptide of SEQ ID NO:6.

In one aspect, the variant comprises or consists of an alteration at a position corresponding to position 360. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution K360R of the mature polypeptide of SEQ ID NO:6.

In one aspect, the variant comprises or consists of an alteration at a position corresponding to position 377. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution F377Y of the mature polypeptide of SEQ ID NO:6.

In one aspect, the variant comprises or consists of an alteration at a position corresponding to position 400. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution K400R of the mature polypeptide of SEQ ID NO:6.

In one aspect, the variant comprises or consists of an alteration at a position corresponding to position 419. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution F419Y of the mature polypeptide of SEQ ID NO:6.

In one aspect, the variant comprises or consists of an alteration at a position corresponding to position 450. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution D450P of the mature polypeptide of SEQ ID NO:6.

In one aspect, the variant comprises or consists of an alteration at a position corresponding to position 451. In one embodiment, the amino acid at this position may substituted with Ala, Arg, Asn, Asp, Cys, Gin, Glu, Gly, His, Ile, Leu, Lys, Met, Phe, Pro, Ser, Thr, Trp, Tyr, or Val. In a particular embodiment, the variant comprises or consists of the substitution K451E or K451R of the mature polypeptide of SEQ ID NO:6.

In one aspect, the variant comprises or consists of an alteration at a position corresponding to position 454. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution A454V of the mature polypeptide of SEQ ID NO:6.

In one aspect, the variant comprises or consists of an alteration at a position corresponding to position 481. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution K481R of the mature polypeptide of SEQ ID NO:6.

In one aspect, the variant comprises or consists of an alteration at a position corresponding to position 492. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution A492L of the mature polypeptide of SEQ ID NO:6.

In one aspect, the variant comprises or consists of an alteration at a position corresponding to position 567. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution K567R of the mature polypeptide of SEQ ID NO:6.

In one aspect, the variant comprises or consists of an alteration at a position corresponding to position 568. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution G568A of the mature polypeptide of SEQ ID NO:6.

In one aspect, the variant comprises or consists of an alteration at a position corresponding to position 578. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution S578K or S578R of the mature polypeptide of SEQ ID NO:6.

In one aspect, the variant comprises or consists of an alteration at a position corresponding to position 579. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution S579R or S579K of the mature polypeptide of SEQ ID NO:6. A preferred substitution at a position corresponding to position 579 is S579R.

In one aspect, the variant comprises or consists of an alteration at a position corresponding to position 664. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution T664K of the mature polypeptide of SEQ ID NO:6.

In one aspect, the variant comprises or consists of an alteration at a position corresponding to position 672. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution N672D of the mature polypeptide of SEQ ID NO:6.

In one aspect, the variant comprises or consists of an alteration at a position corresponding to position 885. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution K855R of the mature polypeptide of SEQ ID NO:6.

In one aspect, the variant comprises or consists of an alteration at a position corresponding to position 887. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution K887R of the mature polypeptide of SEQ ID NO:6.

In one aspect, the variant comprises or consists of an alteration at a position corresponding to position 892. In one embodiment, the amino acid at this position may substituted with Ala, Arg, Asn, Asp, Cys, Gln, Glu, Gly, His, Ile, Leu, Lys, Met, Phe, Pro, Ser, Thr, Trp, Tyr, or Val. In a particular embodiment, the variant comprises or consists of the substitution N892Y, N892W or N892F of the mature polypeptide of SEQ ID NO:6. A preferred substitution at a position corresponding to position 892 is N892Y.

In one embodiment the present invention relates to detergent compositions comprising a xanthan lyase variant as described herein, having an alteration at one or more positions selected from the group consisting of positions: 9, 15, 46, 58, 66, 89, 95, 100, 106, 109, 183, 188, 190, 203, 204, 221, 229, 234, 238, 240, 242, 243, 257, 258, 291, 293, 316, 320, 324, 329, 333, 339, 341, 352, 354, 360, 377, 400, 419, 450, 451, 454, 481, 492, 567, 568, 578, 579, 664, 672, 855, 887 and 892 of SEQ ID NO:6, wherein each position corresponds to the positions of SEQ ID NO:6.

In one embodiment the present invention relates to detergent compositions comprising a xanthan lyase variant as described herein having one or more substitutions selected from the group consisting of: K9R, N15T, L46D, A58L, S66H, Q89Y, K95E, S100D, N106Y, Q109R, Q109D, Q109F, Q109K, Q109A, K183Q,K183R, V188I, A190Q, A203P, K204R, A221P, E229N, E229S, I234V, I238W, I238L, I238M, I240W, N242S, G243V, Y257W, R258E, K291R, A293G, A293P, K316R, K320R, L324Q, K329R, K333R, L339M, I341P, V352I, S354P, K360R, F377Y, K400R, F419Y, D450P, K451E, K451R, A454V, K481R, A492L, K567R, G568A, S578K, S578R, S579R, S579K, T664K, N672D, K855R, K887R, N892Y, N892W and N892F, wherein numbering is according to SEQ ID NO:6.

Preferred are xanthan lyase variants having an alteration (e.g. a substitution, deletion or insertion, preferably a substitution) in the positions selected from the group consisting of positions: 190, 229, 234, 440, 582, 624, 631, 635, 672, 703, 738, 752, 753, 754, 757, 769, 775, 801, 875, 892, and any combination thereof, preferably 229+672+752+753+769+775+801+875+892; 229+672+753+754+769+775+801+875+892;229+672+752+753+754+769+775+801+875+892; 190+229+234+624+672+753+754+769+775+801+875; 190+229+631+672+703+752+753+769+775+801+875; 190+229+234+582+672+753+754+757+769+775+801+875+892; or 229+440+582+624+635+672+738+753+754+757+769+775+801+875+892 of SEQ ID NO:6.

Particularly preferred are xanthan lyase variants having an alteration (e.g. a substitution, deletion or insertion, preferably a substitution) selected from the group consisting of the following alterations: E229N+N672D+P752K+G753E+A769D+L775A+D801G+K875T+N892Y; E2295+N672D+G753E+5754E+A769D+L775A+D801G+K875T+N892Y; E2295+N672D+P752R+G753E+S754E+A769D+L775A+D801G+K875T+N892Y; A190Q+E229S+I234V+A624E+N672D+G753E+S754E+A769D+L775A+D801G+K875T; A190Q+E229S+T631N+N672D+1703L+P752K+G753E+A769D+L775A+D801G+K875T; A190Q+E229S+1234V+S582K+N672D+G753E+S754E+S757D+A769D+L775A+D801G+K875T+N892Y; E229S+N440K+S582K+A624E+S635E+N672D+G738L+G753E+S754E+S757D+A769D+L775A+D80 1G+K875T+N892Y; and S100D+E229S+K360G+D458S+S582K+N672D+G753E+S754E+S757D+A769D+L775A+D801G+A8 43P+K875T+N892Y+N1008D.

All of the above alterations are relative to SEQ ID NO:6.

In a particular embodiment, the invention relates to a detergent composition comprising a xanthan lyase variant selected from the group consisting of the xanthan lyase variants set forth in Table 17 herein.

In a particular embodiment, the invention relates to a detergent composition comprising a xanthan lyase variant selected from the group consisting of the xanthan lyase variants set forth in Table 18 herein.

In a particular embodiment, the invention relates to a detergent composition comprising a xanthan lyase variant selected from the group consisting of the xanthan lyase variants set forth in Table 19 herein.

In a particular embodiment, the invention relates to a detergent composition comprising a xanthan lyase variant selected from the group consisting of the xanthan lyase variants set forth in Table 20 herein.

In a particular embodiment, the invention relates to a detergent composition comprising a xanthan lyase variant selected from the group consisting of the xanthan lyase variants set forth in Table 21 herein.

In a particular embodiment, the invention relates to a detergent composition comprising a xanthan lyase variant selected from the group consisting of the xanthan lyase variants set forth in Table 22 herein.

In a particular embodiment, the invention relates to a detergent composition comprising a xanthan lyase variant selected from the group consisting of the xanthan lyase variants set forth in Table 23 herein.

In a particular embodiment, the invention relates to a detergent composition comprising a xanthan lyase variant selected from the group consisting of the xanthan lyase variants set forth in Table 24 herein.

In a particular embodiment, the invention relates to a detergent composition comprising a xanthan lyase variant selected from the group consisting of the xanthan lyase variants set forth in Table 25 herein.

In a particular embodiment, the invention relates to a detergent composition comprising a xanthan lyase variant selected from the group consisting of the xanthan lyase variants set forth in Table 26 herein.

In a particular embodiment, the invention relates to a detergent composition comprising a xanthan lyase variant selected from the group consisting of the xanthan lyase variants set forth in Table 27 herein.

In a particular embodiment, the invention relates to a detergent composition comprising a xanthan lyase variant selected from the group consisting of the xanthan lyase variants set forth in Table 28 herein.

In a particular embodiment, the invention relates to a detergent composition comprising a xanthan lyase variant selected from the group consisting of the xanthan lyase variants set forth in Table 29 herein.

In a particular embodiment, the invention relates to detergent compositions comprising a xanthan lyase variant selected from the group consisting of the xanthan lyase variants set forth in Table 30 herein.

In a particular embodiment, the invention relates to detergent compositions comprising a xanthan lyase variant selected from the group consisting of the xanthan lyase variants set forth in Table 31 herein.

In a particular embodiment, the invention relates to detergent compositions comprising a xanthan lyase variant selected from the group consisting of the xanthan lyase variants set forth in Table 32 herein.

In a particular embodiment, the invention relates to detergent compositions comprising a xanthan lyase variant selected from the group consisting of the xanthan lyase variants set forth in Table 33 herein.

In various embodiments, the preferred endoglucanase variants are combined with the preferred xanthan lyase variants. In some aspects, the detergent composition thus comprises

(A) an endoglucanase variant selected from those having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% sequence identity to SEQ ID NO:2 and having an alteration (e.g. a substitution, deletion or insertion, preferably a substitution) in the positions selected from the group consisting of positions: 559+579+697; 512+559+579+697; 18+71+186+408+579+602+651+688+756; 18+189+408+559+579+688+697+756+921+934; 313+488; 880+905+921+934; 302+313+408+579+602+651+697+880+921+934; or 216+313+408+476+579+602+638+651+697+719+880+887+921+934 of SEQ ID NO:2, preferably having an alteration selected from the group consisting of the following alterations: (A1) A559N+Y579W+T697G; (A2) K512P+A559N+Y579W+T697G; (A3) N18G+A71E+A186P+E408D+Y579W+1602T+A651P+A688G+V756Y; (A4) N18G+N189K+E408D+A559N+Y579W+A688G+T697G+V756Y+K921R+Y934G; (A5) S313D+E408D; (A6) R880K+N905D+K921R+Y934G; (A7) I302D+S313D+E408D+Y579W+I602T+A651P+T697G+R880K+K921R+Y934G; and (A8) N216Q+S313D+E408D+D476R+Y579W+I602T+F638N+A651P+T697G+W719R+R880K+T887K+K9 21R+Y934G of SEQ ID NO:2; and

(B) a xanthan lyase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% sequence identity to SEQ ID NO:6 and having an alteration (e.g. a substitution, deletion or insertion, preferably a substitution) in the positions selected from the group consisting of positions: 229+672+752+753+769+775+801+875+892; 229+672+753+754+769+775+801+875+892;229+672+752+753+754+769+775+801+875+892; 190+229+234+624+672+753+754+769+775+801+875; 190+229+631+672+703+752+753+769+775+801+875; 190+229+234+582+672+753+754+757+769+775+801+875+892; 229+440+582+624+635+672+738+753+754+757+769+775+801+875+892; or 100+229+360+458+582+672+753+754+757+769+775+801+843+875+892+1008 of SEQ ID NO:6, preferably having an alteration selected from the group consisting of the following alterations: (B1) E229N+N672D+P752K+G753E+A769D+L775A+D801G+K875T+N892Y; (B2) E229S+N672D+G753E+5754E+A769D+L775A+D801G+K875T+N892Y; (B3) E229S+N672D+P752R+G753E+S754E+A769D+L775A+D801G+K875T+N892Y; (B4) A190Q+E229S+I234V+A624E+N672D+G753E+S754E+A769D+L775A+D801G+K875T; (B5) A190Q+E229S+T631N+N672D+I703L+P752K+G753E+A769D+L775A+D801G+K875T; (B6) A190Q+E229S+1234V+S582K+N672D+G753E+S754E+S757D+A769D+L775A+D801G+K875T+N89 2Y; (B7) E229S+N440K+S582K+A624E+S635E+N672D+G738L+G753E+S754E+S757D+A769D+L775A+D80 1G+K875T+N892Y; and (B8) S100D+E229S+K360G+D458S+S582K+N672D+G753E+S754E+S757D+A769D+L775A+D801G+A8 43P+K875T+N892Y+N1008D of SEQ ID NO:6.

In some aspects, the endoglucanase and/or the xanthan lyase variant do not comprise any further substitution besides those explicitly mentioned above, i.e. the remainder of the sequence is identical to that of the parent enzyme as set forth in SEQ ID NO:2 and SEQ ID NO:6, respectively.

In some aspects, in the detergent compositions of the invention the endoglucanase variant A1, as defined above, can be combined with any one of the xanthan lyase variants B1, B2, B3, B4, B5, B6, B7 and B8, as defined above, wherein the respective enzymes preferably have at least 90%, preferably at least 91%, 92%, 93%, 94%, 95%, 96% or 97% sequence identity to SEQ ID NO:2 and SEQ ID NO:6, respectively, preferably are identical to their respective parent sequence with the exception of the substitutions explicitly mentioned herein.

In some aspects, in the detergent compositions of the invention the endoglucanase variant A2, as defined above, can be combined with any one of the xanthan lyase variants B1, B2, B3, B4, B5, B6, B7 and B8, as defined above, wherein the respective enzymes preferably have at least 90%, preferably at least 91%, 92%, 93%, 94%, 95%, 96% or 97% sequence identity to SEQ ID NO:2 and SEQ ID NO:6, respectively, preferably are identical to their respective parent sequence with the exception of the substitutions explicitly mentioned herein.

In some aspects, in the detergent compositions of the invention the endoglucanase variant A3, as defined above, can be combined with any one of the xanthan lyase variants B1, B2, B3, B4, B5, B6, B7 and B8, as defined above, wherein the respective enzymes preferably have at least 90%, preferably at least 91%, 92%, 93%, 94%, 95%, 96% or 97% sequence identity to SEQ ID NO:2 and SEQ ID NO:6, respectively, preferably are identical to their respective parent sequence with the exception of the substitutions explicitly mentioned herein.

In some aspects, in the detergent compositions of the invention the endoglucanase variant A4, as defined above, can be combined with any one of the xanthan lyase variants B1, B2, B3, B4, B5, B6, B7 and B8, as defined above, wherein the respective enzymes preferably have at least 90%, preferably at least 91%, 92%, 93%, 94%, 95%, 96% or 97% sequence identity to SEQ ID NO:2 and SEQ ID NO:6, respectively, preferably are identical to their respective parent sequence with the exception of the substitutions explicitly mentioned herein.

In some aspects, in the detergent compositions of the invention the endoglucanase variant A5, as defined above, can be combined with any one of the xanthan lyase variants B1, B2, B3, B4, B5, B6, B7 and B8, as defined above, wherein the respective enzymes preferably have at least 90%, preferably at least 91%, 92%, 93%, 94%, 95%, 96% or 97% sequence identity to SEQ ID NO:2 and SEQ ID NO:6, respectively, preferably are identical to their respective parent sequence with the exception of the substitutions explicitly mentioned herein.

In some aspects, in the detergent compositions of the invention the endoglucanase variant A6, as defined above, can be combined with any one of the xanthan lyase variants B1, B2, B3, B4, B5, B6, B7 and B8, as defined above, wherein the respective enzymes preferably have at least 90%, preferably at least 91%, 92%, 93%, 94%, 95%, 96% or 97% sequence identity to SEQ ID NO:2 and SEQ ID NO:6, respectively, preferably are identical to their respective parent sequence with the exception of the substitutions explicitly mentioned herein.

In some aspects, in the detergent compositions of the invention the endoglucanase variant A7, as defined above, can be combined with any one of the xanthan lyase variants B1, B2, B3, B4, B5, B6, B7 and B8, as defined above, wherein the respective enzymes preferably have at least 90%, preferably at least 91%, 92%, 93%, 94%, 95%, 96% or 97% sequence identity to SEQ ID NO:2 and SEQ ID NO:6, respectively, preferably are identical to their respective parent sequence with the exception of the substitutions explicitly mentioned herein.

In some aspects, in the detergent compositions of the invention the endoglucanase variant A8, as defined above, can be combined with any one of the xanthan lyase variants B1, B2, B3, B4, B5, B6, B7 and B8, as defined above, wherein the respective enzymes preferably have at least 90%, preferably at least 91%, 92%, 93%, 94%, 95%, 96% or 97% sequence identity to SEQ ID NO:2 and SEQ ID NO:6, respectively, preferably are identical to their respective parent sequence with the exception of the substitutions explicitly mentioned herein.

If not explicitly indicated otherwise, all the variants described above may further comprise one or more additional alterations at one or more (e.g. several) other positions in any of the regions described herein.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R.L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, LeuNal, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for xanthan lyase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

In one embodiment, the present invention relates to a detergent composition comprising an endoglucanase variant of the invention, having the total number of alterations compared to SEQ ID NO:2 between 1 and 20, e.g. between 1 and 18 or between 5 and 15 or between 8 and 14, such as 1, 2, 3, 4, 5, 6, 7, 8, 9, 10, 11, 12, 13, 14 or 15 alterations, and a xanthan lyase variant as described herein, having a total number of alterations compared to SEQ ID NO:6 between 1 and 20, e.g.

between 1 and 18 or between 5 and 17 or between 10 and 16, such as 1, 2, 3, 4, 5, 6, 7, 8, 9, 10, 11, 12, 13, 14, 15, 16, 17 or 18 alterations.

In one embodiment, the present invention relates to a detergent composition comprising an endoglucanase variant of the invention and a xanthan lyase variant of the invention, having an activity on xanthan gum, preferably said activity on xanthan gum is a xanthan gum degrading activity, further preferably said xanthan gum degrading activity is EC 4.2.2.12 activity and endoglucanase EC 3.2.1.4 activity.

In an embodiment, the variant has an improved stability in a detergent composition compared to a parent enzyme (e.g. SEQ ID NO:2 or 6). In one embodiment, the improved stability is measured as an improved half-life. In one embodiment, the improved stability is measured as half-life improvement factor.

In one embodiment, the present invention relates to a detergent composition comprising an endoglucanase/xanthan lyase variant of the invention, wherein said variant has an improved stability in a detergent composition compared to a parent enzyme (e.g. with SEQ ID NO:2 or 6); preferably said detergent composition comprises a chelator; further preferably said chelator is EDTA or citrate.

In one embodiment, the present invention relates to a detergent composition comprising an endoglucanase/xanthan lyase variant of the invention, wherein said variant has a half-life improvement factor (HIF) 1.0; preferably said variant has a half-life improvement factor (HIF) >1.0, preferably at least 1.2, such as at least 1.5, e.g. at least 2.0, relative to a parent endoglucanase/xanthan lyase. A preferred way of calculating said half-life improvement factor (HIF) is described in Examples 3 and 7 herein. Accordingly, residual activity (RA) can be calculated using the following formula: RA (%)=(Abs(Stress)/Abs(Ref) x 100%, wherein Abs(Stress) is the absorbance at 405 nm of the sample in the stress microtiter plate (MTP) (e.g. incubated at 25° C. over-night or any other combination of temperature and time) after subtracting relevant background absorbance contributions, Abs(Ref) is the absorbance at 405 nm of the sample in the reference MTP (e.g. incubated at 4° C. over-night) after subtracting relevant background absorbance contributions, wherein half-lives for the degradation of each variant and parent endoglucanase/xanthan lyase (e.g. at 25° C.) are calculated using the following formula: T½ (variant or parent)=(Ln (0.5)/Ln (RA-variant/100))*time, wherein “RA” is the residual activity in percent and “Time” is the incubation time in hours for both the stress and reference MTP, wherein half-life-improvement factors (HIFs) are calculated using the following formula: HIF =T½ (variant)/T½ (wild-type), e.g. wherein T½ wt (or wild type) is the T½ of the mature parent endoglucanase/xanthan lyase with SEQ ID NO:2 or 6.

In one embodiment the present invention relates to a detergent composition comprising an endoglucanase/xanthan lyase variant of the invention, wherein a half-life improvement factor (HIF) is determined after incubation of said endoglucanase/xanthan lyase variant in a detergent composition at 25° C. or 30° C. for a time period from about 30 min to about 20 h.

Parent

The parent endoglucanase may be (a) a polypeptide having at least 60% sequence identity to the mature polypeptide of SEQ ID NO:2 or 6; (b) a polypeptide encoded by a polynucleotide that hybridizes under low stringency conditions with (i) the mature polypeptide coding sequence of SEQ ID NO:1 or 5, or (ii) the full-length complement of (i); or (c) a polypeptide encoded by a polynucleotide having at least 60% sequence identity to the mature polypeptide coding sequence of SEQ ID NO:1 or 5.

In an aspect, the parent has a sequence identity to the mature polypeptide of SEQ ID NO:2 or 6 of at least 60%, e.g. at least 65%, 70%, 75%, 80%, 85%, 90%, 91%, 92%, 93%, 94%, 95%, 96%, 97%, 98%, 99%, or 100%, which have endoglucanase/xanthan lyase activity. In one aspect, the amino acid sequence of the parent differs by up to 10 amino acids, e.g. 1, 2, 3, 4, 5, 6, 7, 8, 9, or 10, from the mature polypeptide of SEQ ID NO:2 or 6.

In another aspect, the parent endoglucanase comprises or consists of the amino acid sequence of SEQ ID NO:2. In another aspect, the parent endoglucanase comprises or consists of the mature polypeptide of SEQ ID NO:2. In another aspect, the parent endoglucanase is a fragment of the mature polypeptide of SEQ ID NO:2 containing at least 85%, 86%, 87%, 88%, 89%, 90%, 91%, 92%, 93%, 94% or 95% of the number of amino acids of SEQ ID NO:2. In another embodiment, the parent endoglucanase is an allelic variant of the mature polypeptide of SEQ ID NO:2.

In another aspect, the parent xanthan lyase comprises or consists of the amino acid sequence of SEQ ID NO:6. In another aspect, the parent xanthan lyase comprises or consists of the mature polypeptide of SEQ ID NO:6. In another aspect, the parent xanthan lyase is a fragment of the mature polypeptide of SEQ ID NO:6 containing at least 85%, 86%, 87%, 88%, 89%, 90%, 91%, 92%, 93%, 94% or 95% of the number of amino acids of SEQ ID NO:6. In another embodiment, the parent xanthan lyase is an allelic variant of the mature polypeptide of SEQ ID NO:6.

In another aspect, the parent is encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO:1 or 5, or (ii) the full-length complement of (i) (Sambrook et al., 1989, Molecular Cloning, A Laboratory Manual, 2d edition, Cold Spring Harbor, N.Y.).

The polynucleotide of SEQ ID NO:1 or 5 or a subsequence thereof, as well as the polypeptide of SEQ ID NO:2 or 6 or a fragment thereof, may be used to design nucleic acid probes to identify and clone DNA encoding a parent from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g. at least 25, at least 35, or at least 70 nucleotides in length. Preferably, the nucleic acid probe is at least 100 nucleotides in length, e.g. at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a parent. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO:1 or 5 or a subsequence thereof, the carrier material is used in a Southern blot.

For purposes of the present invention, hybridization indicates that the polynucleotide hybridizes to a labeled nucleic acid probe corresponding to (i) SEQ ID NO:1 or 5; (ii) the mature polypeptide coding sequence of SEQ ID NO:1 or 5; (iii) the full-length complement thereof; or (iv) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is the mature polypeptide coding sequence of SEQ ID NO:1 or 5. In another aspect, the nucleic acid probe is a polynucleotide that encodes the polypeptide of SEQ ID NO:2 or 6; the mature polypeptide thereof; or a fragment thereof. In another aspect, the nucleic acid probe is SEQ ID NO:1 or 5.

In another embodiment, the parent is encoded by a polynucleotide having a sequence identity to the mature polypeptide coding sequence of SEQ ID NO:1 or 5 of at least 60%, e.g. at least 65%, 70%, 75%, 80%, 85%, 90%, 91%, 92%, 93%, 94%, 95%, 96%, 97%, 98%, 99%, or 100%.

The polypeptide may be a hybrid polypeptide in which a region of one polypeptide is fused at the N-terminus or the C-terminus of a region of another polypeptide.

The parent may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent may be obtained from microorganisms of any genus. For purposes of the present invention, the term “obtained from” as used herein in connection with a given source shall mean that the parent encoded by a polynucleotide is produced by the source or by a strain in which the polynucleotide from the source has been inserted. In one aspect, the parent is secreted extracellularly.

The parent may be a bacterial enzyme. For example, the parent may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces enzyme, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma enzyme.

In one aspect, the parent is a Bacillus alkalophilus, Bacillus amyloliquefaciens, Bacillus brevis, Bacillus circulans, Bacillus clausfi, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis enzyme.

In another aspect, the parent is a Streptococcus equisimilis, Streptococcus pyogenes, Streptococcus uberis, or Streptococcus equi subsp. Zooepidemicus enzyme.

In another aspect, the parent is a Streptomyces achromogenes, Streptomyces avermitilis, Streptomyces coelicolor, Streptomyces griseus, or Streptomyces lividans enzyme.

The parent may be a fungal enzyme. For example, the parent may be a yeast enzyme such as a Candida, Kluyveromyces, Pichia, Saccharomyces, Schizosaccharomyces, or Yarrowia enzyme; or a filamentous fungal enzyme such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Dipodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria enzyme.

In another aspect, the parent is a Saccharomyces carlsbergensis, Saccharomyces cerevisiae, Saccharomyces diastaticus, Saccharomyces douglasfi, Saccharomyces kluyveri, Saccharomyces norbensis, or Saccharomyces oviformis enzyme.

In another aspect, the parent is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, lrpex lacteus, Mucor miehei, Myceliophthora thermophila, Neurospora crassa, Penicillium funiculosum, Penicillium purpurogenum, Phanerochaete chtysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride enzyme.

In another aspect, the parent is a Paenibacillus sp. xanthan lyase, e.g. the xanthan lyase of SEQ ID NO:6.

In another aspect, the parent is a Paenibacillus sp. Endoglucanase of the GH9 family, e.g. the endoglucanase of SEQ ID NO:2.

It will be understood that for the aforementioned species, the invention encompasses both the perfect and imperfect states, and other taxonomic equivalents, e.g. anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche SammLung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The parent may be identified and obtained from other sources including microorganisms isolated from nature (e.g. soil, composts, water, etc.) or DNA samples obtained directly from natural materials (e.g. soil, composts, water, etc.) using the above-mentioned probes. Techniques for isolating microorganisms and DNA directly from natural habitats are well known in the art. A polynucleotide encoding a parent may then be obtained by similarly screening a genomic DNA or cDNA library of another microorganism or mixed DNA sample. Once a polynucleotide encoding a parent has been detected with the probe(s), the polynucleotide can be isolated or cloned by utilizing techniques that are known to those of ordinary skill in the art (see, e.g. Sambrook et al., 1989, supra).

### Embodiments

In one embodiment, the present invention relates to a detergent composition comprising at least one (e.g. 1, 2, 3, 4, 5, 6, 7, 8, 9 or 10) endoglucanase variant as described herein and at least one (e.g. 1, 2, 3, 4, 5, 6, 7, 8, 9 or 10) xanthan lyase variant as described herein.

In one embodiment, the detergent composition of the invention further comprises one or more additional enzymes selected from the group consisting of: proteases, amylases, lichenases, lipases, cutinases, cellulases, endoglucanases, xyloglucanases, pectinases, pectin lyases, xanthanases, peroxidases, haloperoxygenases, catalases and mannanases, or any mixture thereof.

The detergent compositions of the invention may further comprise one or more detergent components. In some embodiments said detergent component may be a chelator, such as EDTA or citrate.

In one embodiment, the detergent composition further comprises one or more detergent components, wherein said detergent composition is in form of a bar, a homogenous tablet, a tablet having two or more layers, a pouch having one or more compartments, a regular or compact powder, a granule, a paste, a gel, or a regular, compact or concentrated liquid.

In one embodiment, the present invention relates to use of a detergent composition of the invention, wherein said use is selected from the group of: use for degrading xanthan gum and use in a cleaning process, such as laundry or hard surface cleaning such as dish wash.

In one embodiment, the present invention relates to use of a detergent composition of the invention, wherein said composition has an enzyme detergency benefit.

In one embodiment the present invention relates to a method for degrading xanthan gum comprising: applying a detergent composition of the invention to a xanthan gum.

In one embodiment the present invention relates to a method for degrading xanthan gum comprising: applying a detergent composition of the invention to a xanthan gum, wherein said xanthan gum is on the surface of a textile or hard surface, such as dish wash.

Compositions

In one certain aspect, the variants according to the invention have improved stability in detergents compared to a parent enzyme or compared to an endoglucanase/xanthan lyase having the identical amino acid sequence of the variant, but not having an alteration (e.g. a substitution, deletion or insertion, preferably a substitution) at one or more of the specified positions or compared to the endoglucanase with the amino acid sequence set forth in SEQ ID NO:2 or the xanthan lyase with the amino aicd sequence as set forth in SEQ ID NO:6, wherein activity and/or stability in detergent is measured as disclosed in Examples 3 and 7 herein.

Besides enzymes the detergent compositions may comprise additional components. The choice of additional components is within the skill of the artisan and includes conventional ingredients, including the exemplary non-limiting components set forth below. The choice of components may include, for fabric care, the consideration of the type of fabric to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

The detergent composition may be suitable for the laundring of textiles such as e.g. fabrics, cloths or linen, or for cleaning hard surfaces such as e.g. floors, tables, or dish wash.

Detergent Compositions

In one embodiment, an endoglucanase variant as described herein may be added to a detergent composition in an amount corresponding to 0.0001-200 mg of enzyme protein, such as 0.0005-100 mg of enzyme protein, preferably 0.001-30 mg of enzyme protein, more preferably 0.005-8 mg of enzyme protein, even more preferably 0.01-2 mg of enzyme protein per litre of wash liquor.

In one embodiment, an xanthan lyase variant as described herein may be added to a detergent composition in an amount corresponding to 0.0001-200 mg of enzyme protein, such as 0.0005-100 mg of enzyme protein, preferably 0.001-30 mg of enzyme protein, more preferably 0.005-8 mg of enzyme protein, even more preferably 0.01-2 mg of enzyme protein per litre of wash liquor.

In some embodiments, each an endoglucanase variant as described herein and a xanthan lyase variant as described herein may be added to a detergent composition each in an amount corresponding to 0.0001-200 mg of enzyme protein, such as 0.0005-100 mg of enzyme protein, preferably 0.001-30 mg of enzyme protein, more preferably 0.005-8 mg of enzyme protein, even more preferably 0.01-2 mg of enzyme protein per litre of wash liquor.

A composition for use in automatic dishwash (ADW), for example, may include 0.0001-50%, such as 0.001-20%, such as 0.01-10%, such as 0.05-5% of each of the enzyme proteins by weight of the composition.

A composition for use in laundry granulation or a solid/granular laundry compositon in general, for example, may include 0.0001-50%, such as 0.001-20%, such as 0.01-10%, such as 0.05-5% of each of the enzyme proteins by weight of the composition.

A composition for use in laundry liquid, for example, may include 0.0001-10%, such as 0.001-7%, such as 0.1-5% of each of the enzyme proteins by weight of the composition.

The enzyme(s) of the detergent composition of the invention may be stabilized using conventional stabilizing agents, e.g. a polyol such as propylene glycol or glycerol, a sugar or sugar alcohol, lactic acid, boric acid, or a boric acid derivative, e.g. an aromatic borate ester, or a phenyl boronic acid derivative such as 4-formylphenyl boronic acid, and the composition may be formulated as described in, for example, WO92/19709 and WO92/19708.

In certain markets different wash conditions and, as such, different types of detergents are used. This is disclosed in e.g. EP1025240. For example, In Asia (Japan) a low detergent concentration system is used, while the United States uses a medium detergent concentration system, and Europe uses a high detergent concentration system.

A low detergent concentration system includes detergents where less than about 800 ppm of detergent components are present in the wash water. Japanese detergents are typically considered low detergent concentration system as they have approximately 667 ppm of detergent components present in the wash water.

A medium detergent concentration includes detergents where between about 800 ppm and about 2000 ppm of detergent components are present in the wash water. North American detergents are generally considered to be medium detergent concentration systems as they have approximately 975 ppm of detergent components present in the wash water.

A high detergent concentration system includes detergents where greater than about 2000 ppm of detergent components are present in the wash water. European detergents are generally considered to be high detergent concentration systems as they have approximately 4500-5000 ppm of detergent components in the wash water.

Latin American detergents are generally high suds phosphate builder detergents and the range of detergents used in Latin America can fall in both the medium and high detergent concentrations as they range from 1500-6000 ppm of detergent components in the wash water. Such detergent compositions are all embodiments of the invention.

A polypeptide of the present invention may also be incorporated in the detergent formulations disclosed in WO97/07202, which is hereby incorporated by reference.

Surfactants

The detergent composition may comprise one or more surfactants, which may be anionic and/or cationic and/or non-ionic and/or semi-polar and/or zwitterionic, or a mixture thereof. In preferred embodiments, the detergent compositions of the invention comprise at least one surfactant. In a particular embodiment, the detergent composition includes a mixture of one or more nonionic surfactants and one or more anionic surfactants. The surfactant(s) is typically present at a level of from about 0.1-60% by weight, such as about 1-40%, or about 3-20%, or about 3-10%. The surfactant(s) is chosen based on the desired cleaning application, and includes any conventional surfactant(s) known in the art. Any surfactant known in the art for use in detergents may be utilized.

When included therein the detergent will usually comprise from about 1-40% by weight, such as from about 5-30%, including from about 5-15%, or from about 20-25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein the detergent will usually comprise from about 0-10% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein the detergent will usually comprise from about 0.2-40% by weight of a non-ionic surfactant, for example from about 0.5-30%, in particular from about 1-20%, from about 3-10%, such as from about 3-5%, or from about 8-12%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein the detergent will usually comprise from about 0-10% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

When included therein the detergent will usually comprise from about 0-10% by weight of a zwitterionic surfactant. Non-limiting examples of zwitterionic surfactants include betaine, alkyldimethylbetaine, sulfobetaine, and combinations thereof.

Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however, the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may comprise 0-5% by weight, such as about 0.5-5%, or about 3-5%, of a hydrotrope. Any hydrotrope known in the art for use in detergents may be utilized. Non-limiting examples of hydrotropes include sodium benzene sulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

Builders and Co-Builders

The detergent composition may comprise about 0-65% by weight, such as about 5-45% of a detergent builder or co-builder, or a mixture thereof. In a dish wash deteregent, the level of builder is typically 40-65%, particularly 50-65%. The builder and/or co-builder may particularly be a chelating agent that forms water-soluble complexes with Ca and Mg. Any builder and/or co-builder known in the art for use in laundry detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g. SKS-6 from Hoechst), ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

The detergent composition may also comprise 0-20% by weight, such as about 5-10%, of a detergent co-builder, or a mixture thereof. The detergent composition may include include a co-builder alone, or in combination with a builder, for example a zeolite builder. Non-limiting examples of co-builders include homopolymers of polyacrylates or copolymers thereof, such as poly(acrylic acid)

(PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,M-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra-(methylenephosphonic acid) (EDTMPA), diethylenetriaminepentakis(methylenephosphonic acid) (DTPMPA or DTMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N, N-diacetic acid (α-ALDA), serine-N, N-diacetic acid (SEDA), isoserine-N, N-diacetic acid (ISDA), phenylalanine-N, N-diacetic acid (PHDA), anthranilic acid-N, N-diacetic acid (ANDA), sulfanilic acid-N, N-diacetic acid (SLDA), taurine-N, N-diacetic acid (TUDA) and sulfomethyl-N, N-diacetic acid (SMDA), N-(2-hydroxyethyl)ethylidenediamine-N,N;N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g. WO09/102854, U.S. Pat. No. 5,977,053

Bleaching Systems: The detergent may comprise 0-50% by weight, such as about 0.1-25%, of a bleaching system. Any bleaching system known in the art for use in laundry detergents may be utilized. Suitable bleaching system components include bleaching catalysts, photobleaches, bleach activators, sources of hydrogen peroxide such as sodium percarbonate and sodium perborates, preformed peracids and mixtures thereof. Suitable preformed peracids include, but are not limited to, peroxycarboxylic acids and salts, percarbonic acids and salts, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with peroxygen bleach like hydrogen peroxide to form a peracid. The peracid thus formed constitutes the activated bleach. Suitable bleach activators to be used herein include those belonging to the class of esters amides, imides or anhydrides. Suitable examples are tetracetylethylene diamine (TAED), sodium 4-[(3,5,5-trimethylhexanoyl)oxy]benzene sulfonate (ISONOBS), diperoxy dodecanoic acid, 4-(dodecanoyloxy)benzenesulfonate (LOBS), 4-(decanoyloxy)benzenesulfonate, 4-(decanoyloxy)benzoate (DOBS), 4-(nonanoyloxy)-benzenesulfonate (NOBS), and/or those disclosed in WO98/17767. A particular family of bleach activators of interest was disclosed in EP624154 and particulary preferred in that family is acetyl triethyl citrate (ATC). ATC or a short chain triglyceride like triacetin has the advantage that it is environmental friendly as it eventually degrades into citric acid and alcohol. Furthermore acetyl triethyl citrate and triacetin has a good hydrolytical stability in the product upon storage and it is an efficient bleach activator. Finally ATC provides a good building capacity to the laundry additive. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

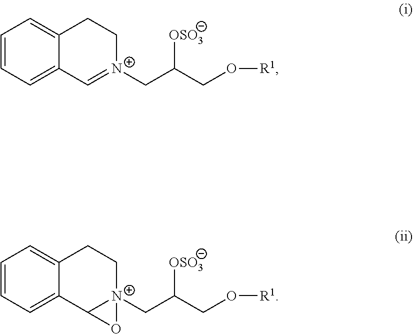

(iii) and mixtures thereof; wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl. Other exemplary bleaching systems are described, e.g. in WO02007/087258, WO2007/087244, WO2007/087259 and WO2007/087242. Suitable photobleaches may for example be sulfonated zinc phthalocyanine.

Polymers: The detergent may comprise 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g. WO 2006/130575. Salts of the above-mentioned polymers are also contemplated.

Fabric hueing agents:The detergent compositions of the present invention may also comprise fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003-0.2 wt %, from about 0.00008-0.05 wt %, or even from about 0.0001-0.04 wt % fabric hueing agent. The composition may comprise from 0.0001-0.2 wt % fabric hueing agent, this may be especially preferred when the composition is in the form of a unit dose pouch. Suitable hueing agents are also disclosed in, e.g. WO2007/087257 and WO2007/087243.

Additional Enzymes

The detergent additive as well as the detergent composition may comprise one or more [additional] enzymes such as a protease, lipase, cutinase, an amylase, lichenase, carbohydrase, cellulase, pectinase, mannanase, arabinase, galactanase, xylanase, oxidase, e.g. a laccase, and/or peroxidase.

In general, the properties of the selected enzyme(s) should be compatible with the selected detergent, (i.e. pH-optimum, compatibility with other enzymatic and non-enzymatic ingredients, etc.), and the enzyme(s) should be present in effective amounts.

Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g. the fungal cellulases produced from Humicola insolens, Myceliophthora thermophila and Fusarium oxysporum disclosed in U.S. Pat. Nos. 4,435,307, 5,648,263, 5,691,178, 5,776,757 and WO89/09259.

Especially suitable cellulases are the alkaline or neutral cellulases having color care benefits. Examples of such cellulases are cellulases described in EP0495257, EP0531372, WO96/11262, WO96/29397, WO98/08940. Other examples are cellulase variants such as those described in WO94/07998, EP0531315, U.S. Pat. Nos. 5,457,046, 5,686,593, 5,763,254, WO95/24471, WO98/12307 and PCT/DK98/00299.

Example of cellulases exhibiting endo-beta-1,4-glucanase activity (EC 3.2.1.4) are those having described in WO02/099091.

Other examples of cellulases include the family 45 cellulases described in WO96/29397, and especially variants thereof having substitution, insertion and/or deletion at one or more of the positions corresponding to the following positions in SEQ ID NO:8 of WO02/099091: 2, 4, 7, 8, 10, 13, 15, 19, 20, 21, 25, 26, 29, 32, 33, 34, 35, 37, 40, 42, 42a, 43, 44, 48, 53, 54, 55, 58, 59, 63, 64, 65, 66, 67, 70, 72, 76, 79, 80, 82, 84, 86, 88, 90, 91, 93, 95, 95d, 95h, 95j, 97, 100, 101, 102, 103, 113, 114, 117, 119, 121, 133, 136, 137, 138, 139, 140a, 141, 143a, 145, 146, 147, 150e, 150j, 151, 152, 153, 154, 155, 156, 157, 158, 159, 160c, 160e, 160k, 161, 162, 164, 165, 168, 170, 171, 172, 173, 175, 176, 178, 181, 183, 184, 185, 186, 188, 191, 192, 195, 196, 200, and/or 20, preferably selected among P19A, G20K, Q44K, N48E, Q119H or Q146 R.

Commercially available cellulases include Celluzyme™, and Carezyme™ (Novozymes NS), Clazinase™, and Puradax HA™ (Genencor International Inc.), and KAC-500(B)™ (Kao Corporation).

Proteases

The additional enzyme may be another protease or protease variant. The protease may be of animal, vegetable or microbial origin, including chemically or genetically modified mutants. Microbial origin is preferred. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4, M5, M7 or M8.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family. In one aspect of the invention the protease may be a subtilase, such as a subtilisin or a variant hereof. Further the subtilases (and the serine proteases) are characterised by having two active site amino acid residues apart from the serine, namely a histidine and an aspartic acid residue.

Examples of subtilisins are those derived from Bacillus such as subtilisin lentus, Bacillus lentus, subtilisin Novo, subtilisin Carlsberg, Bacillus licheniformis, subtilisin BPN′, subtilisin 309, subtilisin 147 and subtilisin 168 described in WO89/06279 and protease PD138 (WO93/18140). Additional serine protease examples are described in WO98/020115, WO01/44452, WO01/58275, WO01/58276, WO03/006602 and WO04/099401. An example of a subtilase variants may be those having mutations in any of the positions: 3, 4, 9, 15, 27, 36, 68, 76, 87, 95, 96, 97, 98, 99, 100, 101, 102, 103, 104, 106, 118, 120, 123, 128, 129, 130, 160, 167, 170, 194, 195, 199, 205, 217, 218, 222, 232, 235, 236, 245, 248, 252 and 274 using the BPN' numbering. More preferred the subtilase variants may comprise the mutations: S3T, V4I, S9R, A15T, K27R, *36D, V68A, N76D, N87S,R, *97E, A98S, S99G,D,A, S99AD, S101G,M,R S103A, V104I, Y,N, S106A, G118V,R, H120D,N, N123S, S128L, P129Q, S130A, G160D, Y167A, R170S, A194P, G195E, V199M, V205I, L217D, N218D, M222S, A232V, K235L, Q236H, Q245R, N252K, T274A (using BPN′ numbering). A further preferred protease is the alkaline protease from Bacillus lentus DSM 5483, as described for example in WO95/23221, and variants thereof which are described in WO92/21760, WO95/23221, EP1921147 and EP1921148.

Examples of trypsin-like proteases are trypsin (e.g. of porcine or bovine origin) and the Fusarium protease described in WO89/06270 and WO94/25583. Examples of useful proteases are the variants described in WO92/19729, WO98/20115, WO98/20116, and WO98/34946, especially the variants with substitutions in one or more of the following positions: 27, 36, 57, 76, 87, 97, 101, 104, 120, 123, 167, 170, 194, 206, 218, 222, 224, 235, and 274.

Examples of metalloproteases are the neutral metalloprotease as described in WO 07/044993.

Preferred commercially available protease enzymes include Alcalase™, Coronase™, Duralase™, Durazym™, Esperase™, Everlase™, Kannase™, Liquanase™, Liquanase Ultra™, Ovozyme™, Polarzyme™, Primase™, Relase™, Savinase™ and Savinase Ultra™, (Novozymes NS), Axapem™ (Gist-Brocases N.V.), BLAP and BLAP X (Henkel AG & Co. KGaA), Excellase™, FN2™, FN3™, FN4™, Maxaca™, Maxapem™, Maxatase™, Properase™, Purafast™, Purafect™, Purafect OxP™, Purafect Prime™ and Puramax™ (Genencor int.).

Lipases and Cutinases

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Further examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include include Lipolase™, Lipex™; Lipolex™ and Lipoclean™ (Novozymes NS), Lumafast (originally from Genencor) and Lipomax (originally from Gist-Brocades).

Amylases

The amylase may be an alpha-amylase, a beta-amylase or a glucoamylase and may be of bacterial or fungal origin. Chemically modified or protein engineered mutants are included. Amylases include, for example, alpha-amylases obtained from Bacillus, e.g. a special strain of Bacillus licheniformis, described in more detail in GB1296839.

Examples of amylases are those having SEQ ID NO:3 in WO95/10603 or variants having 90% sequence identity to SEQ ID NO:3 thereof. Preferred variants are described in WO94/02597, WO94/18314, WO97/43424 and SEQ ID NO:4 of WO99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444 of SEQ ID NO:3 in WO95/10603.

Further amylases which can be used are amylases having SEQ ID NO:6 in WO02/010355 or variants thereof having 90% sequence identity to SEQ ID NO:6. Preferred variants of SEQ ID NO:6 are those having a deletion in positions 181 and 182 and a substitution in position 193.

Other amylase examples are hybrid alpha-amylase comprising residues 1-33 of the alpha-amylase derived from B. amyloliquefaciens shown in SEQ ID NO:6 of WO2006/066594 and residues 36-483 of the B. licheniformis alpha-amylase shown in SEQ ID NO:4 of WO2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO:6 of WO2006/066594 and residues 36-483 of SEQ ID NO:4 are those having the substitutions: M197T; H156Y+A181T+N190F+A209V+Q264S; or G48+T49+G107+H156+A181+N190+I201+A209+Q264.

Further amylase examples are amylases having SEQ ID NO:6 in WO99/019467 or variants thereof having 90% sequence identity to SEQ ID NO:6. Preferred variants of SEQ ID NO:6 are those having a substitution, a deletion or an insertion in one or more of the following positions: R181, G182, H183, G184, N195, I206, E212, E216 and K269. Particularly preferred amylases are those having deletion in positions G182 and H183 or positions H183 and G184.

Additional amylases are those having SEQ ID NO:1, SEQ ID NO:2 or SEQ ID NO:7 of WO96/023873 or variants thereof having 90% sequence identity to SEQ ID NO:1, SEQ ID NO:2 or SEQ ID NO:7. Preferred variants of SEQ ID NO:1, SEQ ID NO:2 or SEQ ID NO:7 are those having a substitution, a deletion or an insertion in one or more of the following positions: 140, 181, 182, 183, 184, 195, 206, 212, 243, 260, 269, 304 and 476. More preferred variants are those having a deletion in positions 182 and 183 or positions 183 and 184. Most preferred amylase variants of SEQ ID NO:1, SEQ ID NO:2 or SEQ ID NO:7 are those having a deletion in positions 183 and 184 and a substitution in positions 140, 195, 206, 243, 260, 304 and 476.

Other amylases which can be used are amylases having SEQ ID NO:2 of WO08/153815, SEQ ID NO:10 in WO01/66712 or variants thereof having 90% sequence identity to SEQ ID NO:2 of WO08/153815 or 90% sequence identity to SEQ ID NO:10 in WO 01/66712. Preferred variants of SEQ ID NO:10 in WO01/66712 are those having a substitution, a deletion or an insertion in one of more of the following positions: 176, 177, 178, 179, 190, 201, 207, 211 and 264.

Further amylases which can be used are amylases having SEQ ID NO:2 of WO09/061380 or variants thereof having 90% sequence identity to SEQ ID NO:2. Preferred variants of SEQ ID NO:2 are those having a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO:2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181. Most preferred amylase variants of SEQ ID NO:2 are those having the substitutions: N128C+K178L+T182G+Y305R+G475K; N128C+K178L+T182G+F202Y+Y305R+D319T+G475K; S125A+N128C+K178L+T182G+Y305R+G475K; or S125A+N128C+T1311+T1651+K178L+T182G+Y305R+G475K wherein the variant optionally further comprises a substitution at position 243 and/or a deletion at position 180 and/or position 181.

Other amylases are variants of SEQ ID NO:1 of WO2016/203064 having at least 75% sequence identity to SEQ ID NO:1 thereof. Preferred variants are variants comprising a modification in one or more positions corresponding to positions 1, 54, 56, 72, 109, 113, 116, 134, 140, 159, 167, 169, 172, 173, 174, 181, 182, 183, 184, 189, 194, 195, 206, 255, 260, 262, 265, 284, 289, 304, 305, 347, 391, 395, 439, 469, 444, 473, 476, or 477 of SEQ ID NO:1, wherein said alpha-amylase variant has a sequence identity of at least 75% but less than 100% to SEQ ID NO:1.

Other examples of amylases are the alpha-amylase having SEQ ID NO:12 in WO01/66712 or a variant having at least 90%, such as at least 95%, sequence identity to SEQ ID NO:12. Preferred amylase variants are those having a substitution, a deletion or an insertion in one of more of the following positions of SEQ ID NO:12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Commercially available amylases are Duramyl™, Termamyl™, Fungamyl™, Stainzyme™, Stainzyme Plus™, Natalase™ and BAN™ (Novozymes NS), Rapidase™ and Purastar™ (from Genencor International Inc.).

Peroxidases/Oxidases: Suitable peroxidases/oxidases include those of plant, bacterial or fungal origin. Chemically modified or protein engineered mutants are included. Examples of useful peroxidases include peroxidases from Coprinus, e.g. from C. cinereus, and variants thereof as those described in WO93/24618, WO95/10602, and WO98/15257. Commercially available peroxidases include Guardzyme™ (Novozymes NS).

The detergent enzyme(s) may be included in a detergent composition by adding separate additives containing one or more enzymes, or by adding a combined additive comprising all of these enzymes. A detergent additive of the invention, i.e. a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000;

ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP238216.

Adjunct materials: Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants: The detergent compositions of the present invention can also contain dispersants. In particular, powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents: The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001-10%, from about 0.01-5% or even from about 0.1-3% by weight of the composition.

Fluorescent whitening agent: The detergent compositions of the present invention will preferably also contain additional components that may tint articles being cleaned, such as fluorescent whitening agent or optical brighteners. Where present the brightener is preferably at a level of about 0,01-0,5%. Any fluorescent whitening agent suitable for use in a laundry detergent composition may be used in the composition of the present invention. The most commonly used fluorescent whitening agents are those belonging to the classes of diaminostilbene-sulphonic acid derivatives, diarylpyrazoline derivatives and bisphenyl-distyryl derivatives. Examples of the diaminostilbene-sulphonic acid derivative type of fluorescent whitening agents include the sodium salts of: 4,4′-bis-(2-diethanolamino-4-anilino-s-triazin-6-ylamino) stilbene-2,2′-disulphonate; 4,4′-bis-(2,4-dianilino-s-triazin-6-ylamino) stilbene-2.2′-disulphonate; 4,4′-bis-(2-anilino-4(N-methyl-N-2-hydroxy-ethylamino)-s-triazin-6-ylamino) stilbene-2,2′-disulphonate, 4,4′-bis-(4-phenyl-2,1,3-triazol-2-yl)stilbene-2,2′-disulphonate; 4,4′-bis-(2-anilino-4(1-methyl-2-hydroxy-ethylamino)-s-triazin-6-ylamino) stilbene-2,2′-disulphonate and 2-(stilbyl-4″-naptho-1.,2′:4,5)-1,2,3-trizole-2″-sulphonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4 anilino-s-triazin-6-ylamino) stilbene disulphonate. Tinopal CBS is the disodium salt of 2,2′-bis-(phenyl-styryl) disulphonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt.% to upper levels of 0.5 or even 0.75 wt %.

Soil release polymers: The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers is amphiphilic alkoxylated grease cleaning polymers comprising a core structure and a plurality of alkoxylate groups attached to that core structure. The core structure may comprise a polyalkylenimine structure or a polyalkanolamine structure as described in detail in WO2009/087523 (hereby incorporated by reference). Furthermore random graft co-polymers are suitable soil release polymers Suitable graft co-polymers are described in more detail in WO2007/138054, WO 2006/108856 and WO2006/113314 (hereby incorporated by reference). Other soil release polymers are substituted polysaccharide structures especially substituted cellulosic structures such as modified cellulose deriviatives such as those described in EP1867808 or WO2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-redeposition agents: The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

Formulation of Detergent Products

The detergent composition may be in any convenient form, e.g. a bar, a homogenous tablet, a tablet having two or more layers, a pouch having one or more compartments, a regular or compact powder, a granule, a paste, a gel, or a regular, compact or concentrated liquid. There are a number of detergent formulation forms such as layers (same or different phases), pouches, as well as forms for machine dosing unit.

Pouches can be configured as single or multicompartments. It can be of any form, shape and material which is suitable for hold the composition, e.g. without allowing the release of the composition from the pouch prior to water contact. The pouch is made from water soluble film which encloses an inner volume. Said inner volume can be devided into compartments of the pouch. Preferred films are polymeric materials preferably polymers which are formed into a film or sheet. Preferred polymers, copolymers or derivates therof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxyprpyl methyl cellulose (HPMC). Preferably the level of polymer in the film for example PVA is at least about 60%. Preferred average molecular weight will typically be about 20,000 to about 150,000. Films can also be of blend compositions comprising hydrolytically degradable and water soluble polymer blends such as polyactide and polyvinyl alcohol (known under the Trade reference M8630 as sold by Chris Craft In. Prod. Of Gary, Ind., US) plus plasticisers like glycerol, ethylene glycerol, Propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids. Ref: (US2009/0011970A1).

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

Laundry Soap Bars

The enzymes of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, an enzyme, optionally one or more additional enzymes, a protease inhibitor, and a salt of a monovalent cation and an organic anion may be prepared and and the mixture is then plodded. The enzyme and optional additional enzymes may be added at the same time as the protease inhibitor for example in liquid form. Besides the mixing step and the plodding step, the process may further comprise the steps of milling, extruding, cutting, stamping, cooling and/or wrapping.

Method of Producing the Composition

The present invention also relates to methods of producing the composition. The method may be relevant for the (storage) stability of the detergent composition: e.g. Soap bar premix method WO2009155557.

Uses

The present invention is also directed to methods for using the detergent compositions thereof. The present invention may be used for example in any detergent application which requries the degradation of xanthan gum.

Use to Degrade Xanthan Gum

Xanthan gum has been used as an ingredient in many consumer products including foods and cosmetics and has found use in the oil industry. Therefore, the degradation of xanthan gum can result in improved cleaning processes, such as the easier removal of stains containing gums, such as xanthan gum. Thus, the present invention is directed to the use of detergent compositions comprising GH9 endoglucanases (e.g. variants described herein) of the invention to degrade xanthan gum. The present invention is also directed to the use of xanthan lyases in the compositions of the invention to degrade xanthan gum. An embodiment is the use of a detergent composition comprising GH9 endoglucanases as described herein (e.g. variants) together with xanthan lyases to degrade xanthan gum. Degradation of xanthan gum can preferably be measured using the viscosity reduction assay (e.g. ViPr assay) or alternatively as describred in Examples 3 and 7 herein.

GH9 endoglucanase activity may alternatively be measured by assessment of reducing ends on xanthan gum pre-treated with xanthan lyase using the colorimetric assay developed by Lever (1972), Anal. Biochem. 47: 273-279, 1972. A preferred embodiment is the use of 0.1% xanthan gum pre-treated with xanthan lyase. Degradation of xanthan gum pre-treated with xanthan lyase may be determined by calculating difference between blank and sample, wherein a difference of more than 0.5 mAU, preferably more than 0.6 mAU, more preferably more than 0.7 mAU or even more preferably more than 0.8 mAU, shows degradation of xanthan gum pre-treated with xanthan lyase.

Xanthan lyase activity may alternatively be measured by assessment of reducing ends liberated from xanthan gum using the colorimetric assay developed by Lever (1972), Anal. Biochem. 47: 273-279, 1972. A preferred embodiment is the use of 0.1% xanthan gum. Degradation of xanthan gum may be determined by calculating difference between blank and sample wherein a difference of more than 0.1 mAU, preferably more than 0.15 mAU, more preferably more than 0.2 mAU or even more preferably more than 0.25 mAU shows degradation of xanthan gum.

GH9 endoglucanase and xanthan lyase activity may alternatively be measured by assessment of reducing ends liberated from xanthan gum using the colorimetric assay developed by Lever (1972), Anal. Biochem. 47: 273-279, 1972. A preferred embodiment is the use of 0.1% xanthan gum. Degradation of xanthan gum may be determined by calculating difference between blank and sample wherein a difference of more than 0.4 mAU, preferably more than 0.5 mAU, more preferably more than 0.6 mAU or even more preferably more than 0.8 mAU shows degradation of xanthan gum.

The invention also relates to methods for degrading xanthan gum comprising applying a detergent composition comprising one or more GH9 endoglucanases described herein (e.g. variants) and one or more xanthan lyases described herein (e.g. variants) to xanthan gum.

Use in Detergents

The present invention inter alia relates to the use of detergent compositions comprising GH9 endoglucanases and xanthan lyases as described herein (e.g. variants) in cleaning processes such as the laundering of textiles and fabrics (e.g. household laundry washing and industrial laundry washing), as well as household and industrial hard surface cleaning, such as dish wash. For this, the GH9 endoglucanases and xanthan lyases (e.g. variants) may be added to a detergent composition comprising of one or more detergent components.

The polypeptides described herein (e.g. variants) may be added to and thus become a component of a detergent composition. The detergent composition may be formulated, for example, as a hand or machine laundry detergent composition for both household and industrial laundry cleaning, including a laundry additive composition suitable for pre-treatment of stained fabrics and a rinse added fabric softener composition, or be formulated as a detergent composition for use in general household or industrial hard surface cleaning operations, or be formulated for hand or machine (both household and industrial) dishwashing operations. In a specific aspect, the present invention relates to a detergent additive comprising a polypeptide as described herein.

The invention also relates to methods for degrading xanthan gum on the surface of a textile or hard surface, such as dish wash, comprising applying a detergent composition as described herein to xanthan gum.

It has been contemplated that the use of a GH9 endoglucanase and xanthan lyase as described herein (e.g. a variant as described herein) alone gives an enzyme detergency benefit, preferably an enzyme detergency benefit on xanthan gum.

In some aspects, the invention relates to the use of a detergent composition comprising one or more detergent components and an isolated GH9 endoglucanase described herein (e.g. a variant) together with an isolated xanthan lyase described herein (e.g. variant).

The invention is further defined in the following paragraphs:

1. A detergent composition comprising

(A) an endoglucanase variant, comprising an alteration (e.g. a substitution, deletion or insertion, preferably a substitution) at one or more positions in a region selected from the group consisting of:

i) region 1 corresponding to amino acids 95 to 105 of SEQ ID NO:2, e.g. said alteration at one or more positions selected from the group consisting of positions: 95, 96, 97, 98, 99, 100, 101, 102, 103, 104, 105, wherein said positions correspond to amino acid positions of SEQ ID NO:2 (e.g. using the numbering of SEQ ID NO:2),

ii) region 2 corresponding to amino acids 115 to 138 of SEQ ID NO:2, e.g. said alteration at one or more positions selected from the group consisting of positions: 115, 116, 117, 118, 119, 120, 121, 122, 123, 124, 125, 126, 127, 128, 129, 130, 131, 132, 133, 134, 135, 136, 137, 138, wherein said positions correspond to amino acid positions of SEQ ID NO:2 (e.g. using the numbering of SEQ ID NO:2),

iii) region 3 corresponding to amino acids 210 to 251 of SEQ ID NO:2, e.g. said alteration at one or more positions selected from the group consisting of positions: 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, wherein said positions correspond to amino acid positions of SEQ ID NO:2 (e.g. using the numbering of SEQ ID NO:2),

iv) region 4 corresponding to amino acids 267 to 301 of SEQ ID NO:2, e.g. said alteration at one or more positions selected from the group consisting of positions: 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, wherein said positions correspond to amino acid positions of SEQ ID NO:2 (e.g. using the numbering of SEQ ID NO:2),

v) region 5 corresponding to amino acids 339 to 361 of SEQ ID NO:2, e.g. said alteration at one or more positions selected from the group consisting of positions: 339, 340, 341, 342, 343, 344, 345, 346, 347, 348, 349, 350, 351, 352, 353, 354, 355, 356, 357, 358, 359, 360, 361, wherein said positions correspond to amino acid positions of SEQ ID NO:2 (e.g. using the numbering of SEQ ID NO:2),

vi) region 6 corresponding to amino acids 547 to 595 of SEQ ID NO:2, e.g. said alteration at one or more positions selected from the group consisting of positions: 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, wherein said positions correspond to amino acid positions of SEQ ID NO:2 (e.g. using the numbering of SEQ ID NO:2),

vii) region 7 corresponding to amino acids 612 to 660 of SEQ ID NO:2, e.g. said alteration at one or more positions selected from the group consisting of positions: 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, wherein said positions correspond to amino acid positions of SEQ ID NO:2 (e.g. using the numbering of SEQ ID NO:2),

viii) region 8 corresponding to amino acids 806 to 828 of SEQ ID NO:2, e.g. said alteration at one or more positions selected from the group consisting of positions: 806, 807, 808, 809, 810, 811, 812, 813, 814, 815, 816, 817, 818, 819, 820, 821, 822, 823, 824, 825, 826, 827, 828, wherein said positions correspond to amino acid positions of SEQ ID NO:2 (e.g. using the numbering of SEQ ID NO:2), and

ix) region 9 corresponding to amino acids 839 to 1042 of SEQ ID NO:2, e.g. said alteration at one or more positions selected from the group consisting of positions: 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, wherein said positions correspond to amino acid positions of SEQ ID NO:2 (e.g. using the numbering of SEQ ID NO:2),

wherein said variant has at least 60%, e.g. at least 65%, 70%, 75%, 80%, 85%, 90%, 95%, 96%, 97%, 98%, or 99%, and less than 100% sequence identity to SEQ ID NO:2; preferably said endoglucanase variant has activity on xanthan gum pretreated with xanthan lyase, further preferably said activity is a xanthan gum degrading activity; and

(B) a xanthan lyase variant, comprising an alteration (e.g. a substitution, deletion or insertion, preferably a substitution) at one or more positions in a region selected from the group consisting of: (i) region 1 corresponding to amino acids 154 to 176 of SEQ ID NO:6; (ii) region 2 corresponding to amino acids 614 to 658 of SEQ ID NO:6; (iii) region 3 corresponding to amino acids 731 to 803 of SEQ ID NO:6; (iv) region 4 corresponding to amino acids 807 to 846 of SEQ ID NO:6; (v) region 5 corresponding to amino acids 872 to 885 of SEQ ID NO:6; (vi) region 6 corresponding to amino acids 903 to 1004 of SEQ ID NO:6; (vii) region 7 corresponding to amino acids 1 to 153 of SEQ ID NO:6; (viii) region 8 corresponding to amino acids 177 to 613 of SEQ ID NO:6; (ix) region 9 corresponding to amino acids 659 to 730 of SEQ ID NO:6; (x) region 10 corresponding to amino acids 804 to 806 of SEQ ID NO:6; (xi) region 11 corresponding to amino acids 847 to 871 of SEQ ID NO:6; (xii) region 12 corresponding to amino acids 886 to 902 of SEQ ID NO:6; (xiii) region 13 corresponding to amino acids 1005 to 1037 of SEQ ID NO:6; wherein said variant has at least 60%, e.g. at least 65%, 70%, 75%, 80%, 85%, 90%, 95%, 96%, 97%, 98%, or 99%, and less than 100% sequence identity to SEQ ID NO:6; preferably said xanthan lyase variant has activity on xanthan gum, further preferably said activity is a xanthan gum degrading activity.

2. The detergent composition comprising an endoglucanase variant of paragraph 1, which is a variant of a parent endoglucanase selected from the group consisting of: (a) a polypeptide having at least 60% sequence identity to the mature polypeptide of SEQ ID NO:2; (b) a polypeptide encoded by a polynucleotide that hybridizes under low stringency conditions with (i) the mature polypeptide coding sequence of SEQ ID NO:1, or (ii) the full-length complement of (i); (c) a polypeptide encoded by a polynucleotide having at least 60% identity to the mature polypeptide coding sequence of SEQ ID NO:1; and (d) a fragment of the mature polypeptide of SEQ ID NO:2, which has endoglucanase activity.

3. The detergent composition comprising an endoglucanase variant of paragraph 2, wherein the parent endoglucanase having at least 60%, e.g. at least 65%, 70%, 75%, 80%, 85%, 90%, 95%, 96%, 97%, 98%, 99% or 100% sequence identity to the mature polypeptide of SEQ ID NO:2.

4. The detergent composition comprising an endoglucanase variant of any of paragraphs 2-3, wherein the parent endoglucanase is encoded by a polynucleotide that hybridizes under low stringency conditions, medium stringency conditions, medium-high stringency conditions, high stringency conditions, or very high stringency conditions with (i) the mature polypeptide coding sequence of SEQ ID NO:1 or (ii) the full-length complement of (i).

5. The detergent composition comprising an endoglucanase variant of any of paragraphs 2-4, wherein the parent endoglucanase is encoded by a polynucleotide having at least 60%, e.g. at least 65%, 70%, 75%, 80%, 85%, 90%, 95%, 96%, 97%, 98%, 99%, or 100% sequence identity to the mature polypeptide coding sequence of SEQ ID NO:1.

6. The detergent composition comprising an endoglucanase variant of any of paragraphs 2-5, wherein the parent endoglucanase comprises or consists of the mature polypeptide of SEQ ID NO:2.

7. The detergent composition comprising an endoglucanase variant of any of paragraphs 2-6, wherein the parent endoglucanase is a fragment of the mature polypeptide of SEQ ID NO:2, wherein the fragment has endoglucanase activity.

8. The detergent composition comprising an endoglucanase variant of any of paragraphs 2-7, which has at least 60%, e.g. at least 65%, 70%, 75%, 80%, 85%, 90%, 95%, 96%, 97%, 98%, or 99%, but less than 100%, sequence identity to the amino acid sequence of the parent endoglucanase.

9. The detergent composition comprising an endoglucanase variant of any of paragraphs 1-8, wherein said region selected from the group consisting of regions 1-9 is a chelator-induced instability region;

preferably said chelator-induced instability region (e.g. of SEQ ID NO:2 or another parent endoglucanase) has one or more of the following features: (i) in the presence of a chelator it is less conformationally stable than one or more or all of its adjacent regions; and/or (ii) in the presence of a chelator it is more exposed to said chelator than one or more or all of its adjacent regions; and/or (iii) in the presence of a chelator it is more accessible to said chelator than one or more or all of its adjacent regions; and/or (iv) in the presence of a chelator it is more conformationally dynamic than one or more or all of its adjacent regions; and/or (v) in the presence of a chelator it is more receptive to deuterium incorporation than one or more or all of its adjacent regions;

further preferably said adjacent region is selected from the group consisting of: (vi) region 10 corresponding to amino acids 1 to 94 of SEQ ID NO:2; (vii) region 11 corresponding to amino acids 106 to 114 of SEQ ID NO:2; (viii) region 12 corresponding to amino acids 139 to 209 of SEQ ID NO:2; (ix) region 13 corresponding to amino acids 252 to 266 of SEQ ID NO:2; (x) region 14 corresponding to amino acids 302 to 338 of SEQ ID NO:2; (xi) region 15 corresponding to amino acids 362 to 546 of SEQ ID NO:2; (xii) region 16 corresponding to amino acids 596 to 611 of SEQ ID NO:2; (xiii) region 17 corresponding to amino acids 661 to 805 of SEQ ID NO:2; (xiv) region 18 corresponding to amino acids 829 to 838 of SEQ ID NO:2; and(xv) region 19 corresponding to amino acids 1043 to 1055 of SEQ ID NO:2;

further most preferably said chelator is EDTA or citrate.

10. The detergent composition comprising an endoglucanase variant of any one of paragraphs 1-9, wherein said variant further comprises an alteration in at least one adjacent region, said adjacent region is selected from the group consisting of: (i′) region 10 corresponding to amino acids 1 to 94 of SEQ ID NO:2; (ii′) region 11 corresponding to amino acids 106 to 114 of SEQ ID NO:2; (iii′) region 12 corresponding to amino acids 139 to 209 of SEQ ID NO:2; (iv′) region 13 corresponding to amino acids 252 to 266 of SEQ ID NO:2; (v′) region 14 corresponding to amino acids 302 to 338 of SEQ ID NO:2; (vi′) region 15 corresponding to amino acids 362 to 546 of SEQ ID NO:2; (vii′) region 16 corresponding to amino acids 596 to 611 of SEQ ID NO:2; (viii′) region 17 corresponding to amino acids 661 to 805 of SEQ ID NO:2; (ix′) region 18 corresponding to amino acids 829 to 838 of SEQ ID NO:2; and (x′) region 19 corresponding to amino acids 1043 to 1055 of SEQ ID NO:2.

11. The detergent composition comprising an endoglucanase variant of any of paragraphs 1-10, wherein in an aqueous solution comprising a detergent component said region (e.g. of SEQ ID NO:2 or another parent endoglucanase) selected from the group consisting of regions 1-9 is less conformationally stable than one or more or all of its adjacent regions;

preferably said adjacent region is selected from the group consisting of: (i) region 10 corresponding to amino acids 1 to 94 of SEQ ID NO:2; (ii) region 11 corresponding to amino acids 106 to 114 of SEQ ID NO:2; (iii) region 12 corresponding to amino acids 139 to 209 of SEQ ID NO:2; (iv) region 13 corresponding to amino acids 252 to 266 of SEQ ID NO:2; (v) region 14 corresponding to amino acids 302 to 338 of SEQ ID NO:2; (vi) region 15 corresponding to amino acids 362 to 546 of SEQ ID NO:2; (vii) region 16 corresponding to amino acids 596 to 611 of SEQ ID NO:2; (viii) region 17 corresponding to amino acids 661 to 805 of SEQ ID NO:2; (ix) region 18 corresponding to amino acids 829 to 838 of SEQ ID NO:2; and (x) region 19 corresponding to amino acids 1043 to 1055 of SEQ ID NO:2;

further preferably said detergent component comprises a chelator; further most preferably said chelator is EDTA or citrate.

12. The detergent composition comprising an endoglucanase variant of any of paragraphs 1-11, wherein in an aqueous solution comprising a detergent component said region (e.g. of SEQ ID NO:2 or another parent endoglucanase) selected from the group consisting of regions 1-9 is more exposed to said detergent component than one or more or all of its adjacent regions;

preferably said adjacent region is selected from the group consisting of: (i) region 10 corresponding to amino acids 1 to 94 of SEQ ID NO:2; (ii) region 11 corresponding to amino acids 106 to 114 of SEQ ID NO:2; (iii) region 12 corresponding to amino acids 139 to 209 of SEQ ID NO:2; (iv) region 13 corresponding to amino acids 252 to 266 of SEQ ID NO:2; (v) region 14 corresponding to amino acids 302 to 338 of SEQ ID NO:2; (vi) region 15 corresponding to amino acids 362 to 546 of SEQ ID NO:2; (vii) region 16 corresponding to amino acids 596 to 611 of SEQ ID NO:2; (viii) region 17 corresponding to amino acids 661 to 805 of SEQ ID NO:2; (ix) region 18 corresponding to amino acids 829 to 838 of SEQ ID NO:2; and (x) region 19 corresponding to amino acids 1043 to 1055 of SEQ ID NO:2;

further preferably said detergent component comprises a chelator; further most preferably said chelator is EDTA or citrate.

13. The detergent composition comprising an endoglucanase variant of any of paragraphs 1-12, wherein in an aqueous solution comprising a detergent component said region (e.g. of SEQ ID NO:2 or another parent endoglucanase) selected from the group consisting of regions 1-9 is more accessible to said detergent component than one or more or all of its adjacent regions;

preferably said adjacent region is selected from the group consisting of: (i) region 10 corresponding to amino acids 1 to 94 of SEQ ID NO:2; (ii) region 11 corresponding to amino acids 106 to 114 of SEQ ID NO:2; (iii) region 12 corresponding to amino acids 139 to 209 of SEQ ID NO:2; (iv) region 13 corresponding to amino acids 252 to 266 of SEQ ID NO:2; (v) region 14 corresponding to amino acids 302 to 338 of SEQ ID NO:2; (vi) region 15 corresponding to amino acids 362 to 546 of SEQ ID NO:2; (vii) region 16 corresponding to amino acids 596 to 611 of SEQ ID NO:2; (viii) region 17 corresponding to amino acids 661 to 805 of SEQ ID NO:2; (ix) region 18 corresponding to amino acids 829 to 838 of SEQ ID NO:2; and (x) region 19 corresponding to amino acids 1043 to 1055 of SEQ ID NO:2;

further preferably said detergent component comprises a chelator; further most preferably said chelator is the EDTA or citrate.

14. The detergent composition comprising an endoglucanase variant of any of paragraphs 1-13, wherein in an aqueous solution comprising a detergent component said region (e.g. of SEQ ID NO:2 or another parent endoglucanase) selected from the group consisting of regions 1-9 is more conformationally dynamic than one or more or all of its adjacent regions;

preferably said adjacent region is selected from the group consisting of: (i) region 10 corresponding to amino acids 1 to 94 of SEQ ID NO:2; (ii) region 11 corresponding to amino acids 106 to 114 of SEQ ID NO:2; (iii) region 12 corresponding to amino acids 139 to 209 of SEQ ID NO:2; (iv) region 13 corresponding to amino acids 252 to 266 of SEQ ID NO:2; (v) region 14 corresponding to amino acids 302 to 338 of SEQ ID NO:2; (vi) region 15 corresponding to amino acids 362 to 546 of SEQ ID NO:2; (vii) region 16 corresponding to amino acids 596 to 611 of SEQ ID NO:2; (viii) region 17 corresponding to amino acids 661 to 805 of SEQ ID NO:2; (ix) region 18 corresponding to amino acids 829 to 838 of SEQ ID NO:2; and (x) region 19 corresponding to amino acids 1043 to 1055 of SEQ ID NO:2;

further preferably said detergent component comprises a chelator; further most preferably said chelator is EDTA or citrate.

15. The detergent composition comprising an endoglucanase variant of any of paragraphs 1-14, wherein in an aqueous solution comprising a detergent component said region (e.g. of SEQ ID NO:2 or another parent endoglucanase) selected from the group consisting of regions 1-9 is more receptive to deuterium incorporation than one or more or all of its adjacent regions;

preferably said adjacent region is selected from the group consisting of: (i) region 10 corresponding to amino acids 1 to 94 of SEQ ID NO:2; (ii) region 11 corresponding to amino acids 106 to 114 of SEQ ID NO:2; (iii) region 12 corresponding to amino acids 139 to 209 of SEQ ID NO:2; (iv) region 13 corresponding to amino acids 252 to 266 of SEQ ID NO:2; (v) region 14 corresponding to amino acids 302 to 338 of SEQ ID NO:2; (vi) region 15 corresponding to amino acids 362 to 546 of SEQ ID NO:2; (vii) region 16 corresponding to amino acids 596 to 611 of SEQ ID NO:2; (viii) region 17 corresponding to amino acids 661 to 805 of SEQ ID NO:2; (ix) region 18 corresponding to amino acids 829 to 838 of SEQ ID NO:2; and (x) region 19 corresponding to amino acids 1043 to 1055 of SEQ ID NO:2;

further preferably said detergent component comprises a chelator; further most preferably said chelator is EDTA or citrate.

16. The detergent composition comprising an endoglucanase variant of any of paragraphs 1-15, further comprising an alteration (e.g. a substitution, deletion or insertion, preferably a substitution) at one or more positions in:

a) one or more regions selected from the group consisting of:

i) region 1 corresponding to amino acids 95 to 105 of SEQ ID NO:2, e.g. said alteration at one or more positions selected from the group consisting of positions: 95, 96, 97, 98, 99, 100, 101, 102, 103, 104, 105, wherein said positions correspond to amino acid positions of SEQ ID NO:2 (e.g. using the numbering of SEQ ID NO:2),

ii) region 2 corresponding to amino acids 115 to 138 of SEQ ID NO:2, e.g. said alteration at one or more positions selected from the group consisting of positions: 115, 116, 117, 118, 119, 120, 121, 122, 123, 124, 125, 126, 127, 128, 129, 130, 131, 132, 133, 134, 135, 136, 137, 138, wherein said positions correspond to amino acid positions of SEQ ID NO:2 (e.g. using the numbering of SEQ ID NO:2),

iii) region 3 corresponding to amino acids 210 to 251 of SEQ ID NO:2, e.g. said alteration at one or more positions selected from the group consisting of positions: 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, wherein said positions correspond to amino acid positions of SEQ ID NO:2 (e.g. using the numbering of SEQ ID NO:2),

iv) region 4 corresponding to amino acids 267 to 301 of SEQ ID NO:2, e.g. said alteration at one or more positions selected from the group consisting of positions: 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, wherein said positions correspond to amino acid positions of SEQ ID NO:2 (e.g. using the numbering of SEQ ID NO:2),

v) region 5 corresponding to amino acids 339 to 361 of SEQ ID NO:2, e.g. said alteration at one or more positions selected from the group consisting of positions: 339, 340, 341, 342, 343, 344, 345, 346, 347, 348, 349, 350, 351, 352, 353, 354, 355, 356, 357, 358, 359, 360, 361, wherein said positions correspond to amino acid positions of SEQ ID NO:2 (e.g. using the numbering of SEQ ID NO:2),

vi) region 6 corresponding to amino acids 547 to 595 of SEQ ID NO:2, e.g. said alteration at one or more positions selected from the group consisting of positions: 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, wherein said positions correspond to amino acid positions of SEQ ID NO:2 (e.g. using the numbering of SEQ ID NO:2),

vii) region 7 corresponding to amino acids 612 to 660 of SEQ ID NO:2, e.g. said alteration at one or more positions selected from the group consisting of positions: 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, wherein said positions correspond to amino acid positions of SEQ ID NO:2 (e.g. using the numbering of SEQ ID NO:2),

viii) region 8 corresponding to amino acids 806 to 828 of SEQ ID NO:2, e.g. said alteration at one or more positions selected from the group consisting of positions: 806, 807, 808, 809, 810, 811, 812, 813, 814, 815, 816, 817, 818, 819, 820, 821, 822, 823, 824, 825, 826, 827, 828, wherein said positions correspond to amino acid positions of SEQ ID NO:2 (e.g. using the numbering of SEQ ID NO:2), and

ix) region 9 corresponding to amino acids 839 to 1042 of SEQ ID NO:2, e.g. said alteration at one or more positions selected from the group consisting of positions: 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, wherein said positions correspond to amino acid positions of SEQ ID NO:2 (e.g. using the numbering of SEQ ID NO:2); and/or

b) an adjacent region (e.g. an alteration at one or more positions corresponding to positions: 51 (e.g, K51Q), 451 (e.g. K451S), 333 (e.g. W333L), 416 (e.g. Q416D)), preferably said adjacent region is selected from the group consisting of: (i′) region 10 corresponding to amino acids 1 to 94 of SEQ ID NO:2; (ii′) region 11 corresponding to amino acids 106 to 114 of SEQ ID NO:2; (iii′) region 12 corresponding to amino acids 139 to 209 of SEQ ID NO:2; (iv′) region 13 corresponding to amino acids 252 to 266 of SEQ ID NO:2; (v′) region 14 corresponding to amino acids 302 to 338 of SEQ ID NO:2; (vi′) region 15 corresponding to amino acids 362 to 546 of SEQ ID NO:2; (vii′) region 16 corresponding to amino acids 596 to 611 of SEQ ID NO:2; (viii′) region 17 corresponding to amino acids 661 to 805 of SEQ ID NO:2; (ix′) region 18 corresponding to amino acids 829 to 838 of SEQ ID NO:2; and (x′) region 19 corresponding to amino acids 1043 to 1055 of SEQ ID NO:2,

wherein said variant has at least 60%%, e.g. at least 65%, 70%, 75%, 80%, 85%, 90%, 95%, 96%, 97%, 98%, or 99%, and less than 100% sequence identity to SEQ ID NO:2, preferably said variant has activity on xanthan gum pretreated with xanthan lyase, further preferably said activity is a xanthan gum degrading activity.

17. The detergent composition comprising an endoglucanase variant of any of paragraphs 1-16, wherein said variant has at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:2.

18. The detergent composition comprising an endoglucanase variant of any one of paragraphs 1-17, wherein said alteration at one or more position is selected from the group consisting of alterations in positions: 4, 17, 18, 20, 51, 53, 55, 56, 60, 63, 71, 79, 87, 92, 99, 120, 125, 126, 130, 137, 182, 186, 189, 192, 213, 216, 221, 226, 228, 230, 231, 232, 233, 235, 240, 243, 247, 249, 278, 279, 281, 283, 285, 289, 292, 294, 298, 302, 311, 313, 333, 346, 353, 358, 386, 387, 388, 390, 403, 408, 410, 416, 441, 448, 451, 471, 472, 476, 489, 507, 512, 515, 538, 555, 556, 557, 558, 559, 560, 561, 562, 563, 564, 567, 568, 570, 575, 578, 579, 580, 581, 583, 589, 590, 591, 592, 593, 595, 598, 599, 602, 603, 605, 607, 609, 616, 627, 630, 631, 635, 636, 638, 639, 640, 641, 642, 643, 644, 651, 676, 683, 688, 690, 694, 698, 699, 706, 711, 713, 1719, 720, 744, 749, 754, 756, 760, , 781, 786, 797, 810, 811, 812, 815, 823, 824, 825, 827, 828, 833, 834, 835, 837, 843, 848, 868, 869, 870, 871, 872, 873, 874, 880, 881, 883, 884, 885, 887, 888, 890, 892, 894, 898, 905, 906, 912, 920, 921, 924, 926, 927, 928, 932, 933, 934, 935, 937, 938, 939, 940, 941, 942, 943, 946, 948, 950, 952, 953, 954, 956, 957, 960, 966, 971, 972, 980, 989, 991, 994, 995, 998, 999, 1006, 1009, 1010, 1011, 1029, 1030, 1031, 1032, 1035, 1037, 1038, 1040, 1041, 1042, 1044, 1045, 1048, wherein numbering is according to SEQ ID NO:2.

19. The detergent composition comprising an endoglucanase variant of any of paragraphs 1-18, wherein said alteration at one or more positions is selected from the group consisting of alterations in positions: 285, 333, 353, *558, 558, 633, 635, 635, 635, 638, 639, 994, 281, 563, 575, 575, 921, 558+559+560+561+562, 558, 559, 560, 561, 562 125, 126, 130, 213, 221, 228, 228, 230, 230, 230, 230, 230, 230, 230, 231, 231, 232, 232, 235, 240, 243, 243, 249, 278, 281, 281, 281, 281, 281, 281, 281, 285, 285, 285, 285, 285, 285, 285, 285, 285, 285, 285, 292, 292, 292, 292, 292, 292, 292, 292, 292, 292, 292, 292, 297, 346, 556, 558, 558, 558, 558, 558, 558, 559, 559, 559, 559, 559, 559, 559, 559, 559, 559, 559, 559, 560, 560, 560, 560, 561, 561, 561, 561, 561, 564, 564, 564, 564, 564, 564, 565, 567, 568, 569, 569, 569, 569, 570, 570, 570, 570, 570, 570, 570, 570, 570, 570, 570, 570, 570, 570, 570, 570, 570, 575, 575, 576, 576, 576, 578, 579, 579, 580, 583, 589, 590, 590, 590, 591, 592, 593, 593, 593, 593, 593, 593, 593, 593, 616, 627, 627, 627, 627, 627, 627, 627, 630, 630, 630, 635, 635, 635, 635, 635, 635, 635, 636, 636, 636, 636, 636, 636, 636, 636, 638, 638, 638, 638, 638, 639, 639, 639, 639, 639, 639, 639, 639, 639, 641, 642, 642, 643, 643, 643, 644, 651, 810, 811, 812, 812, 812, 812, 812, 815, 815, 815, 815, 815, 823, 824, 825, 825, 825, 825, 827, 827, 827, 843, 870, 870, 870, 870, 870, 870, 870, 870, 870, 870, 870, 870, 870, 870, 870, 870, 871, 871, 871, 871, 871, 871, 871, 871, 871, 871, 871, 872, 872, 872, 872, 872, 872, 872, 872, 872, 872, 872, 872, 872, 873, 873, 874, 874, 874, 874, 874, 874, 874, 874, 881, 883, 884, 885, 885, 885, 887, 887, 887, 887, 887, 894, 920, 921, 921, 932, 933, 933, 934, 934, 934, 934, 934, 934, 934, 934, 934, 935, 937, 937, 937, 937, 937, 937, 937, 938, 939, 939, 940, 941, 941, 941, 942, 942, 943, 943, 950, 950, 950, 952, 952, 953, 954, 960, 964, 964, 966, 966, 971, 974, 974, 989, 991, 991, 991, 991, 991, 991, 991, 995, 995, 995, 995,995,998,998,1006,1006,1006,1006,1010,1011,1011,1011,1011,1011,1011,1011,1029, 1030,1031,1031,1031,1031,1032,1035,1037,1037,1038,1038,1040,1040,1041,1044,1044, 1044,1044,1045,1045, 559+579, 559,579, 564+579, 564,579, 559+579, 559,579, 562+579, 562, 579, 564+579, 564,579, 559+579+99, 559,579,99, 559+579+281, 559,579,281,281+559+579, 281, 559,579, 559+579+616, 559,579,616, 559+579+636, 559,579, 636, 559+579+651, 559,579, 651, 559+579+948, 559,579,948, 559+579+1009, 559,579,1009, 559+579+627, 559,579,627, 579+921,579,921, 559+579+921, 559,579,921,99+579,99,579,579+651,579,651,579+948, 579,948,579+1009,579,1009, 559+579+934, 559,579,934, 559+579+921+934, 559,579,921, 934, 559+579+627, 559,579,627, 559+579+627+616, 559,579,627,616, 559+579+627, 559,579, 627, 559+579+921+651, 559,579,921,651, 559+579+921+627, 559,579,921,627, 559+579+921+636, 559,579,921, 636, 559+579+921+616, 559,579,921,616, 559+579+921+636, 559,579,921, 636, 559+579+921+627+636, 559,579,921,627, 636, 559+579+636+651, 559,579, 636,651, 559+579+616+651, 559,579,616,651, 559+579+616+636, 559,579,616, 636, 559+579+616+921+934, 559,579,616,921,934, 559+579+651+627, 559,579,651,627, 559+579+651+636, 559,579,651, 636, 559+579+651+627+636, 559,579,651,627, 636, 559+579+651+616, 559,579,651,616, 559+579+651+921+934, 559,579,651,921,934, 636+934, 636,934, 636+921, 636,921, 636+627, 636,627, 636+579, 636,579, 638+934, 638,934, 638+921, 638,921, 638+627, 638,627, 638+579, 638,579,627+51,627, 51,627+451,627, 451,627+559, 627, 559, 627+579, 627, 579, 579+934, 579, 934, 651+638, 651, 638, 570+651, 570, 651, 570+921, 570, 921, 570+627, 570, 627, 570+559, 570, 559, 570+579, 570, 579, 570+638, 570, 638, 570+579, 570, 579, 570+638, 570, 638, 570+651, 570, 651, 570+636, 570, 636, 570+934, 570, 934, 570+638, 570, 638, 570+921, 570, 921, 570+627, 570, 627, 570+559, 570, 559, 570+885, 570, 885, 885+934, 885, 934, 885+627, 885, 627, 559+579+636, 559, 579, 636, 559+579+638, 559, 579, 638, 559+579+870, 870, 559+579+560, 560, 559+579+564, 564, 559+579+570, 570, 559+579+570, 570, 559+579+570, 570, 559+579+570, 570, 559+579+570, 570, 559+579+570, 570, 559+579+570, 570, 559+579+570, 570, 558, 559, 559, 559, 561, 564, 570, 570, 570, 570, 570, 570, 570, 579, 579, 581, 616, 627, 627, 627, 636, 636, 636, 636, 636, 636, 638, 638, 643, 651, 651, 885, 885, 921, 934, 934, 966, 1011, 1031, 559+570+579, 559+570+579, 559+570+579, 559+570+579, 559+570+579, 559+570+579, 559+570+579, 559+570+579, 559+560+579, 559+579+651, 559+579+651+934, 559+579+638, 559+579+921, 559+579+616+921, 559+579+636, 559+579, 559+579, 559+579+921, 559+579+616, 638+934, 627+636, 627+934, 570+579, 416+559+579+636, 416, 128+559+579+627, 128, 128+559+579+636, 579+636 of SEQ ID NO:2, preferably numbering is according to SEQ ID NO:2, further preferably alterations in positions: 627, 636 or 638, wherein numbering is according to SEQ ID NO:2.

20. The detergent composition comprising an endoglucanase variant of any of paragraphs 1-17, wherein said alteration at one or more positions is selected from the group consisting of: N285G, W333L, T353D, N558NP, N558F, T633V, D635L, D635M, D635T, F638Y, T639D, G994N, and K281T, G563E, I575M, I575A, K921D, N558K+A559K+S560F+T561P+G562W, N558K, A559K, S560F, T561P, G562W and I125V, A126R, K130R, K213R, A221R, K228E, K2281, G230F, G230L, G230A, G230H, G230N, G230W, G230T, F231Y, F231N, V232R, V232G, H235D, N240Q, G243K, G243R, A249N, A278S, K281F, K281V, K281Y, K281H, K281Q, K281N, K281W, N285L, N285M, N285S, N285P, N285T, N285Y, N285H, N285K, N285D, N285W, N285R, T292F, T292L, T292I, T292V, T292S, T292P, T292Y, T292Q, T292N, T292K, T292D, T292G, F297L, A346H, G556S, N558D, N558M, N558Q, N5581, N558Y, N558H, A559N, A559F, A559M, A559P, A559Y, A559H, A559Q, A559D, A559R, A559G, A5591, A559S, S560P, S560K, S560G, S560D, T561P, T561E, T561Q, T561S, T561D, A5641, A564Y, A564H, A564Q, A564K, A564E, E565M, V567F, K568R, L569F, L569Y, L569D, L569E, P570F, P570L, P570I, P570M, P570V, P570S, P570T, P570A, P570Y, P570H, P570Q, P570N, P570K, P570E, P570W, P570R, P570G, 1575D, I575E, I576F, I576M, I576P, D578R, Y579F, Y579W, V580L, D583M, Q589G, P590S, P590T, P590E, E591L, G592D, S593P, S593H, S593Q, S593N, S593K, S593D, S593E, S593R, S616D, K627L, K627M, K627V, K627S, K627T, K627Q, K627R, I630F, I630V, I630Y, D635A, D635P, D635N, D635K, D635E, D635G, D635W, S636L, S636M, S636A, S636H, S636Q, S636N, S636K, S636R, F6381, F638V, F638T, F638L, F638H, T639V, T639S, T639L, T6391, T639M, T639A, T639E, T639W, T639G, Y641E, S642T, S642N, N643D, N643H, N643T, T644F, A651P, S810R, A811S, V812F, V812I, V812M, V812W, V812R, N815V, N815Y, N815E, N815W, N815R, S823Q, A824T, T825N, T825W, T825A, T825D, V827I, V827M, V827S, T843V, D870F, D870L, D870I, D870M, D870V, D870S, D870T, D870Y, D870H, D870Q, D870N, D870K, D870E, D870W, D870R, D870G, P871F, P871L, P871I, P871M, P871V, P871S, P871T, P871A, P871Y, P871H, P871Q, T872S, T872F, T872A, T872Y, T872H, T872Q, T872N, T872K, T872D, T872E, T872W, T872R, T872G, D873K, D873E, T874V, T874S, T874P, T874A, T874H, T874Q, T874N, T874K, V881Q, T883K, Y884H, A885F, A885Q, A885N, T887L, T887I, T887S, T887H, T887R, K894E, N920D, K921R, K921E, T932A, N933V, N933S, Y934G, Y934M, Y934S, Y934A, Y934Q, Y934N, Y934E, Y934W, Y934R, T935W, A937F, A937V, A937S, A937T, A937Q, A937D, A937E, V9381, K939I, K939V, D940E, N941S, N941H, N941D, A942P, A942E, D943Y, D943H, R950V, R950H, R950N, F952S, F952W, N953Y, G954L, Y960F, A964N, A964C, N966P, N966C, G971A, Q974K, Q974C, Q9891, Q991L, Q991I, Q991M, Q991V, Q991T, Q991K, Q991C, S995I, S995V, S995Q, S995R, S995C, G998V, G998A, S1006T, S1006A, S1006K, S1006R, Y1010W, L1011M, L1011S, L1011A, L1011Q, L1011N, L1011D, L1011E, R1029N, F1030M, K10311, K1031S, K1031T, K1031H, V1032G, K1035A, A1037E, A1037W, S1038L, S1038I, L1040N, L1040E, G1041F, L1044F, L1044S, L1044N, L1044W, P1045Q, P1045W, and A559N+Y579F, A559N, Y579F, A564E+Y579F, A564E, Y579F, A559N+Y579W, A559N, Y579W, G562P+Y579W, G562P, Y579W, A564D+Y579W, A564D, Y579W, A559N+Y579W+K99R, A559N, Y579W, K99R, A559N+Y579W+K281R, A559N, Y579W, K281R, K281R+A559N+Y579W, K281R, A559N, Y579W, A559N+Y579W+S616D, A559N, Y579W, S616D, A559N+Y579W+S636N, A559N, Y579W, S636N, A559N+Y579W+A651P, A559N, Y579W, A651P, A559N+Y579W+K948E, A559N, Y579W, K948E, A559N+Y579W+K1009E, A559N, Y579W, K1009E, A559N+Y579W+K627R, A559N, Y579W, K627R, Y579W+K921R, Y579W, K921R, A559N+Y579W+K921R, A559N, Y579W, K921R, K99R+Y579W, K99R, Y579W, Y579W+A651P, Y579W, A651P, Y579W+K948E, Y579W, K948E, Y579W+K1009E, Y579W, K1009E, A559N+Y579W+Y934G, A559N, Y579W, Y934G, A559N+Y579W+K921R+Y934G, A559N, Y579W, K921R, Y934G, A559N+Y579W+K627M, A559N, Y579W, K627M, A559N+Y579W+K627R+S616D, A559N, Y579W, K627R, S616D, A559N+Y579F+K627R, A559N, Y579F, K627R, A559N+Y579W+K921R+A651P, A559N, Y579W, K921R, A651P, A559N+Y579W+K921R+K627R, A559N, Y579W, K921R, K627R, A559N+Y579W+K921R+S636K, A559N, Y579W, K921R, S636K, A559N+Y579W+K921R+S616D, A559N, Y579W, K921R, S616D, A559N+Y579W+K921R+S636N, A559N, Y579W, K921R, S636N, A559N+Y579W+K921R+K627R+S636N, A559N, Y579W, K921R, K627R, S636N, A559N+Y579W+S636N+A651P, A559N, Y579W, S636N, A651P, A559N+Y579W+S616D+A651P, A559N, Y579W, S616D, A651P, A559N+Y579W+S616D+S636K, A559N, Y579W, S616D, S636K, A559N+Y579W+S616D+K921R+Y934G, A559N, Y579W, S616D, K921R, Y934G, A559N+Y579W+A651P+K627M, A559N, Y579W, A651P, K627M, A559N+Y579W+A651P+S636K, A559N, Y579W, A651P, S636K, A559N+Y579W+A651P+K627R+S636N, A559N, Y579W, A651P, K627R, S636N, A559N+Y579W+A651P+S616D, A559N, Y579W, A651P, S616D, A559N+Y579W+A651P+K921R+Y934G, A559N, Y579W, A651P, K921R, Y934G, S636N+Y934G, S636N, Y934G, S636N+K921R, S636N, K921R, S636N+K627R, S636N, K627R, S636N+Y579W, S636N, Y579W, F6381+Y934G, F638I, Y934G, F638I+K921R, F638I, K921R, F6381+K627R, F638I, K627R, F6381+Y579W, F6381, Y579W, K627R+K51Q, K627R, K51Q, K627R+K451S, K627R, K451S, K627R+A559N, K627R, A559N, K627R+Y579W, K627R, Y579W, Y579W+Y934G, Y579W, Y934G, A651P+F638I, A651P, F638I, P570Q+A651P, P570Q, A651P, P570Q+K921R, P570Q, K921R, P570Q+K627R, P570Q, K627R, P570Q+A559N, P570Q, A559N, P570Q+Y579W, P570Q, Y579W, P570Q+F638I, P570Q, F638I, P570K+Y579W, P570K, Y579W, P570K+F6381, P570K, F638I, P570T+A651P, P570T, A651P, P570T+S636N, P570T, S636N, P570T+Y934G, P570T, Y934G, P570T+F638I, P570T, F638I, P570T+K921R, P570T, K921R, P570T+K627R, P570T, K627R, P570T+A559N, P570T, A559N, P570T+A885F, P570T, A885F, A885F+Y934G, A885F, Y934G, A885F+K627R, A885F, K627R, A559N+Y579W+S636L, A559N, Y579W, S636L, A559N+Y579W+F638I, A559N, Y579W, F638I, A559N+Y579W+D870M, D870M, A559N+Y579W+S560P, 5560P, A559N+Y579W+A564I, A564I, A559N+Y579W+P570N, P570N,

A559N+Y579W+P570K, P570K, A559N+Y579W+P570R, P570R, A559N+Y579W+P570A, P570A, A559N+Y579W+P570T, P570T, A559N+Y579W+P570S, P570S, A559N+Y579W+P570Q, P570Q, A559N+Y579W+P570H, P570H, and N558E, A559P, A559N, A559H, T561P, A564E, P570A, P570Q, P570R, P570S, P570K, P570T, P570N, Y579W, Y579F, T581M, S616D, K627R, K627M, K627Q, S636N, S636Q, S636R, S636K, S636M, S636H, F638I, F638L, N643D, A651P, A651S, A885F, A885Q, K921R, Y934R, Y934G, N966C, L1011A, K1031I, and A559N+P570A+Y579W, A559N+P570H+Y579W, A559N+P570K+Y579W, A559N+P570N+Y579W, A559N+P570Q+Y579W, A559N+P570R+Y579W, A559N+P570K+Y579W, A559N+P570T+Y579W, A559N+S560P+Y579W, A559N+Y579W+A651P, A559N+Y579W+A651P+Y934G, A559N+Y579W+F638I, A559N+Y579W+K921R, A559N+Y579W+S616D+K921R, A559N+Y579W+S636N, A559N+Y579F, A559N+Y579W, A559N+Y579W+K921R, A559N+Y579W+S616D, F638I+Y934G, K627R+S636N, K627R+Y934G, P570K+Y579W, Q416D+A559N+Y579W+S636N, Q416D, S128X+A559N+Y579W+K627R, S128X, S128X+A559N+Y579W+S636N, Y579W+S636N, V4T, S17A, N18G, F20P, F20N, F20G, F20Y, K51Q, K51H, E53Y, E53P, E53G, Y55M, Y55D, V56M, Y60F, S63F, A71E, 579W, T87R, T92S, A120P, N129D, F137L, H182Y, A186P, N189K, K192N, N216D, N216Q, N216R, L226K, G230H, L233H, D247N, G279E, K281R, A283D, N285D, N285G, Q289E, T292A, T292F, T292Y, A294V, Q298E, I302D, I302H, I302V, I302M, H311N, S313D, A346D, A386P, I387T, K388R, K390Q, 1403Y, E408D, E408N, E4085, E408P, E408A, E408G, P410G, Q4165, Q416D, N441G, A448E, A448W, A4485, K451S, K451Q, G471S, S472Y, D476R, Q489P, K507R, K512P, S515V, S538C, L555Q, G557R, N558E, A559N, A559P, A559H, A559D, S560P, S560G, T561P, A564E, A5641, V567P, K568R, P570R, P570Q, P570K, P570A, P570T, P570G, P570S, P570H, P570N, I575V, Y579W, Y579F, T581M, S593N, S593E, S595L, S598Q, A599S, I602T, I602D, V603P, S605T, S607C, G609E, S616G, S616D, K627R, K627M, K627Q, K631R, K631A, D635A, D635E, D635M, D635N, D635L, D635W, S636N, S636K, S636L, S636Q, S636R, S636M, 5636H, F638N, F638I, F638L, F638V, F638H, F638M, T639G, T639I, T639M, T639Y, T639W, T639P, T639E, T640S, S642N, S642T, N643D, N643H, A651P, A651S, D676H, Q683E, A688G, Y690F, T694A, T697G, R698W, T699A, T706Q, T711S, T711V, T711Y, K713R, W719R, K720H, K744H, K744Q, A749T, K754R, V756Y, V756H, S760G, T, 781M, N786K, T797S, S810Q, A824D, T825G, N828D, N833D, Q834E, S835A, S835D, V837I, N848D, A868E, A869V, D870V, T872G, T872H, T872W, T872Q, R880K, V881Q, V881T, T883R, T883V, T883C, T883K, Y884H, A885N, A885Q, A885F, T887K, T887S, L888M, V890R, T892P, T892V, R898Q, N905D, F906A, Q912V, N920P, K921R, A924D, V926F, V926P, K927R, S928D, T932A, N933S, N933V, Y934G, Y934R, Y934Q, A937E, V938I, K939V, N941S, A942P, G946R, K948R, Q956Y, Q956S, A957L, A957P, N966C, T972K, M9801, G994D, T999R, L1011A, K1031I, A1037E, S1038G, G1041R, Y1042N, F1048W, preferably numbering is according to SEQ ID NO:2, further preferably alterations in positions: K627R, S636N or F6381, wherein numbering is according to SEQ ID NO:2.

21. The detergent composition comprising an endoglucanase variant of any of paragraphs 1-20, wherein said alteration at one or more positions is selected from the group consisting of alterations in positions: 17, 20, 51, 53, 55, 56, 60, 63, 79, 87, 192, 302, 387, 388, 390, 403, 408, 410, 416, 448, 451, 471, 472, 507, 512, 515, 538, 598, 602, 605, 609, 676, 694, 698, 699, 711, 754, 760, , 781, 786, 797, 834, and 835 of SEQ ID NO:2, wherein numbering is according to SEQ ID NO:2.

22. The detergent composition comprising an endoglucanase variant of any of paragraphs 1-21, wherein said alteration at one or more positions is selected from the group consisting of: 517A, F20P, F20N, F20G, F20Y, K51Q, K51H, E53P, E53G, Y55M, V56M, Y60F, S63F, T87R, K192N, I302H, I302V, I302M, I387T, K388R, K390Q, I403Y, E408D, E408S, E408P, E408A, E408G, E408N, P410G, Q416S, Q416D, A448E, A448W, A4485, K451S, G471S, S472Y, K507R, K512P, S515V, S538C, Y579W, S598Q, I602T, I602D, S605T, G609E, D676H, T694A, R698W, T699A, T711V, T711Y, K754R, 5760G, T, 781M, N786K, T797S, Q834E, and S835D of SEQ ID NO:2.

23. The detergent composition comprising an endoglucanase variant of any of paragraphs 1-22, wherein the total number of alterations compared to the parent endoglucanase (e.g. SEQ ID NO:2) is between 1 and 20, e.g. between 1 and 18 or between 5 and 16 or from 8 to 14, such as 1, 2, 3, 4, 5, 6, 7, 8, 9, 10, 11, 12, 13, 14, 15, or 16 alterations.

24. The detergent composition comprising an endoglucanase variant of any of paragraphs 1-23, wherein said activity on xanthan gum pretreated with xanthan lyase is a xanthan degrading activity, preferably said xanthan degrading activity is endoglucanase EC 3.2.1.4 activity.

25. The detergent composition comprising an endoglucanase variant of any of paragraphs 1-24, wherein said variant has an improved stability in a detergent composition compared to a parent endoglucanase (e.g. with SEQ ID NO:2); optionally said detergent composition comprises a chelator; further preferably said chelator is EDTA or citrate.

26. The detergent composition comprising an endoglucanase variant of any of paragraphs 1-25, wherein said variant has a half-life improvement factor (HIF) of 1.0; preferably said variant has a half-life improvement factor (HIF) of >1.0 relative to a parent endoglucanase, e.g. an endoglucanase of SEQ ID NO:2.

27. The detergent composition comprising an endoglucanase variant of paragraph 24, wherein said half-life improvement factor (HIF) is determined after incubation of said endoglucanase variant in a detergent composition at 25° C. for a time period from about 5-140 h or from about 17-20 h.

28. The detergent composition of any one of paragraphs 1-27, wherein the xanthan lyase variant is a variant of a parent xanthan lyase selected from the group consisting of: a) a polypeptide having at least 60% sequence identity to the mature polypeptide of SEQ ID NO:6; b) a polypeptide encoded by a polynucleotide that hybridizes under low stringency conditions with (i) the mature polypeptide coding sequence of SEQ ID NO:5, or (ii) the full-length complement of (i); c) a polypeptide encoded by a polynucleotide having at least 60% identity to the mature polypeptide coding sequence of SEQ ID NO:5; and d) a fragment of the mature polypeptide of SEQ ID NO:6, which has xanthan lyase activity.

29. The detergent composition of paragraph 28, wherein the parent xanthan lyase has at least 60%, e.g. at least 65%, 70%, 75%, 80%, 85%, 90%, 95%, 96%, 97%, 98%, 99% or 100% sequence identity to the mature polypeptide of SEQ ID NO:6.

30. The detergent composition of any of paragraphs 28-29, wherein the parent xanthan lyase is encoded by a polynucleotide that hybridizes under low stringency conditions, medium stringency conditions, medium-high stringency conditions, high stringency conditions, or very high stringency conditions with (i) the mature polypeptide coding sequence of SEQ ID NO:5 or (ii) the full-length complement of (i).

31. The detergent composition of any of paragraphs 28-30, wherein the parent xanthan lyase is encoded by a polynucleotide having at least 60%, e.g. at least 65%, 70%, 75%, 80%, 85%, 90%, 95%, 96%, 97%, 98%, 99%, or 100% sequence identity to the mature polypeptide coding sequence of SEQ ID NO:5.

32. The detergent composition of any of paragraphs 28-31, wherein the parent xanthan lyase comprises or consists of the mature polypeptide of SEQ ID NO:6.

33. The detergent composition of any of paragraphs 28-32, wherein the parent xanthan lyase is a fragment of the mature polypeptide of SEQ ID NO:6, wherein the fragment has xanthan lyase activity.

34. The detergent compositionof any of paragraphs 28-33, wherein the xanthan lyase variant has at least 60%, e.g. at least 65%, 70%, 75%, 80%, 85%, 90%, 95%, 96%, 97%, 98%, or 99%, but less than 100%, sequence identity to the amino acid sequence of the parent xanthan lyase.

35. The detergent composition of any of paragraphs 1-34, wherein said region of the xanthan lyase selected from the group consisting of regions 1-6 is a chelator-induced instability region;

preferably said chelator-induced instability region (e.g. of SEQ ID NO:6 or another parent xanthan lyase) has one or more of the following features: (i) in the presence of a chelator it is less conformationally stable than one or more or all of its adjacent regions; and/or (ii) in the presence of a chelator it is more exposed to said chelator than one or more or all of its adjacent regions; and/or (iii) in the presence of a chelator it is more accessible to said chelator than one or more or all of its adjacent regions; and/or (iv) in the presence of a chelator it is more conformationally dynamic than one or more or all of its adjacent regions; and/or (v) in the presence of a chelator it is more receptive to deuterium incorporation than one or more or all of its adjacent regions;

further preferably said adjacent region is selected from the group consisting of: (vi) region 7 corresponding to amino acids 1 to 153 of SEQ ID NO:6; (vii) region 8 corresponding to amino acids 177 to 613 of SEQ ID NO:6; (viii) region 9 corresponding to amino acids 659 to 730 of SEQ ID NO:6; (ix) region 10 corresponding to amino acids 804 to 806 of SEQ ID NO:6; (x) region 11 corresponding to amino acids 847 to 871 of SEQ ID NO:6; (xi) region 12 corresponding to amino acids 886 to 902 of SEQ ID NO:6; and (xii) region 13 corresponding to amino acids 1005 to 1037 of SEQ ID NO:6;

said chelator optionally being EDTA or citrate.

36. The detergent composition of any of paragraphs 1-35, wherein in an aqueous solution comprising a detergent component said region of the xanthan lyase (e.g. of SEQ ID NO:6 or another parent xanthan lyase) selected from the group consisting of regions 1-6 is less conformationally stable than one or more or all of its adjacent regions;

preferably said adjacent region is selected from the group consisting of: (i) region 7 corresponding to amino acids 1 to 153 of SEQ ID NO:6; (ii) region 8 corresponding to amino acids 177 to 613 of SEQ ID NO:6; (iii) region 9 corresponding to amino acids 659 to 730 of SEQ ID NO:6; (iv) region 10 corresponding to amino acids 804 to 806 of SEQ ID NO:6; (v) region 11 corresponding to amino acids 847 to 871 of SEQ ID NO:6; (vi) region 12 corresponding to amino acids 886 to 902 of SEQ ID NO:6; and (vii) region 13 corresponding to amino acids 1005 to 1037 of SEQ ID NO:6,

said detergent component optionally comprising a chelator; optionally said chelator being EDTA or citrate.

37. The detergent composition of any of paragraphs 1-36, wherein in an aqueous solution comprising a detergent component said region of the xanthan lyase (e.g. of SEQ ID NO:6 or another parent xanthan lyase) selected from the group consisting of regions 1-6 is more exposed to said detergent component than one or more or all of its adjacent regions;

preferably said adjacent region is selected from the group consisting of: (i) region 7 corresponding to amino acids 1 to 153 of SEQ ID NO:6; (ii) region 8 corresponding to amino acids 177 to 613 of SEQ ID NO:6; (iii) region 9 corresponding to amino acids 659 to 730 of SEQ ID NO:6; (iv) region 10 corresponding to amino acids 804 to 806 of SEQ ID NO:6; (v) region 11 corresponding to amino acids 847 to 871 of SEQ ID NO:6; (vi) region 12 corresponding to amino acids 886 to 902 of SEQ ID NO:6; and (vii) region 13 corresponding to amino acids 1005 to 1037 of SEQ ID NO:6,

said detergent component optionally comprising a chelator; said chelator optionally being EDTA or citrate.

38. The detergent composition of any of paragraphs 1-37, wherein in an aqueous solution comprising a detergent component said region of the xanthan lyase (e.g. of SEQ ID NO:6 or another parent xanthan lyase) selected from the group consisting of regions 1-6 is more accessible to said detergent component than one or more or all of its adjacent regions;

preferably said adjacent region is selected from the group consisting of: (i) region 7 corresponding to amino acids 1 to 153 of SEQ ID NO:6, (ii) region 8 corresponding to amino acids 177 to 613 of SEQ ID NO:6, (iii) region 9 corresponding to amino acids 659 to 730 of SEQ ID NO:6, (iv) region 10 corresponding to amino acids 804 to 806 of SEQ ID NO:6, (v) region 11 corresponding to amino acids 847 to 871 of SEQ ID NO:6, (vi) region 12 corresponding to amino acids 886 to 902 of SEQ ID NO:6, and (vii) region 13 corresponding to amino acids 1005 to 1037 of SEQ ID NO:6,

said detergent component optionally comprising a chelator; said chelator optionally being EDTA or citrate.

39. The detergent composition of any of paragraphs 1-38, wherein in an aqueous solution comprising a detergent component said region of the xanthan lyase (e.g. of SEQ ID NO:6 or another parent xanthan lyase) selected from the group consisting of regions 1-6 is more conformationally dynamic than one or more or all of its adjacent regions;

preferably said adjacent region is selected from the group consisting of: (i) region 7 corresponding to amino acids 1 to 153 of SEQ ID NO:6, (ii) region 8 corresponding to amino acids 177 to 613 of SEQ ID NO:6, (iii) region 9 corresponding to amino acids 659 to 730 of SEQ ID NO:6, (iv) region 10 corresponding to amino acids 804 to 806 of SEQ ID NO:6, (v) region 11 corresponding to amino acids 847 to 871 of SEQ ID NO:6, (vi) region 12 corresponding to amino acids 886 to 902 of SEQ ID NO:6, and (vii) region 13 corresponding to amino acids 1005 to 1037 of SEQ ID NO:6,

said detergent component optionally comprising a chelator; said chelator optionally being EDTA or citrate.

40. The detergent composition of any of paragraphs 1-39, wherein in an aqueous solution comprising a detergent component said region of the xanthan lyase (e.g. of SEQ ID NO:6 or another parent xanthan lyase) selected from the group consisting of regions 1-6 is more receptive to deuterium incorporation than one or more or all of its adjacent regions;

preferably said adjacent region is selected from the group consisting of: (i) region 7 corresponding to amino acids 1 to 153 of SEQ ID NO:6, (ii) region 8 corresponding to amino acids 177 to 613 of SEQ ID NO:6, (iii) region 9 corresponding to amino acids 659 to 730 of SEQ ID NO:6, (iv) region 10 corresponding to amino acids 804 to 806 of SEQ ID NO:6, (v) region 11 corresponding to amino acids 847 to 871 of SEQ ID NO:6, (vi) region 12 corresponding to amino acids 886 to 902 of SEQ ID NO:6, and (vii) region 13 corresponding to amino acids 1005 to 1037 of SEQ ID NO:6,

said detergent component optionally comprising a chelator; said chelator optionally being EDTA or citrate.

41. The detergent compositionof any of paragraphs 1-40, the xanthan lyase variant comprising an alteration (e.g. a substitution, deletion or insertion, preferably a substitution) at one or more positions in two or more regions selected from the group consisting of: (i) region 1 corresponding to amino acids 154 to 176 of SEQ ID NO:6, (ii) region 2 corresponding to amino acids 614 to 658 of SEQ ID NO:6, (iii) region 3 corresponding to amino acids 731 to 803 of SEQ ID NO:6, (iv) region 4 corresponding to amino acids 807 to 846 of SEQ ID NO:6, (v) region 5 corresponding to amino acids 872 to 885 of SEQ ID NO:6, (vi) region 6 corresponding to amino acids 903 to 1004 of SEQ ID NO:6,

wherein said variant has at least 60%, e.g. at least 65%, 70%, 75%, 80%, 85%, 90%, 95%, 96%, 97%, 98%, or 99%, and less than 100% sequence identity to SEQ ID NO:6, preferably said variant has activity on xanthan gum, further preferably said activity is a xanthan gum degrading activity.

42. The detergent composition of any of paragraphs 1-41, wherein said xanthan lyase variant has at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:6.

43. The detergent composition of any of paragraphs 1-42, wherein said alteration of the xanthan lyase (e.g. a substitution, deletion or insertion, preferably a substitution) at one or more positions is selected from the group consisting of alterations in positions: 155, 159, 620, 624, 626, 631, 635, 645, 649, 650, 656, 738, 745, 746, 748, 752, 753, 754, 757, 764, 769, 774, 775, 777, 779, 782, 785, 786, 789, 792, 796, 799, 800, 801, 819, 824, 843, 845, 875, 903, 911, 912, 915, 919, 921, 923, 925, 927, 928, 930, 932, 933, 941, 966, 967, 991 and 998. of SEQ ID NO:6, wherein numbering is according to SEQ ID NO:6, preferably alterations in positions: 775, 779 or 923, wherein numbering is according to SEQ ID NO:6.

44. The detergent composition of any of paragraphs 1-43, wherein said alteration of the xanthan lyase at one or more positions is selected from the group consisting of: Y155E, A159P, K620R, A624E, A626G, T631N, T631E, S635E, S635T, S635Q, A645S, T649V, T649K, T649R, Q650G, I656V, G738L, K745R, F746L, L748T, P752R, P752K, G753E, G753Q, G753S, S754E, S754L, S754Q, S754R, S757D, S757P, S757E, P764V, P764K, A769D, A769T, A769R, A769S, A769E, A769Q, A769*, A774V, L775M, L775Y, L775A, L7751, L775S, L775F, L775Q, D777K, D777R, P779V, Y782I, A785T, N786K, G789R, K792W, K792Y, K792V, K792A, N796Q, A799H, V800P, D801G, K819R, K819T, K824R, A843P, D845E, K875T, K875E, T903A, T903Q, A911V, A911M, A911S, A912T, A912I, A912Y, T915Q, T915S, T915V, T915A, T919F, T919G, T919D, T921R, T921S, T923H, T923D, T925Q, T925D, T925R, T927K, D928W, Y930H, Y930L, Y930F, A932P, D933M, G941E, G941D, A966P, A967D, N991D and V998K, wherein numbering is according to SEQ ID NO:6.

45. The detergent composition of any of paragraphs 1-44, comprising an alteration of the xanthan lyase at one or more positions in at least one region selected from the group consisting of: (vii) region 7 corresponding to amino acids 1 to 153 of SEQ ID NO:6, (viii) region 8 corresponding to amino acids 177 to 613 of SEQ ID NO:6, (ix) region 9 corresponding to amino acids 659 to 730 of SEQ ID NO:6, (x) region 10 corresponding to amino acids 804 to 806 of SEQ ID NO:6, (xi) region 11 corresponding to amino acids 847 to 871 of SEQ ID NO:6, (xii) region 12 corresponding to amino acids 886 to 902 of SEQ ID NO:6, and (xiii) region 13 corresponding to amino acids 1005 to 1037 of SEQ ID NO:6.

46. The detergent composition of paragraph 45, wherein said alteration of the xanthan lyase at one or more positions in at least one region selected from the group consisting of regions 7, 8, 9, 10, 11, 12 and 13 is an alteration at one or more positions selected from the group consisting of: 9, 15, 18, 46, 58, 66, 89, 95, 100, 106, 109, 183, 188, 190, 203, 204, 221, 229, 234, 238, 240, 242, 243, 257, 258, 284, 291, 293, 316, 317, 320, 324, 329, 333, 339, 341, 352, 354, 360, 372, 377, 399, 400, 419, 440, 450, 451, 454, 458, 481, 492, 505, 533, 567, 568, 576, 578, 579, 582, 664, 672, 703, 722, 726, 727, 728, 851, 855, 856, 867, 887, 892, 899, 900, 901, 902, 915, 1008 and 1016 of SEQ ID NO:6.

47. The detergent composition of paragraph 46, wherein said alteration of the xanthan lyase at one or more positions in at least one region selected from the group consisting of regions 7, 8, 9, 10, 11, 12 and 13 comprises one or more substitutions selected from the group consisting of: K9R, N15T, T18D, L46D, A58L, S66H, Q89Y, K95E, S100D, N106Y, Q109R, Q109D, Q109F, Q109K, Q109A, K183Q, K183R, V188I, A190Q, A203P, K204R, A221P, E229N, E229S, E229V, I234V, I238W, I238L, I238M, I240W, N242S, G243V, Y257W, R258E, R284G, K291R, A293G, A293P, K316R, R317K, K320R, L324Q, K329R, K333R, L339M, I341P, V352I, S354P, K360G, K360R, Q372H, F377Y, N399K, K400R, F419Y, N440K, D450P, K451E, K451R, A454V, D458S, K481R, A492H, A492L, T505I, L533I, K567R, G568A, S578K, S578N, S578R, S579R, S579K, S582K, T664K, N672D, I703L, I722F, P726Q, T727P, M728V, S851F, K855R, E856D, P867S, K887R, N892Y, N892W, N892F, G899S, I1900G, D901A, T902F, N1008D and K1016T of SEQ ID NO:6.

48. The detergent composition of any of paragraphs 1-47, the xanthan lyase variant comprising an alteration at one or more positions selected from the group consisting of positions 624, 631, 635, 649, 656, 738, 752, 753, 754, 757, 769, 775, 777, 800, 801, 843, 875, 911 and 915, and an alteration at one or more positions selected from the group consisting of positions 89, 100, 190, 229, 234, 352, 360, 399, 440, 458, 492, 567, 582, 664, 672, 703, 728, 892, 1008 and 1016 of SEQ ID NO:6.

49. The detergent composition of paragraph 48, the xanthan lyase variant comprising one or more substitutions selected from the group consisting of Q89Y, S100D, A190Q, E229S, 1234V, V352I, K360G, N399K, N440K, D458S, A492H, A492L, K567R, S582K, T664K, N672D, I703L, M728V, N892Y N1008D and K1016T, and one or more substitutions selected from the group consisting of A624E, T631N, S635E, T649K, I656V, G738L, P752K, P752R, G753E, S754E, S754R, S757D, A769D, L775A, D777R, V800P, D801G, A843P, K875T, A911V and T915A.

50. The detergent composition of any of paragraphs 1-49, wherein said alteration of the xanthan lyase (e.g. a substitution, deletion or insertion, preferably a substitution) at one or more positions is selected from the group consisting of alterations in positions: 9, 15, 46, 58, 66, 89, 95, 100, 106, 109, 183, 188, 190, 203, 204, 221, 229, 234, 238, 240, 242, 243, 257, 258, 291, 293, 316, 320, 324, 329, 333, 339, 341, 352, 354, 360, 377, 400, 419, 450, 451, 454, 481, 492, 567, 568, 578, 579, 664, 672, 855, 887 and 892 of SEQ ID NO:6, wherein numbering is according to SEQ ID NO:6.

51. The detergent composition of any of paragraphs 1-50, wherein the xanthan lyase variant has one or more substitutions selected from the group consisting of: K9R, N15T, L46D, A58L, S66H, Q89Y, K95E, S100D, N106Y, Q109R, Q109D, Q109F, Q109K, Q109A, K183Q,K183R, V188I, A190Q, A203P, K204R, A221P, E229N, E229S, I234V, I238W, I238L, I238M, I240W, N242S, G243V, Y257W, R258E, K291R, A293G, A293P, K316R, K320R, L324Q, K329R, K333R, L339M, 1341P, V352I, S354P, K360R, F377Y, K400R, F419Y, D450P, K451E, K451R, A454V, K481R, A492L, K567R, G568A, S578K, S578R, S579R, S579K, T664K, N672D, K855R, K887R, N892Y, N892W and N892F, wherein numbering is according to SEQ ID NO:6.

52. The detergent composition of any of paragraphs 1-51, wherein said xanthan lyase variant does not comprise any amino acid alteration at a position outside of regions 7, 8, 9, 10, 11, 12 and 13.

53. The detergent composition of any of paragraphs 1-52, wherein the total number of alterations of the xanthan lyase compared to the parent xanthan lyase (e.g. SEQ ID NO:6) is from 1 to 20, e.g. from 1 to 18 or from 5 to 17 or from 8 to 16, such as 1, 2, 3, 4, 5, 6, 7, 8, 9, 10, 11, 12, 13, 14, 15, 16, 17 or 18 alterations.

54. The detergent composition of any of paragraphs 1-53, wherein said activity on xanthan gum is a xanthan gum degrading activity, preferably said xanthan lyase variant has EC 4.2.2.12 activity.

55. The detergent composition of any of paragraphs 1-54, wherein said xanthan lyase variant has an improved stability in a detergent composition compared to a parent xanthan lyase (e.g. with SEQ ID NO:6); optionally said detergent composition comprises a chelator; optionally said chelator is EDTA or citrate.

56. The detergent composition of any of paragraphs 1-55, wherein said xanthan lyase variant has a half-life improvement factor (HIF) of 1.0; preferably said variant has a half-life improvement factor (HIF) of >1.0, more preferably at least 1.2, such as at least 1.5, e.g. at least 2.0, relative to a parent xanthan lyase, e.g. a xanthan lyase with SEQ ID NO:6.

57. The detergent composition of paragraph 56, wherein said half-life improvement factor (HIF) is determined after incubation of said xanthan lyase variant in a detergent composition at 25° C. for a time period from about 30 min to about 20 h.

58. The detergent composition of any of paragraphs 1-57, wherein said variant is selected from the group consisting of i) the xanthan lyase variants set forth in Table 17-33 herein and/or xii) the endoglucanase variants set forth in any one of Tables 2-16 herein.

59. The detergent composition of any of paragraphs 1-58, wherein the endoglucanase variant has an alteration (e.g. a substitution, deletion or insertion, preferably a substitution) at one or more positions in: (i) regions 6 and 17; (ii) regions 6, 15 and 17; (iii) regions 10, 12 and 15; (iv) regions 6, 7, 16, and 17; (v) region 6, 9, 10, 12, 15, and 17; (vi) region 14 and 15; (vii) region 9; (viii) 6, 7, 9, 14, 15, 16, and 17; or (ix) 3, 6, 7, 9, 14, 15, 16, and 17; wherein said variant preferably has no alternation in the other regions besides those mentioned.

60. The detergent composition of any of paragraphs 1-59, wherein the endoglucanase variant has an alteration (e.g. a substitution, deletion or insertion, preferably a substitution) in the positions selected from the group consisting of positions: 559+579+697; 512+559+579+697; 18+71+186+408+579+602+651+688+756; 18+189+408+559+579+688+697+756+921+934; 313+488; 880+905+921+934; 302+313+408+579+602+651+697+880+921+934; or 216+313+408+476+579+602+638+651+697+719+880+887+921+934 of SEQ ID NO:2.

61. The detergent composition of paragraph 60, wherein the endoglucanase variant has an alteration (e.g. a substitution, deletion or insertion, preferably a substitution) selected from the group consisting of the following alterations: A559N+Y579W+T697G; K512P+A559N+Y579W+T697G; N18G+A71E+A186P+E408D+Y579W+1602T+A651P+A688G+V756Y; N18G+N189K+E408D+A559N+Y579W+A688G+T697G+V756Y+K921R+Y934G; S313D+E408D; R880K+N905D+K921R+Y934G; I302D+S313D+E408D+Y579W+I602T+A651P+T697G+R880K+K921R+Y934G; and N216Q+5313D+E408D+D476R+Y579W+I602T+F638N+A651P+T697G+W719R+R880K+T887K+K9 21R+Y934G of SEQ ID NO:2.

62. The detergent composition of any one of paragraphs 1 to 61, wherein the xanthan lyase variant comprises an alteration (e.g. a substitution, deletion or insertion, preferably a substitution) at one or more positions in: (i) regions 3 and 5; (ii) regions 3, 5 and 12; (iii) regions 8, and 9; (iv) regions 2, 3, and 5; (v) regions 2, 3, 5, and 12; (vi) regions 3, 5, 8, 9, and 12; (vii) regions 2, 3, 5, 8, and 9; (viii) 3, 5, 8, 9, and 12; (ix) 2, 3, 5, 8, 9, and 12; (x) region 3; (xi) regions 3, 4 and 5; (xii) regions 7, 8 and 9; (xiii) regions 12 and 13; (xiv) regions 3, 4, 5, 8, 9, and 12; (xv) regions 8, 9, 12, and 13; (xvi) regions 7, 8, 9, 12, and 13; (xvii) regions 3, 4, 5, 7, 8, 9, and 12; and (xviii) regions 3, 4, 5, 7, 8, 9, 12, and 13, wherein said variant preferably has no alteration in the other regions besides those mentioned.

63. The detergent composition of any one of paragraphs 1 to 62, wherein the xanthan lyase variant has an alteration (e.g. a substitution, deletion or insertion, preferably a substitution) in the positions selected from the group consisting of positions: 190, 229, 234, 440, 582, 624, 631, 635, 672, 703, 738, 752, 753, 754, 757, 769, 775, 801, 875, 892, and any combination thereof, preferably 229+672+752+753+769+775+801+875+892;229+672+753+754+769+775+801+875+892; 229+672+752+753+754+769+775+801+875+892; 190+229+234+624+672+753+754+769+775+801+875; 190+229+631+672+703+752+753+769+775+801+875; 190+229+234+582+672+753+754+757+769+775+801+875+892; 229+440+582+624+635+672+738+753+754+757+769+775+801+875+892; or 100+229+360+458+582+672+753+754+757+769+775+801+843+875+892+1008 of SEQ ID NO:6.

64. The detergent composition of any one of paragraphs 1 to 63, wherein the xanthan lyase variant has an alteration (e.g. a substitution, deletion or insertion, preferably a substitution) selected from the group consisting of the following alterations: E229N+N672D+P752K+G753E+A769D+L775A+D801G+K875T+N892Y; E2295+N672D+G753E+5754E+A769D+L775A+D801G+K875T+N892Y; E2295+N672D+P752R+G753E+5754E+A769D+L775A+D801G+K875T+N892Y; A190Q+E229S+1234V+A624E+N672D+G753E+S754E+A769D+L775A+D801G+K875T; A190Q+E229S+T631N+N672D+1703L+P752K+G753E+A769D+L775A+D801G+K875T; A190Q+E229S+1234V+S582K+N672D+G753E+S754E+S757D+A769D+L775A+D801G+K875T+N89 2Y; E229S+N440K+S582K+A624E+S635E+N672D+G738L+G753E+S754E+S757D+A769D+L775A+D80 1G+K875T+N892Y; or S100D+E229S+K360G+D458S+S582K+N672D+G753E+S754E+S757D+A769D+L775A+D801G+A8 43P+K875T+N892Y+N1008D of SEQ ID NO:6.

65. The detergent composition of any one of paragraphs 1 to 64, comprising an endoglucanase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% sequence identity to SEQ ID NO:2 and having an alteration (e.g. a substitution, deletion or insertion, preferably a substitution) in the positions selected from the group consisting of positions: 559+579+697; 512+559+579+697; 18+71+186+408+579+602+651+688+756; 18+189+408+559+579+688+697+756+921+934; 313+488; 880+905+921+934; 302+313+408+579+602+651+697+880+921+934; or 216+313+408+476+579+602+638+651+697+719+880+887+921+934 of SEQ ID NO:2, preferably having an alteration selected from the group consisting of the following alterations: A559N+Y579W+T697G; K512P+A559N+Y579W+T697G; N18G+A71E+A186P+E408D+Y579W+I602T+A651P+A688G+V756Y; N18G+N189K+E408D+A559N+Y579W+A688G+T697G+V756Y+K921R+Y934G; S313D+E408D; R880K+N905D+K921R+Y934G; I302D+S313D+E408D+Y579W+I602T+A651P+T697G+R880K+K921R+Y934G; and N216Q+5313D+E408D+D476R+Y579W+I602T+F638N+A651P+T697G+W719R+R880K+T887K+K9 21R+Y934G of SEQ ID NO:2, the endoglucanase variant preferably having besides the aforementioned alterations no further alterations relative to the parent enzyme of SEQ ID NO:2.

66. The detergent composition of any one of paragraphs 1 to 65, comprising a xanthan lyase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% sequence identity to SEQ ID NO:6 and having an alteration (e.g. a substitution, deletion or insertion, preferably a substitution) in the positions selected from the group consisting of positions: 229+672+752+753+769+775+801+875+892; 229+672+753+754+769+775+801+875+892;229+672+752+753+754+769+775+801+875+892; 190+229+234+624+672+753+754+769+775+801+875; 190+229+631+672+703+752+753+769+775+801+875; 190+229+234+582+672+753+754+757+769+775+801+875+892; 229+440+582+624+635+672+738+753+754+757+769+775+801+875+892; or 100+229+360+458+582+672+753+754+757+769+775+801+843+875+892+1008 of SEQ ID NO:6, preferably having an alteration selected from the group consisting of the following alterations: E229N+N672D+P752K+G753E+A769D+L775A+D801G+K875T+N892Y; E2295+N672D+G753E+S754E+A769D+L775A+D801G+K875T+N892Y; E2295+N672D+P752R+G753E+S754E+A769D+L775A+D801G+K875T+N892Y; A190Q+E229S+I234V+A624E+N672D+G753E+S754E+A769D+L775A+D801G+K875T; A190Q+E229S+T631N+N672D+1703L+P752K+G753E+A769D+L775A+D801G+K875T; A190Q+E229S+I234V+S582K+N672D+G753E+S754E+S757D+A769D+L775A+D801G+K875T+N89 2Y; E229S+N440K+S582K+A624E+S635E+N672D+G738L+G753E+S754E+S757D+A769D+L775A+D80 1G+K875T+N892Y; or S100D+E229S+K360G+D458S+S582K+N672D+G753E+S754E+S757D+A769D+L775A+D801G+A8 43P+K875T+N892Y+N1008D of SEQ ID NO:6, the xanthan lyase variant preferably having besides the afore-mentioned alterations no further alterations relative to the parent enzyme of SEQ ID NO:6.

67. The detergent composition of any one of paragraphs 1 to 66, comprising

(A) an endoglucanase variant selected from those having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% sequence identity to SEQ ID NO:2 and having an alteration (e.g. a substitution, deletion or insertion, preferably a substitution) in the positions selected from the group consisting of positions: 559+579+697; 512+559+579+697; 18+71+186+408+579+602+651+688+756; 18+189+408+559+579+688+697+756+921+934; 313+488; 880+905+921+934; 302+313+408+579+602+651+697+880+921+934; or 216+313+408+476+579+602+638+651+697+719+880+887+921+934 of SEQ ID NO:2, preferably having an alteration selected from the group consisting of the following alterations: (A1) A559N+Y579W+T697G; (A2) K512P+A559N+Y579W+T697G; (A3) N18G+A71E+A186P+E408D+Y579W+I602T+A651P+A688G+V756Y; (A4) N18G+N189K+E408D+A559N+Y579W+A688G+T697G+V756Y+K921R+Y934G; (A5) S313D+E408D; (A6) R880K+N905D+K921R+Y934G; (A7) I302D+S313D+E408D+Y579W+I602T+A651P+T697G+R880K+K921R+Y934G; and (A8) N216Q+S313D+E408D+D476R+Y579W+I602T+F638N+A651P+T697G+W719R+R880K+T887K+K9 21R+Y934G of SEQ ID NO:2; and

(B) a xanthan lyase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% sequence identity to SEQ ID NO:6 and having an alteration (e.g. a substitution, deletion or insertion, preferably a substitution) in the positions selected from the group consisting of positions: 229+672+752+753+769+775+801+875+892; 229+672+753+754+769+775+801+875+892;229+672+752+753+754+769+775+801+875+892; 190+229+234+624+672+753+754+769+775+801+875; 190+229+631+672+703+752+753+769+775+801+875; 190+229+234+582+672+753+754+757+769+775+801+875+892; 229+440+582+624+635+672+738+753+754+757+769+775+801+875+892; or 100+229+360+458+582+672+753+754+757+769+775+801+843+875+892+1008 of SEQ ID NO:6, preferably having an alteration selected from the group consisting of the following alterations: (B1) E229N+N672D+P752K+G753E+A769D+L775A+D801G+K875T+N892Y; (B2) E229S+N672D+G753E+S754E+A769D+L775A+D801G+K875T+N892Y; (B3) E229S+N672D+P752R+G753E+S754E+A769D+L775A+D801G+K875T+N892Y; (B4) A190Q+E229S+I234V+A624E+N672D+G753E+S754E+A769D+L775A+D801G+K875T; (B5) A190Q+E229S+T631N+N672D+1703L+P752K+G753E+A769D+L775A+D801G+K875T; (B6) A190Q+E229S+I234V+S582K+N672D+G753E+S754E+S757D+A769D+L775A+D801G+K875T+N89 2Y; (B7) E229S+N440K+S582K+A624E+S635E+N672D+G738L+G753E+S754E+S757D+A769D+L775A+D80 1G+K875T+N892Y; and (B8) S100D+E229S+K360G+D458S+S582K+N672D+G753E+S754E+S757D+A769D+L775A+D801G+A8 43P+K875T+N892Y+N1008D of SEQ ID NO:6.

68. The detergent composition of paragraph 67, wherein the endoglucanase and/or the xanthan lyase variant, preferably both, do not comprise any further substitution besides those listed in paragraph 67.

69. The detergent composition of any one of paragraphs 1 to 68, comprising the endoglucanase variant Al and any one of the xanthan lyase variants B1, B2, B3, B4, B5, B6, B7 and B8, wherein the respective enzymes preferably have at least 90%, preferably at least 91%, 92%, 93%, 94%, 95%, 96% or 97% sequence identity to SEQ ID NO:2 and SEQ ID NO:6, respectively, more preferably are identical to their respective parent sequence with the exception of the substitutions explicitly listed.

70. The detergent composition of any one of paragraphs 1 to 68, comprising the endoglucanase variant A2 and any one of the xanthan lyase variants B1, B2, B3, B4, B5, B6, B7 and B8, wherein the respective enzymes preferably have at least 90%, preferably at least 91%, 92%, 93%, 94%, 95%, 96% or 97% sequence identity to SEQ ID NO:2 and SEQ ID NO:6, respectively, more preferably are identical to their respective parent sequence with the exception of the substitutions explicitly listed.

71. The detergent composition of any one of paragraphs 1 to 68, comprising the endoglucanase variant A3 and any one of the xanthan lyase variants B1, B2, B3, B4, B5, B6, B7 and B8, wherein the respective enzymes preferably have at least 90%, preferably at least 91%, 92%, 93%, 94%, 95%, 96% or 97% sequence identity to SEQ ID NO:2 and SEQ ID NO:6, respectively, more preferably are identical to their respective parent sequence with the exception of the substitutions explicitly listed.

72. The detergent composition of any one of paragraphs 1 to 68, comprising the endoglucanase variant A4 and any one of the xanthan lyase variants B1, B2, B3, B4, B5, B6, B7 and B8, wherein the respective enzymes preferably have at least 90%, preferably at least 91%, 92%, 93%, 94%, 95%, 96% or 97% sequence identity to SEQ ID NO:2 and SEQ ID NO:6, respectively, more preferably are identical to their respective parent sequence with the exception of the substitutions explicitly listed.

73. The detergent composition of any one of paragraphs 1 to 68, comprising the endoglucanase variant A5 and any one of the xanthan lyase variants B1, B2, B3, B4, B5, B6, B7 and B8, wherein the respective enzymes preferably have at least 90%, preferably at least 91%, 92%, 93%, 94%, 95%, 96% or 97% sequence identity to SEQ ID NO:2 and SEQ ID NO:6, respectively, more preferably are identical to their respective parent sequence with the exception of the substitutions explicitly listed.

74. The detergent composition of any one of paragraphs 1 to 68, comprising the endoglucanase variant A6 and any one of the xanthan lyase variants B1, B2, B3, B4, B5, B6, B7 and B8, wherein the respective enzymes preferably have at least 90%, preferably at least 91%, 92%, 93%, 94%, 95%, 96% or 97% sequence identity to SEQ ID NO:2 and SEQ ID NO:6, respectively, more preferably are identical to their respective parent sequence with the exception of the substitutions explicitly listed.

75. The detergent composition of any one of paragraphs 1 to 68, comprising the endoglucanase variant A7 and any one of the xanthan lyase variants B1, B2, B3, B4, B5, B6, B7 and B8, wherein the respective enzymes preferably have at least 90%, preferably at least 91%, 92%, 93%, 94%, 95%, 96% or 97% sequence identity to SEQ ID NO:2 and SEQ ID NO:6, respectively, more preferably are identical to their respective parent sequence with the exception of the substitutions explicitly listed.

76. The detergent composition of any one of paragraphs 1 to 68, comprising the endoglucanase variant A8 and any one of the xanthan lyase variants B1, B2, B3, B4, B5, B6, B7 and B8, wherein the respective enzymes preferably have at least 90%, preferably at least 91%, 92%, 93%, 94%, 95%, 96% or 97% sequence identity to SEQ ID NO:2 and SEQ ID NO:6, respectively, more preferably are identical to their respective parent sequence with the exception of the substitutions explicitly listed.

77. The detergent composition of paragraph 67 or 68, comprising (1) the endoglucanase variant A1 and the xanthan lyase variant B1; (2) the endoglucanase variant A1 and the xanthan lyase variant B2; (3) the endoglucanase variant A1 and the xanthan lyase variant B3; (4) the endoglucanase variant A1 and the xanthan lyase variant B4; (5) the endoglucanase variant A1 and the xanthan lyase variant B5; (6) the endoglucanase variant A1 and the xanthan lyase variant B6; (7) the endoglucanase variant A1 and the xanthan lyase variant B7; (8) the endoglucanase variant A1 and the xanthan lyase variant B8; (9) the endoglucanase variant A2 and the xanthan lyase variant B1; (10) the endoglucanase variant A2 and the xanthan lyase variant B2; (11) the endoglucanase variant A2 and the xanthan lyase variant B3; (12) the endoglucanase variant A2 and the xanthan lyase variant B4; (13) the endoglucanase variant A2 and the xanthan lyase variant B5; (14) the endoglucanase variant A2 and the xanthan lyase variant B6; (15) the endoglucanase variant A2 and the xanthan lyase variant B7; (16) the endoglucanase variant A2 and the xanthan lyase variant B8; (17) the endoglucanase variant A3 and the xanthan lyase variant Bl; (18) the endoglucanase variant A3 and the xanthan lyase variant B2; (19) the endoglucanase variant A3 and the xanthan lyase variant B3; (20) the endoglucanase variant A3 and the xanthan lyase variant B4; (21) the endoglucanase variant A3 and the xanthan lyase variant B5; (22) the endoglucanase variant A3 and the xanthan lyase variant B6; (23) the endoglucanase variant A3 and the xanthan lyase variant B7; (24) the endoglucanase variant A3 and the xanthan lyase variant B8; (25) the endoglucanase variant A4 and the xanthan lyase variant Bl; (26) the endoglucanase variant A4 and the xanthan lyase variant B2; (27) the endoglucanase variant A4 and the xanthan lyase variant B3; (28) the endoglucanase variant A4 and the xanthan lyase variant B4; (29) the endoglucanase variant A4 and the xanthan lyase variant B5; (30) the endoglucanase variant A4 and the xanthan lyase variant B6; (31) the endoglucanase variant A4 and the xanthan lyase variant B7; (32) the endoglucanase variant A4 and the xanthan lyase variant B8; (33) the endoglucanase variant A5 and the xanthan lyase variant B1; (34) the endoglucanase variant A5 and the xanthan lyase variant B2; (35) the endoglucanase variant A5 and the xanthan lyase variant B3; (36) the endoglucanase variant A5 and the xanthan lyase variant B4; (37) the endoglucanase variant A5 and the xanthan lyase variant B5; (38) the endoglucanase variant A5 and the xanthan lyase variant B6; (39) the endoglucanase variant A5 and the xanthan lyase variant B7; (40) the endoglucanase variant A5 and the xanthan lyase variant B8; (41) the endoglucanase variant A6 and the xanthan lyase variant B1; (42) the endoglucanase variant A6 and the xanthan lyase variant B2; (43) the endoglucanase variant A6 and the xanthan lyase variant B3; (44) the endoglucanase variant A6 and the xanthan lyase variant B4; (45) the endoglucanase variant A6 and the xanthan lyase variant B5; (46) the endoglucanase variant A6 and the xanthan lyase variant B6; (47) the endoglucanase variant A6 and the xanthan lyase variant B7; (48) the endoglucanase variant A6 and the xanthan lyase variant B8; (49) the endoglucanase variant A7 and the xanthan lyase variant B1; (50) the endoglucanase variant A7 and the xanthan lyase variant B2; (51) the endoglucanase variant A7 and the xanthan lyase variant B3; (52) the endoglucanase variant A7 and the xanthan lyase variant B4; (53) the endoglucanase variant A7 and the xanthan lyase variant B5; (54) the endoglucanase variant A7 and the xanthan lyase variant B6; (55) the endoglucanase variant A7 and the xanthan lyase variant B7; (56) the endoglucanase variant A7 and the xanthan lyase variant B8; (57) the endoglucanase variant A8 and the xanthan lyase variant B1; (58) the endoglucanase variant A8 and the xanthan lyase variant B2; (59) the endoglucanase variant A8 and the xanthan lyase variant B3; (60) the endoglucanase variant A8 and the xanthan lyase variant B4; (61) the endoglucanase variant A8 and the xanthan lyase variant B5; (62) the endoglucanase variant A8 and the xanthan lyase variant B6; (63) the endoglucanase variant A8 and the xanthan lyase variant B7; (64) the endoglucanase variant A8 and the xanthan lyase variant B8; or (65) the endoglucanase variant and xanthan lyase variant combinations disclosed in Talbes 34-36.

78. The detergent composition of any of paragraphs 1-77, further comprising one or more detergent components.

79. The detergent composition of any of paragraphs 1-78, further comprising one or more additional enzymes selected from the group consisting of: endoglucanases, proteases, amylases, lichenases, lipases, cutinases, cellulases, xanthan lyases, xyloglucanases, pectinases, pectin lyases, xanthanases, peroxidases, haloperoxygenases, catalases and mannanases, or any mixture thereof.

80. The detergent composition of any of paragraphs 1-79, wherein said composition is in form of a bar, a homogenous tablet, a tablet having two or more layers, a pouch having one or more compartments, a regular or compact powder, a granule, a paste, a gel, or a regular, compact or concentrated liquid.

81. Use of a detergent composition of any of paragraphs 1-80, wherein said use is for degrading xanthan gum.

82. The use of paragraph 81, wherein said endoglucanase variant and/or xanthan lyase variant has an enzyme detergency benefit.

83. A method for degrading xanthan gum comprising: applying a detergent composition of any of paragraphs 1-80 to a xanthan gum.

84. The method of paragraph 83, wherein said xanthan gum is on a surface or hard surface.

The present invention is further described by the following examples that should not be construed as limiting the scope of the invention.

### EXAMPLES

### Example 1

### Construction of GH9 Endoglucanase Variants of the Mature Parent Eendoglucanase Having SEQ ID NO:2

A linear integration vector-system was used for cloning of the mature parent nucleotide sequence having SEQ ID NO:1 (same also disclosed as mature peptide within SEQ ID NO:1 of WO2013/167581) coding for the mature parent polypeptide of the GH9 endoglucanase of SEQ ID NO:2, and its variants. The linear integration construct was a PCR fusion made by fusing the gene between two Bacillus subtilis homologous chromosomal regions along with strong promoters and a chloramphenicol resistance marker. The fusion was made by Splicing by Overlap Extension (SOE) PCR (Horton et al. (1989) Engineering hybrid genes without the use of restriction enzymes, or gene splicing were produced by overlap extension (Gene 77: 61-68). The SOE PCR method is also described in patent application WO2003/095658. The gene was expressed under the control of a triple promoter system (as described in WO99/43835), consisting of the promoters from Bacillus licheniformis alpha-amylase gene (amyL), Bacillus amyloliquefaciens alpha-amylase gene (amyQ), and the Bacillus thuringiensis cryIIIA promoter including stabilizing sequence. The gene coding for chloramphenicol acetyltransferase was used as marker (described in e.g. Diderichsen et al. (1993) A useful cloning vector for Bacillus subtilis. Plasmid 30:312). The final gene constructs were integrated on the Bacillus chromosome by homologous recombination into the pectate lyase locus. The gene fragments were amplified from chromosomal DNA of the corresponding strains with gene specific primers containing overhang to the two flanking vector fragments. All genes were expressed with a Bacillus licheniformis alpha-amylase secretion signal having the nucleotide sequence of SEQ ID NO:3 and the amino acid sequence of SEQ ID NO:4 replacing the native secretion signal.

Variants of the mature parent GH9 endocluconase from Paenibacillus sp-62047 having SEQ ID NO:2 as described in Examples 3-4 below were made by the megaprimer mutagenesis method using specifically designed mutagenic oligonucleotides introducing desired mutations in the resulting sequence. Design and production methods for such mutagenic oligonucleotides introducing desired mutations into target sequences are well known to those skilled in the art. Consequently, mutagenic oligos were designed and synthesized corresponding to the DNA sequence flanking the desired site(s) of mutation, separated by the DNA base pairs defining the substitutions. The final expression cassette composed the reference GH9 endocluconase from Paenibacillus sp-62047 as described above (i.e. parent GH9 endocluconase having SEQ ID NO:1). Sucessful introduction of the desired substitutions was confirmed by DNA sequencing of the GH9 endogluconase gene. An aliquot of the PCR product was subsequently transformed into Bacillus subtilis. Transformants were selected on LB agar plates supplemented with 10 mM K₂PO₄, 0.4% extra glucose and 6 μg of chloramphenicol per mL. The resulting recombinant Bacillus subtilis clone containing the integrated expression construct was grown in liquid culture as described below. The enzyme containing supernatants were harvested and the enzymes (variants) were either stress tested using a reducing sugar assay or purified as described below.

Variants above were produced by fermentation using standard protocols (TB-glycerol media containing a standard trace metal mix as described in F. William Studier (2005) Protein production by auto-induction in high-density shaking cultures, Protein Expression and Purification, 41: 207-234) and grown for 4 days at 30° C. before harvested). Supernatants of samples used for stress testing were inoculated from an overnight culture grown at 37° C. and subsequently fermented in 96-well plate format (TB-glycerol media described above without calcium in the trace metal mix for 4 days 30° C.).

### Example 2

### Purification of GH9 Endoglucanase Variants

The culture broth was centrifuged at 13'000 rpm (45 min, 18° C., F125-6×500 rotor) using a Sorval RC-6 plus centrifuge (ThermoFisher Scientific). The supernatant was supplemented with (NH₄)₂SO₄ to a final concentration of 0.8 M. The mixture was filtered using 0.2 μm bottle-top rapid flow filters (Nalgene). The mixture was loaded on a 50 mL Phenyl Sepharose High Performance (GE Healthcare, Uppsala, Sweden) pre-equilibrated with 20 mM Tris-HCl, pH 8.0 with 0.8 M (NH₄)₂SO₄. Flowrate was set to 3 mL/min. After protein loading, the flow rate was increased to 5 mL/min and unbound or loosely bound protein was washed out by several column volumes of equilibration buffer. Elution was carried out by step-wise increase of elution buffer (20 mM Tris-HCl, pH 8.0). The target protein eluted during the (75-100%) elution step. Fractions of 8 mL were collected during the purification. The fractions were evaluated using SDS-PAGE (NuPAGE, Invitrogen). Fractions eluting with 20 mM Tris-HCl, pH 8.0 were pooled and desalted on a 350 mL G25 desalting column pre-equilibrated with 20 mM Tris-HCl, pH 8.5. The desalted protein solution was applied on a 20 mL Source15Q column pre-equilibrated with 20 mM Tris-HCl, pH 8.5 at 2 mL/min. Unbound or loosely bound proteins were washed using at least two column volumes of equilibration buffer until a stable UV baseline was obtained. The flow rate was raised to 4 mL/min and elution was done by a linear NaCl gradient using the elution buffer (20 mM Tris-HCl, pH 8.5 +750 mM NaCl). 3 mL fractions were collected during the purification. SDS-PAGE was used to evaluate the fractions. Pure fractions were pooled and concentrated if necessary using Vivaspin 20 (10 kDa Cut-off, Sartorius). Protein concentration was determined using absorbance measurements at 280 nm.

### Example 3

### Detergent Stability Assay

GH9 endoglucanase (EG) activity (EC 3.2.1.4) was determined by reducing ends on xanthan gum pre-treated with xanthan lyase using the colorimetric assay developed by Lever (1972), Anal. Biochem. 47: 273-279, 1972. Pre-treated xanthan gum is a modified form of the xanthan sugar, where the terminal pyruvated mannose from side chains is removed (prepared according to Nankai et al. (1999) from the source Keltran). The GH9 mature parent endoglucanase and its variants cleave at beta-(1,4)-glucosyl bonds in the glucan backbone of pretreated xanthan gum releasing glucans with a reducing end which can be determined by reaction with p-Hydroxybenzoic acid hydrazide (PAHBAH). The increase of colour is proportional to the enzyme activity under the conditions used in the assay (e.g. Table 1) and used to estimate the residual activity (RA), half-life (T1/2) and the half-life improvement factor (HIF).

TABLE 1
Description of assays
Stress assay:

| | |
|---|---|
| Detergent | Persil Universal Gel |
| Assay buffer (AB) | 50 mM MOPS, 4 mM CaCl₂, 0.01% Triton X-100, pH 7.0 |
| Reference sample conditions | 4° C. for 5-138 h |
| Stress conditions | 25, 26, 28 or 30° C. for 5-138 h |

Activity assay:

| | |
|---|---|
| Substrate concentration | 4 mg/mL modified xanthan gum |
| Xanthan gum incubation | 50° C. for 1 h |
| PAHBAH solution | 15 mg/mL 4-hydroxybenzoic acid hydrazide (PAHBAH), |
| | 50 g/L potassium sodium tartrate tetrahydrate, 20 g/L |
| | NaOH |
| PAHBAH development | 95° C. for 10 min |

Method Steps:

30 μL enzyme sample (purified variant, 10-150 ppm) was mixed with 270 μL detergent using magnetic stirring for 15 minutes in a micro titer plate (MTP). This plate was designated as the “stress MTP”. 20 μL of the mixture was transferred to a new MTP and diluted 100-fold using a 2-step dilution (2×10-fold dilution). The sample was diluted into assay buffer (AB): 50 mM MOPS, 4 mM CaCl₂, 0.01% Triton X-100, pH 7.0. This diluted MTP is the “reference MTP” and is stored at 4° C. for 5-138 h (ata time interval equal to that of the stress MTP below). The stress MTP was incubated at 25, 26, 28 or 30° C. for 5-138 h. After incubation, the stress MTP was initially mixed by magnetic stirring for 15 min, and the stress MTP was then diluted 100-fold as described for the reference MTP. To assess the enzymatic activity, 50 μL of diluted enzyme:detergent sample (from both reference and stress MTPs) was mixed with 50 μL 4 mg/mL modified xanthan gum in PCR plates. The samples were then incubated at 50° C. for 1 h. Finally, the level of reducing ends was estimated by adding 75 μL PAHBAH solution (15 mg/mL PAHBAH, 50 g/L potassium sodium tartrate tetrahydrate, 20 g/L NaOH) to all samples in the PCR plates. The samples were then incubated at 95° C. for 10 min. After cooling down to room temperature, the absorbance at 405 nm was measured.The residual activity (RA) was calculated using the following formula:

RA (%)=(Abs(Stress))/(Abs(Ref))×100%

Abs(Stress): The absorbance at 405 nm of the sample in the stress MTP (incubated at 25, 26, 28 or 30° C.) after subtracting relevant background absorbance contributions.

Abs(Ref): The absorbance at 405 nm of the sample in the reference MTP (incubated at 4° C.).
Also, the half-lives for the degradation of each variant and parent endoglucanase were
calculated using the following formula (by applying 1st order kinetics for the degradation

of EG):

$$T 1 / 2 = - \frac{ln ( 2 ) \times T}{ln ( \frac{Abs ( Stress )}{Abs ( Ref )} )}$$

T: The incubation time. Abs(Stress) and Abs(Ref): See above.

Half-life improvement factors (HIFs) can then be calculated as:

$$H I F = \frac{T 1 / 2 , variant}{T 1 / 2 , w t}$$

T½, variant: The half-life for a specific variant
T½, wt (or wild-type): The half-life for EG wt (EG wild type), wherein said T½ wt is
T½ of the mature parent endoglucanase with SEQ ID NO:2.

The HIFs of the tested variants are shown in Tables 2-7 below: All half-life values of the variants measured in culture supernatant were calculated relative to the half-life of GH9 wild-type (mature parent endoclucanase with SEQ ID NO:2) measured as culture supernatant. All half-life values of the variants measured as purified protein were calculated relative to the half-life of GH9 wild-type (mature parent endoclucanase with SEQ ID NO:2) measured as purified protein. HIFs of all variants were calculated based on the wild-type endoglucanase (SEQ ID NO:2) incubated at the same detergent concentration and temperature (HIF of wild-type=1 in all tables, per definition).

TABLE 2
Variants of the mature parent GH9 endoglucanase
(SEQ ID NO: 2) with corresponding half-life improvement
factors (HIF) measured as culture supernatants

| Regions (as defined herein) | Alteration | HIF |
|---|---|---|
| 4 | N285G | 1.4 |
| 5 | W333L | 1.2 |
| 5 | T353D | 1.8 |
| 6 | N558F | 1.4 |
| 7 | T633V | 1.1 |
| 7 | D635L | 2.4 |
| 7 | D635M | 1.2 |
| 7 | D635T | 1.1 |
| 7 | F638Y | 1.1 |
| 7 | T639D | 1.2 |
| 9 | G994N | 3.7 |

TABLE 3
Variants of the mature parent GH9 endoglucanase (SEQ ID
NO: 2) with corresponding half-life improvement factors (HIF)
measured as culture supernatants

| Regions (as defined herein) | Alteration | HIF |
|---|---|---|
| 4 | K281T | 1.3 |
| 6 | N558NP | >10.0 |
| 6 | G563E | 1.14 |
| 6 | I575M | >10.0 |
| 6 | I575A | 1.3 |
| 9 | K921D | 1.4 |

TABLE 4
Variants of the mature parent GH9 endoglucanase (SEQ
ID NO: 2) with corresponding half-life improvement factors (HIF)
measured as culture supernatants

| Regions (as defined herein) | Alteration | HIF |
|---|---|---|
| 2 | I125V | 1.1 |
| 2 | A126R | 1.1 |
| 2 | K130R | 1.1 |
| 3 | K213R | 1.2 |
| 3 | A221R | 1.1 |
| 3 | K228E | 1.1 |
| 3 | K228I | 1.2 |
| 3 | G230F | 1.2 |
| 3 | G230L | 1.2 |
| 3 | G230A | 1.2 |
| 3 | G230H | 1.2 |
| 3 | G230N | 1.1 |
| 3 | G230W | 1.1 |
| 3 | G230T | 1.2 |
| 3 | F231Y | 1.2 |
| 3 | F231N | 1.1 |
| 3 | V232R | 1.1 |
| 3 | V232G | 1.1 |
| 3 | H235D | 1.1 |
| 3 | N240Q | 1.2 |
| 3 | G243K | 1.1 |
| 3 | G243R | 1.2 |
| 3 | A249N | 1.1 |
| 4 | A278S | 1.1 |
| 4 | K281F | 1.1 |
| 4 | K281V | 1.2 |
| 4 | K281Y | 1.1 |
| 4 | K281H | 1.1 |
| 4 | K281Q | 1.1 |
| 4 | K281N | 1.2 |
| 4 | K281W | 1.1 |
| 4 | N285L | 1.9 |
| 4 | N285M | 1.8 |
| 4 | N285S | 1.2 |
| 4 | N285P | 1.6 |
| 4 | N285T | 1.1 |
| 4 | N285Y | 1.8 |
| 4 | N285H | 2.4 |
| 4 | N285K | 1.4 |
| 4 | N285D | 1.6 |
| 4 | N285W | 1.3 |
| 4 | N285R | 1.5 |
| 4 | T292F | 1.2 |
| 4 | T292L | 2 |
| 4 | T292I | 1.5 |
| 4 | T292V | 1.5 |
| 4 | T292S | 1.3 |
| 4 | T292P | 1.6 |
| 4 | T292Y | 1.2 |
| 4 | T292Q | 1.4 |
| 4 | T292N | 1.1 |
| 4 | T292K | 1.2 |
| 4 | T292D | 1.3 |
| 4 | T292G | 1.4 |
| 4 | F297L | 1.1 |
| 5 | A346H | 1.1 |
| 6 | G556S | 1.2 |
| 6 | N558D | 1.4 |
| 6 | N558M | 1.2 |
| 6 | N558Q | 1.3 |
| 6 | N558I | 1.2 |
| 6 | N558Y | 1.1 |
| 6 | N558H | 1.1 |
| 6 | A559N | 1.7 |
| 6 | A559F | 1.2 |
| 6 | A559M | >10.0 |
| 6 | A559P | 1.5 |
| 6 | A559Y | 1.3 |
| 6 | A559H | 1.6 |
| 6 | A559Q | 1.4 |
| 6 | A559D | 1.5 |
| 6 | A559R | 1.1 |
| 6 | A559G | 1.1 |
| 6 | A559I | 1.1 |
| 6 | A559S | 1.2 |
| 6 | S560P | 1.3 |
| 6 | S560K | 1.2 |
| 6 | S560G | 1.4 |
| 6 | S560D | 1.3 |
| 6 | T561P | 1.6 |
| 6 | T561E | 1.1 |
| 6 | T561Q | 1.1 |
| 6 | T561S | 1.1 |
| 6 | T561D | 1.2 |
| 6 | A564I | 1.5 |
| 6 | A564Y | 1.2 |
| 6 | A564H | 1.1 |
| 6 | A564Q | 1.2 |
| 6 | A564K | 1.5 |
| 6 | A564E | 1.4 |
| 6 | E565M | 1.2 |
| 6 | V567F | 1.1 |
| 6 | K568R | 1.3 |
| 6 | L569F | 1.2 |
| 6 | L569Y | 1.2 |
| 6 | L569D | 1.2 |
| 6 | L569E | 1.3 |
| 6 | P570F | 1.3 |
| 6 | P570L | 1.6 |
| 6 | P570I | 1.8 |
| 6 | P570M | 2.1 |
| 6 | P570V | 2.6 |
| 6 | P570S | 4.9 |
| 6 | P570T | 5.4 |
| 6 | P570A | 4.4 |
| 6 | P570Y | 2.1 |
| 6 | P570H | 2.6 |
| 6 | P570Q | 5.1 |
| 6 | P570N | 3.3 |
| 6 | P570K | 4.2 |
| 6 | P570E | 1.6 |
| 6 | P570W | 1.7 |
| 6 | P570R | 3.6 |
| 6 | P570G | 1.4 |
| 6 | I575D | 1.5 |
| 6 | I575E | 3.7 |
| 6 | I576F | 1.8 |
| 6 | I576M | 1.2 |
| 6 | I576P | 3.3 |
| 6 | D578R | 1.1 |
| 6 | Y579F | 1.1 |
| 6 | Y579W | 2.1 |
| 6 | V580L | 1.2 |
| 6 | D583M | 1.1 |
| 6 | Q589G | 1.1 |
| 6 | P590S | 1.1 |
| 6 | P590T | 1.1 |
| 6 | P590E | 1.2 |
| 6 | E591L | 1.5 |
| 6 | G592D | 1.1 |
| 6 | S593P | 1.4 |
| 6 | S593H | 1.3 |
| 6 | S593Q | 1.3 |
| 6 | S593N | 1.5 |
| 6 | S593K | 1.2 |
| 6 | S593D | 1.3 |
| 6 | S593E | 1.3 |
| 6 | S593R | 1.3 |
| 7 | S616D | 1.2 |
| 7 | K627L | 1.8 |
| 7 | K627M | 2.2 |
| 7 | K627V | 2.4 |
| 7 | K627S | 1.1 |
| 7 | K627T | 1.7 |
| 7 | K627Q | 2.5 |
| 7 | K627R | 4.3 |
| 7 | I630F | 1.4 |
| 7 | I630V | 1.2 |
| 7 | I630Y | 1.2 |
| 7 | D635A | 1.3 |
| 7 | D635P | 1.1 |
| 7 | D635N | 1.3 |
| 7 | D635K | 1.3 |
| 7 | D635E | 1.4 |
| 7 | D635G | 1.1 |
| 7 | D635W | 1.1 |
| 7 | S636L | 1.6 |
| 7 | S636M | 1.9 |
| 7 | S636A | 1.4 |
| 7 | S636H | 1.4 |
| 7 | S636Q | 2.4 |
| 7 | S636N | 2 |
| 7 | S636K | 1.8 |
| 7 | S636R | 1.3 |
| 7 | F638I | 1.7 |
| 7 | F638V | 1.4 |
| 7 | F638T | 1.2 |
| 7 | F638L | 1.1 |
| 7 | F638H | 1.3 |
| 7 | T639V | 1.1 |
| 7 | T639S | 1.1 |
| 7 | T639L | 1.2 |
| 7 | T639I | 2.1 |
| 7 | T639M | 1.2 |
| 7 | T639A | 2.2 |
| 7 | T639E | 1.8 |
| 7 | T639W | 1.3 |
| 7 | T639G | 2.1 |
| 7 | Y641E | 1.2 |
| 7 | S642T | 2.1 |
| 7 | S642N | 1.3 |
| 7 | N643D | 2.0 |
| 7 | N643H | 2.1 |
| 7 | N643T | 1.2 |
| 7 | T644F | 1.1 |
| 7 | A651P | 1.5 |
| 8 | S810R | 1.2 |
| 8 | A811S | 1.1 |
| 8 | V812F | 1.3 |
| 8 | V812I | 1.2 |
| 8 | V812M | 1.3 |
| 8 | V812W | 1.2 |
| 8 | V812R | 1.1 |
| 8 | N815V | 1.1 |
| 8 | N815Y | 1.1 |
| 8 | N815E | 1.2 |
| 8 | N815W | 1.1 |
| 8 | N815R | 1.1 |
| 8 | S823Q | 1.1 |
| 8 | A824T | 1.1 |
| 8 | T825N | 1.1 |
| 8 | T825W | 1.1 |
| 8 | T825A | 1.2 |
| 8 | T825D | 1.1 |
| 8 | V827I | 1.2 |
| 8 | V827M | 1.5 |
| 8 | V827S | 1.2 |
| 9 | T843V | 1.1 |
| 9 | D870F | 1.2 |
| 9 | D870L | 1.3 |
| 9 | D870I | 1.3 |
| 9 | D870M | 1.4 |
| 9 | D870V | 1.5 |
| 9 | D870S | 1.3 |
| 9 | D870T | 1.1 |
| 9 | D870Y | 1.1 |
| 9 | D870H | 1.2 |
| 9 | D870Q | 1.3 |
| 9 | D870N | 1.5 |
| 9 | D870K | 1.4 |
| 9 | D870E | 1.4 |
| 9 | D870W | 1.1 |
| 9 | D870R | 1.2 |
| 9 | D870G | 1.2 |
| 9 | P871F | 1.3 |
| 9 | P871L | 1.3 |
| 9 | P871I | 1.5 |
| 9 | P871M | 1.3 |
| 9 | P871V | 1.3 |
| 9 | P871S | 1.3 |
| 9 | P871T | 1.4 |
| 9 | P871A | 1.3 |
| 9 | P871Y | 1.2 |
| 9 | P871H | 1.3 |
| 9 | P871Q | 1.5 |
| 9 | T872S | 1.1 |
| 9 | T872F | 1.1 |
| 9 | T872A | 1.1 |
| 9 | T872Y | 1.2 |
| 9 | T872H | 1.6 |
| 9 | T872Q | 1.5 |
| 9 | T872N | 1.5 |
| 9 | T872K | 1.4 |
| 9 | T872D | 1.4 |
| 9 | T872E | 1.5 |
| 9 | T872W | 1.5 |
| 9 | T872R | 1.7 |
| 9 | T872G | 1.3 |
| 9 | D873K | 1.1 |
| 9 | D873E | 1.2 |
| 9 | T874V | 1.2 |
| 9 | T874S | 1.3 |
| 9 | T874P | 1.1 |
| 9 | T874A | 1.2 |
| 9 | T874H | 1.1 |
| 9 | T874Q | 1.1 |
| 9 | T874N | 1.2 |
| 9 | T874K | 1.3 |
| 9 | V881Q | 1.2 |
| 9 | T883K | 1.3 |
| 9 | Y884H | 1.8 |
| 9 | A885F | 5.4 |
| 9 | A885Q | 1.3 |
| 9 | A885N | 1.1 |
| 9 | T887L | 1.1 |
| 9 | T887I | 1.1 |
| 9 | T887S | 1.6 |
| 9 | T887H | 1.1 |
| 9 | T887R | 1.1 |
| 9 | K894E | 1.1 |
| 9 | N920D | 1.3 |
| 9 | K921R | 2.4 |
| 9 | K921E | 1.2 |
| 9 | T932A | 1.5 |
| 9 | N933V | 1.4 |
| 9 | N933S | 2.5 |
| 9 | Y934G | 4.8 |
| 9 | Y934M | 1.2 |
| 9 | Y934S | 3.8 |
| 9 | Y934A | 3.7 |
| 9 | Y934Q | 4.2 |
| 9 | Y934N | 2.8 |
| 9 | Y934E | 2.3 |
| 9 | Y934W | 1.5 |
| 9 | Y934R | 6 |
| 9 | T935W | 1.1 |
| 9 | A937F | 1.1 |
| 9 | A937V | 1.7 |
| 9 | A937S | 1.1 |
| 9 | A937T | 1.4 |
| 9 | A937Q | 1.1 |
| 9 | A937D | 1.4 |
| 9 | A937E | 2.6 |
| 9 | V938I | 1.8 |
| 9 | K939I | 1.8 |
| 9 | K939V | 3.4 |
| 9 | D940E | 1.1 |
| 9 | N941S | 2.4 |
| 9 | N941H | 1.6 |
| 9 | N941D | 1.3 |
| 9 | A942P | 1.7 |
| 9 | A942E | 1.2 |
| 9 | D943Y | 1.1 |
| 9 | D943H | 1.2 |
| 9 | R950V | 1.2 |
| 9 | R950H | 1.2 |
| 9 | R950N | 1.4 |
| 9 | F952S | 1.2 |
| 9 | F952W | 1.1 |
| 9 | N953Y | 1.7 |
| 9 | G954L | 1.4 |
| 9 | Y960F | 1.4 |
| 9 | A964N | 1.2 |
| 9 | A964C | 1.2 |
| 9 | N966P | 1.1 |
| 9 | N966C | 1.5 |
| 9 | G971A | 1.1 |
| 9 | Q974K | 1.1 |
| 9 | Q974C | 1.1 |
| 9 | Q989I | 1.1 |
| 9 | Q991L | 1.1 |
| 9 | Q991I | 1.2 |
| 9 | Q991M | 1.1 |
| 9 | Q991V | 1.2 |
| 9 | Q991T | 1.3 |
| 9 | Q991K | 1.1 |
| 9 | Q991C | 1.1 |
| 9 | S995I | 1.1 |
| 9 | S995V | 1.1 |
| 9 | S995Q | 1.1 |
| 9 | S995R | 1.1 |
| 9 | S995C | 1.3 |
| 9 | G998V | 1.1 |
| 9 | G998A | 1.1 |
| 9 | S1006T | 1.2 |
| 9 | S1006A | 1.1 |
| 9 | S1006K | 1.1 |
| 9 | S1006R | 1.2 |
| 9 | Y1010W | 1.1 |
| 9 | L1011M | 1.2 |
| 9 | L1011S | 1.3 |
| 9 | L1011A | 1.4 |
| 9 | L1011Q | 1.1 |
| 9 | L1011N | 1.1 |
| 9 | L1011D | 1.2 |
| 9 | L1011E | 1.2 |
| 9 | R1029N | 1.2 |
| 9 | F1030M | 1.1 |
| 9 | K1031I | 1.2 |
| 9 | K1031S | 1.4 |
| 9 | K1031T | 2.1 |
| 9 | K1031H | 1.2 |
| 9 | V1032G | 1.3 |
| 9 | K1035A | 1.2 |
| 9 | A1037E | 1.4 |
| 9 | A1037W | 1.6 |
| 9 | S1038L | 1.1 |
| 9 | S1038I | 1.2 |
| 9 | L1040N | 1.3 |
| 9 | L1040E | 1.1 |
| 9 | G1041F | 1.1 |
| 9 | L1044F | 1.1 |
| 9 | L1044S | 1.1 |
| 9 | L1044N | 1.1 |
| 9 | L1044W | 1.1 |
| 9 | P1045Q | 1.2 |
| 9 | P1045W | 1.2 |

TABLE 5
Variants of the mature parent GH9 endoglucanase (SEQ
ID NO: 2) with corresponding half-life improvement factors (HIF)
measured as culture supernatants

| Alteration | HIF |
|---|---|
| A559N + Y579F | 2.8 |
| A564E + Y579F | 1.1 |
| A559N + Y579W | 3.2 |
| G562P + Y579W | 1.4 |
| A564D + Y579W | 2.1 |
| A559N + Y579W + K99R | 3.8 |
| A559N + Y579W + K281R | 3.4 |
| K281R + A559N + Y579W | 3.5 |
| A559N + Y579W + S616D | 5.6 |
| A559N + Y579W + S636N | 9 |
| A559N + Y579W + A651P | 4.2 |
| A559N + Y579W + K948E | 2.6 |
| A559N + Y579W + K1009E | 2.4 |
| A559N + Y579W + K627R | 7.8 |
| Y579W + K921R | 3.2 |
| A559N + Y579W + K921R | 6.1 |
| K99R + Y579W | 1.6 |
| Y579W + A651P | 2.7 |
| Y579W + K948E | 1.4 |
| Y579W + K1009E | 1.5 |
| A559N + Y579W + Y934G | 16.0 |
| A559N + Y579W + K921R + Y934G | 29.5 |
| A559N + Y579W + K627M | 6.4 |
| A559N + Y579W + K627R + S616D | 18.1 |
| A559N + Y579F + K627R | 4.4 |
| A559N + Y579W + K921R + A651P | 9.9 |
| A559N + Y579W + K921R + K627R | 17.6 |
| A559N + Y579W + K921R + S636K | 5.9 |
| A559N + Y579W + K921R + S616D | 4.3 |
| A559N + Y579W + K921R + S636N | 8.9 |
| A559N + Y579W + K921R + K627R + S636N | 26.7 |
| A559N + Y579W + S636N + A651P | 9.6 |
| A559N + Y579W + S616D + A651P | 9.4 |
| A559N + Y579W + S616D + S636K | 6.5 |
| A559N + Y579W + S616D + K921R + Y934G | 19.9 |
| A559N + Y579W + A651P + K627M | 10.6 |
| A559N + Y579W + A651P + S636K | 8.2 |
| A559N + Y579W + A651P + K627R + S636N | 37.0 |
| A559N + Y579W + A651P + S616D | 9.0 |
| A559N + Y579W + A651P + K921R + Y934G | 21.1 |
| S636N + Y934G | 1.6 |
| S636N + K921R | 1.4 |
| S636N + K627R | 2.4 |
| S636N + Y579W | 1.6 |
| F638I + Y934G | 3.0 |
| F638I + K921R | 1.4 |
| F638I + K627R | 2.2 |
| F638I + Y579W | 1.3 |
| K627R + K51Q | 1.3 |
| K627R + K451S | 1.4 |
| K627R + A559N | 1.5 |
| K627R + Y579W | 3.9 |
| Y579W + Y934G | 4.1 |
| A651P + F638I | 1.2 |
| P570Q + A651P | 2.7 |
| P570Q + K921R | 1.6 |
| P570Q + K627R | 5.1 |
| P570Q + A559N | 1.8 |
| P570Q + Y579W | 3.4 |
| P570Q + F638I | 1.2 |
| P570K + Y579W | 2.6 |
| P570K + F638I | 1.5 |
| P570T + A651P | 2.6 |
| P570T + S636N | 3.4 |
| P570T + Y934G | 4.3 |
| P570T + F638I | 1.4 |
| P570T + K921R | 1.5 |
| P570T + K627R | 3.9 |
| P570T + A559N | 1.8 |
| P570T + A885F | 1.2 |
| A885F + Y934G | 1.5 |
| A885F + K627R | 1.2 |
| A559N + Y579W + S636L | 1.3 |
| A559N + Y579W + F638I | 2.1 |
| A559N + Y579W + D870M | 1.1 |
| A559N + Y579W + S560P | 4.1 |
| A559N + Y579W + A564I | 1.3 |
| A559N + Y579W + P570N | 2.8 |
| A559N + Y579W + P570K | 5.2 |
| A559N + Y579W + P570R | 5.4 |
| A559N + Y579W + P570A | 4.1 |
| A559N + Y579W + P570T | 3.8 |
| A559N + Y579W + P570S | 3.9 |
| A559N + Y579W + P570Q | 3.3 |
| A559N + Y579W + P570H | 3.7 |

TABLE 6
Variants of the mature parent GH9 endoglucanase (SEQ ID NO: 2) with
corresponding half-life improvement factors (HIF) measured as
purified samples

| Regions (as defined herein) | Alteration | HIF |
|---|---|---|
| 6 | N558E | 1.1 |
| 6 | A559P | 1.4 |
| 6 | A559N | 1.6 |
| 6 | A559H | 1.5 |
| 6 | T561P | 1.1 |
| 6 | A564E | 1.2 |
| 6 | P570A | 7.0 |
| 6 | P570Q | 7.0 |
| 6 | P570R | 7.0 |
| 6 | P570S | 7.0 |
| 6 | P570K | 6.7 |
| 6 | P570T | 6.3 |
| 6 | P570N | 2.9 |
| 6 | Y579W | 2.5 |
| 6 | Y579F | 1.3 |
| 6 | T581M | 1.2 |
| 7 | S616D | 1.4 |
| 7 | K627R | 2.1 |
| 7 | K627M | 2.1 |
| 7 | K627Q | 1.2 |
| 7 | S636N | 1.8 |
| 7 | S636Q | 1.5 |
| 7 | S636R | 1.4 |
| 7 | S636K | 1.4 |
| 7 | S636M | 1.3 |
| 7 | S636H | 1.1 |
| 7 | F638I | 1.6 |
| 7 | F638L | 1.2 |
| 7 | N643D | 1.5 |
| 7 | A651P | 1.3 |
| 7 | A651S | 1.2 |
| 9 | A885F | 1.1 |
| 9 | A885Q | 1.1 |
| 9 | K921R | 4.2 |
| 9 | Y934R | 14 |
| 9 | Y934G | 6.2 |
| 9 | N966C | 1.3 |
| 9 | L1011A | 1.1 |
| 9 | K1031I | 1.3 |

TABLE 7
Variants of the mature parent GH9 endoglucanase (SEQ ID NO: 2) with
corresponding half-life improvement factors (HIF) measured as
purified samples

| Alteration | HIF |
|---|---|
| N558K + A559K + S560F + T561P + G562W | 3.8 |
| A559N + P570A + Y579W | 4.6 |
| A559N + P570H + Y579W | 3.5 |
| A559N + P570K + Y579W | 10.2 |
| A559N + P570N + Y579W | 3.4 |
| A559N + P570Q + Y579W | 4.5 |
| A559N + P570R + Y579W | 5.5 |
| A559N + P570S + Y579W | 5.0 |
| A559N + P570T + Y579W | 6.0 |
| A559N + S560P + Y579W | 2.9 |
| A559N + Y579W + A651P | 1.6 |
| A559N + Y579W + A651P + Y934G | 6.5 |
| A559N + Y579W + F638I | 1.6 |
| A559N + Y579W + K921R | 1.4 |
| A559N + Y579W + S616D + K921R | 7.4 |
| A559N + Y579W + S636N | 1.8 |
| A559N + Y579F | 1.7 |
| A559N + Y579W | 4.2 |
| A559N + Y579W + K921R | 5.8 |
| A559N + Y579W + S616D | 5.7 |
| F638I + Y934G | 1.8 |
| K627R + S636N | 1.5 |
| K627R + Y934G | 2.8 |
| P570K + Y579W | 3.1 |
| Q416D + A559N + Y579W + S636N | 3.9 |
| S128X + A559N + Y579W + K627R | 12.7 |
| S128X + A559N + Y579W + S636N | 7.6 |

### Example 4

### Half-Life Improvement Factors (HIF) of Endoglucanase Vvariants with Mutations in Chelator-Induced Instability Regions and Adjacent Regions

Variants of the mature parent endoglucanase of SEQ ID NO:2 were prepared and purified as described above in Examples 1 and 2. For the purposes of this example, variants were produced having mutations in at least one chelator-induced instability region (regions 1, 2, 3, 4, 5, 6, 7, 8, 9) and/or in at least one adjacent region (regions 10, 11, 12, 13, 14, 15, 16, 17, 18, 19). The in-detergent stability of the variants was determined as described in Example 3 by measuring the enzymatic activity present in purified samples of the variants after incubation with detergent and a protease. Incubation was performed using a 90% or 95% concentration of Persil Universal Gel detergent (PUG), with incubation at a temperature of 25, 26, 28, or 30° C. and a variant incubation time ranging from 11/2 h to up to 720 h.

Half-lives and HIFs were calculated as described above in Example 3. In cases where the difference in stability between wild-type and variants was too big to accurately assess half-life for both wild-type and variant using the same incubation time, the incubation time for wild-type and variant is different, e.g. 1 h for wild-type and up to 720 h for the most stable variants.

Tables 8-12 below show the HIFs for the purified variants along with information on the test conditions (incubation temperature, detergent concentration, incubation time) for each variant. HIFs of all variants were calculated based on the wild-type endoglucanase (SEQ ID NO:2) incubated at the same detergent concentration and temperature (HIF of wild-type=1 in all tables, per definition).

TABLE 8
Variants of the mature parent GH9 endoglucanase (SEQ ID NO: 2) with
corresponding half-life improvement factors (HIF) measured at a
temperature of 25° C.

| Alterations compared to | PUG (detergent | Incubation time | |
|---|---|---|---|
| reference (SEQ ID NO: 2) | concentration) | (hrs) | HIF |
| A885F | 90% PUG | 16 | 1.1 |
| S595L | 90% PUG | 16 | 1.1 |
| T292A | 90% PUG | 18 | 1.1 |
| F638I | 90% PUG | 16 | 1.5 |
| P570G + V837I | 90% PUG | 16 | 2.1 |

TABLE 9
Variants of the mature parent GH9 endoglucanase (SEQ ID NO: 2)
with corresponding half-life improvement factors (HIF) measured
at a temperature of 26° C.

| Alterations compared to | PUG (detergent | Incubation | |
|---|---|---|---|
| reference (SEQ ID NO: 2) | concentration) | time (hrs) | HIF |
| S593N | 90% PUG | 18 | 1.1 |
| A957L | 90% PUG | 18 | 1.1 |
| A942P | 90% PUG | 18 | 1.2 |
| K281R | 90% PUG | 18.15 | 1.2 |
| T932A | 90% PUG | 18 | 1.4 |
| V938I | 90% PUG | 18 | 1.4 |
| N933S | 90% PUG | 18 | 1.5 |
| A937E | 90% PUG | 18 | 1.6 |
| Y884H | 90% PUG | 18 | 1.9 |
| N941S | 90% PUG | 18 | 2.1 |
| F638I | 90% PUG | 18.5 | 2.2 |
| Y579W + K451Q | 90% PUG | 18 | 2.3 |
| K939V | 90% PUG | 18 | 2.5 |
| T711S + Y579W | 90% PUG | 19.5 | 2.6 |
| Y579W + K451S + T972K | 90% PUG | 18 | 2.8 |
| K713R + Y579W | 90% PUG | 19.5 | 2.9 |
| A564I | 90% PUG | 18 | 2.9 |

TABLE 10
Variants of the mature parent GH9 endoglucanase (SEQ ID NO: 2)
with corresponding half-life improvement factors (HIF) measured at a
temperature of 28° C.

| Alterations compared to | PUG (detergent | Incubation | |
|---|---|---|---|
| reference (SEQ ID NO: 2) | concentration) | time (hrs) | HIF |
| Y579W + Q834E | 90% PUG | 5.5 | 3.5 |
| Y579W + E408D | 90% PUG | 5.5 | 3.9 |

TABLE 11
Variants of the mature parent GH9 endoglucanase (SEQ ID NO: 2) with
corresponding half-life improvement factors (HIF) measured at a temperature of 30° C.

| Alterations compared to reference | PUG (detergent | Incubation | |
|---|---|---|---|
| (SEQ ID NO: 2) | concentration) | time (hrs) | HIF |
| A559N + Y579W + T639G | 90% PUG | 16 | 3.5 |
| A559N + Y579W + T639I + R898Q | 90% PUG | 16 | 3.5 |
| K451S + K627R | 90% PUG | 16 | 3.8 |
| Q4165 + F638I | 90% PUG | 16 | 3.9 |
| V4T + A559N + Y579W | 90% PUG | 16 | 3.9 |
| F638I + K921R | 90% PUG | 16 | 4.0 |
| Q4165 + S636N | 90% PUG | 16 | 4.1 |
| S636N + K921R | 90% PUG | 16 | 4.1 |
| Y579W + S636N | 90% PUG | 16 | 4.1 |
| Y55M + K627R + Y1042N | 90% PUG | 16 | 4.5 |
| A559N + Y579W + S636L | 90% PUG | 16 | 4.7 |
| H149X + A559N + K627R | 90% PUG | 16 | 5.2 |
| A448W + A559N + Y579W | 90% PUG | 16 | 5.7 |
| K627R + K921R | 90% PUG | 16 | 5.9 |
| E53Y + N216R + K627R | 90% PUG | 16 | 6.1 |
| E53Y + K627R | 90% PUG | 16 | 6.3 |
| K627R + F638I | 90% PUG | 16 | 6.5 |
| A448E + Y934G | 90% PUG | 16 | 6.7 |
| A559N + Y579W + A651P + S835A | 90% PUG | 168 | 6.7 |
| P570K + F638I | 90% PUG | 16 | 7.0 |
| E53Y + N216R + Y934G | 90% PUG | 16 | 7.4 |
| F20P + F638I | 90% PUG | 16 | 7.4 |
| K4515 + A559N + Y579W + S636N | 90% PUG | 168 | 8.5 |
| F20P + A559N + Y579W | 90% PUG | 16 | 8.7 |
| K4515 + A559N + Y579W + S636N | 90% PUG | 168 | 9.0 |
| E4085 + A559N + Y579W | 90% PUG | 16 | 9.6 |
| Y579W + K627R | 90% PUG | 16 | 9.9 |
| P570T + Y934G | 90% PUG | 16 | 11.4 |
| K512P + P570K | 90% PUG | 168 | 11.6 |
| P570Q + K627R | 90% PUG | 16 | 11.6 |
| A559N + Y579W + K627R | 90% PUG | 16 | 11.7 |
| A559N + Y579W + I602T + V603P | 90% PUG | 168 | 13.2 |
| P570T + E408D | 90% PUG | 16 | 14.3 |
| F20P + K627R | 90% PUG | 16 | 16.1 |
| A559N + Y579W + I602T + V603P + S616D | 90% PUG | 168 | 17.3 |
| A559N + Y579W + I602T + V603P + S616D + K921R | 90% PUG | 168 | 18.0 |
| E408D + A651P | 90% PUG | 16 | 18.4 |
| P570Q + Y579W | 90% PUG | 16 | 19.0 |
| Y55M + A559N + Y579W + A651P + Y934G | 90% PUG | 168 | 19.6 |
| K51Q + K627R + Y934G | 90% PUG | 16 | 19.8 |
| E408D + A559N + P570A + Y579W + K921R | 90% PUG | 16 | 20.7 |
| E408D + K512P + A559N + Y579W + F638I + A885F + K921R | 90% PUG | 16 | 20.7 |
| E408D + Q4165 + A559N + Y579W + S616D + K921R + Y934G | 90% PUG | 16 | 20.7 |
| F20N + K627R + S636N + A651P + K921R + Y934G | 90% PUG | 16 | 20.7 |
| F20N + S616D + K627R + A651P + K921R + Y934G | 90% PUG | 16 | 20.7 |
| F20N + S616D + K627R + S636N + A651P + Y934G | 90% PUG | 16 | 20.7 |
| K627R + S636N + A651P + K921R + Y934G | 90% PUG | 16 | 20.7 |
| P570K + K627R + F638I | 90% PUG | 16 | 20.7 |
| P570Q + E408D | 90% PUG | 16 | 20.7 |
| P570R + K627R + S636N | 90% PUG | 16 | 20.7 |
| P570S + K627R + S636N | 90% PUG | 16 | 20.7 |
| S616D + K627R + S636N + A651P + K921R + Y934G | 90% PUG | 16 | 20.7 |
| A559N + S560P + Y579W + K627R | 90% PUG | 16 | 21.3 |
| E408D + A559N + Y579W + S636N + K921R + Y934G | 90% PUG | 16 | 21.7 |
| E408D + A559N + Y579W + S616D + K921R | 90% PUG | 16 | 22.2 |
| K627R + A651P + Y934G | 90% PUG | 138 | 24.4 |
| F20P + F638I | 90% PUG | 138 | 25.7 |
| S616D + K627R + K921R + Y934G | 90% PUG | 138 | 25.7 |
| F20N + E408D + A559N + Y579W + K921R + Y934G | 90% PUG | 16 | 25.7 |
| E408D + Y934G | 90% PUG | 16 | 25.9 |
| E408D + Q416D + A559N + Y579W + I602T + V603P + | 90% PUG | 16 | 26.0 |
| S616D + K921R + Y934G | | | |
| F20P + E53Y + Q416D + A448E + K627R + Y934G | 90% PUG | 138 | 26.1 |
| E408D + A559N + Y579W + S636N + K921R | 90% PUG | 16 | 26.1 |
| V56M + K627R + Y934G | 90% PUG | 138 | 26.9 |
| E53Y + Y55M + E408D + A559N + Y579W + S636N + | 90% PUG | 16 | 27.8 |
| K921R + Y934G | | | |
| K627R + F638I + Y934G | 90% PUG | 138 | 27.8 |
| K51Q + K627R + Y934G | 90% PUG | 138 | 28.0 |
| K451S + A559N + Y579W + I602T + V603P + S636N | 90% PUG | 168 | 28.1 |
| S616D + K627R + A651P + K921R + Y934G + G994D | 90% PUG | 138 | 28.4 |
| F20N + S616D + K627R + S636N + Y934G | 90% PUG | 138 | 28.5 |
| A448E + K627R + Y934G | 90% PUG | 138 | 28.5 |
| E408D + K451S + A651P | 90% PUG | 16 | 28.6 |
| K627R + S636N + Y934G | 90% PUG | 138 | 28.7 |
| K627R + K921R + Y934G | 90% PUG | 138 | 28.9 |
| S6070 + S1038G | 90% PUG | 138 | 29.0 |
| K627R + S636K + Y934G | 90% PUG | 138 | 30.0 |
| F20P + Q416D + A559N + Y579W + K627R | 90% PUG | 138 | 30.7 |
| A559N + Y579W + K627R + Y934G | 90% PUG | 138 | 30.9 |
| F20N + S616D + K627R + Y934G | 90% PUG | 138 | 30.9 |
| F20N + E408D + A559N + Y579W + S616D + K921R + Y934G | 90% PUG | 16 | 31.3 |
| P570Q + Y579W | 90% PUG | 138 | 31.5 |
| K627R + Q834E + Y934G | 90% PUG | 138 | 31.6 |
| Q416S + A448W + A559N + K627R + Y934G | 90% PUG | 138 | 31.9 |
| P570K + K627R + Y934G | 90% PUG | 138 | 32.3 |
| A559N + Y579W + S616D + K627R | 90% PUG | 138 | 32.9 |
| F20N + S616D + K627R + A651P + K921R + Y934G | 90% PUG | 138 | 33.4 |
| F20N + E408D + A559N + Y579W + K627R + K921R + Y934G | 90% PUG | 16 | 33.8 |
| K512P + K627R + Y934G | 90% PUG | 138 | 33.8 |
| S616D + K627R + A651P + K921R + Y934G | 90% PUG | 138 | 34.6 |
| S616D + K627R + S636N + A651P + K921R + Y934G | 90% PUG | 138 | 34.9 |
| F20P + A448E + K627R + Y934G | 90% PUG | 138 | 35.5 |
| K627R + S636N + A651P + K921R + Y934G | 90% PUG | 138 | 36.1 |
| F20N + E408D + Q416S + A559N + Y579W + S616D + | 90% PUG | 16 | 36.1 |
| K921R + Y934G | | | |
| P570K + K627R + F638I | 90% PUG | 138 | 36.1 |
| S616D + K627R + S636N + K921R + Y934G | 90% PUG | 138 | 36.5 |
| F20N + K627R + Y934G | 90% PUG | 138 | 36.9 |
| P570T + K627R + Y934G | 90% PUG | 138 | 36.9 |
| F20P + E53Y + Q416D + K627R + Y934G | 90% PUG | 138 | 37.1 |
| F20P + E53Y + K627R + Y934G | 90% PUG | 138 | 37.3 |
| P570Q + K627R + Y934G | 90% PUG | 138 | 38.1 |
| P570K + Y579W + Y934Q | 90% PUG | 138 | 38.1 |
| F20N + S616D + K627R + S636N + A651P + Y934G | 90% PUG | 138 | 39.4 |
| P570S + K627R + S636N | 90% PUG | 138 | 39.6 |
| A559N + Y579W + S616D + K627R + S636N | 90% PUG | 138 | 40.1 |
| S616D + K627R + S636N + A651P + Y934G | 90% PUG | 138 | 41.5 |
| Q416D + P570K + Y579W | 90% PUG | 138 | 41.9 |
| A559N + Y579W + S616D + K627R + A651P | 90% PUG | 138 | 42.2 |
| A559N + Y579W + S616D + K627R + A651P | 90% PUG | 168 | 42.2 |
| P570K + Y579W + A651P | 90% PUG | 138 | 43.0 |
| P570Q + K627R + A651P | 90% PUG | 138 | 43.0 |
| P570R + K627R + S636N | 90% PUG | 138 | 43.5 |
| F20N + S616D + K627R + K921R + Y934G | 90% PUG | 138 | 43.7 |
| E408N + Q416D + K627R + S636N | 90% PUG | 138 | 44.4 |
| F20P + A559N + Y579W + K627R | 90% PUG | 138 | 44.4 |
| A559N + Y579W + S616D + K627R + S636N + A651P | 90% PUG | 168 | 44.5 |
| P570K + Y579W + Y934G | 90% PUG | 138 | 44.5 |
| P570Q + K627R + Y934R | 90% PUG | 138 | 44.5 |
| Q416S + P570K + Y579W | 90% PUG | 168 | 44.7 |
| A559N + Y579W + S616D + K627R + S636N + A651P | 90% PUG | 138 | 44.9 |
| F20N + A559N + Y579W + S616D + K627R + A651P | 90% PUG | 138 | 44.9 |
| F20P + E53Y + A559N + Y579W + K627R | 90% PUG | 138 | 46.2 |
| K627R + S636N + Q416D + E408N + Q416S | 90% PUG | 138 | 46.2 |
| P570K + Y579W + K927R | 90% PUG | 138 | 46.6 |
| E408D + S636N + A651P + N441G | 90% PUG | 168 | 46.7 |
| F20P + Q416D + K627R + Y934G | 90% PUG | 138 | 47.9 |
| P570K + Y579W + Q416D | 90% PUG | 138 | 48.8 |
| A559N + S560P + Y579W + K627R | 90% PUG | 138 | 49.9 |
| F20N + K627R + S636N + K921R + Y934G | 90% PUG | 138 | 50.1 |
| E408D + A559N + Y579W + I602T + V603P + S616D + K921R | 90% PUG | 16 | 50.1 |
| F20N + A559N + Y579W + S616D + K627R + S636N + A651P | 90% PUG | 138 | 52.1 |
| K512P + P570K + Y579W | 90% PUG | 138 | 52.3 |
| F20N + K627R + S636N + A651P + K921R + Y934G | 90% PUG | 138 | 52.4 |
| E408D + S636N + A651P + 1575V | 90% PUG | 168 | 52.7 |
| F20N + S616D + K627R + S636N + S642N + K921R + Y934G | 90% PUG | 138 | 52.9 |
| P570K + Y579W + S636N | 90% PUG | 138 | 53.6 |
| E408D + A559N + Y579W + K921R | 90% PUG | 16 | 54.1 |
| A559N + P570S + Y579W + K627R | 90% PUG | 138 | 55.3 |
| A559N + S560P + Y579W + K627R | 90% PUG | 138 | 55.5 |
| E408S + A559N + Y579W + K627R | 90% PUG | 138 | 55.7 |
| P570K + Y579W + K627M | 90% PUG | 138 | 55.9 |
| E408D + S636N + A651P + A924D | 90% PUG | 168 | 56.5 |
| E408D + Y579W | 90% PUG | 16 | 56.5 |
| E408N + Q416D + Y579W + K627R | 90% PUG | 138 | 57.3 |
| A559N + P570T + Y579W + K627R | 90% PUG | 138 | 58.0 |
| E408D + S636N + A651P + A688G | 90% PUG | 168 | 58.1 |
| P570Q + E408D | 90% PUG | 138 | 58.4 |
| E408D + S636N + A651P + V756H | 90% PUG | 168 | 58.5 |
| E408N + K627R + S636N | 90% PUG | 138 | 58.8 |
| E408D + Y579W + A937E + F20P + T887S + S636K | 90% PUG | 168 | 59.9 |
| E408D + S636N + A651P + T883R + Q956Y | 90% PUG | 168 | 60.4 |
| E408D + S636N + A651P + N216D | 90% PUG | 168 | 60.5 |
| E408D + A559N + Y579W + S616D + K921R + Y934R + | | | |
| A937E + K939V | 90% PUG | 138 | 60.7 |
| F20N + E408D + Q416S + A559N + Y579W + S616D + K921R | 90% PUG | 138 | 60.9 |
| E408D + Q416S + A559N + Y579W + S616D + K921R + Y934G | 90% PUG | 138 | 61.2 |
| A559N + P570R + Y579W + K627R | 90% PUG | 138 | 61.8 |
| E408D + Y934G | 90% PUG | 138 | 61.8 |
| F20P + E408D + Y579W + S636K + A937E | 90% PUG | 168 | 62.1 |
| E408D + S636N + A651P + K720H | 90% PUG | 168 | 62.1 |
| F20N + E408D + A559N + Y579W + K921R + Y934G + T883R | 90% PUG | 168 | 63.2 |
| E408D + A559N + Y579W + S616D + K921R + Q298E | 90% PUG | 138 | 63.5 |
| E408D + A559N + Y579W + S616D + K921R + Y934R + | | | |
| A937E + K939V | 90% PUG | 138 | 63.7 |
| E408D + Y579W + A651P + I602T + Q416S + S560P | 90% PUG | 168 | 63.8 |
| E408D + Y579W + A937E + F20P + S636K | 90% PUG | 168 | 63.8 |
| E408D + A559N + Y579W + I602T + V603P + S616D + | 90% PUG | 138 | 64.9 |
| K921R + Y934G + A885Q | | | |
| E408D + A559N + Y579W + K921R | 90% PUG | 168 | 65.1 |
| K627R + S636N + Q416D + E408N + Y579W | 90% PUG | 138 | 66.2 |
| E408D + K512P + A559N + Y579W + F638I + A885F + K921R | 90% PUG | 138 | 66.3 |
| E408D + Y579W + A651P + I602T + Q416S + V926P | 90% PUG | 168 | 66.5 |
| E408D + A559N + Y579W + S616D + K921R + Y934G + | 90% PUG | 138 | 66.6 |
| A937E + K939V | | | |
| E408D + A559N + P570A + Y579W + K921R | 90% PUG | 138 | 67.0 |
| E408D + A559N + Y579W + S616D + K921R + D635A | 90% PUG | 138 | 67.6 |
| E408D + A559N + Y579W + S616D + K921R | 90% PUG | 138 | 67.7 |
| S17A + F20P + Q416D + P570K + Y579W | 90% PUG | 138 | 68.0 |
| E408D + Y579W + A937E + F20P + S560P | 90% PUG | 138 | 69.1 |
| E408N + Q416D + P570K + Y579W | 90% PUG | 138 | 69.5 |
| F20N + E408D + Q416S + A559N + Y579W + S616D + | 90% PUG | 138 | 70.3 |
| K921R + Y934G | | | |
| A559N + Y579W + P570K + Y934R + Q416S | 90% PUG | 168 | 70.3 |
| P570K + Y579W + Q416D + E408N + S17A + F20P | 90% PUG | 138 | 70.5 |
| E408D + A559N + Y579W + I602T + V603P + S616D + | 90% PUG | 168 | 71.6 |
| K921R + Y934G + Y884H | | | |
| E408D + A559N + Y579W + S616D + K921R + Y934G | 90% PUG | 138 | 71.9 |
| A559N + P570Q + Y579W + K627R | 90% PUG | 138 | 72.1 |
| E408D + Q416D + A559N + Y579W + K921R + Y934G | 90% PUG | 138 | 72.1 |
| F20N + P570K + Y579W | 90% PUG | 138 | 72.9 |
| A559N + Y579W + P570K + Y934R + Q416D | 90% PUG | 168 | 72.9 |
| A559N + Y579W + P570K + Y934R + F20P | 90% PUG | 168 | 74.9 |
| E408D + Y579W + A651P + I602T + Q416S + V756Y | 90% PUG | 168 | 75.4 |
| E408D + A559N + Y579W + I602T + V603P + S616D + | 90% PUG | 138 | 75.9 |
| K921R + Y934G + A885F | | | |
| E408D + Y579W + A651P + I602T + Q416S + I403Y | 90% PUG | 168 | 76.1 |
| E408D + A559N + Y579W + I602T + V603P + S616D + | 90% PUG | 138 | 76.2 |
| K921R + Y934G + Q834E | | | |
| E408D + A559N + Y579W + S616D + K921R + S313D | 90% PUG | 138 | 76.2 |
| E408D + K451S + A651P | 90% PUG | 138 | 76.2 |
| E408D + S636N + A651P + H182Y | 90% PUG | 138 | 76.6 |
| E408D + Y579W + A937E + F20P + V603P | 90% PUG | 138 | 77.8 |
| E408D + A559N + Y579W + K921R | 90% PUG | 138 | 77.9 |
| Y579W + A937E | 90% PUG | 138 | 77.9 |
| E408D + Q416D + A559N + Y579W + K627R + K921R + GY934 | 90% PUG | 138 | 78.0 |
| E408D + S636N + A651P + K631R | 90% PUG | 138 | 78.2 |
| A559N + Y579W + P570K + Y934R + K627R | 90% PUG | 168 | 78.7 |
| P570K + Y579W + Q416D + S17A + F20P + E408N + Q416D | 90% PUG | 138 | 79.1 |
| E53Y + Y55M + E408D + A559N + Y579W + S636N + | 90% PUG | 138 | 80.1 |
| K921R + Y934G | | | |
| E408D + A559N + Y579W + K921R + Y934G + T697G + F20N | 90% PUG | 138 | 80.1 |
| E408D + Y579W + A651P + I602T + Q416S + V881Q | 90% PUG | 168 | 80.1 |
| E408D + S636N + A651P + T999R | 90% PUG | 138 | 81.0 |
| E408D + A651P | 90% PUG | 138 | 81.1 |
| E408D + A559N + Y579W + S636N + K921R | 90% PUG | 138 | 81.6 |
| E408D + Q416D + A559N + P570R + Y579W + K921R + Y934G | 90% PUG | 138 | 82.3 |
| E408D + Y579W + Q834E | 90% PUG | 138 | 82.5 |
| F20N + E408D + A559N + Y579W + K921R + Y934G | 90% PUG | 138 | 82.7 |
| A559N + Y579W + P570K + Y934R + A651P | 90% PUG | 138 | 83.2 |
| E408D + S636N + A651P + F1048W | 90% PUG | 138 | 83.3 |
| E408D + Y579W + A651P + I602T + Q416S + Y579W | 90% PUG | 168 | 83.9 |
| E408D + A559N + P570Q + Y579W + K921R + Y934G | 90% PUG | 138 | 84.1 |
| E408D + S636N + A651P + R880K | 90% PUG | 138 | 84.1 |
| F20P + E408D + Y579W + A937E | 90% PUG | 138 | 84.2 |
| E408D + A559N + Y579W + I602T + V603P + S616D + | 90% PUG | 138 | 84.5 |
| K921R + A1037E | | | |
| E408D + A559N + Y579W + S616D + K921R + T697G + A885Q | 90% PUG | 138 | 84.5 |
| E408D + Y579W + A937E + F20P + T883R | 90% PUG | 138 | 84.7 |
| E408D + Y579W + A651P + I602T + Q416S + K627R | 90% PUG | 168 | 85.1 |
| F20N + E408D + A559N + Y579W + K921R + Y934G + T999R | 90% PUG | 138 | 85.3 |
| E408D + Y579W + A937E + F20P + Q834E | 90% PUG | 138 | 85.5 |
| P570K + Y579W + Q416D + S17A + F20P + Y934R | 90% PUG | 138 | 87.1 |
| E408D + S636N + A651P | 90% PUG | 138 | 87.6 |
| P570K + Y579W + Q416D + S17A + F20P + I602T | 90% PUG | 138 | 87.6 |
| E408D + S636N + A651P + Q298E | 90% PUG | 138 | 88.0 |
| E408D + Y579W + A651P + I602T + Q416S + A559N | 90% PUG | 168 | 88.1 |
| E408D + S636N + A651P + V926P | 90% PUG | 138 | 88.9 |
| E408D + A559N + Y579W + I602T + V603P + S616D + | 90% PUG | 138 | 88.9 |
| K921R + Y934G + S636K | | | |
| E408D + A559N + Y579W + S636N + A651P + K921R + Y934G | 90% PUG | 138 | 89.0 |
| E408D + A559N + Y579W + S616D + K921R + V756Y | 90% PUG | 138 | 89.5 |
| P570K + Y579W + Q416D + S17A + F20P + I602T + V603P | 90% PUG | 138 | 89.5 |
| E408D + A559N + Y579W + S636N + K921R + Y934G | 90% PUG | 138 | 90.6 |
| E408D + Y579W + A937E + F20P + S636K | 90% PUG | 138 | 90.8 |
| E408D + Q416D + A559N + Y579W + I602T + V603P + | 90% PUG | 1 38 | 90.9 |
| S616D + K921R + Y934G + A651P + A885F | | | |
| E408D + A559N + Y579W + I602T + V603P + S616D + | 90% PUG | 138 | 91.1 |
| K921R + Y934G + S17A | | | |
| E408D + A559N + Y579W + I602T + V603P + S616D + | 90% PUG | 138 | 91.5 |
| K921R + Y934G | | | |
| E408D + A559N + Y579W + S616D + T697G + V756Y + K921R | 90% PUG | 138 | 91.5 |
| E408D + Y579W + A651P + I602T + Q416S + S17A + F20P | 90% PUG | 168 | 93.5 |
| F20N + E408D + A559N + Y579W + K627R + K921R + Y934G | 90% PUG | 138 | 93.9 |
| A559N + Y579W + P570K + Y934R + E408D | 90% PUG | 168 | 94.3 |
| E408D + Q416D + A559N + Y579W + K921R | 90% PUG | 168 | 94.7 |
| E408D + A559N + Y579W + K921R + Y934G + A937E + K939V | 90% PUG | 138 | 95.0 |
| E408D + S636N + A651P + V756H + T883R | 90% PUG | 168 | 95.0 |
| E408D + Y579W + A937E + F20P + S17A + F20P | 90% PUG | 138 | 95.0 |
| E408D + A559N + Y579W + I602T + V603P + S616D + K921R | 90% PUG | 138 | 95.5 |
| F20N + E408D + A559N + Y579W + K921R + Y934G + I302D | 90% PUG | 138 | 95.9 |
| E408D + Q416S + Y579W + I602T + A651P + R880K | 90% PUG | 138 | 96.7 |
| F20P + E408D + Q416S + A559N + S560P + P570R + | 90% PUG | 168 | 97.7 |
| Y579W + K627R + K921R + Y934R | | | |
| F20P + E408D + A559N + Y579W + K921R + Y934G | 90% PUG | 138 | 98.0 |
| E408D + A559N + Y579W + I602T + V603P + S616D + | 90% PUG | 168 | 98.1 |
| K921R + Y934G + A937E + K939V | | | |
| E408D + S636N + A651P + T883K | 90% PUG | 138 | 98.5 |
| N18G + A71E + L226K + E408D + Y579W + I602T + A651P + | 90% PUG | 168 | 98.5 |
| A688G + V756Y + T887S | | | |
| E408D + A559N + Y579W + I602T + V603P + S616D + | 90% PUG | 138 | 101.3 |
| K921R + Y934R + A937E + K939V | | | |
| E408D + A559N + Y579W + I602T + V603P + S616D + K921R | 90% PUG | 138 | 102.0 |
| E408D + A559N + Y579W + S616D + K921R + T697G + A65I | 90% PUG | 138 | 102.2 |
| F20N + E408D + A559N + Y579W + K921R + Y934G + R880K | 90% PUG | 138 | 102.2 |
| A559N + Y579W + P570K + Y934R + E408N | 90% PUG | 138 | 102.9 |
| E408D + Y579W + A651P | 90% PUG | 138 | 103.7 |
| P570K + Y579W + Q416D + S17A + F20P + A651P | 90% PUG | 138 | 103.7 |
| F20P + E408D + Q416D + A559N + S560P + P570R + | 90% PUG | 168 | 103.8 |
| Y579W + K627R + S636N + F638I + K921R + Y934R | | | |
| E408D + A559N + Y579W + S616D + T697G + K921R | 90% PUG | 138 | 104.1 |
| F20P + E408D + Y579W + S636K + T697G + A937E | 90% PUG | 168 | 105.4 |
| E408D + A559N + Y579W + I602T + V603P + S616D + | 90% PUG | 138 | 106.4 |
| K921R + N933S + Y934R + A937E + V9381 + K939V + | | | |
| N941S + A942P | | | |
| F20P + E408D + Y579W + A651P | 90% PUG | 138 | 106.9 |
| K51Q + E408D + Y579W + A651P | 90% PUG | 138 | 109.7 |
| A559N + Y579W + P570K + Y934R + Q683E | 90% PUG | 138 | 109.9 |
| F20P + E408D + Q416D + A559N + S560P + P570Q + | 90% PUG | 168 | 109.9 |
| Y579W + K627R + K921R + Y934G | | | |
| E408D + Q416D + A559N + Y579W + I602T + V603P + | 90% PUG | 168 | 110.9 |
| S616D + A651P + K921R + Y934G | | | |
| E408D + A559N + Y579W + S616D + K921R + T697G + V881T | 90% PUG | 138 | 111.8 |
| E408D + A559N + Y579W + S636N + Q683E + K921R + Y934G | 90% PUG | 138 | 112.7 |
| E408D + S636N + A651P + V756Y | 90% PUG | 138 | 113.4 |
| F20P + E408D + Y579W + S636K + A937E | 90% PUG | 168 | 113.9 |
| E408D + S636N + A651P + A885Q | 90% PUG | 138 | 114.4 |
| E408D + S636N + A651P + V756H + T883K | 90% PUG | 168 | 118.9 |
| F20P + E408D + Q416S + A559N + S560P + P570R + | 90% PUG | 168 | 121.5 |
| Y579W + K627R + S636N + F638I + K921R + Y934G | | | |
| E408D + Y579W + I602T + A651P | 90% PUG | 138 | 122.1 |
| E408D + S636N + A651P + T697G + T883R | 90% PUG | 168 | 123.5 |
| E408D + S636N + A651P + V756H + R880K | 90% PUG | 168 | 124.3 |
| E408D + Y579W + A651P + I602T + R880K | 90% PUG | 168 | 124.5 |
| F20N + E408D + A559N + Y579W + K921R + Y934G + | 90% PUG | 138 | 125.1 |
| T697G + Y55M | | | |
| E408D + Q416D + A559N + Y579W + I602T + V603P + | 90% PUG | 138 | 125.4 |
| S616D + K921R + Y934G | | | |
| S17A + F20P + E408D + Y579W + I602T + A651P + | 90% PUG | 168 | 127.3 |
| F906A + Y934G | | | |
| F20N + E408D + A559N + Y579W + K921R + Y934G + T8875 | 90% PUG | 138 | 127.7 |
| E408D + S636N + A651P + V756H + Y579W | 90% PUG | 168 | 129.7 |
| E408D + S636N + A651P + S313D | 90% PUG | 138 | 132.5 |
| E408D + Y579W + A651P + I602T + V881T | 90% PUG | 138 | 135.9 |
| E408D + Y579W + A651P + I602T + T887S | 90% PUG | 138 | 138.8 |
| E408D + Y579W + I602T + F638N + A651P + R880K + | 90% PUG | 168 | 139.5 |
| K921R + Y934G | | | |
| E408D + S636N + A651P + T697G | 90% PUG | 138 | 140.2 |
| E408D + S636N + A651P + V756H + I602T + V603P | 90% PUG | 168 | 140.7 |
| E408D + A559N + Y579W + S616D + K921R + T697G + | 90% PUG | 138 | 141.8 |
| I602T + V603P | | | |
| E408D + Q416D + A559N + Y579W + I602T + V603P + | 90% PUG | 138 | 142.7 |
| S616D + K921R + Y934G + A651P + Y884H | | | |
| F20N + E408D + A559N + Y579W + K921R + Y934G + V756Y | 90% PUG | 138 | 143.9 |
| E408D + S636N + A651P + V756H + A885Q | 90% PUG | 168 | 144.7 |
| S17A + F20P + E408D + Y579W + I602T + A651P + F906A + | 90% PUG | 168 | 150.2 |
| Y934G + S636K | | | |
| F20N + E408D + A559N + Y579W + K921R + Y934G + | 90% PUG | 138 | 150.6 |
| T697G + T887K | | | |
| F20N + E408D + A559N + Y579W + K921R + Y934G + | 90% PUG | 138 | 151.1 |
| T697G + A885Q | | | |
| E408D + Y579W + A651P + I602T + R880K | 90% PUG | 138 | 151.2 |
| S17A + F20P + E408D + Y579W + I602T + A651P + | 90% PUG | 168 | 151.5 |
| F906A + Y934G + V881Q | | | |
| I302D + E408D + Q4165 + Y579W + I602T + A651P + | 90% PUG | 168 | 152.0 |
| R880K + Y934Q | | | |
| S17A + F20P + E408D + Y579W + I602T + A651P + | 90% PUG | 168 | 153.5 |
| F906A + Y934G + L888M | | | |
| S17A + F20P + E408D + Y579W + I602T + A651P + | 90% PUG | 168 | 154.1 |
| F906A + Y934G + S560P | | | |
| I302D + E408D + Q4165 + Y579W + I602T + A651P + | 90% PUG | 168 | 154.9 |
| R880K + K744Q + N848D + A868E | | | |
| F20P + S313D + E408D + Y579W + S636K + T697G + | 90% PUG | 168 | 155.5 |
| T887K + A937E | | | |
| F20P + S313D + E408D + Y579W + S636K + T697G + | 90% PUG | 168 | 156.2 |
| A937E + A885Q | | | |
| E408D + A559N + Y579W + S616D + K921R + T697G + Y934G | 90% PUG | 138 | 156.9 |
| S17A + F20P + E408D + Y579W + I602T + A651P + | 90% PUG | 168 | 157.2 |
| F906A + Y934G + T887K | | | |
| E408D + Y579W + A651P + I602T + V881Q | 90% PUG | 138 | 157.6 |
| E408D + S636N + A651P + A688G + A283D | 90% PUG | 168 | 162.3 |
| S17A + F20P + E408D + Y579W + I602T + A651P + | 90% PUG | 168 | 163.7 |
| N905D + T825G | | | |
| A559N + Y579W + A688G + V756Y + K921R + Y934G | 90% PUG | 168 | 164.8 |
| E408D + S636N + A651P + V756H + Y934Q | 90% PUG | 168 | 165.3 |
| S17A + F20P + E408D + Y579W + I602T + A651P + F906A | 90% PUG | 168 | 166.2 |
| E408D + Q4165 + Y579W + I602T + A651P + N848D + A868E | 90% PUG | 168 | 168.9 |
| E408D + S636N + A651P + A688G + D476R | 90% PUG | 168 | 169.4 |
| I302D + E408D + Q4165 + Y579W + I602T + A651P + | 90% PUG | 168 | 171.5 |
| R880K + V756H | | | |
| S17A + F20P + E408D + Y579W + I602T + A651P + F906A + | 90% PUG | 168 | 172.3 |
| Y934G + S636N | | | |
| E408D + Q416D + A559N + Y579W + I602T + V603P + | 90% PUG | 138 | 176.9 |
| S616D + K921R + Y934G + A651P + T8875 | | | |
| S17A + F20P + E408D + Y579W + I602T + A651P + N848D | 90% PUG | 168 | 178.3 |
| E408D + Q416S + Y579W + I602T + A651P + V756Y | 90% PUG | 168 | 181.5 |
| E408D + Q4165 + Y579W + I602T + A651P + T697G | 90% PUG | 168 | 204.0 |

TABLE 12
Variants of the mature parent GH9 endoglucanase (SEQ ID NO: 2) with
corresponding half-life improvement factors (HIF) measured at a temperature of 30° C.

| | PUG (detergent | Incubation | |
|---|---|---|---|
| Alterations compared to reference (SEQ ID NO: 2) | concentration) | time (hrs) | HIF |
| E408D + A559N + Y579W + K921R + Y934R + A937E + K939V | 95% PUG | 168 | 72.0 |
| E408D + A559N + Y579W + K921R + Y934R + A937E + | 95% PUG | 168 | 74.7 |
| V9381 + K939V + N9415 + A942P | | | |
| E408D + Y579W + A651P + I602T + Q416S | 95% PUG | 168 | 80.0 |
| E408D + Y579W + A937E + F20P + Q683E | 95% PUG | 168 | 82.7 |
| E408D + S636N + A651P + A283D | 95% PUG | 336 | 91.3 |
| E408D + S636N + A651P + N18G | 95% PUG | 336 | 91.3 |
| E408D + Y579W + A651P + I602T + S17A + F20P | 95% PUG | 168 | 91.3 |
| E408D + S636N + A651P + A346D | 95% PUG | 336 | 96.7 |
| E408D + S636N + A651P + A71E + A120P + A186P + D247N | 95% PUG | 168 | 102.4 |
| F20N + E408D + A559N + Y579W + Q683E + K921R + Y934G | 95% PUG | 168 | 112.7 |
| E408D + Y579W + A651P + I602T + Y934Q | 95% PUG | 168 | 113.3 |
| E408D + S636N + A651P + T872G | 95% PUG | 336 | 116.7 |
| E408D + Y579W + A651P + I602T + T883R | 95% PUG | 168 | 120.0 |
| F20N + E408D + A559N + Y579W + T697G + K921R + Y934G | 95% PUG | 168 | 122.0 |
| E408D + Y579W + A651P + I602T + Q4165 + R880K + N216D | 95% PUG | 336 | 125.3 |
| E408D + A559N + Y579W + I602T + V603P + S616D + | 95% PUG | 168 | 129.5 |
| K921R + Y934G + Q683E | | | |
| E408D + Y579W + A651P + I602T + Q4165 + R880K + F20P | 95% PUG | 336 | 139.2 |
| E408D + S636N + A651P + N848D | 95% PUG | 336 | 141.7 |
| E408D + Y579W + A651P + I602T + Q4165 + R880K + I302D | 95% PUG | 336 | 148.8 |
| E408D + Y579W + I602T + F638N + A651P + R880K + K921R + | 95% PUG | 672 | 152.9 |
| Y934G + K51Q | | | |
| F20N + E408D + A559N + Y579W + K921R + Y934G + | 95% PUG | 168 | 156.0 |
| T697G + K512P | | | |
| E408D + Y579W + A651P + I602T + Q4165 + R880K + Y884H | 95% PUG | 336 | 156.9 |
| N18G + A120P + E408D + S636N + A651P + A688G + V756Y | 95% PUG | 672 | 161.5 |
| E408D + S636N + A651P + V756H + S313D | 95% PUG | 750 | 161.7 |
| N18G + A71E + A186P + E408D + Y579W + I602T + A651P + | 95% PUG | 672 | 161.7 |
| A688G + V756Y | | | |
| E408D + S636N + A651P + V756H + T697G | 95% PUG | 672 | 164.9 |
| E408D + Y579W + A651P + I602T + T883R + V756Y | 95% PUG | 750 | 167.6 |
| F20P + E408D + Y579W + 5636K + T697G + A937E + V926F | 95% PUG | 336 | 168.4 |
| E408D + A559N + Y579W + S616D + K921R + T697G + I602T | 95% PUG | 672 | 168.9 |
| N216D + I302D + E408D + Q4165 + Y579W + I602T + A651P + | 95% PUG | 672 | 170.3 |
| A688G + R880K + V890R + N905D | | | |
| E408D + Y579W + I602T + F638N + A651P + N848D + | 95% PUG | 168 | 172.6 |
| R880K + K921R + Y934G | | | |
| N18G + N189K + E408D + A559N + Y579W + A688G + | 95% PUG | 750 | 175.7 |
| T697G + V756Y + K921R + Y934G | | | |
| E408D + Y579W + I602T + F638N + A651P + V756Y + R880K + | 95% PUG | 672 | 181.0 |
| K921R + Y934G + P570R | | | |
| S313D + E408D + Y579W + I602T + F638N + A651P + R880K + | 95% PUG | 750 | 182.5 |
| K921R + Y934G | | | |
| E408D + Y579W + A651P + I602T + T883R + N848D | 95% PUG | 750 | 183.1 |
| E408D + Y579W + I602T + F638N + A651P + R880K + | 95% PUG | 672 | 183.3 |
| N905D + K921R + Y934G + A283D | | | |
| E408D + Y579W + I602T + F638N + A651P + R880K + | 95% PUG | 672 | 183.3 |
| N905D + K921R + Y934G + A885Q | | | |
| E408D + Y579W + I602T + F638N + A651P + R880K + | 95% PUG | 750 | 183.4 |
| N905D + K921R + Y934G | | | |
| S17A + F20P + E408D + Y579W + I602T + A651P + F906A + | 95% PUG | 750 | 183.7 |
| Y934G + S313D | | | |
| E408D + Y579W + I602T + F638N + A651P + R880K + K921R + | 95% PUG | 672 | 183.8 |
| Y934G + Y55D | | | |
| E408D + Y579W + I602T + F638N + A651P + R880K + K921R + | 95% PUG | 672 | 184.0 |
| Y934G + N848D | | | |
| S313D + I302D + E408D + Y579W + I602T + F638N + A651P + | 95% PUG | 672 | 186.3 |
| R880K + K921R + Y934G | | | |
| N18G + A186P + E408D + Q416S + Y579W + I602T + A651P + | 95% PUG | 672 | 186.8 |
| A688G + V756Y | | | |
| E408D + Y579W + A651P + I602T + T883R + A688G | 95% PUG | 750 | 188.3 |
| E408D + Y579W + I602T + A651P + T697G + T883R | 95% PUG | 672 | 189.9 |
| N18G + E408D + S636N + A651P + A688G + T697G + V756Y | 95% PUG | 750 | 190.9 |
| N18G + E408D + A559N + Y579W + I602T + V603P + S616D + | 95% PUG | 672 | 196.2 |
| T697G + V756Y + K921R + T999R | | | |
| F20N + A346D + E408D + K512P + A559N + Y579W + I602T + | 95% PUG | 672 | 197.8 |
| S636N + A651P + T697G + V756Y + A824D + A885N + | | | |
| K921R + Y934G | | | |
| F20N + E408D + Q489P + K512P + A559N + Y579W + I602T + | 95% PUG | 672 | 198.0 |
| A651P + T697G + V756Y + A885N + K921R + Y934G + A937E | | | |
| E408D + Y579W + I602T + F638N + A651P + R880K + K921R + | 95% PUG | 672 | 200.7 |
| Y934G + V756Y | | | |
| E408D + Y579W + I602T + F638N + A651P + R880K + | 95% PUG | 672 | 200.8 |
| N905D + K921R + Y934G + N848D | | | |
| S17A + F20P + E408D + Y579W + I602T + A651P + A688G | 95% PUG | 672 | 202.0 |
| E408D + S636N + A651P + A688G + N848D + V881Q + N905D | 95% PUG | 168 | 202.3 |
| F20N + E408D + K512P + A559N + Y579W + T697G + K921R + | 95% PUG | 336 | 202.3 |
| Y934G + A651P | | | |
| F20P + Y55M + S313D + E408D + Y579W + S636K + T697G + | 95% PUG | 672 | 202.8 |
| A937E | | | |
| I302D + E408D + Q4165 + Y579W + I602T + A651P + R880K + | 95% PUG | 672 | 204.5 |
| T697G | | | |
| N18G + A186P + E408D + Y579W + I602T + A651P + A688G + | 95% PUG | 750 | 204.5 |
| V756Y + V881T | | | |
| S17A + F20P + E408D + Y579W + I602T + A651P + F906A + | 95% PUG | 672 | 205.5 |
| Y934G + A688G | | | |
| I302D + S313D + E408D + Q4165 + Y579W + I602T + A651P + | 95% PUG | 672 | 206.3 |
| A688G + R880K + T892V + N905D + Q912V | | | |
| E408D + S636N + A651P + T697G + S313D | 95% PUG | 672 | 206.6 |
| F20P + S313D + E408D + Y579W + S636K + T697G + A937E + | 95% PUG | 672 | 207.3 |
| V603P | | | |
| F20P + S313D + E408D + Y579W + S636K + T697G + A937E + | 95% PUG | 672 | 207.5 |
| T887S | | | |
| E408D + Y579W + I602T + F638N + A651P + R880K + N905D + | 95% PUG | 672 | 207.9 |
| K921R + Y934G + A885N | | | |
| I302D + E408D + Q4165 + Y579W + I602T + A651P + A688G + | 95% PUG | 750 | 212.4 |
| R880K | | | |
| E408D + Y579W + I602T + F638N + A651P + V756Y + R880K + | 95% PUG | 168 | 214.2 |
| K921R + Y934G | | | |
| E408D + Y579W + I602T + F638N + A651P + A688G + | 95% PUG | 672 | 214.9 |
| K720H + R880K + K921R + Y934G | | | |
| I302D + E408D + Q4165 + Y579W + I602T + A651P + A688G + | 95% PUG | 672 | 215.5 |
| W719R + R880K + N905D + F1048W | | | |
| K51Q + E408D + Y579W + I602T + F638N + A651P + R880K + | 95% PUG | 672 | 216.0 |
| K921R + Y934G | | | |
| I302D + E408D + Q4165 + Y579W + I602T + A651P + A688G + | 95% PUG | 672 | 222.1 |
| W719R + R880K + N905D + F1048W | | | |
| I302D + E408D + Q416S + Y579W + I602T + A651P + A688G + | 95% PUG | 672 | 224.2 |
| V881Q + K921R | | | |
| Y55M + A283D + S313D + E408D + Y579W + I602T + F638N + | 95% PUG | 672 | 224.3 |
| A651P + K720H + V756Y + N848D + A869V + R880K + | | | |
| Q912V + K921R + Y934R | | | |
| F20P + S313D + E408D + V567P + Y579W + S636K + A651P + | 95% PUG | 672 | 224.6 |
| T697G + N848D + T883R + A937E + Q956Y + F1048W | | | |
| F20P + S313D + E408D + Y579W + S636K + T697G + A937E + | 95% PUG | 672 | 226.1 |
| T883C | | | |
| S313D + A346D + E408D + Y579W + I602T + F638N + A651P + | 95% PUG | 672 | 230.5 |
| T697G + N848D + R880K + A885N + K921R + Y934G | | | |
| E408D + Y579W + I602T + F638N + A651P + R880K + | 95% PUG | 672 | 231.7 |
| N905D + K921R + Y934G + A688G | | | |
| E408D + Y579W + I602T + F638N + A651P + V756Y + R880K + | 95%PUG | 672 | 232.3 |
| K921R + Y934G + N848D | | | |
| I302D + E408D + Q416S + Y579W + I602T + A651P + A688G + | 95% PUG | 672 | 232.9 |
| V881Q + K921R | | | |
| F20P + S313D + E408D + Y579W + S636K + T697G + A937E + | 95% PUG | 672 | 235.3 |
| A885N | | | |
| F20P + S313D + E408D + Y579W + S636K + T697G + A937E + | 95% PUG | 750 | 236.9 |
| E53Y | | | |
| F20P + S313D + E408D + Y579W + S636K + T697G + A937E + | 95% PUG | 750 | 238.2 |
| R880K | | | |
| S17A + F20P + E408D + Y579W + I602T + A651P + F906A + | 95% PUG | 750 | 240.0 |
| Y934G + T697G | | | |
| Y55M + A283D + S313D + E408D + Y579W + I602T + F638N + | 95% PUG | 672 | 243.1 |
| A651P + K720H + V756Y + N848D + A869V + R880K + | | | |
| N905D + K921R + Y934R | | | |
| S313D + E408D + S560P + Y579W + I602T + F638N + A651P + | 95% PUG | 672 | 244.9 |
| W719R + V756Y + T825G + N848D + A869V + D870V + | | | |
| R880K + N905D + K921R + Y934G | | | |
| F20P + S313D + E408D + Y579W + S636K + T697G + A937E + | 95% PUG | 750 | 247.1 |
| T883V | | | |
| S313D + E408D + S560P + Y579W + I602T + F638N + A651P + | 95% PUG | 672 | 250.9 |
| W719R + V756Y + N848D + A869V + D870V + R880K + | | | |
| K921R + Y934G | | | |
| I302D + E408D + Q4165 + Y579W + I602T + A651P + A688G + | 95% PUG | 672 | 253.3 |
| R880K + N905D | | | |
| F20P + S313D + E408D + Y579W + S636K + T697G + A937E | 95% PUG | 336 | 253.9 |
| E408D + Y579W + I602T + F638N + A651P + V756Y + R880K + | 95%PUG | 672 | 254.7 |
| K921R + Y934G + S313D | | | |
| E408D + Y579W + I602T + A651P + A688G + N848D + T883R | 95% PUG | 672 | 255.1 |
| S17A + F20P + N216D + I302D + E408D + Q4165 + Y579W + | 95% PUG | 672 | 257.1 |
| I602T + A651P + W719R + T883R + F906A + Y934G + Q956Y | | | |
| I302D + E408D + Q4165 + Y579W + I602T + A651P + A688G + | 95% PUG | 672 | 257.7 |
| R880K + N905D + K921R | | | |
| S313D + E408D + Y579W + I602T + F638N + A651P + T697G + | 95% PUG | 672 | 258.5 |
| R880K + K921R + Y934G | | | |
| I302D + E408D + Q4165 + Y579W + I602T + A651P + A688G + | 95% PUG | 672 | 259.4 |
| V881Q + N905D + K921R | | | |
| I302D + E408D + Q4165 + Y579W + I602T + A651P + A688G + | 95% PUG | 672 | 263.5 |
| V881Q + N905D + K921R | | | |
| E408D + Y579W + I602T + A651P + A688G + N848D + | 95% PUG | 672 | 268.3 |
| T883R + M980I | | | |
| S313D + E408D + Y579W + I602T + F638N + A651P + T697G + | 95% PUG | 672 | 272.3 |
| R880 K + K921R + Y934G | | | |
| S313D + E408D + S560P + Y579W + I602T + F638N + A651P + | 95% PUG | 672 | 272.7 |
| W719R + V756Y + N848D + A869V + D870V + R880 K + | | | |
| N905D + K921R + Y934G | | | |
| N216Q + I302D + H311N + E408D + Q416S + Y579W + I602T + | 95% PUG | 672 | 275.1 |
| A651P + A688G + T697G + W719R + R880K + N905D + Q912V | | | |
| F20P + S313D + E408D + Y579W + S636K + T697G + N848D + | 95% PUG | 750 | 285.8 |
| T887K + A937E | | | |
| S313D + E408D + Y579W + I602T + F638N + A651P + T697G + | 95% PUG | 672 | 288.9 |
| R880K + K921R + Y934G | | | |
| Y55M + A283D + S313D + E408D + Y579W + I602T + F638N + | 95% PUG | 672 | 289.4 |
| A651P + W719R + V756Y + N848D + A869V + R880K + | | | |
| K921R + Y934R | | | |
| F20N + E408D + K512P + A559N + Y579W + I602T + A651P + | 95% PUG | 672 | 290.1 |
| T697G + V756Y + K921R + Y934G | | | |
| Y55M + A283D + S313D + E408D + Y579W + I602T + F638N + | 95% PUG | 672 | 291.9 |
| A651P + W719R + V756Y + N848D + A869V + R880K + | | | |
| K921R + Y934R | | | |
| A283D + S313D + E408D + A559P + Y579W + I602T + F638N + | 95% PUG | 672 | 292.7 |
| A651P + W719R + V756Y + N848D + A869V + D870V + | | | |
| R880K + N905D + K921R + Y934G | | | |
| F20P + I302D + S313D + E408D + D476R + Y579W + S636K + | 95% PUG | 672 | 293.3 |
| T697G + V756Y + V881Q + T887K + F906A + A937E | | | |
| S313D + E408D + S560P + Y579W + I602T + F638N + A651P + | 95% PUG | 672 | 295.1 |
| W719R + V756Y + N848D + A869V + D870V + R880K + | | | |
| N905D + K921R + Y934G | | | |
| F20P + I302D + S313D + E408D + D476R + Y579W + S636K + | 95% PUG | 672 | 299.4 |
| T697G + V756Y + T887K + F906A + A937E | | | |
| S17A + F20P + N216D + A283D + H311N + E408D + Y579W + | 95% PUG | 672 | 300.1 |
| I602T + A651P + A688G + T883R + F906A + Y934G + Q956Y | | | |
| Y55M + A283D + S313D + E408D + A559P + Y579W + I602T + | 95% PUG | 672 | 303.3 |
| F638N + A651P + T697G + W719R + N848D + R880K + | | | |
| K921R + Y934G + A937E | | | |
| F20P + S313D + E408D + Y579W + S636K + T697G + N848D + | 95% PUG | 750 | 304.5 |
| T887K + A937E + G1041R | | | |
| F20P + S313D + E408D + V567P + Y579W + S636K + T697G + | 95% PUG | 672 | 307.5 |
| N848D + A937E + Q956Y | | | |
| F20P + I302D + S313D + E408D + D476R + Y579W + S636K + | 95% PUG | 672 | 309.7 |
| T697G + V756Y + V881Q + T887K + F906A + A937E | | | |
| I302D + E408D + Q416S + Y579W + I602T + A651P + A688G + | 95% PUG | 672 | 317.0 |
| W719R + R880K + N905D + F1048W | | | |
| S17A + F20P + N216D + A283D + H311N + E408D + S560P + | 95% PUG | 672 | 319.1 |
| Y579W + I602T + A651P + A688G + T883R + F906A + Q912V + | | | |
| Y934G + Q956Y | | | |
| Y55M + A283D + S313D + E408D + Y579W + I602T + F638N + | 95% PUG | 672 | 321.6 |
| A651P + W719R + V756Y + N848D + A869V + R880K + | | | |
| N905D + K921R + Y934R | | | |
| F20P + S313D + E408D + Y579W + S636K + T697G + N848D + | 95% PUG | 672 | 325.9 |
| A937E + Q956Y | | | |
| F20N + Y55M + H311N + E408D + K512P + A559N + Y579W + | 95% PUG | 672 | 338.4 |
| A651P + T697G + N848D + K921R + Y934G | | | |
| Y55M + A283D + S313D + E408D + Y579W + I602T + F638N + | 95% PUG | 672 | 338.5 |
| A651P + W719R + V756Y + N848D + A869V + R880K + | | | |
| K921R + Y934R | | | |
| Y55M + A283D + I302D + S313D + E408D + Y579W + I602T + | 95% PUG | 672 | 342.9 |
| F638N + A651P + W719R + V756Y + N848D + A869V + R880K + | | | |
| K921R + Y934R | | | |
| Y55M + A283D + S313D + E408D + Y579W + I602T + F638N + | 95% PUG | 672 | 345.3 |
| A651P + W719R + V756Y + N848D + A869V + R880K + | | | |
| A885N + K921R + Y934R | | | |
| Y55M + A283D + S313D + E408D + Y579W + I602T + F638N + | 95% PUG | 672 | 345.9 |
| A651P + W719R + V756Y + N848D + A869V + R880K + | | | |
| N905D + K921R + Y934R | | | |
| Y55M + A283D + I302D + S313D + E408D + Y579W + I602T + | 95% PUG | 672 | 348.9 |
| F638N + A651P + W719R + V756Y + N848D + A869V + R880K + | | | |
| K921R + Y934R | | | |
| S313D + E408D + Y579W + I602T + F638N + A651P + T697G + | 95% PUG | 672 | 349.6 |
| N848D + R880K + K921R + Y934G | | | |
| F20P + I302D + S313D + E408D + D476R + Y579W + S636K + | 95% PUG | 672 | 351.2 |
| A651P + T697G + W719R + V756Y + N848D + V881T + | | | |
| T887K + F906A + A937E | | | |
| F20P + S313D + E408D + Y579W + S636K + T697G + N848D + | 95% PUG | 672 | 353.7 |
| A937E | | | |
| F20P + S313D + E408D + Y579W + S616G + S636K + T697G + | 95% PUG | 672 | 356.3 |
| W719R + T883V + N905D + A937E | | | |
| Y55M + A283D + S313D + E408D + Y579W + I602T + F638N + | 95% PUG | 672 | 364.7 |
| A651P + W719R + V756Y + N848D + A869V + R880K + | | | |
| N905D + K921R + Y934R | | | |
| F20P + S313D + E408D + Y579W + S636K + A688G + T697G + | 95% PUG | 672 | 365.7 |
| V881Q + N905D + K921R + A937E | | | |
| I302D + E408D + Q416S + Y579W + I602T + A651P + A688G + | 95% PUG | 672 | 367.3 |
| W719R + K720H + R880K + N905D + F1048W | | | |
| Y55M + A283D + I302D + S313D + E408D + Y579W + I602T + | 95% PUG | 672 | 367.5 |
| F638N + A651P + W719R + V756Y + N848D + A869V + R880K + | | | |
| K921R + Y934R | | | |
| F20P + S313D + E408D + Y579W + S636K + A688G + T697G + | 95% PUG | 672 | 375.4 |
| N905D + A937E | | | |
| F20P + I302D + S313D + E408D + D476R + Y579W + S636K + | 95% PUG | 672 | 376.0 |
| T697G + W719R + V756Y + V881Q + T887K + F906A + A937E | | | |
| Y55M + A283D + I302D + S313D + E408D + Y579W + I602T + | 95% PUG | 672 | 387.3 |
| F638N + A651P + W719R + V756Y + N848D + A869V + R880K + | | | |
| N905D + K921R + Y934R | | | |
| A283D + S313D + E408D + Y579W + I602T + F638N + A651P + | 95% PUG | 672 | 396.0 |
| T697G + K720H + V756Y + N848D + A869V + R880K + | | | |
| A885Q + K921R + Y934G | | | |
| F20P + I302D + S313D + E408D + D476R + Y579W + S636K + | 95% PUG | 672 | 406.2 |
| T697G + W719R + V756Y + V881Q + T887K + F906A + A937E | | | |
| F20P + S313D + E408D + Y579W + S636K + A688G + T697G + | 95% PUG | 672 | 409.6 |
| W719R + A885N + N905D + A937E | | | |
| Y55M + A283D + I302D + S313D + E408D + Y579W + I602T + | 95% PUG | 672 | 410.3 |
| F638N + A651P + W719R + V756Y + N848D + A869V + R880K + | | | |
| K921R + Y934R | | | |
| Y55M + A283D + S313D + E408D + Y579W + I602T + F638N + | 95% PUG | 168 | 411.7 |
| A651P + K720H + V756Y + N848D + A869V + R880K + | | | |
| K921R + Y934R | | | |
| F20P + I302D + S313D + E408D + Y579W + S636K + A688G + | 95% PUG | 672 | 412.1 |
| T697G + W719R + N905D + A937E | | | |
| N216D + I302D + H311N + E408D + Q416S + Y579W + I602T + | 95% PUG | 672 | 424.0 |
| A651P + A688G + W719R + A869V + R880K + V890R + N905D | | | |
| Y55M + A283D + I302D + S313D + E408D + Y579W + I602T + | 95% PUG | 672 | 426.7 |
| F638N + A651P + W719R + V756Y + N848D + A869V + R880K + | | | |
| A885N + N905D + K921R + Y934R | | | |
| F20P + N216Q + I302D + S313D + E408D + V567P + Y579W + | 95% PUG | 672 | 434.7 |
| S636K + A651P + T697G + W719R + N848D + T883R + | | | |
| N905D + A937E + Q956Y + F1048W | | | |
| F20P + K51Q + I302D + S313D + E408D + D476R + Q489P + | 95% PUG | 672 | 440.1 |
| AV756Y + S59N + Y579W + I602T + S636K + A651P + T697G + | | | |
| W719R + N848D + T883R + T887K + F906A + A937E | | | |
| F20P + I302D + S313D + E408D + D476R + Y579W + S636K + | 95% PUG | 672 | 442.1 |
| T697G + W719R + V756Y + V881Q + T887K + F906A + A937E | | | |
| N216Q + I302D + H311N + E408D + Q416S + Y579W + I602T + | 95% PUG | 672 | 450.7 |
| A651P + A688G + T697G + W719R + R880K + N905D + Q912V | | | |
| F20P + I302D + S313D + E408D + Y579W + S636K + A688G + | 95% PUG | 672 | 452.1 |
| T697G + W719R + A885N + N905D + A937E | | | |
| F20P + I302D + S313D + E408D + D476R + Q489P + A559N + | 95% PUG | 720 | 452.5 |
| Y579W + I602T + S636K + A651P + T697G + W719R + V756Y + | | | |
| N848D + T883R + T887K + F906A + A937E | | | |
| F20P + I302D + S313D + A346D + E408D + D476R + Q489P + | 95% PUG | 672 | 471.0 |
| Y579W + S636N + T697G + W719R + V756Y + A824D + | | | |
| N848D + V881Q + T887K + F906A + S928D + A937E + F1048W | | | |
| Y55M + A283D + S313D + E408D + Y579W + I602T + F638N + | 95% PUG | 672 | 471.7 |
| A651P + Y690F + T697G + W719R + V756Y + R880K + K921R + | | | |
| Y934G + A937E | | | |
| Y55M + A283D + I302D + S313D + A346D + E408D + Q489P + | 95% PUG | 720 | 472.1 |
| A559P + Y579W + I602T + V603P + F638N + A651P + T697G + | | | |
| W719R + K744H + A824D + N848D + R880K + V881T + F906A + | | | |
| Q912V + K921R + Y934G + A937E + K948R + Q956Y + F1048W | | | |
| F20P + I302D + S313D + E408D + Y579W + S636K + A688G + | 95% PUG | 672 | 472.2 |
| T697G + W719R + N905D + A937E | | | |
| N216D + S313D + E408D + D476R + Y579W + I602T + V603P + | 95% PUG | 720 | 492.4 |
| F638N + A651P + A688G + T697G + W719R + V756H + T825G + | | | |
| N833D + A869V + R880K + T887K + K921R + S928D + Y934G + | | | |
| N941S + K948R | | | |
| F20P + I302D + S313D + E408D + D476R + Y579W + S636K + | 95% PUG | 672 | 498.5 |
| A651P + T697G + W719R + V756Y + N848D + V881T + T887K + | | | |
| F906A + A937E | | | |
| F20P + I302D + S313D + E408D + D476R + Q489P + Y579W + | 95% PUG | 672 | 505.4 |
| S636N + T697G + W719R + V756Y + A824D + V881Q + T887K + | | | |
| F906A + A937E + T999R + A1037E | | | |
| F20P + I302D + S313D + A346D + E408D + D476R + Y579W + | 95% PUG | 672 | 507.2 |
| S636N + T697G + W719R + V756Y + A824D + N848D + V881Q + | | | |
| T887K + F906A + A937E + T999R + F1048W | | | |
| N216Q + S313D + E408D + D476R + Y579W + I602T + F638N + | 95% PUG | 672 | 521.0 |
| A651P + T697G + W719R + R880K + T887K + K921R + Y934G | | | |
| S17A + F20P + S313D + E408D + Y579W + I602T + A651P + | 95% PUG | 672 | 524.6 |
| T697G + W719R + N848D + T883R + F906A + Y934G + | | | |
| Q956Y + T999R | | | |
| SF20P + I302D + S313D + E408D + D476R + Y579W + I602T + | 95% PUG | 672 | 526.8 |
| S636K + T697G + W719R + V756Y + V881Q + T887K + F906A + | | | |
| A937E + T999R + F1048W | | | |
| F20P + I302D + S313D + A346D + E408D + D476R + Y579W + | 95% PUG | 672 | 531.0 |
| S636N + T697G + W719R + V756Y + N848D + T883R + F906A + | | | |
| S928D + A937E | | | |
| N216D + S313D + E408D + D476R + A564E + Y579W + I602T + | 95% PUG | 720 | 552.6 |
| F638N + A651P + Y690F + T697G + W719R + V756H + N833D + | | | |
| A869V + R880K + V881T + T887K + K921R + S928D + Y934G + | | | |
| T999R | | | |
| F20P + I302D + S313D + E408D + D476R + Y579W + S636K + | 95% PUG | 672 | 557.2 |
| T697G + W719R + V756Y + N848D + A869V + V881Q + | | | |
| T887K + N905D + F906A + Q912V + A937E + T999R + F1048W | | | |
| F20P + I302D + S313D + E408D + D476R + Y579W + S636K + | 95% PUG | 672 | 558.6 |
| T697G + W719R + V756Y + N848D + A869V + V881Q + | | | |
| T887K + F906A + Q912V + A937E + T999R + F1048W | | | |
| F20P + I302D + S313D + E408D + D476R + Q489P + Y579W + | 95% PUG | 672 | 565.9 |
| S636N + T697G + W719R + V756Y + A824D + N848D + V881Q + | | | |
| T887K + F906A + S928D + A937E | | | |
| Y55M + A283D + I302D + S313D + A386P + E408D + Q489P + | 95% PUG | 720 | 588.5 |
| A559P + Y579W + I602T + V603P + F638N + A651P + T697G + | | | |
| W719R + Q834E + N848D + R880K + V881T + T892P + F906A + | | | |
| Q912V + K921R + Y934G + A937E + F1048W | | | |
| F20P + I302D + S313D + E408D + Q489P + Y579W + I602T + | 95% PUG | 720 | 591.8 |
| S636N + T697G + W719R + V756Y + A824D + N848D + V881Q + | | | |
| T887K + N905D + F906A + S928D + A937E + T999R + A1037E + | | | |
| F1048W | | | |
| F20P + I302D + S313D + E408D + D476R + Y579W + I602T + | 95% PUG | 672 | 592.0 |
| V603P + S636K + T697G + W719R + V756Y + N848D + A869V + | | | |
| V881Q + T887K + F906A + Q912V + A937E + F1048W | | | |
| F20P + I302D + S313D + E408D + Y579W + I602T + S636K + | 95% PUG | 672 | 592.7 |
| A688G + T697G + W719R + V756Y + V881T + N905D + A937E | | | |
| F20P + I302D + S313D + E408D + Q416S + D476R + Q489P + | 95% PUG | 672 | 597.1 |
| Y579W + I602T + S636K + A651P + T697G + W719R + V756Y + | | | |
| A824D + N833D + N848D + T883R + T887K + F906A + A937E | | | |
| Y55M + A283D + I302D + S313D + E408D + A559P + Y579W + | 95% PUG | 720 | 604.2 |
| I602T + V603P + F638N + A651P + T697G + W719R + N833D + | | | |
| N848D + R880K + V881T + F906A + Q912V + K921R + Y934G + | | | |
| A937E + N941S + T999R | | | |
| F20P + A186P + I302D + S313D + E408D + D476R + Q489P + | | | |
| Y579W + A599S + I602T + S636K + A651P + T697G + W719R + | 95% PUG | 672 | 616.5 |
| V756Y + N848D + T883R + T887K + F906A + A937E | | | |
| Y55M + A283D + I302D + S313D + E408D + A559P + Y579W + | | | |
| I602T + V603P + F638N + A651P + T697G + W719R + N848D + | 95% PUG | 720 | 622.2 |
| A869V + R880K + V881T + F906A + Q912V + K921R + Y934G + | | | |
| A937E | | | |
| N216D + S313D + E408D + D476R + Y579W + I602T + V603P + | 95% PUG | 720 | 622.4 |
| F638N + A651P + A688G + T697G + W719R + V756H + R880K + | | | |
| T887K + K921R + S928D + Y934G + K948R | | | |
| F20P + A283D + I302D + S313D + E408D + Y579W + I602T + | 95% PUG | 672 | 624.4 |
| S636K + A651P + A688G + T697G + W719R + V756Y + T887K + | | | |
| N905D + A937E | | | |
| F20P + I302D + S313D + A346D + E408D + D476R + Y579W + | 95% PUG | 672 | 629.5 |
| I602T + T697G + W719R + N848D + V881Q + T887K + F906A + | | | |
| S928D + A937E + T999R | | | |
| F20P + K51Q + I302D + S313D + E408D + D476R + Q489P + | 95% PUG | 672 | 639.6 |
| Y579W + I602T + S636K + A651P + T697G + W719R + V756Y + | | | |
| N848D + T883R + T887K + F906A + A937E | | | |
| F20P + I302D + S313D + A346D + E408D + D476R + Q489P + | 95% PUG | 672 | 644.4 |
| Y579W + S636N + T697G + W719R + V756Y + A824D + | | | |
| N848D + V881Q + T887K + F906A + A937E + T999R | | | |
| F20P + I302D + S313D + E408D + D476R + Q489P + Y579W + | 95% PUG | 672 | 655.2 |
| I602T + S636N + T697G + W719R + V756Y + A824D + N848D + | | | |
| V881Q + T887K + N905D + F906A + A937E + T999R + A1037E + | | | |
| F1048W | | | |
| F20P + Q289E + I302D + S313D + E408D + D476R + Q489P + | 95% PUG | 720 | 658.0 |
| A559P + Y579W + I602T + S636N + T697G + W719R + V756Y + | | | |
| A824D + N848D + R880K + V881Q + T887K + N905D + F906A + | | | |
| S928D + Y934G + T999R + A1037E + F1048W | | | |
| F20P + K51Q + I302D + S313D + E408D + D476R + Q489P + | 95% PUG | 672 | 658.2 |
| Y579W + I602T + S636K + A651P + T697G + W719R + V756Y + | | | |
| A824D + N848D + T883R + T887K + F906A + S928D + A937E + | | | |
| A1037E | | | |
| N216D + S313D + A346D + E408D + D476R + Q489P + A559P + | 95% PUG | 720 | 662.8 |
| Y579W + I602T + F638N + A651P + A688G + T697G + W719R + | | | |
| V756H + R880K + T887K + K921R + S928D + Y934G | | | |
| F20P + I302D + S313D + A346D + E408D + D476R + Q489P + | 95% PUG | 672 | 663.4 |
| Y579W + I602T + T697G + W719R + V756Y + N848D + V881Q + | | | |
| T887K + F906A + A937E | | | |
| N216D + S313D + E408D + D476R + Q489P + A559P + Y579W + | 95% PUG | 720 | 679.1 |
| I602T + F638N + A651P + A688G + T697G + W719R + V756H + | | | |
| Q834E + R880K + T887K + T892P + K921R + S928D + Y934G | | | |
| F20P + I302D + S313D + E408D + D476R + Q489P + A559N + | 95% PUG | 672 | 693.2 |
| Y579W + I602T + S636K + A651P + T697G + W719R + V756Y + | | | |
| N848D + T883R + T887K + F906A + S928D + A937E | | | |
| F20P + I302D + S313D + E408D + D476R + Q489P + Y579W + | 95% PUG | 672 | 701.7 |
| I602T + S636K + A651P + T697G + W719R + V756Y + N848D + | | | |
| T883R + T887K + F906A + A937E | | | |
| F20P + I302D + S313D + E408D + Q416S + D476R + Q489P + | 95% PUG | 672 | 719.6 |
| A559N + Y579W + I602T + S636K + A651P + T697G + W719R + | | | |
| V756Y + N848D + T883R + T887K + F906A + A937E | | | |
| F20P + I302D + S313D + E408D + D476R + Y579W + I602T + | 95% PUG | 672 | 769.2 |
| S636N + T697G + W719R + V756Y + A824D + N848D + V881Q + | | | |
| T887K + F906A + S928D + A937E + T999R + F1048W | | | |

The HIFs of further purified variants tested are shown in Tables 13-16 below. HIFs in Tables 14-16 were calculated in the same way as in Table 13 except for a slightly increased incubation temperature for variants and controls (26-30 degrees celcius). All half-life values of the purified variants were calculated relative to the half-life of GH9 wild-type (mature parent endoclucanase with SEQ ID NO:2) incubated at the same detergent concentration and temperature measured as purified protein. HIF of GH9 wild-type in all tables is 1 (per definition).

TABLE 13
Purified variants of the mature parent GH9 endoglucanase (SEQ ID
NO: 2) with corresponding half-life improvement factors (HIF)
incubated at 25° C. relative to a parent endoglucanase,
e.g. an endoglucanase of SEQ ID NO: 2.

| Alteration as compared | PUG (detergent | Incubation | |
|---|---|---|---|
| to SEQ ID NO: 2 | concentration) | time (hrs) | HIF |
| K507R | 90% PUG | 16 | 1.1 |
| S17A | 90% PUG | 16 | 1.1 |
| S63F | 90% PUG | 16 | 1.1 |
| T694A | 90% PUG | 16 | 1.1 |
| F20Y + A448S + T781M | 90% PUG | 16 | 1.3 |
| Q834E | 90% PUG | 16 | 1.3 |
| V56M | 90% PUG | 16 | 1.3 |
| I602T | 90% PUG | 18 | 1.4 |
| A448E | 90% PUG | 16 | 1.4 |
| A448W | 90% PUG | 16 | 1.5 |
| F20G | 90% PUG | 16 | 1.5 |
| I403Y | 90% PUG | 16 | 1.5 |
| S538C | 90% PUG | 16 | 1.5 |
| E408N | 90% PUG | 16 | 2.2 |
| F20N | 90% PUG | 16 | 2.4 |
| K512P | 90% PUG | 16 | 2.5 |
| E408G | 90% PUG | 16 | 2.9 |
| P410G | 90% PUG | 16 | 2.9 |
| E408A | 90% PUG | 16 | 3.0 |
| F20P | 90% PUG | 16 | 3.3 |
| E408P | 90% PUG | 16 | 4.2 |
| E408S | 90% PUG | 16 | 5.4 |

TABLE 14
Purified variants of the mature parent GH9 endoglucanase (SEQ ID
NO: 2) with corresponding half-life improvement factors (HIF)
incubated at 26° C. relative to a parent endoglucanase,
e.g. an endoglucanase of SEQ ID NO: 2.

| Alteration as compared | PUG (detergent | Incubation | |
|---|---|---|---|
| to SEQ ID NO: 2 | concentration) | time (hrs) | HIF |
| I302H | 90% PUG | 17.25 | 1.1 |
| I387T | 90% PUG | 19.5 | 1.1 |
| K51H | 90% PUG | 19.5 | 1.1 |
| K754R | 90% PUG | 19.5 | 1.1 |
| K390Q | 90% PUG | 18 | 1.2 |
| S605T | 90% PUG | 18.5 | 1.3 |
| I602D | 90% PUG | 18.5 | 1.3 |
| K51Q | 90% PUG | 18 | 1.6 |
| Y55M | 90% PUG | 18 | 1.6 |
| K4515 | 90% PUG | 18 | 1.9 |
| Q4165 | 90% PUG | 18 | 2.2 |
| Q416D | 90% PUG | 18 | 2.5 |

TABLE 15
Purified variants of the mature parent GH9 endoglucanase (SEQ ID
NO: 2) with corresponding half-life improvement factors (HIF)
incubated at 28° C. relative to a parent endoglucanase,
e.g. an endoglucanase of SEQ ID NO: 2.

| Alteration as compared | PUG (detergent | Incubation | |
|---|---|---|---|
| to SEQ ID NO: 2 | concentration) | time (hrs) | HIF |
| K388R | 90% PUG | 5 | 1.3 |
| T711Y | 90% PUG | 5.5 | 1.8 |
| T711V | 90% PUG | 5.5 | 1.9 |

TABLE 16
Purified variants of the mature parent GH9 endoglucanase (SEQ ID
NO: 2) with corresponding half-life improvement factors (HIF)
incubated at 30° C. relative to a parent endoglucanase,
e.g. an endoglucanase of SEQ ID NO: 2.

| Alteration as compared | PUG (detergent | Incubation | |
|---|---|---|---|
| to SEQ ID NO: 2 | concentration) | time (hrs) | HIF |
| E408D + K4515 | 90% PUG | 16 | 26.1 |
| E408D + A448E | 90% PUG | 16 | 36.3 |
| E408D | 90% PUG | 138 | 53.3 |
| E408D + A448E | 90% PUG | 138 | 53.7 |
| E408D + K4515 | 90% PUG | 138 | 57.6 |
| Y579W + E408D | 90% PUG | 138 | 72.5 |

### Example 5

### Construction and Expression of Xanthan Lyase Variants

Xanthan lyase parent gene (i.e. SEQ ID NO:5) was PCR assembled into a linear cassette containing the promoter system on the upstream and cat selection maker on the downstream. To enable chromosomal integration of the cassette at the Pel locus of B. subtilis host by homologous recombination, >2 kb DNA sequence identical to the site of integration was included on both the sides of the cassette. Genomic DNA prepared from the strain containing xanthan lyase parent gene (SEQ ID NO:5) was used as template for generating the site-directed mutants. Mutagenic forward and reverse primers were used to generate an approximately 6 kb PCR fragment. This fragment was used as a megaprimer along with another forward primer to amplify >8 kb DNA fragment. This fragment contained the complete cassette (promoter system, xanthan lyase and cat gene along with homologous DNA sequence required for recombination at Pel locus) was used for transformation.

The triple promoter system used in the cassette has been described in WO99/43835 and it consists of promoters from Bacillus licheniformis alpha-amylase gene (amyL), Bacillus amyloliquefaciens alpha-amylase gene (amyQ), and the Bacillus thuringiensis cryIIIA promoter including the stabilizing sequence. Protease signal sequence from Bacillus clausii was included to export the protein out of the cells.

Generated variants of the mature parent xanthan lyase of SEQ ID NO:6 are shown in Tables 17, 18, and 19 below. The presence of the alteration was confirmed by sequencing.

TABLE 17
Generated variants of the mature parent xanthan lyase of
SEQ ID NO: 6

| Regions | Alteration |
|---|---|
| Region 1 (amino acids 154 to 176 of SEQ ID NO: 6): | Y155E |
| | A159P |
| Region 2 (amino acids 614 to 658 of SEQ ID NO: 6): | A624E |
| | A626G |
| | T631N |
| | T631E |
| | S635E |
| | S635T |
| | S635Q |
| | A645S |
| | T649V |
| | T649K |
| | T649R |
| | Q650G |
| | I656V |
| Region 3 (amino acids 731 to 803 of SEQ ID NO: 6): | G738L |
| | K745R |
| | F746L |
| | L748T |
| | P752R |
| | P752K |
| | G753E |
| | G753Q |
| | G753S |
| | S754E |
| | S754L |
| | S754Q |
| | S754R |
| | S757D |
| | S757P |
| | S757E |
| | P764V |
| | P764K |
| | A769D |
| | A769T |
| | A769R |
| | A769S |
| | A769E |
| | A769Q |
| | A769* |
| | A774V |
| | L775M |
| | L775Y |
| | L775A |
| | L775I |
| | L775S |
| | L775F |
| | L775Q |
| | D777K |
| | D777R |
| | P779V |
| | Y782I |
| | A785T |
| | N786K |
| | G789R |
| | K792W |
| | K792Y |
| | K792V |
| | K792A |
| | N796Q |
| | A799H |
| | V800P |
| | D801G |
| Region 4 (amino acids 807 to 846 of SEQ ID NO: 6): | K819R |
| | K819T |
| | K824R |
| | A843P |
| | D845E |
| Region 6 (amino acids 903 to 1004 of SEQ ID NO: 6): | T903A |
| | T903Q |
| | A911V |
| | A911M |
| | A911S |
| | A912T |
| | A912I |
| | A912Y |
| | T915Q |
| | T915S |
| | T915V |
| | T915A |
| | T919F |
| | T919G |
| | T919D |
| | T921R |
| | T921S |
| | T923H |
| | T923D |
| | T925Q |
| | T925D |
| | T925R |
| | T927K |
| | D928W |
| | Y930H |
| | Y930L |
| | Y930F |
| | A932P |
| | D933M |
| | G941E |
| | G941D |
| | A966P |
| | A967D |
| | N991D |
| | V998K |

TABLE 18
Generated variants of the mature parent xanthan lyase of SEQ ID NO: 6

| Regions | Alteration |
|---|---|
| Region 2 (amino acids 614 to 658 of SEQ ID NO: 6): | T631N |
| Region 3 (amino acids 731 to 803 of SEQ ID NO: 6): | A769D |
| | A769T |
| | L775A |
| | L775F |
| | L775I |
| | L775M |
| | L775Q |
| | L7755 |
| | L775Y |
| | P779V |
| | K792A |
| | K792V |
| | K792Y |
| | N796Q |
| | A799H |
| | D801G |
| Region 5 (amino acids 872 to 885 of SEQ ID NO: 6): | K875T |
| Region 6 (amino acids 903 to 1004 of SEQ ID NO: 6): | T903A |
| | T903Q |
| | A911M |
| | A911V |
| | A912I |
| | A912T |
| | A912Y |
| | T915S |
| | T915V |
| | T919D |
| | T919G |
| | T921R |
| | T921S |
| | T923D |
| | T923H |
| | T925D |
| | T925Q |
| | T925R |
| | T927K |
| | D928W |
| | Y930F |
| | Y930H |
| | Y930L |
| | D933M |
| | G941D |
| | V998K |

TABLE 19
Generated variants of the mature parent xanthan lyase
of SEQ ID NO: 6

| Regions | Alterations |
|---|---|
| Region 7 | K9R |
| (amino acids 1 to 153 of SEQ ID NO: 6) | N15T |
| | L46D |
| | A58L |
| | S66H |
| | Q89Y |
| | K95E |
| | S100D |
| | N106Y |
| | Q109[R, D, F, K, A] |
| Region 8 | K183[Q, R] |
| (amino acids 177 to 613 of SEQ ID NO: 6) | V188I |
| | A190Q |
| | A203P |
| | K204R |
| | A221P |
| | E229[N, S] |
| | I234V |
| | I238[W, L, M] |
| | I240W |
| | N242S |
| | G243V |
| | Y257W |
| | R258E |
| | K291R |
| | A293[G, P] |
| | K316R |
| | K320R |
| | L324Q |
| | K329R |
| | K333R |
| | L339M |
| | I341P |
| | V352I |
| | S354P |
| | K360R |
| | F377Y |
| | K400R |
| | F419Y |
| | D450P |
| | K451[E, R] |
| | A454V |
| | K481R |
| | A492L |
| | K567R |
| | G568A |
| | S578[K, R] |
| | S579[R, K] |
| Region 9 | T664K |
| (amino acids 659 to 730 of SEQ ID NO: 6) | N672D |
| Region 11 | K855R |
| (amino acids 847 to 871 of SEQ ID NO: 6) | |
| Region 12 | K887R |
| (amino acids 886 to 902 of SEQ ID NO: 6) | N892[Y, W, F] |

Bacillus organism containing a variant was inoculated in LB broth containing chloramphenicol (6 μg/mL) and grown overnight at 37° C. For expression of xanthan lyase variants, 2% of overnight culture was added to 300 mL of 10-R medium in 1000 mL baffled flask and grown at 30° C. for 96 h at 180 rpm.

10-R medium contained 33 g/L Soluble starch, 6 g/L (NH₄)₂HPO₄, 5 g/L Potato peptone, 1.2 g/L (MgSO₄ ×7 H₂O), 12 g/L KH₂PO₄, 5 g/L (Na₂HPO₄×2 H₂O), 18 mL/L of Trace metal solution, 1.8 g/L K₂SO₄ and 0.1 g/L (CaCl₂×2 H₂O and 0.5 mL/L SB2121 (anti-foam agent). Trace metal solution was made by mixing 0.49 g/L (MnSO₄×H₂O), 1.97 g/L (FeSO₄×7 H₂O), 0.1 g/L (CuSO₄×5 H₂O), 0.3 g/L ZnCl₂ and 19.6 g/L citric acid.

### Example 6

### Purification of Xanthan Lyase Variants

Prior to purification, Bacillus subtilis broth was clarified by centrifuging at 8000×g for 30 minutes at 10° C. followed by vacuum filtration using a combination of Seitz filter (K250) and WHATMAN glass filter GF/F grade in a Buchner funnel. Finally, the supernatant was filtered through 0.22μ Tangential flow filtration unit.

Xanthan lyase variants were purified using three-step automated tandem column chromatography. Macro-Prep Methyl HIC column was pre-equilibrated with 50 mM Tris, pH 8.0 containing 1 M (NH₄)₂SO₄ and 1 mM CaCl₂ buffer. During sample loading onto the column the clarified culture supernatant (250 mL) was diluted 1:1 in-line with 50 mM Tris, pH 8.0 containing 2 M (NH₄)₂SO₄ and1 mM CaCl₂ buffer to make the final concentration to 1 M. The unbound or weakly bound protein was washed with the equilibration buffer until the Absorbance at 280 nm comes below 0.1 AU. Elution was carried out using 50 mM Tris pH 8 containing 0.5 M (NH₄)₂SO₄ and 1 mM CaCl₂. Eluted protein peak was automatically loaded on MEP-Hypercel column pre-equilibrated with 50 mM Tris, pH 8 containing 0.5 M (NH₄)₂SO₄ and 1 mM CaCl₂. The unbound or weakly bound protein was washed with the equilibration buffer until the Absorbance at 280 nm comes below 0.1 AU. The column was washed again with 50 mM Tris, pH 8 containing 1 mM CaCl₂ to remove impurities. The Purified protein was eluted with 50 mM Na-acetate, pH 5 containing 1 mM CaCl₂. The eluted purified protein was automatically transferred to Sephadex G-25 column pre-equilibrated with 50 mM MOPS, pH 8 containing 1 mM CaCl₂ for desalting.

### Example 7

### Detergent Stability Assay

Reagents for the detergent stability assay were prepared as follows:

A stock of 1.0 M MOPS buffer was prepared by dissolving 209.26 g of 3-Morpholinopropanesulfonic acid in Milli Q water. pH was adjusted to 7.5 using NaOH and the final volume of buffer was made up to 1000 mL. This buffer stock was stored at 4° C. until use. A 50 mM working solution of MOPS buffer was prepared by adding 50 mL of 1.0 M stock to 950 mL of Milli Q water.

A substrate solution of 0.4% w/v xanthan gum was freshly prepared by dissolving 400 mg of xanthan gum in 100 mL of Milli Q water.

A stock solution mix containing 1.0 M Na₂CO₃, 0.17 M potassium sodium tartrate and 5 mM (Bi(NO₃)₃×5 H₂O) was prepared by dissolving 106.99 g of Na₂CO₃, 47.98 g of potassium sodium tartrate and 2.42 mg of (Bi(NO₃)₃×5 H₂O) in Milli Q water for a final volume of 1000 mL. This stock solution mix was filtered and stored at room temperature.

A PAHBAH reagent (1.5% PAHBAH) was freshly prepared by dissolving 1.5 g of p-hydroxybenzoic acid hydrazide (PAHBAH) in the stock solution mix.

Detergent Stability Assay:

A. Screening of Culture Supernatants of Variants

The in-detergent stability was determined by measuring the enzymatic activity present in culture supernatants of variants or wild-type controls after incubation with detergent (70%, final concentration) at 30° C.

Detergent stress was carried out by addition of 30 μL of culture supernatant and 70 pμL of a Persil Universal Gel detergent (100%) into wells of 96-well microtitre plates which were shaken for 15 min at 1000 rpm. Two identical plates were produced whereof one plate was incubated at 4° C. (unstressed plate) and the other plate was incubated at 30° C. (stressed plate) for 1 h. After incubation, samples from unstressed and stressed plates were diluted 50× with dilution buffer (50 mM MOPS, 5 mM CaCl₂, pH 7.5).

To measure the enzyme activity of diluted enzyme-detergent samples, reaction mixtures were prepared in 96-well PCR plates. 50 μL of diluted samples were mixed with 50 μL of freshly prepared substrate solution and incubated at 40° C. for 1 h.

After incubation, 75 μL of PAHBAH reagent was added to reaction mixture in the same PCR plate and incubated in a programmable thermal cycler (T-ROBOT) for 10 min at 90° C. followed by subsequent cooling at 10° C. Samples (25 μL) were transferred to a 384 well microtitre plate and the absorbance was measured at 405 nm using an Infinite M1000 reader (TECAN, Switzerland).

The residual activity (RA) for variants and wild-type controls was calculated as the percentage of enzymatic activity remaining after incubation at 30° C. relative to the enzymatic activity remaining after incubation at 4° C., i.e. according to the following formula after subtracting relevant background absorbance contributions:

Residual activity (RA)=100% * Abs405 (sample incubated at 30° C)/Abs405 (sample incubated at 4° C.).

The variants with higher detergent stability were picked with respect to the wild-types grown in the plates.

B. Screening of Purified Variants

The detergent stability of purified variants was determined by measuring the enzyme activity of the purified protein after incubation with detergent (90%, final concentration) at 30° C.

Purified variants were diluted to a concentration of 200 ppm using 50 mM MOPS buffer. For detergent treatment, 10 μL of diluted purified samples were mixed with 90 μL of Persil Universal Gel detergent (100%) into wells of 96-well microtitre plates which were shaken for 20 min at 1000 rpm. Two identical plates were produced whereof one plate was incubated at 4° C. (unstressed plate) and the other plate was incubated at 30° C. (stressed plate) for 1 h. After incubation, samples from unstressed and stressed plates were diluted 50× with dilution buffer (50 mM MOPS, 1 mM CaCl₂, pH 7.5).

Enzymatic activity analysis of unstressed and stressed samples was done as described in section A.

C. Calculating Half-Lives and Half-Life Improvement Factors (HIF)

Half-life (TV2 (in hours)) was calculated at a given detergent concentration and storage temperature for the wild-type controls and/or variants, as the degradation follows an exponential decay and the incubation time (hours) is known, i.e. according to the following formulas:

T½ (variant)=(Ln (0.5)/Ln (RA-variant/100))*Time

T½ (Wild-type)=(Ln (0.5)/Ln (RA-Wild-type/100))*Time

Where “RA” is the residual activity in percent as calculated above and “Time” is the incubation time in hours.

A half-life improvement factor (HIF) under a given set of storage conditions (detergent concentration and temperature) is calculated as HIF=T½ (variant)/T½ (Wild-type), where the Wild-type is incubated under the same storage conditions as the variant.

In cases where the difference in stability between wild-type and variants is too large to accurately assess half-life for both wild-type and variant using the same incubation time (see Table 24), the incubation time for wild-type and variant is different, e.g. 1 h for wild-type and up to 168 h for the most stable variants. Further, in order to determine the stability (half-life) within a shorter duration of incubation time for the more stable variants, e.g. <168 h, the incubation temperature for some variants in Table 24 was increased by 2-5 degrees Celsius.

The half-lives and calculated half-life improvement factor (HIF) values for culture supernatants of single mutation variants are provided in Table 22 below. Tables 23 and 24 show the half-life for purified variants having single, double or multiple mutations, as well as half-life improvement factor (HIF) values for the variants of Table 23. HIFs of all variants were calculated based on the wild-type xanthan lyase (SEQ ID NO:6) incubated at the same detergent contration and temperature (HIF of wild-type=1.0 in all tables, per definition).

The obtained HIF values for the purified variants are shown in Tables 20-24 below.

TABLE 20
Half-life improvement factors of purified variants:

| Regions | Alteration | HIF |
|---|---|---|
| — | Wild-type | 1 |
| Region 1 (amino acids 154 to 176 of SEQ ID NO: 6): | Y155E | 1.4 |
| Region 2 (amino acids 614 to 658 of SEQ ID NO: 6): | K620R | 1.5 |
| | Q650G | 4.8 |
| | T631N | 1.4 |
| | T649V | 1.2 |
| Region 3 (amino acids 731 to 803 of SEQ ID NO: 6): | K745R | 2.4 |
| | S757D | 3.4 |
| | G753E | 4.4 |
| | G753Q | 3.8 |
| | G753S | 2.5 |
| | S754E | 2 |
| | P752R | 1.4 |
| | S754L | 1.3 |
| | K792W | >5 |
| Region 4 (amino acids 807 to 846 of SEQ ID NO: 6): | K819R | 1.2 |
| | K824R | 1.2 |
| Region 6 (amino acids 903 to 1004 of SEQ ID NO: 6): | A966P | 1.5 |
| | N991D | 1.7 |

The obtained HIF values for culture supernatants of variants are shown in Table 21 below.

TABLE 21
Half-life improvement factors of culture supernatants of variants:

| Regions | Alteration | HIF |
|---|---|---|
| — | Wild-type | 1 |
| Region 2 (amino acids 614 to 658 of SEQ ID NO: 6): | T631N | 1.2 |
| Region 3 (amino acids 731 to 803 of SEQ ID NO: 6): | A769D | 3.2 |
| | A769T | 1.5 |
| | L775A | >5.0 |
| | L775F | 2.3 |
| | L775I | >5.0 |
| | L775M | >5.0 |
| | L775Q | 1.3 |
| | L775S | >5.0 |
| | L775Y | >5.0 |
| | P779V | >5.0 |
| | K792A | 1.9 |
| | K792V | 3.3 |
| | K792Y | >5.0 |
| | N796Q | 1.2 |
| | A799H | 1.3 |
| | D801G | 2.5 |
| Region 5 (amino acids 872 to 885 of SEQ ID NO: 6): | K875T | >5.0 |
| Region 6 (amino acids 903 to 1004 of SEQ ID NO: 6): | T903A | 1.3 |
| | T903Q | 1.2 |
| | A911M | 1.5 |
| | A911V | 2.1 |
| | A912I | 1.5 |
| | A912T | 1.6 |
| | A912Y | 1.4 |
| | T915S | 1.3 |
| | T915V | 1.2 |
| | T919D | 1.2 |
| | T919G | 1.3 |
| | T921R | 1.3 |
| | T921S | 1.2 |
| | T923D | 1.3 |
| | T923H | 2.3 |
| | T925D | 1.2 |
| | T925Q | 1.2 |
| | T925R | 1.2 |
| | T927K | 1.2 |
| | D928W | 2.3 |
| | Y930F | 1.2 |
| | Y930H | 1.2 |
| | Y930L | 1.2 |
| | D933M | 1.5 |
| | G941D | 1.2 |
| | V998K | 1.3 |

TABLE 22
Half-life and half-life improvement factors of culture
supernatants of variants

| | | Half- | |
|---|---|---|---|
| | | life | |
| Region | Mutation | (h) | HIF |
| — | Wild-type | 0.4 | 1.0 |
| Region 7 | A58L | 0.6 | 1.3 |
| (amino acids 1 to 153 of SEQ ID NO: 6) | Q89Y | 0.9 | 2.2 |
| | Q109A | 0.5 | 1.2 |
| | Q109D | 0.6 | 1.3 |
| | Q109F | 0.5 | 1.3 |
| | Q109K | 0.9 | 2.2 |
| Region 8 | K183Q | 0.5 | 1.2 |
| (amino acids 177 to 613 of SEQ ID NO: 6) | A190Q | 0.5 | 1.2 |
| | E229N | 0.8 | 1.9 |
| | I238L | 0.6 | 1.4 |
| | I238W | 0.7 | 1.6 |
| | K451E | 0.6 | 1.3 |
| | G568A | 1.1 | 2.5 |
| | S578K | 1.1 | 2.5 |
| | S579K | 0.8 | 2.0 |
| Region 9 | T664K | 0.5 | 1.1 |
| (amino acids 659 to 730 of SEQ ID NO: 6) | | | |
| Region 12 | N892F | 1.1 | 2.5 |
| (amino acids 886 to 902 of SEQ ID NO: 6) | N892W | 0.9 | 2.2 |

The obtained half-life and HIF values for purified variants tested at a 70% detergent concentration (30° C., incubation time 1 h) are shown in Table 23 below.

TABLE 23
Half-life and half-life improvement factors of purified variants

| | Half-life | |
|---|---|---|
| Mutations relative to SEQ ID NO: 6 | (h) | HIF |
| Wild-type | 0.2 | 1.0 |
| F377Y | 0.5 | 2.4 |
| S578R | 1.4 | 6.5 |
| S579R | 1.4 | 6.3 |
| N672D | 0.6 | 2.7 |
| N15T | 0.6 | 2.6 |
| V188I | 0.5 | 2.4 |
| I238M | 0.5 | 2.3 |
| Y257W | 0.5 | 2.3 |
| L324Q | 0.5 | 2.4 |
| S354P | 0.5 | 2.2 |
| K204R | 0.5 | 2.3 |
| K291R | 0.5 | 2.2 |
| K316R | 0.5 | 2.4 |
| K320R | 1.6 | 7.4 |
| K329R | 0.5 | 2.1 |
| K333R | 0.6 | 2.5 |
| K400R | 0.5 | 2.4 |
| K481R | 0.5 | 2.4 |
| K567R | 0.5 | 2.2 |
| Q109R | 1.8 | 8.0 |
| K95E | 0.5 | 2.4 |
| S100D | 0.5 | 2.2 |
| R258E | 0.5 | 2.4 |
| N15T + S579R | 1.5 | 6.7 |
| K9R | 0.5 | 2.0 |
| K183R | 0.5 | 2.2 |
| A293G + L324Q | 1.1 | 4.8 |
| N15T + K329R | 0.8 | 3.7 |
| L324Q + K329R | 0.8 | 3.4 |
| K316R + K329R | 0.8 | 3.6 |
| K333R + K855R | 1.1 | 4.8 |
| K329R + F377Y | 0.7 | 3.1 |
| A221P + K329R | 0.7 | 3.2 |
| N106Y + K329R | 0.7 | 3.2 |
| K360R + K855R | 0.8 | 3.6 |
| K360R + F377Y | 0.7 | 3.1 |
| K333R + K360R | 0.7 | 3.1 |
| L324Q + K329R | 0.7 | 3.1 |
| K329R + K360R | 0.6 | 2.8 |
| A293G + K316R | 0.7 | 3.3 |
| A293G + S579R | 1.3 | 5.9 |
| Q109R + R258E | 1.6 | 7.1 |
| Q109R + Y257W | 1.8 | 8.2 |
| Q109R + I238M | 2.1 | 9.5 |
| Q109R + K183R | 1.8 | 8.3 |
| S100D + K320R | 0.5 | 2.5 |
| S100D + Q109R | 1.6 | 7.1 |
| L46D + Q109R | 0.9 | 3.9 |
| N15T + Q109R | 1.3 | 5.9 |
| K451R + N672D | 0.6 | 2.5 |
| K451R + N892Y | 1.7 | 7.8 |
| K451R + S578R | 1.3 | 5.7 |
| K451R + S579R | 1.0 | 4.3 |
| K451R | 0.6 | 2.5 |
| V188I + L324Q | 0.7 | 3.2 |
| Q109R + A293P | 2.4 | 10.7 |
| Q109R + K400R | 1.8 | 8.2 |
| Q109R + K333R | 2.2 | 10.0 |

The obtained half-life values for purified variants tested at a 90% detergent concentration (temperature and incubation time as indicated) are shown in Table 24 below.

TABLE 24
Half-life values of purified variants

| | | Incubation | |
|---|---|---|---|
| | T | time | Half-life |
| Mutations relative to SEQ ID NO: 6 | (° C.) | (h) | (h) |
| Wild-type | 30 | 1 | 0.23 |
| V188I + K333R | 30 | 1 | 0.4 |
| V188I + L324Q | 30 | 1 | 0.4 |
| N672D + K855R | 30 | 1 | 0.4 |
| N242S + K329R + L339M + F377Y + S579R + N672D | 30 | 1 | 0.4 |
| I234V | 30 | 1 | 0.5 |
| I240W | 30 | 1 | 0.5 |
| I238M + L339M + F377Y + S579R + N672D | 30 | 1 | 0.5 |
| V352I | 30 | 1 | 0.5 |
| N242S + K291R + L339M + F377Y + S579R + N672D | 30 | 1 | 0.5 |
| K360R + K567R | 30 | 1 | 0.5 |
| K316R + S579R | 30 | 1 | 0.5 |
| N242S + L339M + F377Y + K567R + S579R + N672D | 30 | 1 | 0.5 |
| L46D + Q109R | 30 | 1 | 0.5 |
| K204R + N242S + L339M + F377Y + S579R + N672D | 30 | 1 | 0.5 |
| N242S + R258E + L339M + F377Y + S579R + N672D | 30 | 1 | 0.5 |
| N242S + L324Q + L339M + F377Y + S579R + N672D | 30 | 1 | 0.6 |
| E229S | 30 | 1 | 0.6 |
| D450P | 30 | 1 | 0.6 |
| A221P + N242S + L339M + F377Y + S579R + N672D | 30 | 1 | 0.6 |
| K291R + S579R | 30 | 1 | 0.6 |
| F419Y | 30 | 1 | 0.6 |
| S100D + Q109R | 30 | 1 | 0.6 |
| V188I + N672D | 30 | 1 | 0.6 |
| K887R | 30 | 1 | 0.7 |
| Q109R + K400R | 30 | 1 | 0.7 |
| K451R + S579R | 30 | 1 | 0.8 |
| V188I + S579R | 30 | 1 | 0.8 |
| L324Q + K360R | 30 | 1 | 0.8 |
| K291R + S578R | 30 | 1 | 0.8 |
| S100D + Q109R | 30 | 1 | 0.8 |
| A293G + S579R | 30 | 1 | 0.8 |
| Q109R + K333R | 30 | 1 | 0.8 |
| K204R + K320R | 30 | 1 | 0.8 |
| Q109R + K329R | 30 | 1 | 0.8 |
| Q109R + L324Q | 30 | 1 | 0.9 |
| S579R + K855R | 30 | 1 | 0.9 |
| K400R + K451R + N892Y | 30 | 1 | 0.9 |
| K291R + N672D | 30 | 1 | 0.9 |
| Q109R + A293P | 30 | 1 | 0.9 |
| K316R + K451R + N892Y | 30 | 1 | 0.9 |
| N15T + Q109R | 30 | 1 | 1.0 |
| Q109R + R258E | 30 | 1 | 1.0 |
| Q109R + K183R | 30 | 1 | 1.1 |
| K320R + K451R + N892Y | 30 | 3 | 1.1 |
| K451R + S578R | 30 | 1 | 1.2 |
| Q109R + Y257W | 30 | 1 | 1.2 |
| L46D + S579R + N892Y | 30 | 1 | 1.2 |
| Q109R + I238M | 30 | 1 | 1.2 |
| K451R + N892Y | 30 | 1 | 1.2 |
| K291R + K451R + N892Y | 30 | 1 | 1.3 |
| K9R + S579R + N892Y | 30 | 1 | 1.4 |
| K451R + N672D + N892Y | 30 | 3 | 1.4 |
| E229S + N672D | 30 | 3 | 1.4 |
| N892Y | 30 | 1 | 1.6 |
| K95E + S579R + N892Y | 30 | 1 | 1.7 |
| K183R + E229S | 30 | 3 | 1.7 |
| F377Y + S579R + N892Y | 30 | 1 | 1.7 |
| A454V + S579R | 30 | 3 | 1.7 |
| E229S + F377Y | 30 | 3 | 1.7 |
| S100D + S579R + N892Y | 30 | 1 | 1.7 |
| L324Q + K360R + S579R | 30 | 3 | 1.7 |
| Y257W + S579R + N892Y | 30 | 1 | 1.8 |
| L324Q + S579R + N892Y | 30 | 1 | 1.8 |
| E229S + L324Q | 30 | 3 | 1.8 |
| K316R + S579R + N892Y | 30 | 1 | 1.8 |
| K204R + E229S | 30 | 3 | 1.8 |
| E229S + K451R | 30 | 3 | 1.8 |
| N15T + S579R + N892Y | 30 | 1 | 1.9 |
| E229S + Y257W | 30 | 3 | 1.9 |
| E229S + I238M | 30 | 3 | 1.9 |
| S100D + E229S | 30 | 3 | 1.9 |
| E229S + K329R | 30 | 3 | 2.0 |
| K567R + S579R + N892Y | 30 | 1 | 2.0 |
| E229S + K291R | 30 | 3 | 2.0 |
| S66H + S578R | 30 | 3 | 2.0 |
| E229S + K316R | 30 | 3 | 2.0 |
| K9R + E229S | 30 | 3 | 2.0 |
| D450P + S578R | 30 | 3 | 2.0 |
| E229S + K320R | 30 | 3 | 2.0 |
| V188I + S579R + N892Y | 30 | 1 | 2.0 |
| A221P + E229S | 30 | 3 | 2.0 |
| R258E + K291R + S578R | 30 | 3 | 2.1 |
| Q109R + A454V | 30 | 3 | 2.1 |
| V188I + E229S | 30 | 3 | 2.1 |
| K329R + S579R + N892Y | 30 | 1 | 2.1 |
| L46D + K291R + S578R | 30 | 3 | 2.1 |
| I238M + G243V + K291R + L339M + S578R | 30 | 3 | 2.2 |
| Q109R + K451R + N892Y | 30 | 3 | 2.2 |
| A203P + K333R + S579R + N892Y | 30 | 1 | 2.2 |
| K451R + S578R + N892Y | 30 | 3 | 2.2 |
| K291R + S578R + N672D | 30 | 3 | 2.2 |
| K400R + S579R + N892Y | 30 | 1 | 2.2 |
| Q109R + F419Y | 30 | 3 | 2.2 |
| K291R + K320R + S578R | 30 | 3 | 2.3 |
| Q109R + D450P | 30 | 3 | 2.3 |
| K183R + K291R + S578R | 30 | 3 | 2.3 |
| K291R + S578R + N892Y | 30 | 3 | 2.3 |
| L324Q + S578R | 30 | 3 | 2.3 |
| Q109R + S578R + N892Y | 30 | 3 | 2.4 |
| K9R + K291R + S578R | 30 | 3 | 2.4 |
| K451R + S579R + N892Y | 30 | 1 | 2.4 |
| A221P + K291R + S578R | 30 | 3 | 2.5 |
| Q109R + K360R | 30 | 3 | 2.5 |
| A221P + S579R + N892Y | 30 | 1 | 2.5 |
| K291R + F377Y + S578R | 30 | 3 | 2.5 |
| Y257W + K291R + S578R | 30 | 3 | 2.6 |
| L324Q + K360R + S578R | 30 | 3 | 2.6 |
| K291R + K333R + S578R | 30 | 3 | 2.6 |
| K291R + K400R + S578R | 30 | 3 | 2.6 |
| K204R + S579R + N892Y | 30 | 1 | 2.6 |
| F419Y + S578R | 30 | 3 | 2.7 |
| I238M + K291R + S578R | 30 | 3 | 2.7 |
| S578R + K855R + N892Y | 30 | 3 | 2.7 |
| K291R + K567R + S578R | 30 | 3 | 2.8 |
| N15T + K291R + S578R | 30 | 3 | 2.8 |
| A454V + S578R | 30 | 3 | 2.8 |
| K291R + K451R + S578R | 30 | 3 | 2.8 |
| L324Q + S578R | 30 | 3 | 2.9 |
| K291R + K316R + S578R | 30 | 3 | 2.9 |
| K320R + S579R + N892Y | 30 | 1 | 3.0 |
| I341P + S578R | 30 | 3 | 3.0 |
| G568A + S578R | 30 | 3 | 3.0 |
| K360R + S578R | 30 | 3 | 3.1 |
| K204R + K291R + S578R | 30 | 3 | 3.1 |
| V188I + K291R + S578R | 30 | 3 | 3.2 |
| S100D + K291R + S578R | 30 | 3 | 3.3 |
| Q109R + K291R + S578R | 30 | 3 | 3.6 |
| K291R + L324Q + S578R | 30 | 3 | 3.6 |
| Q109R + S579R + N892Y | 30 | 1 | 3.7 |
| N106Y + S579R + N892Y | 30 | 1 | 4.3 |
| E229S + S579R | 30 | 3 | 5.2 |
| Q109R + E229S | 30 | 3 | 5.8 |
| N242S + L339M + F377Y + S579R + N672D + N892Y | 30 | 1 | 6.6 |
| Q109R + K887R | 30 | 3 | 7.5 |
| E229S + S578R | 30 | 3 | 8.1 |
| K204R + K291R + S578R | 30 | 20 | 9.7 |
| N15T + Q109R + K887R | 30 | 16 | 14 |
| S100D + K291R + K333R + S578R | 30 | 20 | 14 |
| Q109R + K183R + S579R + N892Y | 30 | 16 | 15 |
| N15T + Q109R + K291R + S578R | 30 | 20 | 16 |
| Q109R + K291R + S578K | 30 | 20 | 16 |
| E229S + L339M + S578R | 30 | 16 | 17 |
| E229S + S579R + N892Y | 30 | 20 | 17 |
| S100D + Q109R + S579R + N892Y | 30 | 16 | 17 |
| E229S + L324Q + S578R | 30 | 16 | 18 |
| S100D + Q109R + S578K + S579R + N892Y | 30 | 20 | 18 |
| Q109R + K291R + L324Q + S578R | 30 | 20 | 18 |
| Q109R + E229S + S578R | 30 | 16 | 18 |
| E229S + S579R + N672D | 30 | 20 | 18 |
| K183R + E229S + S578R | 30 | 16 | 18 |
| E229S + S578R + K855R | 30 | 16 | 19 |
| E229S + S578R + K887R | 30 | 16 | 19 |
| E229S + K400R + S578R | 30 | 16 | 20 |
| Q109R + K291R + S578R + N892Y | 30 | 20 | 21 |
| E229S + S579R + K855R | 30 | 20 | 16 |
| E229S + S579R | 30 | 20 | 14 |
| Q109R + K291R + K320R + S578R | 30 | 20 | 14 |
| K291R + K316R + S578R + K887R | 30 | 20 | 22 |
| Q109R + S578R + K887R | 30 | 20 | 23 |
| E229S + K291R + K360R + A492L + S578R + N892Y | 32 | 20 | 21 |
| K9R + E229S + S578R | 30 | 16 | 22 |
| E229S + S578R + N892Y | 32 | 20 | 23 |
| Q109R + K291R + S578R + K887R | 30 | 20 | 24 |
| E229S + K360R + S578R | 30 | 20 | 24 |
| E229S + S578K + N892Y | 32 | 20 | 24 |
| V188I + E229S + K291R + S578R | 30 | 20 | 25 |
| E229S + K360R + S578K | 32 | 20 | 26 |
| E229S + S578K | 32 | 20 | 26 |
| Q109R + E229S + K291R + S578R | 30 | 20 | 26 |
| Q109R + E229S + S578K | 35 | 70 | 38 |
| Q109R | 30 | 88 | 70 |
| L46D + Q109R + E229S + S578K | 35 | 166 | 82 |
| E229S + S578R + N892Y | 30 | 88 | 86 |
| Q109R | 30 | 88 | 91 |
| E229S + S578K | 30 | 88 | 118 |
| S100D + E229S + K360R + S578K | 30 | 168 | 137 |
| S100D + E229S + K291R + S578R | 30 | 168 | 139 |
| E229S + S578K + N892Y | 30 | 168 | 154 |
| S100D + E229S + S578K | 30 | 88 | 167 |
| E229S + S578K | 30 | 88 | 176 |
| E229S + S578K | 30 | 168 | 191 |
| E229S + A492L + S578K | 30 | 88 | 212 |
| Q109R + E229S + S578K | 30 | 88 | 250 |

### Example 8

### Half-Life of Xanthan Lyase Variants

Variants of the mature parent xanthan lyase of SEQ ID NO:6 were prepared and purified as described above in Examples 5 and 6. The in-detergent stability of the variants was determined as described in Example 7 by measuring the enzymatic activity present in either culture supernatants or purified samples of the variants after incubation with detergent. Incubation was performed using a 70% concentration of Persil Universal Gel detergent (PUG) at 30° C. for the culture supernatants, and a 70% or 90% concentration of PUG detergent at 30° C. for the purified variants, with a variant incubation time of 1 h for the culture supernatants and 1 h or 3 h for the purified variants.

The half-lives and calculated half-life improvement factor (HIF) values for culture supernatants are provided in Table 25 below. Table 26 shows the half-life and half-life improvement factors (HIF) for purified variants, where HIF for variants incubated with a 70% detergent concentration are calculated based on a wild-type half-life of 0.22 h and variants incubated with a 90% detergent concentration are calculated based on a wild-type half-life of 0.20 h.

TABLE 25
Half-life and half-life improvement factor (HIF) of culture supernatants of variants

| | | Half-life | |
|---|---|---|---|
| Region | Mutation | (h) | HIF |
| — | Wild-type | 0.42 | 1 |
| Region 1 (amino acids 154 to 176 of SEQ ID NO: 6) | A159P | 0.5 | 1.2 |
| Region 2 (amino acids 614 to 658 of SEQ ID NO: 6) | S635T | 0.6 | 1.4 |
| Region 3 | S754Q | 0.5 | 1.2 |
| (amino acids 731 to 803 of SEQ ID NO: 6) | S757E | 0.6 | 1.4 |
| | S757P | 0.6 | 1.4 |
| | A769R | 0.6 | 1.4 |
| | A769T | 1.1 | 2.6 |
| | L775F | 1.0 | 2.4 |
| | L775I | 1.1 | 2.6 |
| | L775Q | 0.5 | 1.2 |
| | L775S | 1.1 | 2.6 |
| | T778T | 0.6 | 1.4 |
| | Y782I | 0.7 | 1.6 |
| | N786K | 0.5 | 1.2 |
| | G789R | 0.5 | 1.2 |
| | K792A | 0.8 | 1.9 |
| | K792V | 1.1 | 2.6 |
| | K792Y | 1.1 | 2.6 |
| | N796Q | 0.5 | 1.2 |
| | A799H | 0.5 | 1.2 |
| Region 5 (amino acids 872 to 885 of SEQ ID NO: 6) | K875E | 0.6 | 1.4 |
| Region 6 (amino acids 903 to 1004 of SEQ ID NO: 6) | T903A | 0.5 | 1.2 |
| | T903Q | 0.5 | 1.2 |
| | A911M | 0.6 | 1.4 |
| | A911S | 0.6 | 1.4 |
| | A911V | 0.9 | 2.1 |
| | A912Y | 0.6 | 1.4 |
| | A912T | 0.7 | 1.6 |
| | T915Q | 0.6 | 1.4 |
| | T915S | 0.5 | 1.2 |
| | T919D | 0.5 | 1.2 |
| | T919F | 0.5 | 1.2 |
| | T921R | 0.6 | 1.4 |
| | T921S | 0.5 | 1.2 |
| | T923D | 0.5 | 1.2 |
| | T923H | 1.0 | 2.4 |
| | T925D | 0.5 | 1.2 |
| | T925Q | 0.5 | 1.2 |
| | T925R | 0.5 | 1.2 |
| | T927K | 0.5 | 1.2 |
| | D928W | 1.0 | 2.4 |
| | Y930F | 0.5 | 1.2 |
| | Y930H | 0.5 | 1.2 |
| | Y930L | 0.5 | 1.2 |
| | D933M | 0.6 | 1.4 |
| | G941E | 0.5 | 1.2 |

TABLE 26
Half-life and half-life improvement factor (HIF) of purified variants

| | | Incubation | | |
|---|---|---|---|---|
| | Detergent | time | Half-life | |
| Mutations | (%) | (h) | (h) | HIF |
| Wild-type | 70 | 1 | 0.22 | 1 |
| Wild-type | 90 | 1 | 0.2 | 1 |
| K620R | 70 | 1 | 0.6 | 2.6 |
| T631N | 70 | 1 | 0.6 | 2.5 |
| S635E | 90 | 1 | 0.7 | 3.3 |
| S757D | 70 | 1 | 0.6 | 2.9 |
| L775A | 90 | 3 | 1.6 | 8.1 |
| L775Y | 90 | 1 | 2.2 | 11 |
| L775M | 90 | 1 | 3.7 | 19 |
| P779V | 90 | 1 | 0.8 | 4.2 |
| D801G | 90 | 1 | 0.6 | 3.1 |
| A843P | 90 | 1 | 0.6 | 2.9 |
| K875T | 90 | 1 | 0.7 | 3.5 |
| T631N + K875T | 90 | 1 | 1.0 | 5.1 |
| S757D + D801G | 90 | 1 | 4.1 | 20 |
| S757D + K875T | 90 | 3 | 1.1 | 5.7 |
| K875T + N991D | 90 | 3 | 2.1 | 11 |

### Example 9

### Half-life of Xanthan Lyase Variants with Mutations in Chelator-Induced Instability Regions and Adjacent Regions

Variants of the mature parent xanthan lyase of SEQ ID NO:6 were prepared and purified as described above in Examples 5 and 6. For the purposes of this example, variants were produced having mutations in at least one chelator-induced instability region (regions 1, 2, 3, 4, 5, 6) and in at least one adjacent region (regions 7, 8, 9, 10, 11, 12, 13). The in-detergent stability of the variants was determined as described in Example 7 by measuring the enzymatic activity present in purified samples of the variants after incubation with detergent. Incubation was performed using a 70%, 90% or 95% concentration of Persil Universal Gel detergent (PUG), with incubation at a temperature of 30, 32, 35 or 37° C. and a variant incubation time ranging from 1 h to up to 840 h.

Half-lives were calculated as described above in Example 7. In cases where the difference in stability between wild-type and variants was too large to accurately assess half-life for both wild-type and variant using the same incubation time, the incubation time for wild-type and variant is different, e.g. 1 h for wild-type and up to 840 h for the most stable variants.

Further, in order to determine the stability (half-life) within a shorter duration of incubation time for the more stable variants, e.g. <168 h, the incubation temperature for some variants was increased by 2-7° C. For variants tested at a higher temperature (i.e. >30° C.), HIF values based on the wild-type could not be calculated as the half-life of the wild-type (order of magnitude of minutes) could not be determined accurately at these temperatures. Stability of the variants in the tables below is therefore reported in terms of half-life (in hours).

Tables 27-33 below show the half-life for the purified variants along with information on the test conditions (temperature, detergent concentration, incubation time) for each variant.

TABLE 27
Half-life of purified variants: Temperature (T) 30° C.,
detergent concentration 70%

| | | | Incuba- | |
|---|---|---|---|---|
| | | | tion | Half- |
| Mutations relative to | T | Detergent | time | life |
| SEQ ID NO: 6 | (° C.) | (%) | (h) | (h) |
| No mutations (Wild-type) | 30 | 70 | 1 | 0.22 |
| K620R + K855R | 30 | 70 | 1 | 1.0 |
| K329R + K745R | 30 | 70 | 1 | 0.8 |
| K360R + K745R | 30 | 70 | 1 | 0.7 |
| A293G + K567R + S579R + | 30 | 70 | 1 | 1.5 |
| K620R | | | | |
| S100D + N991D | 30 | 70 | 1 | 0.5 |

TABLE 28
Half-life of purified variants: Temperature (T) 30° C., detergent concentration 90%

| | | | Incubation | |
|---|---|---|---|---|
| | T | Detergent | time | Half-life |
| Mutations relative to SEQ ID NO: 6 | (° C.) | (%) | (h) | (h) |
| No mutations (Wild-type) | 30 | 90 | 1 | 0.20 |
| L339M + K451R + S579R + N672D + K745R + G899S | 30 | 90 | 1 | 0.4 |
| V188I + L339M + S579R + N672D + K745R + G899S | 30 | 90 | 1 | 0.5 |
| K291R + L339M + S579R + N672D + K745R + G899S | 30 | 90 | 1 | 0.5 |
| L339M + S579R + T631N + N672D + K745R + G899S | 30 | 90 | 1 | 0.5 |
| N242S + L339M + F377Y + S579R + T631N + N672D | 30 | 90 | 1 | 0.5 |
| L339M + F377Y + S579R + N672D + K745R + G899S | 30 | 90 | 1 | 0.5 |
| F377Y + T631N + K819R + N892Y | 30 | 90 | 1 | 0.5 |
| A221P + L339M + S579R + N672D + K745R + G899S | 30 | 90 | 1 | 0.6 |
| K316R + L339M + S579R + N672D + K745R + G899S | 30 | 90 | 1 | 0.6 |
| I238M + L339M + S579R + N672D + K745R + G899S | 30 | 90 | 1 | 0.6 |
| S579R + N991D | 30 | 90 | 1 | 0.6 |
| K9R + T631N + K819R + N892Y | 30 | 90 | 1 | 0.6 |
| S578R + K819R | 30 | 90 | 1 | 0.6 |
| S579R + K819R | 30 | 90 | 1 | 0.6 |
| N242S + L339M + F377Y + S579R + N672D + T727P + N991D | 30 | 90 | 1 | 0.6 |
| N242S + L339M + F377Y + S579R + N672D + S757D | 30 | 90 | 1 | 0.7 |
| S578R + N991D | 30 | 90 | 1 | 0.8 |
| A293G + K567R + S579R + K620R | 30 | 90 | 1 | 0.8 |
| Q109R + L339M + S579R + N672D + K745R + G899S | 30 | 90 | 1 | 0.9 |
| S579R + I722F + T727P + K819R + N892Y | 30 | 90 | 1 | 0.9 |
| N892Y + N991D | 30 | 90 | 1 | 0.9 |
| N15T + N892Y + N991D | 30 | 90 | 1 | 1.0 |
| T631N + K819R + N892Y | 30 | 90 | 1 | 1.1 |
| K451R + K620R + N892Y | 30 | 90 | 1 | 1.1 |
| N672D + K819R + N892Y | 30 | 90 | 3 | 1.2 |
| S579R + T631N + A645S + N892Y | 30 | 90 | 1 | 1.3 |
| K819R + N892Y | 30 | 90 | 1 | 1.3 |
| S579R + K620R + N892Y | 30 | 90 | 1 | 1.3 |
| N672D + K875T | 30 | 90 | 3 | 1.3 |
| K316R + S578R + K819R | 30 | 90 | 3 | 1.4 |
| N15T + T631N + K819R + N892Y | 30 | 90 | 3 | 1.4 |
| K183R + T631N + K819R + N892Y | 30 | 90 | 3 | 1.4 |
| S579R + A843P | 30 | 90 | 3 | 1.6 |
| S579R + T631N + K819R + N892Y | 30 | 90 | 3 | 1.6 |
| E229S + S757D | 30 | 90 | 3 | 1.7 |
| R258E + S578R + K819R | 30 | 90 | 3 | 1.7 |
| S579R + S635T | 30 | 90 | 3 | 1.8 |
| E229S + K620R | 30 | 90 | 3 | 1.8 |
| S579R + K875T | 30 | 90 | 3 | 1.8 |
| S578R + N672D + K819R | 30 | 90 | 3 | 1.8 |
| S579R + D801G | 30 | 90 | 3 | 1.8 |
| S579R + T727P + N892Y + N991D | 30 | 90 | 1 | 1.9 |
| S579R + N892Y + N991D | 30 | 90 | 3 | 1.9 |
| A221P + S578R + K819R | 30 | 90 | 3 | 1.9 |
| K451R + T631N + N892Y | 30 | 90 | 1 | 1.9 |
| N672D + D801G | 30 | 90 | 1 | 2.0 |
| K291R + S578R + K819R | 30 | 90 | 3 | 2.0 |
| I238M + S578R + K819R | 30 | 90 | 3 | 2.1 |
| S578R + D801G + K819R | 30 | 90 | 3 | 2.1 |
| S578R + D801G | 30 | 90 | 3 | 2.1 |
| S578R + S757D + K819R | 30 | 90 | 3 | 2.2 |
| Q109R + K819R + N892Y | 30 | 90 | 3 | 2.2 |
| V188I + S578R + K819R | 30 | 90 | 3 | 2.2 |
| K9R + S578R + K819R | 30 | 90 | 3 | 2.2 |
| L324Q + S578R + K819R | 30 | 90 | 3 | 2.3 |
| K95E + S578R + K819R | 30 | 90 | 3 | 2.3 |
| S578R + K819R + N892Y + N991D | 30 | 90 | 3 | 2.3 |
| S578R + T631N + K819R + N892Y | 30 | 90 | 3 | 2.3 |
| S100D + S578R + N892Y + A967D | 30 | 90 | 3 | 2.3 |
| V188I + S578R + N892Y + A967D | 30 | 90 | 3 | 2.3 |
| K291R + S578R + K875T | 30 | 90 | 3 | 2.3 |
| K567R + S578R + K819R | 30 | 90 | 3 | 2.4 |
| K316R + S578R + K819R | 30 | 90 | 3 | 2.4 |
| Q109R + T631N + K819R + N892Y | 30 | 90 | 3 | 2.4 |
| Q109R + A843P | 30 | 90 | 3 | 2.4 |
| K204R + S578R + K819R | 30 | 90 | 3 | 2.4 |
| S578R + K875T | 30 | 90 | 3 | 2.4 |
| Q109R + D801G | 30 | 90 | 3 | 2.4 |
| K291R + S578R + N991D | 30 | 90 | 3 | 2.4 |
| N106Y + S578R + K819R | 30 | 90 | 3 | 2.5 |
| Q109R + S635T | 30 | 90 | 3 | 2.5 |
| K291R + S578R + D801G | 30 | 90 | 3 | 2.5 |
| S578R + K819R + N892Y | 30 | 90 | 3 | 2.5 |
| K183R + S578R + K819R | 30 | 90 | 3 | 2.5 |
| S578R + K620R + K819R | 30 | 90 | 3 | 2.6 |
| K291R + S578R + K620R | 30 | 90 | 3 | 2.6 |
| S578R + S635T | 30 | 90 | 3 | 2.6 |
| S578R + A843P | 30 | 90 | 3 | 2.7 |
| K291R + S578R + T631N | 30 | 90 | 3 | 2.7 |
| S578R + K819R + K875T | 30 | 90 | 3 | 2.7 |
| N15T + S578R + K819R | 30 | 90 | 3 | 2.8 |
| L339M + S578R + K819R | 30 | 90 | 3 | 2.9 |
| K291R + S578R + S757D | 30 | 90 | 3 | 2.9 |
| Q109R + K875T | 30 | 90 | 3 | 2.9 |
| F377Y + S579R + K745R | 30 | 90 | 3 | 2.9 |
| Q109R + S578R + K819R | 30 | 90 | 3 | 3.7 |
| L339M + S579R + N672D + P726Q + T727P + K745R + A785T + | 30 | 90 | 1 | 4.2 |
| N892Y + G899S | | | | |
| S100D + R317K + S578R + K620R | 30 | 90 | 1 | 4.5 |
| S579R + S757D + N892Y | 30 | 90 | 1 | 4.7 |
| K875T + N892Y | 30 | 90 | 3 | 9.3 |
| N106Y + S578R + K819R + K875T | 30 | 90 | 16 | 9.7 |
| N15T + S578R + L775A + K819R | 30 | 90 | 16 | 11 |
| Q109R + N672D + K875T | 30 | 90 | 16 | 11 |
| Q109R + S757D + K875T | 30 | 90 | 16 | 11 |
| K183R + S578R + L775A + K819R | 30 | 90 | 16 | 11 |
| N106Y + S578R + L775A + K819R | 30 | 90 | 16 | 11 |
| N15T + F377Y + S578R + K819R | 30 | 90 | 16 | 12 |
| Q109R + D801G + K819R + N892Y | 30 | 90 | 16 | 12 |
| K183R + S578R + K819R + K875T | 30 | 90 | 16 | 12 |
| Q109R + D801G + K875T | 30 | 90 | 16 | 12 |
| Q109R + K819R + K875T + N892Y | 30 | 90 | 16 | 12 |
| Q109R + P779V + K819R + N892Y | 30 | 90 | 16 | 12 |
| Q109R + P779V + K875T | 30 | 90 | 16 | 13 |
| V188I + K291R + S578R + L775A | 30 | 90 | 20 | 13 |
| I341P + S578R + L775A | 30 | 90 | 16 | 13 |
| Q109R + F377Y + K875T | 30 | 90 | 16 | 13 |
| Q109R + S578R + K875T | 30 | 90 | 16 | 13 |
| Q109R + S579R + K819R + N892Y | 30 | 90 | 16 | 13 |
| K291R + L324Q + S578R + K620R | 30 | 90 | 20 | 13 |
| Q109R + T631N + K875T | 30 | 90 | 16 | 13 |
| Q109R + S578R + L775A + K819R | 30 | 90 | 16 | 13 |
| I341P + S578R + L775M | 30 | 90 | 16 | 13 |
| I341P + S578R + T631N | 30 | 90 | 16 | 14 |
| Q109R + K875T + N991D | 30 | 90 | 16 | 14 |
| Q109R + K620R + K875T | 30 | 90 | 16 | 14 |
| K320R + S578R + L775A + K819R | 30 | 90 | 16 | 14 |
| Q109R + L775M + K875T | 30 | 90 | 16 | 14 |
| Q109R + K875T + N892Y | 30 | 90 | 16 | 15 |
| E229S + S578R + D801G | 30 | 90 | 16 | 15 |
| Q109R + S578R + L775A + K875T | 30 | 90 | 20 | 15 |
| Q109R + L775M + K819R + N892Y | 30 | 90 | 16 | 15 |
| I341P + S578R + K875T | 30 | 90 | 16 | 16 |
| Q109R + L775A + K819R + N892Y | 30 | 90 | 16 | 16 |
| Q109R + S579R + D801G + K875T | 30 | 90 | 20 | 16 |
| E229S + S578R + P779V | 30 | 90 | 16 | 16 |
| Q109R + L775A + K875T | 30 | 90 | 16 | 16 |
| Q109R + S578K + K819R + K875T + N892Y | 30 | 90 | 20 | 17 |
| E229S + S578R + K875T | 30 | 90 | 16 | 17 |
| Q109R + L775A + P779V + K792Y + K819R + N892Y | 30 | 90 | 20 | 18 |
| E229S + S579R + L775A | 30 | 90 | 20 | 18 |
| E229S + S579R + D928W | 30 | 90 | 20 | 18 |
| E229S + S578R + L775A | 30 | 90 | 16 | 18 |
| E229S + S578R + K819R | 30 | 90 | 16 | 18 |
| V188I + S578R + L775A + N892Y + A967D | 30 | 90 | 16 | 19 |
| Q109R + E229S + K819R + K875T + N892Y | 30 | 90 | 20 | 20 |
| Q109R + L775A + P779V + K875T + N892Y | 30 | 90 | 20 | 20 |
| Q109R + D801G + K819R + K875T + N892Y | 30 | 90 | 20 | 20 |
| E229S + S578R + N991D | 30 | 90 | 16 | 20 |
| L339M + S578R + K819R | 30 | 90 | 20 | 21 |
| E229S + S579R + K875T | 30 | 90 | 20 | 21 |
| Q109R + N892Y + N991D | 30 | 90 | 20 | 10 |
| K320R + S578R + K819R + K875T | 30 | 90 | 20 | 9 |
| K183R + S578R + K819R + N892Y | 30 | 90 | 20 | 9 |
| Q109R + S578R + K819R + K875T | 30 | 90 | 20 | 14 |
| E229S + S579R + A843P | 30 | 90 | 20 | 17 |
| Q109R + S579R + A843P | 30 | 90 | 20 | 12 |
| S578R + D801G + A843P | 30 | 90 | 20 | 10 |
| Q109R + S578K + K875T + N892Y | 30 | 90 | 20 | 22 |
| Q109R + L775A + P779V + K792Y + K819R + K875T + N892Y | 30 | 90 | 20 | 22 |
| E229S + S578R + K620R | 30 | 90 | 16 | 26 |
| S578R + K620R + A769# | 30 | 90 | 3 | 26 |
| Q109R + L775A + D801G + K875T | 30 | 90 | 20 | 26 |
| E229S + S578R + T923H | 30 | 90 | 16 | 26 |
| Q109R + L775A + K792Y + K819R + N892Y | 30 | 90 | 20 | 27 |
| Q109R + L775M + K875T + N892Y | 30 | 90 | 20 | 27 |
| Q109R + S578K + L775A + K819R + N892Y | 30 | 90 | 20 | 27 |
| Q109R + A769T + L775A + K792Y + K819R + N892Y | 30 | 90 | 20 | 29 |
| Q109R + P779V + K792Y + K875T + N892Y | 30 | 90 | 20 | 29 |
| L775A + K875T + N892Y | 30 | 90 | 88 | 33 |
| Q109R + L775A + P779V + K792Y + K875T + N892Y | 30 | 90 | 20 | 33 |
| S578K + L775A + K875T + N892Y + A911V | 30 | 90 | 90 | 38 |
| Q109R + L775A + K792Y + K875T | 30 | 90 | 20 | 43 |
| S578K + P752K + G753E + L775A + K875T + N892Y | 30 | 90 | 90 | 46 |
| Q109R + L775A + K792Y + K875T + N892Y | 30 | 90 | 20 | 47 |
| S578K + L775A + K875T + N892Y + A912T | 30 | 90 | 90 | 49 |
| S578K + P752R + G753E + L775A + K875T + N892Y | 30 | 90 | 90 | 49 |
| S578K + P752R + G753E + S754E + L775A + K875T + N892Y | 30 | 90 | 90 | 51 |
| Q109R + L775A + K875T + N892Y | 30 | 90 | 88 | 54 |
| S578K + G753E + S754E + L775A + K875T + N892Y | 30 | 90 | 167 | 61 |
| S578K + P752K + G753E + S754E + L775A + K875T + N892Y | 30 | 90 | 167 | 62 |
| Q109R + E229V + L775A + D801G + K875T | 30 | 90 | 88 | 67 |
| S578K + P752R + S754E + L775A + K875T + N892Y | 30 | 90 | 167 | 67 |
| S578K + A769D + L775A + K875T + N892Y | 30 | 90 | 90 | 68 |
| S578K + L775A + D801G + K875T + N892Y | 30 | 90 | 90 | 69 |
| Q109R + A769T + L775A + K875T + N892Y | 30 | 90 | 168 | 71 |
| Q109R + S754E + A769T + L775A + K875T + N892Y | 30 | 90 | 88 | 72 |
| S578K + G753E + L775A + K875T + N892Y | 30 | 90 | 167 | 73 |
| Q109R + L775A + K875T + N892Y | 30 | 90 | 168 | 75 |
| Q109R + A769T + L775A + K792Y + K875T + N892Y | 30 | 90 | 168 | 78 |
| E229S + G753E + S754E + L775A + K875T + N892Y | 30 | 90 | 167 | 79 |
| E229S + P752R + G753E + L775A + K875T + N892Y | 30 | 90 | 167 | 80 |
| E229S + P752K + G753E + L775A + K875T + N892Y | 30 | 90 | 167 | 81 |
| S100D + L775A + D801G + K875T + N892Y | 30 | 90 | 167 | 81 |
| Q109R + A769T + L775A + K875T + N892Y | 30 | 90 | 168 | 82 |
| E229S + P752R + G753E + S754E + L775A + K875T + N892Y | 30 | 90 | 167 | 82 |
| S754E + L775A + D801G + K875T + N892Y | 30 | 90 | 167 | 86 |
| E229S + S578R + G753E + N892Y | 30 | 90 | 88 | 86 |
| L775A + D801G + K875T + N892Y | 30 | 90 | 168 | 86 |
| Q109R + S578K + L775A + D801G + K875T | 30 | 90 | 88 | 86 |
| E229S + S578R + L775A + N892Y | 30 | 90 | 88 | 87 |
| E229S + G753E + L775A + K875T + N892Y | 30 | 90 | 167 | 89 |
| Q109R + E229S + A769T + L775A + K875T + N892Y | 30 | 90 | 88 | 95 |
| Q109R + L775A + P779V + K792Y + D801G + K819R + | 30 | 90 | 88 | 96 |
| K875T + N892Y | | | | |
| Q109R + G753E + S754E + A769T + L775A + K875T + N892Y | 30 | 90 | 168 | 102 |
| Q109R + P752R + G753E + S754E + A769T + L775A + | 30 | 90 | 168 | 102 |
| K875T + N892Y | | | | |
| E229S + P752K + G753E + S754E + L775A + K875T + N892Y | 30 | 90 | 167 | 103 |
| E229S + S578R + G753E + A769D + K792Y + N892Y | 30 | 90 | 168 | 106 |
| P752R + S754E + L775A + D801G + K875T + N892Y | 30 | 90 | 167 | 109 |
| S100D + E229S + S578R + N892Y + A912T | 30 | 90 | 168 | 109 |
| E229S + S578R + L775A + P779V + K792Y + N892Y | 30 | 90 | 168 | 110 |
| E229S + S578R + P752K + S754E + K792Y + N892Y + A912T | 30 | 90 | 168 | 112 |
| E229S + S578R + P752R + G753E + K792Y + N892Y + A912T | 30 | 90 | 168 | 116 |
| E229S + L775A + D801G + K875T + N892Y | 30 | 90 | 167 | 116 |
| Q109R + P752K + G753E + A769T + L775A + K875T + N892Y | 30 | 90 | 88 | 117 |
| E229S + S578K + A769D + K792Y | 30 | 90 | 88 | 119 |
| Q109R + P752K + G753E + L775A + D801G + K875T | 30 | 90 | 88 | 120 |
| Q109R + G753E + A769T + L775A + K875T + N892Y | 30 | 90 | 88 | 120 |
| E229S + S578R + K792Y + D801G + N892Y | 30 | 90 | 168 | 120 |
| E229S + S578K + L775A + P779V + K792Y | 30 | 90 | 88 | 122 |
| E229S + S578R + A769D + P779V + K792Y + N892Y | 30 | 90 | 168 | 122 |
| E229S + A492L + S578R + N892Y + A912T | 30 | 90 | 168 | 122 |
| E229S + S578R + A7695 + K792Y + N892Y | 30 | 90 | 168 | 123 |
| E229S + A769D + L775A + K875T + N892Y | 30 | 90 | 167 | 125 |
| Q109R + E229S + A769T + L775A + K792Y + K875T + N892Y | 30 | 90 | 168 | 125 |
| G753E + L775A + D801G + K875T + N892Y | 30 | 90 | 168 | 127 |
| E229S + F419Y + S578K + G753E | 30 | 90 | 168 | 127 |
| E229S + S578R + A769D + L775A + K792Y + N892Y + A912T | 30 | 90 | 168 | 127 |
| Q109R + E229S + P752K + G753E + A769T + L775A + | 30 | 90 | 168 | 128 |
| K875T + N892Y | | | | |
| Q109R + E229S + P752K + S754E + A769T + L775A + | 30 | 90 | 168 | 130 |
| K875T + N892Y | | | | |
| Q109R + E229S + G753E + A769T + L775A + K875T + N892Y | 30 | 90 | 168 | 130 |
| Q109R + E229S + P752R + G753E + S754E + A769T + | 30 | 90 | 168 | 131 |
| L775A + K875T + N892Y | | | | |
| E229S + S578K + D801G | 30 | 90 | 88 | 133 |
| Q109R + P752K + G753E + S754E + A769T + L775A + | 30 | 90 | 88 | 133 |
| K875T + N892Y | | | | |
| E229S + S578K + G753E + A843P | 30 | 90 | 168 | 134 |
| E229S + S578R + L775A + K792Y + N892Y + A912T | 30 | 90 | 168 | 137 |
| E229S + S578K + T631N + G753E | 30 | 90 | 168 | 137 |
| E229S + S578K + P752R + G753E | 30 | 90 | 88 | 139 |
| Q109R + P752R + G753E + A769T + L775A + K875T + N892Y | 30 | 90 | 88 | 139 |
| E229S + A492L + S578K + G753E + D801G | 30 | 90 | 168 | 140 |
| Q109R + E229S + G753E + S754E + A769T + L775A + | 30 | 90 | 168 | 141 |
| K875T + N892Y | | | | |
| E229S + S578K + K875T | 30 | 90 | 88 | 143 |
| E229S + S578R + G753E + A769D + L775A + N892Y | 30 | 90 | 167 | 143 |
| G753E + L775A + D801G + K875T + N892Y | 30 | 90 | 167 | 144 |
| E229S + S578K + G753E + N892Y | 30 | 90 | 168 | 144 |
| E229N + S578K + A769D + L775A + K875T + N892Y | 30 | 90 | 168 | 145 |
| E229S + S578K + G753E + L775A + P779V | 30 | 90 | 168 | 145 |
| Q109R + E229S + S578K + A769T + L775A + K875T + N892Y | 30 | 90 | 168 | 146 |
| E229S + S578R + P752K + K792Y + N892Y + A912T | 30 | 90 | 168 | 149 |
| E229S + K360R + S578K + P752K + G753E + S754E | 30 | 90 | 168 | 149 |
| E229S + A492L + S578K + G753E | 30 | 90 | 168 | 150 |
| E229S + S578K + K792Y + N892Y | 30 | 90 | 168 | 151 |
| E229S + S578K + P779V | 30 | 90 | 88 | 151 |
| E229S + S578K + G753E | 30 | 90 | 88 | 153 |
| E229S + K360R + S578K + A769D + L775A + K792Y | 30 | 90 | 168 | 154 |
| Q109R + E229S + T631N + G753E + S754E + A769T + L775A + | 30 | 90 | 168 | 156 |
| K875T + N892Y | | | | |
| Q109R + E229S + A769D + L775A + K875T + N892Y | 30 | 90 | 168 | 156 |
| G753E + L775A + D801G + K875T + N892Y + A912T | 30 | 90 | 168 | 157 |
| Q109R + E229S + K567R + G753E + S754E + A769T + L775A + | 30 | 90 | 168 | 158 |
| K875T + N892Y | | | | |
| P752R + G753E + L775A + D801G + K875T + N892Y | 30 | 90 | 167 | 158 |
| E229S + S578K + T631N + P752K + G753E + S754E | 30 | 90 | 168 | 159 |
| E229S + A492L + S578K + G753E + K1016T | 30 | 90 | 168 | 159 |
| E229S + S578K + T631N + G753E + S754E | 30 | 90 | 168 | 159 |
| E229S + S578K + P752R | 30 | 90 | 88 | 160 |
| E229S + S578R + G753E + A769D + P779V + N892Y | 30 | 90 | 168 | 160 |
| E229S + S578K + G753E + N1008D | 30 | 90 | 168 | 162 |
| P752K + G753E + L775A + D801G + K875T + N892Y | 30 | 90 | 167 | 162 |
| E229S + S578K + G753E + A912T | 30 | 90 | 168 | 163 |
| E229S + S578K + G753E + S754E | 30 | 90 | 88 | 165 |
| E229S + S578K + P752R + S754E | 30 | 90 | 88 | 165 |
| E229S + A492L + S578K + G753E + S754E | 30 | 90 | 168 | 167 |
| E229S + S578K + A769D + P779V | 30 | 90 | 88 | 168 |
| A769D + L775A + D801G + K875T + N892Y | 30 | 90 | 167 | 172 |
| Q109R + E229S + G753E + S754E + A769T + L775A + A843P + | 30 | 90 | 168 | 172 |
| K875T + N892Y | | | | |
| Q109R + E229S + S635T + G753E + S754E + A769T + L775A + | 30 | 90 | 168 | 175 |
| K875T + N892Y | | | | |
| E229S + S578K + T631N + G753E + D801G | 30 | 90 | 168 | 176 |
| E229S + K360R + S578K + P752R + S754E + A769T + L775A + | 30 | 90 | 168 | 178 |
| K875T + N892Y | | | | |
| E229S + S578K + T923H | 30 | 90 | 88 | 178 |
| Q109R + E229S + G753E + S754E + A769T + L775A + K875T + | 30 | 90 | 168 | 180 |
| N892Y + K1016T | | | | |
| G753E + L775A + D801G + K875T + N892Y + V998K | 30 | 90 | 168 | 180 |
| E229S + S578K + T631N + G753E + K1016T | 30 | 90 | 168 | 180 |
| Q109R + E229S + S578K + G753E + S754E + A769T + L775A + | 30 | 90 | 168 | 182 |
| K875T + N892Y | | | | |
| E229S + S578K + L775A + K792Y | 30 | 90 | 88 | 184 |
| E229S + S578K + P752K + S754E | 30 | 90 | 88 | 184 |
| E229S + S578K + A911V + A912T + T923H | 30 | 90 | 88 | 186 |
| E229S + S578K + T631N + G753E + A769T + L775A | 30 | 90 | 168 | 187 |
| E229S + S578K + T631N + G753E + A769D + K792Y | 30 | 90 | 168 | 188 |
| E229S + S578K + P752K | 30 | 90 | 88 | 188 |
| P752K + G753E + S754E + L775A + D801G + K875T + N892Y | 30 | 90 | 168 | 190 |
| Q109R + E229S + G753E + S754E + A769T + L775A + K875T + | 30 | 90 | 168 | 191 |
| N892Y + A932P | | | | |
| E229S + A492L + S578K + G753E + L775A | 30 | 90 | 168 | 195 |
| P752K + G753E + S754E + L775A + D801G + K875T + N892Y | 30 | 90 | 167 | 195 |
| E229S + S578K + T631N + G753E + D901A | 30 | 90 | 168 | 197 |
| E229S + K360R + S578K + P752R + A769D + L775A + K875T + | 30 | 90 | 167 | 209 |
| N892Y | | | | |
| E229S + S578K + A911V | 30 | 90 | 88 | 210 |
| Q109R + E229S + A769T + L775A + D801G + K875T + N892Y | 30 | 90 | 168 | 221 |
| E229S + A492L + S578K + G753E + N1008D | 30 | 90 | 168 | 226 |
| E229S + A492L + S578K + G753E + L775A + P779V | 30 | 90 | 168 | 227 |
| E229S + A492L + S578K + G753E + A769D | 30 | 90 | 168 | 230 |
| E229S + S578K + T631N + G753E + A769D + A774V + L775A + | 30 | 90 | 168 | 234 |
| P779V + K792Y | | | | |
| A769D + L775A + D801G + K875T + N892Y + V998K | 30 | 90 | 168 | 238 |
| E229S + K360R + S578K + S754E + A769D + L775A + K875T + | 30 | 90 | 168 | 238 |
| N892Y | | | | |
| E229S + S578K + A769D + L775A + K875T + N892Y | 30 | 90 | 168 | 239 |
| S635T + A769D + L775A + D801G + K875T + N892Y | 30 | 90 | 168 | 243 |
| E229S + S578K + S754E | 30 | 90 | 88 | 243 |
| Q109R + E229S + G753E + S754E + A769E + L775A + K875T + | 30 | 90 | 168 | 249 |
| N892Y | | | | |
| E229S + A492L + S578K + G753E + L775A + K792Y | 30 | 90 | 168 | 259 |
| A769D + L775A + D801G + A843P + K875T + N892Y | 30 | 90 | 168 | 270 |
| G753E + S754E + L775A + D801G + K875T + N892Y | 30 | 90 | 168 | 276 |
| G753E + S754E + A769D + L775A + D801G + K875T + N892Y | 30 | 90 | 168 | 277 |
| E229S + K360R + S578K + P752K + A769D + L775A + K875T + | 30 | 90 | 168 | 280 |
| N892Y | | | | |
| E229S + S578K + T631N + G753E + A912T | 30 | 90 | 168 | 281 |
| Q109R + E229S + N672D + G753E + S754E + A769T + L775A + | 30 | 90 | 168 | 297 |
| K875T + N892Y | | | | |
| A190Q + A769D + L775A + D801G + K875T + N892Y | 30 | 90 | 168 | 297 |
| P752R + G753E + A769D + L775A + D801G + K875T + N892Y | 30 | 90 | 168 | 300 |
| P752R + G753E + S754E + A769D + L775A + D801G + K875T + | 30 | 90 | 168 | 341 |
| N892Y | | | | |
| A769D + L775A + D801G + K875T + N892Y + N1008D | 30 | 90 | 168 | 343 |
| G753E + A769D + L775A + D801G + K875T + N892Y | 30 | 90 | 168 | 344 |
| E229S + K360R + S578K + P752R + G753E + A769D + L775A + | 30 | 90 | 168 | 375 |
| K875T + N892Y | | | | |
| N672D + A769D + L775A + D801G + K875T + N892Y | 30 | 90 | 168 | 380 |
| N672D + G753E + L775A + D801G + K875T + N892Y | 30 | 90 | 168 | >385 |
| Q109R + G753E + S754E + A769T + L775A + D801G + K875T + | 30 | 90 | 168 | >385 |
| N892Y | | | | |
| E229S + G753E + L775A + D801G + K875T + N892Y | 30 | 90 | 168 | >385 |
| E229N + G753E + L775A + D801G + K875T + N892Y | 30 | 90 | 168 | >385 |
| P752K + G753E + S754E + A769D + L775A + D801G + K875T + | 30 | 90 | 168 | >385 |
| N892Y | | | | |
| E229N + A769D + L775A + D801G + K875T + N892Y | 30 | 90 | 168 | >385 |
| E229S + K360R + S578K + P752K + G753E + A769D + L775A + | 30 | 90 | 168 | >385 |
| K875T + N892Y | | | | |
| E229S + A769D + L775A + D801G + K875T + N892Y | 30 | 90 | 168 | >385 |
| E229S + K360R + S578K + G753E + S754E + A769D + L775A + | 30 | 90 | 168 | >385 |
| K875T + N892Y | | | | |
| Q109R + N672D + G753E + S754E + A769T + L775A + K875T + | 30 | 90 | 168 | >385 |
| N892Y | | | | |
| T631N + A769D + L775A + D801G + K875T + N892Y | 30 | 90 | 168 | >385 |
| Q109R + E229S + G753E + S754E + A769T + L775A + D801G + | 30 | 90 | 168 | >385 |
| K875T + N892Y | | | | |

TABLE 29
Half-life of purified variants: Temperature (T) 30° C., detergent concentration 95%

| | | | Incubation | |
|---|---|---|---|---|
| | T | Detergent | time | Half-life |
| Mutations relative to SEQ ID NO: 6 | (° C.) | (%) | (h) | (h) |
| No mutations (Wild-type) | 30 | 95 | 1 | <0.2 |
| E229N + N672D + P752K + G753E + A769D + L775A + D801G + | 30 | 95 | 672 | 281 |
| K875T + N892Y | | | | |
| Q109R + E229S + N672D + P752R + G753E + S754E + A769T + | 30 | 95 | 672 | 310 |
| L775A + D801G + K875T + N892Y | | | | |
| Q109R + E229S + N672D + P752R + G753E + S754E + A769T + | 30 | 95 | 672 | 316 |
| L775A + D801G + K875T + N892Y + D901A | | | | |
| Q109R + A159P + E229S + N672D + P752R + G753E + S754E + | 30 | 95 | 672 | 355 |
| A769T + L775A + D801G + K875T + N892Y | | | | |
| E229N + N672D + P752R + G753E + A769D + L775A + D801G + | 30 | 95 | 672 | 355 |
| K875T + N892Y | | | | |
| E229N + N672D + G753E + S754E + A769D + L775A + D801G + | 30 | 95 | 672 | 356 |
| K875T + N892Y + N1008D | | | | |
| Q109R + E229S + G753E + S754E + A769D + L775A + D801G + | 30 | 95 | 672 | 357 |
| K875T + N892Y + N1008D | | | | |
| Q109R + E229S + G753E + S754E + A769D + L775A + D801G + | 30 | 95 | 672 | 358 |
| K875T + N892Y | | | | |
| Q109R + E229S + N672D + G753E + S754E + A769T + L775A + | 30 | 95 | 672 | 359 |
| D801G + K875T + N892Y + T923H | | | | |
| Q109R + A159P + E229S + G753E + S754E + A769D + L775A + | 30 | 95 | 672 | 366 |
| D801G + A843P + K875T + N892Y | | | | |
| Q109R + A159P + E229S + S635E + T649K + G753E + S754E + | 30 | 95 | 672 | 373 |
| A769D + L775A + D801G + K875T + N892Y | | | | |
| Q109R + A190Q + E229S + N672D + G753E + S754E + A769T + | 30 | 95 | 672 | 383 |
| L775A + D801G + K875T + N892Y | | | | |
| Q109R + E229S + T631N + N672D + P752R + G753E + S754E + | 30 | 95 | 672 | 383 |
| A769T + L775A + D801G + K875T + N892Y | | | | |
| E229N + N672D + G753E + S754E + A769D + L775A + D801G + | 30 | 95 | 672 | 384 |
| K875T + N892Y | | | | |
| Q109R + E229S + N672D + I703L + G753E + S754E + A769T + | 30 | 95 | 672 | 399 |
| L775A + D801G + K875T + N892Y | | | | |
| E229N + P752K + G753E + A769D + L775A + D801G + A843P + | 30 | 95 | 672 | 404 |
| K875T + N892Y | | | | |
| Q109R + E229S + N672D + G753E + S754E + A769E + L775A + | 30 | 95 | 672 | 405 |
| D801G + K875T + N892Y | | | | |
| Q109R + A159P + E229S + N672D + G753E + S754E + A769T + | 30 | 95 | 672 | 421 |
| L775A + D801G + A843P + K875T + N892Y | | | | |
| Q109R + A159P + E229S + G753E + S754E + A769D + L775A + | 30 | 95 | 672 | 428 |
| D801G + K875T + N892Y | | | | |
| Q109R + A159P + E229S + I703L + G753E + S754E + A769D + | 30 | 95 | 672 | 433 |
| L775A + D801G + K875T + N892Y | | | | |
| Q109R + A159P + E229S + K567R + N672D + G753E + S754E + | 30 | 95 | 672 | 452 |
| A769T + L775A + D801G + K875T + N892Y | | | | |
| Q109R + A159P + E229S + N672D + G753E + S754E + A769T + | 30 | 95 | 672 | 454 |
| L775A + D801G + K875T + N892Y | | | | |
| Q109R + E229S + T631N + N672D + P752R + G753E + S754E + | 30 | 95 | 672 | 477 |
| A769D + L775A + D801G + A843P + K875T + N892Y | | | | |
| Q109R + A159P + E229S + T631N + G753E + S754E + A769D + | 30 | 95 | 672 | 481 |
| L775A + D801G + K875T + N892Y | | | | |
| Q109R + E229S + N672D + G753E + S754E + A769T + L775A + | 30 | 95 | 672 | 485 |
| D801G + A843P + K875T + N892Y | | | | |
| Q109R + E229S + N672D + I703L + P752R + G753E + S754E + | 30 | 95 | 672 | 499 |
| A769T + L775A + D801G + K875T + N892Y | | | | |
| Q109R + E229S + T631N + S635E + T649K + N672D + P752R + | 30 | 95 | 672 | 513 |
| G753E + S754E + A769D + L775A + D801G + K875T + N892Y | | | | |
| Q109R + A159P + E229S + A624E + A626G + S635E + T649K + | 30 | 95 | 672 | 519 |
| N672D + G753E + S754E + S757D + A769T + L775A + D801G + | | | | |
| K875T + N892Y | | | | |
| Q109R + A159P + E229S + S635E + T649K + N672D + I703L + | 30 | 95 | 672 | 529 |
| G753E + S754E + S757D + A769T + L775A + D801G + K875T + | | | | |
| N892Y | | | | |
| Q109R + E229S + T631N + N672D + P752R + G753E + S754E + | 30 | 95 | 672 | 562 |
| S757D + A769T + L775A + D801G + K875T + N892Y | | | | |
| Q109R + E229S + T631N + N672D + P752R + G753E + S754E + | 30 | 95 | 672 | 565 |
| S757D + A769D + L775A + D801G + K875T + N892Y | | | | |
| E229S + 1234V + S635E + T649K + I656V + N672D + G753E + | 30 | 95 | 672 | 611 |
| A769D + L775A + D801G + K875T + N892Y | | | | |
| Q109R + E229S + T631N + N672D + P752R + G753E + S754E + | 30 | 95 | 672 | 637 |
| S757D + A769D + L775A + D801G + K875T + N892Y + K1016T | | | | |
| E229S + S635E + T649K + I656V + N672D + S754E + A769D + | 30 | 95 | 672 | 662 |
| L775A + D801G + K875T + N892Y | | | | |
| E229S + A624E + S635E + T649K + 1656V + N672D + I703L + | 30 | 95 | 672 | 663 |
| G753E + S754E + A769D + L775A + D801G + K875T + N892Y + | | | | |
| N1008D | | | | |
| E229S + N440K + S582K + N672D + G753E + S754E + A769D + | 30 | 95 | 672 | 691 |
| L775A + D801G + K875T + N892Y + N1008D | | | | |
| T18D + Q109R + E229S + T631N + N672D + P752R + G753E + | 30 | 95 | 840 | 694 |
| S754E + S757D + A769D + L775A + D801G + K875T + N892Y | | | | |
| E229S + N440K + S582K + T631N + N672D + G753E + S754E + | 30 | 95 | 840 | 713 |
| A769D + L775A + D801G + K875T + N892Y + N1008D | | | | |
| L46D + E229S + K360G + D458S + S582K + N672D + G753E + | 30 | 95 | 840 | 714 |
| S754E + A769D + L775A + D801G + K875T + N892Y + N1008D | | | | |
| Q109R + E229S + K567R + T631N + N672D + P752R + G753E + | 30 | 95 | 840 | 732 |
| S754E + S757D + A769D + L775A + D801G + K875T + N892Y | | | | |
| S100D + E229S + S635E + T649K + 1656V + N672D + I703L + | 30 | 95 | 672 | 754 |
| G753E + A769D + L775A + D801G + A843P + K875T + N892Y + | | | | |
| N1008D | | | | |
| E229S + D458S + K567R + T631N + N672D + G753E + S754E + | 30 | 95 | 672 | 780 |
| A769D + L775A + D801G + K875T + N892Y | | | | |
| E229S + D458S + S582K + N672D + G753E + S754E + A769D + | 30 | 95 | 672 | 783 |
| L775A + D801G + K875T + N892Y + N1008D | | | | |
| E229S + S635E + T649K + I656V + N672D + G753E + S754E + | 30 | 95 | 672 | 796 |
| A769D + L775A + D801G + K875T + N892Y | | | | |
| E229S + N672D + G753E + S754E + A769D + L775A + D801G + | 30 | 95 | 672 | 827 |
| K875T + N 892Y | | | | |
| Q109R + E229S + K360G + T631N + N672D + P752R + G753E + | 30 | 95 | 840 | 831 |
| S754E + S757D + A769D + L775A + D801G + K875T + N892Y | | | | |
| E229N + T631N + N672D + G753E + S754E + A769D + L775A + | 30 | 95 | 672 | 834 |
| D801G + K875T + N892Y + N1008D | | | | |
| E229S + S635E + T649K + I656V + N672D + P752R + G753E + | 30 | 95 | 672 | 836 |
| S754E + A769D + L775A + D801G + K875T + N892Y | | | | |
| E229S + I234V + N672D + G753E + S754E + A769D + L775A + | 30 | 95 | 840 | 844 |
| D801G + K875T + N892Y | | | | |
| E229S + S635E + T649K + I656V + N672D + G753E + A769D + | 30 | 95 | 672 | 857 |
| L775A + D801G + K875T + N892Y | | | | |
| E229S + I234V + A492L + S582K + N672D + M728V + G753E + | 30 | 95 | 840 | 857 |
| S754E + A769D + L775A + D801G + K875T + N892Y | | | | |
| E229S + S635E + T649K + I656V + N672D + G753E + S754E + | 30 | 95 | 840 | 868 |
| A769D + L775A + D801G + K875T + N892Y | | | | |
| E229S + S635E + T649K + I656V + N672D + G753E + A769D + | 30 | 95 | 672 | 943 |
| L775A + D801G + K875T + N892Y | | | | |
| A159P + E229S + N440K + N672D + G753E + S754E + A769D + | 30 | 95 | 672 | 946 |
| L775A + D801G + K875T + N892Y | | | | |
| E229S + D458S + N672D + G753E + S754E + A769D + L775A + | 30 | 95 | 840 | 950 |
| D801G + K875T + N892Y | | | | |
| E229S + D458S + T631N + N672D + G753E + S754E + S757D + | 30 | 95 | 672 | 956 |
| A769D + L775A + D801G + K875T + N892Y | | | | |
| S100D + E229S + S635E + T649K + 1656V + N672D + P752K + | 30 | 95 | 672 | 956 |
| G753E + S754E + A769D + L775A + D801G + K875T + N892Y | | | | |
| E229S + D458S + T631E + N672D + G753E + S754E + A769D + | 30 | 95 | 840 | 961 |
| L775A + D801G + K875T + N892Y | | | | |
| E229S + S582K + N672D + G753E + S754E + A769D + L775A + | 30 | 95 | 672 | 966 |
| D801G + K875T + N892Y | | | | |
| E229S + S635E + T649K + I656V + N672D + P752K + G753E + | 30 | 95 | 672 | 978 |
| S754E + A769D + L775A + D801G + K875T + N892Y | | | | |
| E229S + D458S + S582K + T631E + N672D + G753E + S754E + | 30 | 95 | 840 | 993 |
| A769D + L775A + D801G + K875T + N892Y | | | | |
| A190Q + E229S + S635E + T649K + 1656V + N672D + I703L + | 30 | 95 | 672 | 1004 |
| G753E + S754E + S757D + A769D + L775A + D801G + K875T + | | | | |
| N892Y + N1008D | | | | |
| A190Q + E229S + S635E + T649K + 1656V + N672D + I703L + | 30 | 95 | 672 | 1015 |
| G753E + S754E + A769D + L775A + V800P + D801G + K875T + | | | | |
| N892Y + N1008D | | | | |
| A190Q + E229S + I234V + S582K + N672D + G753E + S754E + | 30 | 95 | 672 | 1019 |
| A769D + L775A + D801G + K875T + N892Y | | | | |
| T18D + E229S + S582K + N672D + G753E + S754E + A769D + | 30 | 95 | 840 | 1023 |
| L775A + D801G + K875T + N892Y | | | | |
| E229S + D458S + T631E + N672D + G753E + S754E + S757D + | 30 | 95 | 840 | 1030 |
| A769D + L775A + D801G + K875T + N892Y | | | | |
| Q89Y + E229S + N672D + G753E + S754E + A769D + L775A + | 30 | 95 | 840 | 1038 |
| D801G + K875T + N892Y | | | | |
| S100D + E229S + D458S + K567R + S635E + N672D + G753E + | 30 | 95 | 672 | 1041 |
| S754E + A769D + L775A + D801G + K875T + N892Y | | | | |
| E229S + S635E + T649K + 1656V + N672D + I703L + G753E + | 30 | 95 | 840 | 1045 |
| S754E + A769D + L775A + D801G + K875T + N892Y + N1008D | | | | |
| E229S + V3521 + S635E + T649K + 1656V + N672D + G753E + | 30 | 95 | 840 | 1065 |
| S754E + A769D + L775A + V800P + D801G + K875T + N892Y | | | | |
| E229S + N672D + P752R + G753E + S754E + A769D + L775A + | 30 | 95 | 672 | 1066 |
| D801G + K875T + N892Y | | | | |
| E229S + K360G + D458S + S582K + N672D + G753E + S754E + | 30 | 95 | 840 | 1067 |
| A769D + L775A + D801G + K875T + N892Y + N1008D | | | | |
| E229S + I234V + A492L + N672D + G753E + S754E + A769D + | 30 | 95 | 840 | 1070 |
| L775A + D777R + D801G + K875T + N892Y | | | | |
| E229S + S635E + T649K + N672D + G753E + S754E + A769D + | 30 | 95 | 840 | 1081 |
| L775A + D801G + K875T + N892Y + N1008D | | | | |
| E229S + N440K + S582K + A624E + N672D + P752R + G753E + | 30 | 95 | 624 | 1087 |
| S754E + A769D + L775A + D801G + K875T + N892Y + N1008D | | | | |
| E229S + I234V + S582K + N672D + G753E + S754E + A769D + | 30 | 95 | 792 | 1094 |
| L775A + V800P + D801G + K875T + N892Y | | | | |
| T18D + E229S + S582K + N672D + G753E + S754E + A769D + | 30 | 95 | 672 | 1101 |
| L775A + D801G + K875T + N892Y + T902F | | | | |
| Q89Y + E229S + N440K + S582K + A624E + N672D + G753E + | 30 | 95 | 672 | 1117 |
| S754E + A769D + L775A + D801G + K875T + N892Y | | | | |
| E229S + D458S + T631E + N672D + G753E + S754E + A769D + | 30 | 95 | 840 | 1121 |
| L775A + D777K + D801G + K875T + N892Y | | | | |
| E229S + S635E + T649K + I656V + N672D + P752K + G753E + | 30 | 95 | 672 | 1141 |
| A769D + L775A + D801G + A843P + K875T + N892Y | | | | |
| E229S + S635E + T649K + I656V + N672D + G753E + S754E + | 30 | 95 | 672 | 1145 |
| S757D + A769D + L775A + D801G + K875T + N892Y | | | | |
| E229S + N440K + S582K + N672D + G753E + S754E + A769D + | 30 | 95 | 672 | 1146 |
| L775A + D801G + A843P + K875T + N892Y + N1008D | | | | |
| S100D + E229S + S635E + T649K + 1656V + N672D + G753E + | 30 | 95 | 672 | 1153 |
| S754E + A769D + L775A + D801G + K875T + N892Y | | | | |
| L46D + E229S + K360R + S578K + N672D + G753E + S754E + | 30 | 95 | 840 | 1172 |
| A769D + L775A + D801G + K875T + N892Y | | | | |
| E229S + T631N + N672D + I703L + P752K + G753E + A769D + | 30 | 95 | 840 | 1183 |
| L775A + D801G + A843P + K875T + N892Y | | | | |
| E229S + A624E + S635E + T649K + I656V + N672D + G738L + | 30 | 95 | 792 | 1192 |
| G753E + 5754R + S757D + A769D + L775A + D777K + D801G + | | | | |
| K875T + N892Y | | | | |
| E229S + N440K + S582K + A624E + N672D + G753E + S754E + | 30 | 95 | 624 | 1214 |
| A769D + L775A + V800P + D801G + K875T + N892Y | | | | |
| E229S + D458S + K567R + S635E + N672D + G753E + S754E + | 30 | 95 | 624 | 1226 |
| A769D + L775A + D801G + K875T + N892Y | | | | |
| A190Q + E229S + S635E + T649K + 1656V + N672D + I703L + | 30 | 95 | 672 | 1238 |
| P752R + G753E + S754E + A769D + L775A + D801G + K875T + | | | | |
| N892Y + N1008D | | | | |
| E229S + T631N + N672D + I703L + P752K + G753E + S754E + | 30 | 95 | 840 | 1259 |
| A769D + L775A + D801G + K875T + N892Y | | | | |
| A190Q + E229S + T631N + N672D + I703L + P752K + G753E + | 30 | 95 | 840 | 1269 |
| A769D + L775A + D801G + K875T + N892Y | | | | |
| A190Q + E229S + S635E + T649K + I656V + N672D + P752K + | 30 | 95 | 672 | 1282 |
| G753E + A769D + L775A + D801G + A843P + K875T + N892Y | | | | |
| A159P + A190Q + E229S + I234V + S582K + N672D + G753E + | 30 | 95 | 672 | 1289 |
| S754E + A769D + L775A + D801G + K875T + N892Y | | | | |
| E229S + A492L + S635E + T649K + I656V + N672D + P752R + | 30 | 95 | 672 | 1298 |
| G753E + S754E + A769D + L775A + D801G + K875T + N892Y + | | | | |
| N1008D | | | | |
| A190Q + E229S + S582K + N672D + G753E + S754E + A769D + | 30 | 95 | 792 | 1299 |
| L775A + D801G + K875T + N892Y | | | | |
| T18D + E229S + S582K + N672D + G753E + S754E + P764K + | 30 | 95 | 624 | 1299 |
| 6A79D + L775A + D801G + K875T + N892Y | | | | |
| E229S + N440K + S582K + N672D + P752R + G753E + S754E + | 30 | 95 | 624 | 1328 |
| 7S57D + A769D + L775A + D801G + K875T + N892Y + N1008D | | | | |
| E229S + A492L + S635E + T649K + I656V + N672D + G753E + | 30 | 95 | 840 | 1329 |
| A769D + L775A + D801G + K875T + N892Y | | | | |
| E229S + S582K + S635E + N672D + P752R + G753E + S754E + | 30 | 95 | 792 | 1352 |
| A769D + L775A + D801G + K875T + N892Y + N1008D | | | | |
| A190Q + E229S + N440K + S582K + A624E + S635E + N672D + | 30 | 95 | 672 | 1358 |
| G753E + S754E + A769D + L775A + D801G + K875T + N892Y | | | | |
| A190Q + E229S + D458S + T631N + N672D + G753E + S754E + | 30 | 95 | 624 | 1398 |
| A769D + L775A + D801G + K875T + N892Y | | | | |
| E229S + I234V + A492L + S582K + N672D + G753E + S754E + | 30 | 95 | 840 | 1426 |
| A769D + L775A + D801G + K875T + N892Y | | | | |
| E229S + N440K + S582K + S635E + N672D + G753E + S754E + | 30 | 95 | 840 | 1456 |
| A769D + L775A + D801G + A843P + K875T + N892Y + N1008D | | | | |
| S100D + A190Q + E229S + 1234V + S582K + N672D + G753E + | 30 | 95 | 672 | 1460 |
| S754E + A769D + L775A + D801G + K875T + N892Y | | | | |
| A190Q + E229S + K360G + D458S + S582K + T664K + N672D + | 30 | 95 | 792 | 1481 |
| G753E +S754E + A769D + L775A + D801G + K875T + N892Y + | | | | |
| N1008D | | | | |
| S100D + E229S + N440K + S582K + N672D + P752R + G753E + | 30 | 95 | 840 | 1489 |
| S754E + A769D + L775A + D801G + K875T + N892Y + N1008D | | | | |
| E229S + S635E + T649K + I656V + N672D + P752K + G753E + | 30 | 95 | 672 | 1519 |
| S757D + A769D + L775A + D801G + A843P + K875T + N892Y | | | | |
| S100D + E229S + K360G + D458S + S582K + N672D + G753E + | 30 | 95 | 792 | 1530 |
| S754E + S757D + A769D + L775A + D801G + A843P + K875T + | | | | |
| N892Y + T915A + N1008D | | | | |
| E229S + N440K + S582K + A624E + S635E + N672D + G738L + | 30 | 95 | 792 | 1535 |
| G753E + S754E + S757D + A769D + L775A + D801G + K875T + | | | | |
| N892Y | | | | |
| S100D + E229S + K360G + D458S + S582K + N672D + G753E + | 30 | 95 | 840 | 1538 |
| S754E + A769D + L775A + D801G + K875T + N892Y + N1008D | | | | |
| A190Q + E229S + D458S + T631N + N672D + G753E + S754E + | 30 | 95 | 672 | 1551 |
| A769D + L775A + D801G + A843P + K875T + N892Y | | | | |
| A190Q + E229S + K360G + D458S + S582K + N672D + G753E + | 30 | 95 | 840 | 1556 |
| S754E + A769D + L775A + D801G + K875T + N892Y + N1008D | | | | |
| E229S + S582K + S635E + T649K + I656V + N672D + M728V + | 30 | 95 | 672 | 1594 |
| G753E + 5754R + S757D + A769D + L775A + D801G + K875T + | | | | |
| N892Y | | | | |
| A190Q + E229S + N440K + S582K + A624E + S635E + N672D + | 30 | 95 | 792 | 1601 |
| G753E + S754E + S757D + A769D + L775A + D801G + K875T + | | | | |
| N892Y | | | | |
| S100D + A190Q + E229S + S635E + T649K + 1656V + N672D + | 30 | 95 | 624 | 1606 |
| I703L + G753E + S754E + A769D + L775A + D801G + K875T + | | | | |
| N892Y + N1008D | | | | |
| S100D + E229S + D458S + K567R + S582K + S635E + N672D + | 30 | 95 | 792 | 1612 |
| G753E + S754E + A769D + L775A + D801G + K875T + N892Y | | | | |
| E229S + S582K + S635E + T649K + I656V + N672D + P752K + | 30 | 95 | 624 | 1619 |
| G753E + S757D + A769D + L775A + D801G + A843P + K875T + | | | | |
| N892Y | | | | |
| E229S + S635E + T649K + I656V + N672D + G753E + S754R + | 30 | 95 | 672 | 1664 |
| S757D + A769D + L775A + D801G + A843P + K875T + N892Y | | | | |
| T18D + E229S + D458S + T631N + N672D + M728V + G753E + | 30 | 95 | 792 | 1700 |
| S754E + S757D + A769D + L775A + D801G + K875T + N892Y | | | | |
| E229S + D458S + S582K + T631N + S635E + N672D + M728V + | 30 | 95 | 792 | 1704 |
| G753E + S754E + S757D + A769D + L775A + D801G + K875T + | | | | |
| N892Y | | | | |
| A190Q + E229S + K360G + D458S + S582K + N672D + G753E + | 30 | 95 | 792 | 1714 |
| S754E + S757D + A769D + L775A + D801G + K875T + N892Y + | | | | |
| N1008D | | | | |
| S100D + E229S + K360G + D458S + S582K + N672D + G753E + | 30 | 95 | 792 | 1741 |
| S754E + S757D + A769D + L775A + D801G + A843P + K875T + | | | | |
| N892Y + N1008D | | | | |
| E229S + S635E + N672D + P752R + G753E + S754E + A769D + | 30 | 95 | 792 | 1745 |
| L775A + D777K + D801G + K875T + N892Y | | | | |
| A159P + E229S + D458S + T631N + N672D + M728V + G753E + | 30 | 95 | 792 | 1783 |
| S754E + S757D + A769D + L775A + D801G + K875T + N892Y | | | | |
| E229S + A492L + S635E + T649K + I656V + N672D + G753E + | 30 | 95 | 792 | 1826 |
| S757D + A769D + L775A + D801G + K875T + N892Y | | | | |
| S100D + A190Q + E229S + K360G + D458S + S582K + N672D + | 30 | 95 | 792 | 1844 |
| G753E + S754E + A769D + L775A + D801G + K875T + N892Y + | | | | |
| N1008D | | | | |
| S100D + E229S + N440K + S582K + T631N + N672D + G753E + | 30 | 95 | 624 | 1907 |
| S754E + A769D + L775A + D801G + K875T + N892Y + N1008D | | | | |
| E229S + I234V + A492L + N672D + G753E + S754E + A769D + | 30 | 95 | 624 | 1924 |
| L775A + D777K + D801G + K875T + N892Y | | | | |
| A190Q + E229S + I234V + S582K + N672D + G753E + S754E + | 30 | 95 | 672 | 1993 |
| S757D + A769D + L775A + D801G + K875T + N892Y | | | | |
| E229S + D458S + A492L + T631N + N672D + G753E + S754E + | 30 | 95 | 624 | 2378 |
| S757D + A769D + L775A + D801G + K875T + N892Y | | | | |
| E229S + A624E + S635E + T649K + I656V + N672D + G753E + | 30 | 95 | 624 | 2380 |
| S754R + S757D + A769D + L775A + D777K + D801G + K875T + | | | | |
| N892Y | | | | |
| S100D + E229S + K360G + D458S + S582K + N672D + G753E + | 30 | 95 | 792 | 3039 |
| S754E + S757D + A769D + L775A + D801G + A843P + K875T + | | | | |
| N892Y + N1008D | | | | |
| E229S + D458S + K567R + S582K + S635E + T649K + N672D + | 30 | 95 | 840 | 674 |
| G753E + S754E + A769D + L775A + D777R + D801G + K875T + | | | | |
| N892Y | | | | |
| S100D + E229S + K360G + D458S + S582K + T664K + N672D + | 30 | 95 | 840 | 1023 |
| G753E + 5754R + S757D + A769D + L775A + D801G + A843P + | | | | |
| K875T + N892Y + N1008D + K1016T | | | | |
| S100D + E229S + K360G + D458S + S582K + T664K + N672D + | 30 | 95 | 840 | 963 |
| G753E + S754E + S757D + A769D + L775A + D801G + A843P + | | | | |
| K875T + N892Y + N1008D + K1016T | | | | |
| S100D + E229S + K360G + D458S + S582K + N672D + G753E + | 30 | 95 | 840 | 982 |
| S754E + S757D + A769D + L775A + D801G + A843P + K875T + | | | | |
| N892Y + A911V + N1008D | | | | |
| S100D + E229S + K360G + D458S + S582K + N672D + G753E + | 30 | 95 | 840 | 906 |
| S754E + S757D + A769D + L775A + D801G + A843P + K875T + | | | | |
| N892Y + A911V + N1008D + K1016T | | | | |
| S100D + E229S + K360G + D458S + S582K + S635E + N672D + | 30 | 95 | 840 | 918 |
| G753E + S754E + S757D + A769D + L775A + D801G + A843P + | | | | |
| K875T + N892Y + N1008D | | | | |
| S100D + E229S + K360G + D458S + S582K + T664K + N672D + | 30 | 95 | 840 | 962 |
| G753E + S754E + S757D + A769D + L775A + D801G + A843P + | | | | |
| K875T + N892Y + A911V + N1008D + K1016T | | | | |
| S100D + E229S + K360G + D458S + S582K + N672D + G753E + | 30 | 95 | 840 | 888 |
| S754E + S757D + A769D + L775A + D801G + A843P + K875T + | | | | |
| N892Y + N1008D + K1016T | | | | |
| S100D + E229S + K360G + D458S + S582K + T664K + N672D + | 30 | 95 | 840 | 1091 |
| G753E + S754E + S757D + A769D + L775A + D801G + A843P + | | | | |
| K875T + N892Y + A911V + N1008D + K1016T | | | | |
| S100D + E229S + K360G + D458S + S582K + N672D + G753E + | 30 | 95 | 840 | 796 |
| S754E + S757D + A769D + L775A + D801G + A843P + K875T + | | | | |
| N892Y + N1008D + K1016T | | | | |
| S100D + E229S + K360G + D458S + S582K + S635E + T649K + | 30 | 95 | 840 | 984 |
| N672D + G753E + S754E + S757D + A769D + L775A + D801G + | | | | |
| A843P + K875T + N892Y + N1008D | | | | |
| S100D + E229S + K360G + D458S + S582K + N672D + G753E + | 30 | 95 | 840 | 867 |
| S754E + S757D + A769D + L775A + D801G + A843P + K875T + | | | | |
| N892Y + N1008D | | | | |
| S100D + E229S + K360G + D458S + S582K + N672D + G753E + | 30 | 95 | 840 | 876 |
| S754E + S757D + A769D + L775A + D801G + A843P + K875T + | | | | |
| N892Y | | | | |
| E229S + N440K + S582K + A624E + S635E + N672D + G738L + | 30 | 95 | 840 | 689 |
| G753E + S754E + S757D + A769D + L775A + D801G + K875T + | | | | |
| N892Y | | | | |
| E229S + N399K + D458S + A492H + K567R + S582K + S635E + | 30 | 95 | 840 | 2256 |
| T649K + N672D + G753E + S754E + A769D + L775A + D777R + | | | | |
| D801G + K875T + N892Y | | | | |
| A190Q + E229S + I234V + T505I + S582K + N672D + G753E + | 30 | 95 | 840 | 698 |
| S754E + S757D + A769D + L775A + D801G + K875T + N892Y | | | | |
| E229S + D458S + A492H + K567R + S582K + S635E + T649K + | 30 | 95 | 840 | 492 |
| N672D + G753E + S754E + A769D + L775A + D777R + D801G + | | | | |
| K875T + N892Y | | | | |
| E229S + N399K + D458S + K567R + S582K + S635E + N672D + | 30 | 95 | 840 | 3540 |
| G753E + S754E + A769D + L775A + D777R + D801G + K875T + | | | | |
| N892Y | | | | |
| E229S + D458S + A492H + K567R + S582K + S635E + N672D + | 30 | 95 | 840 | 2837 |
| G753E + S754E + A769D + L775A + D777R + D801G + K875T + | | | | |
| N892Y | | | | |
| E229S + N399K + D458S + A492H + K567R + S582K + S635E + | 30 | 95 | 840 | 536 |
| N672D + G753E + S754E + A769D + L775A + D777R + D801G + | | | | |
| K875T + N892Y | | | | |
| E229S + N399K + D458S + K567R + S582K + S635E + T649K + | 30 | 95 | 840 | 2830 |
| N672D + G753E + S754E + A769D + L775A + D777R + D801G + | | | | |
| K875T + N892Y | | | | |
| A190Q + E229S + I234V + S582K + N672D + G753E + S754E + | 30 | 95 | 840 | 874 |
| S757D + A769D + L775A + D801G + K875T + N892Y | | | | |
| E229S + N440K + S582K + A624E + S635E + N672D + G738L + | 30 | 95 | 840 | 768 |
| G753E + S754E + S757D + A769D + L775A + D801G + K875T + | | | | |
| N892Y | | | | |
| S100D + E229S + K360G + D458S + S582K + N672D + G753E + | 30 | 95 | 840 | 883 |
| S754E + S757D + A769D + L775A + D801G + A843P + K875T + | | | | |
| N892Y + N1008D | | | | |
| E229S + N399K + D458S + A492H + K567R + S582K + S635E + | 30 | 95 | 840 | 1836 |
| T649K + N672D + G753E + S754E + A769D + L775A + D777R + | | | | |
| D801G + K875T + N892Y | | | | |

TABLE 30
Half-life of purified variants: Temperature (T) 32° C., detergent concentration 90%

| | | | Incubation | Half- |
|---|---|---|---|---|
| | T | Detergent | time | life |
| Mutations relative to SEQ ID NO: 6 | (° C.) | (%) | (h) | (h) |
| No mutations (Wild-type) | 32 | 90 | 1 | <0.2 |
| Q109R + A769T + L775A + K792Y + K875T | 32 | 90 | 20 | 12 |
| L775A + K875T + N892Y + A911V + D933M | 32 | 90 | 20 | 13 |
| Q109R + L775A + K792Y + K875T | 32 | 90 | 20 | 13 |
| L775A + K875T + N892Y | 32 | 90 | 20 | 14 |
| L775A + S851F + K875T + N892Y + A911V | 32 | 90 | 20 | 14 |
| L775A + K875T + N892Y + A911V + A912T + T923H | 32 | 90 | 20 | 14 |
| L775A + K875T + N892Y + D933M | 32 | 90 | 20 | 15 |
| E229S + S578R + A769D + L775A + K819R | 32 | 90 | 20 | 16 |
| E229S + K291R + S578R + P752R + G753E | 32 | 90 | 20 | 17 |
| E229S + L775A + K875T + N892Y | 32 | 90 | 20 | 17 |
| E229S + K291R + S578R + G753E | 32 | 90 | 20 | 17 |
| Q109R + S578K + D801G + K875T | 32 | 90 | 20 | 17 |
| E229S + S578R + L775A + K819R | 32 | 90 | 20 | 18 |
| E229S + S578R + D801G + K819R | 32 | 90 | 20 | 18 |
| E229S + S578R + K792Y + K819R | 32 | 90 | 20 | 18 |
| Q109R + L775A + K792Y + K875T + N892Y | 32 | 90 | 20 | 19 |
| E229S + S578R + A769D + K792Y + K819R | 32 | 90 | 20 | 19 |
| S578K + L775A + K875T + N892Y | 32 | 90 | 20 | 20 |
| E229S + S578R + P752R + G753E + N892Y | 32 | 90 | 20 | 20 |
| E229S + S578R + S754E + N892Y | 32 | 90 | 20 | 20 |
| E229S + S578R + L775A + N892Y | 32 | 90 | 20 | 21 |
| E229S + S578R + L775A + K792Y + K819R | 32 | 90 | 20 | 21 |
| E229S + S578R + G753E + N892Y | 32 | 90 | 20 | 21 |
| Q109R + L775A + K875T + N892Y | 32 | 90 | 20 | 21 |
| E229S + K291R + S578R + P752K + G753E | 32 | 90 | 20 | 21 |
| E229S + K291R + S578R + P752R | 32 | 90 | 20 | 21 |
| E229S + S578K + K819R | 32 | 90 | 20 | 22 |
| E229S + S578R + P779V + K819R | 32 | 90 | 20 | 22 |
| E229S + S578R + K819R | 32 | 90 | 20 | 23 |
| Q109R + A769T + L775A + K792Y + D801G + K819R + N892Y | 32 | 90 | 20 | 23 |
| Q109R + A769T + L775A + D801G + K819R + N892Y | 32 | 90 | 20 | 25 |
| E229S + S578R + P752K + G753E + N892Y | 32 | 90 | 20 | 25 |
| E229S + S578R + A769Q + K819R | 32 | 90 | 20 | 25 |
| E229S + S578R + P752K + S754E + N892Y | 32 | 90 | 20 | 27 |
| Q109R + L775A + K875T + N892Y | 32 | 90 | 20 | 28 |
| Q109R + A769T + L775A + K792Y + K875T + N892Y | 32 | 90 | 20 | 31 |
| Q109R + A769T + L775A + K875T + N892Y | 32 | 90 | 20 | 33 |
| L775A + D801G + K875T + N892Y | 32 | 90 | 20 | 36 |
| Q109R + L775A + P779V + K792Y + D801G + K819R + K875T + N892Y | 32 | 90 | 20 | 37 |
| Q109R + A769T + L775A + K875T + N892Y | 32 | 90 | 20 | 38 |
| Q109R + E229S + A769T + L775A + K875T + N892Y | 32 | 90 | 20 | 45 |
| Q109R + P752R + G753E + A769T + L775A + K875T + N892Y | 32 | 90 | 20 | 55 |
| Q109R + P752K + G753E + S754E + A769T + L775A + K875T + N892Y | 32 | 90 | 20 | 56 |
| Q109R + S754E + A769T + L775A + K875T + N892Y | 32 | 90 | 20 | 58 |
| Q109R + P752R + G753E + S754E + A769T + L775A + K875T + N892Y | 32 | 90 | 20 | 58 |
| Q109R + P752K + G753E + A769T + L775A + K875T + N892Y | 32 | 90 | 20 | 60 |
| Q109R + G753E + A769T + L775A + K875T + N892Y | 32 | 90 | 20 | 61 |
| Q109R + G753E + S754E + A769T + L775A + K875T + N892Y | 32 | 90 | 20 | 62 |
| Q109R + N672D + G753E + S754E + A769T + L775A + K875T + N892Y | 32 | 90 | 167 | 95 |
| E229S + K360R + S578K + P752R + A769D + L775A + K875T + | 32 | 90 | 167 | 98 |
| N892Y + A911V | | | | |
| E229S + K360R + S578R + N672D + P752R + A769D + L775A + | 32 | 90 | 167 | 98 |
| K875T + N892Y | | | | |
| N672D + G753E + L775A + D801G + K875T + N892Y + A911V | 32 | 90 | 167 | 108 |
| E229S + K360R + S578K + N672D + P752R + A769D + L775A + | 32 | 90 | 167 | 116 |
| K875T + N892Y | | | | |
| N672D + A769D + L775A + D801G + K875T + N892Y | 32 | 90 | 167 | 120 |
| T631N + N672D + A769D + L775A + D801G + K875T + N892Y | 32 | 90 | 167 | 122 |
| N672D + G753E + L775A + D801G + K875T + N892Y + N1008D | 32 | 90 | 167 | 123 |
| A190Q + N672D + A769D + L775A + D801G + K875T + N892Y | 32 | 90 | 167 | 124 |
| Q109R + G753E + S754E + A769T + L775A + D801G + K875T + N892Y | 32 | 90 | 167 | 126 |
| Q89Y + N672D + A769D + L775A + D801G + K875T + N892Y | 32 | 90 | 167 | 129 |
| N672D + G753E + L775A + D801G + A843P + K875T + N892Y | 32 | 90 | 167 | 131 |
| L46D + E229S + K360R + S578K + N672D + P752R + A769D + | 32 | 90 | 167 | 131 |
| L775A + K875T + N892Y | | | | |
| E229S + K360R + S578K + N672D + P752R + A769D + L775A + | 32 | 90 | 167 | 133 |
| K875T + N892Y | | | | |
| E229S + K360R + S578K + N672D + P752R + A769D + L775A + | 32 | 90 | 167 | 135 |
| K875T + N892Y + A912T | | | | |
| E229S + K360R + S578K + P752K + G753E + S754E + A769D + | 32 | 90 | 167 | 136 |
| L775A + K875T + N892Y | | | | |
| E229S + S578K + A769D + L775A + K875T + N892Y | 32 | 90 | 167 | 138 |
| E229S + S578K + P752K + S754E + K875T + N892Y | 32 | 90 | 167 | 140 |
| E229S + K567R + S578K + A769D + L775A + K875T + N892Y | 32 | 90 | 167 | 141 |
| E229S + K360R + S578K + P752R + A769D + L775A + D801G + | 32 | 90 | 167 | 155 |
| K875T + N892Y | | | | |
| N672D + G753E + L775A + D801G + K875T + N892Y | 32 | 90 | 167 | 160 |
| E229S + S578K + A769D + L775A + P779V + K792Y + K875T + N892Y | 32 | 90 | 167 | 167 |
| E229S + K360R + S578K + T631N + N672D + P752R + A769D + | 32 | 90 | 167 | 168 |
| L775A + K875T + N892Y | | | | |
| N672D + A769D + L775A + D801G + A843P + K875T + N892Y | 32 | 90 | 167 | 171 |
| E229S + K360R + S578K + N672D + P752R + A769D + L775A + | 32 | 90 | 167 | 175 |
| K875T + N892Y + A932P | | | | |
| E229S + A492L + S578K + T631N + G753E | 32 | 90 | 167 | 176 |
| E229S + S578K + P752K + G753E + A769D + L775A + K875T + | 32 | 90 | 167 | 182 |
| N892Y | | | | |
| E229S + K360R + S578K + N672D + P752R + G753E + A769D + | 32 | 90 | 167 | 185 |
| L775A + K875T + N892Y | | | | |
| N672D + G753E + S754E + A769D + L775A + D801G + K875T + N892Y | 32 | 90 | 167 | 187 |
| E229S + S578K + P752K + G753E + S754E + A769D + L775A + | 32 | 90 | 167 | 196 |
| K875T + N892Y | | | | |
| E229S + K360R + S578K + N672D + P752R + G753E + S754E + | 32 | 90 | 167 | 205 |
| A769D + L775A + K875T + N892Y | | | | |
| E229S + K360R + S578K + N672D + P752K + G753E + S754E + | 32 | 90 | 167 | 216 |
| A769D + L775A + K875T + N892Y | | | | |
| E229S + S578K + G753E + A769D + L775A + K875T + N892Y | 32 | 90 | 167 | 218 |
| E229S + K360R + S578K + N672D + G753E + S754E + A769D + | 32 | 90 | 167 | 221 |
| L775A + D845E + K875T + N892Y | | | | |
| Q109R + E229S + S578K + P752K | 32 | 90 | 167 | 225 |
| E229S + S578K + G753E + S754E + A769D + L775A + K875T + N892Y | 32 | 90 | 167 | 234 |
| E229S + K360R + S578K + N672D + P752K + G753E + A769D + | 32 | 90 | 167 | 234 |
| L775A + K875T + N892Y | | | | |
| E229S + S578K + N672D + A769D + L775A + K875T + N892Y | 32 | 90 | 167 | 239 |
| E229S + S578K + P752R + G753E + S754E + A769D + L775A + | 32 | 90 | 167 | 278 |
| K875T + N892Y | | | | |
| Q109R + E229S + G753E + S754E + A769T + L775A + D801G + | 32 | 90 | 167 | 308 |
| K875T + N892Y | | | | |
| E229S + S578K + A769D + L775A + D801G + K875T + N892Y | 32 | 90 | 167 | 337 |
| E229S + N672D + G753E + L775A + D801G + K875T + N892Y | 32 | 90 | 167 | 367 |
| E229N + N672D + A769D + L775A + D801G + K875T + N892Y | 32 | 90 | 167 | >385 |
| E229S + N672D + A769D + L775A + D801G + K875T + N892Y | 32 | 90 | 167 | >385 |

TABLE 31
Half-life of purified variants: Temperature (T) 35° C., detergent concentration 90%

| | | | Incubation | Half- |
|---|---|---|---|---|
| | T | Detergent | time | life |
| Mutations relative to SEQ ID NO: 6 | (° C.) | (%) | (h) | (h) |
| No mutations (Wild-type) | 35 | 90 | 1 | <0.2 |
| E229N + P752R + G753E + S754E + L775A + D801G + K875T + N892Y | 35 | 90 | 71 | 27 |
| E229N + G753E + L775A + D801G + K875T + N892Y | 35 | 90 | 71 | 27 |
| Q109R + E229S + K451R + A769T + L775A + D801G + K875T + N892Y | 35 | 90 | 70 | 28 |
| E229S + S578K + G753E + A912T | 35 | 90 | 70 | 28 |
| T631N + P752R + G753E + S754E + A769D + L775A + D801G + | 35 | 90 | 71 | 30 |
| E845D + K875T + N892Y | | | | |
| E229S + K360R + S578K + P752R + S754E + A769D + L775A + | 35 | 90 | 71 | 30 |
| K875T + N892Y | | | | |
| Q89Y + Q109R + E229S + A769T + I775A + D801G + K875T + N892Y | 35 | 90 | 70 | 30 |
| N672D + A769D + L775A + D801G + K875T + N892Y | 35 | 90 | 70 | 33 |
| E229N + A769D + L775A + D801G + K875T + N892Y | 35 | 90 | 71 | 33 |
| Q109R + N672D + G753E + S754E + A769T + L775A + K875T + N892Y | 35 | 90 | 70 | 33 |
| Q109R + E229S + S578K + P779V | 35 | 90 | 70 | 34 |
| N672D + G753E + L775A + D801G + K875T + N892Y + A911V | 35 | 90 | 70 | 34 |
| E229S + S578K + G753E + A912T | 35 | 90 | 70 | 34 |
| S100D + Q109R + N672D + G753E + S754E + A769T + L775A + | 35 | 90 | 70 | 35 |
| K875T + N892Y | | | | |
| E2295 + K360R + S578K + S635E + T649K + P752R + A769D + | 35 | 90 | 71 | 38 |
| L775A + D801G + K875T + N892Y | | | | |
| Q109R + G753E + S754E + A769E + L775A + D801G + K875T + N892Y | 35 | 90 | 71 | 38 |
| E2295 + K360R + S578K + N672D + P752R + S754E + A769D + | 35 | 90 | 71 | 40 |
| L775A + K875T + N892Y | | | | |
| Q109R + E2295 + S578K + A912T | 35 | 90 | 70 | 40 |
| E2295 + K360R + S578K + P752R + A769D + L775A + D801G + | 35 | 90 | 71 | 42 |
| K875T + N892Y | | | | |
| Q109R + A159P + E2295 + N672D + F746L + G753E + S754E + | 35 | 90 | 168 | 42 |
| A769T + L775A + D801G + A843P + K875T + N892Y | | | | |
| E2295 + S578K + G753E + P779V + K792Y + D801G + A912T | 35 | 90 | 70 | 42 |
| E2295 + K360R + S578K + P752R + S754E + A769D + L775A + | 35 | 90 | 71 | 44 |
| D801G + K875T + N892Y | | | | |
| E2295 + T631N + A769D + L775A + D801G + K875T + N892Y | 35 | 90 | 166 | 44 |
| E2295 + S578K + P752K + G753E + S754E + D801G + A912T | 35 | 90 | 70 | 46 |
| E229N + S754E + A769D + L775A + D801G + K875T + N892Y | 35 | 90 | 71 | 47 |
| E229N + N672D + P752K + G753E + A769D + L775A + D801G + | 35 | 90 | 119 | 48 |
| K875T + N892Y + N991D | | | | |
| E229N + P752K + G753E + A769D + L775A + D801G + K875T + N892Y | 35 | 90 | 120 | 48 |
| S100D + E2295 + K360R + S578K + T631N + P752R + G753E + | 35 | 90 | 119 | 50 |
| S754E + A769D + L775A + D801G + K875T + N892Y | | | | |
| E2295 + K360R + S578K + T631N + P752R + G753E + S754E + | 35 | 90 | 119 | 51 |
| A769D + L775A + D801G + K875T + N892Y + N991D | | | | |
| E229N + T631N + N672D + A769D + L775A + D801G + K875T + N892Y | 35 | 90 | 70 | 52 |
| A58L + E2295 + K360R + S578K + N672D + G753E + S754E + | 35 | 90 | 70 | 52 |
| A769D + L775A + K875T + N892Y | | | | |
| E2295 + K360R + S578K + N672D + G753E + S754E + A769D + | 35 | 90 | 70 | 56 |
| L775A + D845E + K875T + N892Y | | | | |
| Q109R + N672D + G753E + S754E + A769E + L775A + D801G + | 35 | 90 | 71 | 56 |
| K875T + N892Y | | | | |
| E2295 + K360R + S578K + T631N + N672D + P752R + G753E + | 35 | 90 | 119 | 57 |
| S754E + A769D + L775A + D801G + K875T + N892Y | | | | |
| E2295 + S578K + N672D + A769D + L775A + K875T + N892Y | 35 | 90 | 120 | 58 |
| L46D + E2295 + K360R + S578K + N672D + G753E + S754E + | 35 | 90 | 70 | 58 |
| A769D + L775A + A843P + K875T + N892Y | | | | |
| E229N + S635E + T649K + I656V + N672D + A769D + L775A + | 35 | 90 | 166 | 59 |
| D801G + K875T + N892Y | | | | |
| L46D + E2295 + K360R + S578K + T631N + N672D + G753E + | 35 | 90 | 70 | 59 |
| S754E + A769D + L775A + K875T + N892Y | | | | |
| E229S + S578K + P752K + G753E + S754E + A912T | 35 | 90 | 166 | 61 |
| Q109R + E2295 + G753E + S754E + A769T + L775A + D801G + | 35 | 90 | 166 | 61 |
| K875T + N892Y + A911V | | | | |
| E2295 + K360R + S578K + P752R + G753E + S754E + A769D + | 35 | 90 | 166 | 62 |
| L775A + D801G + K875T + N892Y | | | | |
| Q109R + E229S + S578K + P752R + G753E | 35 | 90 | 166 | 62 |
| Q109R + E229S + A769T + L775A + D801G + K875T + N892Y | 35 | 90 | 166 | 62 |
| L46D + E229S + S578K + G753E + S754E + A769D + L775A + | 35 | 90 | 167 | 63 |
| D801G + K875T + N892Y | | | | |
| Q109R + E229S + T631N + G753E + S754E + A769T + L775A + | 35 | 90 | 166 | 63 |
| D801G + K875T + N892Y | | | | |
| Q109R + E229S + D458S + G753E + S754E + A769T + L775A + | 35 | 90 | 166 | 64 |
| D801G + K875T + N892Y | | | | |
| Q109R + E229S + G753E + S754E + A769T + L775A + D801G + | 35 | 90 | 166 | 64 |
| K875T + N892Y | | | | |
| S100D + E229S + S578K + G753E + A912T | 35 | 90 | 166 | 64 |
| E229S + K360R + S578K + P752K + G753E + S754E + A769D + | 35 | 90 | 71 | 64 |
| L775A + D801G + K875T + N892Y | | | | |
| E229N + P752K + G753E + S754E + A769D + L775A + D801G + | 35 | 90 | 166 | 64 |
| K875T + N892Y | | | | |
| E229N + S635E + T649K + I656V + N672D + G753E + A769D + | 35 | 90 | 168 | 64 |
| L775A + D801G + K875T + N892Y + N991D | | | | |
| Q109R + E229S + S635T + G753E + S754E + A769T + L775A + | 35 | 90 | 166 | 65 |
| D801G + K875T + N892Y | | | | |
| E229N + S635E + T649K + I656V + N672D + S757D + A769D + | 35 | 90 | 168 | 65 |
| L775A + D801G + A843P + K875T + N892Y | | | | |
| E229N + N672D + P752R + S754E + A769D + L775A + D801G + | 35 | 90 | 70 | 66 |
| K875T + N892Y | | | | |
| E229N + K567R + S635E + T649K + I656V + N672D + G753E + | 35 | 90 | 168 | 66 |
| A769D + L775A + D801G + K875T + N892Y | | | | |
| E229S + S578K + G753E + D801G + A912T | 35 | 90 | 166 | 66 |
| Q109R + E229S + G753E + S754E + A769T + L775A + D801G + | 35 | 90 | 166 | 66 |
| K875T + N892Y + D901A | | | | |
| Q109R + A190Q + E229S + G753E + S754E + A769T + L775A + | 35 | 90 | 166 | 66 |
| D801G + K875T + N892Y | | | | |
| Q109R + E229S + A769T + L775A + D801G + A843P + K875T + N892Y | 35 | 90 | 166 | 66 |
| E229N + I234V + S635E + T649K + I656V + N672D + A769D + | 35 | 90 | 120 | 66 |
| L775A + D801G + A843P + K875T + N892Y | | | | |
| E229N + S635E + T649K + I656V + N672D + A769D + L775A + | 35 | 90 | 168 | 66 |
| D801G + A843P + K875T + N892Y | | | | |
| Q109R + E229S + G753E + S754E + A769T + L775A + D801G + | 35 | 90 | 166 | 67 |
| K875T + N892Y + N1008D | | | | |
| T631N + A769D + L775A + D801G + K875T + N892Y | 35 | 90 | 166 | 67 |
| Q109R + E229S + A492L + A769T + L775A + D801G + K875T + N892Y | 35 | 90 | 166 | 67 |
| E229N + P752K + G753E + A769D + L775A + D801G + A843P + | 35 | 90 | 168 | 68 |
| K875T + N892Y | | | | |
| E229S + S578N + N672D + A769D + L775A + D801G + K875T + N892Y | 35 | 90 | 168 | 68 |
| 727\+ E229S + K360R + S578K + N672D + G753E + S754E + | 35 | 90 | 166 | 69 |
| A769D + L775A + D801G + K875T + N892Y | | | | |
| E229S + P752K + G753E + A769D + L775A + D801G + K875T + N892Y | 35 | 90 | 168 | 69 |
| E229N + S635E + T649K + I656V + N672D + S754E + A769D + | 35 | 90 | 166 | 69 |
| L775A + D801G + K875T + N892Y | | | | |
| L46D + E229S + I234V + K360R + S578K + N672D + G753E + | 35 | 90 | 168 | 69 |
| S754E + A769D + L775A + D801G + K875T + N892Y | | | | |
| E229N + I234V + N672D + G753E + S754E + A769D + L775A + | 35 | 90 | 168 | 70 |
| D801G + K875T + N892Y + N1008D | | | | |
| L46D + E229S + K360R + S578K + N672D + G753E + S754E + | 35 | 90 | 168 | 70 |
| A769D + L775A + D801G + K875T + N892Y | | | | |
| E229N + N672D + P752K + A769D + L775A + D801G + K875T + N892Y | 35 | 90 | 166 | 70 |
| Q109R + E229S + P752K + G753E + S754E + A769T + L775A + | 35 | 90 | 166 | 70 |
| D801G + K875T + N892Y | | | | |
| E229N + N672D + A769D + L775A + D801G + K875T + N892Y | 35 | 90 | 70 | 70 |
| Q109R + E229S + T631N + S635E + T649K + N672D + P752R + | 35 | 90 | 167 | 71 |
| G753E + S754E + A769T + L775A + D801G + K875T + N892Y | | | | |
| Q109R + E229S + G753E + S754E + A769D + L775A + P779V + | 35 | 90 | 166 | 71 |
| D801G + K875T + N892Y | | | | |
| E229N + D458S + N672D + G753E + S754E + A769D + L775A + | 35 | 90 | 166 | 72 |
| D801G + K875T + N892Y | | | | |
| L46D + E229S + K360R + S578K + N672D + G753E + S754E + | 35 | 90 | 168 | 72 |
| A769D + L775A + D801G + K875T + N892Y | | | | |
| A58L + E229N + N672D + G753E + S754E + A769D + L775A + | 35 | 90 | 166 | 73 |
| D801G + K875T + N892Y | | | | |
| A159P + E229N + N672D + G753E + S754E + A769D + L775A + | 35 | 90 | 166 | 73 |
| D801G + K875T + N892Y | | | | |
| E229N + S635E + T649K + I656V + N672D + G753E + S754E + | 35 | 90 | 166 | 74 |
| A769D + L775A + D801G + K875T + N892Y | | | | |
| E229N + N672D + G753E + S754E + A769D + L775A + D801G + | 35 | 90 | 168 | 75 |
| K875T + N892Y + N1008D | | | | |
| E229S + K360R + S578K + N672D + P752R + A769D + L775A + | 35 | 90 | 71 | 75 |
| D801G + K875T + N892Y | | | | |
| Q109R + A159P + E229S + S635E + G753E + S754E + A769D + | 35 | 90 | 167 | 75 |
| L775A + D801G + K875T + N892Y | | | | |
| E229S + S578K + N672D + G753E + S754E + A769D + L775A + | 35 | 90 | 168 | 75 |
| K875T + N892Y | | | | |
| E229N + S635E + T649K + I656V + N672D + G753E + A769D + | 35 | 90 | 168 | 76 |
| L775A + D801G + K875T + N892Y + T923H | | | | |
| E229S + T631N + P752K + G753E + A769D + L775A + D801G + | 35 | 90 | 167 | 76 |
| K875T + N892Y | | | | |
| E229N + S635E + T649K + I656V + N672D + P752R + G753E + | 35 | 90 | 166 | 77 |
| S754E + A769D + L775A + D801G + K875T + N892Y | | | | |
| E229N + S635E + T649K + I656V + N672D + P752K + G753E + | 35 | 90 | 168 | 77 |
| A769D + L775A + D801G + A843P + K875T + N892Y | | | | |
| A190Q + E229S + K360R + S578K + P752R + G753E + S754E + | 35 | 90 | 168 | 77 |
| A769D + L775A + D801G + K875T + N892Y | | | | |
| A58L + E229S + I234V + N672D + G753E + S754E + A769D + | 35 | 90 | 168 | 78 |
| L775A + D801G + K875T + N892Y | | | | |
| L46D + E229S + K360R + S578K + N672D + G753E + S754E + | 35 | 90 | 70 | 78 |
| A769D + L775A + K875T + N892Y + A912T | | | | |
| L46D + E229S + K360R + S578K + N672D + P752K + G753E + | 35 | 90 | 70 | 78 |
| S754E + A769D + L775A + K875T + N892Y | | | | |
| E229S + N672D + G753E + L775A + D801G + K875T + N892Y + | 35 | 90 | 166 | 78 |
| A911V | | | | |
| A159P + E229N + N672D + G753E + S754E + A769D + L775A + | 35 | 90 | 166 | 79 |
| D801G + K875T + N892Y + N1008D | | | | |
| E229N + S635E + N672D + G753E + S754E + A769D + L775A + | 35 | 90 | 168 | 80 |
| D801G + K875T + N892Y + N1008D | | | | |
| E229N + S635E + T649K + I656V + N672D + A769D + L775A + | 35 | 90 | 120 | 80 |
| D801G + A843P + K875T + N892Y + N991D | | | | |
| E229N + T631N + N672D + G753E + S754E + A769D + L775A + | 35 | 90 | 166 | 80 |
| D801G + K875T + N892Y | | | | |
| E229N + N672D + P752K + G753E + S754E + A769D + L775A + | 35 | 90 | 168 | 80 |
| D801G + K875T + N892Y + N1008D | | | | |
| E229N + N672D + G753E + S754E + A769D + L775A + D801G + | 35 | 90 | 166 | 80 |
| K875T + N892Y + V998K | | | | |
| Q109R + E229S + K567R + A769T + L775A + D801G + K875T + N892Y | 35 | 90 | 166 | 81 |
| E229N + S635T + N672D + G753E + S754E + A769D + L775A + | 35 | 90 | 166 | 82 |
| D801G + K875T + N892Y | | | | |
| Q109R + A159P + E229S + S635E + T649K + G753E + S754E + | 35 | 90 | 167 | 82 |
| A769D + L775A + D801G + K875T + N892Y + N1008D | | | | |
| E229N + N672D + G753E + S754E + A769D + L775A + D801G + | 35 | 90 | 166 | 82 |
| A843P + K875T + N892Y | | | | |
| E229N + N672D + I703L + A769D + L775A + D801G + K875T + | 35 | 90 | 166 | 82 |
| N892Y | | | | |
| E229S + K360R + S578K + T631N + P752R + G753E + S754E + | 35 | 90 | 120 | 82 |
| A769D + L775A + D801G + K875T + N892Y | | | | |
| E229N + N672D + I703L + G753E + S754E + A769D + L775A + | 35 | 90 | 166 | 82 |
| D801G + K875T + N892Y | | | | |
| E229S + S635E + T649K + I656V + N672D + A769D + L775A + | 35 | 90 | 168 | 83 |
| D801G + K875T + N892Y | | | | |
| Q109R + E229S + N672D + P752R + G753E + S754E + A769S + | 35 | 90 | 168 | 83 |
| L775A + D801G + K875T + N892Y | | | | |
| Q109R + A159P + E229S + L5331 + S582K + N672D + M728V + | 35 | 90 | 168 | 84 |
| G753E + S754E + A769T + L775A + D801G + A843P + K875T + | | | | |
| N892Y | | | | |
| Q109R + A159P + E229S + N672D + G753E + S754E + A769T + | 35 | 90 | 168 | 84 |
| L775A + D801G + K875T + N892Y + A911V | | | | |
| E229N + S635E + T649K + I656V + N672D + P752K + G753E + | 35 | 90 | 166 | 84 |
| A769D + L775A + D801G + K875T + N892Y | | | | |
| Q109R + A159P + E229S + G753E + S754E + A769T + L775A + | 35 | 90 | 166 | 85 |
| D801G + K875T + N892Y | | | | |
| E229N + S635E + T649K + I656V + N672D + G753E + A769D + | 35 | 90 | 168 | 86 |
| L775A + D801G + K875T + N892Y | | | | |
| E229S + K567R + P752K + G753E + A769D + L775A + D801G + | 35 | 90 | 167 | 86 |
| K875T + N892Y | | | | |
| E229N + N672D + G753E + A769D + L775A + D801G + K875T + N892Y | 35 | 90 | 166 | 87 |
| Q109R + E229S + T631N + G753E + S754E + A769D + L775A + | 35 | 90 | 166 | 87 |
| D801G + K875T + N892Y | | | | |
| L46D + E229S + K360R + S578K + A624E + N672D + G753E + | 35 | 90 | 168 | 87 |
| S754E + A769D + L775A + D801G + K875T + N892Y | | | | |
| Q109R + A159P + E229S + S635E + T649K + G753E + S754E + | 35 | 90 | 167 | 87 |
| A769D + L775A + D801G + K875T + N892Y + A912T | | | | |
| E229N + T631N + S635E + T649K + I656V + N672D + A769D + | 35 | 90 | 70 | 88 |
| L775A + D801G + K875T + N892Y | | | | |
| Q109R + E229S + K451R + N672D + G753E + S754E + A769T + | 35 | 90 | 166 | 88 |
| L775A + D801G + K875T + N892Y | | | | |
| E229N + S635E + T649K + I656V + N672D + P752K + G753E + | 35 | 90 | 166 | 89 |
| S754E + A769D + L775A + D801G + K875T + N892Y | | | | |
| S100D + Q109R + A159P + E229S + S635E + T649K + G753E + | 35 | 90 | 167 | 89 |
| S754E + A769D + L775A + D801G + K875T + N892Y | | | | |
| E229N + S635E + T649K + I656V + N672D + P752R + G753E + | 35 | 90 | 168 | 89 |
| A769D + L775A + D801G + A843P + K875T + N892Y | | | | |
| Q109R + E2295 + N672D + P752R + G753E + S754E + A769T + | 35 | 90 | 168 | 90 |
| L775A + D801G + K875T + N892Y + A911V | | | | |
| E229N + K567R + S635E + T649K + I656V + N672D + A769D + | 35 | 90 | 70 | 90 |
| L775A + D801G + K875T + N892Y | | | | |
| Q109R + A159P + E229S + N672D + G753E + S754E + A769T + | 35 | 90 | 168 | 90 |
| L775A + D801G + K875T + N892Y | | | | |
| E229N + N672D + P752R + G753E + S754E + A769D + L775A + | 35 | 90 | 166 | 90 |
| D801G + K875T + N892Y | | | | |
| E229S + S635E + T649K + I656V + N672D + G753E + S754E + | 35 | 90 | 149 | 90 |
| A769D + L775A + V800P + D801G + K875T + N892Y | | | | |
| Q109R + E229S + S635E + T649K + G753E + S754E + A769D + | 35 | 90 | 166 | 91 |
| L775A + D801G + K875T + N892Y | | | | |
| Q109R + A159P + E229S + G753E + S754E + A769D + L775A + | 35 | 90 | 168 | 91 |
| D801G + K875T + N892Y | | | | |
| Q109R + E229S + N672D + P752R + G753E + S754E + A769T + | 35 | 90 | 168 | 91 |
| L775A + D801G + K875T + N892Y + A912T | | | | |
| Q109R + A190Q + E229S + G753E + S754E + A769D + L775A + | 35 | 90 | 166 | 91 |
| D801G + K875T + N892Y | | | | |
| Q109R + A159P + A190Q + E229S + G753E + S754E + A769D + | 35 | 90 | 168 | 92 |
| L775A + D801G + K875T + N892Y | | | | |
| E229S + P752K + G753E + A769D + L775A + D801G + A843P + | 35 | 90 | 168 | 92 |
| K875T + N892Y | | | | |
| Q109R + A159P + E229S + S635E + T649K + P752R + G753E + | 35 | 90 | 167 | 92 |
| S754E + A769D + L775A + D801G + K875T + N892Y | | | | |
| E229N + N672D + G753E + S754E + A769D + L775A + D801G + | 35 | 90 | 168 | 93 |
| K875T + N892Y + N1008D | | | | |
| Q109R + E229S + S635T + G753E + S754E + A769D + L775A + | 35 | 90 | 166 | 95 |
| D801G + K875T + N892Y | | | | |
| Q109R + A159P + E229S + D458S + G753E + S754E + A769D + | 35 | 90 | 168 | 95 |
| L775A + D801G + K875T + N892Y | | | | |
| E229N + N672D + P752K + G753E + A769D + L775A + D801G + | 35 | 90 | 168 | 96 |
| A843P + K875T + N892Y | | | | |
| E229N + N672D + P752R + G753E + A769D + L775A + D801G + | 35 | 90 | 166 | 96 |
| K875T + N892Y | | | | |
| E229N + N672D + G753E + S754E + A769D + L775A + D801G + | 35 | 90 | 166 | 97 |
| K875T + N892Y | | | | |
| E229S + S582K + S635E + T649K + I656V + N672D + G738L + | 35 | 90 | 168 | 97 |
| G753E + S754E + A769D + L775A + D801G + K875T + N892Y | | | | |
| Q109R + A159P + E229S + S635E + T649K + N672D + P752K + | 35 | 90 | 167 | 97 |
| G753E + S754E + A769T + L775A + D801G + K875T + N892Y | | | | |
| Q109R + E229S + T631N + N672D + P752K + G753E + S754E + | 35 | 90 | 167 | 98 |
| A769T + L775A + D801G + K875T + N892Y | | | | |
| E229N + T631N + N672D + P752K + G753E + A769D + L775A + | 35 | 90 | 167 | 98 |
| D801G + K875T + N892Y | | | | |
| E229N + S635E + T649K + I656V + N672D + P752R + G753E + | 35 | 90 | 167 | 100 |
| S754E + A769D + L775A + D801G + A843P + K875T + N892Y | | | | |
| E229N + T631N + N672D + G753E + S754E + A769D + L775A + | 35 | 90 | 168 | 100 |
| D801G + K875T + N892Y + N1008D | | | | |
| E229N + N672D + P752K + G753E + A769D + L775A + D801G + | 35 | 90 | 167 | 101 |
| K875T + N892Y + T923H | | | | |
| Q89Y + Q109R + A159P + E229S + S635E + T649K + N672D + | 35 | 90 | 167 | 101 |
| G753E + S754E + A769T + L775A + D801G + K875T + N892Y | | | | |
| A190Q + E229S + S635E + T649K + 1656V + N672D + I703L + | 35 | 90 | 168 | 102 |
| G753E + S754E + A769D + L775A + D801G + K875T + N892Y + | | | | |
| N1008D | | | | |
| Q109R + E229S + N672D + P752R + G753E + S754E + A769T + | 35 | 90 | 168 | 103 |
| L775A + D801G + K875T + N892Y | | | | |
| Q109R + E229S + N672D + G753E + S754E + A769T + L775A + | 35 | 90 | 166 | 103 |
| D801G + K875T + N892Y | | | | |
| Q109R + E229S + G753E + S754E + A769D + L775A + D801G + | 35 | 90 | 166 | 103 |
| K875T + N892Y | | | | |
| Q109R + A159P + E229S + K567R + G753E + S754E + A769D + | 35 | 90 | 168 | 103 |
| L775A + D801G + K875T + N892Y | | | | |
| E229S + D458S + K567R + N672D + G753E + S754E + A769D + | 35 | 90 | 168 | 103 |
| L775A + D801G + K875T + N892Y | | | | |
| E229S + K360R + S578K + P752R + G753E + S754E + A769D + | 35 | 90 | 120 | 104 |
| L775A + D801G + K875T + N892Y | | | | |
| A159P + E229N + S635E + T649K + I656V + N672D + A769D + | 35 | 90 | 70 | 104 |
| L775A + D801G + K875T + N892Y | | | | |
| L46D + Q109R + A159P + E229S + N672D + G753E + S754E + | 35 | 90 | 168 | 104 |
| A769T + L775A + D801G + A843P + K875T + N892Y | | | | |
| Q109R + A159P + E229S + D458S + N672D + G753E + S754E + | 35 | 90 | 168 | 104 |
| A769T + L775A + D801G + K875T + N892Y | | | | |
| Q109R + A159P + E229S + S635E + T649K + G753E + S754E + | 35 | 90 | 167 | 104 |
| A769D + L775A + D801G + K875T + N892Y + K1016T | | | | |
| Q109R + E229S + N672D + P752R + G753E + S754E + A769T + | 35 | 90 | 168 | 107 |
| L775A + D801G + K875T + N892Y | | | | |
| L46D + A58L + E229S + K360R + S578K + N672D + G753E + | 35 | 90 | 70 | 108 |
| S754 E + A769D + L775A + K875T + N892Y | | | | |
| E229S + S635E + T649K + I656V + N672D + G753E + A769D + | 35 | 90 | 168 | 109 |
| L775A + D801G + K875T + N892Y | | | | |
| Q109R + E229S + N672D + G753E + S754E + A769T + L775A + | 35 | 90 | 166 | 109 |
| D801G + K875T + N892Y + N991D | | | | |
| Q109R + A159P + E229S + S635E + T649K + N672D + G753E + | 35 | 90 | 168 | 109 |
| S754E + A769T + L775A + D801G + K875T + N892Y | | | | |
| Q109R + A159P + E229S + S635E + T649K + G753E + S754E + | 35 | 90 | 168 | 110 |
| A769D + L775A + D801G + K875T + N892Y | | | | |
| Q109R + E229S + N672D + G753E + S754E + A769T + L775A + | 35 | 90 | 166 | 110 |
| D801G + K875T + N892Y + T923H | | | | |
| L46D + E229S + P752K + G753E + S757D + A769D + L775A + | 35 | 90 | 149 | 110 |
| D801G + K875T + N892Y | | | | |
| A190Q + E229N + N672D + G753E + S754E + A769D + L775A + | 35 | 90 | 166 | 112 |
| D801G + K875T + N892Y | | | | |
| E229S + S635E + T649K + I656V + N672D + S754E + A769D + | 35 | 90 | 168 | 112 |
| L775A + D801G + K875T + N892Y | | | | |
| Q109R + E229S + G753E + S754E + A769D + L775A + D801G + | 35 | 90 | 166 | 112 |
| K875T + N892Y + N1008D | | | | |
| E229N + N672D + I703L + P752K + G753E + A769D + L775A + | 35 | 90 | 167 | 113 |
| D801G + K875T + N892Y | | | | |
| E229S + T631N + S635E + T649K + 1656V + N672D + I703L + | 35 | 90 | 168 | 113 |
| G753E + S754E + A769D + L775A + D801G + K875T + N892Y + | | | | |
| N1008D | | | | |
| E229S + S635E + T649K + 1656V + N672D + I703L + M728V + | 35 | 90 | 168 | 114 |
| G753E + S754E + A769D + L775A + D801G + K875T + N892Y + | | | | |
| N1008D | | | | |
| Q109R + A159P + E229S + G753E + S754E + A769D + L775A + | 35 | 90 | 168 | 114 |
| D801G + K875T + N892Y + A912T | | | | |
| E229N + N672D + P752K + G753E + A769D + L775A + D801G + | 35 | 90 | 168 | 114 |
| K875T + N892Y | | | | |
| E229S + S635E + T649K + 1656V + N672D + I703L + G753E + | 35 | 90 | 168 | 114 |
| S754E + A769D + L775A + D801G + K875T + N892Y + N1008D | | | | |
| E229S + K360G + N672D + G753E + S754E + A769D + L775A + | 35 | 90 | 168 | 114 |
| D801G + K875T + N892Y | | | | |
| Q109R + A159P + E229S + K567R + N672D + G753E + S754E + | 35 | 90 | 168 | 114 |
| A769T + L775A + D801G + K875T + N892Y | | | | |
| Q109R + E229S + N672D + G753E + S754E + A769T + L775A + | 35 | 90 | 166 | 115 |
| D801G + K875T + N892Y + K1016T | | | | |
| L46D + E229S + N672D + G753E + S754E + A769D + L775A + | 35 | 90 | 168 | 115 |
| D801G + K875T + N892Y | | | | |
| Q109R + A159P + E229S + G753E + S754E + A769D + L775A + | 35 | 90 | 168 | 115 |
| D801G + K875T + N892Y + N1008D | | | | |
| Q109R + A159P + E229S + N672D + G753E + S754E + A769T + | 35 | 90 | 168 | 116 |
| L775A + V800P + D801G + A843P + K875T + N892Y | | | | |
| Q109R + A159P + E229S + G753E + S754E + A769D + L775A + | 35 | 90 | 168 | 116 |
| D801G + A843P + K875T + N892Y | | | | |
| Q109R + E229S + G753E + S754E + A769D + L775A + D801G + | 35 | 90 | 166 | 117 |
| K875T + N892Y | | | | |
| Q109R + A159P + E229S + I703L + G753E + S754E + A769D + | 35 | 90 | 168 | 117 |
| L775A + D801G + K875T + N892Y | | | | |
| Q109R + E229S + S582K + N672D + G753E + S754E + A769T + | 35 | 90 | 149 | 117 |
| L775A + D801G + A843P + K875T + N892Y | | | | |
| Q109R + E229S + S635E + T649K + 1656V + N672D + I703L + | 35 | 90 | 168 | 117 |
| G753E + S754E + A769D + L775A + D801G + K875T + N892Y + | | | | |
| N1008D | | | | |
| Q109R + A159P + E229S + S635E + T649K + N672D + G753E + | 35 | 90 | 167 | 117 |
| S754E + A769T + L775A + D801G + K875T + N892Y | | | | |
| E229S + S635E + T649K + I656V + N672D + P752K + G753E + | 35 | 90 | 168 | 117 |
| A769D + L775A + D801G + K875T + N892Y + A912T | | | | |
| Q109R + E229S + K567R + T631N + N672D + P752R + G753E + | 35 | 90 | 149 | 117 |
| S754E + A769D + L775A + D801G + K875T + N892Y | | | | |
| Q109R + A190Q + E229S + N672D + G753E + S754E + A769T + | 35 | 90 | 166 | 118 |
| L775A + D801G + K875T + N892Y | | | | |
| Q109R + A159P + E229S + T631N + G753E + S754E + A769D + | 35 | 90 | 168 | 118 |
| L775A + D801G + K875T + N892Y | | | | |
| E229S + N440K + S582K + A624E + N672D + G753E + S754E + | 35 | 90 | 168 | 120 |
| A769D + L775A + D801G + K875T + N892Y | | | | |
| Q109R + E229S + N672D + I703L + P752R + G753E + S754E + | 35 | 90 | 168 | 120 |
| A769T + L775A + D801G + K875T + N892Y | | | | |
| A190Q + E229S + D458S + N672D + G753E + S754E + A769D + | 35 | 90 | 168 | 120 |
| L775A + D801G + K875T + N892Y | | | | |
| E229S + S635E + T649K + I656V + N672D + G753E + A769D + | 35 | 90 | 168 | 121 |
| L775A + D801G + K875T + N892Y | | | | |
| Q109R + E229S + A624E + N672D + G753E + S754E + A769T + | 35 | 90 | 149 | 121 |
| L775A + D801G + A843P + K875T + N892Y | | | | |
| E229S + S635E + T649K + I656V + N672D + P752R + G753E + | 35 | 90 | 168 | 121 |
| S754E + A769D + L775A + D801G + K875T + N892Y | | | | |
| E229S + S635E + T649K + I656V + N672D + G753E + A769D + | 35 | 90 | 168 | 122 |
| L775A + D801G + K875T + N892Y + K1016T | | | | |
| Q109R + E229S + N672D + G753E + S754E + A769T + L775A + | 35 | 90 | 166 | 122 |
| D801G + K875T + N892Y + V998K | | | | |
| E229N + S635E + T649K + I656V + N672D + P752R + S754E + | 35 | 90 | 70 | 122 |
| A769D + L775A + D801G + K875T + N892Y | | | | |
| E229S + D458S + T631N + N672D + G753E + S754E + A769D + | 35 | 90 | 168 | 122 |
| L775A + D801G + K875T + N892Y | | | | |
| E229S + D458S + S635E + N672D + G753E + S754E + A769D + | 35 | 90 | 168 | 126 |
| L775A + D801G + K875T + N892Y + N1008D | | | | |
| Q109R + E229S + T631N + N672D + P752R + G753E + S754E + | 35 | 90 | 168 | 126 |
| A769T + L775A + D801G + K875T + N892Y | | | | |
| E229S + K360G + S635E + T649K + I656V + N672D + G753E + | 35 | 90 | 168 | 126 |
| A769D + L775A + D801G + K875T + N892Y | | | | |
| E229N + T631N + N672D + G753E + S754E + A769D + L775A + | 35 | 90 | 167 | 126 |
| D801G + K875T + N892Y + N1008D | | | | |
| Q109R + A159P + E229S + S635E + T649K + G753E + S754E + | 35 | 90 | 167 | 127 |
| A769D + L775A + D801G + A843P + K875T + N892Y | | | | |
| A159P + E229S + N672D + G753E + S754E + A769D + L775A + | 35 | 90 | 168 | 127 |
| D801G + K875T + N892Y | | | | |
| E229S + S635E + T649K + I656V + N672D + S757D + A769D + | 35 | 90 | 168 | 128 |
| L775A + D801G + K875T + N892Y | | | | |
| E229N + S635E + T649K + I656V + N672D + P752R + A769D + | 35 | 90 | 70 | 128 |
| L775A + D801G + K875T + N892Y | | | | |
| A159P + E229S + I234V + N672D + G753E + S754E + A769D + | 35 | 90 | 168 | 128 |
| L775A + D801G + K875T + N892Y | | | | |
| Q109R + E229S + N672D + P752R + G753E + S754E + A769T + | 35 | 90 | 168 | 129 |
| L775A + D801G + K875T + N892Y + D901A | | | | |
| E229S + S635E + T649K + I656V + N672D + G753E + A769D + | 35 | 90 | 168 | 130 |
| L775A + V800P + D801G + K875T + N892Y | | | | |
| L46D + E229S + K360G + S578K + N672D + G753E + S754E + | 35 | 90 | 168 | 130 |
| A769D + L775A + D801G + K875T + N892Y | | | | |
| Q109R + A159P + E229S + T631E + S635E + T649K + P752R + | 35 | 90 | 149 | 133 |
| G753E + S754E + A769D + L775A + D801G + K875T + N892Y | | | | |
| E229S + N672D + G753E + S754E + A769D + L775A + D801G + | 35 | 90 | 168 | 135 |
| K875T + N892Y | | | | |
| E229S + I234V + N672D + G753E + S754E + A769D + L775A + | 35 | 90 | 168 | 135 |
| D801G + K875T + N892Y | | | | |
| L46D + E229S + S635E + T649K + I656V + N672D + G753E + | 35 | 90 | 149 | 136 |
| S754E + A769D + L775A + D801G + K875T + N892Y | | | | |
| Q109R + E229S + T631N + N672D + P752R + G753E + S754E + | 35 | 90 | 167 | 136 |
| A769D + L775A + D801G + K875T + N892Y | | | | |
| S100D + A190Q + E229S + I234V + N672D + G753E + S754E + | 35 | 90 | 168 | 137 |
| A769D + L775A + D801G + K875T + N892Y | | | | |
| E229S + S635E + T649K + I656V + N672D + G753E + S754E + | 35 | 90 | 168 | 138 |
| A769D + L775A + D801G + K875T + N892Y | | | | |
| E229N + N672D + P752R + G753E + A769D + L775A + D801G + | 35 | 90 | 166 | 138 |
| K875T + N892Y | | | | |
| E229S + P752K + G753E + S757D + A769D + L775A + D801G + | 35 | 90 | 167 | 139 |
| K875T + N892Y | | | | |
| E229S + A624E + N672D + G753E + S754E + A769D + L775A + | 35 | 90 | 168 | 139 |
| D801G + K875T + N892Y | | | | |
| E229S + S635E + T649K + 1656V + N672D + I703L + G753E + | 35 | 90 | 168 | 139 |
| S754E + A769D + L775A + D801G + K875T + N892Y + N1008D | | | | |
| Q109R + E229S + D458S + N672D + G753E + S754E + A769T + | 35 | 90 | 166 | 140 |
| L775A + D801G + K875T + N892Y | | | | |
| A190Q + E229S + I234V + N672D + G753E + S754E + A769D + | 35 | 90 | 168 | 140 |
| L775A + D801G + K875T + N892Y | | | | |
| E229S + S635E + T649K + I656V + N672D + P752R + G753E + | 35 | 90 | 167 | 141 |
| S754E + A769D + L775A + D801G + K875T + N892Y | | | | |
| Q109R + A159P + E229S + S635E + T649K + N672D + G753E + | 35 | 90 | 167 | 141 |
| S754E + S757D + A769T + L775A + D801G + K875T + N892Y | | | | |
| Q109R + E229S + N672D + I703L + G753E + S754E + A769T + | 35 | 90 | 166 | 141 |
| L775A + D801G + K875T + N892Y | | | | |
| Q109R + E229S + T631N + N672D + G753E + S754E + A769T + | 35 | 90 | 166 | 141 |
| L775A + D801G + K875T + N892Y | | | | |
| E229S + T631E + P752K + G753E + S757D + A769D + L775A + | 35 | 90 | 168 | 141 |
| D801G + K875T + N892Y | | | | |
| E229S + N440K + S582K + N672D + G753E + S754E + S757D + | 35 | 90 | 168 | 141 |
| A769D + L775A + D801G + K875T + N892Y + N1008D | | | | |
| E229S + K567R + S635E + T649K + I656V + N672D + G753E + | 35 | 90 | 168 | 142 |
| A769D + L775A + D801G + K875T + N892Y | | | | |
| E229S + S635E + T649K + I656V + N672D + P752K + G753E + | 35 | 90 | 168 | 142 |
| A769D + L775A + D801G + K875T + N892Y + T923H + N1008D | | | | |
| Q109R + A159P + E229S + N672D + G753E + S754E + A769T + | 35 | 90 | 168 | 142 |
| L775A + D801G + K875T + N892Y | | | | |
| E229S + A624E + S635E + T649K + 1656V + N672D + I703L + | 35 | 90 | 168 | 143 |
| G753E + S754E + A769D + L775A + D801G + K875T + N892Y + | | | | |
| N1008D | | | | |
| A159P + E229S + S635E + T649K + I656V + N672D + G753E + | 35 | 90 | 168 | 145 |
| A769D + L775A + D801G + K875T + N892Y | | | | |
| E229S + S635E + T649K + I656V + N672D + P752K + G753E + | 35 | 90 | 168 | 145 |
| S754E + A769D + L775A + D801G + K875T + N892Y | | | | |
| Q109R + E229S + A624E + T631N + N672D + P752R + G753E + | 35 | 90 | 149 | 146 |
| S754E + A769D + L775A + D801G + K875T + N892Y | | | | |
| Q109R + A159P + E229S + S635E + T649K + N672D + G753E + | 35 | 90 | 168 | 146 |
| S754E + S757D + A769T + L775A + D801G + K875T + N892Y + | | | | |
| N1008D | | | | |
| E229S + S582K + S635E + T649K + I656V + N672D + G753E + | 35 | 90 | 168 | 146 |
| A769D + L775A + D801G + K875T + N892Y | | | | |
| E229S + I234V + V3521 + N672D + G753E + S754E + A769D + | 35 | 90 | 168 | 147 |
| L775A + D801G + K875T + N892Y | | | | |
| T18D + E229S + N672D + G753E + S754E + A769D + L775A + | 35 | 90 | 168 | 149 |
| D801G + K875T + N892Y + N1008D | | | | |
| A159P + E229S + I234V + N672D + G753E + S754E + A769D + | 35 | 90 | 168 | 149 |
| L775A + D801G + K875T + N892Y + N1008D | | | | |
| E229S + D458S + T631N + N672D + G753E + S754E + A769D + | 35 | 90 | 168 | 149 |
| L775A + D801G + K875T + N892Y | | | | |
| Q109R + E229S + T631N + N672D + P752R + G753E + S754E + | 35 | 90 | 149 | 150 |
| A769D + L775A + D801G + K875T + N892Y + T923H | | | | |
| E229S + S635E + T649K + 1656V + N672D + I703L + G753E + | 35 | 90 | 168 | 150 |
| S754E + A769D + L775A + D801G + K875T + N892Y | | | | |
| Q109R + E229S + N672D + G753E + S754E + A769E + L775A + | 35 | 90 | 166 | 152 |
| D801G + K875T + N892Y | | | | |
| Q109R + A159P + E229S + N672D + P752R + G753E + S754E + | 35 | 90 | 168 | 152 |
| A769T + L775A + D801G + K875T + N892Y | | | | |
| E229S + N672D + P752K + G753E + A769D + L775A + D801G + | 35 | 90 | 167 | 154 |
| K875T + N892Y | | | | |
| E229S + S635E + T649K + I656V + N672D + G753E + A769D + | 35 | 90 | 168 | 155 |
| L775A + D801G + K875T + N892Y + A911V | | | | |
| E229S + D458S + K567R + T631E + N672D + G753E + S754E + | 35 | 90 | 168 | 155 |
| A769D + L775A + D801G + K875T + N892Y | | | | |
| E229S + S635E + T649K + 1656V + N672D + I703L + G753E + | 35 | 90 | 168 | 158 |
| S754E + A769D + L775A + D801G + K875T + N892Y + N1008D | | | | |
| E229S + N440K + S582K + N672D + G753E + S754E + A769D + | 35 | 90 | 168 | 158 |
| L775A + D801G + K875T + N892Y + N1008D | | | | |
| Q109R + A159P + E229S + N672D + G753E + S754E + A769T + | 35 | 90 | 168 | 159 |
| L775A + D801G + A843P + K875T + N892Y | | | | |
| E229S + S635E + T649K + I656V + N672D + G753E + S754E + | 35 | 90 | 168 | 160 |
| A769D + L775A + D801G + K875T + N892Y + K1016T | | | | |
| E229S + S635E + N672D + G753E + S754E + A769D + L775A + | 35 | 90 | 168 | 160 |
| D801G + K875T + N892Y | | | | |
| E229S + N672D + P752R + G753E + S754E + A769D + L775A + | 35 | 90 | 167 | 160 |
| D801G + K875T + N892Y | | | | |
| E229S + N672D + G753E + S754E + A769D + L775A + D801G + | 35 | 90 | 168 | 161 |
| K875T + N892Y | | | | |
| E229S + D458S + N672D + G753E + S754E + A769D + L775A + | 35 | 90 | 149 | 162 |
| V800P + D801G + K875T + N892Y + N1008D | | | | |
| E229S + I234V + N672D + G753E + S754E + A769D + L775A + | 35 | 90 | 168 | 162 |
| D801G + K875T + N892Y | | | | |
| E229S + N672D + G753E + S754E + A769D + L775A + D801G + | 35 | 90 | 168 | 166 |
| K875T + N892Y | | | | |
| E229N + S635E + T649K + I656V + N672D + A769D + L775A + | 35 | 90 | 70 | 166 |
| D801G + K875T + N892Y + N991D | | | | |
| Q109R + A159P + E229S + N672D + P752K + G753E + S754E + | 35 | 90 | 168 | 167 |
| A769T + L775A + D801G + K875T + N892Y | | | | |
| E229S + S635E + T649K + N672D + G753E + S754E + A769D + | 35 | 90 | 168 | 167 |
| L775A + D801G + K875T + N892Y + N1008D | | | | |
| E229S + S635E + T649K + I656V + N672D + P752K + G753E + | 35 | 90 | 168 | 167 |
| S757D + A769D + L775A + D801G + K875T + N892Y + N1008D | | | | |
| Q109R + E229S + N672D + G753E + S754E + A769T + L775A + | 35 | 90 | 166 | 167 |
| D801G + A843P + K875T + N892Y | | | | |
| E229S + V3521 + S635E + T649K + 1656V + N672D + I703L + | 35 | 90 | 168 | 168 |
| G753E + A769D + L775A + D801G + K875T + N892Y + N1008D | | | | |
| Q109R + A159P + E229S + S635E + T649K + N672D + G738L + | 35 | 90 | 168 | 168 |
| G753E + S754E + S757D + A769T + L775A + D801G + K875T + | | | | |
| N892Y | | | | |
| E229S + N440K + S582K + A624E + N672D + G753E + S754E + | 35 | 90 | 168 | 169 |
| A769D + L775A + D801G + K875T + N892Y | | | | |
| T18D + A159P + E229S + I234V + N672D + G753E + S754E + | 35 | 90 | 168 | 173 |
| A769D + L775A + D801G + K875T + N892Y | | | | |
| E229S + D458S + N672D + G753E + S754E + A769D + L775A + | 35 | 90 | 168 | 174 |
| D801G + K875T + N892Y | | | | |
| A190Q + E229S + P752K + G753E + S757D + A769D + L775A + | 35 | 90 | 168 | 175 |
| D801G + K875T + N892Y | | | | |
| E229S + N440K + S582K + A624E + T631N + N672D + G753E + | 35 | 90 | 168 | 175 |
| S754E + A769D + L775A + D801G + K875T + N892Y | | | | |
| E229S + D458S + A492L + T631N + N672D + G753E + S754E + | 35 | 90 | 168 | 176 |
| A769D + L775A + D801G + K875T + N892Y | | | | |
| E229S + N672D + G753E + S754E + A769D + L775A + D801G + | 35 | 90 | 168 | 176 |
| K875T + N892Y | | | | |
| Q109R + A159P + E229S + A624E + A626G + S635E + T649K + | 35 | 90 | 168 | 176 |
| N672D + G753E + S754E + S757D + A769T + L775A + D801G + | | | | |
| K875T + N892Y | | | | |
| E229S + N440K + S582K + N672D + P752R + G753E + S754E + | 35 | 90 | 168 | 178 |
| A769D + L775A + D801G + K875T + N892Y + N1008D | | | | |
| E229S + D458S + T631N + S635E + T649K + N672D + G753E + | 35 | 90 | 168 | 178 |
| S754E + A769D + L775A + D801G + K875T + N892Y | | | | |
| E229S + S582K + N672D + G753E + S754E + A769D + L775A + | 35 | 90 | 168 | 179 |
| D801G + K875T + N892Y | | | | |
| E229S + I234V + A492L + N672D + G753E + S754E + A769D + | 35 | 90 | 168 | 181 |
| L775A + D801G + K875T + N892Y | | | | |
| Q109R + E229S + T631N + N672D + P752R + G753E + S754E + | 35 | 90 | 167 | 182 |
| S757D + A769T + L775A + D801G + K875T + N892Y | | | | |
| E229S + D458S + S582K + N672D + G753E + S754E + A769D + | 35 | 90 | 168 | 183 |
| L775A + D801G + K875T + N892Y + N1008D | | | | |
| A159P + E229S + S635E + T649K + 1656V + N672D + I703L + | 35 | 90 | 168 | 183 |
| G753E + S754E + A769D + L775A + D801G + K875T + N892Y + | | | | |
| N1008D | | | | |
| Q109R + E229S + T631N + N672D + P752R + G753E + S754E + | 35 | 90 | 168 | 188 |
| A769D + L775A + D801G + A843P + K875T + N892Y | | | | |
| Q109R + E229S + T631N + N672D + P752R + G753E + S754E + | 35 | 90 | 149 | 188 |
| S757D + A769D + L775A + D801G + K875T + N892Y | | | | |
| Q109R + A159P + E229S + S635E + T649K + N672D + I703L + | 35 | 90 | 149 | 188 |
| G753E + S754E + S757D + A769T + L775A + D801G + K875T + | | | | |
| N892Y | | | | |
| Q89Y + E229S + N672D + G753E + S754E + A769D + L775A + | 35 | 90 | 168 | 188 |
| D801G + K875T + N892Y | | | | |
| E229S + S635E + T649K + I656V + N672D + P752K + G753E + | 35 | 90 | 168 | 188 |
| A769D + L775A + D801G + A843P + K875T + N892Y | | | | |
| E229S + A492L + S635E + T649K + I656V + N672D + G753E + | 35 | 90 | 168 | 189 |
| A769D + L775A + D801G + K875T + N892Y | | | | |
| E229S + D458S + S582K + A624E + N672D + G753E + S754E + | 35 | 90 | 168 | 189 |
| A769D + L775A + D801G + K875T + N892Y | | | | |
| E229S + D458S + T631N + S635E + N672D + G753E + S754E + | 35 | 90 | 168 | 189 |
| A769D + L775A + D801G + K875T + N892Y | | | | |
| E229S + S635E + T649K + I656V + N672D + P752K + G753E + | 35 | 90 | 168 | 193 |
| A769D + L775A + V800P + D801G + K875T + N892Y + N1008D | | | | |
| E229S + D458S + N672D + G753E + S754E + A769D + L775A + | 35 | 90 | 149 | 193 |
| D801G + K875T + N892Y + N1008D | | | | |
| T18D + E229S + N440K + N672D + G753E + S754E + A769D + | 35 | 90 | 168 | 194 |
| L775A + D801G + K875T + N892Y | | | | |
| A159P + E229S + N440K + N672D + G753E + S754E + A769D + | 35 | 90 | 168 | 196 |
| L775A + D801G + K875T + N892Y | | | | |
| Q109R + A159P + E229S + S635E + T649K + N672D + P752R + | 35 | 90 | 168 | 197 |
| G753E + S754E + A769D + L775A + D801G + K875T + N892Y | | | | |
| E229S + I234V + S635E + T649K + I656V + N672D + G753E + | 35 | 90 | 168 | 197 |
| A769D + L775A + D801G + K875T + N892Y | | | | |
| Q109R + E229S + T631N + N672D + P752R + G753E + S754E + | 35 | 90 | 149 | 197 |
| A769D + L775A + D801G + K875T + N892Y + N1008D | | | | |
| E229S + D458S + T631E + N672D + G753E + S754E + A769D + | 35 | 90 | 168 | 200 |
| L775A + D801G + K875T + N892Y | | | | |
| A190Q + E229S + S635E + T649K + 1656V + N672D + I703L + | 35 | 90 | 168 | 200 |
| G753E + S754E + A769D + L775A + V800P + D801G + K875T + | | | | |
| N892Y + N1008D | | | | |
| E229S + D458S + N672D + P752R + G753E + S754E + A769D + | 35 | 90 | 168 | 200 |
| L775A + D801G + K875T + N892Y | | | | |
| A190Q + E229S + T631E + S635E + T649K + 1656V + N672D + | 35 | 90 | 168 | 203 |
| I703L + G753E + S754E + A769D + L775A + D801G + K875T + | | | | |
| N892Y + N1008D | | | | |
| E229S + N440K + A492L + S582K + A624E + N672D + G753E + | 35 | 90 | 168 | 203 |
| S754E + A769D + L775A + D801G + K875T + N892Y | | | | |
| E229S + D458S + K567R + T631N + N672D + G753E + S754E + | 35 | 90 | 168 | 203 |
| A769D + L775A + D801G + K875T + N892Y | | | | |
| L46D + E229S + K360R + S578K + N672D + G753E + S754E + | 35 | 90 | 168 | 203 |
| A769D + L775A + D801G + K875T + N892Y | | | | |
| E229S + S582K + S635E + T649K + 1656V + N672D + I703L + | 35 | 90 | 168 | 204 |
| G753E + S754E + A769D + L775A + D801G + K875T + N892Y + | | | | |
| N1008D | | | | |
| Q109R + A159P + E229S + S635Q + T649K + G753E + S754E + | 35 | 90 | 167 | 204 |
| S757D + A769D + L775A + D801G + K875T + N892Y | | | | |
| T18D + E229S + S635E + T649K + I656V + N672D + G753E + | 35 | 90 | 149 | 205 |
| S754E + A769D + L775A + D801G + K875T + N892Y | | | | |
| Q109R + A159P + E229S + G753E + S754E + S757D + A769D + | 35 | 90 | 168 | 206 |
| L775A + D801G + K875T + N892Y | | | | |
| E229S + N672D + G753E + S754E + A769D + L775A + D801G + | 35 | 90 | 168 | 206 |
| K875T + N892Y | | | | |
| E229N + T631N + N672D + P752K + G753E + A769D + L775A + | 35 | 90 | 149 | 207 |
| D801G + K875T + N892Y + A911V | | | | |
| Q109R + E229S + T631N + S635E + T649K + N672D + P752R + | 35 | 90 | 149 | 209 |
| G753E + S754E + A769D + L775A + D801G + K875T + N892Y | | | | |
| E229S + D458S + K567R + N672D + M728V + G753E + S754E + | 35 | 90 | 168 | 209 |
| A769D + L775A + D801G + K875T + N892Y | | | | |
| E229S + I234V + S582K + N672D + G753E + S754E + A769D + | 35 | 90 | 168 | 212 |
| L775A + D801G + K875T + N892Y | | | | |
| E229S + N440K + S582K + T631N + N672D + G753E + S754E + | 35 | 90 | 168 | 213 |
| A769D + L775A + D801G + K875T + N892Y + N1008D | | | | |
| A190Q + E229S + I234V + S582K + N672D + G753E + S754E + | 35 | 90 | 168 | 215 |
| A769D + L775A + D801G + K875T + N892Y | | | | |
| A190Q + E229S + I234V + A624E + N672D + G753E + S754E + | 35 | 90 | 168 | 216 |
| A769D + L775A + D801G + K875T + N892Y | | | | |
| E229S + N440K + S582K + A624E + S635E + N672D + G753E + | 35 | 90 | 168 | 216 |
| S754E + A769D + L775A + D801G + K875T + N892Y | | | | |
| E229S + S635E + T649K + I656V + N672D + G753E + S754E + | 35 | 90 | 168 | 218 |
| A769D + L775A + D801G + K875T + N892Y | | | | |
| S100D + E229S + S635E + T649K + 1656V + N672D + I703L + | 35 | 90 | 168 | 219 |
| G753E + A769D + L775A + D801G + A843P + K875T + N892Y + | | | | |
| N1008D | | | | |
| A190Q + E2295 + S635E + T649K + 1656V + N672D + I703L + | 35 | 90 | 168 | 224 |
| P752R + G753E + S754E + A769D + L775A + D801G + K875T + | | | | |
| N892Y + N1008D | | | | |
| Q89Y + E2295 + N440K + 5582K + A624E + N672D + G753E + | 35 | 90 | 168 | 225 |
| S754E + A769D + L775A + D801G + K875T + N892Y | | | | |
| A190Q + E2295 + S635E + T649K + 1656V + N672D + I703L + | 35 | 90 | 168 | 231 |
| G753E + S754E + 5757D + A769D + L775A + D801G + K875T + | | | | |
| N892Y + N1008D | | | | |
| E2295 + I234V + N672D + G753E + S754E + A769D + L775A + | 35 | 90 | 149 | 235 |
| D801G + A843P + K875T + N892Y | | | | |
| E2295 + S635E + T649K + I656V + N672D + G753E + S754E + | 35 | 90 | 168 | 236 |
| S757D + A769D + L775A + D801G + K875T + N892Y | | | | |
| T18D + E229S + S582K + N672D + G753E + S754E + A769D + | 35 | 90 | 168 | 239 |
| L775A + D801G + K875T + N892Y | | | | |
| E2295 + D4585 + T631N + N672D + G753E + S754E + S757D + | 35 | 90 | 168 | 239 |
| A769D + L775A + D801G + K875T + N892Y | | | | |
| E2295 + S635E + T649K + I656V + N672D + P752R + G753E + | 35 | 90 | 168 | 239 |
| S754E + A769D + L775A + D801G + K875T + N892Y + N1008D | | | | |
| E2295 + N440K + 5582K + A624E + N672D + G753E + S754E + | 35 | 90 | 168 | 242 |
| A769D + L775A + V800P + D801G + K875T + N892Y | | | | |
| A190Q + E2295 + D4585 + T631N + N672D + G753E + S754E + | 35 | 90 | 168 | 245 |
| A769D + L775A + D801G + K875T + N892Y | | | | |
| E2295 + K360G + D4585 + S582K + N672D + G753E + S754E + | 35 | 90 | 168 | 251 |
| A769D + L775A + D801G + K875T + N892Y + N1008D | | | | |
| S100D + E2295 + S635E + T649K + 1656V + N672D + G753E + | 35 | 90 | 149 | 253 |
| S754E + A769D + L775A + D801G + K875T + N892Y | | | | |
| A190Q + E229S + S635E + T649K + 1656V + N672D + G753E + | 35 | 90 | 149 | 261 |
| S754E + A769D + L775A + D801G + K875T + N892Y | | | | |
| S100D + A190Q + E229S + S635E + T649K + 1656V + N672D + | 35 | 90 | 168 | 263 |
| I703L + G753E + S754E + A769D + L775A + D801G + K875T + | | | | |
| N892Y + N1008D | | | | |
| E229S + D458S + K567R + S635E + N672D + G753E + S754E + | 35 | 90 | 168 | 266 |
| A769D + L775A + D801G + K875T + N892Y | | | | |
| E229S + D458S + N672D + G753E + S754E + A769D + L775A + | 35 | 90 | 149 | 281 |
| D801G + A843P + K875T + N892Y | | | | |
| E229S + T631N + N672D + I703L + P752K + G753E + A769D + | 35 | 90 | 149 | 284 |
| L775A + D801G + K875T + N892Y | | | | |
| E229S + N440K + S582K + N672D + G753E + S754E + A769D + | 35 | 90 | 149 | 296 |
| L775A + D801G + A843P + K875T + N892Y + N1008D | | | | |

TABLE 32
Half-life of purified variants: Temperature (T) 35° C., detergent concentration 95%

| | | | Incubation | Half- |
|---|---|---|---|---|
| | T | Detergent | time | life |
| Mutations relative to SEQ ID NO: 6 | (° C.) | (%) | (h) | (h) |
| No mutations (Wild-type) | 35 | 95 | 1 | <0.2 |
| E229S + K360G + D458S + S582K + T631N + S635E + N672D + | 35 | 95 | 168 | 40 |
| I703L + M728V + G753E + S754E + S757D + A769D + L775A + | | | | |
| D801G + K875T + N892Y | | | | |
| E229S + D458S + S582K + T631N + S635E + N672D + M728V + | 35 | 95 | 168 | 27 |
| G753E + S754E + S757D + A769D + L775A + D777R + V800P + | | | | |
| D801G + K875T + N892Y | | | | |
| E229S + D458S + S582K + T631N + S635E + N672D + M728V + | 35 | 95 | 168 | 39 |
| G753E + S754E + S757D + A769D + L775A + D801G + A843P + | | | | |
| K875T + N892Y | | | | |
| E229S + D458S + S582K + T631N + S635E + N672D + M728V + | 35 | 95 | 168 | 46 |
| G753E + S754E + S757D + A769D + L775A + D801G + A843P + | | | | |
| K875T + N892Y | | | | |
| E229S + D458S + S582K + T631N + S635E + T664K + N672D + | 35 | 95 | 168 | 41 |
| M728V + G753E + S754E + S757D + A769D + L775A + D801G + | | | | |
| K875T + N892Y | | | | |
| E229S + V352I + D458S + S582K + T631N + S635E + N672D + | 35 | 95 | 168 | 43 |
| M728V + G753E + S754E + S757D + A769D + L775A + D801G + | | | | |
| K875T + N892Y + K1016T | | | | |
| S100D + E229S + K360G + A624E + S635E + T649K + N672D + | 35 | 95 | 168 | 54 |
| G753E + S754E + A769D + L775A + D777K + D801G + K875T + | | | | |
| N892Y + K1016T | | | | |
| S100D + E2295 + V352I + K360G + D458S + A624E + S635E + | 35 | 95 | 168 | 42 |
| T649K + I656V + N672D + G753E + S754R + S757D + A769D + | | | | |
| L775A + D777K + D801G + K875T + N892Y + K1016T | | | | |
| E2295 + A624E + S635E + T649K + I656V + N672D + L748T + | 35 | 95 | 168 | 37 |
| G753E + S754R + S757D + A769D + L775A + D777K + D801G + | | | | |
| K875T + N892Y | | | | |
| E2295 + S582K + A624E + S635E + T649K + I656V + N672D + | 35 | 95 | 168 | 38 |
| G753E + S754R + S757D + A769D + L775A + D777K + V800P + | | | | |
| D801G + K875T + N892Y | | | | |
| E2295 + A624E + S635E + T649K + N672D + G753E + S754E + | 35 | 95 | 168 | 45 |
| A769D + L775A + D777K + D801G + K875T + N892Y | | | | |
| E2295 + A624E + S635E + T649K + I656V + N672D + G753E + | 35 | 95 | 168 | 55 |
| S754R + S757D + A769D + L775A + D777R + D801G + A843P + | | | | |
| K875T + N892Y | | | | |
| E2295 + D4585 + K567R + S582K + S635E + N672D + M728V + | 35 | 95 | 168 | 23 |
| G753E + S754R + S757D + A769D + L775A + D777K + D801G + | | | | |
| K875T + N892Y + K1016T | | | | |
| E229S + D458S + K567R + S582K + S635E + N672D + G753E + | 35 | 95 | 168 | 26 |
| S754E + A769D + L775A + D777R + V800P + D801G + K875T + | | | | |
| N892Y | | | | |
| E229S + D458S + K567R + S582K + S635E + N672D + G753E + | 35 | 95 | 168 | 29 |
| S754E + A769D + L775A + D777R + D801G + K875T + N892Y + | | | | |
| N1008D | | | | |
| E229S + D458S + K567R + S582K + S635E + T649K + N672D + | 35 | 95 | 168 | 29 |
| G753E + S754E + A769D + L775A + D777K + D801G + K875T + | | | | |
| N892Y | | | | |
| E229S + N399K + D458S + A492H + K567R + S582K + S635E + | 35 | 95 | 168 | 61 |
| T649K + N672D + G753E + S754E + A769D + L775A + D777R + | | | | |
| D801G + K875T + N892Y | | | | |
| E229S + D458S + K567R + S582K + S635E + N672D + G753E + | 35 | 95 | 168 | 23 |
| S754E + S757D + A769D + L775A + D777R + D801G + K875T + | | | | |
| N892Y | | | | |
| E229S + D458S + K567R + S582K + S635E + T664K + N672D + | 35 | 95 | 168 | 25 |
| G753E + S754E + A769D + L775A + D777R + K792Y + D801G + | | | | |
| K875T + N892Y | | | | |
| E229S + D458S + K567R + S582K + S635E + T664K + N672D + | 35 | 95 | 168 | 25 |
| G753E + S754E + A769D + L775A + D777R + D801G + K875T + | | | | |
| N892Y | | | | |
| E229S + D458S + K567R + S582K + S635E + N672D + M728V + | 35 | 95 | 168 | 30 |
| G753E + S754E + A769D + L775A + D777R + D801G + K875T + | | | | |
| N892Y | | | | |
| S100D + E229S + K360G + D458S + S582K + A624E + N672D + | 35 | 95 | 168 | 60 |
| G753E + S754E + S757D + A769D + L775A + D801G + A843P + | | | | |
| K875T + N892Y + N1008D | | | | |
| S100D + E229S + K360G + D458S + S582K + N672D + G753E + | 35 | 95 | 168 | 50 |
| S754E + S757D + A769D + L775A + D801G + A843P + K875T + | | | | |
| N892Y + N1008D | | | | |
| S100D + E229S + K360G + D458S + S582K + S635E + N672D + | 35 | 95 | 168 | 51 |
| G753E + S754E + 5757D + A769D + L775A + V800P + D801G + | | | | |
| A843P + K875T + N892Y + N1008D | | | | |
| S100D + E229S + K360G + D458S + S582K + S635E + T649K + | 35 | 95 | 168 | 59 |
| N672D + G753E + S754E + S757D + A769D + L775A + D801G + | | | | |
| A843P + K875T + N892Y + N1008D | | | | |
| E2295 + S635E + T649R + N672D + P752R + G753E + S754E + | 35 | 95 | 168 | 37 |
| A769D + L775A + D777K + D801G + K875T + N892Y | | | | |
| E2295 + N672D + P752R + G753E + S754E + S757D + A769D + | 35 | 95 | 168 | 47 |
| L775A + D801G + A843P + K875T + N892Y | | | | |
| E2295 + S635E + T649K + I656V + N672D + G753E + A769D + | 35 | 95 | 168 | 24 |
| L775A + D801G + K875T + N892Y | | | | |
| S100D + E2295 + K360G + D4585 + S582K + N672D + G753E + | 35 | 95 | 168 | 45 |
| S754E + A769D + L775A + D801G + K875T + N892Y + N1008D | | | | |
| A190Q + E2295 + K360G + D4585 + S582K + N672D + G753E + | 35 | 95 | 168 | 41 |
| S754E + A769D + L775A + D801G + K875T + N892Y + N1008D | | | | |

TABLE 33
Half-life of purified variants: Temperature (T) 37° C., detergent concentration 90%

| | T | Detergent | Incubation | Half- |
|---|---|---|---|---|
| Mutations relative to SEQ ID NO: 6 | (° C.) | (%) | time (h) | life (h) |
| No mutations (Wild-type) | 37 | 90 | 1 | <0.2 |
| Q109R + A159P + E229S + V352I + S635E + | 37 | 90 | 120 | 29 |
| T649K + N672D + I703L + G753E + S754E + | | | | |
| S757D + A769T + L775A + D801G + K875T + | | | | |
| N892Y | | | | |
| E229N + T631N + N672D + G753E + S754E + | 37 | 90 | 120 | 40 |
| A769D + L775A + D801G + K875T + N892Y + | | | | |
| N1008D | | | | |
| E229S + N440K + S582K + A624E + S635E + | 37 | 90 | 120 | 53 |
| N672D + G753E + S754E + S757D + A769D + | | | | |
| L775A + D801G + A843P + K875T + N892Y | | | | |
| E229S + I234V + T631N + N672D + I703L + | 37 | 90 | 120 | 63 |
| P752K + G753E + A769D + L775A + D801G + | | | | |
| K875T + N892Y | | | | |
| Q109R + A159P + E229S + S582K + S635Q + | 37 | 90 | 120 | 64 |
| T649K + G753E + S754E + S757D + A769D + | | | | |
| L775A + D801G + K875T + N892Y | | | | |
| Q109R + A159P + A190Q + E229S + G753E + | 37 | 90 | 120 | 65 |
| S754E + S757D + A769D + L775A + D801G + | | | | |
| K875T + N892Y | | | | |
| A159P + E229S + S635E + T649K + I656V + | 37 | 90 | 120 | 67 |
| N672D + G753E + S754E + A769D + L775A + | | | | |
| V800P + D801G + K875T + N892Y | | | | |
| T18D + Q89Y + E229S + S635E + T649K + | 37 | 90 | 120 | 69 |
| I656V + N672D + G753E + S754E + A769D + | | | | |
| L775A + D801G + K875T + N892Y | | | | |
| T18D + A190Q + E229S + S635E + T649K + | 37 | 90 | 120 | 70 |
| I656V + N672D + G753E + S754E + A769D + | | | | |
| L775A + D801G + K875T + N892Y | | | | |
| Q109R + A159P + E229S + T631N + G753E + | 37 | 90 | 120 | 71 |
| S754E + S757D + A769D + L775A + D801G + | | | | |
| K875T + N892Y | | | | |
| A58L + E229S + N440K + S582K + T631N + | 37 | 90 | 120 | 71 |
| N672D + G753E + S754E + A769D + L775A + | | | | |
| D801G + K875T + N892Y + N1008D | | | | |
| E229S + S635E + T649K + I656V + N672D + | 37 | 90 | 120 | 72 |
| G753E + S754E + A769D + L775A + V800P + | | | | |
| D801G + K875T + N892Y | | | | |
| E229S + T631N + N672D + I703L + P752R + | 37 | 90 | 120 | 73 |
| G753E + A769D + L775A + D801G + K875T + | | | | |
| N892Y | | | | |
| T18D + Q109R + E229S + T631N + S635E + | 37 | 90 | 120 | 74 |
| T649K + N672D + P752R + G753E + S754E + | | | | |
| A769D + L775A + D801G + K875T + N892Y | | | | |
| A190Q + E229S + D458S + K567R + S635E + | 37 | 90 | 120 | 74 |
| N672D + G753E + S754E + A769D + L775A + | | | | |
| D801G + K875T + N892Y | | | | |
| Q109R + A159P + E229S + T631E + S635Q + | 37 | 90 | 120 | 77 |
| T649K + G753E + S754E + S757D + A769D + | | | | |
| L775A + D801G + K875T + N892Y | | | | |
| E229S + T631N + N672D + I703L + P752K + | 37 | 90 | 120 | 77 |
| G753E + A769D + L775A + D801G + K875T + | | | | |
| N892Y | | | | |
| E229S + T631N + N672D + I703L + P752K + | 37 | 90 | 120 | 79 |
| G753E + A769D + L775A + D801G + K875T + | | | | |
| N892Y + N1008D | | | | |
| Q89Y + E229S + D458S + T631N + N672D + | 37 | 90 | 120 | 80 |
| G753E + S754E + S757D + A769D + L775A + | | | | |
| D801G + K875T + N892Y | | | | |
| A190Q + E229S + D458S + T631N + N672D + | 37 | 90 | 120 | 80 |
| G753E + S754E + A769D + L775A + D801G + | | | | |
| K875T + N892Y | | | | |
| Q109R + E229S + T631N + N672D + M728V + | 37 | 90 | 120 | 82 |
| P752R + G753E + S754E + S757D + A769D + | | | | |
| L775A + D801G + K875T + N892Y | | | | |
| Q109R + A159P + E229S + T631N + S635Q + | 37 | 90 | 120 | 82 |
| T649K + G753E + S754E + S757D + A769D + | | | | |
| L775A + D801G + K875T + N892Y | | | | |
| Q109R + A159P + E229S + S635Q + T649K + | 37 | 90 | 120 | 82 |
| G753E + S754E + S757D + A769D + L775A + | | | | |
| D801G + K875T + N892Y | | | | |
| Q109R + A159P + E229S + S635E + T649K + | 37 | 90 | 120 | 83 |
| N672D + I703L + M728V + G753E + S754E + | | | | |
| S757D + A769T + L775A + D801G + K875T + | | | | |
| N892Y | | | | |
| E229S + D458S + T631N + N672D + G753E + | 37 | 90 | 120 | 83 |
| S754E + S757D + A769D + L775A + D801G + | | | | |
| K875T + N892Y | | | | |
| E229S + D458S + S582K + T631N + N672D + | 37 | 90 | 120 | 84 |
| M728V + G753E + S754E + S757D + A769D + | | | | |
| L775A + D801G + K875T + N892Y | | | | |
| Q109R + E229S + S582K + T631N + S635E + | 37 | 90 | 120 | 84 |
| T649K + N672D + P752R + G753E + S754E + | | | | |
| A769D + L775A + D801G + K875T + N892Y | | | | |
| A190Q + E229S + D458S + T631N + S635E + | 37 | 90 | 120 | 84 |
| N672D + G753E + S754E + A769D + L775A + | | | | |
| D801G + K875T + N892Y | | | | |
| E229S + T631N + N672D + I703L + P752R + | 37 | 90 | 120 | 84 |
| G753E + S754E + A769D + L775A + D801G + | | | | |
| K875T + N892Y | | | | |
| E229S + S635E + T649K + I656V + N672D + | 37 | 90 | 120 | 84 |
| P752K + G753E + S754E + A769D + L775A + | | | | |
| V800P + D801G + K875T + N892Y | | | | |
| L46D + E229S + T631N + N672D + I703L + | 37 | 90 | 120 | 84 |
| P752K + G753E + A769D + L775A + D801G + | | | | |
| K875T + N892Y | | | | |
| A190Q + E229S + R284G + S635E + T649K + | 37 | 90 | 120 | 85 |
| I656V + N672D + G753E + S754E + A769D + | | | | |
| L775A + V800P + D801G + K875T + N892Y | | | | |
| T18D + A190Q + E229S + D458S + T631N + | 37 | 90 | 120 | 85 |
| N672D + G753E + S754E + A769D + L775A + | | | | |
| D801G + K875T + N892Y | | | | |
| E229S + S635E + T649K + I656V + N672D + | 37 | 90 | 120 | 86 |
| G753E + S754E + A769D + L775A + V800P + | | | | |
| D801G + A843P + K875T + N892Y | | | | |
| E229S + I234V + N440K + S582K + A624E + | 37 | 90 | 120 | 86 |
| S635E + N672D + G753E + S754E + A769D + | | | | |
| L775A + D801G + K875T + N892Y | | | | |
| Q109R + A159P + E229S + S635E + T649K + | 37 | 90 | 120 | 86 |
| N672D + I703L + G753E + S754E + S757D + | | | | |
| A769T + L775A + V800P + D801G + K875T + | | | | |
| N892Y | | | | |
| Q109R + E229S + K360G + T631N + S635E + | 37 | 90 | 120 | 87 |
| T649K + N672D + P752R + G753E + S754E + | | | | |
| A769D + L775A + D801G + K875T + N892Y | | | | |
| E229S + S582K + T631N + N672D + I703L + | 37 | 90 | 120 | 87 |
| P752K + G753E + A769D + L775A + D801G + | | | | |
| K875T + N892Y | | | | |
| E229S + I234V + S582K + N672D + M728V + | 37 | 90 | 120 | 87 |
| G753E + S754E + A769D + L775A + D801G + | | | | |
| K875T + N892Y | | | | |
| E229S + A492L + S635E + T649K + I656V + | 37 | 90 | 120 | 87 |
| N672D + G753E + S754E + A769D + L775A + | | | | |
| V800P + D801G + K875T + N892Y | | | | |
| E229S + N440K + S582K + A624E + S635E + | 37 | 90 | 120 | 87 |
| N672D + G753E + S754E + A769D + L775A + | | | | |
| D777K + D801G + K875T + N892Y | | | | |
| L46D + E229S + N440K + S582K + T631N + | 37 | 90 | 120 | 88 |
| N672D + G753E + S754E + A769D + L775A + | | | | |
| D801G + K875T + N892Y + N1008D | | | | |
| Q109R + A159P + E229S + A624E + S635E + | 37 | 90 | 120 | 88 |
| T649K + N672D + I703L + G753E + S754E + | | | | |
| S757D + A769T + L775A + D801G + K875T + | | | | |
| N892Y | | | | |
| E229S + N672D + G753E + S754E + A769D + | 37 | 90 | 120 | 88 |
| L775A + D801G + K875T + N892Y | | | | |
| E229S + A492L + S635E + T649K + I656V + | 37 | 90 | 120 | 88 |
| N672D + G753E + A769D + L775A + D801G + | | | | |
| K875T + N892Y + N1008D | | | | |
| Q109R + A159P + E229S + T631N + S635E + | 37 | 90 | 120 | 88 |
| T649K + N672D + I703L + G753E + S754E + | | | | |
| S757D + A769T + L775A + D801G + K875T + | | | | |
| N892Y | | | | |
| T18D + S100D + E229S + S635E + T649K + | 37 | 90 | 120 | 88 |
| I656V + N672D + I703L + G753E + A769D + | | | | |
| L775A + D801G + A843P + K875T + N892Y + | | | | |
| N1008D | | | | |
| T18D + E229S + S582K + T664K + N672D + | 37 | 90 | 120 | 89 |
| G753E + S754E + A769D + L775A + D801G + | | | | |
| K875T + N892Y | | | | |
| Q109R + A159P + E229S + T631N + S635E + | 37 | 90 | 120 | 89 |
| T649K + N672D + P752R + G753E + S754E + | | | | |
| A769D + L775A + D801G + K875T + N892Y | | | | |
| A58L + E229S + S635E + T649K + I656V + | 37 | 90 | 120 | 91 |
| N672D + G753E + S754E + S757D + A769D + | | | | |
| L775A + D801G + K875T + N892Y | | | | |
| E229S + V352I + D458S + K567R + S635E + | 37 | 90 | 120 | 92 |
| N672D + G753E + S754E + A769D + L775A + | | | | |
| D801G + K875T + N892Y | | | | |
| E229S + S635E + T649K + I656V + N672D + | 37 | 90 | 120 | 92 |
| P752R + G753E + S754E + A769D + L775A + | | | | |
| D801G + K875T + N892Y + N1008D + K1016T | | | | |
| T18D + S100D + E229S + S635E + T649K + | 37 | 90 | 120 | 93 |
| I656V + N672D + G753E + S754E + A769D + | | | | |
| L775A + D801G + K875T + N892Y | | | | |
| T18D + E229S + A492L + S635E + T649K + | 37 | 90 | 120 | 93 |
| I656V + N672D + G753E + A769D + L775A + | | | | |
| D801G + K875T + N892Y | | | | |
| E229S + N440K + D458S + S582K + A624E + | 37 | 90 | 120 | 93 |
| S635E + N672D + G753E + S754E + A769D + | | | | |
| L775A + D801G + K875T + N892Y | | | | |
| T18D + E229S + Q372H + S582K + N672D + | 37 | 90 | 120 | 93 |
| G753E + S754E + A769D + L775A + D801G + | | | | |
| K875T + N892Y | | | | |
| E229S + D458S + T631N + N672D + G753E + | 37 | 90 | 120 | 93 |
| S754E + S757D + A769D + L775A + D801G + | | | | |
| K875T + N892Y | | | | |
| S100D + E229S + N440K + S582K + A624E + | 37 | 90 | 120 | 93 |
| S635E + N672D + G753E + S754E + A769D + | | | | |
| L775A + D801G + K875T + N892Y | | | | |
| S100D + E229S + S635E + T649K + I656V + | 37 | 90 | 120 | 94 |
| N672D + G753E + S754E + A769D + L775A + | | | | |
| V800P + D801G + K875T + N892Y | | | | |
| E229S + I234V + S582K + N672D + I703L + | 37 | 90 | 120 | 95 |
| G753E + S754E + A769D + L775A + D801G + | | | | |
| K875T + N892Y | | | | |
| T18D + E229S + S582K + N672D + G753E + | 37 | 90 | 120 | 95 |
| S754E + A769D + L775A + D801G + E856D + | | | | |
| K875T + N892Y | | | | |
| Q109R + A159P + E229S + S635Q + T649K + | 37 | 90 | 120 | 95 |
| G753E + S754E + S757D + A769D + L775A + | | | | |
| D801G + A843P + K875T + N892Y | | | | |
| E229S + T631N + N672D + I703L + P752K + | 37 | 90 | 120 | 96 |
| G753E + A769D + L775A + D801G + K875T + | | | | |
| N892Y + K1016T | | | | |
| A159P + E229S + A492L + S635E + T649K + | 37 | 90 | 120 | 96 |
| I656V + N672D + G753E + A769D + L775A + | | | | |
| D801G + K875T + N892Y | | | | |
| E229S + T631N + N672D + I703L + P752K + | 37 | 90 | 120 | 96 |
| G753E + A769D + L775A + D801G + A843P + | | | | |
| K875T + N892Y | | | | |
| E229S + N440K + S582K + T631N + S635E + | 37 | 90 | 120 | 96 |
| T649K + N672D + G753E + S754E + A769D + | | | | |
| L775A + D801G + K875T + N892Y + N1008D | | | | |
| E229S + D458S + K567R + S582K + S635E + | 37 | 90 | 120 | 96 |
| N672D + G753E + S754E + A769D + L775A + | | | | |
| D777R + D801G + K875T + N892Y | | | | |
| T18D + E229S + S582K + N672D + G753E + | 37 | 90 | 120 | 97 |
| S754E + A769D + L775A + D801G + K875T + | | | | |
| N892Y + I900G | | | | |
| L46D + E229S + K360G + D458S + S582K + | 37 | 90 | 120 | 97 |
| N672D + G753E + S754E + A769D + L775A + | | | | |
| D801G + K875T + N892Y + N1008D | | | | |
| T18D + E229S + S582K + N672D + G753E + | 37 | 90 | 120 | 97 |
| S754E + A769D + L775A + D801G + K819T + | | | | |
| K875T + N892Y | | | | |
| S100D + E229S + K360G + D458S + S582K + | 37 | 90 | 120 | 97 |
| N672D + G753E + S754E + A769D + L775A + | | | | |
| D777R + V800P + D801G + K875T + N892Y + | | | | |
| N1008D | | | | |
| E229S + D458S + K567R + S582K + S635E + | 37 | 90 | 120 | 98 |
| N672D + G753E + S754E + A769D + L775A + | | | | |
| D801G + K875T + N892Y | | | | |
| E229S + A624E + S635E + T649K + I656V + | 37 | 90 | 120 | 100 |
| N672D + P752R + G753E + S754E + A769D + | | | | |
| L775A + D801G + K875T + N892Y + N1008D | | | | |
| E229S + N440K + K567R + S582K + A624E + | 37 | 90 | 120 | 100 |
| S635E + N672D + G753E + S754E + A769D + | | | | |
| L775A + D801G + K875T + N892Y | | | | |
| T18D + E229S + S582K + N672D + G753E + | 37 | 90 | 120 | 100 |
| S754E + P764V + A769D + L775A + D801G + | | | | |
| K875T + N892Y | | | | |
| A190Q + E229S + A492L + S635E + T649K + | 37 | 90 | 120 | 100 |
| I656V + N672D + G753E + A769D + L775A + | | | | |
| D801G + K875T + N892Y | | | | |
| E229S + N440K + S582K + T631E + N672D + | 37 | 90 | 120 | 100 |
| G753E + S754E + A769D + L775A + D801G + | | | | |
| A843P + K875T + N892Y + N1008D | | | | |
| T18D + Q89Y + E229S + S582K + N672D + | 37 | 90 | 120 | 100 |
| G753E + S754E + A769D + L775A + D801G + | | | | |
| K875T + N892Y | | | | |
| S100D + Q109R + A159P + E229S + S635E + | 37 | 90 | 120 | 102 |
| T649K + N672D + I703L + G753E + S754E + | | | | |
| S757D + A769T + L775A + D801G + K875T + | | | | |
| N892Y | | | | |
| Q109R + A159P + E229S + S635E + T649K + | 37 | 90 | 120 | 102 |
| N672D + P752R + G753E + S754E + A769D + | | | | |
| L775A + D801G + K875T + N892Y | | | | |
| T18D + E229S + S582K + N672D + G753E + | 37 | 90 | 120 | 102 |
| S754E + A769D + L775A + D801G + P867S + | | | | |
| K875T + N892Y | | | | |
| E229S + K567R + S635E + T649K + I656V + | 37 | 90 | 120 | 102 |
| N672D + P752K + G753E + A769D + L775A + | | | | |
| D801G + A843P + K875T + N892Y | | | | |
| E229S + D458S + S635E + T649K + I656V + | 37 | 90 | 120 | 104 |
| N672D + G753E + S754E + A769D + L775A + | | | | |
| V800P + D801G + K875T + N892Y | | | | |
| E229S + S635E + T649K + I656V + N672D + | 37 | 90 | 120 | 104 |
| P752K + G753E + S754E + A769D + L775A + | | | | |
| D801G + A843P + K875T + N892Y | | | | |
| Q109R + E229S + T631N + S635E + N672D + | 37 | 90 | 120 | 105 |
| P752R + G753E + S754E + S757D + A769D + | | | | |
| L775A + D801G + K875T + N892Y | | | | |
| E229S + A624E + S635E + T649K + I656V + | 37 | 90 | 120 | 106 |
| N672D + G753E + S754R + S757D + A769D + | | | | |
| L775A + D777R + D801G + K875T + N892Y | | | | |
| E229S + T631N + S635E + T649K + I656V + | 37 | 90 | 120 | 106 |
| N672D + P752K + G753E + S757D + A769D + | | | | |
| L775A + D801G + A843P + K875T + N892Y | | | | |
| E229S + D458S + S635E + T649K + I656V + | 37 | 90 | 120 | 106 |
| N672D + G753E + S754R + S757D + A769D + | | | | |
| L775A + D801G + K875T + N892Y | | | | |
| E229S + D458S + T631N + N672D + G753E + | 37 | 90 | 120 | 107 |
| S754E + S757D + A769D + L775A + D777K + | | | | |
| D801G + K875T + N892Y | | | | |
| E229S + A624E + S635E + T649K + I656V + | 37 | 90 | 120 | 107 |
| N672D + G753E + S754R + S757D + A769D + | | | | |
| L775A + D801G + K875T + N892Y | | | | |
| T18D + E229S + S582K + N672D + G753E + | 37 | 90 | 120 | 107 |
| S754E + S757D + A769D + L775A + D801G + | | | | |
| K875T + N892Y | | | | |
| E229S + I234V + D458S + S582K + N672D + | 37 | 90 | 120 | 108 |
| G753E + S754E + A769D + L775A + D801G + | | | | |
| K875T + N892Y | | | | |
| Q109R + E229S + K567R + T631N + N672D + | 37 | 90 | 120 | 109 |
| P752R + G753E + S754E + S757D + A769D + | | | | |
| L775A + D801G + K875T + N892Y | | | | |
| S100D + A190Q + E229S + D458S + T631N + | 37 | 90 | 120 | 109 |
| N672D + G753E + S754E + A769D + L775A + | | | | |
| D801G + K875T + N892Y | | | | |
| S100D + E229S + S582K + S635E + T649K + | 37 | 90 | 120 | 109 |
| I656V + N672D + I703L + G753E + A769D + | | | | |
| L775A + D801G + A843P + K875T + N892Y + | | | | |
| N1008D | | | | |
| E229S + T631N + N672D + I703L + P752K + | 37 | 90 | 120 | 109 |
| G753E + S754E + A769D + L775A + D801G + | | | | |
| K875T + N892Y | | | | |
| A190Q + E229S + I234V + S582K + N672D + | 37 | 90 | 120 | 110 |
| G753E + S754E + A769D + L775A + D801G + | | | | |
| A843P + K875T + N892Y | | | | |
| E229S + D458S + T631E + N672D + G753E + | 37 | 90 | 120 | 111 |
| S754E + A769D + L775A + D777R + D801G + | | | | |
| K875T + N892Y | | | | |
| T18D + E229S + D458S + K567R + S582K + | 37 | 90 | 120 | 111 |
| S635E + N672D + G753E + S754E + A769D + | | | | |
| L775A + D801G + K875T + N892Y | | | | |
| E229S + N440K + D458S + S582K + T631N + | 37 | 90 | 120 | 111 |
| N672D + G753E + S754E + A769D + L775A + | | | | |
| D801G + K875T + N892Y + N1008D | | | | |
| E229S + D458S + K567R + S582K + T631N + | 37 | 90 | 120 | 112 |
| S635E + N672D + G753E + S754E + A769D + | | | | |
| L775A + D801G + K875T + N892Y | | | | |
| E229S + K360G + D458S + S582K + N672D + | 37 | 90 | 120 | 112 |
| P752R + G753E + S754E + A769D + L775A + | | | | |
| D801G + K875T + N892Y + N1008D | | | | |
| E229S + D458S + S582K + T631N + N672D + | 37 | 90 | 120 | 112 |
| G753E + S754E + S757D + A769D + L775A + | | | | |
| D801G + K875T + N892Y | | | | |
| E229S + K360G + D458S + S582K + S635E + | 37 | 90 | 120 | 112 |
| N672D + G753E + S754E + A769D + L775A + | | | | |
| D801G + K875T + N892Y + N1008D | | | | |
| E229S + S582K + S635E + T649K + I656V + | 37 | 90 | 120 | 112 |
| N672D + P752K + G753E + A769D + L775A + | | | | |
| D801G + A843P + K875T + N892Y | | | | |
| E229S + D458S + K567R + S582K + S635E + | 37 | 90 | 120 | 112 |
| N672D + G753E + S754E + A769D + L775A + | | | | |
| D801G + K875T + N892Y + K1016T | | | | |
| E229S + T631N + N672D + P752R + G753E + | 37 | 90 | 120 | 113 |
| S754E + A769D + L775A + D801G + A843P + | | | | |
| K875T + N892Y | | | | |
| E229S + N440K + S582K + N672D + P752R + | 37 | 90 | 120 | 113 |
| G753E + S754E + A769D + L775A + D801G + | | | | |
| A843P + K875T + N892Y + N1008D | | | | |
| S100D + E229S + S635E + T649K + I656V + | 37 | 90 | 120 | 114 |
| N672D + I703L + G753E + A769D + L775A + | | | | |
| D777R + D801G + A843P + K875T + N892Y + | | | | |
| N1008D | | | | |
| E229S + D458S + K567R + S582K + S635E + | 37 | 90 | 120 | 115 |
| N672D + G753E + S754E + A769D + L775A + | | | | |
| D777K + D801G + K875T + N892Y | | | | |
| E229S + D458S + K567R + T631N + S635E + | 37 | 90 | 120 | 115 |
| N672D + G753E + S754E + A769D + L775A + | | | | |
| D801G + K875T + N892Y | | | | |
| A190Q + E229S + D458S + T631N + N672D + | 37 | 90 | 120 | 115 |
| G753E + S754E + A769D + L775A + D801G + | | | | |
| K875T + N892Y | | | | |
| E229S + N440K + S582K + A624E + S635E + | 37 | 90 | 120 | 115 |
| N672D + G753E + S754E + S757D + A769D + | | | | |
| L775A + D801G + K875T + N892Y | | | | |
| A190Q + E229S + D458S + T631N + N672D + | 37 | 90 | 120 | 116 |
| G753E + S754E + S757D + A769D + L775A + | | | | |
| D801G + K875T + N892Y | | | | |
| Q109R + A159P + E229S + S635Q + T649K + | 37 | 90 | 120 | 116 |
| N672D + G753E + S754E + S757D + A769D + | | | | |
| L775A + D801G + K875T + N892Y | | | | |
| E229S + N440K + S582K + N672D + P752R + | 37 | 90 | 120 | 116 |
| G753E + S754E + A769D + L775A + D801G + | | | | |
| A843P + K875T + N892Y | | | | |
| A190Q + E229S + T631N + N672D + I703L + | 37 | 90 | 120 | 116 |
| P752K + G753E + A769D + L775A + D801G + | | | | |
| K875T + N892Y | | | | |
| T18D + E229S + S582K + N672D + G753E + | 37 | 90 | 120 | 117 |
| S754E + A769D + L775A + D801G + K875T + | | | | |
| N892Y | | | | |
| E229S + D458S + T631E + S635E + N672D + | 37 | 90 | 120 | 117 |
| G753E + S754E + A769D + L775A + D801G + | | | | |
| K875T + N892Y | | | | |
| E229S + A492L + S582K + S635E + T649K + | 37 | 90 | 120 | 118 |
| I656V + N672D + G753E + A769D + L775A + | | | | |
| D801G + K875T + N892Y | | | | |
| S100D + E229S + S635E + T649K + I656V + | 37 | 90 | 120 | 118 |
| N672D + P752K + G753E + S754E + A769D + | | | | |
| L775A + D801G + K875T + N892Y | | | | |
| E229S + K360G + D458S + S582K + N672D + | 37 | 90 | 120 | 118 |
| G753E + S754E + A769D + L775A + V800P + | | | | |
| D801G + K875T + N892Y + N1008D | | | | |
| E229S + A624E + S635E + T649K + I656V + | 37 | 90 | 120 | 118 |
| N672D + M728V + G753E + S754R + S757D + | | | | |
| A769D + L775A + D801G + K875T + N892Y | | | | |
| E229S + D458S + K567R + S635E + N672D + | 37 | 90 | 120 | 118 |
| I703L + G753E + S754E + A769D + L775A + | | | | |
| D801G + K875T + N892Y | | | | |
| Q109R + E229S + K360G + T631N + N672D + | 37 | 90 | 120 | 119 |
| P752R + G753E + S754E + S757D + A769D + | | | | |
| L775A + D801G + K875T + N892Y | | | | |
| E229S + K360G + D458S + K567R + S582K + | 37 | 90 | 120 | 119 |
| S635E + N672D + G753E + S754E + A769D + | | | | |
| L775A + D801G + K875T + N892Y | | | | |
| E229S + N440K + A492L + S582K + T631N + | 37 | 90 | 120 | 120 |
| N672D + G753E + S754E + A769D + L775A + | | | | |
| D801G + K875T + N892Y + N1008D | | | | |
| E229S + I234V + S582K + N672D + G753E + | 37 | 90 | 120 | 121 |
| S754E + S757D + A769D + L775A + D801G + | | | | |
| K875T + N892Y | | | | |
| E229S + N440K + A492L + S582K + A624E + | 37 | 90 | 120 | 121 |
| S635E + N672D + G753E + S754E + A769D + | | | | |
| L775A + D801G + K875T + N892Y | | | | |
| A159P + A190Q + E229S + I234V + S582K + | 37 | 90 | 120 | 122 |
| N672D + G753E + S754E + A769D + L775A + | | | | |
| D801G + K875T + N892Y | | | | |
| S100D + E229S + D458S + K567R + S635E + | 37 | 90 | 120 | 123 |
| N672D + G753E + S754E + A769D + L775A + | | | | |
| D801G + K875T + N892Y | | | | |
| A190Q + E229S + N440K + S582K + T631N + | 37 | 90 | 120 | 124 |
| N672D + G753E + S754E + A769D + L775A + | | | | |
| D801G + K875T + N892Y + N1008D | | | | |
| S100D + Q109R + E229S + R284G + T631N + | 37 | 90 | 120 | 125 |
| S635E + T649K + N672D + P752R + G753E + | | | | |
| S754E + A769D + L775A + D801G + K875T + | | | | |
| N892Y | | | | |
| E229S + V352I + S635E + T649K + I656V + | 37 | 90 | 120 | 125 |
| N672D + G753E + S754E + A769D + L775A + | | | | |
| V800P + D801G + K875T + N892Y | | | | |
| T18D + Q109R + E229S + T631N + N672D + | 37 | 90 | 120 | 126 |
| P752R + G753E + S754E + S757D + A769D + | | | | |
| L775A + D801G + K875T + N892Y | | | | |
| S100D + A190Q + E229S + I234V + S582K + | 37 | 90 | 120 | 129 |
| N672D + G753E + S754E + A769D + L775A + | | | | |
| D801G + K875T + N892Y | | | | |
| S100D + E229S + K360G + D458S + S582K + | 37 | 90 | 120 | 129 |
| N672D + G753E + S754E + A769D + L775A + | | | | |
| D777K + D801G + K875T + N892Y + N1008D | | | | |
| E229S + K360G + D458S + A492L + S582K + | 37 | 90 | 120 | 130 |
| N672D + G753E + S754E + A769D + L775A + | | | | |
| D801G + K875T + N892Y + N1008D | | | | |
| E229S + K360G + D458S + S582K + N672D + | 37 | 90 | 120 | 130 |
| G753E + S754E + A769D + L775A + D801G + | | | | |
| K875T + N892Y + N1008D | | | | |
| S100D + A190Q + E229S + K360G + D458S + | 37 | 90 | 120 | 132 |
| S582K + N672D + G753E + S754E + A769D + | | | | |
| L775A + D801G + K875T + N892Y + N1008D | | | | |
| E229S + A492L + S635E + T649K + I656V + | 37 | 90 | 120 | 134 |
| N672D + G753E + S757D + A769D + L775A + | | | | |
| D801G + K875T + N892Y | | | | |
| E229S + A624E + S635E + T649K + I656V + | 37 | 90 | 120 | 135 |
| N672D + G753E + S754R + S757D + A769D + | | | | |
| L775A + D777K + D801G + K875T + N892Y | | | | |
| A190Q + E229S + S635E + T649K + I656V + | 37 | 90 | 120 | 135 |
| N672D + P752K + G753E + A769D + L775A + | | | | |
| D801G + A843P + K875T + N892Y | | | | |
| T18D + E229S + A624E + S635E + T649K + | 37 | 90 | 120 | 136 |
| I656V + N672D + G753E + S754R + S757D + | | | | |
| A769D + L775A + D777K + D801G + K875T + | | | | |
| N892Y | | | | |
| E229S + S582K + S635E + N672D + P752R + | 37 | 90 | 120 | 136 |
| G753E + S754E + A769D + L775A + D801G + | | | | |
| K875T + N892Y + N1008D | | | | |
| A190Q + E229S + N440K + S582K + A624E + | 37 | 90 | 120 | 136 |
| S635E + N672D + G753E + S754E + A769D + | | | | |
| L775A + D801G + K875T + N892Y | | | | |
| E229S + S582K + S635E + T649K + I656V + | 37 | 90 | 120 | 137 |
| N672D + M728V + G753E + S754R + S757D + | | | | |
| A769D + L775A + D801G + K875T + N892Y | | | | |
| E229S + D458S + K567R + S582K + S635E + | 37 | 90 | 120 | 137 |
| N672D + I703L + G753E + S754E + A769D + | | | | |
| L775A + D801G + K875T + N892Y | | | | |
| E229S + I234V + S582K + N672D + G753E + | 37 | 90 | 120 | 137 |
| S754E + A769D + L775A + V800P + D801G + | | | | |
| K875T + N892Y | | | | |
| E229S + S635E + T649K + I656V + N672D + | 37 | 90 | 120 | 137 |
| P752K + G753E + S757D + A769D + L775A + | | | | |
| D801G + A843P + K875T + N892Y | | | | |
| Q109R + E229S + T631N + N672D + P752R + | 37 | 90 | 120 | 138 |
| G753E + S754E + S757D + A769D + L775A + | | | | |
| D801G + K875T + N892Y + K1016T | | | | |
| E229S + D458S + A624E + S635E + T649K + | 37 | 90 | 120 | 138 |
| I656V + N672D + G753E + S754R + S757D + | | | | |
| A769D + L775A + D777K + D801G + K875T + | | | | |
| N892Y | | | | |
| A190Q + E229S + K360G + D458S + S582K + | 37 | 90 | 120 | 141 |
| N672D + G753E + S754E + A769D + L775A + | | | | |
| D801G + K875T + N892Y + N1008D | | | | |
| S100D + E229S + N440K + S582K + T631N + | 37 | 90 | 120 | 142 |
| N672D + G753E + S754E + A769D + L775A + | | | | |
| D801G + K875T + N892Y + N1008D | | | | |
| S100D + E229S + K360G + D458S + S582K + | 37 | 90 | 120 | 144 |
| N672D + G753E + S754E + A769D + L775A + | | | | |
| D801G + A843P + K875T + N892Y + N1008D | | | | |
| E229S + I234V + A492L + N672D + G753E + | 37 | 90 | 120 | 145 |
| S754E + A769D + L775A + D777K + D801G + | | | | |
| K875T + N892Y | | | | |
| A190Q + E229S + K360G + D458S + S582K + | 37 | 90 | 120 | 146 |
| T664K + N672D + G753E + S754E + A769D + | | | | |
| L775A + D801G + K875T + N892Y + N1008D | | | | |
| A190Q + E229S + D458S + T631N + N672D + | 37 | 90 | 120 | 147 |
| G753E + S754E + A769D + L775A + D801G + | | | | |
| A843P + K875T + N892Y | | | | |
| E229S + N440K + S582K + S635E + N672D + | 37 | 90 | 120 | 147 |
| G753E + S754E + A769D + L775A + D801G + | | | | |
| A843P + K875T + N892Y + N1008D | | | | |
| E229S + A492L + S635E + T649K + I656V + | 37 | 90 | 120 | 147 |
| N672D + P752R + G753E + S754E + A769D + | | | | |
| L775A + D801G + K875T + N892Y + N1008D | | | | |
| T18D + E229S + S582K + N672D + G753E + | 37 | 90 | 120 | 149 |
| S754E + A769D + L77 5A + D801G + K875T + | | | | |
| N892Y + T902F | | | | |
| E229S + D458S + S582K + A624E + T631N + | 37 | 90 | 120 | 149 |
| N672D + M728V + G753E + S754E + S757D + | | | | |
| A769D + L775A + D801G + K875T + N892Y | | | | |
| E229S + D458S + T631E + N672D + G753E + | 37 | 90 | 120 | 151 |
| S754E + S757D + A769D + L775A + D801G + | | | | |
| K875T + N892Y | | | | |
| T18D + E229S + N440K + S582K + A624E + | 37 | 90 | 120 | 152 |
| S635E + N672D + G753E + S754E + S757D + | | | | |
| A769D + L775A + D801G + K875T + N892Y | | | | |
| E229S + N440K + S582K + A624E + N672D + | 37 | 90 | 120 | 152 |
| P752R + G753E + S754E + A769D + L775A + | | | | |
| D801G + K875T + N892Y + N1008D | | | | |
| E229S + N440K + S582K + A624E + T631N + | 37 | 90 | 120 | 153 |
| S635E + N672D + G753E + S754E + S757D + | | | | |
| A769D + L775A + D801G + K875T + N892Y | | | | |
| E229S + N440K + S582K + A624E + S635E + | 37 | 90 | 120 | 154 |
| N672D + G738L + G753E + S754E + S757D + | | | | |
| A769D + L775A + D801G + K875T + N892Y | | | | |
| E229S + I234V + A492L + S582K + N672D + | 37 | 90 | 120 | 157 |
| M728V + G753E + S754E + A769D + L775A + | | | | |
| D801G + K875T + N892Y | | | | |
| E229S + D458S + A492L + T631N + N672D + | 37 | 90 | 120 | 158 |
| G753E + S754E + S757D + A769D + L775A + | | | | |
| D801G + K875T + N892Y | | | | |
| A190Q + E229S + I234V + S582K + N672D + | 37 | 90 | 120 | 158 |
| G753E + S754E + S757D + A769D + L775A + | | | | |
| D801G + K875T + N892Y | | | | |
| E229S + N440K + S582K + N672D + P752R + | 37 | 90 | 120 | 158 |
| G753E + S754E + S757D + A769D + L775A + | | | | |
| D801G + K875T + N892Y + N1008D | | | | |
| E229S + S635E + T649K + I656V + N672D + | 37 | 90 | 120 | 159 |
| G753E + S754R + S757D + A769D + L775A + | | | | |
| D801G + A843P + K875T + N892Y | | | | |
| A190Q + E229S + N440K + S582K + A624E + | 37 | 90 | 120 | 162 |
| S635E + N672D + G753E + S754E + S757D + | | | | |
| A769D + L775A + D801G + K875T + N892Y | | | | |
| S100D + E229S + D458S + K567R + S582K + | 37 | 90 | 120 | 163 |
| S635E + N672D + G753E + S754E + A769D + | | | | |
| L775A + D801G + K875T + N892Y | | | | |
| E229S + S635E + N672D + P752R + G753E + | 37 | 90 | 120 | 169 |
| S754E + A769D + L775A + D777K + D801G + | | | | |
| K875T + N892Y | | | | |
| S100D + A190Q + E229S + K360G + D458S + | 37 | 90 | 120 | 170 |
| S582K + N672D + G753E + S754E + A769D + | | | | |
| L775A + D801G + K875T + N892Y + N1008D | | | | |
| S100D + E229S + K360G + D458S + S582K + | 37 | 90 | 120 | 171 |
| N672D + G753E + S754E + A769D + L775A + | | | | |
| D801G + K875T + N892Y + N1008D | | | | |
| E229S + D458S + S582K + T631N + S635E + | 37 | 90 | 120 | 172 |
| N672D + M728V + G753E + S754E + S757D + | | | | |
| A769D + L775A + D801G + K875T + N892Y | | | | |
| S100D + E229S + K360G + D458S + S582K + | 37 | 90 | 120 | 174 |
| N672D + G753E + S754E + S757D + A769D + | | | | |
| L775A + D801G + A843P + K875T + N892Y + | | | | |
| N1008D | | | | |
| E229S + I234V + A492L + S582K + N672D + | 37 | 90 | 120 | 174 |
| G753E + S754E + A769D + L775A + D801G + | | | | |
| K875T + N892Y | | | | |
| E229S + D458S + S582K + T631E + N672D + | 37 | 90 | 120 | 175 |
| G753E + S754E + A769D + L775A + D801G + | | | | |
| K875T + N892Y | | | | |
| E229S + I234V + A492L + N672D + G753E + | 37 | 90 | 120 | 176 |
| S754E + A769D + L775A + D777R + D801G + | | | | |
| K875T + N892Y | | | | |
| S100D + E229S + K360G + D458S + S582K + | 37 | 90 | 120 | 179 |
| N672D + G753E + S754E + S757D + A769D + | | | | |
| L775A + D801G + A843P + K875T + N892Y + | | | | |
| N1008D | | | | |
| E229S + A624E + S635E + T649K + I656V + | 37 | 90 | 120 | 185 |
| N672D + G753E + S754R + S757D + A769D + | | | | |
| L775A + D777K + D801G + K875T + N892Y | | | | |
| S100D + E229S + K360G + D458S + S582K + | 37 | 90 | 120 | 189 |
| N672D + G753E + S754E + S757D + A769D + | | | | |
| L775A + D801G + A843P + K875T + N892Y + | | | | |
| T915A + N1008D | | | | |
| T18D + E229S + D458S + T631N + N672D + | 37 | 90 | 120 | 191 |
| M728V + G753E + S754E + S757D + A769D + | | | | |
| L775A + D801G + K875T + N892Y | | | | |
| S100D + E229S + N440K + S582K + N672D + | 37 | 90 | 120 | 192 |
| P752R + G753E + S754E + A769D + L775A + | | | | |
| D801G + K875T + N892Y + N1008D | | | | |
| T18D + E229S + S582K + N672D + G753E + | 37 | 90 | 120 | 202 |
| S754E + P764K + A769D + L775A + D801G + | | | | |
| K875T + N892Y | | | | |
| E229S + D458S + T631E + N672D + G753E + | 37 | 90 | 120 | 203 |
| S754E + A769D + L775A + D777K + D801G + | | | | |
| K875T + N892Y | | | | |
| E229S + S582K + S635E + T649K + I656V + | 37 | 90 | 120 | 209 |
| N672D + P752K + G753E + S757D + A769D + | | | | |
| L775A + D801G + A843P + K875T + N892Y | | | | |
| E229S + A624E + S635E + T649K + I656V + | 37 | 90 | 120 | 210 |
| N672D + G738L + G753E + S754R + S757D + | | | | |
| A769D + L775A + D777K + D801G + K875T + | | | | |
| N892Y | | | | |
| A159P + E229S + D458S + T631N + N672D + | 37 | 90 | 120 | 223 |
| M728V + G753E + S754E + S757D + A769D + | | | | |
| L775A + D801G + K875T + N892Y | | | | |
| A190Q + E229S + S582K + N672D + G753E + | 37 | 90 | 120 | 226 |
| S754E + A769D + L775A + D801G + K875T + | | | | |
| N892Y | | | | |
| A190Q + E229S + K360G + D458S + S582K + | 37 | 90 | 120 | 234 |
| N672D + G753E + S754E + S757D + A769D + | | | | |
| L775A + D801G + K875T + N892Y + N1008D | | | | |

### Example 10

### Mini-TOM Wash of Xanthan Lyase and Endoglucanase Wildtypes and Variants

To assess improved detergent stability of xanthan lyase (XL) variants and endoglucanase (EG) variants over their respective wildtypes (XL and EG wildtypes), the XL and EG wildtypes as well as combinations of XL and EG variants were incubated in heavy duty liquid detergent (Persil Universal gel, PUG) for 13 days at 30° C. after which they were subjected to a wash test in a mini-TOM (mini-terg-O-tometer) wash setup. The wash performance of the enzyme combinations stored 13 days in liquid detergent were compared to an identical mini-TOM wash trial in which the detergent and enzymes had not been stored prior to the wash (i.e. fresh wash trial), but were added directly to the wash just before starting the wash.

In brief, mini-TOM wash is a down-scaled version of TOM wash (terg-O-tometer wash, see WO2016/203064 for reference to the method) in which each beaker is 300 mL in size (instead of 2 L) and is filled with 100 mL wash liqour (instead of 1 L). All other parameters (e.g. agitation) are identical to standard TOM wash.

Storage of enzymes in detergent: XL enzymes were diluted to 227 ppm and EG enzymes were diluted to 454 ppm in MQ water. Glass vials with lids were prepared with 0.90 g of PUG detergent. 50 μL of diluted XL enzyme and 50 μL of diluted EG enzyme (wildtypes or variants) were added to the same glass vial containing 0.90 g of PUG detergent. The glass vials were stored 13 days in a heating cabinet at 30° C.

Dosing in wash (stored samples): For each mini-TOM beaker 0.44 g stored PUG detergent with enzymes were diluted in 100 mL 16° dH water hardness solution (Ca:Mg 5:1, HCO₃×1.5) to give a final concentration of 0.05 ppm XL and 0.1 ppm EG.

Dosing in wash (fresh samples): For each mini-TOM beaker 0.44 g PUG detergent were diluted in 100 mL 16 ° dH water hardness solution (Ca:Mg 5:1, HCO₃×1.5) and added diluted XL and EG enzymes (wildtypes or variants) directly to each mini-TOM beaker to give a final concentration of 0.05 ppm XL and 0.1 ppm EG. Reference samples (i.e. detergent without XL and EG enzymes) were included in the wash trials to assess the detergent cleaning level without XL and EG enzyme (reference).

Mini-TOM wash (stored or fresh samples): Each mini-TOM beaker containing 100 mL of the PUG detergent wash liquor (4.4 g PUG/L wash liqour) with enzymes (stored or fresh) and 2 pieces of each of the commercially available stains C-S-05S (Mayonnaise with carbon black on Cotton/soot—see Table 1 for results), C-S-17 (Fluid Make up on Cotton—see Table 3 for results) and C-S-44 (Chocolate drink pure on Cotton—see Table 2 for results) (Center for Testmaterials b.v., Netherlands) cut into 2 cm0 circular stains was washed at 40° C. for 30 min at 120 rpm, then rinsed in cold tap water for 30 seconds, then placed on a sheet of filter paper to dry overnight at room temperature and then transferred to a measuring plate.

Calculation of residual wash performance: The intensity of the stains was measured using a Digi-eye (Tiff files), and Coloreye software was used to acquire the intensity data. Using the Intensity data of the stored and fresh enzyme samples and the reference samples, Delta performance (Delta Intensity, DI) and Residual performance (RP) for each stain were calculated. Briefly, for each enzyme combination DI was calculated by subtracting the reference intensity from each measured intensity. Furthermore, Residual performance was calculated for each combination of enzymes by the following formula: RP=DI_{stored}/DI_{Fresh}* 100%.

TABLE 34
Residual wash performance (RP) of XL (xanthan lyase) and EG (endoglucanase)
enzyme combinations (wildtypes or variants) after storage 13 days in PUG
detergent at 30° C. Tested on Mayo with soot (CS-05S) swatches.

| Enzyme mutations relative to SEQ ID NO: 2 | | Mayo with soot, CS-05S | | |
|---|---|---|---|---|
| (EG) and relative to SEQ ID NO: 6 (XL) | | Delta Intensity | | |
| Endoglucanase (EG) | Xanthan lyase (XL) | Fresh | Stored | RP |
| none, wildtype | none, wildtype | 17 | 2 | 15% |
| A559N + Y579W + T697G | E229N + N672D + P752K + G753E + | 16 | 5 | 30% |
| | A769D + L775A + D801G + K875T + | | | |
| | N892Y | | | |
| A559N + Y579W + T697G | E229S + N672D + G753E + S754E + | 16 | 7 | 43% |
| | A769D + L775A + D801G + K875T + | | | |
| | N892Y | | | |
| A559N + Y579W + T697G | E229S + N672D + P752R + G753E + | 19 | 8 | 44% |
| | S754E + A769D + L775A + D801G + | | | |
| | K875T + N892Y | | | |
| A559N + Y579W + T697G | A190Q + E229S + I234V + A624E + | 18 | 8 | 43% |
| | N672D + G753E + S754E + A769D + | | | |
| | L775A + D801G + K875T | | | |
| A559N + Y579W + T697G | A190Q + E229S + T631N + N672D + | 17 | 8 | 46% |
| | I703L + P752K + G753E + A769D + | | | |
| | L775A + D801G + K875T | | | |
| A559N + Y579W + T697G | A190Q + E229S + I234V + S582K + | 19 | 12 | 65% |
| | N672D + G753E + S754E + S757D + | | | |
| | A769D + L775A + D801G + K875T + | | | |
| | N892Y | | | |
| A559N + Y579W + T697G | E229S + N440K + S582K + A624E + | 14 | 11 | 78% |
| | S635E + N672D + G738L + G753E + | | | |
| | S754E + S757D + A769D + L775A + | | | |
| | D801G + K875T + N892Y | | | |
| K512P + A559N + Y579W + T697G | E229S + N672D + G753E + S754E + | 15 | 7 | 47% |
| | A769D + L775A + D801G + K875T + | | | |
| | N892Y | | | |
| K512P + A559N + Y579W + T697G | E229S + N672D + P752R + G753E + | 17 | 9 | 54% |
| | S754E + A769D + L775A + D801G + | | | |
| | K875T + N892Y | | | |
| K512P + A559N + Y579W + T697G | A190Q + E229S + I234V + A624E + | 16 | 8 | 48% |
| | N672D + G753E + S754E + A769D + | | | |
| | L775A + D801G + K875T | | | |
| K512P + A559N + Y579W + T697G | A190Q + E229S + T631N + N672D + | 17 | 9 | 54% |
| | I703L + P752K + G753E + A769D + | | | |
| | L775A + D801G + K875T | | | |
| K512P + A559N + Y579W + T697G | A190Q + E229S + I234V + S582K + | 17 | 11 | 66% |
| | N672D + G753E + S754E + S757D + | | | |
| | A769D + L775A + D801G + K875T + | | | |
| | N892Y | | | |
| K512P + A559N + Y579W + T697G | E229S + N440K + S582K + A624E + | 16 | 13 | 84% |
| | S635E + N672D + G738L + G753E + | | | |
| | S754E + S757D + A769D + L775A + | | | |
| | D801G + K875T + N892Y | | | |
| N18G + A71E + A186P + | E229S + N672D + G753E + S754E + | 17 | 6 | 34% |
| E408D + Y579W + I602T + | A769D + L775A + D801G + K875T + | | | |
| A651P + A688G + V756Y | N892Y | | | |
| N18G + A71E + A186P + | E229S + N672D + P752R + G753E + | 17 | 7 | 44% |
| E408D + Y579W + I602T + | S754E + A769D + L775A + D801G + | | | |
| A651P + A688G + V756Y | K875T + N892Y | | | |
| N18G + A71E + A186P + | A190Q + E229S + I234V + A624E + | 16 | 6 | 40% |
| E408D + Y579W + I602T + | N672D + G753E + S754E + A769D + | | | |
| A651P + A688G + V756Y | L775A + D801G + K875T | | | |
| N18G + A71E + A186P + | A190Q + E229S + T631N + N672D + | 16 | 7 | 46% |
| E408D + Y579W + 1602T + | I703L + P752K + G753E + A769D + | | | |
| A651P + A688G + V756Y | L775A + D801G + K875T | | | |
| N18G + A71E + A186P + | A190Q + E229S + I234V + S582K + | 15 | 10 | 69% |
| E408D + Y579W + I602T + | N672D + G753E + S754E + S757D + | | | |
| A651P + A688G + V756Y | A769D + L775A + D801G + K875T + | | | |
| | N892Y | | | |
| N18G + A71E + A186P + | E229S + N440K + S582K + A624E + | 13 | 8 | 61% |
| E408D + Y579W + I602T + | S635E + N672D + G738L + G753E + | | | |
| A651P + A688G + V756Y | S754E + S757D + A769D + L775A + | | | |
| | D801G + K875T + N892Y | | | |
| N18G + N189K + E408D + | E229N + N672D + P752K + G753E + | 17 | 4 | 24% |
| A559N + Y579W + A688G + | A769D + L775A + D801G + K875T + | | | |
| T697G + V756Y + K921R + | N892Y | | | |
| Y934G | | | | |
| N18G + N189K + E408D + | E229S + N672D + G753E + S754E + | 15 | 5 | 31% |
| A559N + Y579W + A688G + | A769D + L775A + D801G + K875T + | | | |
| T697G + V756Y + K921R + | N892Y | | | |
| Y934G | | | | |
| N18G + N189K + E408D + | E229S + N672D + P752R + G753E + | 19 | 8 | 41% |
| A559N + Y579W + A688G + | S754E + A769D + L775A + D801G + | | | |
| T697G + V756Y + K921R + | K875T + N892Y | | | |
| Y934G | | | | |
| N18G + N189K + E408D + | A190Q + E229S + I234V + A624E + | 19 | 6 | 31% |
| A559N + Y579W + A688G + | N672D + G753E + S754E + A769D + | | | |
| T697G + V756Y + K921R + | L775A + D801G + K875T | | | |
| Y934G | | | | |
| N18G + N189K + E408D + | A190Q + E229S + T631N + N672D + | 17 | 8 | 44% |
| A559N + Y579W + A688G + | I703L + P752K + G753E + A769D + | | | |
| T697G + V756Y + K921R + | L775A + D801G + K875T | | | |
| Y934G | | | | |
| N18G + N189K + E408D + | A190Q + E229S + I234V + S582K + | 19 | 12 | 64% |
| A559N + Y579W + A688G + | N672D + G753E + S754E + S757D + | | | |
| T697G + V756Y + K921R + | A769D + L775A + D801G + K875T + | | | |
| Y934G | N892Y | | | |
| N18G + N189K + E408D + | E229S + N440K + S582K + A624E + | 17 | 14 | 84% |
| A559N + Y579W + A688G + | S635E + N672D + G738L + G753E + | | | |
| T697G + V756Y + K921R + | S754E + S757D + A769D + L775A + | | | |
| Y934G | D801G + K875T + N892Y | | | |
| S313D + E408D | E229N + N672D + P752K + G753E + | 17 | 5 | 31% |
| | A769D + L775A + D801G + K875T + | | | |
| | N892Y | | | |
| S313D + E408D | E229S + N672D + G753E + S754E + | 17 | 6 | 35% |
| | A769D + L775A + D801G + K875T + | | | |
| | N892Y | | | |
| S313D + E408D | E229S + N672D + P752R + G753E + | 19 | 10 | 51% |
| | S754E + A769D + L775A + D801G + | | | |
| | K875T + N892Y | | | |
| S313D + E408D | A190Q + E229S + I234V + A624E + | 18 | 8 | 48% |
| | N672D + G753E + S754E + A769D + | | | |
| | L775A + D801G + K875T | | | |
| S313D + E408D | A190Q + E229S + T631N + N672D + | 17 | 7 | 44% |
| | I703L + P752K + G753E + A769D + | | | |
| | L775A + D801G + K875T | | | |
| S313D + E408D | A190Q + E229S + I234V + S582K + | 18 | 13 | 74% |
| | N672D + G753E + S754E + S757D + | | | |
| | A769D + L775A + D801G + K875T + | | | |
| | N892Y | | | |
| S313D + E408D | E229S + N440K + S582K + A624E + | 14 | 10 | 69% |
| | S635E + N672D + G738L + G753E + | | | |
| | S754E + S757D + A769D + L775A + | | | |
| | D801G + K875T + N892Y | | | |
| R880K + N905D + K921R + Y934G | E229N + N672D + P752K + G753E + | 18 | 5 | 29% |
| | A769D + L775A + D801G + K875T + | | | |
| | N892Y | | | |
| R880K + N905D + K921R + Y934G | E229S + N672D + G753E + S754E + | 18 | 7 | 41% |
| | A769D + L775A + D801G + K875T + | | | |
| | N892Y | | | |
| R880K + N905D + K921R + Y934G | E229S + N672D + P752R + G753E + | 20 | 8 | 37% |
| | S754E + A769D + L775A + D801G + | | | |
| | K875T + N892Y | | | |
| R880K + N905D + K921R + Y934G | A190Q + E229S + I234V + A624E + | 17 | 8 | 47% |
| | N672D + G753E + S754E + A769D + | | | |
| | L775A + D801G + K875T | | | |
| R880K + N905D + K921R + Y934G | A190Q + E229S + T631N + N672D + | 21 | 9 | 44% |
| | I703L + P752K + G753E + A769D + | | | |
| | L775A + D801G + K875T | | | |
| R880K + N905D + K921R + Y934G | A190Q + E229S + I234V + S582K + | 20 | 13 | 67% |
| | N672D + G753E + S754E + S757D + | | | |
| | A769D + L775A + D801G + K875T + | | | |
| | N892Y | | | |
| R880K + N905D + K921R + Y934G | E229S + N440K + S582K + A624E + | 19 | 14 | 74% |
| | S635E + N672D + G738L + G753E + | | | |
| | S754E + S757D + A769D + L775A + | | | |
| | D801G + K875T + N892Y | | | |
| I302D + S313D + E408D + | E229N + N672D + P752K + G753E + | 17 | 4 | 24% |
| Y579W + I602T + A651P + | A769D + L775A + D801G + K875T + | | | |
| T697G + R880K + K921R + | N892Y | | | |
| Y934G | | | | |
| I302D + S313D + E408D + | E229S + N672D + G753E + S754E + | 17 | 6 | 36% |
| Y579W + I602T + A651P + | A769D + L775A + D801G + K875T + | | | |
| T697G + R880K + K921R + | N892Y | | | |
| Y934G | | | | |
| I302D + S313D + E408D + | E229S + N672D + P752R + G753E + | 18 | 8 | 45% |
| Y579W + I602T + A651P + | S754E + A769D + L775A + D801G + | | | |
| T697G + R880K + K921R + | K875T + N892Y | | | |
| Y934G | | | | |
| I302D + S313D + E408D + | A190Q + E229S + I234V + A624E + | 17 | 7 | 40% |
| Y579W + I602T + A651P + | N672D + G753E + S754E + A769D + | | | |
| T697G + R880K + K921R + | L775A + D801G + K875T | | | |
| Y934G | | | | |
| I302D + S313D + E408D + | A190Q + E229S + T631N + N672D + | 18 | 6 | 33% |
| Y579W + I602T + A651P + | I703L + P752K + G753E + A769D + | | | |
| T697G + R880K + K921R + | L775A + D801G + K875T | | | |
| Y934G | | | | |
| I302D + S313D + E408D + | A190Q + E229S + I234V + S582K + | 18 | 11 | 63% |
| Y579W + I602T + A651P + | N672D + G753E + S754E + S757D + | | | |
| T697G + R880K + K921R + | A769D + L775A + D801G + K875T + | | | |
| Y934G | N892Y | | | |
| I302D + S313D + E408D + | E229S + N440K + S582K + A624E + | 14 | 11 | 79% |
| Y579W + I602T + A651P + | S635E + N672D + G738L + G753E + | | | |
| T697G + R880K + K921R + | S754E + S757D + A769D + L775A + | | | |
| Y934G | D801G + K875T + N892Y | | | |
| N216Q + S313D + E408D + | E229N + N672D + P752K + G753E + | 16 | 10 | 61% |
| D476R + Y579W + I602T + | A769D + L775A + D801G + K875T + | | | |
| F638N + A651P + T697G + | N892Y | | | |
| W719R + R880K + T887K + | | | | |
| K921R + Y934G | | | | |
| N216Q + S313D + E408D + | E229S + N672D + G753E + S754E + | 17 | 11 | 63% |
| D476R + Y579W + I602T + | A769D + L775A + D801G + K875T + | | | |
| F638N + A651P + T697G + | N892Y | | | |
| W719R + R880K + T887K + | | | | |
| K921R + Y934G | | | | |
| N216Q + S313D + E408D + | E229S + N672D + P752R + G753E + | 19 | 14 | 74% |
| D476R + Y579W + I602T + | S754E + A769D + L775A + D801G + | | | |
| F638N + A651P + T697G + | K875T + N892Y | | | |
| W719R + R880K + T887K + | | | | |
| K921R + Y934G | | | | |
| N216Q + S313D + E408D + | A190Q + E229S + I234V + A624E + | 17 | 12 | 73% |
| D476R + Y579W + I602T + | N672D + G753E + S754E + A769D + | | | |
| F638N + A651P + T697G + | L775A + D801G + K875T | | | |
| W719R + R880K + T887K + | | | | |
| K921R + Y934G | | | | |
| N216Q + S313D + E408D + | A190Q + E229S + T631N + N672D + | 19 | 14 | 75% |
| D476R + Y579W + I602T + | I703L + P752K + G753E + A769D + | | | |
| F638N + A651P + T697G + | L775A + D801G + K875T | | | |
| W719R + R880K + T887K + | | | | |
| K921R + Y934G | | | | |
| N216Q + S313D + E408D + | A190Q + E229S + I234V + S582K + | 20 | 16 | 80% |
| D476R + Y579W + I602T + | N672D + G753E + S754E + S757D + | | | |
| F638N + A651P + T697G + | A769D + L775A + D801G + K875T + | | | |
| W719R + R880K + T887K + | N892Y | | | |
| K921R + Y934G | | | | |
| N216Q + S313D + E408D + | E229S + N440K + S582K + A624E + | 19 | 18 | 97% |
| D476R + Y579W + I602T + | S635E + N672D + G738L + G753E + | | | |
| F638N + A651P + T697G + | S754E + S757D + A769D + L775A + | | | |
| W719R + R880K + T887K + | D801G + K875T + N892Y | | | |
| K921R + Y934G | | | | |

TABLE 35
Residual wash performance (RP) of XL and EG enzyme combinations (wildtypes or variants) after
storage 13 days in PUG detergent at 30° C. Tested on Chocolate drink (CS-44) swatches.

| Enzyme mutations relative to SEQ ID NO: 2 | | Chocolate drink, CS-44 | | |
|---|---|---|---|---|
| (EG) and relative to SEQ ID NO: 6 (XL) | | Delta Intensity | | |
| Endoglucanase (EG) | Xanthan lyase (XL) | Fresh | Stored | RP |
| none, wildtype | none, wildtype | 14 | 3 | 23% |
| A559N + Y579W + T697G | E229N + N672D + P752K + G753E + | 13 | 4 | 30% |
| | A769D + L775A + D801G + K875T + | | | |
| | N892Y | | | |
| A559N + Y579W + T697G | E229S + N672D + G753E + S754E + | 13 | 5 | 39% |
| | A769D + L775A + D801G + K875T + | | | |
| | N892Y | | | |
| A559N + Y579W + T697G | E229S + N672D + P752R + G753E + | 16 | 6 | 41% |
| | S754E + A769D + L775A + D801G + | | | |
| | K875T + N892Y | | | |
| A559N + Y579W + T697G | A190Q + E229S + I234V + A624E + | 13 | 7 | 49% |
| | N672D + G753E + S754E + A769D + | | | |
| | L775A + D801G + K875T+ | | | |
| A559N + Y579W + T697G | A190Q + E229S + T631N + N672D + | 15 | 6 | 40% |
| | I703L + P752K + G753E + A769D + | | | |
| | L775A + D801G + K875T+ | | | |
| A559N + Y579W + T697G | A190Q + E229S + I234V + S582K + | 12 | 9 | 75% |
| | N672D + G753E + S754E + S757D + | | | |
| | A769D + L775A + D801G + K875T + | | | |
| | N892Y | | | |
| A559N + Y579W + T697G | E229S + N440K + S582K + A624E + | 10 | 5 | 56% |
| | S635E + N672D + G738L + G753E + | | | |
| | S754E + S757D + A769D + L775A + | | | |
| | D801G + K875T + N892Y | | | |
| K512P + A559N + Y579W + T697G | E229S + N672D + G753E + S754E + | 11 | 4 | 39% |
| | A769D + L775A + D801G + K875T + | | | |
| | N892Y | | | |
| K512P + A559N + Y579W + T697G | E229S + N672D + P752R + G753E + | 12 | 6 | 52% |
| | S754E + A769D + L775A + D801G + | | | |
| | K875T + N892Y | | | |
| K512P + A559N + Y579W + T697G | A190Q + E229S + I234V + A624E + | 12 | 5 | 44% |
| | N672D + G753E + S754E + A769D + | | | |
| | L775A + D801G + K875T+ | | | |
| K512P + A559N + Y579W + T697G | A190Q + E229S + T631N + N672D + | 13 | 8 | 63% |
| | I703L + P752K + G753E + A769D + | | | |
| | L775A + D801G + K875T+ | | | |
| K512P + A559N + Y579W + T697G | A190Q + E229S + I234V + S582K + | 14 | 12 | 84% |
| | N672D + G753E + S754E + S757D + | | | |
| | A769D + L775A + D801G + K875T + | | | |
| | N892Y | | | |
| K512P + A559N + Y579W + T697G | E229S + N440K + S582K + A624E + | 11 | 9 | 77% |
| | S635E + N672D + G738L + G753E + | | | |
| | S754E + S757D + A769D + L775A + | | | |
| | D801G + K875T + N892Y | | | |
| N18G + A71E + A186P + E408D + | E229S + N672D + G753E + S754E + | 15 | 6 | 40% |
| Y579W + I602T + A651P + A688G + | A769D + L775A + D801G + K875T + | | | |
| V756Y | N892Y | | | |
| N18G + A71E + A186P + E408D + | A190Q + E229S + I234V + A624E + | 16 | 6 | 37% |
| Y579W + I602T + A651P + A688G + | N672D + G753E + S754E + A769D + | | | |
| V756Y | L775A + D801G + K875T+ | | | |
| N18G + A71E + A186P + E408D + | A190Q + E229S + T631N + N672D + | 16 | 5 | 29% |
| Y579W + I602T + A651P + A688G + | I703L + P752K + G753E + A769D + | | | |
| V756Y | L775A + D801G + K875T+ | | | |
| N18G + A71E + A186P + E408D + | A190Q + E229S + I234V + S582K + | 13 | 7 | 50% |
| Y579W + I602T + A651P + A688G + | N672D + G753E + S754E + S757D + | | | |
| V756Y | A769D + L775A + D801G + K875T + | | | |
| | N892Y | | | |
| N18G + A71E + A186P + E408D + | E229S + N440K + S582K + A624E + | 8 | 6 | 81% |
| Y579W + I602T + A651P + A688G + | S635E + N672D + G738L + G753E + | | | |
| V756Y | S754E + S757D + A769D + L775A + | | | |
| | D801G + K875T + N892Y | | | |
| N18G + N189K + E408D + A559N + | E229S + N672D + G753E + S754E + | 14 | 4 | 26% |
| Y579W + A688G + T697G + V756Y + | A769D + L775A + D801G + K875T + | | | |
| K921R + Y934G | N892Y | | | |
| N18G + N189K + E408D + A559N + | E229S + N672D + P752R + G753E + | 13 | 3 | 25% |
| Y579W + A688G + T697G + V756Y + | S754E + A769D + L775A + D801G + | | | |
| K921R + Y934G | K875T + N892Y | | | |
| N18G + N189K + E408D + A559N + | A190Q + E229S + T631N + N672D + | 16 | 7 | 46% |
| Y579W + A688G + T697G + V756Y + | I703L + P752K + G753E + A769D + | | | |
| K921R + Y934G | L775A + D801G + K875T+ | | | |
| N18G + N189K + E408D + A559N + | A190Q + E229S + I234V + S582K + | 15 | 8 | 55% |
| Y579W + A688G + T697G + V756Y + | N672D + G753E + S754E + S757D + | | | |
| K921R + Y934G | A769D + L775A + D801G + K875T + | | | |
| | N892Y | | | |
| N18G + N189K + E408D + A559N + | E229S + N440K + S582K + A624E + | 13 | 6 | 51% |
| Y579W + A688G + T697G + V756Y + | S635E + N672D + G738L + G753E + | | | |
| K921R + Y934G | S754E + S757D + A769D + L775A + | | | |
| | D801G + K875T + N892Y | | | |
| S313D + E408D | E229N + N672D + P752K + G753E + | 14 | 3 | 24% |
| | A769D + L775A + D801G + K875T + | | | |
| | N892Y | | | |
| S313D + E408D | E229S + N672D + G753E + S754E + | 15 | 4 | 28% |
| | A769D + L775A + D801G + K875T + | | | |
| | N892Y | | | |
| S313D + E408D | A190Q + E229S + I234V + A624E + | 15 | 3 | 23% |
| | N672D + G753E + S754E + A769D + | | | |
| | L775A + D801G + K875T+ | | | |
| S313D + E408D | A190Q + E229S + T631N + N672D + | 14 | 6 | 40% |
| | I703L + P752K + G753E + A769D + | | | |
| | L775A + D801G + K875T+ | | | |
| S313D + E408D | A190Q + E229S + I234V + S582K + | 13 | 8 | 59% |
| | N672D + G753E + S754E + S757D + | | | |
| | A769D + L775A + D801G + K875T + | | | |
| | N892Y | | | |
| S313D + E408D | E229S + N440K + S582K + A624E + | 10 | 8 | 79% |
| | S635E + N672D + G738L + G753E + | | | |
| | S754E + S757D + A769D + L775A + | | | |
| | D801G + K875T + N892Y | | | |
| R880K + N905D + K921R + Y934G | E229N + N672D + P752K + G753E + | 13 | 4 | 33% |
| | A769D + L775A + D801G + K875T + | | | |
| | N892Y | | | |
| R880K + N905D + K921R + Y934G | E229S + N672D + P752R + G753E + | 16 | 6 | 36% |
| | S754E + A769D + L775A + D801G + | | | |
| | K875T + N892Y | | | |
| R880K + N905D + K921R + Y934G | A190Q + E229S + I234V + A624E + | 15 | 6 | 36% |
| | N672D + G753E + S754E + A769D + | | | |
| | L775A + D801G + K875T+ | | | |
| R880K + N905D + K921R + Y934G | A190Q + E229S + T631N + N672D + | 16 | 5 | 31% |
| | I703L + P752K + G753E + A769D + | | | |
| | L775A + D801G + K875T+ | | | |
| R880K + N905D + K921R + Y934G | A190Q + E229S + I234V + S582K + | 14 | 12 | 82% |
| | N672D + G753E + S754E + S757D + | | | |
| | A769D + L775A + D801G + K875T + | | | |
| | N892Y | | | |
| R880K + N905D + K921R + Y934G | E229S + N440K + S582K + A624E + | 15 | 9 | 64% |
| | S635E + N672D + G738L + G753E + | | | |
| | S754E + S757D + A769D + L775A + | | | |
| | D801G + K875T + N892Y | | | |
| I302D + S313D + E408D + Y579W + | E229N + N672D + P752K + G753E + | 16 | 6 | 38% |
| I602T + A651P + T697G + R880K + | A769D + L775A + D801G + K875T + | | | |
| K921R + Y934G | N892Y | | | |
| I302D + S313D + E408D + Y579W + | E229S + N672D + G753E + S754E + | 18 | 4 | 23% |
| I602T + A651P + T697G + R880K + | A769D + L775A + D801G + K875T + | | | |
| K921R + Y934G | N892Y | | | |
| I302D + S313D + E408D + Y579W + | E229S + N672D + P752R + G753E + | 16 | 6 | 40% |
| I602T + A651P + T697G + R880K + | S754E + A769D + L775A + D801G + | | | |
| K921R + Y934G | K875T + N892Y | | | |
| I302D + S313D + E408D + Y579W + | A190Q + E229S + I234V + A624E + | 17 | 5 | 29% |
| I602T + A651P + T697G + R880K + | N672D + G753E + S754E + A769D + | | | |
| K921R + Y934G | L775A + D801G + K875T+ | | | |
| I302D + S313D + E408D + Y579W + | A190Q + E229S + T631N + N672D + | 18 | 5 | 27% |
| I602T + A651P + T697G + R880K + | I703L + P752K + G753E + A769D + | | | |
| K921R + Y934G | L775A + D801G + K875T+ | | | |
| I302D + S313D + E408D + Y579W + | A190Q + E229S + I234V + S582K + | 17 | 8 | 45% |
| I602T + A651P + T697G + R880K + | N672D + G753E + S754E + S757D + | | | |
| K921R + Y934G | A769D + L775A + D801G + K875T + | | | |
| | N892Y | | | |
| I302D + S313D + E408D + Y579W + | E229S + N440K + S582K + A624E + | 12 | 9 | 70% |
| I602T + A651P + T697G + R880K + | S635E + N672D + G738L + G753E + | | | |
| K921R + Y934G | S754E + S757D + A769D + L775A + | | | |
| | D801G + K875T + N892Y | | | |
| N216Q + S313D + E408D + D476R + | E229N + N672D + P752K + G753E + | 14 | 9 | 65% |
| Y579W + I602T + F638N + A651P + | A769D + L775A + D801G + K875T + | | | |
| T697G + W719R + R880K + T887K + | N892Y | | | |
| K921R + Y934G | | | | |
| N216Q + S313D + E408D + D476R + | E229S + N672D + G753E + S754E + | 14 | 10 | 66% |
| Y579W + I602T + F638N + A651P + | A769D + L775A + D801G + K875T + | | | |
| T697G + W719R + R880K + T887K + | N892Y | | | |
| K921R + Y934G | | | | |
| N216Q + S313D + E408D + D476R + | E229S + N672D + P752R + G753E + | 16 | 14 | 84% |
| Y579W + I602T + F638N + A651P + | S754E + A769D + L775A + D801G + | | | |
| T697G + W719R + R880K + T887K + | K875T + N892Y | | | |
| K921R + Y934G | | | | |
| N216Q + S313D + E408D + D476R + | A190Q + E229S + I234V + A624E + | 16 | 14 | 90% |
| Y579W + I602T + F638N + A651P + | N672D + G753E + S754E + A769D + | | | |
| T697G + W719R + R880K + T887K + | L775A + D801G + K875T+ | | | |
| K921R + Y934G | | | | |
| N216Q + S313D + E408D + D476R + | A190Q + E229S + T631N + N672D + | 18 | 13 | 74% |
| Y579W + I602T + F638N + A651P + | I703L + P752K + G753E + A769D + | | | |
| T697G + W719R + R880K + T887K + | L775A + D801G + K875T+ | | | |
| K921R + Y934G | | | | |
| N216Q + S313D + E408D + D476R + | A190Q + E229S + I234V + S582K + | 19 | 13 | 69% |
| Y579W + I602T + F638N + A651P + | N672D + G753E + S754E + S757D + | | | |
| T697G + W719R + R880K + T887K + | A769D + L775A + D801G + K875T + | | | |
| K921R + Y934G | N892Y | | | |
| N216Q + S313D + E408D + D476R + | E229S + N440K + S582K + A624E + | 18 | 16 | 92% |
| Y579W + I602T + F638N + A651P + | S635E + N672D + G738L + G753E + | | | |
| T697G + W719R + R880K + T887K + | S754E + S757D + A769D + L775A + | | | |
| K921R + Y934G | D801G + K875T + N892Y | | | |

TABLE 36
Residual wash performance (RP) of XL and EG enzyme combinations (wildtypes or variants) after
storage 13 days in PUG detergent at 30° C. Tested on Fluid Make up (CS-17) swatches.

| Enzyme mutations relative to SEQ ID NO: 2 | | Fluid Make up, CS-17 | | |
|---|---|---|---|---|
| (EG) and relative to SEQ ID NO: 6 (XL) | | Delta Intensity | | |
| Endoglucanase (EG) | Xanthan lyase (XL) | Fresh | Stored | RP |
| none, wildtype | none, wildtype | 9 | 0 | 3% |
| A559N + Y579W + T697G | E229N + N672D + P752K + G753E + | 10 | 3 | 29% |
| | A769D + L775A + D801G + K875T + | | | |
| | N892Y | | | |
| A559N + Y579W + T697G | E229S + N672D + G753E + S754E + | 10 | 4 | 39% |
| | A769D + L775A + D801G + K875T + | | | |
| | N892Y | | | |
| A559N + Y579W + T697G | E229S + N672D + P752R + G753E + | 12 | 6 | 52% |
| | S754E + A769D + L775A + D801G + | | | |
| | K875T + N892Y | | | |
| A559N + Y579W + T697G | A190Q + E229S + I234V + A624E + | 12 | 4 | 32% |
| | N672D + G753E + S754E + A769D + | | | |
| | L775A + D801G + K875T+ | | | |
| A559N + Y579W + T697G | A190Q + E229S + T631N + N672D + | 11 | 5 | 40% |
| | I703L + P752K + G753E + A769D + | | | |
| | L775A + D801G + K875T+ | | | |
| A559N + Y579W + T697G | A190Q + E229S + I234V + S582K + | 12 | 8 | 65% |
| | N672D + G753E + S754E + S757D + | | | |
| | A769D + L775A + D801G + K875T + | | | |
| | N892Y | | | |
| A559N + Y579W + T697G | E229S + N440K + S582K + A624E + | 8 | 5 | 62% |
| | S635E + N672D + G738L + G753E + | | | |
| | S754E + S757D + A769D + L775A + | | | |
| | D801G + K875T + N892Y | | | |
| K512P + A559N + Y579W + T697G | E229S + N672D + G753E + S754E + | 9 | 4 | 49% |
| | A769D + L775A + D801G + K875T + | | | |
| | N892Y | | | |
| K512P + A559N + Y579W + T697G | E229S + N672D + P752R + G753E + | 10 | 4 | 34% |
| | S754E + A769D + L775A + D801G + | | | |
| | K875T + N892Y | | | |
| K512P + A559N + Y579W + T697G | A190Q + E229S + I234V + A624E + | 10 | 4 | 37% |
| | N672D + G753E + S754E + A769D + | | | |
| | L775A + D801G + K875T+ | | | |
| K512P + A559N + Y579W + T697G | A190Q + E229S + T631N + N672D + | 12 | 4 | 33% |
| | I703L + P752K + G753E + A769D + | | | |
| | L775A + D801G + K875T+ | | | |
| K512P + A559N + Y579W + T697G | A190Q + E229S + I234V + S582K + | 10 | 7 | 69% |
| | N672D + G753E + S754E + S757D + | | | |
| | A769D + L775A + D801G + K875T + | | | |
| | N892Y | | | |
| K512P + A559N + Y579W + T697G | E229S + N440K + S582K + A624E + | 10 | 7 | 65% |
| | S635E + N672D + G738L + G753E + | | | |
| | S754E + S757D + A769D + L775A + | | | |
| | D801G + K875T + N892Y | | | |
| N18G + A71E + A186P + E408D + | E229S + N672D + G753E + S754E + | 8 | 3 | 33% |
| Y579W + I602T + A651P + A688G + | A769D + L775A + D801G + K875T + | | | |
| V756Y | N892Y | | | |
| N18G + A71E + A186P + E408D + | E229S + N672D + P752R + G753E + | 8 | 4 | 54% |
| Y579W + I602T + A651P + A688G + | S754E + A769D + L775A + D801G + | | | |
| V756Y | K875T + N892Y | | | |
| N18G + A71E + A186P + E408D + | A190Q + E229S + I234V + A624E + | 8 | 4 | 56% |
| Y579W + I602T + A651P + A688G + | N672D + G753E + S754E + A769D + | | | |
| V756Y | L775A + D801G + K875T+ | | | |
| N18G + A71E + A186P + E408D + | A190Q + E229S + T631N + N672D + | 8 | 5 | 62% |
| Y579W + I602T + A651P + A688G + | I703L + P752K + G753E + A769D + | | | |
| V756Y | L775A + D801G + K875T+ | | | |
| N18G + A71E + A186P + E408D + | A190Q + E229S + I234V + S582K + | 7 | 5 | 76% |
| Y579W + I602T + A651P + A688G + | N672D + G753E + S754E + S757D + | | | |
| V756Y | A769D + L775A + D801G + K875T + | | | |
| | N892Y | | | |
| N18G + A71E + A186P + E408D + | E229S + N440K + S582K + A624E + | 4 | 4 | 81% |
| Y579W + I602T + A651P + A688G + | S635E + N672D + G738L + G753E + | | | |
| V756Y | S754E + S757D + A769D + L775A + | | | |
| | D801G + K875T + N892Y | | | |
| N18G + N189K + E408D + A559N + | E229N + N672D + P752K + G753E + | 6 | 2 | 32% |
| Y579W + A688G + T697G + V756Y + | A769D + L775A + D801G + K875T + | | | |
| K921R + Y934G | N892Y | | | |
| N18G + N189K + E408D + A559N + | E229S + N672D + G753E + S754E + | 6 | 4 | 61% |
| Y579W + A688G + T697G + V756Y + | A769D + L775A + D801G + K875T + | | | |
| K921R + Y934G | N892Y | | | |
| N18G + N189K + E408D + A559N + | E229S + N672D + P752R + G753E + | 8 | 4 | 47% |
| Y579W + A688G + T697G + V756Y + | S754E + A769D + L775A + D801G + | | | |
| K921R + Y934G | K875T + N892Y | | | |
| N18G + N189K + E408D + A559N + | A190Q + E229S + I234V + A624E + | 8 | 3 | 37% |
| Y579W + A688G + T697G + V756Y + | N672D + G753E + S754E + A769D + | | | |
| K921R + Y934G | L775A + D801G + K875T+ | | | |
| N18G + N189K + E408D + A559N + | A190Q + E229S + T631N + N672D + | 7 | 5 | 63% |
| Y579W + A688G + T697G + V756Y + | I703L + P752K + G753E + A769D + | | | |
| K921R + Y934G | L775A + D801G + K875T+ | | | |
| N18G + N189K + E408D + A559N + | A190Q + E229S + I234V + S582K + | 8 | 7 | 87% |
| Y579W + A688G + T697G + V756Y + | N672D + G753E + S754E + S757D + | | | |
| K921R + Y934G | A769D + L775A + D801G + K875T + | | | |
| | N892Y | | | |
| N18G + N189K + E408D + A559N + | E229S + N440K + S582K + A624E + | 7 | 6 | 96% |
| Y579W + A688G + T697G + V756Y + | S635E + N672D + G738L + G753E + | | | |
| K921R + Y934G | S754E + S757D + A769D + L775A + | | | |
| | D801G + K875T + N892Y | | | |
| S313D + E408D | E229N + N672D + P752K + G753E + | 10 | 5 | 49% |
| | A769D + L775A + D801G + K875T + | | | |
| | N892Y | | | |
| S313D + E408D | E229S + N672D + G753E + S754E + | 9 | 4 | 47% |
| | A769D + L775A + D801G + K875T + | | | |
| | N892Y | | | |
| S313D + E408D | E229S + N672D + P752R + G753E + | 10 | 5 | 53% |
| | S754E + A769D + L775A + D801G + | | | |
| | K875T + N892Y | | | |
| S313D + E408D | A190Q + E229S + I234V + A624E + | 10 | 5 | 47% |
| | N672D + G753E + S754E + A769D + | | | |
| | L775A + D801G + K875T+ | | | |
| S313D + E408D | A190Q + E229S + T631N + N672D + | 11 | 5 | 49% |
| | I703L + P752K + G753E + A769D + | | | |
| | L775A + D801G + K875T+ | | | |
| S313D + E408D | A190Q + E229S + I234V + S582K + | 9 | 8 | 91% |
| | N672D + G753E + S754E + S757D + | | | |
| | A769D + L775A + D801G + K875T + | | | |
| | N892Y | | | |
| S313D + E408D | E229S + N440K + S582K + A624E + | 7 | 6 | 83% |
| | S635E + N672D + G738L + G753E + | | | |
| | S754E + S757D + A769D + L775A + | | | |
| | D801G + K875T + N892Y | | | |
| R880K + N905D + K921R + Y934G | E229N + N672D + P752K + G753E + | 8 | 3 | 32% |
| | A769D + L775A + D801G + K875T + | | | |
| | N892Y | | | |
| R880K + N905D + K921R + Y934G | E229S + N672D + G753E + S754E + | 10 | 6 | 57% |
| | A769D + L775A + D801G + K875T + | | | |
| | N892Y | | | |
| R880K + N905D + K921R + Y934G | E229S + N672D + P752R + G753E + | 9 | 2 | 26% |
| | S754E + A769D + L775A + D801G + | | | |
| | K875T + N892Y | | | |
| R880K + N905D + K921R + Y934G | A190Q + E229S + I234V + A624E + | 11 | 5 | 44% |
| | N672D + G753E + S754E + A769D + | | | |
| | L775A + D801G + K875T+ | | | |
| R880K + N905D + K921R + Y934G | A190Q + E229S + T631N + N672D + | 11 | 5 | 45% |
| | I703L + P752K + G753E + A769D + | | | |
| | L775A + D801G + K875T+ | | | |
| R880K + N905D + K921R + Y934G | A190Q + E229S + I234V + S582K + | 11 | 8 | 72% |
| | N672D + G753E + S754E + S757D + | | | |
| | A769D + L775A + D801G + K875T + | | | |
| | N892Y | | | |
| R880K + N905D + K921R + Y934G | E229S + N440K + S582K + A624E + | 10 | 8 | 79% |
| | S635E + N672D + G738L + G753E + | | | |
| | S754E + S757D + A769D + L775A + | | | |
| | D801G + K875T + N892Y | | | |
| I302D + S313D + E408D + Y579W + | E229N + N672D + P752K + G753E + | 10 | 3 | 33% |
| I602T + A651P + T697G + R880K + | A769D + L775A + D801G + K875T + | | | |
| K921R + Y934G | N892Y | | | |
| I302D + S313D + E408D + Y579W + | E229S + N672D + G753E + S754E + | 9 | 2 | 18% |
| I602T + A651P + T697G + R880K + | A769D + L775A + D801G + K875T + | | | |
| K921R + Y934G | N892Y | | | |
| I302D + S313D + E408D + Y579W + | E229S + N672D + P752R + G753E + | 9 | 5 | 55% |
| I602T + A651P + T697G + R880K + | S754E + A769D + L775A + D801G + | | | |
| K921R + Y934G | K875T + N892Y | | | |
| I302D + S313D + E408D + Y579W + | A190Q + E229S + I234V + A624E + | 10 | 4 | 35% |
| I602T + A651P + T697G + R880K + | N672D + G753E + S754E + A769D + | | | |
| K921R + Y934G | L775A + D801G + K875T+ | | | |
| I302D + S313D + E408D + Y579W + | A190Q + E229S + T631N + N672D + | 10 | 5 | 47% |
| I602T + A651P + T697G + R880K + | I703L + P752K + G753E + A769D + | | | |
| K921R + Y934G | L775A + D801G + K875T+ | | | |
| I302D + S313D + E408D + Y579W + | A190Q + E229S + I234V + S582K + | 9 | 6 | 61% |
| I602T + A651P + T697G + R880K + | N672D + G753E + S754E + S757D + | | | |
| K921R + Y934G | A769D + L775A + D801G + K875T + | | | |
| | N892Y | | | |
| I302D + S313D + E408D + Y579W + | E229S + N440K + S582K + A624E + | 7 | 9 | 120% |
| I602T + A651P + T697G + R880K + | S635E + N672D + G738L + G753E + | | | |
| K921R + Y934G | S754E + S757D + A769D + L775A + | | | |
| | D801G + K875T + N892Y | | | |
| N216Q + S313D + E408D + D476R + | E229N + N672D + P752K + G753E + | 8 | 8 | 94% |
| Y579W + I602T + F638N + A651P + | A769D + L775A + D801G + K875T + | | | |
| T697G + W719R + R880K + T887K + | N892Y | | | |
| K921R + Y934G | | | | |
| N216Q + S313D + E408D + D476R + | E229S + N672D + G753E + S754E + | 10 | 9 | 92% |
| Y579W + I602T + F638N + A651P + | A769D + L775A + D801G + K875T + | | | |
| T697G + W719R + R880K + T887K + | N892Y | | | |
| K921R + Y934G | | | | |
| N216Q + S313D + E408D + D476R + | E229S + N672D + P752R + G753E + | 9 | 10 | 117% |
| Y579W + I602T + F638N + A651P + | S754E + A769D + L775A + D801G + | | | |
| T697G + W719R + R880K + T887K + | K875T + N892Y | | | |
| K921R + Y934G | | | | |
| N216Q + S313D + E408D + D476R + | A190Q + E229S + I234V + A624E + | 10 | 9 | 94% |
| Y579W + I602T + F638N + A651P + | N672D + G753E + S754E + A769D + | | | |
| T697G + W719R + R880K + T887K + | L775A + D801G + K875T+ | | | |
| K921R + Y934G | | | | |
| N216Q + S313D + E408D + D476R + | A190Q + E229S + T631N + N672D + | 10 | 9 | 93% |
| Y579W + I602T + F638N + A651P + | I703L + P752K + G753E + A769D + | | | |
| T697G + W719R + R880K + T887K + | L775A + D801G + K875T+ | | | |
| K921R + Y934G | | | | |
| N216Q + S313D + E408D + D476R + | A190Q + E229S + I234V + S582K + | 9 | 11 | 122% |
| Y579W + I602T + F638N + A651P + | N672D + G753E + S754E + S757D + | | | |
| T697G + W719R + R880K + T887K + | A769D + L775A + D801G + K875T + | | | |
| K921R + Y934G | N892Y | | | |
| N216Q + S313D + E408D + D476R + | E229S + N440K + S582K + A624E + | 10 | 13 | 125% |
| Y579W + I602T + F638N + A651P + | S635E + N672D + G738L + G753E + | | | |
| T697G + W719R + R880K + T887K + | S754E + S757D + A769D + L775A + | | | |
| K921R + Y934G | D801G + K875T + N892Y | | | |

### Example 11

### Washing Test

TABLE 37
Liquid laundry detergent matrix:

| | % Active Matter | % Active Matter |
|---|---|---|
| | raw material | in Formula |
| Water demin. | 100 | ad 100% |
| Protease Stabilisator | 100 | 0.8-1.2% |
| Citric acid | 100 | 2.5-4% |
| Antifoaming agent | t.q. | 0.02-0.05% |
| FAEOS (anionic surfactant) | 70 | 7.5-9.5% |
| FAEO (nonionic surfactant) | 100 | 5.5-7.5% |
| LAS (anionic surfactant) | 96 | 6-8% |
| palm kernel oil fatty acid | 30 | 2.5-4.5% |
| NaOH | 50 | 2.5-3.5% |
| 1,2-Propandiol | 100 | 4-7% |
| DTPMP-xNa | 40 | 0.5-1% |
| Soil repellent | 70 | 1-1.8% |
| Ethanol | 93 | 1-4% |
| Optical brightener | 90 | 0.05-2% |
| Enzyme Mix | 100 | 1.4-1.8% |
| Perfume | 100 | 0.5-1.4% |
| Dye | tq | 0.001-0.004% |
| Dosage: 73 mL | | |
| pH: 8.2-8.6 | | |

The enzymes to be tested were combined with the above liquid laundry detergent matrix in the concentrations given below, homogenized and stored in a closed container at 30° C. for 8 weeks. The freshly prepared matrix including the enzymes and the matrix with the enzymes stored for 8 weeks at 30° C. were used in the following wash test:

The wash test was performed under standard conditions (40° C., color program, 16° dH) and the brightness of several different commercial stains determined after drying and ironing via brightness measurement. To the stain sets, 3.5 kg clean cotton textiles and 4 SBL sheets were added.

Stain Set:

| | |
|---|---|
| CFT CS44 [CO] | Chocolate drink |
| CFT CS06 [CO] | Salad dressing |
| CFT CS05S [CO] | Mayonaise |
| CFT CS02 [CO] | Cocoa |
| EMPA 165 [CO] | chocolate pudding |

Every wash test was performed 6 times and the average values calculated.

The enzymes were used in active protein concentrations of 102 mg xanthan lyase and 1.53 mg endoglucanase. The following enzyme combinations were tested:

Combination WT: Xanthan lyase WT (SEQ ID NO:6) and endoglucanase WT (SEQ ID NO:2)

Combination 1: Xanthan lyase variant 1 (SEQ ID NO:6 with the following substitutions: A190Q+E229S+I234V+S582K+N672D+G753E+S754E+S757D+A769D+L775A+D801G+K875T+N89 2Y) and endoglucanase variant 2 (SEQ ID NO:2 with the following substitutions: N216Q+5313D+E408D+D476R+Y579W+I602T+F638N+A651P+T697G+W719R+R880K+T887K+K9 21R+Y934G)

Combination 2: Xanthan lyase variant 2 (SEQ ID NO:6 with E229S+N440K+S582K+A624E+S635E+N672D+G738L+G753E+S754E+S757D+A769D+L775A+D80 1G+K875T+N892Y) and endoglucanase variant 1 (SEQ ID NO:2 with F20P+S313D+E408D+Y579W+S636K+A688G+T697G+N905D+A937E)

Combination 3: Xanthan lyase variant 3 (SEQ ID NO:6 with S100D+E229S+K360G+D458S+S582K+N672D+G753E+S754E+S757D+A769D+L775A+D801G+A8 43P+K875T+N892Y+N1008D) and endoglucanase variant 2 (SEQ ID NO:2 with N216Q+S313D+E408D+D476R+Y579W+I602T+F638N+A651P+T697G+W719R+R880K+T887K+K9 21R+Y934G)

In the following table Delta Y values of the inventive formulations are given relative to the detergent without enzymes, each as the sum of the 5 given stains. The higher the value the better the cleaning performance of the respective enzyme combination.

TABLE 38
Washing test results

| Enzyme | Performance | Performance after |
|---|---|---|
| combination | fresh | 8 weeks at 30° C. |
| WT | 26.2 | 0 |
| 1 | 26.4 | 16.6 |
| 2 | 26.6 | 17.9 |
| 3 | 22.1 | 16.0 |

As can be seen from the results, the enzyme combinations of the invention provide for a significantly better performance after extended storage at 30° C.

1. A detergent composition comprising:
(A) an endoglucanase variant comprising an alteration at one or more positions in a region selected from the group consisting of: region 1 corresponding to amino acids 95 to 105 of SEQ ID NO: 2, region 2 corresponding to amino acids 115 to 138 of SEQ ID NO: 2, region 3 corresponding to amino acids 210 to 251 of SEQ ID NO: 2, region 4 corresponding to amino acids 267 to 301 of SEQ ID NO: 2, region 5 corresponding to amino acids 339 to 361 of SEQ ID NO: 2, region 6 corresponding to amino acids 547 to 595 of SEQ ID NO: 2, region 7 corresponding to amino acids 612 to 660 of SEQ ID NO: 2, region 8 corresponding to amino acids 806 to 828 of SEQ ID NO: 2, region 9 corresponding to amino acids 839 to 1042 of SEQ ID NO: 2, region 10 corresponding to amino acids 1 to 94 of SEQ ID NO: 2, region 11 corresponding to amino acids 106 to 114 of SEQ ID NO: 2, region 12 corresponding to amino acids 139 to 209 of SEQ ID NO: 2, region 13 corresponding to amino acids 252 to 266 of SEQ ID NO: 2, region 14 corresponding to amino acids 302 to 338 of SEQ ID NO: 2, region 15 corresponding to amino acids 362 to 546 of SEQ ID NO: 2, region 16 corresponding to amino acids 596 to 611 of SEQ ID NO: 2, region 17 corresponding to amino acids 661 to 805 of SEQ ID NO: 2, region 18 corresponding to amino acids 829 to 838 of SEQ ID NO: 2, and region 19 corresponding to amino acids 1043 to 1055 of SEQ ID NO: 2; and
(B) a xanthan lyase variant comprising an alterationat one or more positions in a region selected from the group consisting of: region 1 corresponding to amino acids 154 to 176 of SEQ ID NO: 6, region 2 corresponding to amino acids 614 to 658 of SEQ ID NO: 6, region 3 corresponding to amino acids 731 to 803 of SEQ ID NO: 6, region 4 corresponding to amino acids 807 to 846 of SEQ ID NO: 6, region 5 corresponding to amino acids 872 to 885 of SEQ ID NO: 6, region 6 corresponding to amino acids 903 to 1004 of SEQ ID NO: 6, region 7 corresponding to amino acids 1 to 153 of SEQ ID NO: 6, region 8 corresponding to amino acids 177 to 613 of SEQ ID NO: 6, region 9 corresponding to amino acids 659 to 730 of SEQ ID NO: 6, region 10 corresponding to amino acids 804 to 806 of SEQ ID NO: 6, region 11 corresponding to amino acids 847 to 871 of SEQ ID NO: 6, region 12 corresponding to amino acids 886 to 902 of SEQ ID NO: 6, and region 13 corresponding to amino acids 1005 to 1037 of SEQ ID NO: 6;
wherein the endoglucanase variant (A) has at least 60% and less than 100% sequence identity to SEQ ID NO: 2; and wherein the xanthan lyase variant (B) has at least 60% and less than 100% sequence identity to SEQ ID NO.

2. The detergent composition according to claim 1, wherein said endoglucanase variant comprises an alteration at one or more positions in a region selected from the group consisting of:
i) region 1 corresponding to amino acids 95 to 105 of SEQ ID NO: 2, said alteration being at one or more positions selected from the group consisting of positions: 95, 96, 97, 98, 99, 100, 101, 102, 103, 104, 105, wherein said positions correspond to amino acid positions of SEQ ID NO: 2 according to the numbering of SEQ ID NO: 2,
ii) region 2 corresponding to amino acids 115 to 138 of SEQ ID NO: 2, said alteration being at one or more positions selected from the group consisting of positions: 115, 116, 117, 118, 119, 120, 121, 122, 123, 124, 125, 126, 127, 128, 129, 130, 131, 132, 133, 134, 135, 136, 137, 138, wherein said positions correspond to amino acid positions of SEQ ID NO: 2 according to the numbering of SEQ ID NO: 2,
iii) region 3 corresponding to amino acids 210 to 251 of SEQ ID NO: 2, said alteration being at one or more positions selected from the group consisting of positions: 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, wherein said positions correspond to amino acid positions of SEQ ID NO: 2 according to the numbering of SEQ ID NO: 2,
iv) region 4 corresponding to amino acids 267 to 301 of SEQ ID NO: 2, said alteration being at one or more positions selected from the group consisting of positions: 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, wherein said positions correspond to amino acid positions of SEQ ID NO: 2 according to the numbering of SEQ ID NO: 2,
v) region 5 corresponding to amino acids 339 to 361 of SEQ ID NO: 2, said alteration being at one or more positions selected from the group consisting of positions: 339, 340, 341, 342, 343, 344, 345, 346, 347, 348, 349, 350, 351, 352, 353, 354, 355, 356, 357, 358, 359, 360, 361, wherein said positions correspond to amino acid positions of SEQ ID NO: 2 according to the numbering of SEQ ID NO: 2,
vi) region 6 corresponding to amino acids 547 to 595 of SEQ ID NO: 2, said alteration being at one or more positions selected from the group consisting of positions: 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, wherein said positions correspond to amino acid positions of SEQ ID NO: 2 according to the numbering of SEQ ID NO: 2,
vii) region 7 corresponding to amino acids 612 to 660 of SEQ ID NO: 2, said alteration being at one or more positions selected from the group consisting of positions: 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, wherein said positions correspond to amino acid positions of SEQ ID NO: 2 according to the numbering of SEQ ID NO: 2,
viii) region 8 corresponding to amino acids 806 to 828 of SEQ ID NO: 2, said alteration being at one or more positions selected from the group consisting of positions: 806, 807, 808, 809, 810, 811, 812, 813, 814, 815, 816, 817, 818, 819, 820, 821, 822, 823, 824, 825, 826, 827, 828, wherein said positions correspond to amino acid positions of SEQ ID NO: 2 according to the numbering of SEQ ID NO: 2,
ix) region 9 corresponding to amino acids 839 to 1042 of SEQ ID NO: 2, said alteration being at one or more positions selected from the group consisting of positions: 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, wherein said positions correspond to amino acid positions of SEQ ID NO: 2 according to the numbering of SEQ ID NO: 2,
x) region 10 corresponding to amino acids 1 to 94 of SEQ ID NO: 2, said alteration being at one or more positions selected from the group consisting of positions: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, wherein said positions correspond to amino acid positions of SEQ ID NO: 2 according to the numbering of SEQ ID NO: 2,
xi) region 11 corresponding to amino acids 106 to 114 of SEQ ID NO: 2, said alteration being at one or more positions selected from the group consisting of positions: 107, 108, 109, 110, 111, 112, 113, 114, wherein said positions correspond to amino acid positions of SEQ ID NO: 2 according to the numbering of SEQ ID NO: 2[[)]],
xii) region 12 corresponding to amino acids 139 to 209 of SEQ ID NO: 2, said alteration being at one or more positions selected from the group consisting of positions: 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, wherein said positions correspond to amino acid positions of SEQ ID NO: 2 according to the numbering of SEQ ID NO: 2,
xiii) region 13 corresponding to amino acids 252 to 266 of SEQ ID NO: 2, said alteration being at one or more positions selected from the group consisting of positions: 252, 253, 254, 255, 256, 257, 258, 259, 260, 261, 262, 263, 264, 265, 266, wherein said positions correspond to amino acid positions of SEQ ID NO: 2 according to the numbering of SEQ ID NO: 2,
xiv) region 14 corresponding to amino acids 302 to 338 of SEQ ID NO: 2, said alteration being at one or more positions selected from the group consisting of positions: 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, wherein said positions correspond to amino acid positions of SEQ ID NO: 2 according to the numbering of SEQ ID NO: 2,
xv) region 15 corresponding to amino acids 362 to 546 of SEQ ID NO: 2, said alteration being at one or more positions selected from the group consisting of positions: 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482 ,483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, wherein said positions correspond to amino acid positions of SEQ ID NO: 2 according to the numbering of SEQ ID NO: 2,
xvi) region 16 corresponding to amino acids 596 to 611 of SEQ ID NO: 2, said alteration being at one or more positions selected from the group consisting of positions: 596, 597, 598, 599, 600, 601, 602, 603, 604, 605, 606, 607, 608, 609, 610, 611, wherein said positions correspond to amino acid positions of SEQ ID NO: 2 according to the numbering of SEQ ID NO: 2,
xvii) region 17 corresponding to amino acids 661 to 805 of SEQ ID NO: 2, said alteration being at one or more positions selected from the group consisting of positions: 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, , 781, 782, 783, 784, 785, 786, 787, 788, 789, 790, 791, 792, 793, 794, 795, 796, 797, 798, 799, 800, 801, 802, 803, 804, 805, wherein said positions correspond to amino acid positions of SEQ ID NO: 2 according to the numbering of SEQ ID NO: 2,
xviii) region 18 corresponding to amino acids 829 to 838 to 1042 of SEQ ID NO: 2, said alteration being at one or more positions selected from the group consisting of positions: 829, 830, 831, 832, 833, 834, 835, 836, 837, 838, wherein said positions correspond to amino acid positions of SEQ ID NO: 2 according to the numbering of SEQ ID NO: 2, and
xix) region 19 corresponding to amino acids 1043 to 1055 of SEQ ID NO: 2, said alteration being at one or more positions selected from the group consisting of positions 1043, 1044, 1045, 1046, 1047, 1048, 1049, 1050, 1051, 1052, 1053, 1054, 1055, wherein said positions correspond to amino acid positions of SEQ ID NO: 2 according to the numbering of SEQ ID NO: 2.

3. The detergent composition according to claim 1, wherein said xanthan lyase variant comprises an alteration at one or more positions in a region selected from the group consisting of:
i) region 1 corresponding to amino acids 154 to 176 of SEQ ID NO: 6, said alteration being at one or more positions selected from the group consisting of positions: 154, 155, 156, 157, 158, 159, 160, 161, 162, 163, 164, 165, 166, 167, 168, 169, 170, 171, 172, 173, 174, 175, 176, wherein said positions correspond to amino acid positions of SEQ ID NO: 6 according to the numbering of SEQ ID NO: 6,
ii) region 2 corresponding to amino acids 614 to 658 of SEQ ID NO: 6, said alteration being at one or more positions selected from the group consisting of positions: 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, wherein said positions correspond to amino acid positions of SEQ ID NO: 6 according to the numbering of SEQ ID NO: 6,
iii) region 3 corresponding to amino acids 731 to 803 of SEQ ID NO: 6, said alteration being at one or more positions selected from the group consisting of positions: 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, , 781, 782, 783, 784, 785, 786, 787, 788, 789, 790, 791, 792, 793, 794, 795, 796, 797, 798, 799, 800, 801, 802, 803, wherein said positions correspond to amino acid positions of SEQ ID NO: 6 using according to the numbering of SEQ ID NO: 6,
iv) region 4 corresponding to amino acids 807 to 846 of SEQ ID NO: 6, said alteration being at one or more positions selected from the group consisting of positions: 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, wherein said positions correspond to amino acid positions of SEQ ID NO: 6 according to the numbering of SEQ ID NO: 6,
v) region 5 corresponding to amino acids 872 to 885 of SEQ ID NO: 6, said alteration being at one or more positions selected from the group consisting of positions: 872, 873, 874, 875, 876, 877, 878, 879, 880, 881, 882, 883, 884, 885, wherein said positions correspond to amino acid positions of SEQ ID NO: 6 according to the numbering of SEQ ID NO: 6,
vi) region 6 corresponding to amino acids 903 to 1004 of SEQ ID NO: 6, said alteration being at one or more positions selected from the group consisting of positions: 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, wherein said positions correspond to amino acid positions of SEQ ID NO: 6 according to the numbering of SEQ ID NO: 6[[)]],
vii) region 7 corresponding to amino acids 1 to 153 of SEQ ID NO: 6, said alteration being at one or more positions selected from the group consisting of positions: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152 and 153, wherein said positions correspond to amino acid positions of SEQ ID NO: 6 according to the numbering of SEQ ID NO:6,
viii) region 8 corresponding to amino acids 177 to 613 of SEQ ID NO: 6, said alteration being at one or more positions selected from the group consisting of positions:
177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612 and 613, wherein said positions correspond to amino acid positions of SEQ ID NO: 6 according to the numbering of SEQ ID NO:6,
ix) region 9 corresponding to amino acids 659 to 730 of SEQ ID NO: 6, said alteration being at one or more positions selected from the group consisting of positions: 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729 and 730, wherein said positions correspond to amino acid positions of SEQ ID NO: 6 according to the numbering of SEQ ID NO:6,
x) region 10 corresponding to amino acids 804 to 806 of SEQ ID NO: 6, said alteration being at one or more positions selected from the group consisting of positions: 804, 805 and 806, wherein said positions correspond to amino acid positions of SEQ ID NO: 6 according to the numbering of SEQ ID NO:6,
xi) region 11 corresponding to amino acids 847 to 871 of SEQ ID NO: 6, said alteration being at one or more positions selected from the group consisting of positions: 847, 848, 849, 850, 851, 852, 853, 854, 855, 856, 857, 858, 859, 860, 861, 862, 863, 864, 865, 866, 867, 868, 869, 870 and 871, wherein said positions correspond to amino acid positions of SEQ ID NO: 6 according to the numbering of SEQ ID NO:6,
xii) region 12 corresponding to amino acids 886 to 902 of SEQ ID NO: 6, said alteration being at one or more positions selected from the group consisting of positions: 886, 887, 888, 889, 890, 891, 892, 893, 894, 895, 896, 897, 898, 899, 900, 901 and 902, wherein said positions correspond to amino acid positions of SEQ ID NO: 6 according to the numbering of SEQ ID NO:6, and
xiii) region 13 corresponding to amino acids 1005 to 1037 of SEQ ID NO: 6, said alteration being at one or more positions selected from the group consisting of positions: 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036 and 1037, wherein said positions correspond to amino acid positions of SEQ ID NO: 6 according to the numbering of SEQ ID NO:6.

4. The detergent composition according to claim 1, wherein the endoglucanase variant has an alteration in the positions selected from the group consisting of positions: 559+579+697; 512+559+579+697; 18+71+186+408+579+602+651+688+756; 18+189+408+559+579+688+697+756+921+934; 313+488; 880+905+921+934; 302+313+408+579+602+651+697+880+921+934; or 216+313+408+476+579+602+638+651+697+719+880+887+921+934 of SEQ ID NO: 2.

5. The detergent composition according to claim 1, wherein the xanthan lyase variant has an alteration in the positions selected from the group consisting of positions: 190, 229, 234, 440, 582, 624, 631, 635, 672, 703, 738, 752, 753, 754, 757, 769, 775, 801, 875, 892, and any combination thereof.

6. The detergent composition according to claim 1, wherein the detergent composition comprises:
(A) an endoglucanase variant selected from those having at least 61%, sequence identity to SEQ ID NO: 2 and having an alteration, in the positions selected from the group consisting of positions: 559+579+697; 512+559+579+697; 18+71+186+408+579+602+651+688+756; 18+189+408+559+579+688+697+756+921+934; 313+488; 880+905+921+934; 302+313+408+579+602+651+697+880+921+934; or 216+313+408+476+579+602+638+651+697+719+880+887+921+934 of SEQ ID NO: 2;
and
(B) a xanthan lyase variant having at least 61%, sequence identity to SEQ ID NO: 6 and having an alteration, in the positions selected from the group consisting of positions: 229+672+752+753+769+775+801+875+892; 229+672+753+754+769+775+801+875+892; 229+672+752+753+754+769+775+801+875+892; 190+229+234+624+672+753+754+769+775+801+875; 190+229+631+672+703+752+753+769+775+801+875; 190+229+234+582+672+753+754+757+769+775+801+875+892; 229+440+582+624+635+672+738+753+754+757+769+775+801+875+892; or 100+229+360+458+582+672+753+754+757+769+775+801+843+875+892+1008 of SEQ ID NO: 6;
and
(B 8) S100D+E229S+K360G+D458S+S582K+N672D+G753E+S754E+S757D+A769D+L775 A+D801G+A843P+K875T+N892Y+N1008D of SEQ ID NO: 6.

7. The detergent composition according to claim 6, wherein the endoglucanase and/or the xanthan lyase variant do not comprise any further substitution.

8. The detergent composition according to claim 1, wherein the detergent composition additionally comprises one or more further detergent components.

9. (canceled)

10. A method of degrading xanthan gum by contacting the xanthan gum with a detergent composition according to claim 1, wherein the xanthan gum is disposed on a surface of a hard surface or textile.


SEQUENCE LISTING (6 sequences)

SEQ ID NO: 1 - DNA, 12110 bp, Paenibacillus sp-62047
tctggttgttttcttcatttcaggtttcgccctttccttgccaaatataagaaaaacggcgttccgataatcgcggtgacggtgattcataaggaaatgcaatccatctggccagaacatctgcgtacaccagcaaaatggcaccgaacagtgccgaaaacggaagcacgtattgataatgttctccgatcagcttgcggacaatatgcgggacgagcagcccgacaaagccaatcggcccggcgacggctacggaagcgccggaaagaattaaaataatcaaactgatcagaatcctgatgccgttcatattttgtccaagcccttttgctgtttcgtctccgagaccgagaacagaaacagaaccggaaaatacgagggcaagcccgatgccaatgacagaaaaaggagcgatggttatgacgtcctgccagttgctgccgtcgattgcgcctgtcatccagtacagaacatcctcacctgactcatttaaaataatgatggcctgtgtcatagaggagaggaacaagtgcacggccattcctgacagcgccagcttgacaggcgtcattccgccggatgaggcaatcatatacacaatcgcgccgcctgctgccgcacccgcaaaagcgaatataacagatgaatagggcgatgccggcagaatgacgagagaagcaacaacaaaaagcgatgcacccgcattcacaccgaaaatttggggtgaagccagaggatttctggtcatagcctgcatcagcgcccctgctacagctaggctggcgccgacaaaaacgccgattaatgtgcggggaaggcgaagagtagagatgatgagctgttcctttgaaccgtcccatacaaaaagatatttcaatgaatctatgatgctgatgtctgaggctcctactgaaagattcagcccaagcccaaatataaaaataatcagtgcaatgataaacatcatcagtcttgatgatgagcgccgtttggctgaatgatacaacagtctcacttccttactgcgtctggttgcaaaaacgaagaagcaaggattcccctcgcttctcatttgtcctatttattatacacttttttaagcacatctttggcgcttgtttcactagacttgatgcctctgaatcttgtccaagtgtcacggtccgcatcatagacttgtccatttttcaccgctttgagatttttccagagcgggttcgttttccactcatctacaatggttttgccttcgttggctgagatgaacaaaatatcaggatcgattttgctcaattgctcaaggctgacctcttgataggcgttatctgacttcacagcgtgtgtaaagcctagcattttaaagatttctccgtcataggatgatgatgtatgaagctggaaggaatccgctcttgcaacgccgagaacgatgttgcggttttcatctttcggaagttcggcttttagatcgttgatgacttttttgtgctcggcaagcttttcttttccttcatcttctttatttaatgctttagcaatggtcgtaaagctgtcgatcgtttcgtcatatgtcgcttcacggctttttaattcaatcgtcggggcgatttttttcagctgtttataaatgtttttatggcgctcagcgtcagcgatgattaaatcaggcttcaaggaactgatgacctcaagattgggttcgctgcgtgtgcctacagatgtgtaatcaatggagctgccgacaagctttttaatcatatcttttttgttgtcatctgcgatgcccaccggcgtaatgccgagattgtgaacggcatccaagaatgaaagctcaagcacaaccacccgcttaggtgtgccgcttactgtcgtttttccttcttcgtcatggatcactctggaatccttagactcgcttttgccgcttccgttgttattctggcttgatgaacagccggatacaatgaggcaggcgagcaataaaacactcatgatggcaatcaacttgttagaataggtgcgcatgtcattcttccttttttcagatttagtaatgagaatcattatcacatgtaacactataatagcatggcttatcatgtcaatatttttttagtaaagaaagctgcgtttttactgctttctcatgaaagcatcatcagacacaaataagtggtatgcagcgttaccgtgtcttcgagacaaaaacgcatgggcgttggctttagaggtttcgaacatatcagcagtgacataaggaaggagagtgctgagataaccggacaatttcttttctatttcatctgttagtgcaaattcaatgtcgccgatattcatgataatcgagaaaacaaagtcgatatcgatatgaaaatgttcctcggcaaaaaccgcaagctcgtgaattcctggtgaacatccggcacgcttatggaaaatctgtttgactaaatcactcacaatccaagcattgtattgctgttctggtgaaaagtattgcattagacatacctcctgctcgtacggataaaggcagcgtttcatggtcgtgtgctccgtgcagcggcttctccttaattttgatttttctgaaaataggtcccgttcctatcactttaccatggacggaaaacaaatagctactaccattcctcctgtttttctcttcaatgttctggaatctgtttcaggtacagacgatcgggtatgaaagaaatatagaaaacatgaaggaggaatatcgacatgaaaccagttgtaaaagagtatacaaatgacgaacagctcatgaaagatgtagaggaattgcagaaaatgggtgttgcgaaagaggatgtatacgtcttagctcacgacgatgacagaacggaacgcctggctgacaacacgaacgccaacacgatcggagccaaagaaacaggtttcaagcacgcggtgggaaatatcttcaataaaaaaggagacgagctccgcaataaaattcacgaaatcggtttttctgaagatgaagccgctcaatttgaaaaacgcttagatgaaggaaaagtgcttctctttgtgacagataacgaaaaagtgaaagcttgggcataaagcaaggaaaaaaccaaaaggccaatgtcggccttttggtttttttgcggtctttgcggtgggattttgcagaatgccgcaataggatagcggaacattttcggttctgaatgtccctcaatttgctattatatttttgtgataaattggaataaaatctcacaaaatagaaaatgggggtacatagtggatgaaaaaagtgatgttagctacggctttgtttttaggattgactccagctggcgcgaacgcagctgatttaggccaccagacgttgggatccaatgatggctggggcgcgtactcgaccggcacgacaggcggatcaaaagcatcctcctcaaatgtgtataccgtcagcaacagaaaccagcttgtctcggcattagggaaggaaacgaacacaacgccaaaaatcatttatatcaagggaacgattgacatgaacgtggatgacaatctgaagccgcttggcctaaatgactataaagatccggagtatgatttggacaaatatttgaaagcctatgatcctagcacatggggcaaaaaagagccgtcgggaacacaagaagaagcgagagcacgctctcagaaaaaccaaaaagcacgggtcatggtggatatccctgcaaacacgacgatcgtcggttcagggactaacgctaaagtcgtgggaggaaacttccaaatcaagagtgataacgtcattattcgcaacattgaattccaggatgcctatgactattttccgcaatggttgtaaaacgacggccagtgaattctgatcaaatggttcagtgagagcgaagcgaacacttgattttttaattttctatcttttataggtcattagagtatacttatttgtcctataaactatttagcagcataatagatttattgaataggtcatttaagttgagcatattagaggaggaaaatcttggagaaatatttgaagaacccgaggatctagatcaggtaccgcaacgttcgcagatgctgctgaagagattattaaaaagctgaaagcaaaaggctatcaattggtaactgtatctcagcttgaagaagtgaagaagcagagaggctattgaataaatgagtagaaagcgccatatcggcgcttttcttttggaagaaaatatagggaaaatggtacttgttaaaaattcggaatatttatacaatatcatatgtatcacattgaaaggaggggcctgctgtccagactgtccgctgtgtaaaaaaaaggaataaaggggggttgacattattttactgatatgtataatataatttgtataagaaaatggaggggccctcgaaacgtaagatgaaaccttagataaaagtgctttttttgttgcaattgaagaattattaatgttaagcttaattaaagataatatctttgaattgtaacgcccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatttatgaaaaatcattttatcagtttgaaaattatgtattatggagctctataaaaatgaggagggaaccgaatgaaacaacaaaaacggctttacgcccgattgctgacgctgttatttgcgctcatcttcttgctgcctcattctgcagcagcagcgatcgcaggcgtggttcaaagcgtgaatgtctcccaagctggttatagttcaaacgattttaagacggctacggtgactgcgtcggataaactaagcgatactagctaccagatattgcaaggcactaccgttatcgcaaccggtacgatgaaggatgaaggatatgtatggggcaaatatgtgtactcgatcgatttctcctccgttacagctacgggcaccaactttacgatccgtagcaacggagtttcatcttacacgttccctatccaaaccaatatgtggaatgaatataaggatgaaatgaccgcgttctaccgtttgctccggactacggatacctttgcggcctatcctgcagggtacagcaatattgcgccttcgaataaaatattacatccggattctttccttgatgatgctttttcgccggaccgaacgacgcattatgacctgactggcggttggttcgatgcaggagactacggtaagtatggcggcaatcagtgggtacagggaaatatcgccatctcttatctgcggcatgcctcttcggcggcggtcaatttcgataaggataccaacggcattccggatctggtggatgaagcgatctttggtagtcagtacttggtgaagtttgccaatcagcttggcggggccattcataatattttgaggaaaggcggctttgtgcttccgcataaagtgacagacaatgttccgggtaacacagacgaccgagcgctcgaagctgtcgaagcggtgggaggctccgggaaatcctctggctcgctggcggcaacggcgcgagcgattcgcactgccatcgcgggcggtaaagtggcagcgaataaagtcgcccagctgcagacacttgcgaatgagtttcaggctgccgcaatcatcttctataattacacattgactcatcaaagtggaaaccatggctcctatggaacaatgaacaatggagggattgcaaatcctctcttatgggcggaagtacagttgtatctgttaacaggggacgctgcatacaagacgcaagctcagacacgcattaatgcaataaatgaagcctatgtttcgtccacgaattattgggatatgcatccgattgcgctggccgaattttattcctgaagcaccaagcatattatttcatcacgctgatggatgaaacgccatacggtgtattgaaccaattcggcaattttggtgtgaatgagccgcatgcatcgtatatggccgatttgctccgatattatgaattgtttaacgatccggtagcgcttcgagcggcgaagaaggcgctgtactggattgtcggcaacaatccatggaatatcagctgggtatccggtgtcggctccaacttcaccgatttcctgcacactcgtctggatgaagaagcatacagccagacgaatacaggcgttgttctgcctggagccatggttagcggaccgaatatcaaagatccgaataacaaattgagctctagcccttggtatgaggataaacctatttgggcagatgacaccaatcaatggagatacaacgaatatagtgtcagtatacagacgggattattctacaccatcatgggcttgtcggcccttggcggaaatgcatccactggtggcgcggagcccgttaagctgccgatcacttggcctatcattggggattatgtgactggggatgtgacggtattcgcacagccggaaggcagcttgagcaatgtgtcagcgaacggaatcgtcttgagcccctccgacggtgtctatacgacgacagtaagcacaagcgctgatgcaccatataccgaaagaaaagtacagatcaaagggacggacgacagcggattcaccacttatagcaatacacatttcacggtggcgcctgcacttccggatccatctcatcctttacttttcgatgactttaaccagaaaggaatctggggtagccaaaagctggattgggtgaattggtataaccaaaacggaggtacagcatcctacacgcggacgacagtggatacaagaacagttgggaaatttgcacatacccctgcagccactacatccaaagccaaattccagccgtggaaatacaacgcaaatcttaacggatatcgctatcttaatttcaccatgaagaatccgggttatcccaataccaaaattcggatagcagcgaatgacggcaccaaatcagttaaccttacgagcggcgaggttgcgatctcgagcacgtggacaacgtatcaatatgatttgaatctgcatccgacgctgaacaagagcaacgttctgattgaggtatggctcagcaacccaactgcgggggcatatggggaaattctcattgacgaaatctcggctgtgaatacgaacagcggaacggcgccaaccttatccgccacaggtgtgaacgcctcgatcggtaatcagtcgacggtatttacttatacagcgacctacaccgatgctaataatcaagcgccgtttgatgtccaggtcgtcattgacggcgtcatccgttcgatgacggcggcggatcctactgacactacttattccgatgggagagtgtatacgtacgctactaccttgccggtggggacgcataagttttacttccggacgacagatacaaccacgaacttcgtcagcacttccgtgcaaaccggaccaaccgtcattcggaatatacacatgctgtaaaagacaatgcggatgcgagcggagggaagtatcgcttgttcaatggtcggcaggccaacgattatattgaatatgcggtgaatgtccctaaggctggaacatatcaagtatctgccagagccatgagattaagcgacaatgggatctaccagctgcagattaacggcagcaatcaaggtactccgttcgatacttaccagtcatcggggaagtatcttgactatgctcttggaaatgtgaccataactagcccgggaacgcagttatttcgattcaaagtaacgggcaaaaatgcaagctcactcggatataagctgccgcttgatttcattcagcttgttccagttccgtaaacgcgttaatcaataaaaaaacgctgtgcggttaaagggcacagcgtttttttgtgtatcggcaatagttacccttattatcaagataagaaagaaaaggatttttcgctacgctcaaatcctttaaaaaaacacaaaagaccacattttttaatgtggtctttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaacgatacattaataggtacgaaaaagcaactttttttgcgcttaaaaccagtcataccaataacttaagggtaactagcctcgccggaaagagcgaaaatgcctcacatttgtgccacctaaaaaggagcgatttacatatgagttatgcagtttgtagaatgcaaaaagtgaaatcagctggactaaaaggggccgcagagtagaatggaaaaggggatcggaaaacaagtatataggaggagacctatttatggcttcagaaaaagacgcaggaaaacagtcagcagtaaagcttgttccattgcttattactgtcgctgtgggactaatcatctggtttattcccgctccgtccggacttgaacctaaagcttggcatttgtttgcgatttttgtcgcaacaattatcggctttatctccaagcccttgccaatgggtgcaattgcaatttttgcattggcggttactgcactaactggaacactatcaattgaggatacattaagcggattcgggaataagaccatttggcttatcgttatcgcattctttatttcccggggatttatcaaaaccggtctcggtgcgagaatttcgtatgtattcgttcagaaattcggaaaaaaaacccttggactttcttattcactgctattcagtgatttaatactttcacctgctattccaagtaatacggcgcgtgcaggaggcattatatttcctattatcagatcattatccgaaacattcggatcaagcccggcaaatggaacagagagaaaaatcggtgcattcttattaaaaaccggttttcaggggaatctgatcacatctgctatgttcctgacagcgatggcggcgaacccgctgattgccaagctggcccatgatgtcgcaggggtggacttaacatggacaagctgggcaattgccgcgattgtaccgggacttgtaagcttaatcatcacgccgcttgtgatttacaaactgtatccgccggaaatcaaagaaacaccggatgcggcgaaaatcgcaacagaaaaactgaaagaaatgggaccgttcaaaaaatcggagctttccatggttatcgtgtttcttttggtgcttgtgctgtggatttttggcggcagcttcaacatcgacgctaccacaaccgcattgatcggtttggccgttctcttattatcacaagttctgacttgggatgatatcaagaaagaacagggcgcttgggatacgctcacttggtttgcggcgcttgtcatgctcgccaacttcttgaatgaattaggcatggtgtcttggttcagtaatgccatgaaatcatccgtatcagggttctcttggattgtggcattcatcattttaattgttgtgtattattactctcactatttctttgcaagtgcgacagcccacatcagtgcgatgtattcagcatttttggctgtcgtcgtggcagcgggcgcaccgccgcttttagcagcgctgagcctcgcgttcatcagcaacctgttcgggtcaacgactcactacggttctggagcggctccggtcttcttcggagcaggctacatcccgcaaggcaaatggtggtccatcggatttatcctgtcgattgttcatatcatcgtatggcttgtgatcggcggattatggtggaaagtactaggaatatggtagaaagaaaaaggcagacgcggtctgcctttttttattttcactccttcgtaagaaaatggattttgaaaaatgagaaaattccctgtgaaaaatggtatgatctaggtagaaaggacggctggtgctgtggtgaaaaagcggttccatttttccctgcaaacaaaaataatggggctgattgcggctctgctggtctttgtcattggtgtgctgaccattacgttagccgttcagcatacacagggagaacggagacaggcagagcagctggcggttcaaacggcgagaaccatttcctatatgccgccggttaaagagctcattgagagaaaagacggacatgcggctcagacgcaagaggtcattgaacaaatgaaagaacagactggtgcgtttgccatttatgttttgaacgaaaaaggagacattcgcagcgcctctggaaaaagcggattaaagaaactggagcgcagcagagaaattttgtttggcggttcgcatgtttctgaaacaaaagcggatggacgaagagtgatcagagggagcgcgccgattataaaagaacagaagggatacagccaagtgatcggcagcgtgtctgttgattttctgcaaacggagacagagcaaagcatcaaaaagcatttgagaaatttgagtgtgattgctgtgcttgtactgctgctcggatttattggcgccgccgtgctggcgaaaagcatcagaaaggatacgctcgggcttgaaccgcatgagatcgcggctctatatcgtgagaggaacgcaatgcttttcgcgattcgagaagggattattgccaccaatcgtgaaggcgtcgtcaccatgatgaacgtatcggcggccgagatgctgaagctgcccgagcctgtgatccatcttcctatagatgacgtcaaatgctgccgaaccaggaagtaagcgtcaacgatcaagtgtttattatcaatacgaaagtgatgaatcaaggcgggcaggcgtatgggattgtcgtcagcttcagggagaaaacagagctgaagaagctgatcgacacattgacagaggttcgcaaatattcagaggatctcagggcgcagactcatgaattttcaaataagctttatgcgattttagggctgcgtcga

SEQ ID NO: 2 - protein, 1050 aa, Paenibacillus sp-62047
IAGVVQSVNVSQAGYSSNDFKTATVTASDKLSDTSYQILQGTTVIATGTMKDEGYVWGKYVYSIDFSSVTATGTNFTIRSNGVSSYTFPIQTNMWNEYKDEMTAFYRLLRTTDTFAAYPAGYSNIAPSNKILHPDSFLDDAFSPDRTTHYDLTGGWFDAGDYGKYGGNQWVQGNIAISYLRHASSAAVNFDKDTNGIPDLVDEAIFGSQYLVKFANQLGGAIHNILRKGGFVLPHKVTDNVPGNTDDRALEAVEAVGGSGKSSGSLAATARAIRTAIAGGKVAANKVAQLQTLANEFQAAAIIFYNYTLTHQSGNHGSYGTMNNGGIANPLLWAEVQLYLLTGDAAYKTQAQTRINAINEAYVSSTNYWDMHPIALAEFYPVADSAIKTKIQSILKHQAYYFITLMDETPYGVLNQFGNFGVNEPHASYMADLLRYYELFNDPVALRAAKKALYWIVGNNPWNISWVSGVGSNFTDFLHTRLDEEAYSQTNTGVVLPGAMVSGPNIKDPNNKLSSSPWYEDKPIWADDTNQWRYNEYSVSIQTGLFYTIMGLSALGGNASTGGAEPVKLPITWPIIGDYVTGDVTVFAQPEGSLSNVSANGIVLSPSDGVYTTTVSTSADAPYTERKVQIKGTDDSGFTTYSNTHFTVAPALPDPSHPLLFDDFNQKGIWGSQKLDWVNWYNQNGGTASYTRTTVDTRTVGKFAHTPAATTSKAKFQPWKYNANLNGYRYLNFTMKNPGYPNTKIRIAANDGTKSVNLTSGEVAISSTWTTYQYDLNLHPTLNKSNVLIEVWLSNPTAGAYGEILIDEISAVNTNSGTAPTLSATGVNASIGNQSTVFTYTATYTDANNQAPFDVQVVIDGVIRSMTAADPTDTTYSDGRVYTYATTLPVGTHKFYFRTTDTTTNFVSTSVQTGPTVIRNKLEAEVLSINLTNYTHAVKDNADASGGKYRLFNGRQANDYIEYAVNVPKAGTYQVSARAMRLSDNGIYQLQINGSNQGTPFDTYQSSGKYLDYALGNVTITSPGTQLFRFKVTGKNASSLGYKLPLDFIQ

SEQ ID NO: 3 - DNA, 87 bp, Bacillus licheniformis
atgaaacaacaaaaacggctttacgcccgattgctgacgctgttatttgcgctcatcttcttgctgcctcattctgcagcagcagcg

SEQ ID NO: 4 - protein, 29 aa, Bacillus licheniformis
MKQQKRLYARLLTLLFALIFLLPHSAAAA

SEQ ID NO: 5 - DNA, 3111 bp, Paenibacillus sp.
gacgagtttgacacgctaagggaaaagtataaggccatgctgaacggagggacaacctataatctctccgacccggatatagcggcgcgtgttaatgccattacggtgactgcccagggatactgggactccatgcttaaagatccgaaccgtaaccgtctttggaacgatgcaccctttggctcggattcgacttccatcaccacgacctacagacacctttatgatatggcgctagcttatacgacttatggctccagtctgcagggcaatgccgcacttaaagcggatattatcagcggtttggactggatgaatgccaatcaattttataatggctgcagccaatatcaaaactggtggcactggcaaattggcggtcccatggccttgaatgatatcgtggcattaatgtacacggagctaaccgcaacacaaatttccaattacatggcggccatttattacacccaagcgagtgttacgatgacgggggcaaaccggctatgggaaagtcaggttattgccatctccggaatcttgaataaggattccgccagagttgccgctggtcgggatggcatcagcgctttgctgccgtatgtcgccaagggtgacggattttacaacgatggatcattcgttcagcatacttattatgcttacaacggtggttatggttcagagctgttatctggcattgcagacttgatatttattttgaatggctcttcatggcaggtaacggatcctaataaaaacaatgtataccgttggatttatgattcctacgagcctttcatctataaagggaatctgatggacatggtccgcggtagagagatctcaaggcatggattgcaggacgataaggcagccgtgactgtgatggcatcgatcattcgtctgtcacaaaccgctgcttccgccgatgctaccgcatttaagagaatggtgaaatattggctgctgctggatacggataagactttccttaaagcagtatcgattgatctgattattgccgcgaaccaactggtgaacgattccaccgttacctctcgaggggagctagtgaaatataaacaattctccggaatggaccgcgctgtacagcttagacctggcttcggttttgggcttagcatgttttccagccggatcggtaattatgagtcgattaatgcagagaacaacaaaggctggcataccggcgacggcatgacctacctttacaatactgacctgagtcagttcaatgaccatttctgggcaactgtggataattaccgattgccgggtaccacagtgctccagaacacgacgcaaaccgcgaacagccgcagcgacaaaagctgggccggaggaacggatattcttgggcaatatggtgtttccggcatggaactgcataccgtaggtaagagcctgacagccaagaaatcctggttcatgtttgacgatgagatcgtcgcgctgggttcaggtattgccagcaccgatggcatcgcaaccgaaacgattgtagagaatcgaaagctcaatagcagcggcaataatgcattgattgttaacgggacggcgaagccgggctcccttggatggtcggaaacaatgaccggaaccaattatattcatctagccggcagcgtacccggctccgatatcggttattattttcctggtggagcagcagtcaaaggcttgcgtgaagcccggtcgggaagctggagctcgctgaattcctccgcatcctggaaggactcgacattgcatacacgcaactttatgacgctttggttcgatcatggcatgaacccgacaaacggtagttattcttatgtgctgcttccgaataagaccagcagtgcggtggccagctatgctgcaacgcctcagatcagcattctggagaattctagctcggcgcaagcggtgaaggagacgcaattgaatgtcaccggaattaacttttggaacgatgagccaaccacggtgggcctggttacttccaatcggaaagcatccgttatgacaaaagaaacggctagtgatttcgagatatccgtttccgacccgacccaaagtaatgtggggaccatctatattgatgtcaacaaaagtgcaaccggattgatttcgaaggataatgaaataacggtcattcagtactacccaaccatgaagtttaaagtcaatgtaaacaattctggcgggaagtcctataaagtaaagtttagcctgacaggaacacccggcagcaacccgtctccaatcccgataccgaatccttacgaagcggaagctttgccaattaacgctctgacagatactcccgtggtttacaatgatgccaatgccagtggtggcaagaagcttggcttcaataacaatgcagtggatgattatgtggagttcagtctggacgtcacacagcccggcacctacgatgtcaaatcccggattatgaaatcaacgaacagcgggatttatcagctgtctattaatgggaccaacgtagggagcgcgcaggatatgttctggacgacctccgagctgtctaaggagtttactatgggctcatacagcttcagcacacccgggagctatttgttccgattaaaaacaaccggcaagaatgtcagttcttcaggatataagctgatgctggacaattttagtctggtatcaacaggtattgatacaacggtgattgtggacaatgccgatgcagctggtgttacgaaggtgggtacttggaccggaaccaatacgcagaccgatcggtacggcgccgactacattcacgatgggaacacggggaaaggtacgaagagcgttacctttactccaaatgtacctatcagtggaacttatcaggtttacatgatgtgggctgcccatacgaatagggcaacgaatgttcccgtagacgtaacgcattcaggcggtacagcaacgctaaatgttaaccaacaaggtaatggtggtgtgtggaatttactgggtacgtatagctttaatgctgggtccacgggggctatcaagatccgtacggacgcgacgaatggatatgttgtagccgatgccgtgaagctggtaaaggtccca

SEQ ID NO: 6 - protein, 1037 aa, Paenibacillus sp.
DEFDTLREKYKAMLNGGTTYNLSDPDIAARVNAITVTAQGYWDSMLKDPNRNRLWNDAPFGSDSTSITTTYRHLYDMALAYTTYGSSLQGNAALKADIISGLDWMNANQFYNGCSQYQNWWHWQIGGPMALNDIVALMYTELTATQISNYMAAIYYTQASVTMTGANRLWESQVIAISGILNKDSARVAAGRDGISALLPYVAKGDGFYNDGSFVQHTYYAYNGGYGSELLSGIADLIFILNGSSWQVTDPNKNNVYRWIYDSYEPFIYKGNLMDMVRGREISRHGLQDDKAAVTVMASIIRLSQTAASADATAFKRMVKYWLLLDTDKTFLKAVSIDLIIAANQLVNDSTVTSRGELVKYKQFSGMDRAVQLRPGFGFGLSMFSSRIGNYESINAENNKGWHTGDGMTYLYNTDLSQFNDHFWATVDNYRLPGTTVLQNTTQTANSRSDKSWAGGTDILGQYGVSGMELHTVGKSLTAKKSWFMFDDEIVALGSGIASTDGIATETIVENRKLNSSGNNALIVNGTAKPGSLGWSETMTGTNYIHLAGSVPGSDIGYYFPGGAAVKGLREARSGSWSSLNSSASWKDSTLHTRNFMTLWFDHGMNPTNGSYSYVLLPNKTSSAVASYAATPQISILENSSSAQAVKETQLNVTGINFWNDEPTTVGLVTSNRKASVMTKETASDFEISVSDPTQSNVGTIYIDVNKSATGLISKDNEITVIQYYPTMKFKVNVNNSGGKSYKVKFSLTGTPGSNPSPIPIPNPYEAEALPINALTDTPVVYNDANASGGKKLGFNNNAVDDYVEFSLDVTQPGTYDVKSRIMKSTNSGIYQLSINGTNVGSAQDMFWTTSELSKEFTMGSYSFSTPGSYLFRLKTTGKNVSSSGYKLMLDNFSLVSTGIDTTVIVDNADAAGVTKVGTWTGTNTQTDRYGADYIHDGNTGKGTKSVTFTPNVPISGTYQVYMMWAAHTNRATNVPVDVTHSGGTATLNVNQQGNGGVWNLLGTYSFNAGSTGAIKIRTDATNGYVVADAVKLVKVP